(19) United States
(12) Patent Application Publication
(10) Pub. No.: US 2025/0276016 A1
(43) Pub. Date: Sep. 4, 2025

(54) CHIMERIC ANTIGEN RECEPTOR AND IL-18 RECEPTOR COMPOSITIONS AND METHODS OF USE THEREIN

(71) Applicant: Minerva Biotechnologies Corporation, Waltham, MA (US)

(72) Inventors: Cynthia Bamdad, Boston, MA (US); Benoit Smagghe, Honolulu, HI (US)

(21) Appl. No.: 18/863,779

(22) PCT Filed: May 8, 2023
(86) PCT No.: PCT/US2023/066727

§ 371 Date: Nov. 7, 2024

Related U.S. Application Data
(60) Provisional application No. 63/339,961, filed on May 9, 2022.

(51) Int. Cl. A61K 35/17; A61K 40/11; A61K 40/31; A61K 40/42; C07K 14/705; C07K 14/715; C07K 14/725; C07K 16/30; C12N 5/0783

(52) U.S. Cl. CPC A61K 35/17; A61K 40/11; A61K 40/31; A61K 40/4257; C07K 14/7051; C07K 14/70521; C07K 14/7155; C07K 16/3092; C12N 5/0636; A61K 2239/13; A61K 2239/17; A61K 2239/21; C07K 2317/24; C07K 2317/53; C07K 2317/565; C07K 2317/622; C07K 2317/73; C07K 2319/02; C07K 2319/03

(57) ABSTRACT

Disclosed herein are compositions comprising an interleukin-18 (IL-18) receptor amino acid sequence that comprises an IL-18 receptor extracellular domain or a polynucleotide that encodes an IL-18 receptor amino acid sequence that comprises an IL-18 receptor extracellular domain and a chimeric antigen receptor (CAR) amino acid sequence or a polynucleotide that encodes a CAR amino acid sequence. In some embodiments the CAR targets MUC1 or MUC1*.

### CROSS REFERENCE

The present application claims the benefit of U.S. Provisional Application No. 63/339,961, filed on May 9, 2022, which is incorporated by reference in its entirety.

### SEQUENCE LISTING

The instant application contains a Sequence Listing which has been submitted electronically in XML format and is hereby incorporated by reference in its entirety. Said XML copy, created on May 8, 2023, is named 56699-761.601_SL.xml and is 585,728 bytes in size.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY

Disclosed herein are immune cells comprising (a) a first polynucleotide that encodes an interleukin-18 (IL-18) receptor amino acid sequence that comprises an IL-18 receptor extracellular domain; and (b) a second polynucleotide that encodes a chimeric antigen receptor (CAR) amino acid sequence. In some embodiments, the IL-18 receptor amino acid sequence comprises an IL-18 receptor alpha amino acid sequence. In some embodiments, the IL-18 receptor extracellular domain comprises an amino acid sequence with at least 90% sequence identity to SEQ ID NO: 2. In some embodiments, the IL-18 receptor extracellular domain comprises the amino acid sequence of SEQ ID NO: 2. In some embodiments, the IL-18 receptor alpha amino acid sequence comprises a transmembrane IL-18 receptor alpha amino acid sequence. In some embodiments, the transmembrane IL-18 receptor alpha amino acid sequence comprises an amino acid sequence with at least 90% sequence identity to SEQ ID NO: 3. In some embodiments, the transmembrane IL-18 receptor alpha amino acid sequence comprises the amino acid sequence of SEQ ID NO: 3. In some embodiments, the IL-18 receptor alpha amino acid sequence comprises an intracellular IL-18 receptor alpha amino acid sequence. In some embodiments, the intracellular IL-18 receptor alpha amino acid sequence comprises an amino acid sequence with at least 90% sequence identity to SEQ ID NO: 4. In some embodiments, the intracellular IL-18 receptor alpha amino acid sequence comprises the amino acid sequence of SEQ ID NO: 4. In some embodiments, the IL-18 receptor alpha amino acid sequence comprises an amino acid sequence with at least 90% sequence identity to SEQ ID NO: 5. In some embodiments, the IL-18 receptor alpha amino acid sequence comprises the amino acid sequence of SEQ ID NO: 5. In some embodiments, the first polynucleotide comprises the polynucleotide sequence with at least 90% identity to SEQ ID NO: 6. In some embodiments, the first polynucleotide comprises the polynucleotide sequence according to SEQ ID NO: 6. In some embodiments, the IL-18 receptor amino acid sequence comprises an IL-18 receptor beta amino acid sequence. In some embodiments, the IL-18 receptor extracellular domain comprises an amino acid sequence with at least 90% sequence identity to SEQ ID NO: 8. In some embodiments, the IL-18 receptor extracellular domain comprises the amino acid sequence of SEQ ID NO: 8. In some embodiments, the IL-18 receptor beta amino acid sequence comprises a transmembrane IL-18 receptor beta amino acid sequence. In some embodiments, the transmembrane IL-18 receptor beta amino acid sequence comprises an amino acid sequence with at least 90% sequence identity to SEQ ID NO: 9. In some embodiments, the transmembrane IL-18 receptor beta amino acid sequence comprises the amino acid sequence of SEQ ID NO: 9. In some embodiments, the IL-18 receptor beta amino acid sequence comprises an intracellular IL-18 receptor beta amino acid sequence. In some embodiments, the intracellular IL-18 receptor beta amino acid sequence comprises an amino acid sequence with at least 90% sequence identity to SEQ ID NO: 10. In some embodiments, the intracellular IL-18 receptor beta amino acid sequence comprises the amino acid sequence of SEQ ID NO: 10. In some embodiments, the IL-18 receptor beta amino acid sequence comprises an amino acid sequence with at least 90% sequence identity to SEQ ID NO: 11. In some embodiments, the IL-18 receptor beta amino acid sequence comprises the amino acid sequence of SEQ ID NO: 11. In some embodiments, the first polynucleotide comprises the polynucleotide sequence with at least 90% identity to SEQ ID NO: 12. In some embodiments, the first polynucleotide comprises the polynucleotide sequence according to SEQ ID NO: 12. In some embodiments, expression of the first polynucleotide is inducible. In some embodiments, expression of the first polynucleotide is inducible when the CAR is stimulated by an antigen. In some embodiments, expression of the first polynucleotide is operably linked to an inducible system. In some embodiments, the inducible system comprises a nuclear factor of activated T-cells (NFAT) response element. In some embodiments, the inducible system comprises 1 NFAT response element, 2 NFAT response elements, 3 NFAT response elements, 4 NFAT response elements, 5 NFAT response elements, or 6 NFAT response elements. In some embodiments, the NFAT response element comprises a FOXP3 NFAT response element. In some embodiments, the FOXP3 NFAT response element comprises the nucleotide sequence of SEQ ID NO: 13. In some embodiments, the inducible system comprises a repeat of 3 FOXP3 NFAT response elements. In some embodiments, the repeat of 3 FOXP3 NFAT response elements comprises the nucleotide sequence of SEQ ID NO: 14. In some embodiments, the inducible system comprises a repeat of 6 FOXP3 NFAT response elements. In some embodiments, the repeat of 6 FOXP3 NFAT response elements comprises the nucleotide sequence of SEQ ID NO: 15. In some embodiments, the inducible system comprises a minimum CMV promoter (mCMV). In some embodiments, the minimum CMV promoter (mCMV) comprises the nucleotide sequence of SEQ ID NO: 16. In some embodiments, the inducible system comprises the nucleotide sequence of SEQ ID NO: 17 or SEQ ID NO: 18. In some embodiments, the NFAT response element comprises a IL2 NFAT response element. In some embodiments, the IL2 NFAT response element comprises the nucleotide sequence of SEQ ID NO: 19. In some embodiments, the inducible system comprises a repeat of 3 IL2 NFAT response elements. In some embodiments, the repeat of 3 IL2 NFAT response elements comprises the nucleotide sequence of SEQ ID NO: 20. In some embodiments, the inducible system comprises a repeat of 6 IL2 NFAT response elements. In some embodiments, the repeat of 6 IL2 NFAT response elements comprises the nucleotide sequence of SEQ ID NO: 21. In some embodiments, the inducible system comprises the nucleotide sequence of SEQ ID NO: 22 or SEQ ID NO: 23. In some embodiments, the immune cell further comprises a third polynucleotide that encodes a second IL-18 receptor amino acid sequence that comprises an IL-18 receptor extracellular domain. In some embodiments, the second IL-18 receptor amino acid sequence comprises an IL-18 receptor alpha amino acid sequence. In some embodiments, the second IL-18 receptor amino acid sequence comprises an amino acid sequence with at least 90% sequence identity to any one of SEQ ID NOs: 1-6. In some embodiments, the second IL-18 receptor amino acid sequence comprises the amino acid sequence of any one of SEQ ID NOs: 1-6. In some embodiments, the third polynucleotide comprises the polynucleotide sequence with at least 90% identity to any one of SEQ ID NO: 6. In some embodiments, the third polynucleotide comprises the polynucleotide sequence according to any one of SEQ ID NO: 6. In some embodiments, the second IL-18 receptor amino acid sequence comprises an IL-18 receptor beta amino acid sequence. In some embodiments, the second IL-18 receptor amino acid sequence comprises an amino acid sequence with at least 90% sequence identity to any one of SEQ ID NOs: 7-11. In some embodiments, the second IL-18 receptor amino acid sequence comprises the amino acid sequence of any one of SEQ ID NOs: 7-11. In some embodiments, the third polynucleotide comprises the polynucleotide sequence with at least 90% identity to any one of SEQ ID NO: 12. In some embodiments, the third polynucleotide comprises the polynucleotide sequence according to any one of SEQ ID NO: 12. In some embodiments, the first polynucleotide comprises the polynucleotide sequence with at least 90% identity to SEQ ID NO: 26. In some embodiments, the first polynucleotide comprises the polynucleotide sequence according to SEQ ID NO: 26. In some embodiments, the first polynucleotide comprises the polynucleotide sequence with at least 90% identity to SEQ ID NO: 27. In some embodiments, the first polynucleotide comprises the polynucleotide sequence according to SEQ ID NO: 27. In some embodiments, the first polynucleotide comprises the polynucleotide sequence with at least 90% identity to SEQ ID NO: 28. In some embodiments, the first polynucleotide comprises the polynucleotide sequence according to SEQ ID NO: 28. In some embodiments, the first polynucleotide comprises the polynucleotide sequence with at least 90% identity to SEQ ID NO: 29. In some embodiments, the first polynucleotide comprises the polynucleotide sequence according to SEQ ID NO: 29. In some embodiments, the first polynucleotide and the third polynucleotide are contained within a polynucleotide sequence with at least 90% identity to SEQ ID NO: 30. In some embodiments, the first polynucleotide and the third polynucleotide are contained within the polynucleotide sequence according to SEQ ID NO: 30. In some embodiments, the first polynucleotide and the third polynucleotide are contained within a polynucleotide sequence with at least 90% identity to SEQ ID NO: 31. In some embodiments, the first polynucleotide and the third polynucleotide are contained within the polynucleotide sequence according to SEQ ID NO: 31. In some embodiments, the first polynucleotide and the third polynucleotide are contained within a polynucleotide sequence with at least 90% identity to SEQ ID NO: 32. In some embodiments, the first polynucleotide and the third polynucleotide are contained within the polynucleotide sequence according to SEQ ID NO: 32. In some embodiments, the first polynucleotide and the third polynucleotide are contained within a polynucleotide sequence with at least 90% identity to SEQ ID NO: 33. In some embodiments, the first polynucleotide and the third polynucleotide are contained within the polynucleotide sequence according to SEQ ID NO: 33. In some embodiments, the CAR amino acid sequence comprises a targeting moiety. In some embodiments, the targeting moiety binds to MUC1*. In some embodiments, the targeting moiety binds to the amino acid sequence according to SEQ ID NO: 35. In some embodiments, the targeting moiety bind to the amino acid sequence according to SEQ ID NO: 36. In some embodiments, the targeting moiety comprises a heavy chain variable domain comprising heavy chain complementarity determining regions (CDR)s HC-CDR1, HC-CDR2, and HC-CDR3 and the targeting moiety comprises a light chain variable domain comprising light chain CDRs LC-CDR1, LC-CDR2, and LC-CDR3. In some embodiments, the HC-CDR1, HC-CDR2, and HC-CDR3 and the LC-CDR1, LC-CDR2, and LC-CDR3 comprise the amino acid sequences according to: HC-CDR1: SEQ ID NO: 37; HC-CDR2: SEQ ID NO: 38; HC-CDR3: SEQ ID NO: 39; LC-CDR1: SEQ ID NO: 40; LC-CDR2: SEQ ID NO: 41; and LC-CDR3: SEQ ID NO:42. In some embodiments, the heavy chain variable domain comprises an amino acid sequence with at least 90% sequence identity to SEQ ID NO: 43 and the light chain variable domain comprises an amino acid sequence with at least 90% sequence identity to SEQ ID NO: 44. In some embodiments, the heavy chain variable domain comprises the amino acid sequence of SEQ ID NO: 43 and the light chain variable domain comprises the amino acid sequence of SEQ ID NO: 44. In some embodiments, the targeting moiety comprises a single chain variable fragment (scFv). In some embodiments, the scFv comprises an amino acid sequence with at least 90% sequence identity to SEQ ID NO: 45. In some embodiments, the scFv comprises the amino acid sequence of SEQ ID NO: 45. In some embodiments, the HC-CDR1, HC-CDR2, and HC-CDR3 and the LC-CDR1, LC-CDR2, and LC-CDR3 comprise the amino acid sequences according to: HC-CDR1: SEQ ID NO: 46; HC-CDR2: SEQ ID NO: 47; HC-CDR3: SEQ ID NO: 48; LC-CDR1: SEQ ID NO: 49; LC-CDR2: SEQ ID NO: 50; and LC-CDR3: SEQ ID NO: 51. In some embodiments, the heavy chain variable domain comprises an amino acid sequence with at least 90% sequence identity to SEQ ID NO: 52 and the light chain variable domain comprises an amino acid sequence with at least 90% sequence identity to SEQ ID NO: 53. In some embodiments, the heavy chain variable domain comprises the amino acid sequence of SEQ ID NO: 52 and the light chain variable domain comprises the amino acid sequence of SEQ ID NO: 53. In some embodiments, the targeting moiety comprises a single chain variable fragment (scFv). In some embodiments, the scFv comprises an amino acid sequence with at least 90% sequence identity to SEQ ID NO: 54. In some embodiments, the scFv comprises the amino acid sequence of SEQ ID NO: 54. In some embodiments, the HC-CDR1, HC-CDR2, and HC-CDR3 and the LC-CDR1, LC-CDR2, and LC-CDR3 comprise the amino acid sequences according to: HC-CDR1: SEQ ID NO: 55; HC-CDR2: SEQ ID NO: 56; HC-CDR3: SEQ ID NO: 57; LC-CDR1: SEQ ID NO: 58; LC-CDR2: SEQ ID NO: 59; and LC-CDR3: SEQ ID NO:60. In some embodiments, the heavy chain variable domain comprises an amino acid sequence with at least 90% sequence identity to SEQ ID NO: 61 and the light chain variable domain comprises an amino acid sequence with at least 90% sequence identity to SEQ ID NO: 62. In some embodiments, the heavy chain variable domain comprises the amino acid sequence of SEQ ID NO: 61 and the light chain variable domain comprises the amino acid sequence of SEQ ID NO: 62. In some embodiments, the targeting moiety comprises a single chain variable fragment (scFv). In some embodiments, the scFv comprises an amino acid sequence with at least 90% sequence identity to SEQ ID NO: 63. In some embodiments, the scFv comprises the amino acid sequence of SEQ ID NO: 63. In some embodiments, the CAR amino acid sequence comprises a hinge amino acid sequence. In some embodiments, the hinge amino acid sequence comprises a CD8 amino acid sequence. In some embodiments, the hinge amino acid sequence comprises an amino acid sequence with at least 90% sequence identity to SEQ ID NO: 64. In some embodiments, the hinge amino acid sequence comprises the amino acid sequence of SEQ ID NO: 64. In some embodiments, the hinge amino acid sequence comprises a CD28 amino acid sequence. In some embodiments, the hinge amino acid sequence comprises an amino acid sequence with at least 90% sequence identity to SEQ ID NO: 65. In some embodiments, the hinge amino acid sequence comprises the amino acid sequence of SEQ ID NO: 65. In some embodiments, the CAR amino acid sequence comprises a transmembrane amino acid sequence. In some embodiments, the transmembrane amino acid sequence comprises a CD8 amino acid sequence. In some embodiments, the transmembrane amino acid sequence comprises an amino acid sequence with at least 90% sequence identity to SEQ ID NO: 66. In some embodiments, the transmembrane amino acid sequence comprises the amino acid sequence of SEQ ID NO: 66. In some embodiments, the transmembrane amino acid sequence comprises a CD28 amino acid sequence. In some embodiments, the transmembrane amino acid sequence comprises an amino acid sequence with at least 90% sequence identity to SEQ ID NO: 67. In some embodiments, the transmembrane amino acid sequence comprises the amino acid sequence of SEQ ID NO: 67. In some embodiments, the CAR amino acid sequence comprises a co-stimulatory amino acid sequence. In some embodiments, the co-stimulatory amino acid sequence comprises a 41-BB amino acid sequence. In some embodiments, the co-stimulatory amino acid sequence comprises an amino acid sequence with at least 90% sequence identity to SEQ ID NO: 68. In some embodiments, the co-stimulatory amino acid sequence comprises the amino acid sequence of SEQ ID NO: 68. In some embodiments, the co-stimulatory amino acid sequence comprises a CD28 amino acid sequence. In some embodiments, the co-stimulatory amino acid sequence comprises an amino acid sequence with at least 90% sequence identity to SEQ ID NO: 69. In some embodiments, the co-stimulatory amino acid sequence comprises the amino acid sequence of SEQ ID NO: 69. In some embodiments, the CAR amino acid sequence comprises a signaling amino acid sequence. In some embodiments, the signaling amino acid sequence comprises a CD3 amino acid sequence. In some embodiments, the signaling amino acid sequence comprises an amino acid sequence with at least 90% sequence identity to SEQ ID NO: 70. In some embodiments, the signaling amino acid sequence comprises the amino acid sequence of SEQ ID NO: 70. In some embodiments, the signaling amino acid sequence comprises a cytoplasmic region comprising a CD3ζ intracellular signaling domain comprising a mutation in an immunoreceptor tyrosine-based activation motif (ITAM). In some embodiments, the mutation in the ITAM comprises a point mutation at a tyrosine residue. In some embodiments, the tyrosine residue is mutated to a phenylalanine residue. In some embodiments, the mutation in the ITAM comprises a mutation to more than one ITAM of the CD3ζ intracellular signaling domain. In some embodiments, the mutation to more than one ITAM of the CD3, intracellular signaling domain comprises mutations to two ITAMs. In some embodiments, the two ITAMs comprises the second and third ITAM of the CD3ζ intracellular signaling domain. In some embodiments, the mutation in the ITAM comprises a single point mutation in at least two ITAMs of the CD3ζ intracellular signaling domain. In some embodiments, the mutation in the ITAM comprises two point mutations at two tyrosine residues wherein the two tyrosine residues are mutated to phenylalanine residues. In some embodiments, the signaling amino acid sequence comprises an amino acid sequence with at least 90% sequence identity to SEQ ID NO: 71. In some embodiments, the signaling amino acid sequence comprises the amino acid sequence of SEQ ID NO: 71. In some embodiments, the CAR amino acid sequence comprises a hinge amino acid sequence according to SEQ ID NO: 64, a transmembrane amino acid sequence according to SEQ ID NO: 66, a co-stimulatory amino acid sequence according to SEQ ID NO: 68, and a signaling amino acid sequence according to SEQ ID NO: 70. In some embodiments, the CAR amino acid sequence comprises a hinge amino acid sequence according to SEQ ID NO: 65, a transmembrane amino acid sequence according to SEQ ID NO: 67, a co-stimulatory amino acid sequence according to SEQ ID NO: 69, and a signaling amino acid sequence according to SEQ ID NO: 70. In some embodiments, the CAR amino acid sequence comprises a hinge amino acid sequence according to SEQ ID NO: 64, a transmembrane amino acid sequence according to SEQ ID NO: 66, a co-stimulatory amino acid sequence according to SEQ ID NO: 68, and a signaling amino acid sequence according to SEQ ID NO: 71. In some embodiments, the CAR amino acid sequence comprises a hinge amino acid sequence according to SEQ ID NO: 65, a transmembrane amino acid sequence according to SEQ ID NO: 67, a co-stimulatory amino acid sequence according to SEQ ID NO: 69, and a signaling amino acid sequence according to SEQ ID NO:71. In some embodiments, the CAR amino acid sequence comprises an amino acid sequence with at least 90% sequence identity to any one of SEQ ID NO: 72-83. In some embodiments, the CAR amino acid sequence comprises the amino acid sequence of any one of SEQ ID NO: 72-83. In some embodiments, the second polynucleotide comprises the polynucleotide sequence with at least 90% identity to any one of SEQ ID NO: 84-95. In some embodiments, the second polynucleotide comprises the polynucleotide sequence according to any one of SEQ ID NO: 84-95. In some embodiments, the first polynucleotide and the second polynucleotide are on the same vector. In some embodiments, the first polynucleotide and the second polynucleotide are on the same vector and the vector comprises a polynucleotide sequence of at least 90% identity to any one of SEQ ID NOs: 96-190. In some embodiments, the first polynucleotide and the second polynucleotide are on the same vector and the vector comprises a polynucleotide sequence of any one of SEQ ID NOs: 96-190. In some embodiments, the first polynucleotide and the second polynucleotide are on separate vectors. In some embodiments, the immune cell is a T cell. In some embodiments, the immune cell is autologous. In some embodiments, the immune cell is allogeneic.

Disclosed herein are vectors comprising the first polynucleotide sequence of the above embodiments.

Disclosed herein are vectors comprising the second polynucleotide sequence of the above embodiments.

Disclosed herein are vectors comprising the first polynucleotide sequence and the second polynucleotide sequence of the above embodiments.

Disclosed herein are methods of treating cancer in a subject in need thereof comprises administering the immune cell of any of the above embodiments to the subject. In some embodiments, the cancer comprises a blood cancer. In some embodiments, the cancer comprises a solid tumor. In some embodiments, the cancer comprises breast cancer, lung cancer, prostate cancer, ovarian cancer, colorectal cancer, pancreatic cancer. In some embodiments, the cancer is a classified as a stage 1 cancer. In some embodiments, the cancer is a classified as a stage 2 cancer. In some embodiments, the cancer is a classified as a stage 3 cancer. In some embodiments, the cancer is characterized by a low antigen expressing cell. In some embodiments, the cancer is a classified as a stage 4 cancer. In some embodiments, the cancer is characterized by a high antigen expressing cell.

Disclosed herein are methods of increasing CAR T persistence in a subject in need thereof comprising administering the immune cell of any of the above embodiments.

Disclosed herein are methods of inhibiting cancer recurrence in a subject in need thereof comprising administering the immune cell of any of the above embodiments.

Disclosed herein are compositions comprising the second polynucleotide of any of the above embodiments, wherein the second polynucleotide further comprises a leader sequence. In some embodiments, the leader sequence increases uptake of the second polynucleotide into an immune cell. In some embodiments, the leader sequence increases uptake of the second polynucleotide into a nucleus of an immune cell. In some embodiments, the second polynucleotide encodes an amino acid sequence according to SEQ ID NO: 34. In some embodiments, the second polynucleotide is targeted to a T-cell receptor α constant (TRAC) locus. In some embodiments, the second polynucleotide is targeted to a T-cell receptor α constant (TRAC) locus using CRISPR gene editing or TALEN gene editing.

Disclosed herein are pharmaceutical compositions comprising (a) the immune cell of any one of the above embodiments, and (b) a pharmaceutically acceptable excipient.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates the increased expression of IL-18 receptor alpha by CAR constructs that include an inducible IL-18 cytokine or a CAR bearing 1XX mutations in CD3ζ signaling domain.

FIGS. 2A-2D illustrate differences in expression IL-18 receptor alpha by different CAR T cell constructs with repeated antigen stimulation.

FIGS. 3A-3C illustrate differences in the ability to kill cancer cells expressing low targeted antigen for different CAR constructs.

FIG. 4 illustrates IL-18 receptor alpha expressed by Expi-CHO cells transfected with a plasmid, before and after PMA/ionomycin activation.

FIG. 5 illustrates IL-18Rα and IL-18Rβ expressed by Expi-CHO cells transfected with a plasmid, before and after PMA/ionomycin activation.

FIGS. 6A-6P illustrate the amount of IL-18Rα expressed by different CAR constructs that have or do noe have an inducible IL-18Rα, both before and after PMA/ionomycin activation translocates NFATs to nucleus.

FIGS. 7A-7F illustrate the differences in expression of IL-18Rα, when expressed from 2 viral vectors or 1 viral vector, comprising 6 NFAT response elements or 3 NFAT response elements.

FIGS. 8A-8O compare the loss of killing potency after 6-day antigen stimulation for CAR constructs with or without an inducible IL-18Rα.

FIGS. 9A-9B show measured percent CAR-positivity and CAR mean fluorescent intensity for various CAR constructs, before and after 6-day antigen stimulation.

FIGS. 10A-10H show fluorescent photographs of T47D wild-type cancer cells, which express low levels of MUC1*, in co-culture with MNC2-41BB CAR T cells, with and without inducible IL-18Rα, where the CAR T cells had been antigen stimulated for 6 days before the co-culture with target cells.

FIGS. 11A-11R show fluorescent photographs of T47D cancer cells doped with 5% cells engineered to express more MUC1*, in co-culture with MNC2-41BB CAR T cells for 24, 48 and 72 hours, with and without inducible IL-18Rα, where the CAR T cells had been antigen stimulated for 6 days before the co-culture with target cells.

FIGS. 12A-12F show killing potency of MNC2-41BB-3z with or without inducible IL-18Rα, where the CAR T cells had been antigen stimulated for 6 days before the co-culture with target cells.

FIGS. 13A-13R show fluorescent photographs of T47D cancer cells doped with 5% cells engineered to express more MUC1*, in co-culture with MNC2-28-3z CAR T cells, with and without inducible IL-18Rα, where the CAR T cells had been antigen stimulated for 6 days before the co-culture with target cells.

FIGS. 14A-14F show killing potency of MNC2-28-3z, with or without inducible IL-18Rα, where the CAR T cells had been antigen stimulated for 6 days before the co-culture with target cells.

FIGS. 15A-15H show fluorescent photographs of T47D cancer cells doped with 5% cells engineered to express more MUC1*, in co-culture with MNC2-28-1XX CAR T cells, with and without inducible IL-18Rα, where the CAR T cells had been antigen stimulated for 6 days before the co-culture with target cells.

FIGS. 16A-16F show killing potency of MNC2-28-1XX, with or without inducible IL-18Rα, where the CAR T cells had been antigen stimulated for 6 days before the co-culture with target cells.

FIGS. 17A-17D show killing potency of various CAR constructs, with or without inducible IL-18Rα, where the CAR T cells had been antigen stimulated for 6 days before co-culture with control, non-target cells.

### DETAILED DESCRIPTION OF THE INVENTION

Chimeric antigen receptors (CARs) are molecules that include a targeting moiety (such as an antibody single chain variable fragment scFv) specific for a tumor antigen, a transmembrane domain, and at least one T cell signaling moiety. When expressed on the surface of an immune cell, such as a T cell, the CARs mediate binding to a target and activate the T cells, ultimately inducing the target cell lysis. CAR T therapy is a promising approach to treat hematological malignancies, including non-Hodgkin lymphoma, B-cell acute lymphoblastic leukemia, and multiple myeloma. However, challenges have plagued CAR T therapy related to T cell persistence (also known as CAR T exhaustion), and efficacy concerns related to killing low antigen expressing cells. CAR T cells administered for the treatment of solid tumor cancers become exhausted rather quickly and lose the ability to kill the targeted cancer cells. Cancer recurrence, after CAR T cell treatment of solid tumor cancers, occurs sooner and much more frequently than in blood cancers. Therefore, the ability to improve CAR T cell persistence and resist exhaustion would allow for broader applicability of CAR T cell therapies to solid tumor cancers. Immune cells expressing a CAR and a cytokine have been described. A potential problem with an immune cell expressing a CAR and a cytokine is that cytokines induce expression of other cytokines, which can lead to unforeseen results, including cytokine storm. A solution, as described herein, is an immune cell that expresses the CAR and a cytokine receptor.

One approach for attenuating CAR T exhaustion is to mutate tyrosine residues in the immunoreceptor tyrosine-based activation motif (ITAM) s of the intracellular CD3ζ signaling domain of the CAR. In particular, as disclosed herein, mutation of tyrosine to phenylalanine in any of the ITAMS, such as in the second and third ITAM (known as 1XX), have an improved ability to recognize and kill low antigen expressing cells as compared with wild type CD3ζ signaling domains that do not have the ITAM mutations. Additionally, T cells expressing CARs with one or more ITAM mutations also have another important advantage in that IL-18 receptor alpha is not downregulated after repeated activation. As shown herein, T cells expressing the same CAR, but without one or more mutations in the ITAMs, downregulate the IL-18 receptor alpha after repeated activation and, as a consequence, markers of CAR T cell exhaustion go up and their killing potency goes down. To resist exhaustion, as disclosed herein, are immune cell compositions that express a CAR and an IL-18 receptor. Such compositions have improved efficacy and resist exhaustion even after repeated antigen activation.

A target for solid tumors that CAR T therapy can be applied to is MUC1 and MUC1* which is a cleaved form of MUC1. When MUC1 is cleaved to MUC1*, cleavage and release of the bulk of the extracellular domain of MUC1 unmasks a binding site for activating ligands dimeric NME1, NME6, NME7, NME7AB, NME7-X1 or NME8. It is an ideal target for cancer drugs as it is aberrantly expressed on over 75% of all cancers and is likely overexpressed to an even greater percentage of metastatic solid tumor cancers.

CAR T cells for the treatment of blood cancers have been very successful. However, CAR T cells for the treatment of solid tumor cancers, which make up 93% of all cancers, has been more challenging. One reason is that CAR T cells for blood cancers target CD19 that is expressed on all B cells and most blood cancers are B cell malignancies. However, there is no B cell equivalent on solid tumor cancer cells. Therefore, creating a CAR T cell for solid tumors that doesn't kill the normal cells has been a challenge. MNC2 is an antibody that binds to MUC1* that is selectively expressed on solid tumor cancer cells. Two additional technical challenges for CAR T treatments for solid tumor cancers are CAR T cell exhaustion and their inability to kill the cancer cells that express low levels of the target antigen. Cancer cells expressing low levels of target antigen are characteristic of early cancer cells, which are the cells that escape existing cancer treatments and cause cancer recurrence.

In animal models, immune cells that co-express a CAR and a cytokine have been shown to increase CAR T cell efficacy. However, expression of a cytokine can trigger induction of other cytokines, leading to cytokine storm. T cells expressing CAR constructs with one or more Tyrosines mutated to Phenylalanine in one or more of the three ITAMs that make up CD3° C. signaling tail were shown to increase CAR T cell persistence and inhibit CAR T cell exhaustion. We showed that T cells expressing a CAR with the 1XX mutations increased the ability of the CAR T cells to kill cells expressing low levels of the target antigen. We also showed that immune cells expressing both a CAR and an inducible IL-18 cytokine increased the ability of the CAR T cells to kill cancer cells expressing low levels of the target antigen. Our results, as disclosed herein, revealed that both immune cells expressing a CAR with 1XX mutations and immune cells expressing both a CAR and an inducible IL-18 cytokine expressed higher levels of IL-18Rα than the control immune cell bearing the same CAR without the 1XX mutations or the immune cell expressing a CAR but without the inducible IL-18 cytokine. Furthermore, their high level of IL-18Rα expression did not decline even after repeated antigen stimulation.

Here we demonstrate that immune cells that express both a CAR and an inducible IL-18Rα overcome CAR T cell exhaustion and also increase their ability to kill the cancer cells that express low levels of the target antigen.

Disclosed herein are immune cells comprising a) a first polynucleotide that encodes an interleukin-18 (IL-18) receptor amino acid sequence that comprises an IL-18 receptor extracellular domain; and b) a second polynucleotide that encodes a chimeric antigen receptor (CAR) amino acid sequence.

Disclosed herein are vectors comprising the first polynucleotide of the above embodiment.

Disclosed herein are vectors comprising the second polynucleotide sequence of the above embodiment.

Disclosed herein are vectors comprising the first polynucleotide sequence and the second polynucleotide sequence of the above embodiments.

Disclosed herein are methods of treating cancer in a subject in need thereof comprises administering the immune cell of any of the above embodiments.

Disclosed herein are methods of increasing CAR T persistence in a subject in need thereof comprising administering the immune cell of any of the above embodiments.

Disclosed herein are methods of inhibiting cancer recurrence in a subject in need thereof comprising administering the immune cell of any of the above embodiments.

First Polynucleotide that Encodes an IL-18 Receptor

### IL-18 Receptor

In some embodiments, the IL-18 receptor amino acid sequence comprises an IL-18 receptor alpha amino acid sequence. In some embodiments, the IL-18 receptor extracellular domain comprises an amino acid sequence with at least 85% sequence identity to SEQ ID NO: 2. In some embodiments, the IL-18 receptor extracellular domain comprises an amino acid sequence with at least 90% sequence identity to SEQ ID NO: 2. In some embodiments, the IL-18 receptor extracellular domain comprises an amino acid sequence with at least 95% sequence identity to SEQ ID NO: 2. In some embodiments, the IL-18 receptor extracellular domain comprises an amino acid sequence with at least 99% sequence identity to SEQ ID NO: 2. In some embodiments, the IL-18 receptor extracellular domain comprises the amino acid sequence of SEQ ID NO: 2. In some embodiments, the IL-18 receptor alpha amino acid sequence comprises a transmembrane IL-18 receptor alpha amino acid sequence. In some embodiments, the transmembrane IL-18 receptor alpha amino acid sequence comprises an amino acid sequence with at least 90% sequence identity to SEQ ID NO: 3. In some embodiments, the transmembrane IL-18 receptor alpha amino acid sequence comprises the amino acid sequence of SEQ ID NO: 3. In some embodiments, the IL-18 receptor alpha amino acid sequence comprises an intracellular IL-18 receptor alpha amino acid sequence. In some embodiments, the intracellular IL-18 receptor alpha amino acid sequence comprises an amino acid sequence with at least 85% sequence identity to SEQ ID NO: 4. In some embodiments, the intracellular IL-18 receptor alpha amino acid sequence comprises an amino acid sequence with at least 90% sequence identity to SEQ ID NO: 4. In some embodiments, the intracellular IL-18 receptor alpha amino acid sequence comprises an amino acid sequence with at least 95% sequence identity to SEQ ID NO: 4. In some embodiments, the intracellular IL-18 receptor alpha amino acid sequence comprises an amino acid sequence with at least 99% sequence identity to SEQ ID NO: 4. In some embodiments, the intracellular IL-18 receptor alpha amino acid sequence comprises the amino acid sequence of SEQ ID NO: 4. In some embodiments, the IL-18 receptor alpha amino acid sequence comprises an amino acid sequence with at least 85% sequence identity to SEQ ID NO: 5. In some embodiments, the IL-18 receptor alpha amino acid sequence comprises an amino acid sequence with at least 90% sequence identity to SEQ ID NO: 5. In some embodiments, the IL-18 receptor alpha amino acid sequence comprises an amino acid sequence with at least 95% sequence identity to SEQ ID NO: 5. In some embodiments, the IL-18 receptor alpha amino acid sequence comprises an amino acid sequence with at least 99% sequence identity to SEQ ID NO: 5. In some embodiments, the IL-18 receptor alpha amino acid sequence comprises the amino acid sequence of SEQ ID NO: 5. In some embodiments, the first polynucleotide comprises the polynucleotide sequence with at least 85% identity to SEQ ID NO: 6. In some embodiments, the first polynucleotide comprises the polynucleotide sequence with at least 90% identity to SEQ ID NO: 6. In some embodiments, the first polynucleotide comprises the polynucleotide sequence with at least 95% identity to SEQ ID NO: 6. In some embodiments, the first polynucleotide comprises the polynucleotide sequence with at least 99% identity to SEQ ID NO: 6. In some embodiments, the first polynucleotide comprises the polynucleotide sequence according to SEQ ID NO: 6.

In some embodiments, the IL-18 receptor amino acid sequence comprises an IL-18 receptor beta amino acid sequence. In some embodiments, the IL-18 receptor extracellular domain comprises an amino acid sequence with at least 85% sequence identity to SEQ ID NO: 8. In some embodiments, the IL-18 receptor extracellular domain comprises an amino acid sequence with at least 90% sequence identity to SEQ ID NO: 8. In some embodiments, the IL-18 receptor extracellular domain comprises an amino acid sequence with at least 95% sequence identity to SEQ ID NO: 8. In some embodiments, the IL-18 receptor extracellular domain comprises an amino acid sequence with at least 99% sequence identity to SEQ ID NO: 8. In some embodiments, the IL-18 receptor extracellular domain comprises the amino acid sequence of SEQ ID NO: 8. In some embodiments, the IL-18 receptor beta amino acid sequence comprises a transmembrane IL-18 receptor beta amino acid sequence. In some embodiments, the transmembrane IL-18 receptor beta amino acid sequence comprises an amino acid sequence with at least 90% sequence identity to SEQ ID NO: 9. In some embodiments, the transmembrane IL-18 receptor beta amino acid sequence comprises the amino acid sequence of SEQ ID NO: 9. In some embodiments, the IL-18 receptor beta amino acid sequence comprises an intracellular IL-18 receptor beta amino acid sequence. In some embodiments, the intracellular IL-18 receptor beta amino acid sequence comprises an amino acid sequence with at least 85% sequence identity to SEQ ID NO: 10. In some embodiments, the intracellular IL-18 receptor beta amino acid sequence comprises an amino acid sequence with at least 90% sequence identity to SEQ ID NO: 10. In some embodiments, the intracellular IL-18 receptor beta amino acid sequence comprises an amino acid sequence with at least 95% sequence identity to SEQ ID NO: 10. In some embodiments, the intracellular IL-18 receptor beta amino acid sequence comprises an amino acid sequence with at least 99% sequence identity to SEQ ID NO: 10. In some embodiments, the intracellular IL-18 receptor beta amino acid sequence comprises the amino acid sequence of SEQ ID NO: 10. In some embodiments, the IL-18 receptor beta amino acid sequence comprises an amino acid sequence with at least 85% sequence identity to SEQ ID NO: 11. In some embodiments, the IL-18 receptor beta amino acid sequence comprises an amino acid sequence with at least 90% sequence identity to SEQ ID NO: 11. In some embodiments, the IL-18 receptor beta amino acid sequence comprises an amino acid sequence with at least 95% sequence identity to SEQ ID NO: 11. In some embodiments, the IL-18 receptor beta amino acid sequence comprises an amino acid sequence with at least 99% sequence identity to SEQ ID NO: 11. In some embodiments, the IL-18 receptor beta amino acid sequence comprises the amino acid sequence of SEQ ID NO: 11. In some embodiments, the first polynucleotide comprises the polynucleotide sequence with at least 85% identity to SEQ ID NO: 12. In some embodiments, the first polynucleotide comprises the polynucleotide sequence with at least 90% identity to SEQ ID NO: 12. In some embodiments, the first polynucleotide comprises the polynucleotide sequence with at least 95% identity to SEQ ID NO: 12. In some embodiments, the first polynucleotide comprises the polynucleotide sequence with at least 99% identity to SEQ ID NO: 12. In some embodiments, the first polynucleotide comprises the polynucleotide sequence according to SEQ ID NO: 12.

TABLE 1
IL-18 Receptor Sequences

| | SEQ ID |
|---|---|
| Description | NO: |
| IL-18 receptor alpha signal peptide | 1 |
| IL-18 receptor alpha extracellular domain | 2 |
| transmembrane IL-18 receptor alpha | 3 |
| intracellular IL-18 receptor alpha | 4 |
| IL-18 receptor alpha (full length) | 5 |
| IL-18 receptor alpha (full length) polynucleotide | 6 |
| sequence | |
| IL-18 receptor beta signal peptide | 7 |
| IL-18 receptor beta extracellular domain | 8 |
| transmembrane IL-18 receptor beta | 9 |
| intracellular IL-18 receptor beta | 10 |
| IL-18 receptor beta (full length) | 11 |
| IL-18 receptor beta (full length) polynucleotide sequence | 12 |

### Inducible Expression Systems

In some embodiments, the expression of the first polynucleotide is inducible. In some embodiments, the expression of the first polynucleotide is inducible when the CAR is stimulated by an antigen. In some embodiments, the expression of the first polynucleotide is operably linked to an inducible system. In some embodiments, the inducible system comprises a nuclear factor of activated T-cells (NFAT) response element. The NFAT response element may be one to which the NFAT1, also known as NFATc2, NFAT2 also known as NFATc or NFATc1, NFAT3 also known as NFATc4, NFAT4 also known as NFATc3, or NFAT5 binds. In some embodiments, the NFAT that binds to the NFAT response element is NFATc1. In some embodiments, the inducible system comprises 1 NFAT response element, 2 NF AT response elements, 3 NF AT response elements, 4 NFAT response elements, 5 NFAT response elements, or 6 NFAT response elements. In some embodiments, the NFAT response element is located within a FOXP3 promoter region. In some embodiments, the NFAT response element that is located within a FOXP3 promoter region comprises the nucleotide sequence of SEQ ID NO: 13. In some embodiments, the inducible system comprises a repeat of 3 NFAT response elements that are located within a FOXP3 promoter region. In some embodiments, the repeat of 3 NFAT response elements that are located within a FOXP3 promoter region comprises a nucleotide sequence of at least 80% sequence identity to SEQ ID NO: 14. In some embodiments, the repeat of 3 NFAT response elements that are located within a FOXP3 promoter region comprises the nucleotide sequence of SEQ ID NO: 14. In some embodiments, the inducible system comprises a repeat of 6 NFAT response elements that are located within a FOXP3 promoter region. In some embodiments, the repeat of 6 NFAT response elements that are located within a FOXP3 promoter region comprises a nucleotide sequence with at least 85% sequence identity to SEQ ID NO: 15. In some embodiments, the repeat of 6 NFAT response elements that are located within a FOXP3 promoter region comprises a nucleotide sequence with at least 90% sequence identity to SEQ ID NO: 15. In some embodiments, the repeat of 6 NFAT response elements that are located within a FOXP3 promoter region comprises a nucleotide sequence with at least 95% sequence identity to SEQ ID NO: 15. In some embodiments, the repeat of 6 NFAT response elements that are located within a FOXP3 promoter region comprises a nucleotide sequence with at least 99% sequence identity to SEQ ID NO: 15. In some embodiments, the repeat of 6 NFAT response elements that are located within a FOXP3 promoter region comprises the nucleotide sequence of SEQ ID NO: 15. In some embodiments, the inducible system comprises a minimum CMV promoter (mCMV). In some embodiments, the minimum CMV promoter (mCMV) comprises the nucleotide sequence of SEQ ID NO: 16. In some embodiments, the inducible system comprises a nucleotide sequence with at least 85% identity to SEQ ID NO: 17 or a nucleotide sequence with at least 85% identity to SEQ ID NO: 18. In some embodiments, the inducible system comprises the nucleotide sequence of SEQ ID NO: 17 or SEQ ID NO: 18. In some embodiments, the inducible system comprises a nucleotide sequence with at least 90% identity to SEQ ID NO: 17 or a nucleotide sequence with at least 90% identity to SEQ ID NO: 18. In some embodiments, the inducible system comprises a nucleotide sequence with at least 95% identity to SEQ ID NO: 17 or a nucleotide sequence with at least 95% identity to SEQ ID NO: 18. In some embodiments, the inducible system comprises a nucleotide sequence with at least 99% identity to SEQ ID NO: 17 or a nucleotide sequence with at least 99% identity to SEQ ID NO: 18. In some embodiments, the NFAT response element is located within an IL2 promoter region. In some embodiments, the NFAT response element that is located within an IL2 promoter region comprises the nucleotide sequence of SEQ ID NO: 19. In some embodiments, the inducible system comprises a repeat of 3 NFAT response elements that are located within an IL2 promoter region. In some embodiments, the repeat of 3 NFAT response elements that are located within an IL2 promoter region comprises the nucleotide sequence of SEQ ID NO: 20. In some embodiments, the inducible system comprises a repeat of 6 NFAT response elements that are located within an IL2 promoter region. In some embodiments, the repeat of 6 NFAT response elements that are located within an IL2 promoter region comprises a nucleotide sequence with at least 85% sequence identity to SEQ ID NO: 21. In some embodiments, the repeat of 6 NFAT response elements that are located within an IL2 promoter region comprises a nucleotide sequence with at least 90% sequence identity to SEQ ID NO: 21. In some embodiments, the repeat of 6 NFAT response elements that are located within an IL2 promoter region comprises a nucleotide sequence with at least 95% sequence identity to SEQ ID NO: 21. In some embodiments, the repeat of 6 NF AT response elements that are located within an IL2 promoter region comprises a nucleotide sequence with at least 99% sequence identity to SEQ ID NO: 21. In some embodiments, the repeat of 6 NFAT response elements that are located within an IL2 promoter region comprises the nucleotide sequence of SEQ ID NO: 21. In some embodiments, the inducible system comprises a nucleotide sequence with at least 85% sequence identity to SEQ ID NO: 22 or with at least 85% sequence identity to SEQ ID NO: 23. In some embodiments, the inducible system comprises a nucleotide sequence with at least 90% sequence identity to SEQ ID NO: 22 or with at least 90% sequence identity to SEQ ID NO: 23. In some embodiments, the inducible system comprises a nucleotide sequence with at least 95% sequence identity to SEQ ID NO: 22 or with at least 95% sequence identity to SEQ ID NO: 23. In some embodiments, the inducible system comprises a nucleotide sequence with at least 99% sequence identity to SEQ ID NO: 22 or with at least 99% sequence identity to SEQ ID NO: 23. In some embodiments, the inducible system comprises the nucleotide sequence of SEQ ID NO: 22 or SEQ ID NO: 23.

TABLE 2
Inducible System Sequences

| Description | SEQ ID NO: |
|---|---|
| FOXP3 NFAT response element | 13 |
| 3XFOXP3NFAT | 14 |
| (repeat of3 FOXP3 NFAT response element) | |
| 6XFOXP3NFAT | 15 |
| (repeat of6 FOXP3 NFAT response element) | |
| Minimum CMV promoter (mCMV) | 16 |
| 3XFOXP3NFAT mCMV | 17 |
| 6XFOXP3NFAT | 18 |
| IL2 NFAT response element | 19 |
| 3XIL2NFAT | 20 |
| (repeat of3 IL2 NFAT response element) | |
| 6XIL2NFAT | 21 |
| (repeat of6 IL2 NFAT response element) | |
| 3XIL2NFAT mCMV | 22 |
| 6XIL2NFAT | 23 |
| T2A self-cleaving peptide, nucleotide sequence | 24 |
| T2A self-cleaving peptide, amino acid sequence | 25 |

### Third Polynucleotide

In some embodiments, the immune cell further comprises a third polynucleotide that encodes a second IL-18 receptor amino acid sequence that comprises an IL-18 receptor extracellular domain. In some embodiments, the second IL-18 receptor amino acid sequence comprises an IL-18 receptor alpha amino acid sequence. In some embodiments, the second IL-18 receptor amino acid sequence comprises an amino acid sequence with at least 85% sequence identity to any one of SEQ ID NOs: 1-5. In some embodiments, the second IL-18 receptor amino acid sequence comprises an amino acid sequence with at least 90% sequence identity to any one of SEQ ID NOs: 1-5. In some embodiments, the second IL-18 receptor amino acid sequence comprises an amino acid sequence with at least 95% sequence identity to any one of SEQ ID NOs: 1-5. In some embodiments, the second IL-18 receptor amino acid sequence comprises an amino acid sequence with at least 99% sequence identity to any one of SEQ ID NOs: 1-5. In some embodiments, the second IL-18 receptor amino acid sequence comprises the amino acid sequence of any one of SEQ ID NOs: 1-5. In some embodiments, the third polynucleotide comprises the polynucleotide sequence with at least 90% identity to any one of SEQ ID NO: 6. In some embodiments, the third polynucleotide comprises the polynucleotide sequence according to any one of SEQ ID NO: 6. In some embodiments, the second IL-18 receptor amino acid sequence comprises an IL-18 receptor beta amino acid sequence. In some embodiments, the second IL-18 receptor amino acid sequence comprises an amino acid sequence with at least 85% sequence identity to any one of SEQ ID NOs: 7-11. In some embodiments, the second IL-18 receptor amino acid sequence comprises an amino acid sequence with at least 90% sequence identity to any one of SEQ ID NOs: 7-11. In some embodiments, the second IL-18 receptor amino acid sequence comprises an amino acid sequence with at least 95% sequence identity to any one of SEQ ID NOs: 7-11. In some embodiments, the second IL-18 receptor amino acid sequence comprises an amino acid sequence with at least 99% sequence identity to any one of SEQ ID NOs: 7-11. In some embodiments, the second IL-18 receptor amino acid sequence comprises the amino acid sequence of any one of SEQ ID NOs: 7-11. In some embodiments, the third polynucleotide comprises the polynucleotide sequence with at least 85% identity to any one of SEQ ID NO: 12. In some embodiments, the third polynucleotide comprises the polynucleotide sequence with at least 90% identity to any one of SEQ ID NO: 12. In some embodiments, the third polynucleotide comprises the polynucleotide sequence with at least 95% identity to any one of SEQ ID NO: 12. In some embodiments, the third polynucleotide comprises the polynucleotide sequence with at least 99% identity to any one of SEQ ID NO: 12. In some embodiments, the third polynucleotide comprises the polynucleotide sequence according to any one of SEQ ID NO: 12.

### First Polynucleotide Constructs

In some embodiments, the first polynucleotide comprises the polynucleotide sequence with at least 90% identity to SEQ ID NO: 26. In some embodiments, the first polynucleotide comprises the polynucleotide sequence with at least 95% identity to SEQ ID NO: 26. In some embodiments, the first polynucleotide comprises the polynucleotide sequence with at least 99% identity to SEQ ID NO: 26. In some embodiments, the first polynucleotide comprises the polynucleotide sequence according to SEQ ID NO: 26. In some embodiments, the first polynucleotide comprises the polynucleotide sequence with at least 90% identity to SEQ ID NO: 27. In some embodiments, the first polynucleotide comprises the polynucleotide sequence with at least 95% identity to SEQ ID NO: 27. In some embodiments, the first polynucleotide comprises the polynucleotide sequence with at least 99% identity to SEQ ID NO: 27. In some embodiments, the first polynucleotide comprises the polynucleotide sequence according to SEQ ID NO: 27. In some embodiments, the first polynucleotide comprises the polynucleotide sequence with at least 90% identity to SEQ ID NO: 28. In some embodiments, the first polynucleotide comprises the polynucleotide sequence with at least 95% identity to SEQ ID NO: 28. In some embodiments, the first polynucleotide comprises the polynucleotide sequence with at least 99% identity to SEQ ID NO: 28. In some embodiments, the first polynucleotide comprises the polynucleotide sequence according to SEQ ID NO: 28. In some embodiments, the first polynucleotide comprises the polynucleotide sequence with at least 90% identity to SEQ ID NO: 29. In some embodiments, the first polynucleotide comprises the polynucleotide sequence with at least 95% identity to SEQ ID NO: 29. In some embodiments, the first polynucleotide comprises the polynucleotide sequence with at least 99% identity to SEQ ID NO: 29. In some embodiments, the first polynucleotide comprises the polynucleotide sequence according to SEQ ID NO: 29. In some embodiments, the first polynucleotide and the third polynucleotide are contained within a polynucleotide sequence with at least 90% identity to SEQ ID NO: 30. In some embodiments, the first polynucleotide and the third polynucleotide are contained within a polynucleotide sequence with at least 95% identity to SEQ ID NO: 30. In some embodiments, the first polynucleotide and the third polynucleotide are contained within a polynucleotide sequence with at least 99% identity to SEQ ID NO: 30. In some embodiments, the first polynucleotide and the third polynucleotide are contained within the polynucleotide sequence according to SEQ ID NO: 30. In some embodiments, the first polynucleotide and the third polynucleotide are contained within a polynucleotide sequence with at least 90% identity to SEQ ID NO: 31. In some embodiments, the first polynucleotide and the third polynucleotide are contained within a polynucleotide sequence with at least 95% identity to SEQ ID NO: 31. In some embodiments, the first polynucleotide and the third polynucleotide are contained within a polynucleotide sequence with at least 99% identity to SEQ ID NO: 31. In some embodiments, the first polynucleotide and the third polynucleotide are contained within the polynucleotide sequence according to SEQ ID NO: 31. In some embodiments, the first polynucleotide and the third polynucleotide are contained within a polynucleotide sequence with at least 90% identity to SEQ ID NO: 32. In some embodiments, the first polynucleotide and the third polynucleotide are contained within a polynucleotide sequence with at least 95% identity to SEQ ID NO: 32. In some embodiments, the first polynucleotide and the third polynucleotide are contained within a polynucleotide sequence with at least 99% identity to SEQ ID NO: 32. In some embodiments, the first polynucleotide and the third polynucleotide are contained within the polynucleotide sequence according to SEQ ID NO: 32. In some embodiments, the first polynucleotide and the third polynucleotide are contained within a polynucleotide sequence with at least 90% identity to SEQ ID NO: 33. In some embodiments, the first polynucleotide and the third polynucleotide are contained within a polynucleotide sequence with at least 95% identity to SEQ ID NO: 33. In some embodiments, the first polynucleotide and the third polynucleotide are contained within a polynucleotide sequence with at least 99% identity to SEQ ID NO: 33. In some embodiments, the first polynucleotide and the third polynucleotide are contained within the polynucleotide sequence according to SEQ ID NO: 33.

TABLE 3
First Polynucleotide Constructs

| | SEQ ID |
|---|---|
| Description | NO: |
| NFAT inducible IL-18 receptor alpha, 6XFOXP3NFAT | 26 |
| mCMVIL18receptor alpha | |
| NFAT inducible IL-18 receptor alpha, 3XFOXP3NFAT | 27 |
| mCMVIL18receptor alpha | |
| NFAT nducible IL-18 receptor alpha, 6XIL2NFAT mCMVIL18 | 28 |
| receptor alpha | |
| NFAT inducible IL-18 receptor alpha, 3XIL2NFAT | 29 |
| mCMVIL18receptor alpha | |
| NFAT inducible IL-18 receptor alpha and beta, | 30 |
| 6XFOXP3NFAT mCMVIL 18 receptor alpha-T2A-IL18Rb | |
| NFAT inducible IL-18 receptor alpha and beta, 3XFOXP3NFATmCMVIL18 | 31 |
| receptor alpha-T2A-IL18Rb | |
| NFAT inducible IL-18 receptor alpha and beta, | 32 |
| 6XIL2NFAT mCMVIL18 receptor alpha-T2A-IL18Rb | |
| NFAT inducible IL-18 receptor alpha and beta, | 33 |
| 3XIL2NFAT mCMVIL18 receptor alpha-T2A-IL18Rb | |

Second Polynucleotide that Encodes a Chimeric Antigen Receptor (CAR) Amino Acid Sequence

### CAR Targeting Moiety

In some embodiments, the CAR amino acid sequence comprises a targeting moiety. In some embodiments, the targeting moiety binds to MUC1 or MUC1*. n some embodiments, the targeting moiety binds to MUC1*. In some embodiments, the targeting moiety bind to the amino acid sequence according to SEQ ID NO: 35. In some embodiments, the targeting moiety bind to the amino acid sequence according to SEQ ID NO: 36.

In some embodiments, the targeting moiety comprises a heavy chain variable domain comprising heavy chain complementarity determining regions (CDR)s HC-CDR1, HC-CDR2, and HC-CDR3 and the targeting moiety comprises a light chain variable domain comprising light chain CDRs LC-CDR1, LC-CDR2, and LC-CDR3. In some embodiments, the HC-CDR1, HC-CDR2, and HC-CDR3 and the LC-CDR1, LC-CDR2, and LC-CDR3 comprise the amino acid sequences according to: HC-CDR1: SEQ ID NO: 37; HC-CDR2: SEQ ID NO: 38; HC-CDR3: SEQ ID NO: 39; LC-CDR1: SEQ ID NO: 40; LC-CDR2: SEQ ID NO: 41; and LC-CDR3: SEQ ID NO: 42. In some embodiments, the heavy chain variable domain comprises an amino acid sequence with at least 85% sequence identity to SEQ ID NO: 43 and the light chain variable domain comprises an amino acid sequence with at least 85% sequence identity to SEQ ID NO: 44. In some embodiments, the heavy chain variable domain comprises an amino acid sequence with at least 90% sequence identity to SEQ ID NO: 43 and the light chain variable domain comprises an amino acid sequence with at least 90% sequence identity to SEQ ID NO: 44. In some embodiments, the heavy chain variable domain comprises an amino acid sequence with at least 95% sequence identity to SEQ ID NO: 43 and the light chain variable domain comprises an amino acid sequence with at least 95% sequence identity to SEQ ID NO: 44. In some embodiments, the heavy chain variable domain comprises an amino acid sequence with at least 99% sequence identity to SEQ ID NO: 43 and the light chain variable domain comprises an amino acid sequence with at least 99% sequence identity to SEQ ID NO: 44. In some embodiments, the heavy chain variable domain comprises the amino acid sequence of SEQ ID NO: 43 and the light chain variable domain comprises the amino acid sequence of SEQ ID NO: 44. In some embodiments, the targeting moiety comprises a single chain variable fragment (scFv). In some embodiments, the scFv comprises an amino acid sequence with at least 85% sequence identity to SEQ ID NO: 45. In some embodiments, the scFv comprises an amino acid sequence with at least 90% sequence identity to SEQ ID NO: 45. In some embodiments, the scFv comprises an amino acid sequence with at least 95% sequence identity to SEQ ID NO: 45. In some embodiments, the scFv comprises an amino acid sequence with at least 99% sequence identity to SEQ ID NO: 45. In some embodiments, the scFv comprises the amino acid sequence of SEQ ID NO: 45.

In some embodiments, the HC-CDR1, HC-CDR2, and HC-CDR3 and the LC-CDR1, LC-CDR2, and LC-CDR3 comprise the amino acid sequences according to: HC-CDR1: SEQ ID NO: 46; HC-CDR2: SEQ ID NO: 47; HC-CDR3: SEQ ID NO: 48; LC-CDR1: SEQ ID NO: 49; LC-CDR2: SEQ ID NO: 50; and LC-CDR3: SEQ ID NO: 51. In some embodiments, the heavy chain variable domain comprises an amino acid sequence with at least 85% sequence identity to SEQ ID NO: 52 and the light chain variable domain comprises an amino acid sequence with at least 85% sequence identity to SEQ ID NO: 53. In some embodiments, the heavy chain variable domain comprises an amino acid sequence with at least 90% sequence identity to SEQ ID NO: 52 and the light chain variable domain comprises an amino acid sequence with at least 90% sequence identity to SEQ ID NO: 53. In some embodiments, the heavy chain variable domain comprises an amino acid sequence with at least 95% sequence identity to SEQ ID NO: 52 and the light chain variable domain comprises an amino acid sequence with at least 95% sequence identity to SEQ ID NO: 53. In some embodiments, the heavy chain variable domain comprises an amino acid sequence with at least 99% sequence identity to SEQ ID NO: 52 and the light chain variable domain comprises an amino acid sequence with at least 99% sequence identity to SEQ ID NO: 53. In some embodiments, the heavy chain variable domain comprises the amino acid sequence of SEQ ID NO: 52 and the light chain variable domain comprises the amino acid sequence of SEQ ID NO: 53. In some embodiments, the targeting moiety comprises a single chain variable fragment (scFv). In some embodiments, the scFv comprises an amino acid sequence with at least 85% sequence identity to SEQ ID NO: 54. In some embodiments, the scFv comprises an amino acid sequence with at least 90% sequence identity to SEQ ID NO: 54. In some embodiments, the scFv comprises an amino acid sequence with at least 95% sequence identity to SEQ ID NO: 54. In some embodiments, the scFv comprises an amino acid sequence with at least 99% sequence identity to SEQ ID NO: 54. In some embodiments, the scFv comprises the amino acid sequence of SEQ ID NO: 54.

In some embodiments, the HC-CDR1, HC-CDR2, and HC-CDR3 and the LC-CDR1, LC-CDR2, and LC-CDR3 comprise the amino acid sequences according to: HC-CDR1: SEQ ID NO: 55; HC-CDR2: SEQ ID NO: 56; HC-CDR3: SEQ ID NO: 57; LC-CDR1: SEQ ID NO: 58; LC-CDR2: SEQ ID NO: 59; and LC-CDR3: SEQ ID NO:60. In some embodiments, the heavy chain variable domain comprises an amino acid sequence with at least 85% sequence identity to SEQ ID NO: 61 and the light chain variable domain comprises an amino acid sequence with at least 85% sequence identity to SEQ ID NO: 62. In some embodiments, the heavy chain variable domain comprises an amino acid sequence with at least 90% sequence identity to SEQ ID NO: 61 and the light chain variable domain comprises an amino acid sequence with at least 90% sequence identity to SEQ ID NO: 62. In some embodiments, the heavy chain variable domain comprises an amino acid sequence with at least 95% sequence identity to SEQ ID NO: 61 and the light chain variable domain comprises an amino acid sequence with at least 95% sequence identity to SEQ ID NO: 62. In some embodiments, the heavy chain variable domain comprises an amino acid sequence with at least 99% sequence identity to SEQ ID NO: 61 and the light chain variable domain comprises an amino acid sequence with at least 99% sequence identity to SEQ ID NO: 62. In some embodiments, the heavy chain variable domain comprises the amino acid sequence of SEQ ID NO: 61 and the light chain variable domain comprises the amino acid sequence of SEQ ID NO: 62. In some embodiments, the targeting moiety comprises a single chain variable fragment (scFv). In some embodiments, the scFv comprises an amino acid sequence with at least 85% sequence identity to SEQ ID NO: 63. In some embodiments, the scFv comprises an amino acid sequence with at least 90% sequence identity to SEQ ID NO: 63. In some embodiments, the scFv comprises an amino acid sequence with at least 95% sequence identity to SEQ ID NO: 63. In some embodiments, the scFv comprises an amino acid sequence with at least 99% sequence identity to SEQ ID NO: 63. In some embodiments, the scFv comprises the amino acid sequence of SEQ ID NO: 63.

### Hinge

In some embodiments, the CAR amino acid sequence comprises a hinge amino acid sequence. In some embodiments, the hinge amino acid sequence comprises a CD8 amino acid sequence. In some embodiments, the hinge amino acid sequence comprises an amino acid sequence with at least 90% sequence identity to SEQ ID NO: 64. In some embodiments, the hinge amino acid sequence comprises the amino acid sequence of SEQ ID NO: 64. In some embodiments, the hinge amino acid sequence comprises a CD28 amino acid sequence. In some embodiments, the hinge amino acid sequence comprises an amino acid sequence with at least 90% sequence identity to SEQ ID NO: 65. In some embodiments, the hinge amino acid sequence comprises the amino acid sequence of SEQ ID NO: 65.

In some embodiments, the hinge comprises a sequence from a human protein. In some embodiments, the hinge is a human immunoglobulin hinge such as an IgG4 hinge or an IgD linker. In some embodiments, the hinge comprises a repeat of Glycines and Serines. In some embodiments, the hinge comprises a KIR2DS2 hinge.

### Transmembrane

In some embodiments, the CAR amino acid sequence comprises a transmembrane amino acid sequence. In some embodiments, the transmembrane amino acid sequence comprises a CD8 amino acid sequence. In some embodiments, the transmembrane amino acid sequence comprises an amino acid sequence with at least 90% sequence identity to SEQ ID NO: 66. In some embodiments, the transmembrane amino acid sequence comprises the amino acid sequence of SEQ ID NO: 66. In some embodiments, the transmembrane amino acid sequence comprises a CD28 amino acid sequence. In some embodiments, the transmembrane amino acid sequence comprises an amino acid sequence with at least 90% sequence identity to SEQ ID NO: 67. In some embodiments, the transmembrane amino acid sequence comprises the amino acid sequence of SEQ ID NO: 67.

In some embodiments, the transmembrane amino acid comprises a transmembrane amino acid from CD8, CD3ζ, CD4, CD28, 41-BB, or OX40.

### Co-Stimulatory

In some embodiments, the CAR amino acid sequence comprises a co-stimulatory amino acid sequence. In some embodiments, the co-stimulatory amino acid sequence comprises a 41-BB amino acid sequence. In some embodiments, the co-stimulatory amino acid sequence comprises an amino acid sequence with at least 90% sequence identity to SEQ ID NO: 68. In some embodiments, the co-stimulatory amino acid sequence comprises the amino acid sequence of SEQ ID NO: 68. In some embodiments, the co-stimulatory amino acid sequence comprises a CD28 amino acid sequence. In some embodiments, the co-stimulatory amino acid sequence comprises an amino acid sequence with at least 90% sequence identity to SEQ ID NO: 69. In some embodiments, the co-stimulatory amino acid sequence comprises the amino acid sequence of SEQ ID NO: 69.

In some embodiments, the co-stimulatory amino acid sequence comprises an amino acid sequence from CD27, CD28, 4-1BB, OX40, CD30, CD40, ICAm-1, LFA-1, ICOS, CD2, CD5, CD7 and Fc receptor gamma domain.

### Signaling

In some embodiments, the CAR amino acid sequence comprises a signaling amino acid sequence. In some embodiments, the signaling amino acid sequence comprises a CD3 amino acid sequence. In some embodiments, the signaling amino acid sequence comprises an amino acid sequence with at least 85% sequence identity to SEQ ID NO: 70. In some embodiments, the signaling amino acid sequence comprises an amino acid sequence with at least 90% sequence identity to SEQ ID NO: 70. In some embodiments, the signaling amino acid sequence comprises an amino acid sequence with at least 95% sequence identity to SEQ ID NO: 70. In some embodiments, the signaling amino acid sequence comprises an amino acid sequence with at least 99% sequence identity to SEQ ID NO: 70. In some embodiments, the signaling amino acid sequence comprises the amino acid sequence of SEQ ID NO: 70. In some embodiments, the signaling amino acid sequence comprises a cytoplasmic region comprising a CD3 intracellular signaling domain comprising a mutation in an immunoreceptor tyrosine-based activation motif (ITAM). In some embodiments, the mutation in the ITAM comprises a point mutation at a tyrosine residue. In some embodiments, the tyrosine residue is mutated to a phenylalanine residue. In some embodiments, the mutation in the ITAM comprises a mutation to more than one ITAM of the CD3ζ intracellular signaling domain. In some embodiments, the mutation to more than one ITAM of the CD3ζ intracellular signaling domain comprises mutations to two ITAMs. In some embodiments, the two ITAMs comprises the second and third ITAM of the CD3 intracellular signaling domain. In some embodiments, the mutation in the ITAM comprises a single point mutation in at least two ITAMs of the CD3ζ intracellular signaling domain. In some embodiments, the mutation in the ITAM comprises two point mutations at two tyrosine residues wherein the two tyrosine residues are mutated to phenylalanine residues. In some embodiments, the signaling amino acid sequence comprises an amino acid sequence with at least 85% sequence identity to SEQ ID NO: 71. In some embodiments, the signaling amino acid sequence comprises an amino acid sequence with at least 90% sequence identity to SEQ ID NO: 71. In some embodiments, the signaling amino acid sequence comprises an amino acid sequence with at least 95% sequence identity to SEQ ID NO: 71. In some embodiments, the signaling amino acid sequence comprises an amino acid sequence with at least 99% sequence identity to SEQ ID NO: 71. In some embodiments, the signaling amino acid sequence comprises the amino acid sequence of SEQ ID NO: 71.

In some embodiments, the signaling amino acid sequence comprises an amino acid sequence from CD3ζ, common FcR gamma (FCER1G), Fc gamma RIIa, FcR beta (Fc Epsilon R1b), CD3 gamma, CD3 delta, CD3 epsilon, CD5, CD22, CD79a, CD79b, CD278 (also known as “ICOS”), FcεRI, CD66d, DAP10, and DAP12.

TABLE 4
CAR Components

| | SEQ ID |
|---|---|
| Description | NO: |
| CD8 leader sequence | 34 |
| PSMGFR peptide. | 35 |
| N delta 10 portion of PSMGFR | 36 |
| Targeting Moiety | |
| C2 HC-CDR1 | 37 |
| C2 HC-CDR2 | 38 |
| C2 HC-CDR3 | 39 |
| C2 LC-CDR1 | 40 |
| C2 LC-CDR2 | 41 |
| C2 LC-CDR3 | 42 |
| C2 heavy chain variable domain | 43 |
| C2 light chain variable domain | 44 |
| C2 scFv | 45 |
| E6 HC-CDR1 | 46 |
| E6 HC-CDR2 | 47 |
| E6 HC-CDR3 | 48 |
| E6 LC-CDR1 | 49 |
| E6 LC-CDR2 | 50 |
| E6 LC-CDR3 | 51 |
| E6 heavy chain variable domain | 52 |
| E6 light chain variable domain | 53 |
| E6 scFv | 54 |
| 20A10 HC-CDR1 | 55 |
| 20A10 HC-CDR2 | 56 |
| 20A10 HC-CDR3 | 57 |
| 20A10 LC-CDR1 | 58 |
| 20A10 LC-CDR2 | 59 |
| 20A10 LC-CDR3 | 60 |
| 20A10 heavy chain variable domain | 61 |
| 20A10 light chain variable domain | 62 |
| 20A10 scFv | 63 |
| Hinge | |
| CD8 hinge amino acid sequence | 64 |
| CD28 hinge amino acid sequence | 65 |
| Transmembrane | |
| CD8 Transmembrane amino acid sequence | 66 |
| CD28 Transmembrane amino acid sequence | 67 |
| Co-stimulatory | |
| 41BB Costimulatory amino acid sequence | 68 |
| CD28 Costimulatory amino acid sequence | 69 |
| Signaling | |
| CD3ζ Signaling amino acid sequence. | 70 |
| CD3ζ 1XX Signaling amino acid sequence. | 71 |

### Car Constructs

In some embodiments, the CAR amino acid sequence comprises a hinge amino acid sequence according to SEQ ID NO: 65, a transmembrane amino acid sequence according to SEQ ID NO: 67, a co-stimulatory amino acid sequence according to SEQ ID NO: 69, and a signaling amino acid sequence according to SEQ ID NO:70. In some embodiments, the CAR amino acid sequence comprises a hinge amino acid sequence according to SEQ ID NO: 64, a transmembrane amino acid sequence according to SEQ ID NO: 66, a co-stimulatory amino acid sequence according to SEQ ID NO: 68, and a signaling amino acid sequence according to SEQ ID NO: 71. In some embodiments, the CAR amino acid sequence comprises a hinge amino acid sequence according to SEQ ID NO: 65, a transmembrane amino acid sequence according to SEQ ID NO: 67, a co-stimulatory amino acid sequence according to SEQ ID NO: 69, and a signaling amino acid sequence according to SEQ ID NO:71. In some embodiments, the CAR amino acid sequence comprises an amino acid sequence with at least 85% sequence identity to any one of SEQ ID NO: 72-83. In some embodiments, the CAR amino acid sequence comprises an amino acid sequence with at least 90% sequence identity to any one of SEQ ID NO: 72-83. In some embodiments, the CAR amino acid sequence comprises an amino acid sequence with at least 95% sequence identity to any one of SEQ ID NO: 72-83. In some embodiments, the CAR amino acid sequence comprises an amino acid sequence with at least 99% sequence identity to any one of SEQ ID NO: 72-83. In some embodiments, the CAR amino acid sequence comprises the amino acid sequence of any one of SEQ ID NO: 72-83. In some embodiments, the second polynucleotide comprises the polynucleotide sequence with at least 85% identity to any one of SEQ ID NO: 84-95. In some embodiments, the second polynucleotide comprises the polynucleotide sequence with at least 90% identity to any one of SEQ ID NO: 84-95. In some embodiments, the second polynucleotide comprises the polynucleotide sequence with at least 95% identity to any one of SEQ ID NO: 84-95. In some embodiments, the second polynucleotide comprises the polynucleotide sequence with at least 99% identity to any one of SEQ ID NO: 84-95. In some embodiments, the second polynucleotide comprises the polynucleotide sequence according to any one of SEQ ID NO: 84-95.

Disclosed herein are compositions comprising the second polynucleotide of any of the above embodiments, wherein the second polynucleotide further comprises a leader sequence. In some embodiments, the leader sequence increases uptake of the second polynucleotide into an immune cell. In some embodiments, the leader sequence increases uptake of the second polynucleotide into a nucleus of an immune cell. In some embodiments, the second polynucleotide encodes an amino acid sequence according to SEQ ID NO: 34.

TABLE 5
Listing of CAR constructs

| | | SEQ ID | |
|---|---|---|---|
| N-term- | | NO: | SEQ ID |
| Targeting | hinge-transmembrane-co- | (amino | NO: |
| Moiety | stimulatory-signaling-C-term | acid) | (nucleotide) |
| C2 | CD8-CD8-41BB CD3 | 72 | 84 |
| E6 | CD8-CD8-41BB CD3 | 73 | 85 |
| 20A10N | CD8-CD8-41BB CD3 | 74 | 86 |
| C2 | CD28-CD28-CD28CD3 | 75 | 87 |
| E6 | CD28-CD28-CD28CD3 | 76 | 88 |
| 20A10N | CD28-CD28-CD28CD3 | 77 | 89 |
| C2 | CD8-CD8-41BB 1XX | 78 | 90 |
| E6 | CD8-CD8-41BB 1XX | 79 | 91 |
| 20A10N | CD8-CD8-41BB 1XX | 80 | 92 |
| C2 | CD28-CD28-CD28 1XX | 81 | 93 |
| E6 | CD28-CD28-CD28 1XX | 82 | 94 |
| 20A10N | CD28-CD28-CD28 1XX | 83 | 95 |

### Vectors

In some embodiments, the first polynucleotide and the second polynucleotide are on the same vector. In some embodiments, the first polynucleotide and the second polynucleotide are on the same vector and the vector comprises a polynucleotide sequence of at least 85% identity to any one of SEQ ID NOs: 96-190. In some embodiments, the first polynucleotide and the second polynucleotide are on the same vector and the vector comprises a polynucleotide sequence of at least 90% identity to any one of SEQ ID NOs: 96-190. In some embodiments, the first polynucleotide and the second polynucleotide are on the same vector and the vector comprises a polynucleotide sequence of at least 95% identity to any one of SEQ ID NOs: 96-190. In some embodiments, the first polynucleotide and the second polynucleotide are on the same vector and the vector comprises a polynucleotide sequence of at least 99% identity to any one of SEQ ID NOs: 96-190. In some embodiments, the first polynucleotide and the second polynucleotide are on the same vector and the vector comprises a polynucleotide sequence of any one of SEQ ID NOs: 96-190. In some embodiments, the first polynucleotide and the second polynucleotide are on separate vectors.

In some embodiments, the vector can be a viral vector. As can be appreciated by one of skill in the art, the term “viral vector” can refer either to a nucleic acid molecule (e.g, a transfer plasmid) that includes virus-derived nucleic acid elements that generally facilitate transfer of the nucleic acid molecule or integration into the genome of a cell or to a viral particle that mediates nucleic acid transfer. Viral particles generally include various viral components and sometimes also host cell components in addition to nucleic acid(s). The term viral vector may refer either to a virus or viral particle capable of transferring a nucleic acid into a cell or to the transferred nucleic acid itself. Viral vectors and transfer plasmids contain structural and/or functional genetic elements that are primarily derived from a virus. In some embodiments, the vector is a vector derived from a lentivirus, an adeno virus, an adeno-associated virus, a baculovirus, or a retrovirus. The term “retroviral vector” refers to a viral vector or plasmid containing structural and functional genetic elements, or portions thereof, that are primarily derived from a retrovirus. The term “lentiviral vector” refers to a viral vector or plasmid containing structural and functional genetic elements, or portions thereof, including LTRs that are primarily derived from a lentivirus, which is a genus of retrovirus.

In some embodiments, the polynucleotides disclosed herein can be optimized for expression in the host cell of interest. For example, the G-C content of the sequence can be adjusted to average levels for a given cellular host, as calculated by reference to known genes expressed in the host cell. Methods for codon usage optimization are known in the art. Codon usages within the coding sequence of the chimeric receptor disclosed herein can be optimized to enhance expression in the host cell, such that about 1%, about 5%, about 10%, about 25%, about 50%, about 75%, or up to 100% of the codons within the coding sequence have been optimized for expression in a particular host cell.

The polynucleotides provided can contain naturally occurring sequences, or sequences that differ from those that occur naturally, but, due to the degeneracy of the genetic code, encode the same polypeptide, e.g., antibody for the ECD. These nucleic acid molecules can consist of RNA or DNA (for example, genomic DNA, cDNA, or synthetic DNA, such as that produced by phosphoramidite-based synthesis), or combinations or modifications of the nucleotides within these types of nucleic acids. In addition, the nucleic acid molecules can be double-stranded or single-stranded (e.g, either a sense or an antisense strand).

The polynucleotides disclosed herein are not limited to sequences that encode polypeptides (e.g, antibodies for the ECD); some or all of the non-coding sequences that lie upstream or downstream from a coding sequence (e.g, the coding sequence of a chimeric receptor) can also be included. Those of ordinary skill in the art of molecular biology are familiar with routine procedures for isolating nucleic acid molecules. They can, for example, be generated by treatment of genomic DNA with restriction endonucleases, or by performance of the polymerase chain reaction (PCR). In the event the nucleic acid molecule is a ribonucleic acid (RNA), molecules can be produced, for example, by in vitro transcription.

TABLE 6
Example Vector Sequences

| Description | SEQ ID NO: |
|---|---|
| CAR C2 CD8-CD8-41 BBCD3 6XFOXP3NFAT mCMVIL18Ralpha | 96 |
| CAR C2 CD8-CD8-41 BBCD3 3XFOXP3NFAT mCMVIL18Ralpha | 97 |
| CAR C2 CD8-CD8-41 BBCD3 6XIL2NFAT mCMVIL18Ralpha | 98 |
| CAR C2 CD8-CD8-41 BBCD3 3XIL2NFAT mCMVIL18Ralpha | 99 |
| CAR C2 CD28-CD28-CD28 CD3 6XFOXP3NFATm CMVIL18Ralpha | 100 |
| CAR C2 CD28-CD28-CD28 CD3 3XFOXP3NFATm CMVIL18Ralpha | 101 |
| CAR C2 CD28-CD28-CD28 CD3 6XIL2NFATm CMVIL18Ralpha | 102 |
| CAR C2 CD28-CD28-CD28 CD3 3XIL2NFAT mCMVIL18Ralpha | 103 |
| CAR C2 CD28-CD28-CD28 1XX 6XFOXP3NFAT mCMVIL18Ralpha | 104 |
| CAR C2 CD28-CD28-CD28 1XX 3XFOXP3NFAT mCMVIL18Ralpha | 105 |
| CAR C2 CD28-CD28-CD28 1XX 6XIL2NFAT mCMVIL18Ralpha | 106 |
| CAR C2 CD28-CD28-CD28 1XX 3XIL2NFAT mCMVIL18Ralpha | 107 |
| CAR C2 CD8-CD8-41BB 1XX 6XFOXP3NFAT mCMVIL18Ralpha | 168 |
| CAR C2 CD8-CD8-41BB 1XX 3XFOXP3NFAT mCMVIL18Ralpha | 169 |
| CAR C2 CD8-CD8-41BB 1XX 6XIL12NFAT mCMVIL18Ralpha | 170 |
| CAR C2 CD8-CD8-41BB 1XX 3XIL12NFAT mCMVIL18Ralpha | 171 |
| CAR E6 CD8-CD8-41BB CD3 6XFOXP3NFAT mCMVIL18Ralpha | 108 |
| CAR E6 CD8-CD8-41BB CD3 3XFOXP3NFAT mCMVIL18Ralpha | 109 |
| CAR E6 CD8-CD8-41BB CD3 6XIL2NFAT mCMVIL18Ralpha | 110 |
| CAR E6 CD8-CD8-41BB CD3 3XIL2NFAT mCMVIL18Ralpha | 111 |
| CAR E6 CD28-CD28-CD28 CD3 6XFOXP3NFAT mCMVIL18Ralpha | 112 |
| CAR E6 CD28-CD28-CD28 CD3 3XFOXP3NFA TmCMVIL18Ralpha | 113 |
| CAR E6 CD28-CD28-CD28 CD3 6XIL2NFAT mCMVIL18Ralpha | 114 |
| CAR E6 CD28-CD28-CD28 CD3 3XIL2NFAT mCMVIL18Ralpha | 115 |
| CAR E6 CD28-CD28-CD28 1XX 6XFOXP3NFAT mCMVIL18Ralpha | 116 |
| CAR E6 CD28-CD28-CD28 1XX3XFOXP3NFAT mCMVIL18Ralpha | 117 |
| CAR E6 CD28-CD28-CD28 1XX 6XIL2NFAT mCMVIL18Ralpha | 118 |
| CAR E6 CD28-CD28-CD28 1XX3XIL2NFAT mCMVIL18Ralpha | 119 |
| CAR E6 CD8-CD8-41BB1XX 6XFOXP3NFAT mCMVIL18Ralpha | 172 |
| CAR E6 CD8-CD8-41BB1XX 3XFOXP3NFAT mCMVIL18Ralpha | 173 |
| CAR E6 CD8-CD8-41BB1XX 6XIL12NFAT mCMVIL18Ralpha | 174 |
| CAR E6 CD8-CD8-41BB1XX 3XIL12NFAT mCMVIL18Ralpha | 175 |
| CAR 20A10N CD8-CD8-41BB CD3 6XFOXP3NFAT mCMVIL18Ralpha | 120 |
| CAR 20A10N CD8-CD8-41BB CD3 3XFOXP3NFAT mCMVIL18Ralpha | 121 |
| CAR 20A10N CD8-CD8-41BB CD3 6XIL2NFAT mCMVIL18Ralpha | 122 |
| CAR 20A10N CD8-CD8-41BB CD3 3XIL2NFAT mCMVIL18Ralpha | 123 |
| CAR 20A10N CD28-CD28-CD28 CD3 6XFOXP3NFAT mCMVIL18Ralpha | 124 |
| CAR 20A10N CD28-CD28-CD28 CD3 3XFOXP3NFAT mCMVIL18Ralpha | 125 |
| CAR 20A10N CD28-CD28-CD28 CD3 6XIL2NFAT mCMVIL18Ralpha | 126 |
| CAR 20A10N CD28-CD28-CD28 CD3 3XIL2NFAT mCMVIL18 Ralpha | 127 |
| CAR 20A10N CD28-CD28-CD28 1XX 6XFOXP3NFAT mCMVIL18Ralpha | 128 |
| CAR 20A10N CD28-CD28-CD28 1XX3XFOXP3NFAT mCMVIL18Ralpha | 129 |
| CAR 20A10N CD28-CD28-CD28 1XX 6XIL2NFAT mCMVIL18Ralpha | 130 |
| CAR 20A10N CD28-CD28-CD28 1XX 3XIL2NFAT mCMVIL18Ralpha | 131 |
| CAR C2 CD8-CD8-41BB CD3 6XFOXP3NFAT mCMVIL18Ralpha-T2A-IL18Rb | 132 |
| CAR C2 CD8-CD8-41BB CD3 3XFOXP3NFAT mCMVIL18Ralpha-T2A-IL18Rb | 133 |
| CAR C2 CD8-CD8-41BB CD3 6XIL2NFAT mCMVIL18Ralpha-T2A-IL18Rb | 134 |
| CAR C2 CD8-CD8-41BB CD3 3XIL2NFAT mCMVIL18Ralpha-T2A-IL18Rb | 135 |
| CAR C2 CD28-CD28-CD28 CD3 6XFOXP3NFAT mCMVIL18Ralpha-T2A-IL18Rb | 136 |
| CAR C2 CD28-CD28-CD28 CD3 3XFOXP3NFAT mCMVIL18Ralpha-T2A-IL18Rb | 137 |
| CAR C2 CD28-CD28-CD28 CD3 6XIL2NFAT mCMVIL18Ralpha-T2A-IL18Rb | 138 |
| CAR C2 CD28-CD28-CD28 CD3 3XIL2NFAT mCMVIL18Ralpha-T2A-IL18Rb | 139 |
| CAR C2 CD28-CD28-CD28 1XX 6XFOXP3NFAT mCMVIL18Ralpha-T2A-IL18Rb | 140 |
| CAR C2 CD28-CD28-CD28 1XX 3XFOXP3NFAT mCMVIL18Ralpha-T2A-IL18Rb | 141 |
| CAR C2 CD28-CD28-CD28 1XX 6XIL2NFAT mCMVIL18Ralpha-T2A-IL18Rb | 142 |
| CAR C2 CD28-CD28-CD28 1XX 3XIL2NFAT mCMVIL18Ralpha-T2A-IL18Rb | 143 |
| CAR C2 CD8-CD8-41BB 1XX 6XFOXP3NFAT mCMVIL18Ralpha-T2A-IL18Rb | 176 |
| CAR C2 CD8-CD8-41BB 1XX 3XFOXP3NFAT mCMVIL18Ralpha-T2A-IL18Rb | 177 |
| CAR C2 CD8-CD8-41BB 1XX 6XIL12NFAT mCMVIL18Ralpha-T2A-IL18Rb | 178 |
| CAR C2 CD8-CD8-41BB 1XX 3XIL12NFAT mCMVIL18Ralpha-T2A-IL18Rb | 179 |
| CAR E6 CD8-CD8-41BB CD3 6XFOXP3NFAT mCMVIL18Ralpha-T2A-IL18Rb | 144 |
| CAR E6 CD8-CD8-41BB CD3 3XFOXP3NFAT mCMVIL18Ralpha-T2A-IL18Rb | 145 |
| CAR E6 CD8-CD8-41BB CD3 6XIL2NFAT mCMVIL18Ralpha-T2A-IL 18Rb | 146 |
| CAR E6 CD8-CD8-41BB CD3 3XIL2NFAT mCMVIL18Ralpha-T2A-IL18Rb | 147 |
| CAR E6 CD28-CD28-CD28 CD3 6XFOXP3NFAT mCMVIL18Ralpha-T2A-IL18Rb | 148 |
| CAR E6 CD28-CD28-CD28 CD3 3XFOXP3NFAT mCMVIL18Ralpha-T2A-IL18Rb | 149 |
| CAR E6 CD28-CD28-CD28 CD3 6XIL2NFAT mCMVIL18Ralpha-T2A-IL18Rb | 150 |
| CAR E6 CD28-CD28-CD28 CD3 3XIL2NFAT mCMVIL18Ralpha-T2A-IL18Rb | 151 |
| CAR E6 CD28-CD28-CD28 1XX 6XFOXP3NFAT mCMVIL18Ralpha-T2A-IL18Rb | 152 |
| CAR E6 CD28-CD28-CD28 1XX 3XFOXP3NFAT mCMVIL18Ralpha-T2A-IL18Rb | 153 |
| CAR E6 CD28-CD28-CD28 1XX 6XIL2NFAT mCMVIL18Ralpha-T2A-IL18Rb | 154 |
| CAR E6 CD28-CD28-CD28 1XX 3XIL2NFAT mCMVIL18Ralpha-T2A-IL18Rb | 155 |
| CAR E6 CD8-CD8-41BB 1XX 6XFOXP3NFAT mCMVIL18Ralpha-T2A-IL18Rb | 180 |
| CAR E6 CD8-CD8-41BB 1XX 3XFOXP3NFAT mCMVIL18Ralpha-T2A-IL18Rb | 181 |
| CAR E6 CD8-CD8-41BB 1XX 6XIL12NFAT mCMVIL18Ralpha-T2A-IL18Rb | 182 |
| CAR E6 CD8-CD8-41BB 1XX 3XIL12NFAT mCMVIL18Ralpha-T2A-IL18Rb | 183 |
| CAR 20A10N CD8-CD8-41BB CD3 6XFOXP3NFAT mCMVIL18Ralpha-T2A-IL18Rb | 156 |
| CAR 20A10N CD8-CD8-41BB CD3 3XFOXP3NFAT mCMVIL18Ralpha-T2A-IL18Rb | 157 |
| CAR 20A10N CD8-CD8-41BB CD3 6XIL2NFAT mCMVIL18Ralpha-T2A-IL18Rb | 158 |
| CAR 20A10N CD8-CD8-41BB CD3 3XIL2NFAT mCMVIL18Ralpha-T2A-IL18Rb | 159 |
| CAR 20A10N CD28-CD28-CD28 CD3 6XFOXP3NFAT mCMVIL18Ralpha-T2A-IL18Rb | 160 |
| CAR 20A10N CD28-CD28-CD28 CD3 3XFOXP3NFAT mCMVIL18Ralpha-T2A-IL18Rb | 161 |
| CAR 20A10N CD28-CD28-CD28 CD3 6XIL2NFAT mCMVIL18Ralpha-T2A-IL18Rb | 162 |
| CAR 20A10N CD28-CD28-CD28 CD3 3XIL2NFAT mCMVIL18Ralpha-T2A-IL18Rb | 163 |
| CAR 20A10N CD28-CD28-CD28 1XX 6XFOXP3NFAT mCMVIL18Ralpha-T2A- | 164 |
| IL18Rb | |
| CAR 20A10N CD28-CD28-CD28 1XX 3XFOXP3NFAT mCMVIL18Ralpha-T2A- | 165 |
| IL18Rb | |
| CAR 20A10N CD28-CD28-CD28 1XX 6XIL2NFAT mCMVIL18Ralpha-T2A-IL18Rb | 166 |
| CAR 20A10N CD28-CD28-CD28 1XX 3XIL2NFAT mCMVIL18Ralpha-T2A-IL18Rb | 167 |
| CAR 20A10N CD8-CD8-41BB 1XX 6XFOXP3NFAT mCMVIL18Ralpha | 184 |
| CAR 20A10N CD8-CD8-41BB 1XX 3XFOXP3NFAT mCMVIL18Ralpha | 185 |
| CAR 20A10N CD8-CD8-41BB 1XX 6XIL12NFAT mCMVIL18Ralpha | 186 |
| CAR 20A10N CD8-CD8-41BB 1XX 6XFOXP3NFAT mCMVIL18Ralpha-T2A-IL18Rb | 187 |
| CAR 20A10N CD8-CD8-41BB 1XX 3XFOXP3NFAT mCMVIL18Ralpha-T2A-IL18Rb | 188 |
| CAR 20A10N CD8-CD8-41BB 1XX 6XIL12NFAT mCMVIL18Ralpha-T2A-IL18Rb | 189 |
| CAR 20A10N CD8-CD8-41BB 1XX3 XIL12NFAT mCMVIL18Ralpha-T2A-IL18Rb | 190 |

### Immune Cells and Pharmaceutical Compositions

In some embodiments, the immune cell is a T cell. In some embodiments, the immune cell is autologous. In some embodiments, the immune cell is allogeneic.

In some embodiments, the cell is derived from a patient or from a donor. In some embodiments, the cell remains active at least 30 days after administration to a patient. In some embodiments, the cell remains active at least 60 days after administration to a patient. In some embodiments, the cell remains active at least 90 days after administration to a patient.

The polynucleotides of the present disclosure can be introduced into a cell (i.e., a host cell), such as a human T cell, to produce a recombinant cell containing the nucleic acid molecule. Accordingly, some embodiments of the disclosure relate to methods for making a recombinant cell, including (a) providing a host cell capable of protein expression; and transducing the provided host cell with a recombinant nucleic acid of the disclosure to produce a recombinant cell. Introduction of the polynucleotides of the disclosure into cells can be achieved by methods known to those skilled in the art such as, for example, viral infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like.

Accordingly, in some embodiments, the polynucleotides can be introduced into a host cell by viral or non-viral delivery vehicles known in the art to produce an engineered cell. For example, the nucleic acid molecule can be stably integrated in the host genome, or can be episomally replicating, or present in the recombinant host cell as a mini-circle expression vector for a stable or transient expression. Accordingly, in some embodiments disclosed herein, the nucleic acid molecule is maintained and replicated in the recombinant host cell as an episomal unit. In some embodiments, the nucleic acid molecule is stably integrated into the genome of the recombinant cell. Stable integration can be completed using classical random genomic recombination techniques or with more precise genome editing techniques such as using zinc-finger proteins (ZNF), guide RNA directed CRISPR/Cas9, DNA-guided endonuclease genome editing NgAgo (Nalronobacleriiim gregoryi Argonaute), or TALEN genome editing (transcription activator-like effector nucleases). In a non-limiting example, guide RNA direct CRISPR/Cas9 or TALEN genome editing may be applied targeting chimeric antigen receptor (CAR) to the T-cell receptor α constant (TRAC) locus, to enhance T-cell potency, re-expression of CAR following single or repeated exposure to antigen, delaying effector T-cell differentiation and exhaustion.

When introducing more than one nucleic acid sequence into a host cell, the efficiency of polynucleotide uptake decreases as the size of the polynucleotide or the number of individual nucleic acid sequences increases. A solution to this potential problem is to stably integrate the synthetic polynucleotide into the genome of the host. Site specific integration can be accomplished using gene editing tools such as DNA or RNA editing tools such as CRISPR/Cas9, TALEN, Sleeping Beauty, piggy Bac, Super piggy Bac, and the like to insert a synthetic nucleic acid sequence into the genome of the host cell. For example, the nucleic acid sequence encoding a CAR can be inserted into the T-cell receptor α constant (TRAC) locus. Nucleic acid sequences encoding an inducible cytokine receptor, such as IL-18Rα, can be site specifically inserted into the Foxp3 enhancer region of the host T cell. When more than one polynucleotide is intended to be inserted into a cell, the possibility exists that a single cell may only take up one of the polynucleotides. Polynucleotides encoding more than one species can be separated by self-cleaving sequences. The T2A sequence is a nucleic acid sequence that is cleaved after cellular internalization. Yet another method for introducing polynucleotides into a host cell involves in vivo manufacture. In this instance, a synthetic targeting moiety is attached to the polynucleotide. The targeting moiety selectively brings the polynucleotide to a target cell, wherein the moiety also facilitates uptake of the polynucleotide into the target cell. In some cases, the polynucleotide is an mRNA sequence. In some cases, the target cell is an immune cell. In some cases, the immune cell is a T cell or an NK cell.

The polynucleotides can be encapsulated in a viral capsid or a lipid nanoparticle, or can be delivered by viral or non-viral delivery means and methods known in the art, such as electroporation. For example, introduction of nucleic acids into cells may be achieved by viral transduction. In a non-limiting example, baculoviral virus or adeno-associated virus (AAV) can be engineered to deliver nucleic acids to target cells via viral transduction. Several AAV serotypes have been described, and all of the known serotypes can infect cells from multiple diverse tissue types. AAV is capable of transducing a wide range of species and tissues in vivo with no evidence of toxicity, and it generates relatively mild innate and adaptive immune responses.

Lentiviral-derived vector systems are also useful for nucleic acid delivery and gene therapy via viral transduction. Lentiviral vectors offer several attractive properties as gene-delivery vehicles, including: (i) sustained gene delivery through stable vector integration into host genome; (ii) the capability of infecting both dividing and non-dividing cells; (iii) broad tissue tropisms, including important gene- and cell-therapy-target cell types; (iv) no expression of viral proteins after vector transduction; (v) the ability to deliver complex genetic elements, such as polycistronic or intron-containing sequences; (vi) a potentially safer integration site profile; and (vii) a relatively easy system for vector manipulation and production.

In some embodiments, host cells can be genetically engineered (e.g, transduced or transformed or transfected) with, for example, a vector construct of the present application that can be, for example, a viral vector or a vector for homologous recombination that includes nucleic acid sequences homologous to a portion of the genome of the host cell, or can be an expression vector for the expression of the CAR polypeptides of interest.

As outlined above, some embodiments of the disclosure relate to various methods for making a recombinant cell, including (a) providing a host cell capable of protein expression; and transducing the provided host cell with a recombinant nucleic acid of the disclosure to produce a recombinant cell. Non-limiting exemplary embodiments of the disclosed methods for making a recombinant cell can further include one or more of the following features. In some embodiments, the host cell is obtained by leukapheresis performed on a sample obtained from a subject, and the cell is transduced ex vivo. In some embodiments, the recombinant nucleic acid is encapsulated in a viral capsid or a lipid nanoparticle. In some embodiments, the methods further include isolating and/or purifying the produced cells. Accordingly, the recombinant cells produced by the methods disclosed herein are also within the scope of the disclosure.

Techniques for transforming a wide variety of the above-mentioned host cells and species are known in the art and described in the technical and scientific literature. For example, DNA vectors can be introduced into eukaryotic cells via conventional transformation or transfection techniques. Suitable methods for transforming or transfecting cells can be found in Sambrook et al. (2012, supra) and other standard molecular biology laboratory manuals, such as, calcium phosphate transfection, DEAE-dextran mediated transfection, transfection, microinjection, cationic lipid-mediated transfection, electroporation, transduction, scrape loading, ballistic introduction, nucleoporation, hydrodynamic shock, and infection. In some embodiments, the nucleic acid molecule is introduced into a host cell by a transduction procedure, electroporation procedure, or a biolistic procedure. Accordingly, cell cultures including at least one recombinant cell as disclosed herein are also within the scope of this application.

Yet another method for introducing polynucleotides into a host cell involves in vivo manufacture. In this instance, a synthetic targeting moiety is attached to the polynucleotide. The targeting moiety selectively brings the polynucleotide to a target cell, wherein the moiety also facilitates uptake of the polynucleotide into the target cell. In some cases, the polynucleotide is an mRNA sequence. In some cases, the target cell is an immune cell. In some cases, the immune cell is a T cell or an NK cell.

In some embodiments, disclosed herein are pharmaceutical compositions comprising the immune cell of any of the above embodiments. In some embodiments, the cell is in a saline solution comprising human serum albumin. In some embodiments, the cell is in an infusible cryopreservation solution. In some embodiments, the infusible cryopreservation solution comprises one or more of sodium, potassium, magnesium, chloride, acetate, or gluconate. In some embodiments, the infusible cryopreservation solution comprises 140 mEq sodium, 5 mEq potassium, 3 mEq magnesium, 98 mEq chloride, 27 mEq acetate, and 23 mEq gluconate. In some embodiments, the infusible cryopreservation solution comprises human serum albumin (HSA) at a final concentration of 2.5%. In some embodiments, the infusible cryopreservation solution has a pH from 7.0 to 7.5. In some embodiments, the infusible cryopreservation solution has a pH of 7.4. In some embodiments, the infusible cryopreservation solution comprises dimethyl sulfoxide (DMSO). In some embodiments, the infusible cryopreservation solution comprises 5% (w/v) dimethyl sulfoxide (DMSO).

### Methods of Use

Disclosed herein are methods of treating cancer in a subject in need thereof comprises administering the immune cell of any of the above embodiments. In some embodiments, the cancer comprises a blood cancer. In some embodiments, the cancer comprises a solid tumor. In some embodiments, the cancer comprises breast cancer, lung cancer, prostate cancer, ovarian cancer, colorectal cancer, pancreatic cancer. In some embodiments, the cancer is a classified as a stage 1 cancer. In some embodiments, the cancer is a classified as a stage 2 cancer. In some embodiments, the cancer is a classified as a stage 3 cancer. In some embodiments, the cancer is characterized by a low antigen expressing cell. In some embodiments, the cancer is a classified as a stage 4 cancer. In some embodiments, the cancer is characterized by a high antigen expressing cell.

Disclosed herein are methods of increasing CAR T persistence in a subject in need thereof comprising administering the immune cell of any of the above embodiments.

Disclosed herein are methods of inhibiting cancer recurrence in a subject in need thereof comprising administering the immune cell of any of the above embodiments.

In some embodiments, the cancer expresses MUC1. In some embodiments, the cancer expresses MUC1*. In some embodiments, the cancer is MMP9 positive. In some embodiments, the cancer is breast cancer, colon cancer, prostate cancer, pancreatic cancer, or lung cancer. In some embodiments, the breast cancer is HER2−/ER+/PR−, or HER2−/ER+/PR+, or HER2−/ER−/PR+, or HER2+, or HER2+/PR+/ER−, HER2+/ER+/PR−, or triple negative breast cancer. In some embodiments, the breast cancer is HER2−/ER+/PR−. In some embodiments, the breast cancer is HER2−/ER+/PR+. In some embodiments, the breast cancer is HER2−/ER−/PR+. In some embodiments, the breast cancer is HER2+/PR+/ER−. In some embodiments, the breast cancer is HER2+/ER+/PR−. In some embodiments, the breast cancer is triple negative breast cancer.

In some embodiments, the method further comprises administration of an anti-cancer agent. In some embodiments, the anti-cancer agent comprises a cytotoxic agent. In some embodiments, the cytotoxic agent comprises a platinum-based agent or a taxane.

### Articles of Manufacture

In another aspect of the disclosure, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic.

The label or package insert indicates that the composition is used for treating the condition of choice. The article of manufacture in this embodiment of the disclosure may further comprise a package insert indicating that the compositions can be used to treat a particular condition.

Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### Definitions

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “antibody” is used in the broadest sense and covers fully assembled antibodies, antibody fragments that can bind antigen, for example, Fab, F(ab′)2, Fv, single chain antibodies (scFv), diabodies, antibody chimeras, hybrid antibodies, bispecific antibodies, and the like.

The term “complementarity determining region” or “CDR” is a segment of the variable region of an antibody that is complementary in structure to the epitope to which the antibody binds and is more variable than the rest of the variable region. Accordingly, a CDR is sometimes referred to as hypervariable region. A variable region comprises three CDRs. CDR peptides can be obtained by constructing genes encoding the CDR of an antibody of interest. Such genes are prepared, for example, by using the polymerase chain reaction to synthesize the variable region from RNA of antibody-producing cells. See, for example, Larrick et al., Methods: A Companion to Methods in Enzymology 2:106 (1991); Courtenay-Luck, “Genetic Manipulation of Monoclonal Antibodies,” in Monoclonal Antibodies: Production, Engineering and Clinical Application, Ritter et al. (eds.), pages 166-179 (Cambridge University Press 1995); and Ward et al., “Genetic Manipulation and Expression of Antibodies,” in Monoclonal Antibodies: Principles and Applications, Birch et al., (eds.), pages 137-185 (Wiley-Liss, Inc. 1995).

In some instances, the CDRs of an antibody are determined according to (i) the Kabat numbering system (Kabat et al. (197) Ann. NY Acad. Sci. 190:382-391 and, Kabat et al. (1991) Sequences of Proteins of Immunological Interest Fifth Edition, U.S. Department of Health and Human Services, NIH Publication No. 91-3242); or (ii) the Chothia numbering scheme, which will be referred to herein as the “Chothia CDRs” (see, e.g., Chothia and Lesk, 1987, J. Mol. Biol., 196:901-917; Al-Lazikani et al., 1997, J. Mol. Biol., 273:927-948; Chothia et al., 1992, J. Mol. Biol., 227:799-817; Tramontano A et al., 1990, J. Mol. Biol. 215 (1): 175-82; and U.S. Pat. No. 7,709,226); or (iii) the ImMunoGeneTics (IMGT) numbering system, for example, as described in Lefranc, M.-P., 1999, The Immunologist, 7:132-136 and Lefranc, M.-P. et al, 1999, Nucleic Acids Res., 27:209-212 (“IMGT CDRs”); or (iv) MacCallum et al, 1996, J. Mol. Biol., 262:732-745. See also, e.g., Martin, A., “Protein Sequence and Structure Analysis of Antibody Variable Domains,” in Antibody Engineering, Kontermann and Diibel, eds., Chapter 31, pp. 422-439, Springer-Verlag, Berlin (2001).

With respect to the Kabat numbering system, CDRs within an antibody heavy chain molecule are typically present at amino acid positions 31 to 35, which optionally can include one or two additional amino acids, following 35 (referred to in the Kabat numbering scheme as 35 A and 35B) (CDRI), amino acid positions 50 to 65 (CDR2), and amino acid positions 95 to 102 (CDR3). Using the Kabat numbering system, CDRs within an antibody light chain molecule are typically present at amino acid positions 24 to 34 (CDRI), amino acid positions 50 to 56 (CDR2), and amino acid positions 89 to 97 (CDR3). As is well known to those of skill in the art, using the Kabat numbering system, the actual linear amino acid sequence of the antibody variable domain can contain fewer or additional amino acids due to a shortening or lengthening of a FR and/or CDR and, as such, an amino acid's Kabat number is not necessarily the same as its linear amino acid number.

A “single-chain variable fragment (scFv)” is a fusion protein of the variable regions of the heavy (VH) and light chains (VL) of an antibody, connected with a short linker peptide of ten to about 25 amino acids. The linker is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. scFv antibodies are, e.g. described in Houston, J. S., Methods in Enzymol. 203 (1991) 46-96). In addition, antibody fragments comprise single chain polypeptides having the characteristics of a VH domain, namely being able to assemble together with a VL domain, or of a VL domain, namely being able to assemble together with a VH domain to a functional antigen binding site and thereby providing the antigen binding property of full length antibodies.

As used herein, the term “percent (%) amino acid sequence identity” or “percent (%) identity” with respect to a sequence is defined as the percentage of amino acid residues in a candidate sequence that are identical with the amino acid residues in the specific sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as EMBOSS MATCHER, EMBOSS WATER, EMBOSS STRETCHER, EMBOSS NEEDLE, EMBOSS LALIGN, BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

In some embodiments, the IL-18 receptor amino acid or the CAR amino acid comprise a modified amino acid or non-natural amino acid, or a modified non-natural amino acid, or a combination thereof. In some embodiments, the modified amino acid or a modified non-natural amino acid comprises a post-translational modification. In some embodiments the IL-18 receptor amino acid or the CAR amino acid comprise a modification including, but not limited to acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. Modifications are made anywhere to the IL-18 receptor amino acid or the CAR amino acid including the peptide backbone, the amino acid side chains, and the terminus.

The terms “individual(s)”, “subject(s)” and “patient(s)” are used interchangeably herein and refer to any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Embodiments

Embodiment 1. An immune cell comprising: a) a first polynucleotide that encodes an interleukin-18 (IL-18) receptor amino acid sequence that comprises an IL-18 receptor extracellular domain; and b) a second polynucleotide that encodes a chimeric antigen receptor (CAR) amino acid sequence.

Embodiment 2. The immune cell of embodiment 1, wherein the IL-18 receptor amino acid sequence comprises an IL-18 receptor alpha amino acid sequence.

Embodiment 3. The immune cell of any one of embodiments 1-2, wherein the IL-18 receptor extracellular domain comprises an amino acid sequence with at least 90% sequence identity to SEQ ID NO: 2.

Embodiment 4. The immune cell of any one of embodiments 1-3, wherein the IL-18 receptor extracellular domain comprises the amino acid sequence of SEQ ID NO: 2.

Embodiment 5. The immune cell of any one of embodiments 1-4, wherein the IL-18 receptor alpha amino acid sequence comprises a transmembrane IL-18 receptor alpha amino acid sequence.

Embodiment 6. The immune cell of embodiment 5, wherein the transmembrane IL-18 receptor alpha amino acid sequence comprises an amino acid sequence with at least 90% sequence identity to SEQ ID NO: 3.

Embodiment 7. The immune cell of any one of embodiments 5-6, wherein the transmembrane IL-18 receptor alpha amino acid sequence comprises the amino acid sequence of SEQ ID NO: 3.

Embodiment 8. The immune cell of any one of embodiments 2-7, wherein the IL-18 receptor alpha amino acid sequence comprises an intracellular IL-18 receptor alpha amino acid sequence.

Embodiment 9. The immune cell of embodiment 8, wherein the intracellular IL-18 receptor alpha amino acid sequence comprises an amino acid sequence with at least 90% sequence identity to SEQ ID NO: 4.

Embodiment 10. The immune cell of any one of embodiments 8-9, wherein the intracellular IL-18 receptor alpha amino acid sequence comprises the amino acid sequence of SEQ ID NO: 4.

Embodiment 11. The immune cell of any one of embodiments 2-10, wherein the IL-18 receptor alpha amino acid sequence comprises an amino acid sequence with at least 90% sequence identity to SEQ ID NO: 5.

Embodiment 12. The immune cell of any one of embodiments 2-11, wherein the IL-18 receptor alpha amino acid sequence comprises the amino acid sequence of SEQ ID NO: 5.

Embodiment 13. The immune cell of any one of embodiments 1-12, wherein the first polynucleotide comprises the polynucleotide sequence with at least 90% identity to SEQ ID NO: 6.

Embodiment 14. The immune cell of any one of embodiments 1-13, wherein the first polynucleotide comprises the polynucleotide sequence according to SEQ ID NO: 6.

Embodiment 15. The immune cell of embodiment 1, wherein the IL-18 receptor amino acid sequence comprises an IL-18 receptor beta amino acid sequence.

Embodiment 16. The immune cell of embodiment 15, wherein the IL-18 receptor extracellular domain comprises an amino acid sequence with at least 90% sequence identity to SEQ ID NO: 8.

Embodiment 17. The immune cell of any one of embodiments 15-16, wherein the IL-18 receptor extracellular domain comprises the amino acid sequence of SEQ ID NO: 8.

Embodiment 18. The immune cell of any one of embodiments 15-17, wherein the IL-18 receptor beta amino acid sequence comprises a transmembrane IL-18 receptor beta amino acid sequence.

Embodiment 19. The immune cell of embodiment 18, wherein the transmembrane IL-18 receptor beta amino acid sequence comprises an amino acid sequence with at least 90% sequence identity to SEQ ID NO: 9.

Embodiment 20. The immune cell of any one of embodiments 18-19, wherein the transmembrane IL-18 receptor beta amino acid sequence comprises the amino acid sequence of SEQ ID NO: 9.

Embodiment 21. The immune cell of any one of embodiments 15-20, wherein the IL-18 receptor beta amino acid sequence comprises an intracellular IL-18 receptor beta amino acid sequence.

Embodiment 22. The immune cell of embodiment 21, wherein the intracellular IL-18 receptor beta amino acid sequence comprises an amino acid sequence with at least 90% sequence identity to SEQ ID NO: 10.

Embodiment 23. The immune cell of any one of embodiments 21-22, wherein the intracellular IL-18 receptor beta amino acid sequence comprises the amino acid sequence of SEQ ID NO: 10.

Embodiment 24. The immune cell of any one of embodiments 15-23, wherein the IL-18 receptor beta amino acid sequence comprises an amino acid sequence with at least 90% sequence identity to SEQ ID NO: 11.

Embodiment 25. The immune cell of any one of embodiments 15-24, wherein the IL-18 receptor beta amino acid sequence comprises the amino acid sequence of SEQ ID NO: 11.

Embodiment 26. The immune cell of any one of embodiments 15-25, wherein the first polynucleotide comprises the polynucleotide sequence with at least 90% identity to SEQ ID NO: 12.

Embodiment 27. The immune cell of any one of embodiments 15-26, wherein the first polynucleotide comprises the polynucleotide sequence according to SEQ ID NO: 12.

Embodiment 28. The immune cell of any one of embodiments 1-27, wherein expression of the first polynucleotide is inducible.

Embodiment 29. The immune cell of any one of embodiments 1-28, wherein expression of the first polynucleotide is inducible when the CAR is stimulated by an antigen.

Embodiment 30. The immune cell of any one of embodiments 1-29, wherein expression of the first polynucleotide is operably linked to an inducible system.

Embodiment 31. The immune cell of embodiment 30, wherein the inducible system comprises a nuclear factor of activated T-cells (NFAT) response element.

Embodiment 32. The immune cell of embodiment 31, wherein the inducible system comprises 1 NFAT response element, 2 NFAT response elements, 3 NFAT response elements, 4 NFAT response elements, 5 NFAT response elements, or 6 NFAT response elements.

Embodiment 33. The immune cell of embodiment 31, wherein the NFAT response element comprises a FOXP3 NFAT response element.

Embodiment 34. The immune cell of embodiment 33, wherein the FOXP3 NFAT response element comprises the nucleotide sequence of SEQ ID NO: 13.

Embodiment 35. The immune cell of any one of embodiments 30-34, wherein the inducible system comprises a repeat of 3 FOXP3 NFAT response elements.

Embodiment 36. The immune cell of embodiment 35, wherein the repeat of 3 FOXP3 NFAT response elements comprises the nucleotide sequence of SEQ ID NO: 14.

Embodiment 37. The immune cell of any one of embodiments 30-34, wherein the inducible system comprises a repeat of 6 FOXP3 NFAT response elements.

Embodiment 38. The immune cell of embodiment 37, wherein the repeat of 6 FOXP3 NFAT response elements comprises the nucleotide sequence of SEQ ID NO: 15.

Embodiment 39. The immune cell of any one of embodiments 30-38, wherein the inducible system comprises a minimum CMV promoter (mCMV).

Embodiment 40. The immune cell of embodiment 39, wherein the minimum CMV promoter (mCMV) comprises the nucleotide sequence of SEQ ID NO: 16.

Embodiment 41. The immune cell of any one of embodiments 30-40, wherein the inducible system comprises the nucleotide sequence of SEQ ID NO: 17 or SEQ ID NO: 18.

Embodiment 42. The immune cell of embodiment 33, wherein the NFAT response element comprises a IL2 NFAT response element.

Embodiment 43. The immune cell of embodiment 42, wherein the IL2 NFAT response element comprises the nucleotide sequence of SEQ ID NO: 19.

Embodiment 44. The immune cell of any one of embodiments 30-43, wherein the inducible system comprises a repeat of 3 IL2 NFAT response elements.

Embodiment 45. The immune cell of embodiment 44, wherein the repeat of 3 IL2 NF AT response elements comprises the nucleotide sequence of SEQ ID NO: 20.

Embodiment 46. The immune cell of any one of embodiments 30-45, wherein the inducible system comprises a repeat of 6 IL2 NFAT response elements.

Embodiment 47. The immune cell of embodiment 46, wherein the repeat of 6 IL2 NF AT response elements comprises the nucleotide sequence of SEQ ID NO: 21.

Embodiment 48. The immune cell of any one of embodiments 30-47, wherein the inducible system comprises the nucleotide sequence of SEQ ID NO: 22 or SEQ ID NO: 23.

Embodiment 49. The immune cell of any one of embodiments 1-48, wherein the immune cell further comprises a third polynucleotide that encodes a second IL-18 receptor amino acid sequence that comprises an IL-18 receptor extracellular domain.

Embodiment 50. The immune cell of embodiment 49, wherein the second IL-18 receptor amino acid sequence comprises an IL-18 receptor alpha amino acid sequence.

Embodiment 51. The immune cell of any one of embodiments 49-50, wherein the second IL-18 receptor amino acid sequence comprises an amino acid sequence with at least 90% sequence identity to any one of SEQ ID NOs: 1-6.

Embodiment 52. The immune cell of any one of embodiments 49-51, wherein the second IL-18 receptor amino acid sequence comprises the amino acid sequence of any one of SEQ ID NOs: 1-6.

Embodiment 53. The immune cell of any one of embodiments 49-52, wherein the third polynucleotide comprises the polynucleotide sequence with at least 90% identity to any one of SEQ ID NO: 6.

Embodiment 54. The immune cell of any one of embodiments 49-53, wherein the third polynucleotide comprises the polynucleotide sequence according to any one of SEQ ID NO: 6.

Embodiment 55. The immune cell of any one of embodiments 49-54, wherein the second IL-18 receptor amino acid sequence comprises an IL-18 receptor beta amino acid sequence.

Embodiment 56. The immune cell of any one of embodiments 49-55, wherein the second IL-18 receptor amino acid sequence comprises an amino acid sequence with at least 90% sequence identity to any one of SEQ ID NOs: 7-11.

Embodiment 57. The immune cell of any one of embodiments 49-56, wherein the second IL-18 receptor amino acid sequence comprises the amino acid sequence of any one of SEQ ID NOs: 7-11.

Embodiment 58. The immune cell of any one of embodiments 49-57, wherein the third polynucleotide comprises the polynucleotide sequence with at least 90% identity to any one of SEQ ID NO: 12.

Embodiment 59. The immune cell of any one of embodiments 49-58, wherein the third polynucleotide comprises the polynucleotide sequence according to any one of SEQ ID NO: 12.

Embodiment 60. The immune cell of any one of embodiments 1-59, wherein the first polynucleotide comprises the polynucleotide sequence with at least 90% identity to SEQ ID NO: 26.

Embodiment 61. The immune cell of any one of embodiments 1-60, wherein the first polynucleotide comprises the polynucleotide sequence according to SEQ ID NO: 26.

Embodiment 62. The immune cell of any one of embodiments 1-61, wherein the first polynucleotide comprises the polynucleotide sequence with at least 90% identity to SEQ ID NO: 27.

Embodiment 63. The immune cell of any one of embodiments 1-62, wherein the first polynucleotide comprises the polynucleotide sequence according to SEQ ID NO: 27.

Embodiment 64. The immune cell of any one of embodiments 1-63, wherein the first polynucleotide comprises the polynucleotide sequence with at least 90% identity to SEQ ID NO: 28.

Embodiment 65. The immune cell of any one of embodiments 1-64, wherein the first polynucleotide comprises the polynucleotide sequence according to SEQ ID NO: 28.

Embodiment 66. The immune cell of any one of embodiments 1-65, wherein the first polynucleotide comprises the polynucleotide sequence with at least 90% identity to SEQ ID NO: 29.

Embodiment 67. The immune cell of any one of embodiments 1-66, wherein the first polynucleotide comprises the polynucleotide sequence according to SEQ ID NO: 29.

Embodiment 68. The immune cell of embodiment 49, wherein the first polynucleotide and the third polynucleotide are contained within a polynucleotide sequence with at least 90% identity to SEQ ID NO: 30.

Embodiment 69. The immune cell of embodiment 68, wherein the first polynucleotide and the third polynucleotide are contained within the polynucleotide sequence according to SEQ ID NO: 30.

Embodiment 70. The immune cell of embodiment 49, wherein the first polynucleotide and the third polynucleotide are contained within a polynucleotide sequence with at least 90% identity to SEQ ID NO: 31.

Embodiment 71. The immune cell of embodiment 70, wherein the first polynucleotide and the third polynucleotide are contained within the polynucleotide sequence according to SEQ ID NO: 31.

Embodiment 72. The immune cell of embodiment 49, wherein the first polynucleotide and the third polynucleotide are contained within a polynucleotide sequence with at least 90% identity to SEQ ID NO: 32.

Embodiment 73. The immune cell of embodiment 72, wherein the first polynucleotide and the third polynucleotide are contained within the polynucleotide sequence according to SEQ ID NO: 32.

Embodiment 74. The immune cell of embodiment 49, wherein the first polynucleotide and the third polynucleotide are contained within a polynucleotide sequence with at least 90% identity to SEQ ID NO: 33.

Embodiment 75. The immune cell of embodiment 74, wherein the first polynucleotide and the third polynucleotide are contained within the polynucleotide sequence according to SEQ ID NO: 33.

Embodiment 76. The immune cell of any one of embodiments 1-75, wherein the CAR amino acid sequence comprises a targeting moiety.

Embodiment 77. The immune cell of embodiment 76, wherein the targeting moiety binds to MUC1*.

Embodiment 78. The immune cell of embodiment 77, wherein the targeting moiety bind to the amino acid sequence according to SEQ ID NO: 35.

Embodiment 79. The immune cell of embodiment 77, wherein the targeting moiety bind to the amino acid sequence according to SEQ ID NO: 36.

Embodiment 80. The immune cell of any one of embodiments 76-79, wherein the targeting moiety comprises a heavy chain variable domain comprising heavy chain complementarity determining regions (CDR)s HC-CDR1, HC-CDR2, and HC-CDR3 and the targeting moiety comprises a light chain variable domain comprising light chain CDRs LC-CDR1, LC-CDR2, and LC-CDR3.

Embodiment 81. The immune cell of embodiment 80, wherein the HC-CDR1, HC-CDR2, and HC-CDR3 and the LC-CDR1, LC-CDR2, and LC-CDR3 comprise the amino acid sequences according to: HC-CDR1: SEQ ID NO: 37; HC-CDR2: SEQ ID NO: 38; HC-CDR3: SEQ ID NO: 39; LC-CDR1: SEQ ID NO: 40; LC-CDR2: SEQ ID NO: 41; and LC-CDR3: SEQ ID NO: 42.

Embodiment 82. The immune cell of embodiment 81, wherein the heavy chain variable domain comprises an amino acid sequence with at least 90% sequence identity to SEQ ID NO: 43 and the light chain variable domain comprises an amino acid sequence with at least 90% sequence identity to SEQ ID NO: 44.

Embodiment 83. The immune cell of any one of embodiments 81-82, wherein the heavy chain variable domain comprises the amino acid sequence of SEQ ID NO: 43 and the light chain variable domain comprises the amino acid sequence of SEQ ID NO: 44.

Embodiment 84. The immune cell of any one of embodiments 81-83, wherein the targeting moiety comprises a single chain variable fragment (scFv).

Embodiment 85. The immune cell of any one of embodiments 81-84, wherein the scFv comprises an amino acid sequence with at least 90% sequence identity to SEQ ID NO: 45.

Embodiment 86. The immune cell of any one of embodiments 81-85, wherein the scFv comprises the amino acid sequence of SEQ ID NO: 45.

Embodiment 87. The immune cell of embodiment 80, wherein the HC-CDR1, HC-CDR2, and HC-CDR3 and the LC-CDR1, LC-CDR2, and LC-CDR3 comprise the amino acid sequences according to: HC-CDR1: SEQ ID NO: 46; HC-CDR2: SEQ ID NO: 47; HC-CDR3: SEQ ID NO: 48; LC-CDR1: SEQ ID NO: 49; LC-CDR2: SEQ ID NO: 50; and LC-CDR3: SEQ ID NO: 51.

Embodiment 88. The immune cell of embodiment 87, wherein the heavy chain variable domain comprises an amino acid sequence with at least 90% sequence identity to SEQ ID NO: 52 and the light chain variable domain comprises an amino acid sequence with at least 90% sequence identity to SEQ ID NO: 53.

Embodiment 89. The immune cell of any one of embodiments 87-88, wherein the heavy chain variable domain comprises the amino acid sequence of SEQ ID NO: 52 and the light chain variable domain comprises the amino acid sequence of SEQ ID NO: 53.

Embodiment 90. The immune cell of any one of embodiments 87-89, wherein the targeting moiety comprises a single chain variable fragment (scFv).

Embodiment 91. The immune cell of any one of embodiments 87-90, wherein the scFv comprises an amino acid sequence with at least 90% sequence identity to SEQ ID NO: 54.

Embodiment 92. The immune cell of any one of embodiments 87-91, wherein the scFv comprises the amino acid sequence of SEQ ID NO: 54.

Embodiment 93. The immune cell of embodiment 80, wherein the HC-CDR1, HC-CDR2, and HC-CDR3 and the LC-CDR1, LC-CDR2, and LC-CDR3 comprise the amino acid sequences according to: HC-CDR1: SEQ ID NO: 55; HC-CDR2: SEQ ID NO: 56; HC-CDR3: SEQ ID NO: 57; LC-CDR1: SEQ ID NO: 58; LC-CDR2: SEQ ID NO: 59; and LC-CDR3: SEQ ID NO: 60.

Embodiment 94. The immune cell of embodiment 94, wherein the heavy chain variable domain comprises an amino acid sequence with at least 90% sequence identity to SEQ ID NO: 61 and the light chain variable domain comprises an amino acid sequence with at least 90% sequence identity to SEQ ID NO: 62.

Embodiment 95. The immune cell of any one of embodiments 93-94, wherein the heavy chain variable domain comprises the amino acid sequence of SEQ ID NO: 61 and the light chain variable domain comprises the amino acid sequence of SEQ ID NO: 62.

Embodiment 96. The immune cell of any one of embodiments 93-95, wherein the targeting moiety comprises a single chain variable fragment (scFv).

Embodiment 97. The immune cell of any one of embodiments 93-96, wherein the scFv comprises an amino acid sequence with at least 90% sequence identity to SEQ ID NO: 63.

Embodiment 98. The immune cell of any one of embodiments 93-97, wherein the scFv comprises the amino acid sequence of SEQ ID NO: 63.

Embodiment 99. The immune cell of any one of embodiments 1-98, wherein the CAR amino acid sequence comprises a hinge amino acid sequence.

Embodiment 100. The immune cell of embodiment 99, wherein the hinge amino acid sequence comprises a CD8 amino acid sequence.

Embodiment 101. The immune cell of embodiment 100, wherein the hinge amino acid sequence comprises an amino acid sequence with at least 90% sequence identity to SEQ ID NO: 64.

Embodiment 102. The immune cell of embodiment 100, wherein the hinge amino acid sequence comprises the amino acid sequence of SEQ ID NO: 64.

Embodiment 103. The immune cell of embodiment 99, wherein the hinge amino acid sequence comprises a CD28 amino acid sequence.

Embodiment 104. The immune cell of embodiment 103, wherein the hinge amino acid sequence comprises an amino acid sequence with at least 90% sequence identity to SEQ ID NO: 65.

Embodiment 105. The immune cell of embodiment 103, wherein the hinge amino acid sequence comprises the amino acid sequence of SEQ ID NO: 65.

Embodiment 106. The immune cell of any one of embodiments 1-98, wherein the CAR amino acid sequence comprises a transmembrane amino acid sequence.

Embodiment 107. The immune cell of embodiment 106, wherein the transmembrane amino acid sequence comprises a CD8 amino acid sequence.

Embodiment 108. The immune cell of embodiment 107, wherein the transmembrane amino acid sequence comprises an amino acid sequence with at least 90% sequence identity to SEQ ID NO: 66.

Embodiment 109. The immune cell of embodiment 107, wherein the transmembrane amino acid sequence comprises the amino acid sequence of SEQ ID NO: 66.

Embodiment 110. The immune cell of embodiment 106, wherein the transmembrane amino acid sequence comprises a CD28 amino acid sequence.

Embodiment 111. The immune cell of embodiment 110, wherein the transmembrane amino acid sequence comprises an amino acid sequence with at least 90% sequence identity to SEQ ID NO: 67.

Embodiment 112. The immune cell of embodiment 110, wherein the transmembrane amino acid sequence comprises the amino acid sequence of SEQ ID NO: 67.

Embodiment 113. The immune cell of any one of embodiments 1-98, wherein the CAR amino acid sequence comprises a co-stimulatory amino acid sequence.

Embodiment 114. The immune cell of embodiment 113, wherein the co-stimulatory amino acid sequence comprises a 41-BB amino acid sequence.

Embodiment 115. The immune cell of embodiment 114, wherein the co-stimulatory amino acid sequence comprises an amino acid sequence with at least 90% sequence identity to SEQ ID NO: 68.

Embodiment 116. The immune cell of embodiment 114, wherein the co-stimulatory amino acid sequence comprises the amino acid sequence of SEQ ID NO: 68.

Embodiment 117. The immune cell of embodiment 113, wherein the co-stimulatory amino acid sequence comprises a CD28 amino acid sequence.

Embodiment 118. The immune cell of embodiment 117, wherein the co-stimulatory amino acid sequence comprises an amino acid sequence with at least 90% sequence identity to SEQ ID NO: 69.

Embodiment 119. The immune cell of embodiment 117, wherein the co-stimulatory amino acid sequence comprises the amino acid sequence of SEQ ID NO: 69.

Embodiment 120. The immune cell of any one of embodiments 1-119, wherein the CAR amino acid sequence comprises a signaling amino acid sequence.

Embodiment 121. The immune cell of 120, wherein the signaling amino acid sequence comprises a CD3 amino acid sequence.

Embodiment 122. The immune cell of any one of embodiments 120-121, wherein the signaling amino acid sequence comprises an amino acid sequence with at least 90% sequence identity to SEQ ID NO: 70.

Embodiment 123. The immune cell of any one of embodiments 120-122, wherein the signaling amino acid sequence comprises the amino acid sequence of SEQ ID NO: 70.

Embodiment 124. The immune cell of 121, wherein the signaling amino acid sequence comprises a cytoplasmic region comprising a CD3ζ intracellular signaling domain comprising a mutation in an immunoreceptor tyrosine-based activation motif (ITAM).

Embodiment 125. The immune cell of 124, wherein the mutation in the ITAM comprises a point mutation at a tyrosine residue.

Embodiment 126. The immune cell of 125, wherein the tyrosine residue is mutated to a phenylalanine residue.

Embodiment 127. The immune cell of 124, wherein the mutation in the ITAM comprises a mutation to more than one ITAM of the CD3ζ intracellular signaling domain.

Embodiment 128. The immune cell of 127, wherein the mutation to more than one ITAM of the CD3ζ intracellular signaling domain comprises mutations to two ITAMs.

Embodiment 129. The immune cell of 128, wherein the two ITAMs comprises the second and third ITAM of the CD3ζ intracellular signaling domain.

Embodiment 130. The immune cell of 129, wherein the mutation in the ITAM comprises a single point mutation in at least two ITAMs of the CD3ζ intracellular signaling domain.

Embodiment 131. The immune cell of 124, wherein the mutation in the ITAM comprises two point mutations at two tyrosine residues wherein the two tyrosine residues are mutated to phenylalanine residues.

Embodiment 132. The immune cell of any one of embodiments 124-131, wherein the signaling amino acid sequence comprises an amino acid sequence with at least 90% sequence identity to SEQ ID NO: 71.

Embodiment 133. The immune cell of any one of embodiments 124-132, wherein the signaling amino acid sequence comprises the amino acid sequence of SEQ ID NO: 71.

Embodiment 134. The immune cell of any one of embodiments 1-133, wherein the CAR amino acid sequence comprises a hinge amino acid sequence according to SEQ ID NO: 64, a transmembrane amino acid sequence according to SEQ ID NO: 66, a co-stimulatory amino acid sequence according to SEQ ID NO: 68, and a signaling amino acid sequence according to SEQ ID NO: 70.

Embodiment 135. The immune cell of any one of embodiments 1-133, wherein the CAR amino acid sequence comprises a hinge amino acid sequence according to SEQ ID NO: 65, a transmembrane amino acid sequence according to SEQ ID NO: 67, a co-stimulatory amino acid sequence according to SEQ ID NO: 69, and a signaling amino acid sequence according to SEQ ID NO:70.

Embodiment 136. The immune cell of any one of embodiments 1-133, wherein the CAR amino acid sequence comprises a hinge amino acid sequence according to SEQ ID NO: 64, a transmembrane amino acid sequence according to SEQ ID NO: 66, a co-stimulatory amino acid sequence according to SEQ ID NO: 68, and a signaling amino acid sequence according to SEQ ID NO: 71.

Embodiment 137. The immune cell of any one of embodiments 1-133, wherein the CAR amino acid sequence comprises a hinge amino acid sequence according to SEQ ID NO: 65, a transmembrane amino acid sequence according to SEQ ID NO: 67, a co-stimulatory amino acid sequence according to SEQ ID NO: 69, and a signaling amino acid sequence according to SEQ ID NO:71.

Embodiment 138. The immune cell of any one of embodiments 1-133, wherein the CAR amino acid sequence comprises an amino acid sequence with at least 90% sequence identity to any one of SEQ ID NO: 72-83.

Embodiment 139. The immune cell of any one of embodiments 1-133, wherein the CAR amino acid sequence comprises the amino acid sequence of any one of SEQ ID NO: 72-83.

Embodiment 140. The immune cell of any one of embodiments 1-133, wherein the second polynucleotide comprises the polynucleotide sequence with at least 90% identity to any one of SEQ ID NO: 84-95.

Embodiment 141. The immune cell of any one of embodiments 1-133, wherein the second polynucleotide comprises the polynucleotide sequence according to any one of SEQ ID NO: 84-95.

Embodiment 142. The immune cell of any one of embodiments 1-133, wherein the first polynucleotide and the second polynucleotide are on the same vector.

Embodiment 143. The immune cell of any one of embodiments 1-133, wherein the first polynucleotide and the second polynucleotide are on the same vector and the vector comprises a polynucleotide sequence of at least 90% identity to any one of SEQ ID NOs: 96-190.

Embodiment 144. The immune cell of any one of embodiments 1-143, wherein the first polynucleotide and the second polynucleotide are on the same vector and the vector comprises a polynucleotide sequence of any one of SEQ ID NOs: 96-190.

Embodiment 145. The immune cell of any one of embodiments 1-143, wherein the first polynucleotide and the second polynucleotide are on separate vectors.

Embodiment 146. The immune cell of any one of embodiments 1-145, wherein the immune cell is a T cell.

Embodiment 147. The immune cell of any one of embodiments 1-145, wherein the immune cell is autologous.

Embodiment 148. The immune cell of any one of embodiments 1-145, wherein the immune cell is allogeneic.

Embodiment 149. A vector comprising the first polynucleotide sequence of any one of embodiments 1-148.

Embodiment 150. A vector comprising the second polynucleotide sequence of any one of embodiments 1-148.

Embodiment 151. A vector comprising the first polynucleotide sequence and the second polynucleotide sequence of any one of embodiments 1-148.

Embodiment 152. A method of treating cancer in a subject in need thereof comprises administering the immune cell of any of the above embodiments.

Embodiment 153. The method of embodiment 152, wherein the cancer comprises a blood cancer.

Embodiment 154. The method of embodiment 152, wherein the cancer comprises a solid tumor.

Embodiment 155. The method of embodiment 152, wherein the cancer comprises breast cancer, lung cancer, prostate cancer, ovarian cancer, colorectal cancer, pancreatic cancer.

Embodiment 156. The method of embodiment 152, wherein the cancer is a classified as a stage 1 cancer.

Embodiment 157. The method of embodiment 152, wherein the cancer is a classified as a stage 2 cancer.

Embodiment 158. The method of embodiment 152, wherein the cancer is a classified as a stage 3 cancer.

Embodiment 159. The method of embodiment 152, wherein the cancer is characterized by a low antigen expressing cell.

Embodiment 160. The method of embodiment 152, wherein the cancer is a classified as a stage 4 cancer.

Embodiment 161. The method of embodiment 152, wherein the cancer is characterized by a high antigen expressing cell.

Embodiment 162. A method of increasing CAR T persistence in a subject in need thereof comprising administering the immune cell of any of the above embodiments.

Embodiment 163. A method of inhibiting cancer recurrence in a subject in need thereof comprising administering the immune cell of any of the above embodiments.

Embodiment 164. A composition comprising the second polynucleotide of any of the above embodiments, wherein the second polynucleotide further comprises a leader sequence.

Embodiment 165. The composition according to embodiment 164, wherein the leader sequence increases uptake of the second polynucleotide into an immune cell.

Embodiment 166. The composition according to embodiment 164, wherein the leader sequence increases uptake of the second polynucleotide into a nucleus of an immune cell.

Embodiment 167. The composition of embodiment 164, wherein the second polynucleotide encodes an amino acid sequence according to SEQ ID NO: 34.

Embodiment 168. The composition of embodiment 164, wherein the second polynucleotide is targeted to a T-cell receptor α constant (TRAC) locus.

Embodiment 169. The composition of embodiment 164, wherein the second polynucleotide is targeted to a T-cell receptor α constant (TRAC) locus using CRISPR gene editing or TALEN gene editing.

Embodiment 170. A pharmaceutical composition comprising a) the immune cell of any one of embodiments 1-148, and b) a pharmaceutically acceptable excipient.

TABLE 7
Amino acid and polynucleotide sequences

| | | SEQ ID |
|---|---|---|
| Description | Sequence | NO: |
| IL18 receptor | MNCRELPLTLWVLISVSTAES | 1 |
| alpha signal | | |
| peptide | | |
| IL-18 receptor | CTSRPHITVVEGEPFYLKHCSCSLAHEIETTTKSWYKSSGSQEHVEL | 2 |
| alpha | NPRSSSRIALHDCVLEFWPVELNDTGSYFFQMKNYTQKWKLNVIRR | |
| extracellular | NKHSCFTERQVTSKIVEVKKFFQITCENSYYQTLVNSTSLYKNCKKL | |
| domain | LLENNKNPTIKKNAEFEDQGYYSCVHFLHHNGKLFNITKTFNITIVE | |
| | DRSNIVPVLLGPKLNHVAVELGKNVRLNCSALLNEEDVIYWMFGE | |
| | ENGSDPNIHEEKEMRIMTPEGKWHASKVLRIENIGESNLNVLYNCT | |
| | VASTGGTDTKSFILVRKADMADIPGHVFTR | |
| transmembrane | GMIIAVLILVAVVCLVTVCVI | 3 |
| IL-18 receptor | | |
| alpha | | |
| intracellular IL- | YRVDLVLFYRHLTRRDETLTDGKTYDAFVSYLKECRPENGEEHTFA | 4 |
| 18 receptor alpha | VEILPRVLEKHFGYKLCIFERDVVPGGAVVDEIHSLIEKSRRLIIVLSK | |
| | SYMSNEVRYELESGLHEALVERKIKIILIEFTPVTDFTFLPQSLKLLKS | |
| | HRVLKWKADKSLSYNSRFWKNLLYLMPAKTVKPGRDEPEVLPVLS | |
| | ES | |
| IL-18 receptor | MNCRELPLTLWVLISVSTAESCTSRPHITVVEGEPFYLKHCSCSLAH | 5 |
| alpha (full | EIETTTKSWYKSSGSQEHVELNPRSSSRIALHDCVLEFWPVELNDTG | |
| length) | SYFFQMKNYTQKWKLNVIRRNKHSCFTERQVTSKIVEVKKFFQITC | |
| | ENSYYQTLVNSTSLYKNCKKLLLENNKNPTIKKNAEFEDQGYYSCV | |
| | HFLHHNGKLFNITKTFNITIVEDRSNIVPVLLGPKLNHVAVELGKNV | |
| | RLNCSALLNEEDVIYWMFGEENGSDPNIHEEKEMRIMTPEGKWHA | |
| | SKVLRIENIGESNLNVLYNCTVASTGGTDTKSFILVRKADMADIPGH | |
| | VFTRGMIIAVLILVAVVCLVTVCVIYRVDLVLFYRHLTRRDETLTDG | |
| | KTYDAFVSYLKECRPENGEEHTFAVEILPRVLEKHFGYKLCIFERDV | |
| | VPGGAVVDEIHSLIEKSRRLIIVLSKSYMSNEVRYELESGLHEALVE | |
| | RKIKIILIEFTPVTDFTFLPQSLKLLKSHRVLKWKADKSLSYNSRFWK | |
| | NLLYLMPAKTVKPGRDEPEVLPVLSES | |
| IL-18 receptor | atgaattgtagagaattacccttgaccctttgggtgcttatatctgtaagcactgcagaatcttgtacttcacgtc | 6 |
| alpha (full | cccacattactgtggttgaaggggaacctttctatctgaaacattgctcgtgttcacttgcacatgagattgaaa | |
| length) | caaccaccaaaagctggtacaaaagcagtggatcacaggaacatgtggagctgaacccaaggagttcca | |
| polynucleotide | gcagaattgctttgcatgattgtgttttggagttttggccagttgagttgaatgacacaggatcttactttttccaa | |
| sequence | atgaaaaattatactcagaaatggaaattaaatgtcatcagaagaaataaacacagctgtttcactgaaagac | |
| | aagtaactagtaaaattgtggaagttaaaaaattttttcagataacctgtgaaaacagttactatcaaacactgg | |
| | tcaacagcacatcattgtataagaactgtaaaaagctactactggagaacaataaaaacccaacgataaaga | |
| | agaacgccgagtttgaagatcaggggtattactcctgcgtgcatttccttcatcataatggaaaactatttaata | |
| | tcaccaaaaccttcaatataacaatagtggaagatcgcagtaatatagttccggttcttcttggaccaaagctt | |
| | aaccatgttgcagtggaattaggaaaaaacgtaaggctcaactgctctgctttgctgaatgaagaggatgta | |
| | atttattggatgttcggggaagaaaatggatcggatcctaatatacatgaagagaaagaaatgagaattatga | |
| | ctccagaaggcaaatggcatgcttcaaaagtattgagaattgaaaatattggtgaaagcaatctaaatgtttta | |
| | tataattgcactgtggccagcacgggaggcacagacaccaaaagcttcatcttggtgagaaaagcagacat | |
| | ggctgatatcccaggccacgtcttcacaagaggaatgatcatagctgttttgatcttggtggcagtagtgtgc | |
| | ctagtgactgtgtgtgtcatttatagagttgacttggttctattttatagacatttaacgagaagagatgaaacatt | |
| | aacagatggaaaaacatatgatgcttttgtgtcttacctaaaagaatgccgacctgaaaatggagaggagca | |
| | cacctttgctgtggagattttgcccagggtgttggagaaacattttgggtataagttatgcatatttgaaaggga | |
| | tgtagtgcctggaggagctgttgttgatgaaatccactcactgatagagaaaagccgaagactaatcattgtc | |
| | ctaagtaaaagttatatgtctaatgaggtcaggtatgaacttgaaagtggactccatgaagcattggtggaaa | |
| | gaaaaattaaaataatcttaattgaatttacacctgttactgacttcacattcttgccccaatcactaaagcttttg | |
| | aaatctcacagagttctgaagtggaaggccgataaatctctttcttataactcaaggttctggaagaaccttctt | |
| | tacttaatgcctgcaaaaacagtcaagccaggtagagacgaaccggaagtcttgcctgttctttccgagtct | |
| IL18 receptor | MLCLGWIFLWLVAGERIKG | 7 |
| beta signal | | |
| peptide | | |
| IL-18 receptor | FNISGCSTKKLLWTYSTRSEEEFVLFCDLPEPQKSHFCHRNRLSPKQ | 8 |
| beta extracellular | VPEHLPFMGSNDLSDVQWYQQPSNGDPLEDIRKSYPHIIQDKCTLH | |
| domain | FLTPGVNNSGSYICRPKMIKSPYDVACCVKMILEVKPQTNASCEYS | |
| | ASHKQDLLLGSTGSISCPSLSCQSDAQSPAVTWYKNGKLLSVERSN | |
| | RIVVDEVYDYHQGTYVCDYTQSDTVSSWTVRAVVQVRTIVGDTKL | |
| | KPDILDPVEDTLEVELGKPLTISCKARFGFERVFNPVIKWYIKDSDLE | |
| | WEVSVPEAKSIKSTLKDEIIERNIILEKVTQRDLRRKFVCFVQNSIGN | |
| | TTQSVQLKEKR | |
| transmembrane | GVVLLYILLGTIGTLVAVLAA | 9 |
| IL-18 receptor | | |
| beta | | |
| intracellular IL- | SALLYRHWIEIVLLYRTYQSKDQTLGDKKDFDAFVSYAKWSSFPSE | 10 |
| 18 receptor beta | ATSSLSEEHLALSLFPDVLENKYGYSLCLLERDVAPGGVYAEDIVSII | |
| | KRSRRGIFILSPNYVNGPSIFELQAAVNLALDDQTLKLILIKFCYFQE | |
| | PESLPHLVKKALRVLPTVTWRGLKSVPPNSRFWAKMRYHMPVKNS | |
| | QGFTWNQLRITSRIFQWKGLSRTETTGRSSQPKEW | |
| IL-18 receptor | MLCLGWIFLWLVAGERIKGFNISGCSTKKLLWTYSTRSEEEFVLFC | 11 |
| beta (full length) | DLPEPQKSHFCHRNRLSPKQVPEHLPFMGSNDLSDVQWYQQPSNG | |
| | DPLEDIRKSYPHIIQDKCTLHFLTPGVNNSGSYICRPKMIKSPYDVAC | |
| | CVKMILEVKPQTNASCEYSASHKQDLLLGSTGSISCPSLSCQSDAQS | |
| | PAVTWYKNGKLLSVERSNRIVVDEVYDYHQGTYVCDYTQSDTVSS | |
| | WTVRAVVQVRTIVGDTKLKPDILDPVEDTLEVELGKPLTISCKARF | |
| | GFERVFNPVIKWYIKDSDLEWEVSVPEAKSIKSTLKDEIIERNIILEK | |
| | VTQRDLRRKFVCFVQNSIGNTTQSVQLKEKRGVVLLYILLGTIGTLV | |
| | AVLAASALLYRHWIEIVLLYRTYQSKDQTLGDKKDFDAFVSYAKW | |
| | SSFPSEATSSLSEEHLALSLFPDVLENKYGYSLCLLERDVAPGGVYA | |
| | EDIVSIIKRSRRGIFILSPNYVNGPSIFELQAAVNLALDDQTLKLILIKF | |
| | CYFQEPESLPHLVKKALRVLPTVTWRGLKSVPPNSRFWAKMRYHM | |
| | PVKNSQGFTWNQLRITSRIFQWKGLSRTETTGRSSQPKEW | |
| IL-18 receptor | atgctctgtttgggctggatatttctttggcttgttgcaggagagcgaattaaaggatttaatatttcaggttgttc | 12 |
| beta (full length) | cacaaaaaaactcctttggacatattctacaaggagtgaagaggaatttgtcttattttgtgatttaccagagcc | |
| polynucleotide | acagaaatcacatttctgccacagaaatcgactctcaccaaaacaagtccctgagcacctgcccttcatggg | |
| sequence | tagtaacgacctatctgatgtccaatggtaccaacaaccttcgaatggagatccattagaggacattaggaaa | |
| | agctatcctcacatcattcaggacaaatgtacccttcactttttgaccccaggggtgaataattctgggtcatat | |
| | atttgtagacccaagatgattaagagcccctatgatgtagcctgttgtgtcaagatgattttagaagttaagccc | |
| | cagacaaatgcatcctgtgagtattccgcatcacataagcaagacctacttcttgggagcactggctctatttc | |
| | ttgccccagtctcagctgccaaagtgatgcacaaagtccagcggtaacctggtacaagaatggaaaactcc | |
| | tctctgtggaaaggagcaaccgaatcgtagtggatgaagtttatgactatcaccagggcacatatgtatgtga | |
| | ttacactcagtcggatactgtgagttcgtggacagtcagagctgttgttcaagtgagaaccattgtgggagac | |
| | actaaactcaaaccagatattctggatcctgtcgaggacacactggaagtagaacttggaaagcctttaacta | |
| | ttagctgcaaagcacgatttggctttgaaagggtctttaaccctgtcataaaatggtacatcaaagattctgac | |
| | ctagagtgggaagtctcagtacctgaggcgaaaagtattaaatccactttaaaggatgaaatcattgagcgta | |
| | atatcatcttggaaaaagtcactcagcgtgatcttcgcaggaagtttgtttgctttgtccagaactccattggaa | |
| | acacaacccagtccgtccaactgaaagaaaagagaggagtggtgctcctgtacatcctgcttggcaccatc | |
| | gggaccctggtggccgtgctggcggcgagtgccctcctctacaggcactggattgaaatagtgctgctgta | |
| | ccggacctaccagagcaaggatcagacgcttggggataaaaaggattttgatgctttcgtatcctatgcaaa | |
| | atggagctcttttccaagtgaggccacttcatctctgagtgaagaacacttggccctgagcctatttcctgatgt | |
| | tttagaaaacaaatatggatatagcctgtgtttgcttgaaagagatgtggctccaggaggagtgtatgcagaa | |
| | gacattgtgagcattattaagagaagcagaagaggaatatttatcttgagccccaactatgtcaatggaccca | |
| | gtatctttgaactacaagcagcagtgaatcttgccttggatgatcaaacactgaaactcattttaattaagttctg | |
| | ttacttccaagagccagagtctctacctcatctcgtgaaaaaagctctcagggttttgcccacagttacttgga | |
| | gaggcttaaaatcagttcctcccaattctaggttctgggccaaaatgcgctaccacatgcctgtgaaaaactc | |
| | tcagggattcacgtggaaccagctcagaattacctctaggatttttcagtggaaaggactcagtagaacaga | |
| | aaccactgggaggagctcccagcctaaggaatgg | |
| FOXP3 NFAT | ggcttcattttttccatttactgcaga | 13 |
| response element | | |
| 3XFOXP3NFAT | ggcttcattttttccatttactgcagaggcttcattttttccatttactgcagaggcttcattttttccatttactgcag | 14 |
| (repeat of 3 | a | |
| FOXP3 NFAT | | |
| response | | |
| element) | | |
| 6XFOXP3NFAT | ggcttcattttttccatttactgcagaggcttcattttttccatttactgcagaggcttcattttttccatttactgcag | 15 |
| (repeat of 6 | aggcttcattttttccatttactgcagaggcttcattttttccatttactgcagaggcttcattttttccatttactgca | |
| FOXP3 NFAT | ga | |
| response | | |
| element) | | |
| Minimum CMV | taggcgtgtacggtgggaggcctatataagcagagctcgtttagtgaaccgtcagatcgcctggagacgcc | 16 |
| promoter | atccacgctgttttgacctccatagaagacaccgggaccgatccagc | |
| (mCMV) | | |
| 3XFOXP3NFAT | ggcttcattttttccatttactgcagaggcttcattttttccatttactgcagaggcttcattttttccatttactgcag | 17 |
| mCMV | aactagttaggcgtgtacggtgggaggcctatataagcagagctcgtttagtgaaccgtcagatcgcctgga | |
| | gacgccatccacgctgttttgacctccatagaagacaccgggaccgatccagc | |
| 6XFOXP3NFAT | ggcttcattttttccatttactgcagaggcttcattttttccatttactgcagaggcttcattttttccatttactgcag | 18 |
| | aggcttcattttttccatttactgcagaggcttcattttttccatttactgcagaggcttcattttttccatttactgca | |
| | gaactagttaggcgtgtacggtgggaggcctatataagcagagctcgtttagtgaaccgtcagatcgcctg | |
| | gagacgccatccacgctgttttgacctccatagaagacaccgggaccgatccagc | |
| IL2 NFAT | ggaggaaaaactgtttcatacagaaggcgt | 19 |
| response element | | |
| 3XIL2NFAT | ggaggaaaaactgtttcatacagaaggcgtggaggaaaaactgtttcatacagaaggcgtggaggaaaaa | 20 |
| (repeat of 3 IL2 | ctgtttcatacagaaggcgt | |
| NFAT response | | |
| element) | | |
| 6XIL2NFAT | ggaggaaaaactgtttcatacagaaggcgtggaggaaaaactgtttcatacagaaggcgtggaggaaaaa | 21 |
| (repeat of 6 IL2 | ctgtttcatacagaaggcgtggaggaaaaactgtttcatacagaaggcgtggaggaaaaactgtttcataca | |
| NFAT response | gaaggcgtggaggaaaaactgtttcatacagaaggcgtactagt | |
| element) | | |
| 3XIL2NFAT | ggaggaaaaactgtttcatacagaaggcgtggaggaaaaactgtttcatacagaaggcgtggaggaaaaa | 22 |
| mCMV | ctgtttcatacagaaggcgtactagttaggcgtgtacggtgggaggcctatataagcagagctcgtttagtga | |
| | accgtcagatcgcctggagacgccatccacgctgttttgacctccatagaagacaccgggaccgatccag | |
| | c | |
| 6XIL2NFAT | ggaggaaaaactgtttcatacagaaggcgtggaggaaaaactgtttcatacagaaggcgtggaggaaaaa | 23 |
| | ctgtttcatacagaaggcgtggaggaaaaactgtttcatacagaaggcgtggaggaaaaactgtttcataca | |
| | gaaggcgtggaggaaaaactgtttcatacagaaggcgtactagttaggcgtgtacggtgggaggcctatat | |
| | aagcagagctcgtttagtgaaccgtcagatcgcctggagacgccatccacgctgttttgacctccatagaag | |
| | acaccgggaccgatccagc | |
| T2A self- | ggaagcggtgagggaagaggtagcctgctcacgtgcggtgatgtggaggagaaccctgggcct | 24 |
| cleaving peptide, | | |
| nucleotide | | |
| sequence | | |
| T2A self- | GSGEGRGSLLTCGDVEENPGP | 25 |
| cleaving peptide, | | |
| amino acid | | |
| sequence | | |
| NFAT inducible | ggcttcattttttccatttactgcagaggcttcattttttccatttactgcagaggcttcattttttccatttactgcag | 26 |
| IL-18 receptor | aggcttcattttttccatttactgcagaggcttcattttttccatttactgcagaggcttcattttttccatttactgca | |
| alpha, | gaactagttaggcgtgtacggtgggaggcctatataagcagagctcgtttagtgaaccgtcagatcgcctg | |
| 6XFOXP3NFAT | gagacgccatccacgctgttttgacctccatagaagacaccgggaccgatccagcctcgagagacccaat | |
| mCMV IL18 | gctagccaccatgaattgtagagaattacccttgaccctttgggtgcttatatctgtaagcactgcagaatctt | |
| receptor alpha | gtacttcacgtccccacattactgtggttgaaggggaacctttctatctgaaacattgctcgtgttcacttgcac | |
| | atgagattgaaacaaccaccaaaagctggtacaaaagcagtggatcacaggaacatgtggagctgaaccc | |
| | aaggagttccagcagaattgctttgcatgattgtgttttggagttttggccagttgagttgaatgacacaggatc | |
| | ttactttttccaaatgaaaaattatactcagaaatggaaattaaatgtcatcagaagaaataaacacagctgtttc | |
| | actgaaagacaagtaactagtaaaattgtggaagttaaaaaattttttcagataacctgtgaaaacagttactat | |
| | caaacactggtcaacagcacatcattgtataagaactgtaaaaagctactactggagaacaataaaaaccca | |
| | acgataaagaagaacgccgagtttgaagatcaggggtattactcctgcgtgcatttccttcatcataatggaa | |
| | aactatttaatatcaccaaaaccttcaatataacaatagtggaagatcgcagtaatatagttccggttcttcttgg | |
| | accaaagcttaaccatgttgcagtggaattaggaaaaaacgtaaggctcaactgctctgctttgctgaatgaa | |
| | gaggatgtaatttattggatgttcggggaagaaaatggatcggatcctaatatacatgaagagaaagaaatg | |
| | agaattatgactccagaaggcaaatggcatgcttcaaaagtattgagaattgaaaatattggtgaaagcaatc | |
| | taaatgttttatataattgcactgtggccagcacgggaggcacagacaccaaaagcttcatcttggtgagaaa | |
| | agcagacatggctgatatcccaggccacgtcttcacaagaggaatgatcatagctgttttgatcttggtggca | |
| | gtagtgtgcctagtgactgtgtgtgtcatttatagagttgacttggttctattttatagacatttaacgagaagag | |
| | atgaaacattaacagatggaaaaacatatgatgcttttgtgtcttacctaaaagaatgccgacctgaaaatgg | |
| | agaggagcacacctttgctgtggagattttgcccagggtgttggagaaacattttgggtataagttatgcatat | |
| | ttgaaagggatgtagtgcctggaggagctgttgttgatgaaatccactcactgatagagaaaagccgaaga | |
| | ctaatcattgtcctaagtaaaagttatatgtctaatgaggtcaggtatgaacttgaaagtggactccatgaagc | |
| | attggtggaaagaaaaattaaaataatcttaattgaatttacacctgttactgacttcacattcttgccccaatca | |
| | ctaaagcttttgaaatctcacagagttctgaagtggaaggccgataaatctctttcttataactcaaggttctgg | |
| | aagaaccttctttacttaatgcctgcaaaaacagtcaagccaggtagagacgaaccggaagtcttgcctgttc | |
| | tttccgagtcttgataa | |
| NFAT inducible | ggcttcattttttccatttactgcagaggcttcattttttccatttactgcagaggcttcattttttccatttactgcag | 27 |
| IL-18 receptor | aactagttaggcgtgtacggtgggaggcctatataagcagagctcgtttagtgaaccgtcagatcgcctgga | |
| alpha, | gacgccatccacgctgttttgacctccatagaagacaccgggaccgatccagcctcgagagacccaatgct | |
| 3XFOXP3NFAT | agccaccatgaattgtagagaattacccttgaccctttgggtgcttatatctgtaagcactgcagaatcttgta | |
| mCMV IL18 | cttcacgtccccacattactgtggttgaaggggaacctttctatctgaaacattgctcgtgttcacttgcacatg | |
| receptor alpha | agattgaaacaaccaccaaaagctggtacaaaagcagtggatcacaggaacatgtggagctgaacccaa | |
| | ggagttccagcagaattgctttgcatgattgtgttttggagttttggccagttgagttgaatgacacaggatctt | |
| | actttttccaaatgaaaaattatactcagaaatggaaattaaatgtcatcagaagaaataaacacagctgtttca | |
| | ctgaaagacaagtaactagtaaaattgtggaagttaaaaaattttttcagataacctgtgaaaacagttactatc | |
| | aaacactggtcaacagcacatcattgtataagaactgtaaaaagctactactggagaacaataaaaacccaa | |
| | cgataaagaagaacgccgagtttgaagatcaggggtattactcctgcgtgcatttccttcatcataatggaaa | |
| | actatttaatatcaccaaaaccttcaatataacaatagtggaagatcgcagtaatatagttccggttcttcttgga | |
| | ccaaagcttaaccatgttgcagtggaattaggaaaaaacgtaaggctcaactgctctgctttgctgaatgaag | |
| | aggatgtaatttattggatgttcggggaagaaaatggatcggatcctaatatacatgaagagaaagaaatga | |
| | gaattatgactccagaaggcaaatggcatgcttcaaaagtattgagaattgaaaatattggtgaaagcaatct | |
| | aaatgttttatataattgcactgtggccagcacgggaggcacagacaccaaaagcttcatcttggtgagaaa | |
| | agcagacatggctgatatcccaggccacgtcttcacaagaggaatgatcatagctgttttgatcttggtggca | |
| | gtagtgtgcctagtgactgtgtgtgtcatttatagagttgacttggttctattttatagacatttaacgagaagag | |
| | atgaaacattaacagatggaaaaacatatgatgcttttgtgtcttacctaaaagaatgccgacctgaaaatgg | |
| | agaggagcacacctttgctgtggagattttgcccagggtgttggagaaacattttgggtataagttatgcatat | |
| | ttgaaagggatgtagtgcctggaggagctgttgttgatgaaatccactcactgatagagaaaagccgaaga | |
| | ctaatcattgtcctaagtaaaagttatatgtctaatgaggtcaggtatgaacttgaaagtggactccatgaagc | |
| | attggtggaaagaaaaattaaaataatcttaattgaatttacacctgttactgacttcacattcttgccccaatca | |
| | ctaaagcttttgaaatctcacagagttctgaagtggaaggccgataaatctctttcttataactcaaggttctgg | |
| | aagaaccttctttacttaatgcctgcaaaaacagtcaagccaggtagagacgaaccggaagtcttgcctgttc | |
| | tttccgagtcttgataa | |
| NFAT inducible | ggaggaaaaactgtttcatacagaaggcgtggaggaaaaactgtttcatacagaaggcgtggaggaaaaa | 28 |
| IL-18 receptor | ctgtttcatacagaaggcgtggaggaaaaactgtttcatacagaaggcgtggaggaaaaactgtttcataca | |
| alpha, | gaaggcgtggaggaaaaactgtttcatacagaaggcgtactagttaggcgtgtacggtgggaggcctatat | |
| 6XIL2NFAT | aagcagagctcgtttagtgaaccgtcagatcgcctggagacgccatccacgctgttttgacctccatagaag | |
| mCMV IL18 | acaccgggaccgatccagcctcgagagacccaatgctagccaccatgaattgtagagaattacccttgac | |
| receptor alpha | cctttgggtgcttatatctgtaagcactgcagaatcttgtacttcacgtccccacattactgtggttgaagggga | |
| | acctttctatctgaaacattgctcgtgttcacttgcacatgagattgaaacaaccaccaaaagctggtacaaaa | |
| | gcagtggatcacaggaacatgtggagctgaacccaaggagttccagcagaattgctttgcatgattgtgtttt | |
| | ggagttttggccagttgagttgaatgacacaggatcttactttttccaaatgaaaaattatactcagaaatggaa | |
| | attaaatgtcatcagaagaaataaacacagctgtttcactgaaagacaagtaactagtaaaattgtggaagtta | |
| | aaaaattttttcagataacctgtgaaaacagttactatcaaacactggtcaacagcacatcattgtataagaact | |
| | gtaaaaagctactactggagaacaataaaaacccaacgataaagaagaacgccgagtttgaagatcaggg | |
| | gtattactcctgcgtgcatttccttcatcataatggaaaactatttaatatcaccaaaaccttcaatataacaatag | |
| | tggaagatcgcagtaatatagttccggttcttcttggaccaaagcttaaccatgttgcagtggaattaggaaaa | |
| | aacgtaaggctcaactgctctgctttgctgaatgaagaggatgtaatttattggatgttcggggaagaaaatg | |
| | gatcggatcctaatatacatgaagagaaagaaatgagaattatgactccagaaggcaaatggcatgcttcaa | |
| | aagtattgagaattgaaaatattggtgaaagcaatctaaatgttttatataattgcactgtggccagcacggga | |
| | ggcacagacaccaaaagcttcatcttggtgagaaaagcagacatggctgatatcccaggccacgtcttcac | |
| | aagaggaatgatcatagctgttttgatcttggtggcagtagtgtgcctagtgactgtgtgtgtcatttatagagtt | |
| | gacttggttctattttatagacatttaacgagaagagatgaaacattaacagatggaaaaacatatgatgctttt | |
| | gtgtcttacctaaaagaatgccgacctgaaaatggagaggagcacacctttgctgtggagattttgcccagg | |
| | gtgttggagaaacattttgggtataagttatgcatatttgaaagggatgtagtgcctggaggagctgttgttgat | |
| | gaaatccactcactgatagagaaaagccgaagactaatcattgtcctaagtaaaagttatatgtctaatgagg | |
| | tcaggtatgaacttgaaagtggactccatgaagcattggtggaaagaaaaattaaaataatcttaattgaattt | |
| | acacctgttactgacttcacattcttgccccaatcactaaagcttttgaaatctcacagagttctgaagtggaag | |
| | gccgataaatctctttcttataactcaaggttctggaagaaccttctttacttaatgcctgcaaaaacagtcaag | |
| | ccaggtagagacgaaccggaagtcttgcctgttctttccgagtcttgataa | |
| NF AT inducible | ggaggaaaaactgtttcatacagaaggcgtggaggaaaaactgtttcatacagaaggcgtggaggaaaaa | 29 |
| IL-18 receptor | ctgtttcatacagaaggcgtactagttaggcgtgtacggtgggaggcctatataagcagagctcgtttagtga | |
| alpha, | accgtcagatcgcctggagacgccatccacgctgttttgacctccatagaagacaccgggaccgatccag | |
| 3XIL2NFAT | cctcgagagacccaatgctagccaccatgaattgtagagaattacccttgaccctttgggtgcttatatctgt | |
| mCMV IL18 | aagcactgcagaatcttgtacttcacgtccccacattactgtggttgaaggggaacctttctatctgaaacatt | |
| receptor alpha | gctcgtgttcacttgcacatgagattgaaacaaccaccaaaagctggtacaaaagcagtggatcacaggaa | |
| | catgtggagctgaacccaaggagttccagcagaattgctttgcatgattgtgttttggagttttggccagttga | |
| | gttgaatgacacaggatcttactttttccaaatgaaaaattatactcagaaatggaaattaaatgtcatcagaag | |
| | aaataaacacagctgtttcactgaaagacaagtaactagtaaaattgtggaagttaaaaaattttttcagataac | |
| | ctgtgaaaacagttactatcaaacactggtcaacagcacatcattgtataagaactgtaaaaagctactactg | |
| | gagaacaataaaaacccaacgataaagaagaacgccgagtttgaagatcaggggtattactcctgcgtgc | |
| | atttccttcatcataatggaaaactatttaatatcaccaaaaccttcaatataacaatagtggaagatcgcagta | |
| | atatagttccggttcttcttggaccaaagcttaaccatgttgcagtggaattaggaaaaaacgtaaggctcaac | |
| | tgctctgctttgctgaatgaagaggatgtaatttattggatgttcggggaagaaaatggatcggatcctaatat | |
| | acatgaagagaaagaaatgagaattatgactccagaaggcaaatggcatgcttcaaaagtattgagaattga | |
| | aaatattggtgaaagcaatctaaatgttttatataattgcactgtggccagcacgggaggcacagacaccaa | |
| | aagcttcatcttggtgagaaaagcagacatggctgatatcccaggccacgtcttcacaagaggaatgatcat | |
| | agctgttttgatcttggtggcagtagtgtgcctagtgactgtgtgtgtcatttatagagttgacttggttctatttta | |
| | tagacatttaacgagaagagatgaaacattaacagatggaaaaacatatgatgcttttgtgtcttacctaaaag | |
| | aatgccgacctgaaaatggagaggagcacacctttgctgtggagattttgcccagggtgttggagaaacatt | |
| | ttgggtataagttatgcatatttgaaagggatgtagtgcctggaggagctgttgttgatgaaatccactcactg | |
| | atagagaaaagccgaagactaatcattgtcctaagtaaaagttatatgtctaatgaggtcaggtatgaacttga | |
| | aagtggactccatgaagcattggtggaaagaaaaattaaaataatcttaattgaatttacacctgttactgactt | |
| | cacattcttgccccaatcactaaagcttttgaaatctcacagagttctgaagtggaaggccgataaatctctttc | |
| | ttataactcaaggttctggaagaaccttctttacttaatgcctgcaaaaacagtcaagccaggtagagacgaa | |
| | ccggaagtcttgcctgttctttccgagtcttgataa | |
| NFAT inducible | ggaggaaaaactgtttcatacagaaggcgtggaggaaaaactgtttcatacagaaggcgtggaggaaaaa | 30 |
| IL-18 receptor | ctgtttcatacagaaggcgtggaggaaaaactgtttcatacagaaggcgtggaggaaaaactgtttcataca | |
| alpha and beta, | gaaggcgtggaggaaaaactgtttcatacagaaggcgtactagttaggcgtgtacggtgggaggcctatat | |
| 6XFOXP3NFAT | aagcagagctcgtttagtgaaccgtcagatcgcctggagacgccatccacgctgttttgacctccatagaag | |
| mCMV IL18 | acaccgggaccgatccagcctcgagagacccaatgctagccaccatgaattgtagagaattacccttgac | |
| receptor alpha- | cctttgggtgcttatatctgtaagcactgcagaatcttgtacttcacgtccccacattactgtggttgaagggga | |
| T2A-IL18Rb | acctttctatctgaaacattgctcgtgttcacttgcacatgagattgaaacaaccaccaaaagctggtacaaaa | |
| | gcagtggatcacaggaacatgtggagctgaacccaaggagttccagcagaattgctttgcatgattgtgtttt | |
| | ggagttttggccagttgagttgaatgacacaggatcttactttttccaaatgaaaaattatactcagaaatggaa | |
| | attaaatgtcatcagaagaaataaacacagctgtttcactgaaagacaagtaactagtaaaattgtggaagtta | |
| | aaaaattttttcagataacctgtgaaaacagttactatcaaacactggtcaacagcacatcattgtataagaact | |
| | gtaaaaagctactactggagaacaataaaaacccaacgataaagaagaacgccgagtttgaagatcaggg | |
| | gtattactcctgcgtgcatttccttcatcataatggaaaactatttaatatcaccaaaaccttcaatataacaatag | |
| | tggaagatcgcagtaatatagttccggttcttcttggaccaaagcttaaccatgttgcagtggaattaggaaaa | |
| | aacgtaaggctcaactgctctgctttgctgaatgaagaggatgtaatttattggatgttcggggaagaaaatg | |
| | gatcggatcctaatatacatgaagagaaagaaatgagaattatgactccagaaggcaaatggcatgcttcaa | |
| | aagtattgagaattgaaaatattggtgaaagcaatctaaatgttttatataattgcactgtggccagcacggga | |
| | ggcacagacaccaaaagcttcatcttggtgagaaaagcagacatggctgatatcccaggccacgtcttcac | |
| | aagaggaatgatcatagctgttttgatcttggtggcagtagtgtgcctagtgactgtgtgtgtcatttatagagtt | |
| | gacttggttctattttatagacatttaacgagaagagatgaaacattaacagatggaaaaacatatgatgctttt | |
| | gtgtcttacctaaaagaatgccgacctgaaaatggagaggagcacacctttgctgtggagattttgcccagg | |
| | gtgttggagaaacattttgggtataagttatgcatatttgaaagggatgtagtgcctggaggagctgttgttgat | |
| | gaaatccactcactgatagagaaaagccgaagactaatcattgtcctaagtaaaagttatatgtctaatgagg | |
| | tcaggtatgaacttgaaagtggactccatgaagcattggtggaaagaaaaattaaaataatcttaattgaattt | |
| | acacctgttactgacttcacattcttgccccaatcactaaagcttttgaaatctcacagagttctgaagtggaag | |
| | gccgataaatctctttcttataactcaaggttctggaagaaccttctttacttaatgcctgcaaaaacagtcaag | |
| | ccaggtagagacgaaccggaagtcttgcctgttctttccgagtctggaagcggtgagggaagaggtagcct | |
| | gctcacgtgcggtgatgtggaggagaaccctgggcctatgctctgtttgggctggatatttctttggcttgttg | |
| | caggagagcgaattaaaggatttaatatttcaggttgttccacaaaaaaactcctttggacatattctacaagg | |
| | agtgaagaggaatttgtcttattttgtgatttaccagagccacagaaatcacatttctgccacagaaatcgactc | |
| | tcaccaaaacaagtccctgagcacctgcccttcatgggtagtaacgacctatctgatgtccaatggtaccaa | |
| | caaccttcgaatggagatccattagaggacattaggaaaagctatcctcacatcattcaggacaaatgtacc | |
| | cttcactttttgaccccaggggtgaataattctgggtcatatatttgtagacccaagatgattaagagcccctat | |
| | gatgtagcctgttgtgtcaagatgattttagaagttaagccccagacaaatgcatcctgtgagtattccgcatc | |
| | acataagcaagacctacttcttgggagcactggctctatttcttgccccagtctcagctgccaaagtgatgca | |
| | caaagtccagcggtaacctggtacaagaatggaaaactcctctctgtggaaaggagcaaccgaatcgtagt | |
| | ggatgaagtttatgactatcaccagggcacatatgtatgtgattacactcagtcggatactgtgagttcgtgga | |
| | cagtcagagctgttgttcaagtgagaaccattgtgggagacactaaactcaaaccagatattctggatcctgt | |
| | cgaggacacactggaagtagaacttggaaagcctttaactattagctgcaaagcacgatttggctttgaaag | |
| | ggtctttaaccctgtcataaaatggtacatcaaagattctgacctagagtgggaagtctcagtacctgaggcg | |
| | aaaagtattaaatccactttaaaggatgaaatcattgagcgtaatatcatcttggaaaaagtcactcagcgtga | |
| | tcttcgcaggaagtttgtttgctttgtccagaactccattggaaacacaacccagtccgtccaactgaaagaa | |
| | aagagaggagtggtgctcctgtacatcctgcttggcaccatcgggaccctggtggccgtgctggcggcga | |
| | gtgccctcctctacaggcactggattgaaatagtgctgctgtaccggacctaccagagcaaggatcagacg | |
| | cttggggataaaaaggattttgatgctttcgtatcctatgcaaaatggagctcttttccaagtgaggccacttca | |
| | tctctgagtgaagaacacttggccctgagcctatttcctgatgttttagaaaacaaatatggatatagcctgtgt | |
| | ttgcttgaaagagatgtggctccaggaggagtgtatgcagaagacattgtgagcattattaagagaagcaga | |
| | agaggaatatttatcttgagccccaactatgtcaatggacccagtatctttgaactacaagcagcagtgaatct | |
| | tgccttggatgatcaaacactgaaactcattttaattaagttctgttacttccaagagccagagtctctacctcat | |
| | ctcgtgaaaaaagctctcagggttttgcccacagttacttggagaggcttaaaatcagttcctcccaattctag | |
| | gttctgggccaaaatgcgctaccacatgcctgtgaaaaactctcagggattcacgtggaaccagctcagaat | |
| | tacctctaggatttttcagtggaaaggactcagtagaacagaaaccactgggaggagctcccagcctaagg | |
| | aatggtgataa | |
| NFAT inducible | ggcttcattttttccatttactgcagaggcttcattttttccatttactgcagaggcttcattttttccatttactgcag | 31 |
| IL-18 receptor | aactagttaggcgtgtacggtgggaggcctatataagcagagctcgtttagtgaaccgtcagatcgcctgga | |
| alpha and beta, | gacgccatccacgctgttttgacctccatagaagacaccgggaccgatccagcctcgagagacccaatgct | |
| 3XFOXP3NFAT | agccaccatgaattgtagagaattacccttgaccctttgggtgcttatatctgtaagcactgcagaatcttgta | |
| mCMV IL18 | cttcacgtccccacattactgtggttgaaggggaacctttctatctgaaacattgctcgtgttcacttgcacatg | |
| receptor alpha- | agattgaaacaaccaccaaaagctggtacaaaagcagtggatcacaggaacatgtggagctgaacccaa | |
| T2A-IL18Rb | ggagttccagcagaattgctttgcatgattgtgttttggagttttggccagttgagttgaatgacacaggatctt | |
| | actttttccaaatgaaaaattatactcagaaatggaaattaaatgtcatcagaagaaataaacacagctgtttca | |
| | ctgaaagacaagtaactagtaaaattgtggaagttaaaaaattttttcagataacctgtgaaaacagttactatc | |
| | aaacactggtcaacagcacatcattgtataagaactgtaaaaagctactactggagaacaataaaaacccaa | |
| | cgataaagaagaacgccgagtttgaagatcaggggtattactcctgcgtgcatttccttcatcataatggaaa | |
| | actatttaatatcaccaaaaccttcaatataacaatagtggaagatcgcagtaatatagttccggttcttcttgga | |
| | ccaaagcttaaccatgttgcagtggaattaggaaaaaacgtaaggctcaactgctctgctttgctgaatgaag | |
| | aggatgtaatttattggatgttcggggaagaaaatggatcggatcctaatatacatgaagagaaagaaatga | |
| | gaattatgactccagaaggcaaatggcatgcttcaaaagtattgagaattgaaaatattggtgaaagcaatct | |
| | aaatgttttatataattgcactgtggccagcacgggaggcacagacaccaaaagcttcatcttggtgagaaa | |
| | agcagacatggctgatatcccaggccacgtcttcacaagaggaatgatcatagctgttttgatcttggtggca | |
| | gtagtgtgcctagtgactgtgtgtgtcatttatagagttgacttggttctattttatagacatttaacgagaagag | |
| | atgaaacattaacagatggaaaaacatatgatgcttttgtgtcttacctaaaagaatgccgacctgaaaatgg | |
| | agaggagcacacctttgctgtggagattttgcccagggtgttggagaaacattttgggtataagttatgcatat | |
| | ttgaaagggatgtagtgcctggaggagctgttgttgatgaaatccactcactgatagagaaaagccgaaga | |
| | ctaatcattgtcctaagtaaaagttatatgtctaatgaggtcaggtatgaacttgaaagtggactccatgaagc | |
| | attggtggaaagaaaaattaaaataatcttaattgaatttacacctgttactgacttcacattcttgccccaatca | |
| | ctaaagcttttgaaatctcacagagttctgaagtggaaggccgataaatctctttcttataactcaaggttctgg | |
| | aagaaccttctttacttaatgcctgcaaaaacagtcaagccaggtagagacgaaccggaagtcttgcctgttc | |
| | tttccgagtctggaagcggtgagggaagaggtagcctgctcacgtgcggtgatgtggaggagaaccctgg | |
| | gcctatgctctgtttgggctggatatttctttggcttgttgcaggagagcgaattaaaggatttaatatttcaggtt | |
| | gttccacaaaaaaactcctttggacatattctacaaggagtgaagaggaatttgtcttattttgtgatttaccaga | |
| | gccacagaaatcacatttctgccacagaaatcgactctcaccaaaacaagtccctgagcacctgcccttcat | |
| | gggtagtaacgacctatctgatgtccaatggtaccaacaaccttcgaatggagatccattagaggacattag | |
| | gaaaagctatcctcacatcattcaggacaaatgtacccttcactttttgaccccaggggtgaataattctgggt | |
| | catatatttgtagacccaagatgattaagagcccctatgatgtagcctgttgtgtcaagatgattttagaagtta | |
| | agccccagacaaatgcatcctgtgagtattccgcatcacataagcaagacctacttcttgggagcactggct | |
| | ctatttcttgccccagtctcagctgccaaagtgatgcacaaagtccagcggtaacctggtacaagaatggaa | |
| | aactcctctctgtggaaaggagcaaccgaatcgtagtggatgaagtttatgactatcaccagggcacatatgt | |
| | atgtgattacactcagtcggatactgtgagttcgtggacagtcagagctgttgttcaagtgagaaccattgtgg | |
| | gagacactaaactcaaaccagatattctggatcctgtcgaggacacactggaagtagaacttggaaagcctt | |
| | taactattagctgcaaagcacgatttggctttgaaagggtctttaaccctgtcataaaatggtacatcaaagatt | |
| | ctgacctagagtgggaagtctcagtacctgaggcgaaaagtattaaatccactttaaaggatgaaatcattga | |
| | gcgtaatatcatcttggaaaaagtcactcagcgtgatcttcgcaggaagtttgtttgctttgtccagaactccat | |
| | tggaaacacaacccagtccgtccaactgaaagaaaagagaggagtggtgctcctgtacatcctgcttggca | |
| | ccatcgggaccctggtggccgtgctggcggcgagtgccctcctctacaggcactggattgaaatagtgctg | |
| | ctgtaccggacctaccagagcaaggatcagacgcttggggataaaaaggattttgatgctttcgtatcctatg | |
| | caaaatggagctcttttccaagtgaggccacttcatctctgagtgaagaacacttggccctgagcctatttcct | |
| | gatgttttagaaaacaaatatggatatagcctgtgtttgcttgaaagagatgtggctccaggaggagtgtatgc | |
| | agaagacattgtgagcattattaagagaagcagaagaggaatatttatcttgagccccaactatgtcaatgga | |
| | cccagtatctttgaactacaagcagcagtgaatcttgccttggatgatcaaacactgaaactcattttaattaag | |
| | ttctgttacttccaagagccagagtctctacctcatctcgtgaaaaaagctctcagggttttgcccacagttact | |
| | tggagaggcttaaaatcagttcctcccaattctaggttctgggccaaaatgcgctaccacatgcctgtgaaaa | |
| | actctcagggattcacgtggaaccagctcagaattacctctaggatttttcagtggaaaggactcagtagaac | |
| | agaaaccactgggaggagctcccagcctaaggaatggtgataa | |
| NFAT inducible | ggaggaaaaactgtttcatacagaaggcgtggaggaaaaactgtttcatacagaaggcgtggaggaaaaa | 32 |
| IL-18 receptor | ctgtttcatacagaaggcgtggaggaaaaactgtttcatacagaaggcgtggaggaaaaactgtttcataca | |
| alpha and beta, | gaaggcgtggaggaaaaactgtttcatacagaaggcgtactagttaggcgtgtacggtgggaggcctatat | |
| 6XIL2NFAT | aagcagagctcgtttagtgaaccgtcagatcgcctggagacgccatccacgctgttttgacctccatagaag | |
| mCMV IL18 | acaccgggaccgatccagcctcgagagacccaatgctagccaccatgaattgtagagaattacccttgac | |
| receptor alpha- | cctttgggtgcttatatctgtaagcactgcagaatcttgtacttcacgtccccacattactgtggttgaagggga | |
| T2A-IL18Rb | acctttctatctgaaacattgctcgtgttcacttgcacatgagattgaaacaaccaccaaaagctggtacaaaa | |
| | gcagtggatcacaggaacatgtggagctgaacccaaggagttccagcagaattgctttgcatgattgtgtttt | |
| | ggagttttggccagttgagttgaatgacacaggatcttactttttccaaatgaaaaattatactcagaaatggaa | |
| | attaaatgtcatcagaagaaataaacacagctgtttcactgaaagacaagtaactagtaaaattgtggaagtta | |
| | aaaaattttttcagataacctgtgaaaacagttactatcaaacactggtcaacagcacatcattgtataagaact | |
| | gtaaaaagctactactggagaacaataaaaacccaacgataaagaagaacgccgagtttgaagatcaggg | |
| | gtattactcctgcgtgcatttccttcatcataatggaaaactatttaatatcaccaaaaccttcaatataacaatag | |
| | tggaagatcgcagtaatatagttccggttcttcttggaccaaagcttaaccatgttgcagtggaattaggaaaa | |
| | aacgtaaggctcaactgctctgctttgctgaatgaagaggatgtaatttattggatgttcggggaagaaaatg | |
| | gatcggatcctaatatacatgaagagaaagaaatgagaattatgactccagaaggcaaatggcatgcttcaa | |
| | aagtattgagaattgaaaatattggtgaaagcaatctaaatgttttatataattgcactgtggccagcacggga | |
| | ggcacagacaccaaaagcttcatcttggtgagaaaagcagacatggctgatatcccaggccacgtcttcac | |
| | aagaggaatgatcatagctgttttgatcttggtggcagtagtgtgcctagtgactgtgtgtgtcatttatagagtt | |
| | gacttggttctattttatagacatttaacgagaagagatgaaacattaacagatggaaaaacatatgatgctttt | |
| | gtgtcttacctaaaagaatgccgacctgaaaatggagaggagcacacctttgctgtggagattttgcccagg | |
| | gtgttggagaaacattttgggtataagttatgcatatttgaaagggatgtagtgcctggaggagctgttgttgat | |
| | gaaatccactcactgatagagaaaagccgaagactaatcattgtcctaagtaaaagttatatgtctaatgagg | |
| | tcaggtatgaacttgaaagtggactccatgaagcattggtggaaagaaaaattaaaataatcttaattgaattt | |
| | acacctgttactgacttcacattcttgccccaatcactaaagcttttgaaatctcacagagttctgaagtggaag | |
| | gccgataaatctctttcttataactcaaggttctggaagaaccttctttacttaatgcctgcaaaaacagtcaag | |
| | ccaggtagagacgaaccggaagtcttgcctgttctttccgagtctggaagcggtgagggaagaggtagcct | |
| | gctcacgtgcggtgatgtggaggagaaccctgggcctatgctctgtttgggctggatatttctttggcttgttg | |
| | caggagagcgaattaaaggatttaatatttcaggttgttccacaaaaaaactcctttggacatattctacaagg | |
| | agtgaagaggaatttgtcttattttgtgatttaccagagccacagaaatcacatttctgccacagaaatcgactc | |
| | tcaccaaaacaagtccctgagcacctgcccttcatgggtagtaacgacctatctgatgtccaatggtaccaa | |
| | caaccttcgaatggagatccattagaggacattaggaaaagctatcctcacatcattcaggacaaatgtacc | |
| | cttcactttttgaccccaggggtgaataattctgggtcatatatttgtagacccaagatgattaagagcccctat | |
| | gatgtagcctgttgtgtcaagatgattttagaagttaagccccagacaaatgcatcctgtgagtattccgcatc | |
| | acataagcaagacctacttcttgggagcactggctctatttcttgccccagtctcagctgccaaagtgatgca | |
| | caaagtccagcggtaacctggtacaagaatggaaaactcctctctgtggaaaggagcaaccgaatcgtagt | |
| | ggatgaagtttatgactatcaccagggcacatatgtatgtgattacactcagtcggatactgtgagttcgtgga | |
| | cagtcagagctgttgttcaagtgagaaccattgtgggagacactaaactcaaaccagatattctggatcctgt | |
| | cgaggacacactggaagtagaacttggaaagcctttaactattagctgcaaagcacgatttggctttgaaag | |
| | ggtctttaaccctgtcataaaatggtacatcaaagattctgacctagagtgggaagtctcagtacctgaggcg | |
| | aaaagtattaaatccactttaaaggatgaaatcattgagcgtaatatcatcttggaaaaagtcactcagcgtga | |
| | tcttcgcaggaagtttgtttgctttgtccagaactccattggaaacacaacccagtccgtccaactgaaagaa | |
| | aagagaggagtggtgctcctgtacatcctgcttggcaccatcgggaccctggtggccgtgctggcggcga | |
| | gtgccctcctctacaggcactggattgaaatagtgctgctgtaccggacctaccagagcaaggatcagacg | |
| | cttggggataaaaaggattttgatgctttcgtatcctatgcaaaatggagctcttttccaagtgaggccacttca | |
| | tctctgagtgaagaacacttggccctgagcctatttcctgatgttttagaaaacaaatatggatatagcctgtgt | |
| | ttgcttgaaagagatgtggctccaggaggagtgtatgcagaagacattgtgagcattattaagagaagcaga | |
| | agaggaatatttatcttgagccccaactatgtcaatggacccagtatctttgaactacaagcagcagtgaatct | |
| | tgccttggatgatcaaacactgaaactcattttaattaagttctgttacttccaagagccagagtctctacctcat | |
| | ctcgtgaaaaaagctctcagggttttgcccacagttacttggagaggcttaaaatcagttcctcccaattctag | |
| | gttctgggccaaaatgcgctaccacatgcctgtgaaaaactctcagggattcacgtggaaccagctcagaat | |
| | tacctctaggatttttcagtggaaaggactcagtagaacagaaaccactgggaggagctcccagcctaagg | |
| | aatggtgataa | |
| NFAT inducible | ggaggaaaaactgtttcatacagaaggcgtggaggaaaaactgtttcatacagaaggcgtggaggaaaaa | 33 |
| IL-18 receptor | ctgtttcatacagaaggcgtactagttaggcgtgtacggtgggaggcctatataagcagagctcgtttagtga | |
| alpha and beta, | accgtcagatcgcctggagacgccatccacgctgttttgacctccatagaagacaccgggaccgatccag | |
| .3XIL2NFAT | cctcgagagacccaatgctagccaccatgaattgtagagaattacccttgaccctttgggtgcttatatctgta | |
| mCMV IL18 | agcactgcagaatcttgtacttcacgtccccacattactgtggttgaaggggaacctttctatctgaaacattg | |
| receptor alpha- | ctcgtgttcacttgcacatgagattgaaacaaccaccaaaagctggtacaaaagcagtggatcacaggaac | |
| T2A-IL18Rb | atgtggagctgaacccaaggagttccagcagaattgctttgcatgattgtgttttggagttttggccagttgagt | |
| | tgaatgacacaggatcttactttttccaaatgaaaaattatactcagaaatggaaattaaatgtcatcagaagaa | |
| | ataaacacagctgtttcactgaaagacaagtaactagtaaaattgtggaagttaaaaaattttttcagataacct | |
| | gtgaaaacagttactatcaaacactggtcaacagcacatcattgtataagaactgtaaaaagctactactgga | |
| | gaacaataaaaacccaacgataaagaagaacgccgagtttgaagatcaggggtattactcctgcgtgcattt | |
| | ccttcatcataatggaaaactatttaatatcaccaaaaccttcaatataacaatagtggaagatcgcagtaatat | |
| | agttccggttcttcttggaccaaagcttaaccatgttgcagtggaattaggaaaaaacgtaaggctcaactgc | |
| | tctgctttgctgaatgaagaggatgtaatttattggatgttcggggaagaaaatggatcggatcctaatatacat | |
| | gaagagaaagaaatgagaattatgactccagaaggcaaatggcatgcttcaaaagtattgagaattgaaaat | |
| | attggtgaaagcaatctaaatgttttatataattgcactgtggccagcacgggaggcacagacaccaaaagc | |
| | ttcatcttggtgagaaaagcagacatggctgatatcccaggccacgtcttcacaagaggaatgatcatagct | |
| | gttttgatcttggtggcagtagtgtgcctagtgactgtgtgtgtcatttatagagttgacttggttctattttataga | |
| | catttaacgagaagagatgaaacattaacagatggaaaaacatatgatgcttttgtgtcttacctaaaagaatg | |
| | ccgacctgaaaatggagaggagcacacctttgctgtggagattttgcccagggtgttggagaaacattttgg | |
| | gtataagttatgcatatttgaaagggatgtagtgcctggaggagctgttgttgatgaaatccactcactgatag | |
| | agaaaagccgaagactaatcattgtcctaagtaaaagttatatgtctaatgaggtcaggtatgaacttgaaagt | |
| | ggactccatgaagcattggtggaaagaaaaattaaaataatcttaattgaatttacacctgttactgacttcaca | |
| | ttcttgccccaatcactaaagcttttgaaatctcacagagttctgaagtggaaggccgataaatctctttcttata | |
| | actcaaggttctggaagaaccttctttacttaatgcctgcaaaaacagtcaagccaggtagagacgaaccgg | |
| | aagtcttgcctgttctttccgagtctggaagcggtgagggaagaggtagcctgctcacgtgcggtgatgtgg | |
| | aggagaaccctgggcctatgctctgtttgggctggatatttctttggcttgttgcaggagagcgaattaaagg | |
| | atttaatatttcaggttgttccacaaaaaaactcctttggacatattctacaaggagtgaagaggaatttgtcttat | |
| | tttgtgatttaccagagccacagaaatcacatttctgccacagaaatcgactctcaccaaaacaagtccctga | |
| | gcacctgcccttcatgggtagtaacgacctatctgatgtccaatggtaccaacaaccttcgaatggagatcca | |
| | ttagaggacattaggaaaagctatcctcacatcattcaggacaaatgtacccttcactttttgaccccaggggt | |
| | gaataattctgggtcatatatttgtagacccaagatgattaagagcccctatgatgtagcctgttgtgtcaagat | |
| | gattttagaagttaagccccagacaaatgcatcctgtgagtattccgcatcacataagcaagacctacttcttg | |
| | ggagcactggctctatttcttgccccagtctcagctgccaaagtgatgcacaaagtccagcggtaacctggt | |
| | acaagaatggaaaactcctctctgtggaaaggagcaaccgaatcgtagtggatgaagtttatgactatcacc | |
| | agggcacatatgtatgtgattacactcagtcggatactgtgagttcgtggacagtcagagctgttgttcaagtg | |
| | agaaccattgtgggagacactaaactcaaaccagatattctggatcctgtcgaggacacactggaagtaga | |
| | acttggaaagcctttaactattagctgcaaagcacgatttggctttgaaagggtctttaaccctgtcataaaatg | |
| | gtacatcaaagattctgacctagagtgggaagtctcagtacctgaggcgaaaagtattaaatccactttaaag | |
| | gatgaaatcattgagcgtaatatcatcttggaaaaagtcactcagcgtgatcttcgcaggaagtttgtttgcttt | |
| | gtccagaactccattggaaacacaacccagtccgtccaactgaaagaaaagagaggagtggtgctcctgt | |
| | acatcctgcttggcaccatcgggaccctggtggccgtgctggcggcgagtgccctcctctacaggcactg | |
| | gattgaaatagtgctgctgtaccggacctaccagagcaaggatcagacgcttggggataaaaaggattttg | |
| | atgctttcgtatcctatgcaaaatggagctcttttccaagtgaggccacttcatctctgagtgaagaacacttgg | |
| | ccctgagcctatttcctgatgttttagaaaacaaatatggatatagcctgtgtttgcttgaaagagatgtggctc | |
| | caggaggagtgtatgcagaagacattgtgagcattattaagagaagcagaagaggaatatttatcttgagcc | |
| | ccaactatgtcaatggacccagtatctttgaactacaagcagcagtgaatcttgccttggatgatcaaacact | |
| | gaaactcattttaattaagttctgttacttccaagagccagagtctctacctcatctcgtgaaaaaagctctcag | |
| | ggttttgcccacagttacttggagaggcttaaaatcagttcctcccaattctaggttctgggccaaaatgcgct | |
| | accacatgcctgtgaaaaactctcagggattcacgtggaaccagctcagaattacctctaggatttttcagtg | |
| | gaaaggactcagtagaacagaaaccactgggaggagctcccagcctaaggaatggtgataa | |
| CD8 leader | MALPVTALLLPLALLLHAARP | 34 |
| sequence | | |
| PSMGFR peptide. | GTINVHDVETQFNQYKTEAASRYNLTISDVSVSDVPFPFSAQSGA | 35 |
| N delta10 portion | QFNQYKTEAASRYNLTISDVSVSDVPFPFSAQSGA | 36 |
| of PSMGFR | | |

Targeting Moiety

| | | |
|---|---|---|
| C2 HC-CDR1 | GYAMS | 37 |
| C2 HC-CDR2 | TISSGGTYIYYPDSVKG | |
| C2 HC-CDR3 | LGGDNYYEYFDV | 39 |
| C2 LC-CDR1 | RASKSVSTSGYSYMH | 40 |
| C2 LC-CDR2 | LASNLES | 41 |
| C2 LC-CDR3 | QHSRELPFT | 42 |
| C2 heavy chain | EVQLVESGGGLVKPGGSLRLSCAASGFTFSGYAMSWVRQAPGKGLEWVS | 43 |
| variable domain | TISSGGTYIYYPDSVKGRFTISRDNAKNSLYLQMNSLRAEDTAVYYCARLG | |
| | GDNYYEYFDVWGKGTTVTVSS | |
| C2 light chain | DIVLTQSPASLAVSPGQRATITCRASKSVSTSGYSYMHWYQQKPGQPPKLL | 44 |
| variable domain | IYLASNLESGVPARFSGSGSGTDFTLTINPVEANDTANYYCQHSRELPFTFG | |
| | GGTKVEIKRT | |
| C2 scFv | EVQLVESGGGLVKPGGSLRLSCAASGFTFSGYAMSWVRQAPGKGLEWVS | 45 |
| | TISSGGTYIYYPDSVKGRFTISRDNAKNSLYLQMNSLRAEDTAVYYCARLG | |
| | GDNYYEYFDVWGKGTTVTVSSGGGGSGGGGSGGGGSDIVLTQSPASLAV | |
| | SPGQRATITCRASKSVSTSGYSYMHWYQQKPGQPPKLLIYLASNLESGVPA | |
| | RFSGSGSGTDFTLTINPVEANDTANYYCQHSRELPFTFGGGTKVEIKRT | |
| E6 HC-CDR1 | RYGMS | 46 |
| E6 HC-CDR2 | TISGGGTYIYYPDSVKG | 47 |
| E6 HC-CDR3 | DNYGRNYDYGMDY | 48 |
| E6 LC-CDR1 | SATSSVSYIH | 49 |
| E6 LC-CDR2 | STSNLAS | 50 |
| E6 LC-CDR3 | QQRSSSPFT | 51 |
| E6 heavy chain | EVQLVESGGGLVKPGGSLRLSCAASGFTFSRYGMSWVRQAPGKRLEWVS | 52 |
| variable domain | TISGGGTYIYYPDSVKGRFTISRDNAKNTLYLQMNSLRAEDTAVYYCTRDN | |
| | YGRNYDYGMDYWGQGTLVTVSS | |
| E6 light chain | EIVLTQSPATLSLSPGERATLTCSATSSVSYIHWYQQRPGQSPRLLIYSTSNL | 53 |
| variable domain | ASGIPARFSGSGSGSDYTLTISSLEPEDFAVYYCQQRSSSPFTFGSGTKVEIK | |
| E6 scFv | EVQLVESGGGLVKPGGSLRLSCAASGFTFSRYGMSWVRQAPGKRLEWVS | 54 |
| | TISGGGTYIYYPDSVKGRFTISRDNAKNTLYLQMNSLRAEDTAVYYCTRDN | |
| | YGRNYDYGMDYWGQGTLVTVSSGGGGSGGGGSGGGGSEIVLTQSPATLS | |
| | LSPGERATLTCSATSSVSYIHWYQQRPGQSPRLLIYSTSNLASGIPARFSGSG | |
| | SGSDYTLTISSLEPEDFAVYYCQQRSSSPFTFGSGTKVEIK | |
| 20A10 HC-CDR1 | TYAMS | 55 |
| 20A10 HC-CDR2 | SIGRAGSTYYSDSVKG | 56 |
| 20A10 HC-CDR3 | GPIYNDYDEFAY | 57 |
| 20A10 LC-CDR1 | KSSQSVLYSSNQKNYLA | 58 |
| 20A10 LC-CDR2 | WASTRES | 59 |
| 20A10 LC-CDR3 | HQYLSSLT | 60 |
| 20A10 heavy chain | QVQLVESGGGLVKPGGSLRLSCAASGFTFSTYAMSWIRQAPGKGLEWVAS | 61 |
| variable domain | IGRAGSTYYSDSVKGRFTISRDNAKNSLYLQMNSLRAEDTAVYYCARGPIY | |
| | NDYDEFAYWGQGTLVTVSS | |
| 20A10 light chain | EIVLTQSPATLSLSPGERATLSCKSSQSVLYSSNQKNYLAWYQQKPGQAPR | 62 |
| variable domain | LLIYWASTRESGIPARFSGSGSGTDFTLTISSLEPEDFAVYYCHQYLSSLTFG | |
| | GGTKVEIKR | |
| 20A10 scFv | QVQLVESGGGLVKPGGSLRLSCAASGFTFSTYAMSWIRQAPGKGLEWVAS | 63 |
| | IGRAGSTYYSDSVKGRFTISRDNAKNSLYLQMNSLRAEDTAVYYCARGPIY | |
| | NDYDEFAYWGQGTLVTVSSGGGGSGGGGGGGGSEIVLTQSPATLSLSPG | |
| | ERATLSCKSSQSVLYSSNQKNYLAWYQQKPGQAPRLLIYWASTRESGIPAR | |
| | FSGSGSGTDFTLTISSLEPEDFAVYYCHQYLSSLTFGGGTKVEIKR | |

Hinge

| | | |
|---|---|---|
| CD8 hinge amino | TTTPAPRPPTPAPTIASQPLSLRPEACRPAAGGAVHTRGLDFACD | 64 |
| acid sequence | | |
| CD28 hinge amino | KHLCPSPLFPGPSKP | 65 |
| acid sequence | | |

Transmembrane

| | | |
|---|---|---|
| CD8 | IYIWAPLAGTCGVLLLSLVITLYC | 66 |
| Transmembrane | | |
| amino acid | | |
| sequence | | |
| CD28 | FWVLVVVGGVLACYSLLVTVAFIIFWV | 67 |
| Transmembrane | | |
| amino acid | | |
| sequence | | |

Co-stimulatory

| | | |
|---|---|---|
| 41BB | KRGRKKLLYIFKQPFMRPVQTTQEEDGCSCRFPEEEEGGCEL | 68 |
| Costimulatory | | |
| amino acid | | |
| sequence | | |
| CD28 | RSKRSRLLHSDYMNMTPRRPGPTRKHYQPYAPPRDFAAYRS | 69 |
| Costimulatory | | |
| amino acid | | |
| sequence | | |

Signaling

| CD3ζ Signaling | | RVKFSRSADAPAYKQGQNQLYNELNLGRREEYDVLDKRRGRDPEMGGKP | 70 |
|---|---|---|---|
| amino acid | | RRKNPQEGLYNELQKDKMAEAYSEIGMKGERRRGKGHDGLYQGLSTATK | |
| sequence. | | DTYDALHMQALPPR | |
| CD3ζ 1XX | | RVKFSRSADAPAYKQGQNQLYNELNLGRREEYDVLDKRRGRDPEMGGKP | 71 |
| Signaling amino | | RRKNPQEGLFNELQKDKMAEAFSEIGMKGERRRGKGHDGLFQGLSTATK | |
| acid sequence. | | DTFDALHMQALPPR | |
| C2 | CD8- | MALPVTALLLPLALLLHAARPEVQLVESGGGLVKPGGSLRLSCAAS | 72 |
| | CD8- | GFTFSGYAMSWVRQAPGKGLEWVSTISSGGTYIYYPDSVKGRFTIS | |
| | 41BB | RDNAKNSLYLQMNSLRAEDTAVYYCARLGGDNYYEYFDVWGKG | |
| | CD3 | TTVTVSSGGGGGGGGSGGGGSDIVLTQSPASLAVSPGQRATITCR | |
| | | ASKSVSTSGYSYMHWYQQKPGQPPKLLIYLASNLESGVPARFSGSG | |
| | | SGTDFTLTINPVEANDTANYYCQHSRELPFTFGGGTKVEIKRTTTTP | |
| | | APRPPTPAPTIASQPLSLRPEACRPAAGGAVHTRGLDFACDIYIWAP | |
| | | LAGTCGVLLLSLVITLYCKRGRKKLLYIFKQPFMRPVQTTQEEDGC | |
| | | SCRFPEEEEGGCELRVKFSRSADAPAYKQGQNQLYNELNLGRREEY | |
| | | DVLDKRRGRDPEMGGKPRRKNPQEGLYNELQKDKMAEAYSEIGM | |
| | | KGERRRGKGHDGLYQGLSTATKDTYDALHMQALPPR | |
| E6 | CD8- | MALPVTALLLPLALLLHAARPEVQLVESGGGLVKPGGSLRLSCAAS | 73 |
| | CD8- | GFTFSRYGMSWVRQAPGKRLEWVSTISGGGTYIYYPDSVKGRFTIS | |
| | 41BB | RDNAKNTLYLQMNSLRAEDTAVYYCTRDNYGRNYDYGMDYWGQ | |
| | CD3 | GTLVTVSSGGGGSGGGGSGGGGSEIVLTQSPATLSLSPGERATLTCS | |
| | | ATSSVSYIHWYQQRPGQSPRLLIYSTSNLASGIPARFSGSGSGSDYTL | |
| | | TISSLEPEDFAVYYCQQRSSSPFTFGSGTKVEIKTTTPAPRPPTPAPTI | |
| | | ASQPLSLRPEACRPAAGGAVHTRGLDFACDIYIWAPLAGTCGVLLL | |
| | | SLVITLYCKRGRKKLLYIFKQPFMRPVQTTQEEDGCSCRFPEEEEGG | |
| | | CELRVKFSRSADAPAYKQGQNQLYNELNLGRREEYDVLDKRRGRD | |
| | | PEMGGKPRRKNPQEGLYNELQKDKMAEAYSEIGMKGERRRGKGH | |
| | | DGLYQGLSTATKDTYDALHMQALPPR | |
| 20A10N | CD8- | MALPVTALLLPLALLLHAARPQVQLVESGGGLVKPGGSLRLSCAAS | 74 |
| | CD8- | GFTFSTYAMSWIRQAPGKGLEWVASIGRAGSTYYSDSVKGRFTISR | |
| | 41BB | DNAKNSLYLQMNSLRAEDTAVYYCARGPIYNDYDEFAYWGQGTL | |
| | CD3 | VTVSSGGGGSGGGGSGGGGSEIVLTQSPATLSLSPGERATLSCKSSQ | |
| | | SVLYSSNQKNYLAWYQQKPGQAPRLLIYWASTRESGIPARFSGSGS | |
| | | GTDFTLTISSLEPEDFAVYYCHQYLSSLTFGGGTKVEIKRTTTPAPRP | |
| | | PTPAPTIASQPLSLRPEACRPAAGGAVHTRGLDFACDIYIWAPLAGT | |
| | | CGVLLLSLVITLYCKRGRKKLLYIFKQPFMRPVQTTQEEDGCSCRFP | |
| | | EEEEGGCELRVKFSRSADAPAYKQGQNQLYNELNLGRREEYDVLD | |
| | | KRRGRDPEMGGKPRRKNPQEGLYNELQKDKMAEAYSEIGMKGER | |
| | | RRGKGHDGLYQGLSTATKDTYDALHMQALPPR | |
| C2 | CD28- | MALPVTALLLPLALLLHAARPEVQLVESGGGLVKPGGSLRLSCAAS | 75 |
| | CD28- | GFTFSGYAMSWVRQAPGKGLEWVSTISSGGTYIYYPDSVKGRFTIS | |
| | CD28 | RDNAKNSLYLQMNSLRAEDTAVYYCARLGGDNYYEYFDVWGKG | |
| | CD3 | TTVTVSSGGGGSGGGGSGGGGSDIVLTQSPASLAVSPGQRATITCR | |
| | | ASKSVSTSGYSYMHWYQQKPGQPPKLLIYLASNLESGVPARFSGSG | |
| | | SGTDFTLTINPVEANDTANYYCQHSRELPFTFGGGTKVEIKRTKHLC | |
| | | PSPLFPGPSKPFWVLVVVGGVLACYSLLVTVAFIIFWVRSKRSRLLH | |
| | | SDYMNMTPRRPGPTRKHYQPYAPPRDFAAYRSRVKFSRSADAPAY | |
| | | KQGQNQLYNELNLGRREEYDVLDKRRGRDPEMGGKPRRKNPQEG | |
| | | LYNELQKDKMAEAYSEIGMKGERRRGKGHDGLYQGLSTATKDTY | |
| | | DALHMQALPPR | |
| E6 | CD28- | MALPVTALLLPLALLLHAARPEVQLVESGGGLVKPGGSLRLSCAAS | 76 |
| | CD28- | GFTFSRYGMSWVRQAPGKRLEWVSTISGGGTYIYYPDSVKGRFTIS | |
| | CD28 | RDNAKNTLYLQMNSLRAEDTAVYYCTRDNYGRNYDYGMDYWGQ | |
| | CD3 | GTLVTVSSGGGGSGGGGSGGGGSEIVLTQSPATLSLSPGERATLTCS | |
| | | ATSSVSYIHWYQQRPGQSPRLLIYSTSNLASGIPARFSGSGSGSDYTL | |
| | | TISSLEPEDFAVYYCQQRSSSPFTFGSGTKVEIKKHLCPSPLFPGPSKP | |
| | | FWVLVVVGGVLACYSLLVTVAFIIFWVRSKRSRLLHSDYMNMTPR | |
| | | RPGPTRKHYQPYAPPRDFAAYRSRVKFSRSADAPAYKQGQNQLYN | |
| | | ELNLGRREEYDVLDKRRGRDPEMGGKPRRKNPQEGLYNELQKDK | |
| | | MAEAYSEIGMKGERRRGKGHDGLYQGLSTATKDTYDALHMQALP | |
| | | PR | |
| 20A10N | CD28- | MALPVTALLLPLALLLHAARPQVQLVESGGGLVKPGGSLRLSCAAS | 77 |
| | CD28- | GFTFSTYAMSWIRQAPGKGLEWVASIGRAGSTYYSDSVKGRFTISR | |
| | CD28 | DNAKNSLYLQMNSLRAEDTAVYYCARGPIYNDYDEFAYWGQGTL | |
| | CD3 | VTVSSGGGGSGGGGSGGGGSEIVLTQSPATLSLSPGERATLSCKSSQ | |
| | | SVLYSSNQKNYLAWYQQKPGQAPRLLIYWASTRESGIPARFSGSGS | |
| | | GTDFTLTISSLEPEDFAVYYCHQYLSSLTFGGGTKVEIKRKHLCPSPL | |
| | | FPGPSKPFWVLVVVGGVLACYSLLVTVAFIIFWVRSKRSRLLHSDY | |
| | | MNMTPRRPGPTRKHYQPYAPPRDFAAYRSRVKFSRSADAPAYKQG | |
| | | QNQLYNELNLGRREEYDVLDKRRGRDPEMGGKPRRKNPQEGLYN | |
| | | ELQKDKMAEAYSEIGMKGERRRGKGHDGLYQGLSTATKDTYDAL | |
| | | HMQALPPR | |
| C2 | CD8- | MALPVTALLLPLALLLHAARPEVQLVESGGGLVKPGGSLRLSCAAS | 78 |
| | CD8- | GFTFSGYAMSWVRQAPGKGLEWVSTISSGGTYIYYPDSVKGRFTIS | |
| | 41BB | RDNAKNSLYLQMNSLRAEDTAVYYCARLGGDNYYEYFDVWGKG | |
| | 1XX | TTVTVSSGGGGSGGGGSGGGGSDIVLTQSPASLAVSPGQRATITCR | |
| | | ASKSVSTSGYSYMHWYQQKPGQPPKLLIYLASNLESGVPARFSGSG | |
| | | SGTDFTLTINPVEANDTANYYCQHSRELPFTFGGGTKVEIKRTTTTP | |
| | | APRPPTPAPTIASQPLSLRPEACRPAAGGAVHTRGLDFACDIYIWAP | |
| | | LAGTCGVLLLSLVITLYCKRGRKKLLYIFKQPFMRPVQTTQEEDGC | |
| | | SCRFPEEEEGGCELRVKFSRSADAPAYKQGQNQLYNELNLGRREEY | |
| | | DVLDKRRGRDPEMGGKPRRKNPQEGLFNELQKDKMAEAFSEIGM | |
| | | KGERRRGKGHDGLFQGLSTATKDTFDALHMQALPPR | |
| E6 | CD8- | MALPVTALLLPLALLLHAARPEVQLVESGGGLVKPGGSLRLSCAAS | 79 |
| | CD8- | GFTFSRYGMSWVRQAPGKRLEWVSTISGGGTYIYYPDSVKGRFTIS | |
| | 41BB | RDNAKNTLYLQMNSLRAEDTAVYYCTRDNYGRNYDYGMDYWGQ | |
| | 1XX | GTLVTVSSGGGGSGGGGSGGGGSEIVLTQSPATLSLSPGERATLTCS | |
| | | ATSSVSYIHWYQQRPGQSPRLLIYSTSNLASGIPARFSGSGSGSDYTL | |
| | | TISSLEPEDFAVYYCQQRSSSPFTFGSGTKVEIKTTTPAPRPPTPAPTI | |
| | | ASQPLSLRPEACRPAAGGAVHTRGLDFACDIYIWAPLAGTCGVLLL | |
| | | SLVITLYCKRGRKKLLYIFKQPFMRPVQTTQEEDGCSCRFPEEEEGG | |
| | | CELRVKFSRSADAPAYKQGQNQLYNELNLGRREEYDVLDKRRGRD | |
| | | PEMGGKPRRKNPQEGLFNELQKDKMAEAFSEIGMKGERRRGKGH | |
| | | DGLFQGLSTATKDTFDALHMQALPPR | |
| 20A10N | CD8- | MALPVTALLLPLALLLHAARPQVQLVESGGGLVKPGGSLRLSCAAS | 80 |
| | CD8- | GFTFSTYAMSWIRQAPGKGLEWVASIGRAGSTYYSDSVKGRFTISR | |
| | 41BB | DNAKNSLYLQMNSLRAEDTAVYYCARGPIYNDYDEFAYWGQGTL | |
| | 1XX | VTVSSGGGGSGGGGSGGGGSEIVLTQSPATLSLSPGERATLSCKSSQ | |
| | | SVLYSSNQKNYLAWYQQKPGQAPRLLIYWASTRESGIPARFSGSGS | |
| | | GTDFTLTISSLEPEDFAVYYCHQYLSSLTFGGGTKVEIKRTTTPAPRP | |
| | | PTPAPTIASQPLSLRPEACRPAAGGAVHTRGLDFACDIYIWAPLAGT | |
| | | CGVLLLSLVITLYCKRGRKKLLYIFKQPFMRPVQTTQEEDGCSCRFP | |
| | | EEEEGGCELRVKFSRSADAPAYKQGQNQLYNELNLGRREEYDVLD | |
| | | KRRGRDPEMGGKPRRKNPQEGLFNELQKDKMAEAFSEIGMKGERR | |
| | | RGKGHDGLFQGLSTATKDTFDALHMQALPPR | |
| C2 | CD28- | MALPVTALLLPLALLLHAARPEVQLVESGGGLVKPGGSLRLSCAAS | 81 |
| | CD28- | GFTFSGYAMSWVRQAPGKGLEWVSTISSGGTYIYYPDSVKGRFTIS | |
| | CD28 | RDNAKNSLYLQMNSLRAEDTAVYYCARLGGDNYYEYFDVWGKG | |
| | 1XX | TTVTVSSGGGGSGGGGSGGGGSDIVLTQSPASLAVSPGQRATITCR | |
| | | ASKSVSTSGYSYMHWYQQKPGQPPKLLIYLASNLESGVPARFSGSG | |
| | | SGTDFTLTINPVEANDTANYYCQHSRELPFTFGGGTKVEIKRTKHLC | |
| | | PSPLFPGPSKPFWVLVVVGGVLACYSLLVTVAFIIFWVRSKRSRLLH | |
| | | SDYMNMTPRRPGPTRKHYQPYAPPRDFAAYRSRVKFSRSADAPAY | |
| | | KQGQNQLYNELNLGRREEYDVLDKRRGRDPEMGGKPRRKNPQEG | |
| | | LFNELQKDKMAEAFSEIGMKGERRRGKGHDGLFQGLSTATKDTFD | |
| | | ALHMQALPPR | |
| E6 | CD28- | MALPVTALLLPLALLLHAARPEVQLVESGGGLVKPGGSLRLSCAAS | 82 |
| | CD28- | GFTFSRYGMSWVRQAPGKRLEWVSTISGGGTYIYYPDSVKGRFTIS | |
| | CD28 | RDNAKNTLYLQMNSLRAEDTAVYYCTRDNYGRNYDYGMDYWGQ | |
| | 1XX | GTLVTVSSGGGGSGGGGSGGGGSEIVLTQSPATLSLSPGERATLTCS | |
| | | ATSSVSYIHWYQQRPGQSPRLLIYSTSNLASGIPARFSGSGSGSDYTL | |
| | | TISSLEPEDFAVYYCQQRSSSPFTFGSGTKVEIKKHLCPSPLFPGPSKP | |
| | | FWVLVVVGGVLACYSLLVTVAFIIFWVRSKRSRLLHSDYMNMTPR | |
| | | RPGPTRKHYQPYAPPRDFAAYRSRVKFSRSADAPAYKQGQNQLYN | |
| | | ELNLGRREEYDVLDKRRGRDPEMGGKPRRKNPQEGLFNELQKDK | |
| | | MAEAFSEIGMKGERRRGKGHDGLFQGLSTATKDTFDALHMQALPP | |
| | | R | |
| 20A10N | CD28- | MALPVTALLLPLALLLHAARPQVQLVESGGGLVKPGGSLRLSCAAS | 83 |
| | CD28- | GFTFSTYAMSWIRQAPGKGLEWVASIGRAGSTYYSDSVKGRFTISR | |
| | CD28 | DNAKNSLYLQMNSLRAEDTAVYYCARGPIYNDYDEFAYWGQGTL | |
| | 1XX | VTVSSGGGGSGGGGSGGGGSEIVLTQSPATLSLSPGERATLSCKSSQ | |
| | | SVLYSSNQKNYLAWYQQKPGQAPRLLIYWASTRESGIPARFSGSGS | |
| | | GTDFTLTISSLEPEDFAVYYCHQYLSSLTFGGGTKVEIKRKHLCPSPL | |
| | | FPGPSKPFWVLVVVGGVLACYSLLVTVAFIIFWVRSKRSRLLHSDY | |
| | | MNMTPRRPGPTRKHYQPYAPPRDFAAYRSRVKFSRSADAPAYKQG | |
| | | QNQLYNELNLGRREEYDVLDKRRGRDPEMGGKPRRKNPQEGLFNE | |
| | | LQKDKMAEAFSEIGMKGERRRGKGHDGLFQGLSTATKDTFDALH | |
| | | MQALPPR | |
| C2 | CD8- | atggccttaccagtgaccgccttgctcctgccgctggccttgctgctccacgccgccaggccggaggtgca | 84 |
| | CD8- | gctggtggagtctgggggaggcctggtcaagcctggggggtccctgagactctcctgtgcagcctctgga | |
| | 41BB | ttcaccttcagtggctatgccatgagctgggtccgccaggctccagggaaggggctggagtgggtctcaac | |
| | CD3 | cattagtagtggcggaacctacatatactaccccgactcagtgaagggccgattcaccatctccagagaca | |
| | | acgccaagaactcactgtatctgcaaatgaacagcctgagagccgaggacacggccgtgtattactgtgcg | |
| | | agacttgggggggataattactacgaatacttcgatgtctggggcaaagggaccacggtcaccgtctcctc | |
| | | cggcggtggcggatccggcggtggcggatccggcggtggcggatccgacattgtgctgacccagtctcc | |
| | | agcctccttggccgtgtctccaggacagagggccaccatcacctgcagagccagtaagagtgtcagtacc | |
| | | agcggatactcctacatgcactggtatcagcagaaaccaggacaacctcctaaactcctgatttacctggca | |
| | | tccaatctggagagcggggtcccagccaggttcagcggcagtgggtctgggaccgatttcaccctcacaat | |
| | | taatcctgtggaagctaatgatactgcaaattattactgtcagcacagtagggagctgcctttcacattcggcg | |
| | | gagggaccaaggtggagatcaaacgaactacaacaacccctgcccccagacctcctaccccagccccta | |
| | | caattgccagccagcctctgagcctgaggcccgaggcttgtagacctgctgctggcggagccgtgcacac | |
| | | cagaggactggatttcgcctgcgacatctacatctgggcgcccttggccgggacttgtggggtccttctcct | |
| | | gtcactggttatcaccctttactgcaaacggggcagaaagaaactcctgtatatattcaaacaaccatttatga | |
| | | gaccagtacaaactactcaagaggaagatggctgtagctgccgatttccagaagaagaagaaggaggatg | |
| | | tgaactgagagtgaagttcagcaggagcgcagacgcccccgcgtacaagcagggccagaaccagctct | |
| | | ataacgagctcaatctaggacgaagagaggagtacgatgttttggacaagagacgtggccgggaccctga | |
| | | gatggggggaaagccgagaaggaagaaccctcaggaaggcctgtacaatgaactgcagaaagataaga | |
| | | tggcggaggcctacagtgagattgggatgaaaggcgagcgccggaggggcaaggggcacgatggcctt | |
| | | taccagggtctcagtacagccaccaaggacacctacgacgcccttcacatgcaggccctgccccctcgc | |
| E6 | CD8- | atggccttaccagtgaccgccttgctcctgccgctggccttgctgctccacgccgccaggccggaggtgca | 85 |
| | CD8- | gctggtggagtctgggggaggcctggtcaagcctggggggtccctgagactctcctgtgcagcctctgga | |
| | 41BB | ttcaccttcagtaggtatggcatgagctgggtccgccaggctccagggaagaggctggagtgggtctcaac | |
| | CD3 | cattagtggcggaggcacctacatatactacccagactcagtgaagggccgattcaccatctccagagaca | |
| | | acgccaagaacaccctgtatctgcaaatgaacagcctgagagccgaggacacggctgtgtattactgtacc | |
| | | agagataactatggccgcaactatgattatggcatggattattggggccagggcaccctggtgaccgtgag | |
| | | cagcggtggtggcggttccggcggtggcggttccggtggtggcggttccgaaattgtgttgacacagtctc | |
| | | cagccaccctgtctttgtctccaggggaaagagccaccctcacctgcagcgccaccagcagtgttagctac | |
| | | atccactggtatcaacagaggcctggccagagccccaggctcctcatctatagcacctccaacctggccag | |
| | | cggcatcccagccaggttcagtggcagtgggtctgggagcgactacactctcaccatcagcagcctagag | |
| | | cctgaagattttgcagtttattactgtcagcagcgtagcagctcccctttcacctttggcagcggcaccaaagt | |
| | | ggaaattaaaacaacaacccctgcccccagacctcctaccccagcccctacaattgccagccagcctctga | |
| | | gcctgaggcccgaggcttgtagacctgctgctggcggagccgtgcacaccagaggactggatttcgcctg | |
| | | cgacatctacatctgggcgcccttggccgggacttgtggggtccttctcctgtcactggttatcaccctttact | |
| | | gcaaacggggcagaaagaaactcctgtatatattcaaacaaccatttatgagaccagtacaaactactcaag | |
| | | aggaagatggctgtagctgccgatttccagaagaagaagaaggaggatgtgaactgagagtgaagttcag | |
| | | caggagcgcagacgcccccgcgtacaagcagggccagaaccagctctataacgagctcaatctaggac | |
| | | gaagagaggagtacgatgttttggacaagagacgtggccgggaccctgagatggggggaaagccgaga | |
| | | aggaagaaccctcaggaaggcctgtacaatgaactgcagaaagataagatggcggaggcctacagtgag | |
| | | attgggatgaaaggcgagcgccggaggggcaaggggcacgatggcctttaccagggtctcagtacagc | |
| | | caccaaggacacctacgacgcccttcacatgcaggccctgccccctcgc | |
| 20A10N | CD8- | atggccttaccagtgaccgccttgctcctgccgctggccttgctgctccacgccgccaggccgcaggtgca | 86 |
| | CD8- | gctggttgaatctggcggcggacttgtgaagcctggcggatctctgagactgagctgtgccgccagcggct | |
| | 41BB | tcacctttagcacatacgccatgagctggatcagacaggcccctggcaaaggcctggaatgggggcgtct | |
| | CD3 | attggcagagccggcagcacctactacagcgactctgtgaagggcagattcaccatcagccgggacaac | |
| | | gccaagaacagcctgtacctgcagatgaactccctgagagccgaggacaccgccgtgtactattgtgcca | |
| | | gaggacccatctacaacgactacgacgagttcgcctattggggccagggcacactggtcacagtttctagc | |
| | | ggcggtggcggaagcggaggcggtggctccggtggcggaggcagcgaaattgtgctgacacagagcc | |
| | | ccgccacactgtcactttctccaggcgaaagagccacactgagctgcaagagcagccagagcgtgctgta | |
| | | ctccagcaaccagaagaactacctggcctggtatcagcagaagcccggccaagctcctcggctgctgatc | |
| | | tattgggccagcacaagagagagcggcatccctgccagattttctggcagcggctctggcaccgatttcac | |
| | | cctgaccataagcagcctggaacctgaggacttcgccgtgtattactgccaccagtacctgagcagcctga | |
| | | cctttggcggaggcaccaaggtggaaatcaagcggacaacaacccctgcccccagacctcctaccccag | |
| | | cccctacaattgccagccagcctctgagcctgaggcccgaggcttgtagacctgctgctggcggagccgt | |
| | | gcacaccagaggactggatttcgcctgcgacatctacatctgggcgcccttggccgggacttgtggggtcc | |
| | | ttctcctgtcactggttatcaccctttactgcaaacggggcagaaagaaactcctgtatatattcaaacaaccat | |
| | | ttatgagaccagtacaaactactcaagaggaagatggctgtagctgccgatttccagaagaagaagaagga | |
| | | ggatgtgaactgagagtgaagttcagcaggagcgcagacgcccccgcgtacaagcagggccagaacca | |
| | | gctctataacgagctcaatctaggacgaagagaggagtacgatgttttggacaagagacgtggccgggac | |
| | | cctgagatggggggaaagccgagaaggaagaaccctcaggaaggcctgtacaatgaactgcagaaaga | |
| | | taagatggcggaggcctacagtgagattgggatgaaaggcgagcgccggaggggcaaggggcacgat | |
| | | ggcctttaccagggtctcagtacagccaccaaggacacctacgacgcccttcacatgcaggccctgcccc | |
| | | ctcgc | |
| C2 | CD28- | atggccttaccagtgaccgccttgctcctgccgctggccttgctgctccacgccgccaggccggaggtgca | 87 |
| | CD28- | gctggtggagtctgggggaggcctggtcaagcctggggggtccctgagactctcctgtgcagcctctgga | |
| | CD28 | ttcaccttcagtggctatgccatgagctgggtccgccaggctccagggaaggggctggagtgggtctcaac | |
| | CD3 | cattagtagtggcggaacctacatatactaccccgactcagtgaagggccgattcaccatctccagagaca | |
| | | acgccaagaactcactgtatctgcaaatgaacagcctgagagccgaggacacggccgtgtattactgtgcg | |
| | | agacttgggggggataattactacgaatacttcgatgtctggggcaaagggaccacggtcaccgtctcctc | |
| | | cggcggtggcggatccggcggtggcggatccggcggtggcggatccgacattgtgctgacccagtctcc | |
| | | agcctccttggccgtgtctccaggacagagggccaccatcacctgcagagccagtaagagtgtcagtacc | |
| | | agcggatactcctacatgcactggtatcagcagaaaccaggacaacctcctaaactcctgatttacctggca | |
| | | tccaatctggagagcggggtcccagccaggttcagcggcagtgggtctgggaccgatttcaccctcacaat | |
| | | taatcctgtggaagctaatgatactgcaaattattactgtcagcacagtagggagctgcctttcacattcggcg | |
| | | gagggaccaaggtggagatcaaacgaactaagcacctgtgtccttctccactgttccccggccctagcaag | |
| | | cctttctgggtcctggtggtggtgggcggagtgctggcctgctacagcctgctggtgaccgtggcctttatca | |
| | | tcttctgggtgaggagtaagaggagcaggctcctgcacagtgactacatgaacatgactcctagaagacct | |
| | | gggcctaccagaaagcattaccagccctatgccccaccacgcgacttcgcagcctatcgctccagagtga | |
| | | agttcagcaggagcgcagacgcccccgcgtacaagcagggccagaaccagctctataacgagctcaatc | |
| | | taggacgaagagaggagtacgatgttttggacaagagacgtggccgggaccctgagatggggggaaag | |
| | | ccgagaaggaagaaccctcaggaaggcctgtacaatgaactgcagaaagataagatggcggaggccta | |
| | | cagtgagattgggatgaaaggcgagcgccggaggggcaaggggcacgatggcctttaccagggtctca | |
| | | gtacagccaccaaggacacctacgacgcccttcacatgcaggccctgccccctcgc | |
| E6 | CD28- | atggccttaccagtgaccgccttgctcctgccgctggccttgctgctccacgccgccaggccggaggtgca | 88 |
| | CD28- | gctggtggagtctgggggaggcctggtcaagcctggggggtccctgagactctcctgtgcagcctctgga | |
| | CD28 | ttcaccttcagtaggtatggcatgagctgggtccgccaggctccagggaagaggctggagtgggtctcaac | |
| | CD3 | cattagtggcggaggcacctacatatactacccagactcagtgaagggccgattcaccatctccagagaca | |
| | | acgccaagaacaccctgtatctgcaaatgaacagcctgagagccgaggacacggctgtgtattactgtacc | |
| | | agagataactatggccgcaactatgattatggcatggattattggggccagggcaccctggtgaccgtgag | |
| | | cagcggtggtggcggttccggcggtggcggttccggtggtggcggttccgaaattgtgttgacacagtctc | |
| | | cagccaccctgtctttgtctccaggggaaagagccaccctcacctgcagcgccaccagcagtgttagctac | |
| | | atccactggtatcaacagaggcctggccagagccccaggctcctcatctatagcacctccaacctggccag | |
| | | cggcatcccagccaggttcagtggcagtgggtctgggagcgactacactctcaccatcagcagcctagag | |
| | | cctgaagattttgcagtttattactgtcagcagcgtagcagctcccctttcacctttggcagcggcaccaaagt | |
| | | ggaaattaaaaagcacctgtgtccttctccactgttccccggccctagcaagcctttctgggtcctggtggtg | |
| | | gtgggcggagtgctggcctgctacagcctgctggtgaccgtggcctttatcatcttctgggtgaggagtaag | |
| | | aggagcaggctcctgcacagtgactacatgaacatgactcctagaagacctgggcctaccagaaagcatt | |
| | | accagccctatgccccaccacgcgacttcgcagcctatcgctccagagtgaagttcagcaggagcgcaga | |
| | | cgcccccgcgtacaagcagggccagaaccagctctataacgagctcaatctaggacgaagagaggagta | |
| | | cgatgttttggacaagagacgtggccgggaccctgagatggggggaaagccgagaaggaagaaccctc | |
| | | aggaaggcctgtacaatgaactgcagaaagataagatggcggaggcctacagtgagattgggatgaaag | |
| | | gcgagcgccggaggggcaaggggcacgatggcctttaccagggtctcagtacagccaccaaggacacc | |
| | | tacgacgcccttcacatgcaggccctgccccctcgc | |
| 20A10N | CD28- | atggccttaccagtgaccgccttgctcctgccgctggccttgctgctccacgccgccaggccgcaggtgca | 89 |
| | CD28- | gctggttgaatctggcggcggacttgtgaagcctggcggatctctgagactgagctgtgccgccagcggct | |
| | CD28 | tcacctttagcacatacgccatgagctggatcagacaggcccctggcaaaggcctggaatgggtggcgtct | |
| | CD3 | attggcagagccggcagcacctactacagcgactctgtgaagggcagattcaccatcagccgggacaac | |
| | | gccaagaacagcctgtacctgcagatgaactccctgagagccgaggacaccgccgtgtactattgtgcca | |
| | | gaggacccatctacaacgactacgacgagttcgcctattggggccagggcacactggtcacagtttctagc | |
| | | ggcggtggcggaagcggaggcggtggctccggtggcggaggcagcgaaattgtgctgacacagagcc | |
| | | ccgccacactgtcactttctccaggcgaaagagccacactgagctgcaagagcagccagagcgtgctgta | |
| | | ctccagcaaccagaagaactacctggcctggtatcagcagaagcccggccaagctcctcggctgctgatc | |
| | | tattgggccagcacaagagagagcggcatccctgccagattttctggcagcggctctggcaccgatttcac | |
| | | cctgaccataagcagcctggaacctgaggacttcgccgtgtattactgccaccagtacctgagcagcctga | |
| | | cctttggcggaggcaccaaggtggaaatcaagcggaagcacctgtgtccttctccactgttccccggccct | |
| | | agcaagcctttctgggtcctggtggtggtgggcggagtgctggcctgctacagcctgctggtgaccgtggc | |
| | | ctttatcatcttctgggtgaggagtaagaggagcaggctcctgcacagtgactacatgaacatgactcctag | |
| | | aagacctgggcctaccagaaagcattaccagccctatgccccaccacgcgacttcgcagcctatcgctcca | |
| | | gagtgaagttcagcaggagcgcagacgcccccgcgtacaagcagggccagaaccagctctataacgag | |
| | | ctcaatctaggacgaagagaggagtacgatgttttggacaagagacgtggccgggaccctgagatgggg | |
| | | ggaaagccgagaaggaagaaccctcaggaaggcctgtacaatgaactgcagaaagataagatggcgga | |
| | | ggcctacagtgagattgggatgaaaggcgagcgccggaggggcaaggggcacgatggcctttaccagg | |
| | | gtctcagtacagccaccaaggacacctacgacgcccttcacatgcaggccctgccccctcgc | |
| E6 | CD8- | atggccttaccagtgaccgccttgctcctgccgctggccttgctgctccacgccgccaggccggaggtgca | 90 |
| | CD8- | gctggtggagtctgggggaggcctggtcaagcctggggggtccctgagactctcctgtgcagcctctgga | |
| | 41BB | ttcaccttcagtggctatgccatgagctgggtccgccaggctccagggaaggggctggagtgggtctcaac | |
| | 1XX | cattagtagtggcggaacctacatatactaccccgactcagtgaagggccgattcaccatctccagagaca | |
| | | acgccaagaactcactgtatctgcaaatgaacagcctgagagccgaggacacggccgtgtattactgtgcg | |
| | | agacttgggggggataattactacgaatacttcgatgtctggggcaaagggaccacggtcaccgtctcctc | |
| | | cggcggtggcggatccggcggtggcggatccggcggtggcggatccgacattgtgctgacccagtctcc | |
| | | agcctccttggccgtgtctccaggacagagggccaccatcacctgcagagccagtaagagtgtcagtacc | |
| | | agcggatactcctacatgcactggtatcagcagaaaccaggacaacctcctaaactcctgatttacctggca | |
| | | tccaatctggagagcggggtcccagccaggttcagcggcagtgggtctgggaccgatttcaccctcacaat | |
| | | taatcctgtggaagctaatgatactgcaaattattactgtcagcacagtagggagctgcctttcacattcggcg | |
| | | gagggaccaaggtggagatcaaacgaactacaacaacccctgcccccagacctcctaccccagccccta | |
| | | caattgccagccagcctctgagcctgaggcccgaggcttgtagacctgctgctggcggagccgtgcacac | |
| | | cagaggactggatttcgcctgcgacatctacatctgggcgcccttggccgggacttgtggggtccttctcct | |
| | | gtcactggttatcaccctttactgcaaacggggcagaaagaaactcctgtatatattcaaacaaccatttatga | |
| | | gaccagtacaaactactcaagaggaagatggctgtagctgccgatttccagaagaagaagaaggaggatg | |
| | | tgaactgagagtgaagttcagcaggagcgcagacgcccccgcgtacaagcagggccagaaccagctct | |
| | | ataacgagctcaatctaggacgaagagaggagtacgatgttttggacaagagacgtggccgggaccctga | |
| | | gatggggggaaagccgagaaggaagaaccctcaggaaggcctgttcaatgaactgcagaaagataagat | |
| | | ggcggaggccttcagtgagattgggatgaaaggcgagcgccggaggggcaaggggcacgatggccttt | |
| | | tccagggtctcagtacagccaccaaggacaccttcgacgcccttcacatgcaggccctgccccctcgc | |
| C2 | CD8- | atggccttaccagtgaccgccttgctcctgccgctggccttgctgctccacgccgccaggccggaggtgca | 91 |
| | CD8- | gctggtggagtctgggggaggcctggtcaagcctggggggtccctgagactctcctgtgcagcctctgga | |
| | 41BB | ttcaccttcagtaggtatggcatgagctgggtccgccaggctccagggaagaggctggagtgggtctcaac | |
| | 1XX | cattagtggggaggcacctacatatactacccagactcagtgaagggccgattcaccatctccagagaca | |
| | | acgccaagaacaccctgtatctgcaaatgaacagcctgagagccgaggacacggctgtgtattactgtacc | |
| | | agagataactatggccgcaactatgattatggcatggattattggggccagggcaccctggtgaccgtgag | |
| | | cagcggtggtggcggttccggcggtggcggttccggtggtggcggttccgaaattgtgttgacacagtctc | |
| | | cagccaccctgtctttgtctccaggggaaagagccaccctcacctgcagcgccaccagcagtgttagctac | |
| | | atccactggtatcaacagaggcctggccagagccccaggctcctcatctatagcacctccaacctggccag | |
| | | cggcatcccagccaggttcagtggcagtgggtctgggagcgactacactctcaccatcagcagcctagag | |
| | | cctgaagattttgcagtttattactgtcagcagcgtagcagctcccctttcacctttggcagcggcaccaaagt | |
| | | ggaaattaaaacaacaacccctgcccccagacctcctaccccagcccctacaattgccagccagcctctga | |
| | | gcctgaggcccgaggcttgtagacctgctgctggcggagccgtgcacaccagaggactggatttcgcctg | |
| | | cgacatctacatctgggcgcccttggccgggacttgtggggtccttctcctgtcactggttatcaccctttact | |
| | | gcaaacggggcagaaagaaactcctgtatatattcaaacaaccatttatgagaccagtacaaactactcaag | |
| | | aggaagatggctgtagctgccgatttccagaagaagaagaaggaggatgtgaactgagagtgaagttcag | |
| | | caggagcgcagacgcccccgcgtacaagcagggccagaaccagctctataacgagctcaatctaggac | |
| | | gaagagaggagtacgatgttttggacaagagacgtggccgggaccctgagatggggggaaagccgaga | |
| | | aggaagaaccctcaggaaggcctgttcaatgaactgcagaaagataagatggcggaggccttcagtgag | |
| | | attgggatgaaaggcgagcgccggaggggcaaggggcacgatggccttttccagggtctcagtacagcc | |
| | | accaaggacaccttcgacgcccttcacatgcaggccctgccccctcgc | |
| 20A10N | CD8- | atggccttaccagtgaccgccttgctcctgccgctggccttgctgctccacgccgccaggccgcaggtgca | 92 |
| | CD8- | gctggttgaatctggcggcggacttgtgaagcctggcggatctctgagactgagctgtgccgccagcggct | |
| | 41BB | tcacctttagcacatacgccatgagctggatcagacaggcccctggcaaaggcctggaatgggtggcgtct | |
| | 1XX | attggcagagccggcagcacctactacagcgactctgtgaagggcagattcaccatcagccgggacaac | |
| | | gccaagaacagcctgtacctgcagatgaactccctgagagccgaggacaccgccgtgtactattgtgcca | |
| | | gaggacccatctacaacgactacgacgagttcgcctattggggccagggcacactggtcacagtttctagc | |
| | | ggcggtggcggaagcggaggcggtggctccggtggcggaggcagcgaaattgtgctgacacagagcc | |
| | | ccgccacactgtcactttctccaggcgaaagagccacactgagctgcaagagcagccagagcgtgctgta | |
| | | ctccagcaaccagaagaactacctggcctggtatcagcagaagcccggccaagctcctcggctgctgatc | |
| | | tattgggccagcacaagagagagcggcatccctgccagattttctggcagcggctctggcaccgatttcac | |
| | | cctgaccataagcagcctggaacctgaggacttcgccgtgtattactgccaccagtacctgagcagcctga | |
| | | cctttggcggaggcaccaaggtggaaatcaagcggacaacaacccctgcccccagacctcctaccccag | |
| | | cccctacaattgccagccagcctctgagcctgaggcccgaggcttgtagacctgctgctggcggagccgt | |
| | | gcacaccagaggactggatttcgcctgcgacatctacatctgggcgcccttggccgggacttgtggggtcc | |
| | | ttctcctgtcactggttatcaccctttactgcaaacggggcagaaagaaactcctgtatatattcaaacaaccat | |
| | | ttatgagaccagtacaaactactcaagaggaagatggctgtagctgccgatttccagaagaagaagaagga | |
| | | ggatgtgaactgagagtgaagttcagcaggagcgcagacgcccccgcgtacaagcagggccagaacca | |
| | | gctctataacgagctcaatctaggacgaagagaggagtacgatgttttggacaagagacgtggccgggac | |
| | | cctgagatggggggaaagccgagaaggaagaaccctcaggaaggcctgttcaatgaactgcagaaagat | |
| | | aagatggcggaggccttcagtgagattgggatgaaaggcgagcgccggaggggcaaggggcacgatg | |
| | | gccttttccagggtctcagtacagccaccaaggacaccttcgacgcccttcacatgcaggccctgccccctc | |
| | | gc | |
| C2 | CD28- | atggccttaccagtgaccgccttgctcctgccgctggccttgctgctccacgccgccaggccggaggtgca | 93 |
| | CD28- | gctggtggagtctgggggaggcctggtcaagcctggggggtccctgagactctcctgtgcagcctctgga | |
| | CD28 | ttcaccttcagtggctatgccatgagctgggtccgccaggctccagggaaggggctggagtgggtctcaac | |
| | 1XX | cattagtagtggcggaacctacatatactaccccgactcagtgaagggccgattcaccatctccagagaca | |
| | | acgccaagaactcactgtatctgcaaatgaacagcctgagagccgaggacacggccgtgtattactgtgcg | |
| | | agacttgggggggataattactacgaatacttcgatgtctggggcaaagggaccacggtcaccgtctcctc | |
| | | cggcggtggcggatccggcggtggcggatccggcggtggcggatccgacattgtgctgacccagtctcc | |
| | | agcctccttggccgtgtctccaggacagagggccaccatcacctgcagagccagtaagagtgtcagtacc | |
| | | agcggatactcctacatgcactggtatcagcagaaaccaggacaacctcctaaactcctgatttacctggca | |
| | | tccaatctggagagcggggtcccagccaggttcagcggcagtgggtctgggaccgatttcaccctcacaat | |
| | | taatcctgtggaagctaatgatactgcaaattattactgtcagcacagtagggagctgcctttcacattcggcg | |
| | | gagggaccaaggtggagatcaaacgaactaagcacctgtgtccttctccactgttccccggccctagcaag | |
| | | cctttctgggtcctggtggtggtgggcggagtgctggcctgctacagcctgctggtgaccgtggcctttatca | |
| | | tcttctgggtgaggagtaagaggagcaggctcctgcacagtgactacatgaacatgactcctagaagacct | |
| | | gggcctaccagaaagcattaccagccctatgccccaccacgcgacttcgcagcctatcgctccagagtga | |
| | | agttcagcaggagcgcagacgcccccgcgtacaagcagggccagaaccagctctataacgagctcaatc | |
| | | taggacgaagagaggagtacgatgttttggacaagagacgtggccgggaccctgagatggggggaaag | |
| | | ccgagaaggaagaaccctcaggaaggcctgttcaatgaactgcagaaagataagatggcggaggccttc | |
| | | agtgagattgggatgaaaggcgagcgccggaggggcaaggggcacgatggccttttccagggtctcagt | |
| | | acagccaccaaggacaccttcgacgcccttcacatgcaggccctgccccctcgc | |
| E6 | CD28- | atggccttaccagtgaccgccttgctcctgccgctggccttgctgctccacgccgccaggccggaggtgca | 94 |
| | CD28- | gctggtggagtctgggggaggcctggtcaagcctggggggtccctgagactctcctgtgcagcctctgga | |
| | CD28 | ttcaccttcagtaggtatggcatgagctgggtccgccaggctccagggaagaggctggagtgggtctcaac | |
| | 1XX | cattagtggcggaggcacctacatatactacccagactcagtgaagggccgattcaccatctccagagaca | |
| | | acgccaagaacaccctgtatctgcaaatgaacagcctgagagccgaggacacggctgtgtattactgtacc | |
| | | agagataactatggccgcaactatgattatggcatggattattggggccagggcaccctggtgaccgtgag | |
| | | cagcggtggtggcggttccggcggtggcggttccggtggtggcggttccgaaattgtgttgacacagtctc | |
| | | cagccaccctgtctttgtctccaggggaaagagccaccctcacctgcagcgccaccagcagtgttagctac | |
| | | atccactggtatcaacagaggcctggccagagccccaggctcctcatctatagcacctccaacctggccag | |
| | | cggcatcccagccaggttcagtggcagtgggtctgggagcgactacactctcaccatcagcagcctagag | |
| | | cctgaagattttgcagtttattactgtcagcagcgtagcagctcccctttcacctttggcagcggcaccaaagt | |
| | | ggaaattaaaaagcacctgtgtccttctccactgttccccggccctagcaagcctttctgggtcctggtggtg | |
| | | gtgggcggagtgctggcctgctacagcctgctggtgaccgtggcctttatcatcttctgggtgaggagtaag | |
| | | aggagcaggctcctgcacagtgactacatgaacatgactcctagaagacctgggcctaccagaaagcatt | |
| | | accagccctatgccccaccacgcgacttcgcagcctatcgctccagagtgaagttcagcaggagcgcaga | |
| | | cgcccccgcgtacaagcagggccagaaccagctctataacgagctcaatctaggacgaagagaggagta | |
| | | cgatgttttggacaagagacgtggccgggaccctgagatggggggaaagccgagaaggaagaaccctc | |
| | | aggaaggcctgttcaatgaactgcagaaagataagatggcggaggccttcagtgagattgggatgaaagg | |
| | | cgagcgccggaggggcaaggggcacgatggccttttccagggtctcagtacagccaccaaggacacctt | |
| | | cgacgcccttcacatgcaggccctgccccctcgc | |
| 20A10N | CD28- | atggccttaccagtgaccgccttgctcctgccgctggccttgctgctccacgccgccaggccgcaggtgca | 95 |
| | CD28- | gctggttgaatctggcggcggacttgtgaagcctggcggatctctgagactgagctgtgccgccagcggct | |
| | CD28 | tcacctttagcacatacgccatgagctggatcagacaggcccctggcaaaggcctggaatgggtggcgtct | |
| | 1XX | attggcagagccggcagcacctactacagcgactctgtgaagggcagattcaccatcagccgggacaac | |
| | | gccaagaacagcctgtacctgcagatgaactccctgagagccgaggacaccgccgtgtactattgtgcca | |
| | | gaggacccatctacaacgactacgacgagttcgcctattggggccagggcacactggtcacagtttctagc | |
| | | ggcggtggcggaagcggaggcggtggctccggtggcggaggcagcgaaattgtgctgacacagagcc | |
| | | ccgccacactgtcactttctccaggcgaaagagccacactgagctgcaagagcagccagagcgtgctgta | |
| | | ctccagcaaccagaagaactacctggcctggtatcagcagaagcccggccaagctcctcggctgctgatc | |
| | | tattgggccagcacaagagagagcggcatccctgccagattttctggcagcggctctggcaccgatttcac | |
| | | cctgaccataagcagcctggaacctgaggacttcgccgtgtattactgccaccagtacctgagcagcctga | |
| | | cctttggcggaggcaccaaggtggaaatcaagcggaagcacctgtgtccttctccactgttccccggccct | |
| | | agcaagcctttctgggtcctggtggtggtgggcggagtgctggcctgctacagcctgctggtgaccgtggc | |
| | | ctttatcatcttctgggtgaggagtaagaggagcaggctcctgcacagtgactacatgaacatgactcctag | |
| | | aagacctgggcctaccagaaagcattaccagccctatgccccaccacgcgacttcgcagcctatcgctcca | |
| | | gagtgaagttcagcaggagcgcagacgccccgcgtacaagcagggccagaaccagctctataacgagc | |
| | | tcaatctaggacgaagagaggagtacgatgttttggacaagagacgtggccgggaccctgagatggggg | |
| | | gaaagccgagaaggaagaaccctcaggaaggcctgttcaatgaactgcagaaagataagatggcggag | |
| | | gccttcagtgagattgggatgaaaggcgagcgccggaggggcaaggggcacgatggccttttccagggt | |
| | | ctcagtacagccaccaaggacaccttcgacgcccttcacatgcaggccctgccccctcgc | |

| CAR C2 CD8- | atggccttaccagtgaccgccttgctcctgccgctggccttgctgctccacgccgccaggccggaggtgcagctggtg | 96 |
|---|---|---|
| CD8-41BBCD3 | gagtctgggggaggcctggtcaagcctggggggtccctgagactctcctgtgcagcctctggattcaccttcagtggc | |
| 6XFOXP3NFAT | tatgccatgagctgggtccgccaggctccagggaaggggctggagtgggtctcaaccattagtagtggcggaaccta | |
| mCMV | catatactaccccgactcagtgaagggccgattcaccatctccagagacaacgccaagaactcactgtatctgcaaatg | |
| IL18Ralpha | aacagcctgagagccgaggacacggccgtgtattactgtgcgagacttgggggggataattactacgaatacttcgat | |
| | gtctggggcaaagggaccacggtcaccgtctcctccggcggtggcggatccggcggtggcggatccggcggtggc | |
| | ggatccgacattgtgctgacccagtctccagcctccttggccgtgtctccaggacagagggccaccatcacctgcaga | |
| | gccagtaagagtgtcagtaccagcggatactcctacatgcactggtatcagcagaaaccaggacaacctcctaaactc | |
| | ctgatttacctggcatccaatctggagagcggggtcccagccaggttcagcggcagtgggtctgggaccgatttcacc | |
| | ctcacaattaatcctgtggaagctaatgatactgcaaattattactgtcagcacagtagggagctgcctttcacattcggc | |
| | ggagggaccaaggtggagatcaaacgaactacaacaacccctgcccccagacctcctaccccagcccctacaattg | |
| | ccagccagcctctgagcctgaggcccgaggcttgtagacctgctgctggcggagccgtgcacaccagaggactgga | |
| | tttcgcctgcgacatctacatctgggcgcccttggccgggacttgtggggtccttctcctgtcactggttatcaccctttact | |
| | gcaaacggggcagaaagaaactcctgtatatattcaaacaaccatttatgagaccagtacaaactactcaagaggaag | |
| | atggctgtagctgccgatttccagaagaagaagaaggaggatgtgaactgagagtgaagttcagcaggagcgcaga | |
| | cgcccccgcgtacaagcagggccagaaccagctctataacgagctcaatctaggacgaagagaggagtacgatgttt | |
| | tggacaagagacgtggccgggaccctgagatggggggaaagccgagaaggaagaaccctcaggaaggcctgtac | |
| | aatgaactgcagaaagataagatggcggaggcctacagtgagattgggatgaaaggcgagcgccggaggggcaag | |
| | gggcacgatggcctttaccagggtctcagtacagccaccaaggacacctacgacgcccttcacatgcaggccctgcc | |
| | ccctcgctgataagtttaaactgccagaacatttctctggcctaactggccggtaccggcttcattttttccatttactgcag | |
| | aggcttcattttttccatttactgcagaggcttcattttttccatttactgcagaggcttcattttttccatttactgcagaggctt | |
| | cattttttccatttactgcagaggcttcattttttccatttactgcagaactagttaggcgtgtacggtgggaggcctatataa | |
| | gcagagctcgtttagtgaaccgtcagatcgcctggagacgccatccacgctgttttgacctccatagaagacaccggg | |
| | accgatccagcctcgagagacccaatgctagccaccatgaattgtagagaattacccttgaccctttgggtgcttatatct | |
| | gtaagcactgcagaatcttgtacttcacgtccccacattactgtggttgaaggggaacctttctatctgaaacattgctcgt | |
| | gttcacttgcacatgagattgaaacaaccaccaaaagctggtacaaaagcagtggatcacaggaacatgtggagctga | |
| | acccaaggagttccagcagaattgctttgcatgattgtgttttggagttttggccagttgagttgaatgacacaggatctta | |
| | ctttttccaaatgaaaaattatactcagaaatggaaattaaatgtcatcagaagaaataaacacagctgtttcactgaaaga | |
| | caagtaactagtaaaattgtggaagttaaaaaattttttcagataacctgtgaaaacagttactatcaaacactggtcaaca | |
| | gcacatcattgtataagaactgtaaaaagctactactggagaacaataaaaacccaacgataaagaagaacgccgagt | |
| | ttgaagatcaggggtattactcctgcgtgcatttccttcatcataatggaaaactatttaatatcaccaaaaccttcaatataa | |
| | caatagtggaagatcgcagtaatatagttccggttcttcttggaccaaagcttaaccatgttgcagtggaattaggaaaaa | |
| | acgtaaggctcaactgctctgctttgctgaatgaagaggatgtaatttattggatgttcggggaagaaaatggatcggat | |
| | cctaatatacatgaagagaaagaaatgagaattatgactccagaaggcaaatggcatgcttcaaaagtattgagaattga | |
| | aaatattggtgaaagcaatctaaatgttttatataattgcactgtggccagcacgggaggcacagacaccaaaagcttca | |
| | tcttggtgagaaaagcagacatggctgatatcccaggccacgtcttcacaagaggaatgatcatagctgttttgatcttgg | |
| | tggcagtagtgtgcctagtgactgtgtgtgtcatttatagagttgacttggttctattttatagacatttaacgagaagagatg | |
| | aaacattaacagatggaaaaacatatgatgcttttgtgtcttacctaaaagaatgccgacctgaaaatggagaggagcac | |
| | acctttgctgtggagattttgcccagggtgttggagaaacattttgggtataagttatgcatatttgaaagggatgtagtgc | |
| | ctggaggagctgttgttgatgaaatccactcactgatagagaaaagccgaagactaatcattgtcctaagtaaaagttata | |
| | tgtctaatgaggtcaggtatgaacttgaaagtggactccatgaagcattggtggaaagaaaaattaaaataatcttaattg | |
| | aatttacacctgttactgacttcacattcttgccccaatcactaaagcttttgaaatctcacagagttctgaagtggaaggcc | |
| | gataaatctctttcttataactcaaggttctggaagaaccttctttacttaatgcctgcaaaaacagtcaagccaggtagag | |
| | acgaaccggaagtcttgcctgttctttccgagtcttgataa | |
| CAR C2 CD8- | atggccttaccagtgaccgccttgctcctgccgctggccttgctgctccacgccgccaggccggaggtgcagctggtg | 97 |
| CD8-41BBCD3 | gagtctgggggaggcctggtcaagcctggggggtccctgagactctcctgtgcagcctctggattcaccttcagtggc | |
| 3XFOXP3NFAT | tatgccatgagctgggtccgccaggctccagggaaggggctggagtgggtctcaaccattagtagtggcggaaccta | |
| mCMV | catatactaccccgactcagtgaagggccgattcaccatctccagagacaacgccaagaactcactgtatctgcaaatg | |
| IL18Ralpha | aacagcctgagagccgaggacacggccgtgtattactgtgcgagacttgggggggataattactacgaatacttcgat | |
| | gtctggggcaaagggaccacggtcaccgtctcctccggcggtggcggatccggcggtggcggatccggcggtggc | |
| | ggatccgacattgtgctgacccagtctccagcctccttggccgtgtctccaggacagagggccaccatcacctgcaga | |
| | gccagtaagagtgtcagtaccagcggatactcctacatgcactggtatcagcagaaaccaggacaacctcctaaactc | |
| | ctgatttacctggcatccaatctggagagcggggtcccagccaggttcagcggcagtgggtctgggaccgatttcacc | |
| | ctcacaattaatcctgtggaagctaatgatactgcaaattattactgtcagcacagtagggagctgcctttcacattcggc | |
| | ggagggaccaaggtggagatcaaacgaactacaacaacccctgcccccagacctcctaccccagcccctacaattg | |
| | ccagccagcctctgagcctgaggcccgaggcttgtagacctgctgctggcggagccgtgcacaccagaggactgga | |
| | tttcgcctgcgacatctacatctgggcgcccttggccgggacttgtggggtccttctcctgtcactggttatcaccctttact | |
| | gcaaacggggcagaaagaaactcctgtatatattcaaacaaccatttatgagaccagtacaaactactcaagaggaag | |
| | atggctgtagctgccgatttccagaagaagaagaaggaggatgtgaactgagagtgaagttcagcaggagcgcaga | |
| | cgcccccgcgtacaagcagggccagaaccagctctataacgagctcaatctaggacgaagagaggagtacgatgttt | |
| | tggacaagagacgtggccgggaccctgagatggggggaaagccgagaaggaagaaccctcaggaaggcctgtac | |
| | aatgaactgcagaaagataagatggcggaggcctacagtgagattgggatgaaaggcgagcgccggaggggcaag | |
| | gggcacgatggcctttaccagggtctcagtacagccaccaaggacacctacgacgcccttcacatgcaggccctgcc | |
| | ccctcgctgataagtttaaactgccagaacatttctctggcctaactggccggtaccggcttcattttttccatttactgcag | |
| | aggcttcattttttccatttactgcagaggcttcattttttccatttactgcagaactagttaggcgtgtacggtgggaggcct | |
| | atataagcagagctcgtttagtgaaccgtcagatcgcctggagacgccatccacgctgttttgacctccatagaagaca | |
| | ccgggaccgatccagcctcgagagacccaatgctagccaccatgaattgtagagaattacccttgaccctttgggtgc | |
| | ttatatctgtaagcactgcagaatcttgtacttcacgtccccacattactgtggttgaaggggaacctttctatctgaaacatt | |
| | gctcgtgttcacttgcacatgagattgaaacaaccaccaaaagctggtacaaaagcagtggatcacaggaacatgtgg | |
| | agctgaacccaaggagttccagcagaattgctttgcatgattgtgttttggagttttggccagttgagttgaatgacacag | |
| | gatcttactttttccaaatgaaaaattatactcagaaatggaaattaaatgtcatcagaagaaataaacacagctgtttcact | |
| | gaaagacaagtaactagtaaaattgtggaagttaaaaaattttttcagataacctgtgaaaacagttactatcaaacactg | |
| | gtcaacagcacatcattgtataagaactgtaaaaagctactactggagaacaataaaaacccaacgataaagaagaac | |
| | gccgagtttgaagatcaggggtattactcctgcgtgcatttccttcatcataatggaaaactatttaatatcaccaaaacctt | |
| | caatataacaatagtggaagatcgcagtaatatagttccggttcttcttggaccaaagcttaaccatgttgcagtggaatta | |
| | ggaaaaaacgtaaggctcaactgctctgctttgctgaatgaagaggatgtaatttattggatgttcggggaagaaaatgg | |
| | atcggatcctaatatacatgaagagaaagaaatgagaattatgactccagaaggcaaatggcatgcttcaaaagtattga | |
| | gaattgaaaatattggtgaaagcaatctaaatgttttatataattgcactgtggccagcacgggaggcacagacaccaaa | |
| | agcttcatcttggtgagaaaagcagacatggctgatatcccaggccacgtcttcacaagaggaatgatcatagctgtttt | |
| | gatcttggtggcagtagtgtgcctagtgactgtgtgtgtcatttatagagttgacttggttctattttatagacatttaacgag | |
| | aagagatgaaacattaacagatggaaaaacatatgatgcttttgtgtcttacctaaaagaatgccgacctgaaaatggag | |
| | aggagcacacctttgctgtggagattttgcccagggtgttggagaaacattttgggtataagttatgcatatttgaaaggg | |
| | atgtagtgcctggaggagctgttgttgatgaaatccactcactgatagagaaaagccgaagactaatcattgtcctaagt | |
| | aaaagttatatgtctaatgaggtcaggtatgaacttgaaagtggactccatgaagcattggtggaaagaaaaattaaaat | |
| | aatcttaattgaatttacacctgttactgacttcacattcttgccccaatcactaaagcttttgaaatctcacagagttctgaag | |
| | tggaaggccgataaatctctttcttataactcaaggttctggaagaaccttctttacttaatgcctgcaaaaacagtcaagc | |
| | caggtagagacgaaccggaagtcttgcctgttctttccgagtcttgataa | |
| CAR C2 CD8- | atggccttaccagtgaccgccttgctcctgccgctggccttgctgctccacgccgccaggccggaggtgcagctggtg | 98 |
| CD8-41BBCD3 | gagtctgggggaggcctggtcaagcctggggggtccctgagactctcctgtgcagcctctggattcaccttcagtggc | |
| 6XIL2NFAT | tatgccatgagctgggtccgccaggctccagggaaggggctggagtgggtctcaaccattagtagtggcggaaccta | |
| mCMV | catatactaccccgactcagtgaagggccgattcaccatctccagagacaacgccaagaactcactgtatctgcaaatg | |
| IL18Ralpha | aacagcctgagagccgaggacacggccgtgtattactgtgcgagacttgggggggataattactacgaatacttcgat | |
| | gtctggggcaaagggaccacggtcaccgtctcctccggcggtggcggatccggcggtggcggatccggcggtggc | |
| | ggatccgacattgtgctgacccagtctccagcctccttggccgtgtctccaggacagagggccaccatcacctgcaga | |
| | gccagtaagagtgtcagtaccagcggatactcctacatgcactggtatcagcagaaaccaggacaacctcctaaactc | |
| | ctgatttacctggcatccaatctggagagcggggtcccagccaggttcagcggcagtgggtctgggaccgatttcacc | |
| | ctcacaattaatcctgtggaagctaatgatactgcaaattattactgtcagcacagtagggagctgcctttcacattcggc | |
| | ggagggaccaaggtggagatcaaacgaactacaacaacccctgcccccagacctcctaccccagcccctacaattg | |
| | ccagccagcctctgagcctgaggcccgaggcttgtagacctgctgctggcggagccgtgcacaccagaggactgga | |
| | tttcgcctgcgacatctacatctgggcgcccttggccgggacttgtggggtccttctcctgtcactggttatcaccctttact | |
| | gcaaacggggcagaaagaaactcctgtatatattcaaacaaccatttatgagaccagtacaaactactcaagaggaag | |
| | atggctgtagctgccgatttccagaagaagaagaaggaggatgtgaactgagagtgaagttcagcaggagcgcaga | |
| | cgcccccgcgtacaagcagggccagaaccagctctataacgagctcaatctaggacgaagagaggagtacgatgttt | |
| | tggacaagagacgtggccgggaccctgagatggggggaaagccgagaaggaagaaccctcaggaaggcctgtac | |
| | aatgaactgcagaaagataagatggcggaggcctacagtgagattgggatgaaaggcgagcgccggaggggcaag | |
| | gggcacgatggcctttaccagggtctcagtacagccaccaaggacacctacgacgcccttcacatgcaggccctgcc | |
| | ccctcgctgataagtttaaactgccagaacatttctctggcctaactggccggtaccggaggaaaaactgtttcatacag | |
| | aaggcgtggaggaaaaactgtttcatacagaaggcgtggaggaaaaactgtttcatacagaaggcgtggaggaaaaa | |
| | ctgtttcatacagaaggcgtggaggaaaaactgtttcatacagaaggcgtggaggaaaaactgtttcatacagaaggc | |
| | gtactagttaggcgtgtacggtgggaggcctatataagcagagctcgtttagtgaaccgtcagatcgcctggagacgc | |
| | catccacgctgttttgacctccatagaagacaccgggaccgatccagcctcgagagacccaatgctagccaccatga | |
| | attgtagagaattacccttgaccctttgggtgcttatatctgtaagcactgcagaatcttgtacttcacgtccccacattactg | |
| | tggttgaaggggaacctttctatctgaaacattgctcgtgttcacttgcacatgagattgaaacaaccaccaaaagctggt | |
| | acaaaagcagtggatcacaggaacatgtggagctgaacccaaggagttccagcagaattgctttgcatgattgtgttttg | |
| | gagttttggccagttgagttgaatgacacaggatcttactttttccaaatgaaaaattatactcagaaatggaaattaaatgt | |
| | catcagaagaaataaacacagctgtttcactgaaagacaagtaactagtaaaattgtggaagttaaaaaattttttcagata | |
| | acctgtgaaaacagttactatcaaacactggtcaacagcacatcattgtataagaactgtaaaaagctactactggagaa | |
| | caataaaaacccaacgataaagaagaacgccgagtttgaagatcaggggtattactcctgcgtgcatttccttcatcata | |
| | atggaaaactatttaatatcaccaaaaccttcaatataacaatagtggaagatcgcagtaatatagttccggttcttcttgga | |
| | ccaaagcttaaccatgttgcagtggaattaggaaaaaacgtaaggctcaactgctctgctttgctgaatgaagaggatgt | |
| | aatttattggatgttcggggaagaaaatggatcggatcctaatatacatgaagagaaagaaatgagaattatgactccag | |
| | aaggcaaatggcatgcttcaaaagtattgagaattgaaaatattggtgaaagcaatctaaatgttttatataattgcactgtg | |
| | gccagcacgggaggcacagacaccaaaagcttcatcttggtgagaaaagcagacatggctgatatcccaggccacg | |
| | tcttcacaagaggaatgatcatagctgttttgatcttggtggcagtagtgtgcctagtgactgtgtgtgtcatttatagagtt | |
| | gacttggttctattttatagacatttaacgagaagagatgaaacattaacagatggaaaaacatatgatgcttttgtgtctta | |
| | cctaaaagaatgccgacctgaaaatggagaggagcacacctttgctgtggagattttgcccagggtgttggagaaaca | |
| | ttttgggtataagttatgcatatttgaaagggatgtagtgcctggaggagctgttgttgatgaaatccactcactgatagag | |
| | aaaagccgaagactaatcattgtcctaagtaaaagttatatgtctaatgaggtcaggtatgaacttgaaagtggactccat | |
| | gaagcattggtggaaagaaaaattaaaataatcttaattgaatttacacctgttactgacttcacattcttgccccaatcact | |
| | aaagcttttgaaatctcacagagttctgaagtggaaggccgataaatctctttcttataactcaaggttctggaagaacctt | |
| | ctttacttaatgcctgcaaaaacagtcaagccaggtagagacgaaccggaagtcttgcctgttctttccgagtcttgataa | |
| CAR C2 CD8- | atggccttaccagtgaccgccttgctcctgccgctggccttgctgctccacgccgccaggccggaggtgcagctggtg | 99 |
| CD8-41BBCD3 | gagtctgggggaggcctggtcaagcctggggggtccctgagactctcctgtgcagcctctggattcaccttcagtggc | |
| 3XIL2NFAT | tatgccatgagctgggtccgccaggctccagggaaggggctggagtgggtctcaaccattagtagtggcggaaccta | |
| mCMV | catatactaccccgactcagtgaagggccgattcaccatctccagagacaacgccaagaactcactgtatctgcaaatg | |
| IL18Ralpha | aacagcctgagagccgaggacacggccgtgtattactgtgcgagacttgggggggataattactacgaatacttcgat | |
| | gtctggggcaaagggaccacggtcaccgtctcctccggcggtggcggatccggcggtggcggatccggcggtggc | |
| | ggatccgacattgtgctgacccagtctccagcctccttggccgtgtctccaggacagagggccaccatcacctgcaga | |
| | gccagtaagagtgtcagtaccagcggatactcctacatgcactggtatcagcagaaaccaggacaacctcctaaactc | |
| | ctgatttacctggcatccaatctggagagcggggtcccagccaggttcagcggcagtgggtctgggaccgatttcacc | |
| | ctcacaattaatcctgtggaagctaatgatactgcaaattattactgtcagcacagtagggagctgcctttcacattcggc | |
| | ggagggaccaaggtggagatcaaacgaactacaacaacccctgcccccagacctcctaccccagcccctacaattg | |
| | ccagccagcctctgagcctgaggcccgaggcttgtagacctgctgctggcggagccgtgcacaccagaggactgga | |
| | tttcgcctgcgacatctacatctgggcgcccttggccgggacttgtggggtccttctcctgtcactggttatcaccctttact | |
| | gcaaacggggcagaaagaaactcctgtatatattcaaacaaccatttatgagaccagtacaaactactcaagaggaag | |
| | atggctgtagctgccgatttccagaagaagaagaaggaggatgtgaactgagagtgaagttcagcaggagcgcaga | |
| | cgcccccgcgtacaagcagggccagaaccagctctataacgagctcaatctaggacgaagagaggagtacgatgttt | |
| | tggacaagagacgtggccgggaccctgagatggggggaaagccgagaaggaagaaccctcaggaaggcctgtac | |
| | aatgaactgcagaaagataagatggcggaggcctacagtgagattgggatgaaaggcgagcgccggaggggcaag | |
| | gggcacgatggcctttaccagggtctcagtacagccaccaaggacacctacgacgcccttcacatgcaggccctgcc | |
| | ccctcgctgataagtttaaactgccagaacatttctctggcctaactggccggtaccggaggaaaaactgtttcatacag | |
| | aaggcgtggaggaaaaactgtttcatacagaaggcgtggaggaaaaactgtttcatacagaaggcgtactagttaggc | |
| | gtgtacggtgggaggcctatataagcagagctcgtttagtgaaccgtcagatcgcctggagacgccatccacgctgttt | |
| | tgacctccatagaagacaccgggaccgatccagcctcgagagacccaatgctagccaccatgaattgtagagaatta | |
| | cccttgaccctttgggtgcttatatctgtaagcactgcagaatcttgtacttcacgtccccacattactgtggttgaagggg | |
| | aacctttctatctgaaacattgctcgtgttcacttgcacatgagattgaaacaaccaccaaaagctggtacaaaagcagtg | |
| | gatcacaggaacatgtggagctgaacccaaggagttccagcagaattgctttgcatgattgtgttttggagttttggcca | |
| | gttgagttgaatgacacaggatcttactttttccaaatgaaaaattatactcagaaatggaaattaaatgtcatcagaagaa | |
| | ataaacacagctgtttcactgaaagacaagtaactagtaaaattgtggaagttaaaaaattttttcagataacctgtgaaaa | |
| | cagttactatcaaacactggtcaacagcacatcattgtataagaactgtaaaaagctactactggagaacaataaaaacc | |
| | caacgataaagaagaacgccgagtttgaagatcaggggtattactcctgcgtgcatttccttcatcataatggaaaactat | |
| | ttaatatcaccaaaaccttcaatataacaatagtggaagatcgcagtaatatagttccggttcttcttggaccaaagcttaa | |
| | ccatgttgcagtggaattaggaaaaaacgtaaggctcaactgctctgctttgctgaatgaagaggatgtaatttattggat | |
| | gttcggggaagaaaatggatcggatcctaatatacatgaagagaaagaaatgagaattatgactccagaaggcaaatg | |
| | gcatgcttcaaaagtattgagaattgaaaatattggtgaaagcaatctaaatgttttatataattgcactgtggccagcacg | |
| | ggaggcacagacaccaaaagcttcatcttggtgagaaaagcagacatggctgatatcccaggccacgtcttcacaag | |
| | aggaatgatcatagctgttttgatcttggtggcagtagtgtgcctagtgactgtgtgtgtcatttatagagttgacttggttct | |
| | attttatagacatttaacgagaagagatgaaacattaacagatggaaaaacatatgatgcttttgtgtcttacctaaaagaat | |
| | gccgacctgaaaatggagaggagcacacctttgctgtggagattttgcccagggtgttggagaaacattttgggtataa | |
| | gttatgcatatttgaaagggatgtagtgcctggaggagctgttgttgatgaaatccactcactgatagagaaaagccgaa | |
| | gactaatcattgtcctaagtaaaagttatatgtctaatgaggtcaggtatgaacttgaaagtggactccatgaagcattggt | |
| | ggaaagaaaaattaaaataatcttaattgaatttacacctgttactgacttcacattcttgccccaatcactaaagcttttgaa | |
| | atctcacagagttctgaagtggaaggccgataaatctctttcttataactcaaggttctggaagaaccttctttacttaatgc | |
| | ctgcaaaaacagtcaagccaggtagagacgaaccggaagtcttgcctgttctttccgagtcttgataa | |
| CAR C2 CD28- | atggccttaccagtgaccgccttgctcctgccgctggccttgctgctccacgccgccaggccggaggtgcagctggtg | 100 |
| CD28-CD28CD3 | gagtctgggggaggcctggtcaagcctggggggtccctgagactctcctgtgcagcctctggattcaccttcagtggc | |
| 6XFOXP3NFAT | tatgccatgagctgggtccgccaggctccagggaaggggctggagtgggtctcaaccattagtagtggcggaaccta | |
| mCMV | catatactaccccgactcagtgaagggccgattcaccatctccagagacaacgccaagaactcactgtatctgcaaatg | |
| IL18Ralpha | aacagcctgagagccgaggacacggccgtgtattactgtgcgagacttgggggggataattactacgaatacttcgat | |
| | gtctggggcaaagggaccacggtcaccgtctcctccggcggtggcggatccggcggtggcggatccggcggtggc | |
| | ggatccgacattgtgctgacccagtctccagcctccttggccgtgtctccaggacagagggccaccatcacctgcaga | |
| | gccagtaagagtgtcagtaccagcggatactcctacatgcactggtatcagcagaaaccaggacaacctcctaaactc | |
| | ctgatttacctggcatccaatctggagagcggggtcccagccaggttcagcggcagtgggtctgggaccgatttcacc | |
| | ctcacaattaatcctgtggaagctaatgatactgcaaattattactgtcagcacagtagggagctgcctttcacattcggc | |
| | ggagggaccaaggtggagatcaaacgaactaagcacctgtgtccttctccactgttccccggccctagcaagcctttct | |
| | gggtcctggtggtggtgggcggagtgctggcctgctacagcctgctggtgaccgtggcctttatcatcttctgggtgag | |
| | gagtaagaggagcaggctcctgcacagtgactacatgaacatgactcctagaagacctgggcctaccagaaagcatt | |
| | accagccctatgccccaccacgcgacttcgcagcctatcgctccagagtgaagttcagcaggagcgcagacgcccc | |
| | cgcgtacaagcagggccagaaccagctctataacgagctcaatctaggacgaagagaggagtacgatgttttggaca | |
| | agagacgtggccgggaccctgagatggggggaaagccgagaaggaagaaccctcaggaaggcctgtacaatgaa | |
| | ctgcagaaagataagatggcggaggcctacagtgagattgggatgaaaggcgagcgccggaggggcaaggggca | |
| | cgatggcctttaccagggtctcagtacagccaccaaggacacctacgacgcccttcacatgcaggccctgccccctcg | |
| | ctgataagtttaaactgccagaacatttctctggcctaactggccggtaccggcttcattttttccatttactgcagaggctt | |
| | cattttttccatttactgcagaggcttcattttttccatttactgcagaggcttcattttttccatttactgcagaggcttcatttttt | |
| | ccatttactgcagaggcttcattttttccatttactgcagaactagttaggcgtgtacggtgggaggcctatataagcagag | |
| | ctcgtttagtgaaccgtcagatcgcctggagacgccatccacgctgttttgacctccatagaagacaccgggaccgatc | |
| | cagcctcgagagacccaatgctagccaccatgaattgtagagaattacccttgaccctttgggtgcttatatctgtaagc | |
| | actgcagaatcttgtacttcacgtccccacattactgtggttgaaggggaacctttctatctgaaacattgctcgtgttcact | |
| | tgcacatgagattgaaacaaccaccaaaagctggtacaaaagcagtggatcacaggaacatgtggagctgaacccaa | |
| | ggagttccagcagaattgctttgcatgattgtgttttggagttttggccagttgagttgaatgacacaggatcttactttttcc | |
| | aaatgaaaaattatactcagaaatggaaattaaatgtcatcagaagaaataaacacagctgtttcactgaaagacaagta | |
| | actagtaaaattgtggaagttaaaaaattttttcagataacctgtgaaaacagttactatcaaacactggtcaacagcacat | |
| | cattgtataagaactgtaaaaagctactactggagaacaataaaaacccaacgataaagaagaacgccgagtttgaag | |
| | atcaggggtattactcctgcgtgcatttccttcatcataatggaaaactatttaatatcaccaaaaccttcaatataacaata | |
| | gtggaagatcgcagtaatatagttccggttcttcttggaccaaagcttaaccatgttgcagtggaattaggaaaaaacgta | |
| | aggctcaactgctctgctttgctgaatgaagaggatgtaatttattggatgttcggggaagaaaatggatcggatcctaat | |
| | atacatgaagagaaagaaatgagaattatgactccagaaggcaaatggcatgcttcaaaagtattgagaattgaaaatat | |
| | tggtgaaagcaatctaaatgttttatataattgcactgtggccagcacgggaggcacagacaccaaaagcttcatcttgg | |
| | tgagaaaagcagacatggctgatatcccaggccacgtcttcacaagaggaatgatcatagctgttttgatcttggtggca | |
| | gtagtgtgcctagtgactgtgtgtgtcatttatagagttgacttggttctattttatagacatttaacgagaagagatgaaac | |
| | attaacagatggaaaaacatatgatgcttttgtgtcttacctaaaagaatgccgacctgaaaatggagaggagcacacct | |
| | ttgctgtggagattttgcccagggtgttggagaaacattttgggtataagttatgcatatttgaaagggatgtagtgcctgg | |
| | aggagctgttgttgatgaaatccactcactgatagagaaaagccgaagactaatcattgtcctaagtaaaagttatatgtc | |
| | taatgaggtcaggtatgaacttgaaagtggactccatgaagcattggtggaaagaaaaattaaaataatcttaattgaattt | |
| | acacctgttactgacttcacattcttgccccaatcactaaagcttttgaaatctcacagagttctgaagtggaaggccgata | |
| | aatctctttcttataactcaaggttctggaagaaccttctttacttaatgcctgcaaaaacagtcaagccaggtagagacga | |
| | accggaagtcttgcctgttctttccgagtcttgataa | |
| CAR C2 CD28- | atggccttaccagtgaccgccttgctcctgccgctggccttgctgctccacgccgccaggccggaggtgcagctggtg | 101 |
| CD28-CD28CD3 | gagtctgggggaggcctggtcaagcctggggggtccctgagactctcctgtgcagcctctggattcaccttcagtggc | |
| 3XFOXP3NFAT | tatgccatgagctgggtccgccaggctccagggaaggggctggagtgggtctcaaccattagtagtggcggaaccta | |
| mCMV | catatactaccccgactcagtgaagggccgattcaccatctccagagacaacgccaagaactcactgtatctgcaaatg | |
| IL18Ralpha | aacagcctgagagccgaggacacggccgtgtattactgtgcgagacttgggggggataattactacgaatacttcgat | |
| | gtctggggcaaagggaccacggtcaccgtctcctccggcggtggcggatccggcggtggcggatccggcggtggc | |
| | ggatccgacattgtgctgacccagtctccagcctccttggccgtgtctccaggacagagggccaccatcacctgcaga | |
| | gccagtaagagtgtcagtaccagcggatactcctacatgcactggtatcagcagaaaccaggacaacctcctaaactc | |
| | ctgatttacctggcatccaatctggagagcggggtcccagccaggttcagcggcagtgggtctgggaccgatttcacc | |
| | ctcacaattaatcctgtggaagctaatgatactgcaaattattactgtcagcacagtagggagctgcctttcacattcggc | |
| | ggagggaccaaggtggagatcaaacgaactaagcacctgtgtccttctccactgttccccggccctagcaagcctttct | |
| | gggtcctggtggtggtgggcggagtgctggcctgctacagcctgctggtgaccgtggcctttatcatcttctgggtgag | |
| | gagtaagaggagcaggctcctgcacagtgactacatgaacatgactcctagaagacctgggcctaccagaaagcatt | |
| | accagccctatgccccaccacgcgacttcgcagcctatcgctccagagtgaagttcagcaggagcgcagacgcccc | |
| | cgcgtacaagcagggccagaaccagctctataacgagctcaatctaggacgaagagaggagtacgatgttttggaca | |
| | agagacgtggccgggaccctgagatggggggaaagccgagaaggaagaaccctcaggaaggcctgtacaatgaa | |
| | ctgcagaaagataagatggcggaggcctacagtgagattgggatgaaaggcgagcgccggaggggcaaggggca | |
| | cgatggcctttaccagggtctcagtacagccaccaaggacacctacgacgcccttcacatgcaggccctgccccctcg | |
| | ctgataagtttaaactgccagaacatttctctggcctaactggccggtaccggcttcattttttccatttactgcagaggctt | |
| | cattttttccatttactgcagaggcttcattttttccatttactgcagaactagttaggcgtgtacggtgggaggcctatataa | |
| | gcagagctcgtttagtgaaccgtcagatcgcctggagacgccatccacgctgttttgacctccatagaagacaccggg | |
| | accgatccagcctcgagagacccaatgctagccaccatgaattgtagagaattacccttgaccctttgggtgcttatatc | |
| | tgtaagcactgcagaatcttgtacttcacgtccccacattactgtggttgaaggggaacctttctatctgaaacattgctcgt | |
| | gttcacttgcacatgagattgaaacaaccaccaaaagctggtacaaaagcagtggatcacaggaacatgtggagctga | |
| | acccaaggagttccagcagaattgctttgcatgattgtgttttggagttttggccagttgagttgaatgacacaggatctta | |
| | ctttttccaaatgaaaaattatactcagaaatggaaattaaatgtcatcagaagaaataaacacagctgtttcactgaaaga | |
| | caagtaactagtaaaattgtggaagttaaaaaattttttcagataacctgtgaaaacagttactatcaaacactggtcaaca | |
| | gcacatcattgtataagaactgtaaaaagctactactggagaacaataaaaacccaacgataaagaagaacgccgagt | |
| | ttgaagatcaggggtattactcctgcgtgcatttccttcatcataatggaaaactatttaatatcaccaaaaccttcaatataa | |
| | caatagtggaagatcgcagtaatatagttccggttcttcttggaccaaagcttaaccatgttgcagtggaattaggaaaaa | |
| | acgtaaggctcaactgctctgctttgctgaatgaagaggatgtaatttattggatgttcggggaagaaaatggatcggat | |
| | cctaatatacatgaagagaaagaaatgagaattatgactccagaaggcaaatggcatgcttcaaaagtattgagaattga | |
| | aaatattggtgaaagcaatctaaatgttttatataattgcactgtggccagcacgggaggcacagacaccaaaagcttca | |
| | tcttggtgagaaaagcagacatggctgatatcccaggccacgtcttcacaagaggaatgatcatagctgttttgatcttgg | |
| | tggcagtagtgtgcctagtgactgtgtgtgtcatttatagagttgacttggttctattttatagacatttaacgagaagagatg | |
| | aaacattaacagatggaaaaacatatgatgcttttgtgtcttacctaaaagaatgccgacctgaaaatggagaggagcac | |
| | acctttgctgtggagattttgcccagggtgttggagaaacattttgggtataagttatgcatatttgaaagggatgtagtgc | |
| | ctggaggagctgttgttgatgaaatccactcactgatagagaaaagccgaagactaatcattgtcctaagtaaaagttata | |
| | tgtctaatgaggtcaggtatgaacttgaaagtggactccatgaagcattggtggaaagaaaaattaaaataatcttaattg | |
| | aatttacacctgttactgacttcacattcttgccccaatcactaaagcttttgaaatctcacagagttctgaagtggaaggcc | |
| | gataaatctctttcttataactcaaggttctggaagaaccttctttacttaatgcctgcaaaaacagtcaagccaggtagag | |
| | acgaaccggaagtcttgcctgttctttccgagtcttgataa | |
| CAR C2 CD28- | atggccttaccagtgaccgccttgctcctgccgctggccttgctgctccacgccgccaggccggaggtgcagctggtg | 102 |
| CD28-CD28CD3 | gagtctgggggaggcctggtcaagcctggggggtccctgagactctcctgtgcagcctctggattcaccttcagtggc | |
| 6XIL2NFAT | tatgccatgagctgggtccgccaggctccagggaaggggctggagtgggtctcaaccattagtagtggcggaaccta | |
| mCMV | catatactaccccgactcagtgaagggccgattcaccatctccagagacaacgccaagaactcactgtatctgcaaatg | |
| IL18Ralpha | aacagcctgagagccgaggacacggccgtgtattactgtgcgagacttgggggggataattactacgaatacttcgat | |
| | gtctggggcaaagggaccacggtcaccgtctcctccggcggtggcggatccggcggtggcggatccggcggtggc | |
| | ggatccgacattgtgctgacccagtctccagcctccttggccgtgtctccaggacagagggccaccatcacctgcaga | |
| | gccagtaagagtgtcagtaccagcggatactcctacatgcactggtatcagcagaaaccaggacaacctcctaaactc | |
| | ctgatttacctggcatccaatctggagagcggggtcccagccaggttcagcggcagtgggtctgggaccgatttcacc | |
| | ctcacaattaatcctgtggaagctaatgatactgcaaattattactgtcagcacagtagggagctgcctttcacattcggc | |
| | ggagggaccaaggtggagatcaaacgaactaagcacctgtgtccttctccactgttccccggccctagcaagcctttct | |
| | gggtcctggtggtggtgggcggagtgctggcctgctacagcctgctggtgaccgtggcctttatcatcttctgggtgag | |
| | gagtaagaggagcaggctcctgcacagtgactacatgaacatgactcctagaagacctgggcctaccagaaagcatt | |
| | accagccctatgccccaccacgcgacttcgcagcctatcgctccagagtgaagttcagcaggagcgcagacgcccc | |
| | cgcgtacaagcagggccagaaccagctctataacgagctcaatctaggacgaagagaggagtacgatgttttggaca | |
| | agagacgtggccgggaccctgagatggggggaaagccgagaaggaagaaccctcaggaaggcctgtacaatgaa | |
| | ctgcagaaagataagatggcggaggcctacagtgagattgggatgaaaggcgagcgccggaggggcaaggggca | |
| | cgatggcctttaccagggtctcagtacagccaccaaggacacctacgacgcccttcacatgcaggccctgccccctcg | |
| | ctgataagtttaaactgccagaacatttctctggcctaactggccggtaccggaggaaaaactgtttcatacagaaggcg | |
| | tggaggaaaaactgtttcatacagaaggcgtggaggaaaaactgtttcatacagaaggcgtggaggaaaaactgtttc | |
| | atacagaaggcgtggaggaaaaactgtttcatacagaaggcgtggaggaaaaactgtttcatacagaaggcgtactag | |
| | ttaggcgtgtacggtgggaggcctatataagcagagctcgtttagtgaaccgtcagatcgcctggagacgccatccac | |
| | gctgttttgacctccatagaagacaccgggaccgatccagcctcgagagacccaatgctagccaccatgaattgtaga | |
| | gaattacccttgaccctttgggtgcttatatctgtaagcactgcagaatcttgtacttcacgtccccacattactgtggttga | |
| | aggggaacctttctatctgaaacattgctcgtgttcacttgcacatgagattgaaacaaccaccaaaagctggtacaaaa | |
| | gcagtggatcacaggaacatgtggagctgaacccaaggagttccagcagaattgctttgcatgattgtgttttggagtttt | |
| | ggccagttgagttgaatgacacaggatcttactttttccaaatgaaaaattatactcagaaatggaaattaaatgtcatcag | |
| | aagaaataaacacagctgtttcactgaaagacaagtaactagtaaaattgtggaagttaaaaaattttttcagataacctgt | |
| | gaaaacagttactatcaaacactggtcaacagcacatcattgtataagaactgtaaaaagctactactggagaacaataa | |
| | aaacccaacgataaagaagaacgccgagtttgaagatcaggggtattactcctgcgtgcatttccttcatcataatggaa | |
| | aactatttaatatcaccaaaaccttcaatataacaatagtggaagatcgcagtaatatagttccggttcttcttggaccaaa | |
| | gcttaaccatgttgcagtggaattaggaaaaaacgtaaggctcaactgctctgctttgctgaatgaagaggatgtaattta | |
| | ttggatgttcggggaagaaaatggatcggatcctaatatacatgaagagaaagaaatgagaattatgactccagaaggc | |
| | aaatggcatgcttcaaaagtattgagaattgaaaatattggtgaaagcaatctaaatgttttatataattgcactgtggccag | |
| | cacgggaggcacagacaccaaaagcttcatcttggtgagaaaagcagacatggctgatatcccaggccacgtcttca | |
| | caagaggaatgatcatagctgttttgatcttggtggcagtagtgtgcctagtgactgtgtgtgtcatttatagagttgacttg | |
| | gttctattttatagacatttaacgagaagagatgaaacattaacagatggaaaaacatatgatgcttttgtgtcttacctaaa | |
| | agaatgccgacctgaaaatggagaggagcacacctttgctgtggagattttgcccagggtgttggagaaacattttggg | |
| | tataagttatgcatatttgaaagggatgtagtgcctggaggagctgttgttgatgaaatccactcactgatagagaaaagc | |
| | cgaagactaatcattgtcctaagtaaaagttatatgtctaatgaggtcaggtatgaacttgaaagtggactccatgaagca | |
| | ttggtggaaagaaaaattaaaataatcttaattgaatttacacctgttactgacttcacattcttgccccaatcactaaagcttt | |
| | tgaaatctcacagagttctgaagtggaaggccgataaatctctttcttataactcaaggttctggaagaaccttctttactta | |
| | atgcctgcaaaaacagtcaagccaggtagagacgaaccggaagtcttgcctgttctttccgagtcttgataa | |
| CAR C2 CD28- | atggccttaccagtgaccgccttgctcctgccgctggccttgctgctccacgccgccaggccggaggtgcagctggtg | 103 |
| CD28-CD28 CD3 | gagtctgggggaggcctggtcaagcctggggggtccctgagactctcctgtgcagcctctggattcaccttcagtggc | |
| 3XIL2NFAT | tatgccatgagctgggtccgccaggctccagggaaggggctggagtgggtctcaaccattagtagtggcggaaccta | |
| mCMV | catatactaccccgactcagtgaagggccgattcaccatctccagagacaacgccaagaactcactgtatctgcaaatg | |
| IL18Ralpha | aacagcctgagagccgaggacacggccgtgtattactgtgcgagacttgggggggataattactacgaatacttcgat | |
| | gtctggggcaaagggaccacggtcaccgtctcctccggcggtggcggatccggcggtggcggatccggcggtggc | |
| | ggatccgacattgtgctgacccagtctccagcctccttggccgtgtctccaggacagagggccaccatcacctgcaga | |
| | gccagtaagagtgtcagtaccagcggatactcctacatgcactggtatcagcagaaaccaggacaacctcctaaactc | |
| | ctgatttacctggcatccaatctggagagcggggtcccagccaggttcagcggcagtgggtctgggaccgatttcacc | |
| | ctcacaattaatcctgtggaagctaatgatactgcaaattattactgtcagcacagtagggagctgcctttcacattcggc | |
| | ggagggaccaaggtggagatcaaacgaactaagcacctgtgtccttctccactgttccccggccctagcaagcctttct | |
| | gggtcctggtggtggtgggcggagtgctggcctgctacagcctgctggtgaccgtggcctttatcatcttctgggtgag | |
| | gagtaagaggagcaggctcctgcacagtgactacatgaacatgactcctagaagacctgggcctaccagaaagcatt | |
| | accagccctatgccccaccacgcgacttcgcagcctatcgctccagagtgaagttcagcaggagcgagacgcccc | |
| | cgcgtacaagcagggccagaaccagctctataacgagctcaatctaggacgaagagaggagtacgatgttttggaca | |
| | agagacgtggccgggaccctgagatggggggaaagccgagaaggaagaaccctcaggaaggcctgtacaatgaa | |
| | ctgcagaaagataagatggcggaggcctacagtgagattgggatgaaaggcgagcgccggaggggcaaggggca | |
| | cgatggcctttaccagggtctcagtacagccaccaaggacacctacgacgcccttcacatgcaggccctgccccctcg | |
| | ctgataagtttaaactgccagaacatttctctggcctaactggccggtaccggaggaaaaactgtttcatacagaaggcg | |
| | tggaggaaaaactgtttcatacagaaggcgtggaggaaaaactgtttcatacagaaggcgtactagttaggcgtgtacg | |
| | gtgggaggcctatataagcagagctcgtttagtgaaccgtcagatcgcctggagacgccatccacgctgttttgacctc | |
| | catagaagacaccgggaccgatccagcctcgagagacccaatgctagccaccatgaattgtagagaattacccttga | |
| | ccctttgggtgcttatatctgtaagcactgcagaatcttgtacttcacgtccccacattactgtggttgaaggggaacctttc | |
| | tatctgaaacattgctcgtgttcacttgcacatgagattgaaacaaccaccaaaagctggtacaaaagcagtggatcaca | |
| | ggaacatgtggagctgaacccaaggagttccagcagaattgctttgcatgattgtgttttggagttttggccagttgagtt | |
| | gaatgacacaggatcttactttttccaaatgaaaaattatactcagaaatggaaattaaatgtcatcagaagaaataaaca | |
| | cagctgtttcactgaaagacaagtaactagtaaaattgtggaagttaaaaaattttttcagataacctgtgaaaacagttact | |
| | atcaaacactggtcaacagcacatcattgtataagaactgtaaaaagctactactggagaacaataaaaacccaacgat | |
| | aaagaagaacgccgagtttgaagatcaggggtattactcctgcgtgcatttccttcatcataatggaaaactatttaatatc | |
| | accaaaaccttcaatataacaatagtggaagatcgcagtaatatagttccggttcttcttggaccaaagcttaaccatgttg | |
| | cagtggaattaggaaaaaacgtaaggctcaactgctctgctttgctgaatgaagaggatgtaatttattggatgttcggg | |
| | gaagaaaatggatcggatcctaatatacatgaagagaaagaaatgagaattatgactccagaaggcaaatggcatgctt | |
| | caaaagtattgagaattgaaaatattggtgaaagcaatctaaatgttttatataattgcactgtggccagcacgggaggca | |
| | cagacaccaaaagcttcatcttggtgagaaaagcagacatggctgatatcccaggccacgtcttcacaagaggaatga | |
| | tcatagctgttttgatcttggtggcagtagtgtgcctagtgactgtgtgtgtcatttatagagttgacttggttctattttataga | |
| | catttaacgagaagagatgaaacattaacagatggaaaaacatatgatgcttttgtgtcttacctaaaagaatgccgacct | |
| | gaaaatggagaggagcacacctttgctgtggagattttgcccagggtgttggagaaacattttgggtataagttatgcat | |
| | atttgaaagggatgtagtgcctggaggagctgttgttgatgaaatccactcactgatagagaaaagccgaagactaatc | |
| | attgtcctaagtaaaagttatatgtctaatgaggtcaggtatgaacttgaaagtggactccatgaagcattggtggaaaga | |
| | aaaattaaaataatcttaattgaatttacacctgttactgacttcacattcttgccccaatcactaaagcttttgaaatctcaca | |
| | gagttctgaagtggaaggccgataaatctctttcttataactcaaggttctggaagaaccttctttacttaatgcctgcaaaa | |
| | acagtcaagccaggtagagacgaaccggaagtcttgcctgttctttccgagtcttgataa | |
| CAR C2 CD28- | atggccttaccagtgaccgccttgctcctgccgctggccttgctgctccacgccgccaggccggaggtgcagctggtg | 104 |
| CD28-CD281XX | gagtctgggggaggcctggtcaagcctggggggtccctgagactctcctgtgcagcctctggattcaccttcagtggc | |
| 6XFOXP3NFAT | tatgccatgagctgggtccgccaggctccagggaaggggctggagtgggtctcaaccattagtagtggcggaaccta | |
| mCMV | catatactaccccgactcagtgaagggccgattcaccatctccagagacaacgccaagaactcactgtatctgcaaatg | |
| IL18Ralpha | aacagcctgagagccgaggacacggccgtgtattactgtgcgagacttgggggggataattactacgaatacttcgat | |
| | gtctggggcaaagggaccacggtcaccgtctcctccggcggtggcggatccggcggtggcggatccggcggtggc | |
| | ggatccgacattgtgctgacccagtctccagcctccttggccgtgtctccaggacagagggccaccatcacctgcaga | |
| | gccagtaagagtgtcagtaccagcggatactcctacatgcactggtatcagcagaaaccaggacaacctcctaaactc | |
| | ctgatttacctggcatccaatctggagagcggggtcccagccaggttcagcggcagtgggtctgggaccgatttcacc | |
| | ctcacaattaatcctgtggaagctaatgatactgcaaattattactgtcagcacagtagggagctgcctttcacattcggc | |
| | ggagggaccaaggtggagatcaaacgaactaagcacctgtgtccttctccactgttccccggccctagcaagcctttct | |
| | gggtcctggtggtggtgggcggagtgctggcctgctacagcctgctggtgaccgtggcctttatcatcttctgggtgag | |
| | gagtaagaggagcaggctcctgcacagtgactacatgaacatgactcctagaagacctgggcctaccagaaagcatt | |
| | accagccctatgccccaccacgcgacttcgcagcctatcgctccagagtgaagttcagcaggagcgcagacgcccc | |
| | cgcgtacaagcagggccagaaccagctctataacgagctcaatctaggacgaagagaggagtacgatgttttggaca | |
| | agagacgtggccgggaccctgagatggggggaaagccgagaaggaagaaccctcaggaaggcctgttcaatgaa | |
| | ctgcagaaagataagatggcggaggccttcagtgagattgggatgaaaggcgagcgccggaggggcaaggggca | |
| | cgatggccttttccagggtctcagtacagccaccaaggacaccttcgacgcccttcacatgcaggccctgccccctcg | |
| | ctgataagtttaaactgccagaacattactctggcctaactggccggtaccggcttcattttttccatttactgcagaggctt | |
| | cattttttccatttactgcagaggcttcattttttccatttactgcagaggcttcattttttccatttactgcagaggcttcatttttt | |
| | ccatttactgcagaggcttcattttttccatttactgcagaactagttaggcgtgtacggtgggaggcctatataagcagag | |
| | ctcgtttagtgaaccgtcagatcgcctggagacgccatccacgctgttttgacctccatagaagacaccgggaccgatc | |
| | cagcctcgagagacccaatgctagccaccatgaattgtagagaattacccttgaccctttgggtgcttatatctgtaagc | |
| | actgcagaatcttgtacttcacgtccccacattactgtggttgaaggggaacctttctatctgaaacattgctcgtgttcact | |
| | tgcacatgagattgaaacaaccaccaaaagctggtacaaaagcagtggatcacaggaacatgtggagctgaacccaa | |
| | ggagttccagcagaattgctttgcatgattgtgttttggagttttggccagttgagttgaatgacacaggatcttactttttcc | |
| | aaatgaaaaattatactcagaaatggaaattaaatgtcatcagaagaaataaacacagctgtttcactgaaagacaagta | |
| | actagtaaaattgtggaagttaaaaaattttttcagataacctgtgaaaacagttactatcaaacactggtcaacagcacat | |
| | cattgtataagaactgtaaaaagctactactggagaacaataaaaacccaacgataaagaagaacgccgagtttgaag | |
| | atcaggggtattactcctgcgtgcatttccttcatcataatggaaaactatttaatatcaccaaaaccttcaatataacaata | |
| | gtggaagatcgcagtaatatagttccggttcttcttggaccaaagcttaaccatgttgcagtggaattaggaaaaaacgta | |
| | aggctcaactgctctgctttgctgaatgaagaggatgtaatttattggatgttcggggaagaaaatggatcggatcctaat | |
| | atacatgaagagaaagaaatgagaattatgactccagaaggcaaatggcatgcttcaaaagtattgagaattgaaaatat | |
| | tggtgaaagcaatctaaatgttttatataattgcactgtggccagcacgggaggcacagacaccaaaagcttcatcttgg | |
| | tgagaaaagcagacatggctgatatcccaggccacgtcttcacaagaggaatgatcatagctgttttgatcttggtggca | |
| | gtagtgtgcctagtgactgtgtgtgtcatttatagagttgacttggttctattttatagacatttaacgagaagagatgaaac | |
| | attaacagatggaaaaacatatgatgcttttgtgtcttacctaaaagaatgccgacctgaaaatggagaggagcacacct | |
| | ttgctgtggagattttgcccagggtgttggagaaacattttgggtataagttatgcatatttgaaagggatgtagtgcctgg | |
| | aggagctgttgttgatgaaatccactcactgatagagaaaagccgaagactaatcattgtcctaagtaaaagttatatgtc | |
| | taatgaggtcaggtatgaacttgaaagtggactccatgaagcattggtggaaagaaaaattaaaataatcttaattgaattt | |
| | acacctgttactgacttcacattcttgccccaatcactaaagcttttgaaatctcacagagttctgaagtggaaggccgata | |
| | aatctctttcttataactcaaggttctggaagaaccttctttacttaatgcctgcaaaaacagtcaagccaggtagagacga | |
| | accggaagtcttgcctgttctttccgagtcttgataa | |
| CAR C2 CD28- | atggccttaccagtgaccgccttgctcctgccgctggccttgctgctccacgccgccaggccggaggtgcagctggtg | 105 |
| CD28-CD281XX | gagtctgggggaggcctggtcaagcctggggggtccctgagactctcctgtgcagcctctggattcaccttcagtggc | |
| 3XFOXP3NFAT | tatgccatgagctgggtccgccaggctccagggaaggggctggagtgggtctcaaccattagtagtggcggaaccta | |
| mCMV | catatactaccccgactcagtgaagggccgattcaccatctccagagacaacgccaagaactcactgtatctgcaaatg | |
| IL18Ralpha | aacagcctgagagccgaggacacggccgtgtattactgtgcgagacttgggggggataattactacgaatacttcgat | |
| | gtctggggcaaagggaccacggtcaccgtctcctccggcggtggcggatccggcggtggcggatccggcggtggc | |
| | ggatccgacattgtgctgacccagtctccagcctccttggccgtgtctccaggacagagggccaccatcacctgcaga | |
| | gccagtaagagtgtcagtaccagcggatactcctacatgcactggtatcagcagaaaccaggacaacctcctaaactc | |
| | ctgatttacctggcatccaatctggagagcggggtcccagccaggttcagcggcagtgggtctgggaccgatttcacc | |
| | ctcacaattaatcctgtggaagctaatgatactgcaaattattactgtcagcacagtagggagctgcctttcacattcggc | |
| | ggagggaccaaggtggagatcaaacgaactaagcacctgtgtccttctccactgttccccggccctagcaagcctttct | |
| | gggtcctggtggtggtgggcggagtgctggcctgctacagcctgctggtgaccgtggcctttatcatcttctgggtgag | |
| | gagtaagaggagcaggctcctgcacagtgactacatgaacatgactcctagaagacctgggcctaccagaaagcatt | |
| | accagccctatgccccaccacgcgacttcgcagcctatcgctccagagtgaagttcagcaggagcgcagacgcccc | |
| | cgcgtacaagcagggccagaaccagctctataacgagctcaatctaggacgaagagaggagtacgatgttttggaca | |
| | agagacgtggccgggaccctgagatggggggaaagccgagaaggaagaaccctcaggaaggcctgttcaatgaa | |
| | ctgcagaaagataagatggcggaggccttcagtgagattgggatgaaaggcgagcgccggaggggcaaggggca | |
| | cgatggccttttccagggtctcagtacagccaccaaggacaccttcgacgcccttcacatgcaggccctgccccctcg | |
| | ctgataagtttaaactgccagaacattactctggcctaactggccggtaccggcttcattttttccatttactgcagaggctt | |
| | cattttttccatttactgcagaggcttcattttttccatttactgcagaactagttaggcgtgtacggtgggaggcctatataa | |
| | gcagagctcgtttagtgaaccgtcagatcgcctggagacgccatccacgctgttttgacctccatagaagacaccggg | |
| | accgatccagcctcgagagacccaatgctagccaccatgaattgtagagaattacccttgaccctttgggtgcttatatc | |
| | tgtaagcactgcagaatcttgtacttcacgtccccacattactgtggttgaaggggaacctttctatctgaaacattgctcgt | |
| | gttcacttgcacatgagattgaaacaaccaccaaaagctggtacaaaagcagtggatcacaggaacatgtggagctga | |
| | acccaaggagttccagcagaattgctttgcatgattgtgttttggagttttggccagttgagttgaatgacacaggatctta | |
| | ctttttccaaatgaaaaattatactcagaaatggaaattaaatgtcatcagaagaaataaacacagctgtttcactgaaaga | |
| | caagtaactagtaaaattgtggaagttaaaaaattttttcagataacctgtgaaaacagttactatcaaacactggtcaaca | |
| | gcacatcattgtataagaactgtaaaaagctactactggagaacaataaaaacccaacgataaagaagaacgccgagt | |
| | ttgaagatcaggggtattactcctgcgtgcatttccttcatcataatggaaaactatttaatatcaccaaaaccttcaatataa | |
| | caatagtggaagatcgcagtaatatagttccggttcttcttggaccaaagcttaaccatgttgcagtggaattaggaaaaa | |
| | acgtaaggctcaactgctctgctttgctgaatgaagaggatgtaatttattggatgttcggggaagaaaatggatcggat | |
| | cctaatatacatgaagagaaagaaatgagaattatgactccagaaggcaaatggcatgcttcaaaagtattgagaattga | |
| | aaatattggtgaaagcaatctaaatgttttatataattgcactgtggccagcacgggaggcacagacaccaaaagcttca | |
| | tcttggtgagaaaagcagacatggctgatatcccaggccacgtcttcacaagaggaatgatcatagctgttttgatcttgg | |
| | tggcagtagtgtgcctagtgactgtgtgtgtcatttatagagttgacttggttctattttatagacatttaacgagaagagatg | |
| | aaacattaacagatggaaaaacatatgatgcttttgtgtcttacctaaaagaatgccgacctgaaaatggagaggagcac | |
| | acctttgctgtggagattttgcccagggtgttggagaaacattttgggtataagttatgcatatttgaaagggatgtagtgc | |
| | ctggaggagctgttgttgatgaaatccactcactgatagagaaaagccgaagactaatcattgtcctaagtaaaagttata | |
| | tgtctaatgaggtcaggtatgaacttgaaagtggactccatgaagcattggtggaaagaaaaattaaaataatcttaattg | |
| | aatttacacctgttactgacttcacattcttgccccaatcactaaagcttttgaaatctcacagagttctgaagtggaaggcc | |
| | gataaatctctttcttataactcaaggttctggaagaaccttctttacttaatgcctgcaaaaacagtcaagccaggtagag | |
| | acgaaccggaagtcttgcctgttctttccgagtcttgataa | |
| CAR C2 CD28- | atggccttaccagtgaccgccttgctcctgccgctggccttgctgctccacgccgccaggccggaggtgcagctggtg | 106 |
| CD28-CD281XX | gagtctgggggaggcctggtcaagcctggggggtccctgagactctcctgtgcagcctctggattcaccttcagtggc | |
| 6XIL2NFAT | tatgccatgagctgggtccgccaggctccagggaaggggctggagtgggtctcaaccattagtagtggcggaaccta | |
| mCMV | catatactaccccgactcagtgaagggccgattcaccatctccagagacaacgccaagaactcactgtatctgcaaatg | |
| IL18Ralpha | aacagcctgagagccgaggacacggccgtgtattactgtgcgagacttgggggggataattactacgaatacttcgat | |
| | gtctggggcaaagggaccacggtcaccgtctcctccggcggtggcggatccggcggtggcggatccggcggtggc | |
| | ggatccgacattgtgctgacccagtctccagcctccttggccgtgtctccaggacagagggccaccatcacctgcaga | |
| | gccagtaagagtgtcagtaccagcggatactcctacatgcactggtatcagcagaaaccaggacaacctcctaaactc | |
| | ctgatttacctggcatccaatctggagagcggggtcccagccaggttcagcggcagtgggtctgggaccgatttcacc | |
| | ctcacaattaatcctgtggaagctaatgatactgcaaattattactgtcagcacagtagggagctgcctttcacattcggc | |
| | ggagggaccaaggtggagatcaaacgaactaagcacctgtgtccttctccactgttccccggccctagcaagcctttct | |
| | gggtcctggtggtggtgggcggagtgctggcctgctacagcctgctggtgaccgtggcctttatcatcttctgggtgag | |
| | gagtaagaggagcaggctcctgcacagtgactacatgaacatgactcctagaagacctgggcctaccagaaagcatt | |
| | accagccctatgccccaccacgcgacttcgcagcctatcgctccagagtgaagttcagcaggagcgcagacgcccc | |
| | cgcgtacaagcagggccagaaccagctctataacgagctcaatctaggacgaagagaggagtacgatgttttggaca | |
| | agagacgtggccgggaccctgagatggggggaaagccgagaaggaagaaccctcaggaaggcctgttcaatgaa | |
| | ctgcagaaagataagatggcggaggccttcagtgagattgggatgaaaggcgagcgccggaggggcaaggggca | |
| | cgatggccttttccagggtctcagtacagccaccaaggacaccttcgacgcccttcacatgcaggccctgccccctcg | |
| | ctgataagtttaaactgccagaacattactctggcctaactggccggtaccggaggaaaaactgtttcatacagaaggc | |
| | gtggaggaaaaactgtttcatacagaaggcgtggaggaaaaactgtttcatacagaaggcgtggaggaaaaactgttt | |
| | catacagaaggcgtggaggaaaaactgtttcatacagaaggcgtggaggaaaaactgtttcatacagaaggcgtacta | |
| | gttaggcgtgtacggtgggaggcctatataagcagagctcgtttagtgaaccgtcagatcgcctggagacgccatcca | |
| | cgctgttttgacctccatagaagacaccgggaccgatccagcctcgagagacccaatgctagccaccatgaattgtag | |
| | agaattacccttgaccctttgggtgcttatatctgtaagcactgcagaatcttgtacttcacgtccccacattactgtggttg | |
| | aaggggaacctttctatctgaaacattgctcgtgttcacttgcacatgagattgaaacaaccaccaaaagctggtacaaa | |
| | agcagtggatcacaggaacatgtggagctgaacccaaggagttccagcagaattgctttgcatgattgtgttttggagtt | |
| | ttggccagttgagttgaatgacacaggatcttactttttccaaatgaaaaattatactcagaaatggaaattaaatgtcatca | |
| | gaagaaataaacacagctgtttcactgaaagacaagtaactagtaaaattgtggaagttaaaaaattttttcagataacct | |
| | gtgaaaacagttactatcaaacactggtcaacagcacatcattgtataagaactgtaaaaagctactactggagaacaat | |
| | aaaaacccaacgataaagaagaacgccgagtttgaagatcaggggtattactcctgcgtgcatttccttcatcataatgg | |
| | aaaactatttaatatcaccaaaaccttcaatataacaatagtggaagatcgcagtaatatagttccggttcttcttggaccaa | |
| | agcttaaccatgttgcagtggaattaggaaaaaacgtaaggctcaactgctctgctttgctgaatgaagaggatgtaattt | |
| | attggatgttcggggaagaaaatggatcggatcctaatatacatgaagagaaagaaatgagaattatgactccagaagg | |
| | caaatggcatgcttcaaaagtattgagaattgaaaatattggtgaaagcaatctaaatgttttatataattgcactgtggcca | |
| | gcacgggaggcacagacaccaaaagcttcatcttggtgagaaaagcagacatggctgatatcccaggccacgtcttc | |
| | acaagaggaatgatcatagctgttttgatcttggtggcagtagtgtgcctagtgactgtgtgtgtcatttatagagttgactt | |
| | ggttctattttatagacatttaacgagaagagatgaaacattaacagatggaaaaacatatgatgcttttgtgtcttacctaa | |
| | aagaatgccgacctgaaaatggagaggagcacacctttgctgtggagattttgcccagggtgttggagaaacattttgg | |
| | gtataagttatgcatatttgaaagggatgtagtgcctggaggagctgttgttgatgaaatccactcactgatagagaaaag | |
| | ccgaagactaatcattgtcctaagtaaaagttatatgtctaatgaggtcaggtatgaacttgaaagtggactccatgaagc | |
| | attggtggaaagaaaaattaaaataatcttaattgaatttacacctgttactgacttcacattcttgccccaatcactaaagct | |
| | tttgaaatctcacagagttctgaagtggaaggccgataaatctctttcttataactcaaggttctggaagaaccttctttactt | |
| | aatgcctgcaaaaacagtcaagccaggtagagacgaaccggaagtcttgcctgttctttccgagtcttgataa | |
| CAR C2 CD28- | atggccttaccagtgaccgccttgctcctgccgctggccttgctgctccacgccgccaggccggaggtgcagctggtg | 107 |
| CD28-CD281XX | gagtctgggggaggcctggtcaagcctggggggtccctgagactctcctgtgcagcctctggattcaccttcagtggc | |
| 3XIL2NFAT | tatgccatgagctgggtccgccaggctccagggaaggggctggagtgggtctcaaccattagtagtggcggaaccta | |
| mCMV | catatactaccccgactcagtgaagggccgattcaccatctccagagacaacgccaagaactcactgtatctgcaaatg | |
| IL18Ralpha | aacagcctgagagccgaggacacggccgtgtattactgtgcgagacttgggggggataattactacgaatacttcgat | |
| | gtctggggcaaagggaccacggtcaccgtctcctccggcggtggcggatccggcggtggcggatccggcggtggc | |
| | ggatccgacattgtgctgacccagtctccagcctccttggccgtgtctccaggacagagggccaccatcacctgcaga | |
| | gccagtaagagtgtcagtaccagcggatactcctacatgcactggtatcagcagaaaccaggacaacctcctaaactc | |
| | ctgatttacctggcatccaatctggagagcggggtcccagccaggttcagcggcagtgggtctgggaccgatttcacc | |
| | ctcacaattaatcctgtggaagctaatgatactgcaaattattactgtcagcacagtagggagctgcctttcacattcggc | |
| | ggagggaccaaggtggagatcaaacgaactaagcacctgtgtccttctccactgttccccggccctagcaagcctttct | |
| | gggtcctggtggtggtgggcggagtgctggcctgctacagcctgctggtgaccgtggcctttatcatcttctgggtgag | |
| | gagtaagaggagcaggctcctgcacagtgactacatgaacatgactcctagaagacctgggcctaccagaaagcatt | |
| | accagccctatgccccaccacgcgacttcgcagcctatcgctccagagtgaagttcagcaggagcgcagacgcccc | |
| | cgcgtacaagcagggccagaaccagctctataacgagctcaatctaggacgaagagaggagtacgatgttttggaca | |
| | agagacgtggccgggaccctgagatggggggaaagccgagaaggaagaaccctcaggaaggcctgttcaatgaa | |
| | ctgcagaaagataagatggcggaggccttcagtgagattgggatgaaaggcgagcgccggaggggcaaggggca | |
| | cgatggccttttccagggtctcagtacagccaccaaggacaccttcgacgcccttcacatgcaggccctgccccctcg | |
| | ctgataagtttaaactgccagaacattactctggcctaactggccggtaccggaggaaaaactgtttcatacagaaggc | |
| | gtggaggaaaaactgtttcatacagaaggcgtggaggaaaaactgtttcatacagaaggcgtactagttaggcgtgtac | |
| | ggtgggaggcctatataagcagagctcgtttagtgaaccgtcagatcgcctggagacgccatccacgctgttttgacct | |
| | ccatagaagacaccgggaccgatccagcctcgagagacccaatgctagccaccatgaattgtagagaattacccttg | |
| | accctttgggtgcttatatctgtaagcactgcagaatcttgtacttcacgtccccacattactgtggttgaaggggaaccttt | |
| | ctatctgaaacattgctcgtgttcacttgcacatgagattgaaacaaccaccaaaagctggtacaaaagcagtggatcac | |
| | aggaacatgtggagctgaacccaaggagttccagcagaattgctttgcatgattgtgttttggagttttggccagttgagt | |
| | tgaatgacacaggatcttactttttccaaatgaaaaattatactcagaaatggaaattaaatgtcatcagaagaaataaaca | |
| | cagctgtttcactgaaagacaagtaactagtaaaattgtggaagttaaaaaattttttcagataacctgtgaaaacagttact | |
| | atcaaacactggtcaacagcacatcattgtataagaactgtaaaaagctactactggagaacaataaaaacccaacgat | |
| | aaagaagaacgccgagtttgaagatcaggggtattactcctgcgtgcatttccttcatcataatggaaaactatttaatatc | |
| | accaaaaccttcaatataacaatagtggaagatcgcagtaatatagttccggttcttcttggaccaaagcttaaccatgttg | |
| | cagtggaattaggaaaaaacgtaaggctcaactgctctgctttgctgaatgaagaggatgtaatttattggatgttcggg | |
| | gaagaaaatggatcggatcctaatatacatgaagagaaagaaatgagaattatgactccagaaggcaaatggcatgctt | |
| | caaaagtattgagaattgaaaatattggtgaaagcaatctaaatgttttatataattgcactgtggccagcacgggaggca | |
| | cagacaccaaaagcttcatcttggtgagaaaagcagacatggctgatatcccaggccacgtcttcacaagaggaatga | |
| | tcatagctgttttgatcttggtggcagtagtgtgcctagtgactgtgtgtgtcatttatagagttgacttggttctattttataga | |
| | catttaacgagaagagatgaaacattaacagatggaaaaacatatgatgcttttgtgtcttacctaaaagaatgccgacct | |
| | gaaaatggagaggagcacacctttgctgtggagattttgcccagggtgttggagaaacattttgggtataagttatgcat | |
| | atttgaaagggatgtagtgcctggaggagctgttgttgatgaaatccactcactgatagagaaaagccgaagactaatc | |
| | attgtcctaagtaaaagttatatgtctaatgaggtcaggtatgaacttgaaagtggactccatgaagcattggtggaaaga | |
| | aaaattaaaataatcttaattgaatttacacctgttactgacttcacattcttgccccaatcactaaagcttttgaaatctcaca | |
| | gagttctgaagtggaaggccgataaatctctttcttataactcaaggttctggaagaaccttctttacttaatgcctgcaaaa | |
| | acagtcaagccaggtagagacgaaccggaagtcttgcctgttctttccgagtcttgataa | |
| CAR E6 CD8- | atggccttaccagtgaccgccttgctcctgccgctggccttgctgctccacgccgccaggccggaggtgcagctggtg | 108 |
| CD8-41BBCD3 | gagtctgggggaggcctggtcaagcctggggggtccctgagactctcctgtgcagcctctggattcaccttcagtagg | |
| 6XFOXP3NFAT | tatggcatgagctgggtccgccaggctccagggaagaggctggagtgggtctcaaccattagtggcggaggcacct | |
| mCMV | acatatactacccagactcagtgaagggccgattcaccatctccagagacaacgccaagaacaccctgtatctgcaaat | |
| IL18Ralpha | gaacagcctgagagccgaggacacggctgtgtattactgtaccagagataactatggccgcaactatgattatggcatg | |
| | gattattggggccagggcaccctggtgaccgtgagcagcggtggtggcggttccggcggtggcggttccggtggtg | |
| | gcggttccgaaattgtgttgacacagtctccagccaccctgtctttgtctccaggggaaagagccaccctcacctgcag | |
| | cgccaccagcagtgttagctacatccactggtatcaacagaggcctggccagagccccaggctcctcatctatagcac | |
| | ctccaacctggccagcggcatcccagccaggttcagtggcagtgggtctgggagcgactacactctcaccatcagca | |
| | gcctagagcctgaagattttgcagtttattactgtcagcagcgtagcagctcccctttcacctttggcagcggcaccaaa | |
| | gtggaaattaaaacaacaacccctgcccccagacctcctaccccagcccctacaattgccagccagcctctgagcctg | |
| | aggcccgaggcttgtagacctgctgctggcggagccgtgcacaccagaggactggatttcgcctgcgacatctacatc | |
| | tgggcgcccttggccgggacttgtggggtccttctcctgtcactggttatcaccctttactgcaaacggggcagaaaga | |
| | aactcctgtatatattcaaacaaccatttatgagaccagtacaaactactcaagaggaagatggctgtagctgccgatttc | |
| | cagaagaagaagaaggaggatgtgaactgagagtgaagttcagcaggagcgcagacgcccccgcgtacaagcag | |
| | ggccagaaccagctctataacgagctcaatctaggacgaagagaggagtacgatgttttggacaagagacgtggccg | |
| | ggaccctgagatggggggaaagccgagaaggaagaaccctcaggaaggcctgtacaatgaactgcagaaagataa | |
| | gatggcggaggcctacagtgagattgggatgaaaggcgagcgccggaggggcaaggggcacgatggcctttacca | |
| | gggtctcagtacagccaccaaggacacctacgacgcccttcacatgcaggccctgccccctcgctgataagtttaaact | |
| | gccagaacatttctctggcctaactggccggtaccggcttcattttttccatttactgcagaggcttcattttttccatttactg | |
| | cagaggcttcattttttccatttactgcagaggcttcattttttccatttactgcagaggcttcattttttccatttactgcagag | |
| | gcttcattttttccatttactgcagaactagttaggcgtgtacggtgggaggcctatataagcagagctcgtttagtgaacc | |
| | gtcagatcgcctggagacgccatccacgctgttttgacctccatagaagacaccgggaccgatccagcctcgagaga | |
| | cccaatgctagccaccatgaattgtagagaattacccttgaccctttgggtgcttatatctgtaagcactgcagaatcttgt | |
| | acttcacgtccccacattactgtggttgaaggggaacctttctatctgaaacattgctcgtgttcacttgcacatgagattga | |
| | aacaaccaccaaaagctggtacaaaagcagtggatcacaggaacatgtggagctgaacccaaggagttccagcaga | |
| | attgctttgcatgattgtgttttggagttttggccagttgagttgaatgacacaggatcttactttttccaaatgaaaaattata | |
| | ctcagaaatggaaattaaatgtcatcagaagaaataaacacagctgtttcactgaaagacaagtaactagtaaaattgtg | |
| | gaagttaaaaaattttttcagataacctgtgaaaacagttactatcaaacactggtcaacagcacatcattgtataagaact | |
| | gtaaaaagctactactggagaacaataaaaacccaacgataaagaagaacgccgagtttgaagatcaggggtattact | |
| | cctgcgtgcatttccttcatcataatggaaaactatttaatatcaccaaaaccttcaatataacaatagtggaagatcgcagt | |
| | aatatagttccggttcttcttggaccaaagcttaaccatgttgcagtggaattaggaaaaaacgtaaggctcaactgctct | |
| | gctttgctgaatgaagaggatgtaatttattggatgttcggggaagaaaatggatcggatcctaatatacatgaagagaa | |
| | agaaatgagaattatgactccagaaggcaaatggcatgcttcaaaagtattgagaattgaaaatattggtgaaagcaatc | |
| | taaatgttttatataattgcactgtggccagcacgggaggcacagacaccaaaagcttcatcttggtgagaaaagcaga | |
| | catggctgatatcccaggccacgtcttcacaagaggaatgatcatagctgttttgatcttggtggcagtagtgtgcctagt | |
| | gactgtgtgtgtcatttatagagttgacttggttctattttatagacatttaacgagaagagatgaaacattaacagatggaa | |
| | aaacatatgatgcttttgtgtcttacctaaaagaatgccgacctgaaaatggagaggagcacacctttgctgtggagatttt | |
| | gcccagggtgttggagaaacattttgggtataagttatgcatatttgaaagggatgtagtgcctggaggagctgttgttga | |
| | tgaaatccactcactgatagagaaaagccgaagactaatcattgtcctaagtaaaagttatatgtctaatgaggtcaggta | |
| | tgaacttgaaagtggactccatgaagcattggtggaaagaaaaattaaaataatcttaattgaatttacacctgttactgac | |
| | ttcacattcttgccccaatcactaaagcttttgaaatctcacagagttctgaagtggaaggccgataaatctctttcttataac | |
| | tcaaggttctggaagaaccttctttacttaatgcctgcaaaaacagtcaagccaggtagagacgaaccggaagtcttgc | |
| | ctgttctttccgagtctggaagcggtgagggaagaggtagcctgctcacgtgcggtgatgtggaggagaaccctggg | |
| | cctatgctctgtttgggctggatatttctttggcttgttgcaggagagcgaattaaaggatttaatatttcaggttgttccaca | |
| | aaaaaactcctttggacatattctacaaggagtgaagaggaatttgtcttattttgtgatttaccagagccacagaaatcac | |
| | atttctgccacagaaatcgactctcaccaaaacaagtccctgagcacctgcccttcatgggtagtaacgacctatctgat | |
| | gtccaatggtaccaacaaccttcgaatggagatccattagaggacattaggaaaagctatcctcacatcattcaggaca | |
| | aatgtacccttcactttttgaccccaggggtgaataattctgggtcatatatttgtagacccaagatgattaagagcccctat | |
| | gatgtagcctgttgtgtcaagatgattttagaagttaagccccagacaaatgcatcctgtgagtattccgcatcacataag | |
| | caagacctacttcttgggagcactggctctatttcttgccccagtctcagctgccaaagtgatgcacaaagtccagcggt | |
| | aacctggtacaagaatggaaaactcctctctgtggaaaggagcaaccgaatcgtagtggatgaagtttatgactatcac | |
| | cagggcacatatgtatgtgattacactcagtcggatactgtgagttcgtggacagtcagagctgttgttcaagtgagaac | |
| | cattgtgggagacactaaactcaaaccagatattctggatcctgtcgaggacacactggaagtagaacttggaaagcct | |
| | ttaactattagctgcaaagcacgatttggctttgaaagggtctttaaccctgtcataaaatggtacatcaaagattctgacct | |
| | agagtgggaagtctcagtacctgaggcgaaaagtattaaatccactttaaaggatgaaatcattgagcgtaatatcatctt | |
| | ggaaaaagtcactcagcgtgatcttcgcaggaagtttgtttgctttgtccagaactccattggaaacacaacccagtccgt | |
| | ccaactgaaagaaaagagaggagtggtgctcctgtacatcctgcttggcaccatcgggaccctggtggccgtgctgg | |
| | cggcgagtgccctcctctacaggcactggattgaaatagtgctgctgtaccggacctaccagagcaaggatcagacg | |
| | cttggggataaaaaggattttgatgctttcgtatcctatgcaaaatggagctcttttccaagtgaggccacttcatctctgag | |
| | tgaagaacacttggccctgagcctatttcctgatgttttagaaaacaaatatggatatagcctgtgtttgcttgaaagagat | |
| | gtggctccaggaggagtgtatgcagaagacattgtgagcattattaagagaagcagaagaggaatatttatcttgagcc | |
| | ccaactatgtcaatggacccagtatctttgaactacaagcagcagtgaatcttgccttggatgatcaaacactgaaactca | |
| | ttttaattaagttctgttacttccaagagccagagtctctacctcatctcgtgaaaaaagctctcagggttttgcccacagtta | |
| | cttggagaggcttaaaatcagttcctcccaattctaggttctgggccaaaatgcgctaccacatgcctgtgaaaaactctc | |
| | agggattcacgtggaaccagctcagaattacctctaggatttttcagtggaaaggactcagtagaacagaaaccactgg | |
| | gaggagctcccagcctaaggaatggtgataa | |
| CAR E6 CD8- | atggccttaccagtgaccgccttgctcctgccgctggccttgctgctccacgccgccaggccggaggtgcagctggtg | 109 |
| CD8-41BBCD3 | gagtctgggggaggcctggtcaagcctggggggtccctgagactctcctgtgcagcctctggattcaccttcagtagg | |
| 3XFOXP3NFAT | tatggcatgagctgggtccgccaggctccagggaagaggctggagtgggtctcaaccattagtggcggaggcacct | |
| mCMV | acatatactacccagactcagtgaagggccgattcaccatctccagagacaacgccaagaacaccctgtatctgcaaat | |
| IL18Ralpha | gaacagcctgagagccgaggacacggctgtgtattactgtaccagagataactatggccgcaactatgattatggcatg | |
| | gattattggggccagggcaccctggtgaccgtgagcagcggtggtggcggttccggcggtggcggttccggtggtg | |
| | gcggttccgaaattgtgttgacacagtctccagccaccctgtctttgtctccaggggaaagagccaccctcacctgcag | |
| | cgccaccagcagtgttagctacatccactggtatcaacagaggcctggccagagccccaggctcctcatctatagcac | |
| | ctccaacctggccagcggcatcccagccaggttcagtggcagtgggtctgggagcgactacactctcaccatcagca | |
| | gcctagagcctgaagattttgcagtttattactgtcagcagcgtagcagctcccctttcacctttggcagcggcaccaaa | |
| | gtggaaattaaaacaacaacccctgcccccagacctcctaccccagcccctacaattgccagccagcctctgagcctg | |
| | aggcccgaggcttgtagacctgctgctggcggagccgtgcacaccagaggactggatttcgcctgcgacatctacatc | |
| | tgggcgcccttggccgggacttgtggggtccttctcctgtcactggttatcaccctttactgcaaacggggcagaaaga | |
| | aactcctgtatatattcaaacaaccatttatgagaccagtacaaactactcaagaggaagatggctgtagctgccgatttc | |
| | cagaagaagaagaaggaggatgtgaactgagagtgaagttcagcaggagcgcagacgcccccgcgtacaagcag | |
| | ggccagaaccagctctataacgagctcaatctaggacgaagagaggagtacgatgttttggacaagagacgtggccg | |
| | ggaccctgagatggggggaaagccgagaaggaagaaccctcaggaaggcctgtacaatgaactgcagaaagataa | |
| | gatggcggaggcctacagtgagattgggatgaaaggcgagcgccggaggggcaaggggcacgatggcctttacca | |
| | gggtctcagtacagccaccaaggacacctacgacgcccttcacatgcaggccctgccccctcgctgataagtttaaact | |
| | gccagaacatttctctggcctaactggccggtaccggcttcattttttccatttactgcagaggcttcattttttccatttactg | |
| | cagaggcttcattttttccatttactgcagaactagttaggcgtgtacggtgggaggcctatataagcagagctcgtttagt | |
| | gaaccgtcagatcgcctggagacgccatccacgctgttttgacctccatagaagacaccgggaccgatccagcctcg | |
| | agagacccaatgctagccaccatgaattgtagagaattacccttgaccctttgggtgcttatatctgtaagcactgcaga | |
| | atcttgtacttcacgtccccacattactgtggttgaaggggaacctttctatctgaaacattgctcgtgttcacttgcacatg | |
| | agattgaaacaaccaccaaaagctggtacaaaagcagtggatcacaggaacatgtggagctgaacccaaggagttcc | |
| | agcagaattgctttgcatgattgtgttttggagttttggccagttgagttgaatgacacaggatcttactttttccaaatgaaa | |
| | aattatactcagaaatggaaattaaatgtcatcagaagaaataaacacagctgtttcactgaaagacaagtaactagtaaa | |
| | attgtggaagttaaaaaattttttcagataacctgtgaaaacagttactatcaaacactggtcaacagcacatcattgtataa | |
| | gaactgtaaaaagctactactggagaacaataaaaacccaacgataaagaagaacgccgagtttgaagatcaggggt | |
| | attactcctgcgtgcatttccttcatcataatggaaaactatttaatatcaccaaaaccttcaatataacaatagtggaagatc | |
| | gcagtaatatagttccggttcttcttggaccaaagcttaaccatgttgcagtggaattaggaaaaaacgtaaggctcaact | |
| | gctctgctttgctgaatgaagaggatgtaatttattggatgttcggggaagaaaatggatcggatcctaatatacatgaag | |
| | agaaagaaatgagaattatgactccagaaggcaaatggcatgcttcaaaagtattgagaattgaaaatattggtgaaag | |
| | caatctaaatgttttatataattgcactgtggccagcacgggaggcacagacaccaaaagcttcatcttggtgagaaaag | |
| | cagacatggctgatatcccaggccacgtcttcacaagaggaatgatcatagctgttttgatcttggtggcagtagtgtgc | |
| | ctagtgactgtgtgtgtcatttatagagttgacttggttctattttatagacatttaacgagaagagatgaaacattaacagat | |
| | ggaaaaacatatgatgcttttgtgtcttacctaaaagaatgccgacctgaaaatggagaggagcacacctttgctgtgga | |
| | gattttgcccagggtgttggagaaacattttgggtataagttatgcatatttgaaagggatgtagtgcctggaggagctgtt | |
| | gttgatgaaatccactcactgatagagaaaagccgaagactaatcattgtcctaagtaaaagttatatgtctaatgaggtc | |
| | aggtatgaacttgaaagtggactccatgaagcattggtggaaagaaaaattaaaataatcttaattgaatttacacctgtta | |
| | ctgacttcacattcttgccccaatcactaaagcttttgaaatctcacagagttctgaagtggaaggccgataaatctctttct | |
| | tataactcaaggttctggaagaaccttctttacttaatgcctgcaaaaacagtcaagccaggtagagacgaaccggaagt | |
| | cttgcctgttctttccgagtcttgataa | |
| CAR E6 CD8- | atggccttaccagtgaccgccttgctcctgccgctggccttgctgctccacgccgccaggccggaggtgcagctggtg | 110 |
| CD8-41BBCD3 | gagtctgggggaggcctggtcaagcctggggggtccctgagactctcctgtgcagcctctggattcaccttcagtagg | |
| 6XIL2NFAT | tatggcatgagctgggtccgccaggctccagggaagaggctggagtgggtctcaaccattagtggggaggcacct | |
| mCMV | acatatactacccagactcagtgaagggccgattcaccatctccagagacaacgccaagaacaccctgtatctgcaaat | |
| IL18Ralpha | gaacagcctgagagccgaggacacggctgtgtattactgtaccagagataactatggccgcaactatgattatggcatg | |
| | gattattggggccagggcaccctggtgaccgtgagcagcggtggtggcggttccggcggtggcggttccggtggtg | |
| | gcggttccgaaattgtgttgacacagtctccagccaccctgtctttgtctccaggggaaagagccaccctcacctgcag | |
| | cgccaccagcagtgttagctacatccactggtatcaacagaggcctggccagagccccaggctcctcatctatagcac | |
| | ctccaacctggccagcggcatcccagccaggttcagtggcagtgggtctgggagcgactacactctcaccatcagca | |
| | gcctagagcctgaagattttgcagtttattactgtcagcagcgtagcagctcccctttcacctttggcagcggcaccaaa | |
| | gtggaaattaaaacaacaacccctgcccccagacctcctaccccagcccctacaattgccagccagcctctgagcctg | |
| | aggcccgaggcttgtagacctgctgctggcggagccgtgcacaccagaggactggatttcgcctgcgacatctacatc | |
| | tgggcgcccttggccgggacttgtggggtccttctcctgtcactggttatcaccctttactgcaaacggggcagaaaga | |
| | aactcctgtatatattcaaacaaccatttatgagaccagtacaaactactcaagaggaagatggctgtagctgccgatttc | |
| | cagaagaagaagaaggaggatgtgaactgagagtgaagttcagcaggagcgcagacgcccccgcgtacaagcag | |
| | ggccagaaccagctctataacgagctcaatctaggacgaagagaggagtacgatgttttggacaagagacgtggccg | |
| | ggaccctgagatggggggaaagccgagaaggaagaaccctcaggaaggcctgtacaatgaactgcagaaagataa | |
| | gatggcggaggcctacagtgagattgggatgaaaggcgagcgccggaggggcaaggggcacgatggcctttacca | |
| | gggtctcagtacagccaccaaggacacctacgacgcccttcacatgcaggccctgccccctcgctgataagtttaaact | |
| | gccagaacatttctctggcctaactggccggtaccggaggaaaaactgtttcatacagaaggcgtggaggaaaaactg | |
| | tttcatacagaaggcgtggaggaaaaactgtttcatacagaaggcgtggaggaaaaactgtttcatacagaaggcgtg | |
| | gaggaaaaactgtttcatacagaaggcgtggaggaaaaactgtttcatacagaaggcgtactagttaggcgtgtacggt | |
| | gggaggcctatataagcagagctcgtttagtgaaccgtcagatcgcctggagacgccatccacgctgttttgacctcca | |
| | tagaagacaccgggaccgatccagcctcgagagacccaatgctagccaccatgaattgtagagaattacccttgacc | |
| | ctttgggtgcttatatctgtaagcactgcagaatcttgtacttcacgtccccacattactgtggttgaaggggaacctttctat | |
| | ctgaaacattgctcgtgttcacttgcacatgagattgaaacaaccaccaaaagctggtacaaaagcagtggatcacagg | |
| | aacatgtggagctgaacccaaggagttccagcagaattgctttgcatgattgtgttttggagttttggccagttgagttgaa | |
| | tgacacaggatcttactttttccaaatgaaaaattatactcagaaatggaaattaaatgtcatcagaagaaataaacacag | |
| | ctgtttcactgaaagacaagtaactagtaaaattgtggaagttaaaaaattttttcagataacctgtgaaaacagttactatc | |
| | aaacactggtcaacagcacatcattgtataagaactgtaaaaagctactactggagaacaataaaaacccaacgataaa | |
| | gaagaacgccgagtttgaagatcaggggtattactcctgcgtgcatttccttcatcataatggaaaactatttaatatcacc | |
| | aaaaccttcaatataacaatagtggaagatcgcagtaatatagttccggttcttcttggaccaaagcttaaccatgttgcag | |
| | tggaattaggaaaaaacgtaaggctcaactgctctgctttgctgaatgaagaggatgtaatttattggatgttcggggaa | |
| | gaaaatggatcggatcctaatatacatgaagagaaagaaatgagaattatgactccagaaggcaaatggcatgcttcaa | |
| | aagtattgagaattgaaaatattggtgaaagcaatctaaatgttttatataattgcactgtggccagcacgggaggcacag | |
| | acaccaaaagcttcatcttggtgagaaaagcagacatggctgatatcccaggccacgtcttcacaagaggaatgatcat | |
| | agctgttttgatcttggtggcagtagtgtgcctagtgactgtgtgtgtcatttatagagttgacttggttctattttatagacatt | |
| | taacgagaagagatgaaacattaacagatggaaaaacatatgatgcttttgtgtcttacctaaaagaatgccgacctgaa | |
| | aatggagaggagcacacctttgctgtggagattttgcccagggtgttggagaaacattttgggtataagttatgcatattt | |
| | gaaagggatgtagtgcctggaggagctgttgttgatgaaatccactcactgatagagaaaagccgaagactaatcattg | |
| | tcctaagtaaaagttatatgtctaatgaggtcaggtatgaacttgaaagtggactccatgaagcattggtggaaagaaaa | |
| | attaaaataatcttaattgaatttacacctgttactgacttcacattcttgccccaatcactaaagcttttgaaatctcacagag | |
| | ttctgaagtggaaggccgataaatctctttcttataactcaaggttctggaagaaccttctttacttaatgcctgcaaaaaca | |
| | gtcaagccaggtagagacgaaccggaagtcttgcctgttctttccgagtcttgataa | |
| CAR E6 CD8- | atggccttaccagtgaccgccttgctcctgccgctggccttgctgctccacgccgccaggccggaggtgcagctggtg | 111 |
| CD8-41BBCD3 | gagtctgggggaggcctggtcaagcctggggggtccctgagactctcctgtgcagcctctggattcaccttcagtagg | |
| 3XIL2NFAT | tatggcatgagctgggtccgccaggctccagggaagaggctggagtgggtctcaaccattagtggcggaggcacct | |
| mCMV | acatatactacccagactcagtgaagggccgattcaccatctccagagacaacgccaagaacaccctgtatctgcaaat | |
| IL18Ralpha | gaacagcctgagagccgaggacacggctgtgtattactgtaccagagataactatggccgcaactatgattatggcatg | |
| | gattattggggccagggcaccctggtgaccgtgagcagcggtggtggcggttccggcggtggcggttccggtggtg | |
| | gcggttccgaaattgtgttgacacagtctccagccaccctgtctttgtctccaggggaaagagccaccctcacctgcag | |
| | cgccaccagcagtgttagctacatccactggtatcaacagaggcctggccagagccccaggctcctcatctatagcac | |
| | ctccaacctggccagcggcatcccagccaggttcagtggcagtgggtctgggagcgactacactctcaccatcagca | |
| | gcctagagcctgaagattttgcagtttattactgtcagcagcgtagcagctcccctttcacctttggcagcggcaccaaa | |
| | gtggaaattaaaacaacaacccctgcccccagacctcctaccccagcccctacaattgccagccagcctctgagcctg | |
| | aggcccgaggcttgtagacctgctgctggcggagccgtgcacaccagaggactggatttcgcctgcgacatctacatc | |
| | tgggcgcccttggccgggacttgtggggtccttctcctgtcactggttatcaccctttactgcaaacggggcagaaaga | |
| | aactcctgtatatattcaaacaaccatttatgagaccagtacaaactactcaagaggaagatggctgtagctgccgatttc | |
| | cagaagaagaagaaggaggatgtgaactgagagtgaagttcagcaggagcgcagacgcccccgcgtacaagcag | |
| | ggccagaaccagctctataacgagctcaatctaggacgaagagaggagtacgatgttttggacaagagacgtggccg | |
| | ggaccctgagatggggggaaagccgagaaggaagaaccctcaggaaggcctgtacaatgaactgcagaaagataa | |
| | gatggcggaggcctacagtgagattgggatgaaaggcgagcgccggaggggcaaggggcacgatggcctttacca | |
| | gggtctcagtacagccaccaaggacacctacgacgcccttcacatgcaggccctgccccctcgctgataagtttaaact | |
| | gccagaacatttctctggcctaactggccggtaccggaggaaaaactgtttcatacagaaggcgtggaggaaaaactg | |
| | tttcatacagaaggcgtggaggaaaaactgtttcatacagaaggcgtactagttaggcgtgtacggtgggaggcctata | |
| | taagcagagctcgtttagtgaaccgtcagatcgcctggagacgccatccacgctgttttgacctccatagaagacaccg | |
| | ggaccgatccagcctcgagagacccaatgctagccaccatgaattgtagagaattacccttgaccctttgggtgcttata | |
| | tctgtaagcactgcagaatcttgtacttcacgtccccacattactgtggttgaaggggaacctttctatctgaaacattgctc | |
| | gtgttcacttgcacatgagattgaaacaaccaccaaaagctggtacaaaagcagtggatcacaggaacatgtggagct | |
| | gaacccaaggagttccagcagaattgctttgcatgattgtgttttggagttttggccagttgagttgaatgacacaggatct | |
| | tactttttccaaatgaaaaattatactcagaaatggaaattaaatgtcatcagaagaaataaacacagctgtttcactgaaa | |
| | gacaagtaactagtaaaattgtggaagttaaaaaattttttcagataacctgtgaaaacagttactatcaaacactggtcaa | |
| | cagcacatcattgtataagaactgtaaaaagctactactggagaacaataaaaacccaacgataaagaagaacgccga | |
| | gtttgaagatcaggggtattactcctgcgtgcatttccttcatcataatggaaaactatttaatatcaccaaaaccttcaatat | |
| | aacaatagtggaagatcgcagtaatatagttccggttcttcttggaccaaagcttaaccatgttgcagtggaattaggaaa | |
| | aaacgtaaggctcaactgctctgctttgctgaatgaagaggatgtaatttattggatgttcggggaagaaaatggatcgg | |
| | atcctaatatacatgaagagaaagaaatgagaattatgactccagaaggcaaatggcatgcttcaaaagtattgagaatt | |
| | gaaaatattggtgaaagcaatctaaatgttttatataattgcactgtggccagcacgggaggcacagacaccaaaagctt | |
| | catcttggtgagaaaagcagacatggctgatatcccaggccacgtcttcacaagaggaatgatcatagctgttttgatctt | |
| | ggtggcagtagtgtgcctagtgactgtgtgtgtcatttatagagttgacttggttctattttatagacatttaacgagaagag | |
| | atgaaacattaacagatggaaaaacatatgatgcttttgtgtcttacctaaaagaatgccgacctgaaaatggagaggag | |
| | cacacctttgctgtggagattttgcccagggtgttggagaaacattttgggtataagttatgcatatttgaaagggatgtag | |
| | tgcctggaggagctgttgttgatgaaatccactcactgatagagaaaagccgaagactaatcattgtcctaagtaaaagt | |
| | tatatgtctaatgaggtcaggtatgaacttgaaagtggactccatgaagcattggtggaaagaaaaattaaaataatctta | |
| | attgaatttacacctgttactgacttcacattcttgccccaatcactaaagcttttgaaatctcacagagttctgaagtggaa | |
| | ggccgataaatctctttcttataactcaaggttctggaagaaccttctttacttaatgcctgcaaaaacagtcaagccaggt | |
| | agagacgaaccggaagtcttgcctgttctttccgagtcttgataa | |
| CAR E6 CD28- | atggccttaccagtgaccgccttgctcctgccgctggccttgctgctccacgccgccaggccggaggtgcagctggtg | 112 |
| CD28-CD28CD3 | gagtctgggggaggcctggtcaagcctggggggtccctgagactctcctgtgcagcctctggattcaccttcagtagg | |
| 6XFOXP3NFAT | tatggcatgagctgggtccgccaggctccagggaagaggctggagtgggtctcaaccattagtggcggaggcacct | |
| mCMV | acatatactacccagactcagtgaagggccgattcaccatctccagagacaacgccaagaacaccctgtatctgcaaat | |
| IL18Ralpha | gaacagcctgagagccgaggacacggctgtgtattactgtaccagagataactatggccgcaactatgattatggcatg | |
| | gattattggggccagggcaccctggtgaccgtgagcagcggtggtggcggttccggcggtggcggttccggtggtg | |
| | gcggttccgaaattgtgttgacacagtctccagccaccctgtctttgtctccaggggaaagagccaccctcacctgcag | |
| | cgccaccagcagtgttagctacatccactggtatcaacagaggcctggccagagccccaggctcctcatctatagcac | |
| | ctccaacctggccagcggcatcccagccaggttcagtggcagtgggtctgggagcgactacactctcaccatcagca | |
| | gcctagagcctgaagattttgcagtttattactgtcagcagcgtagcagctcccctttcacctttggcagcggcaccaaa | |
| | gtggaaattaaaaagcacctgtgtccttctccactgttccccggccctagcaagcctttctgggtcctggtggtggtggg | |
| | cggagtgctggcctgctacagcctgctggtgaccgtggcctttatcatcttctgggtgaggagtaagaggagcaggct | |
| | cctgcacagtgactacatgaacatgactcctagaagacctgggcctaccagaaagcattaccagccctatgccccacc | |
| | acgcgacttcgcagcctatcgctccagagtgaagttcagcaggagcgcagacgcccccgcgtacaagcagggcca | |
| | gaaccagctctataacgagctcaatctaggacgaagagaggagtacgatgttttggacaagagacgtggccgggacc | |
| | ctgagatggggggaaagccgagaaggaagaaccctcaggaaggcctgtacaatgaactgcagaaagataagatgg | |
| | cggaggcctacagtgagattgggatgaaaggcgagcgccggaggggcaaggggcacgatggcctttaccagggtc | |
| | tcagtacagccaccaaggacacctacgacgcccttcacatgcaggccctgccccctcgctgataagtttaaactgcca | |
| | gaacatttctctggcctaactggccggtaccggcttcattttttccatttactgcagaggcttcattttttccatttactgcaga | |
| | ggcttcattttttccatttactgcagaggcttcattttttccatttactgcagaggcttcattttttccatttactgcagaggcttc | |
| | attttttccatttactgcagaactagttaggcgtgtacggtgggaggcctatataagcagagctcgtttagtgaaccgtca | |
| | gatcgcctggagacgccatccacgctgttttgacctccatagaagacaccgggaccgatccagcctcgagagaccca | |
| | atgctagccaccatgaattgtagagaattacccttgaccctttgggtgcttatatctgtaagcactgcagaatcttgtactt | |
| | cacgtccccacattactgtggttgaaggggaacctttctatctgaaacattgctcgtgttcacttgcacatgagattgaaac | |
| | aaccaccaaaagctggtacaaaagcagtggatcacaggaacatgtggagctgaacccaaggagttccagcagaattg | |
| | ctttgcatgattgtgttttggagttttggccagttgagttgaatgacacaggatcttactttttccaaatgaaaaattatactca | |
| | gaaatggaaattaaatgtcatcagaagaaataaacacagctgtttcactgaaagacaagtaactagtaaaattgtggaag | |
| | ttaaaaaattttttcagataacctgtgaaaacagttactatcaaacactggtcaacagcacatcattgtataagaactgtaaa | |
| | aagctactactggagaacaataaaaacccaacgataaagaagaacgccgagtttgaagatcaggggtattactcctgc | |
| | gtgcatttccttcatcataatggaaaactatttaatatcaccaaaaccttcaatataacaatagtggaagatcgcagtaatat | |
| | agttccggttcttcttggaccaaagcttaaccatgttgcagtggaattaggaaaaaacgtaaggctcaactgctctgctttg | |
| | ctgaatgaagaggatgtaatttattggatgttcggggaagaaaatggatcggatcctaatatacatgaagagaaagaaat | |
| | gagaattatgactccagaaggcaaatggcatgcttcaaaagtattgagaattgaaaatattggtgaaagcaatctaaatgt | |
| | tttatataattgcactgtggccagcacgggaggcacagacaccaaaagcttcatcttggtgagaaaagcagacatggct | |
| | gatatcccaggccacgtcttcacaagaggaatgatcatagctgttttgatcttggtggcagtagtgtgcctagtgactgtg | |
| | tgtgtcatttatagagttgacttggttctattttatagacatttaacgagaagagatgaaacattaacagatggaaaaacata | |
| | tgatgcttttgtgtcttacctaaaagaatgccgacctgaaaatggagaggagcacacctttgctgtggagattttgcccag | |
| | ggtgttggagaaacattttgggtataagttatgcatatttgaaagggatgtagtgcctggaggagctgttgttgatgaaatc | |
| | cactcactgatagagaaaagccgaagactaatcattgtcctaagtaaaagttatatgtctaatgaggtcaggtatgaactt | |
| | gaaagtggactccatgaagcattggtggaaagaaaaattaaaataatcttaattgaatttacacctgttactgacttcacatt | |
| | cttgccccaatcactaaagcttttgaaatctcacagagttctgaagtggaaggccgataaatctctttcttataactcaaggt | |
| | tctggaagaaccttctttacttaatgcctgcaaaaacagtcaagccaggtagagacgaaccggaagtcttgcctgttcttt | |
| | ccgagtcttgataa | |
| CAR E6 CD28- | atggccttaccagtgaccgccttgctcctgccgctggccttgctgctccacgccgccaggccggaggtgcagctggtg | 113 |
| CD28-CD28CD3 | gagtctgggggaggcctggtcaagcctggggggtccctgagactctcctgtgcagcctctggattcaccttcagtagg | |
| 3XFOXP3NFAT | tatggcatgagctgggtccgccaggctccagggaagaggctggagtgggtctcaaccattagtggcggaggcacct | |
| mCMV | acatatactacccagactcagtgaagggccgattcaccatctccagagacaacgccaagaacaccctgtatctgcaaat | |
| IL18Ralpha | gaacagcctgagagccgaggacacggctgtgtattactgtaccagagataactatggccgcaactatgattatggcatg | |
| | gattattggggccagggcaccctggtgaccgtgagcagcggtggtggcggttccggcggtggcggttccggtggtg | |
| | gcggttccgaaattgtgttgacacagtctccagccaccctgtctttgtctccaggggaaagagccaccctcacctgcag | |
| | cgccaccagcagtgttagctacatccactggtatcaacagaggcctggccagagccccaggctcctcatctatagcac | |
| | ctccaacctggccagcggcatcccagccaggttcagtggcagtgggtctgggagcgactacactctcaccatcagca | |
| | gcctagagcctgaagattttgcagtttattactgtcagcagcgtagcagctcccctttcacctttggcagcggcaccaaa | |
| | gtggaaattaaaaagcacctgtgtccttctccactgttccccggccctagcaagcctttctgggtcctggtggtggtggg | |
| | cggagtgctggcctgctacagcctgctggtgaccgtggcctttatcatcttctgggtgaggagtaagaggagcaggct | |
| | cctgcacagtgactacatgaacatgactcctagaagacctgggcctaccagaaagcattaccagccctatgccccacc | |
| | acgcgacttcgcagcctatcgctccagagtgaagttcagcaggagcgcagacgcccccgcgtacaagcagggcca | |
| | gaaccagctctataacgagctcaatctaggacgaagagaggagtacgatgttttggacaagagacgtggccgggacc | |
| | ctgagatggggggaaagccgagaaggaagaaccctcaggaaggcctgtacaatgaactgcagaaagataagatgg | |
| | cggaggcctacagtgagattgggatgaaaggcgagcgccggaggggcaaggggcacgatggcctttaccagggtc | |
| | tcagtacagccaccaaggacacctacgacgcccttcacatgcaggccctgccccctcgctgataagtttaaactgcca | |
| | gaacatttctctggcctaactggccggtaccggcttcattttttccatttactgcagaggcttcattttttccatttactgcaga | |
| | ggcttcattttttccatttactgcagaactagttaggcgtgtacggtgggaggcctatataagcagagctcgtttagtgaac | |
| | cgtcagatcgcctggagacgccatccacgctgttttgacctccatagaagacaccgggaccgatccagcctcgagag | |
| | acccaatgctagccaccatgaattgtagagaattacccttgaccctttgggtgcttatatctgtaagcactgcagaatctt | |
| | gtacttcacgtccccacattactgtggttgaaggggaacctttctatctgaaacattgctcgtgttcacttgcacatgagatt | |
| | gaaacaaccaccaaaagctggtacaaaagcagtggatcacaggaacatgtggagctgaacccaaggagttccagca | |
| | gaattgctttgcatgattgtgttttggagttttggccagttgagttgaatgacacaggatcttactttttccaaatgaaaaatta | |
| | tactcagaaatggaaattaaatgtcatcagaagaaataaacacagctgtttcactgaaagacaagtaactagtaaaattgt | |
| | ggaagttaaaaaattttttcagataacctgtgaaaacagttactatcaaacactggtcaacagcacatcattgtataagaac | |
| | tgtaaaaagctactactggagaacaataaaaacccaacgataaagaagaacgccgagtttgaagatcaggggtattac | |
| | tcctgcgtgcatttccttcatcataatggaaaactatttaatatcaccaaaaccttcaatataacaatagtggaagatcgcag | |
| | taatatagttccggttcttcttggaccaaagcttaaccatgttgcagtggaattaggaaaaaacgtaaggctcaactgctct | |
| | gctttgctgaatgaagaggatgtaatttattggatgttcggggaagaaaatggatcggatcctaatatacatgaagagaa | |
| | agaaatgagaattatgactccagaaggcaaatggcatgcttcaaaagtattgagaattgaaaatattggtgaaagcaatc | |
| | taaatgttttatataattgcactgtggccagcacgggaggcacagacaccaaaagcttcatcttggtgagaaaagcaga | |
| | catggctgatatcccaggccacgtcttcacaagaggaatgatcatagctgttttgatcttggtggcagtagtgtgcctagt | |
| | gactgtgtgtgtcatttatagagttgacttggttctattttatagacatttaacgagaagagatgaaacattaacagatggaa | |
| | aaacatatgatgcttttgtgtcttacctaaaagaatgccgacctgaaaatggagaggagcacacctttgctgtggagatttt | |
| | gcccagggtgttggagaaacattttgggtataagttatgcatatttgaaagggatgtagtgcctggaggagctgttgttga | |
| | tgaaatccactcactgatagagaaaagccgaagactaatcattgtcctaagtaaaagttatatgtctaatgaggtcaggta | |
| | tgaacttgaaagtggactccatgaagcattggtggaaagaaaaattaaaataatcttaattgaatttacacctgttactgac | |
| | ttcacattcttgccccaatcactaaagcttttgaaatctcacagagttctgaagtggaaggccgataaatctctttcttataac | |
| | tcaaggttctggaagaaccttctttacttaatgcctgcaaaaacagtcaagccaggtagagacgaaccggaagtcttgc | |
| | ctgttctttccgagtcttgataa | |
| CAR E6 CD28- | atggccttaccagtgaccgccttgctcctgccgctggccttgctgctccacgccgccaggccggaggtgcagctggtg | 114 |
| CD28-CD28CD3 | gagtctgggggaggcctggtcaagcctggggggtccctgagactctcctgtgcagcctctggattcaccttcagtagg | |
| 6XIL2NFAT | tatggcatgagctgggtccgccaggctccagggaagaggctggagtgggtctcaaccattagtggcggaggcacct | |
| mCMV | acatatactacccagactcagtgaagggccgattcaccatctccagagacaacgccaagaacaccctgtatctgcaaat | |
| IL18Ralpha | gaacagcctgagagccgaggacacggctgtgtattactgtaccagagataactatggccgcaactatgattatggcatg | |
| | gattattggggccagggcaccctggtgaccgtgagcagcggtggtggcggttccggcggtggcggttccggtggtg | |
| | gcggttccgaaattgtgttgacacagtctccagccaccctgtctttgtctccaggggaaagagccaccctcacctgcag | |
| | cgccaccagcagtgttagctacatccactggtatcaacagaggcctggccagagccccaggctcctcatctatagcac | |
| | ctccaacctggccagcggcatcccagccaggttcagtggcagtgggtctgggagcgactacactctcaccatcagca | |
| | gcctagagcctgaagattttgcagtttattactgtcagcagcgtagcagctcccctttcacctttggcagcggcaccaaa | |
| | gtggaaattaaaaagcacctgtgtccttctccactgttccccggccctagcaagcctttctgggtcctggtggtggtggg | |
| | cggagtgctggcctgctacagcctgctggtgaccgtggcctttatcatcttctgggtgaggagtaagaggagcaggct | |
| | cctgcacagtgactacatgaacatgactcctagaagacctgggcctaccagaaagcattaccagccctatgccccacc | |
| | acgcgacttcgcagcctatcgctccagagtgaagttcagcaggagcgcagacgcccccgcgtacaagcagggcca | |
| | gaaccagctctataacgagctcaatctaggacgaagagaggagtacgatgttttggacaagagacgtggccgggacc | |
| | ctgagatggggggaaagccgagaaggaagaaccctcaggaaggcctgtacaatgaactgcagaaagataagatgg | |
| | cggaggcctacagtgagattgggatgaaaggcgagcgccggaggggcaaggggcacgatggcctttaccagggtc | |
| | tcagtacagccaccaaggacacctacgacgcccttcacatgcaggccctgccccctcgctgataagtttaaactgcca | |
| | gaacatttctctggcctaactggccggtaccggaggaaaaactgtttcatacagaaggcgtggaggaaaaactgtttca | |
| | tacagaaggcgtggaggaaaaactgtttcatacagaaggcgtggaggaaaaactgtttcatacagaaggcgtggagg | |
| | aaaaactgtttcatacagaaggcgtggaggaaaaactgtttcatacagaaggcgtactagttaggcgtgtacggtggga | |
| | ggcctatataagcagagctcgtttagtgaaccgtcagatcgcctggagacgccatccacgctgttttgacctccatagaa | |
| | gacaccgggaccgatccagcctcgagagacccaatgctagccaccatgaattgtagagaattacccttgaccctttgg | |
| | gtgcttatatctgtaagcactgcagaatcttgtacttcacgtccccacattactgtggttgaaggggaacctttctatctgaa | |
| | acattgctcgtgttcacttgcacatgagattgaaacaaccaccaaaagctggtacaaaagcagtggatcacaggaacat | |
| | gtggagctgaacccaaggagttccagcagaattgctttgcatgattgtgttttggagttttggccagttgagttgaatgac | |
| | acaggatcttactttttccaaatgaaaaattatactcagaaatggaaattaaatgtcatcagaagaaataaacacagctgttt | |
| | cactgaaagacaagtaactagtaaaattgtggaagttaaaaaattttttcagataacctgtgaaaacagttactatcaaaca | |
| | ctggtcaacagcacatcattgtataagaactgtaaaaagctactactggagaacaataaaaacccaacgataaagaaga | |
| | acgccgagtttgaagatcaggggtattactcctgcgtgcatttccttcatcataatggaaaactatttaatatcaccaaaac | |
| | cttcaatataacaatagtggaagatcgcagtaatatagttccggttcttcttggaccaaagcttaaccatgttgcagtggaa | |
| | ttaggaaaaaacgtaaggctcaactgctctgctttgctgaatgaagaggatgtaatttattggatgttcggggaagaaaat | |
| | ggatcggatcctaatatacatgaagagaaagaaatgagaattatgactccagaaggcaaatggcatgcttcaaaagtat | |
| | tgagaattgaaaatattggtgaaagcaatctaaatgttttatataattgcactgtggccagcacgggaggcacagacacc | |
| | aaaagcttcatcttggtgagaaaagcagacatggctgatatcccaggccacgtcttcacaagaggaatgatcatagctg | |
| | ttttgatcttggtggcagtagtgtgcctagtgactgtgtgtgtcatttatagagttgacttggttctattttatagacatttaacg | |
| | agaagagatgaaacattaacagatggaaaaacatatgatgcttttgtgtcttacctaaaagaatgccgacctgaaaatgg | |
| | agaggagcacacctttgctgtggagattttgcccagggtgttggagaaacattttgggtataagttatgcatatttgaaag | |
| | ggatgtagtgcctggaggagctgttgttgatgaaatccactcactgatagagaaaagccgaagactaatcattgtcctaa | |
| | gtaaaagttatatgtctaatgaggtcaggtatgaacttgaaagtggactccatgaagcattggtggaaagaaaaattaaa | |
| | ataatcttaattgaatttacacctgttactgacttcacattcttgccccaatcactaaagcttttgaaatctcacagagttctga | |
| | agtggaaggccgataaatctctttcttataactcaaggttctggaagaaccttctttacttaatgcctgcaaaaacagtcaa | |
| | gccaggtagagacgaaccggaagtcttgcctgttctttccgagtcttgataa | |
| CAR E6 CD28- | atggccttaccagtgaccgccttgctcctgccgctggccttgctgctccacgccgccaggccggaggtgcagctggtg | 115 |
| CD28-CD28CD3 | gagtctgggggaggcctggtcaagcctggggggtccctgagactctcctgtgcagcctctggattcaccttcagtagg | |
| 3XIL2NFAT | tatggcatgagctgggtccgccaggctccagggaagaggctggagtgggtctcaaccattagtggcggaggcacct | |
| mCMV | acatatactacccagactcagtgaagggccgattcaccatctccagagacaacgccaagaacaccctgtatctgcaaat | |
| IL18Ralpha | gaacagcctgagagccgaggacacggctgtgtattactgtaccagagataactatggccgcaactatgattatggcatg | |
| | gattattggggccagggcaccctggtgaccgtgagcagcggtggtggcggttccggcggtggcggttccggtggtg | |
| | gcggttccgaaattgtgttgacacagtctccagccaccctgtctttgtctccaggggaaagagccaccctcacctgcag | |
| | cgccaccagcagtgttagctacatccactggtatcaacagaggcctggccagagccccaggctcctcatctatagcac | |
| | ctccaacctggccagcggcatcccagccaggttcagtggcagtgggtctgggagcgactacactctcaccatcagca | |
| | gcctagagcctgaagattttgcagtttattactgtcagcagcgtagcagctcccctttcacctttggcagcggcaccaaa | |
| | gtggaaattaaaaagcacctgtgtccttctccactgttccccggccctagcaagcctttctgggtcctggtggtggtggg | |
| | cggagtgctggcctgctacagcctgctggtgaccgtggcctttatcatcttctgggtgaggagtaagaggagcaggct | |
| | cctgcacagtgactacatgaacatgactcctagaagacctgggcctaccagaaagcattaccagccctatgccccacc | |
| | acgcgacttcgcagcctatcgctccagagtgaagttcagcaggagcgcagacgcccccgcgtacaagcagggcca | |
| | gaaccagctctataacgagctcaatctaggacgaagagaggagtacgatgttttggacaagagacgtggccgggacc | |
| | ctgagatggggggaaagccgagaaggaagaaccctcaggaaggcctgtacaatgaactgcagaaagataagatgg | |
| | cggaggcctacagtgagattgggatgaaaggcgagcgccggaggggcaaggggcacgatggcctttaccagggtc | |
| | tcagtacagccaccaaggacacctacgacgcccttcacatgcaggccctgccccctcgctgataagtttaaactgcca | |
| | gaacatttctctggcctaactggccggtaccggaggaaaaactgtttcatacagaaggcgtggaggaaaaactgtttca | |
| | tacagaaggcgtggaggaaaaactgtttcatacagaaggcgtactagttaggcgtgtacggtgggaggcctatataag | |
| | cagagctcgtttagtgaaccgtcagatcgcctggagacgccatccacgctgttttgacctccatagaagacaccggga | |
| | ccgatccagcctcgagagacccaatgctagccaccatgaattgtagagaattacccttgaccctttgggtgcttatatct | |
| | gtaagcactgcagaatcttgtacttcacgtccccacattactgtggttgaaggggaacctttctatctgaaacattgctcgt | |
| | gttcacttgcacatgagattgaaacaaccaccaaaagctggtacaaaagcagtggatcacaggaacatgtggagctga | |
| | acccaaggagttccagcagaattgctttgcatgattgtgttttggagttttggccagttgagttgaatgacacaggatctta | |
| | ctttttccaaatgaaaaattatactcagaaatggaaattaaatgtcatcagaagaaataaacacagctgtttcactgaaaga | |
| | caagtaactagtaaaattgtggaagttaaaaaattttttcagataacctgtgaaaacagttactatcaaacactggtcaaca | |
| | gcacatcattgtataagaactgtaaaaagctactactggagaacaataaaaacccaacgataaagaagaacgccgagt | |
| | ttgaagatcaggggtattactcctgcgtgcatttccttcatcataatggaaaactatttaatatcaccaaaaccttcaatataa | |
| | caatagtggaagatcgcagtaatatagttccggttcttcttggaccaaagcttaaccatgttgcagtggaattaggaaaaa | |
| | acgtaaggctcaactgctctgctttgctgaatgaagaggatgtaatttattggatgttcggggaagaaaatggatcggat | |
| | cctaatatacatgaagagaaagaaatgagaattatgactccagaaggcaaatggcatgcttcaaaagtattgagaattga | |
| | aaatattggtgaaagcaatctaaatgttttatataattgcactgtggccagcacgggaggcacagacaccaaaagcttca | |
| | tcttggtgagaaaagcagacatggctgatatcccaggccacgtcttcacaagaggaatgatcatagctgttttgatcttgg | |
| | tggcagtagtgtgcctagtgactgtgtgtgtcatttatagagttgacttggttctattttatagacatttaacgagaagagatg | |
| | aaacattaacagatggaaaaacatatgatgcttttgtgtcttacctaaaagaatgccgacctgaaaatggagaggagcac | |
| | acctttgctgtggagattttgcccagggtgttggagaaacattttgggtataagttatgcatatttgaaagggatgtagtgc | |
| | ctggaggagctgttgttgatgaaatccactcactgatagagaaaagccgaagactaatcattgtcctaagtaaaagttata | |
| | tgtctaatgaggtcaggtatgaacttgaaagtggactccatgaagcattggtggaaagaaaaattaaaataatcttaattg | |
| | aatttacacctgttactgacttcacattcttgccccaatcactaaagcttttgaaatctcacagagttctgaagtggaaggcc | |
| | gataaatctctttcttataactcaaggttctggaagaaccttctttacttaatgcctgcaaaaacagtcaagccaggtagag | |
| | acgaaccggaagtcttgcctgttctttccgagtcttgataa | |
| CAR E6 CD28- | atggccttaccagtgaccgccttgctcctgccgctggccttgctgctccacgccgccaggccggaggtgcagctggtg | 116 |
| CD28-CD281XX | gagtctgggggaggcctggtcaagcctggggggtccctgagactctcctgtgcagcctctggattcaccttcagtagg | |
| 6XFOXP3NFAT | tatggcatgagctgggtccgccaggctccagggaagaggctggagtgggtctcaaccattagtggcggaggcacct | |
| mCMV | acatatactacccagactcagtgaagggccgattcaccatctccagagacaacgccaagaacaccctgtatctgcaaat | |
| IL18Ralpha | gaacagcctgagagccgaggacacggctgtgtattactgtaccagagataactatggccgcaactatgattatggcatg | |
| | gattattggggccagggcaccctggtgaccgtgagcagcggtggtggcggttccggcggtggcggttccggtggtg | |
| | gcggttccgaaattgtgttgacacagtctccagccaccctgtctttgtctccaggggaaagagccaccctcacctgcag | |
| | cgccaccagcagtgttagctacatccactggtatcaacagaggcctggccagagccccaggctcctcatctatagcac | |
| | ctccaacctggccagcggcatcccagccaggttcagtggcagtgggtctgggagcgactacactctcaccatcagca | |
| | gcctagagcctgaagattttgcagtttattactgtcagcagcgtagcagctcccctttcacctttggcagcggcaccaaa | |
| | gtggaaattaaaaagcacctgtgtccttctccactgttccccggccctagcaagcctttctgggtcctggtggtggtggg | |
| | cggagtgctggcctgctacagcctgctggtgaccgtggcctttatcatcttctgggtgaggagtaagaggagcaggct | |
| | cctgcacagtgactacatgaacatgactcctagaagacctgggcctaccagaaagcattaccagccctatgccccacc | |
| | acgcgacttcgcagcctatcgctccagagtgaagttcagcaggagcgcagacgcccccgcgtacaagcagggcca | |
| | gaaccagctctataacgagctcaatctaggacgaagagaggagtacgatgttttggacaagagacgtggccgggacc | |
| | ctgagatggggggaaagccgagaaggaagaaccctcaggaaggcctgttcaatgaactgcagaaagataagatgg | |
| | cggaggccttcagtgagattgggatgaaaggcgagcgccggaggggcaaggggcacgatggccttttccagggtct | |
| | cagtacagccaccaaggacaccttcgacgcccttcacatgcaggccctgccccctcgctgataagtttaaactgccag | |
| | aacattactctggcctaactggccggtaccggcttcattttttccatttactgcagaggcttcattttttccatttactgcagag | |
| | gcttcattttttccatttactgcagaggcttcattttttccatttactgcagaggcttcattttttccatttactgcagaggcttcat | |
| | tttttccatttactgcagaactagttaggcgtgtacggtgggaggcctatataagcagagctcgtttagtgaaccgtcaga | |
| | tcgcctggagacgccatccacgctgttttgacctccatagaagacaccgggaccgatccagcctcgagagacccaat | |
| | gctagccaccatgaattgtagagaattacccttgaccctttgggtgcttatatctgtaagcactgcagaatcttgtacttca | |
| | cgtccccacattactgtggttgaaggggaacctttctatctgaaacattgctcgtgttcacttgcacatgagattgaaacaa | |
| | ccaccaaaagctggtacaaaagcagtggatcacaggaacatgtggagctgaacccaaggagttccagcagaattgct | |
| | ttgcatgattgtgttttggagttttggccagttgagttgaatgacacaggatcttactttttccaaatgaaaaattatactcaga | |
| | aatggaaattaaatgtcatcagaagaaataaacacagctgtttcactgaaagacaagtaactagtaaaattgtggaagtta | |
| | aaaaattttttcagataacctgtgaaaacagttactatcaaacactggtcaacagcacatcattgtataagaactgtaaaaa | |
| | gctactactggagaacaataaaaacccaacgataaagaagaacgccgagtttgaagatcaggggtattactcctgcgt | |
| | gcatttccttcatcataatggaaaactatttaatatcaccaaaaccttcaatataacaatagtggaagatcgcagtaatatag | |
| | ttccggttcttcttggaccaaagcttaaccatgttgcagtggaattaggaaaaaacgtaaggctcaactgctctgctttgct | |
| | gaatgaagaggatgtaatttattggatgttcggggaagaaaatggatcggatcctaatatacatgaagagaaagaaatg | |
| | agaattatgactccagaaggcaaatggcatgcttcaaaagtattgagaattgaaaatattggtgaaagcaatctaaatgtt | |
| | ttatataattgcactgtggccagcacgggaggcacagacaccaaaagcttcatcttggtgagaaaagcagacatggct | |
| | gatatcccaggccacgtcttcacaagaggaatgatcatagctgttttgatcttggtggcagtagtgtgcctagtgactgtg | |
| | tgtgtcatttatagagttgacttggttctattttatagacatttaacgagaagagatgaaacattaacagatggaaaaacata | |
| | tgatgcttttgtgtcttacctaaaagaatgccgacctgaaaatggagaggagcacacctttgctgtggagattttgcccag | |
| | ggtgttggagaaacattttgggtataagttatgcatatttgaaagggatgtagtgcctggaggagctgttgttgatgaaatc | |
| | cactcactgatagagaaaagccgaagactaatcattgtcctaagtaaaagttatatgtctaatgaggtcaggtatgaactt | |
| | gaaagtggactccatgaagcattggtggaaagaaaaattaaaataatcttaattgaatttacacctgttactgacttcacatt | |
| | cttgccccaatcactaaagcttttgaaatctcacagagttctgaagtggaaggccgataaatctctttcttataactcaaggt | |
| | tctggaagaaccttctttacttaatgcctgcaaaaacagtcaagccaggtagagacgaaccggaagtcttgcctgttcttt | |
| | ccgagtcttgataa | |
| CAR E6 CD28- | atggccttaccagtgaccgccttgctcctgccgctggccttgctgctccacgccgccaggccggaggtgcagctggtg | 117 |
| CD28-CD281XX | gagtctgggggaggcctggtcaagcctggggggtccctgagactctcctgtgcagcctctggattcaccttcagtagg | |
| 3XFOXP3NFAT | tatggcatgagctgggtccgccaggctccagggaagaggctggagtgggtctcaaccattagtggcggaggcacct | |
| mCMV | acatatactacccagactcagtgaagggccgattcaccatctccagagacaacgccaagaacaccctgtatctgcaaat | |
| IL18Ralpha | gaacagcctgagagccgaggacacggctgtgtattactgtaccagagataactatggccgcaactatgattatggcatg | |
| | gattattggggccagggcaccctggtgaccgtgagcagcggtggtggcggttccggcggtggcggttccggtggtg | |
| | gcggttccgaaattgtgttgacacagtctccagccaccctgtctttgtctccaggggaaagagccaccctcacctgcag | |
| | cgccaccagcagtgttagctacatccactggtatcaacagaggcctggccagagccccaggctcctcatctatagcac | |
| | ctccaacctggccagcggcatcccagccaggttcagtggcagtgggtctgggagcgactacactctcaccatcagca | |
| | gcctagagcctgaagattttgcagtttattactgtcagcagcgtagcagctcccctttcacctttggcagcggcaccaaa | |
| | gtggaaattaaaaagcacctgtgtccttctccactgttccccggccctagcaagcctttctgggtcctggtggtggtggg | |
| | cggagtgctggcctgctacagcctgctggtgaccgtggcctttatcatcttctgggtgaggagtaagaggagcaggct | |
| | cctgcacagtgactacatgaacatgactcctagaagacctgggcctaccagaaagcattaccagccctatgccccacc | |
| | acgcgacttcgcagcctatcgctccagagtgaagttcagcaggagcgcagacgcccccgcgtacaagcagggcca | |
| | gaaccagctctataacgagctcaatctaggacgaagagaggagtacgatgttttggacaagagacgtggccgggacc | |
| | ctgagatggggggaaagccgagaaggaagaaccctcaggaaggcctgttcaatgaactgcagaaagataagatgg | |
| | cggaggccttcagtgagattgggatgaaaggcgagcgccggaggggcaaggggcacgatggccttttccagggtct | |
| | cagtacagccaccaaggacaccttcgacgcccttcacatgcaggccctgccccctcgctgataagtttaaactgccag | |
| | aacattactctggcctaactggccggtaccggcttcattttttccatttactgcagaggcttcattttttccatttactgcagag | |
| | gcttcattttttccatttactgcagaactagttaggcgtgtacggtgggaggcctatataagcagagctcgtttagtgaacc | |
| | gtcagatcgcctggagacgccatccacgctgttttgacctccatagaagacaccgggaccgatccagcctcgagaga | |
| | cccaatgctagccaccatgaattgtagagaattacccttgaccctttgggtgcttatatctgtaagcactgcagaatcttgt | |
| | acttcacgtccccacattactgtggttgaaggggaacctttctatctgaaacattgctcgtgttcacttgcacatgagattga | |
| | aacaaccaccaaaagctggtacaaaagcagtggatcacaggaacatgtggagctgaacccaaggagttccagcaga | |
| | attgctttgcatgattgtgttttggagttttggccagttgagttgaatgacacaggatcttactttttccaaatgaaaaattata | |
| | ctcagaaatggaaattaaatgtcatcagaagaaataaacacagctgtttcactgaaagacaagtaactagtaaaattgtg | |
| | gaagttaaaaaattttttcagataacctgtgaaaacagttactatcaaacactggtcaacagcacatcattgtataagaact | |
| | gtaaaaagctactactggagaacaataaaaacccaacgataaagaagaacgccgagtttgaagatcaggggtattact | |
| | cctgcgtgcatttccttcatcataatggaaaactatttaatatcaccaaaaccttcaatataacaatagtggaagatcgcagt | |
| | aatatagttccggttcttcttggaccaaagcttaaccatgttgcagtggaattaggaaaaaacgtaaggctcaactgctct | |
| | gctttgctgaatgaagaggatgtaatttattggatgttcggggaagaaaatggatcggatcctaatatacatgaagagaa | |
| | agaaatgagaattatgactccagaaggcaaatggcatgcttcaaaagtattgagaattgaaaatattggtgaaagcaatc | |
| | taaatgttttatataattgcactgtggccagcacgggaggcacagacaccaaaagcttcatcttggtgagaaaagcaga | |
| | catggctgatatcccaggccacgtcttcacaagaggaatgatcatagctgttttgatcttggtggcagtagtgtgcctagt | |
| | gactgtgtgtgtcatttatagagttgacttggttctattttatagacatttaacgagaagagatgaaacattaacagatggaa | |
| | aaacatatgatgcttttgtgtcttacctaaaagaatgccgacctgaaaatggagaggagcacacctttgctgtggagatttt | |
| | gcccagggtgttggagaaacattttgggtataagttatgcatatttgaaagggatgtagtgcctggaggagctgttgttga | |
| | tgaaatccactcactgatagagaaaagccgaagactaatcattgtcctaagtaaaagttatatgtctaatgaggtcaggta | |
| | tgaacttgaaagtggactccatgaagcattggtggaaagaaaaattaaaataatcttaattgaatttacacctgttactgac | |
| | ttcacattcttgccccaatcactaaagcttttgaaatctcacagagttctgaagtggaaggccgataaatctctttcttataac | |
| | tcaaggttctggaagaaccttctttacttaatgcctgcaaaaacagtcaagccaggtagagacgaaccggaagtcttgc | |
| | ctgttctttccgagtcttgataa | |
| CAR E6 CD28- | atggccttaccagtgaccgccttgctcctgccgctggccttgctgctccacgccgccaggccggaggtgcagctggtg | 118 |
| CD28-CD281XX | gagtctgggggaggcctggtcaagcctggggggtccctgagactctcctgtgcagcctctggattcaccttcagtagg | |
| 6XIL2NFAT | tatggcatgagctgggtccgccaggctccagggaagaggctggagtgggtctcaaccattagtggcggaggcacct | |
| mCMV | acatatactacccagactcagtgaagggccgattcaccatctccagagacaacgccaagaacaccctgtatctgcaaat | |
| IL18Ralpha | gaacagcctgagagccgaggacacggctgtgtattactgtaccagagataactatggccgcaactatgattatggcatg | |
| | gattattggggccagggcaccctggtgaccgtgagcagcggtggtggcggttccggcggtggcggttccggtggtg | |
| | gcggttccgaaattgtgttgacacagtctccagccaccctgtctttgtctccaggggaaagagccaccctcacctgcag | |
| | cgccaccagcagtgttagctacatccactggtatcaacagaggcctggccagagccccaggctcctcatctatagcac | |
| | ctccaacctggccagcggcatcccagccaggttcagtggcagtgggtctgggagcgactacactctcaccatcagca | |
| | gcctagagcctgaagattttgcagtttattactgtcagcagcgtagcagctcccctttcacctttggcagcggcaccaaa | |
| | gtggaaattaaaaagcacctgtgtccttctccactgttccccggccctagcaagcctttctgggtcctggtggtggtggg | |
| | cggagtgctggcctgctacagcctgctggtgaccgtggcctttatcatcttctgggtgaggagtaagaggagcaggct | |
| | cctgcacagtgactacatgaacatgactcctagaagacctgggcctaccagaaagcattaccagccctatgccccacc | |
| | acgcgacttcgcagcctatcgctccagagtgaagttcagcaggagcgcagacgcccccgcgtacaagcagggcca | |
| | gaaccagctctataacgagctcaatctaggacgaagagaggagtacgatgttttggacaagagacgtggccgggacc | |
| | ctgagatggggggaaagccgagaaggaagaaccctcaggaaggcctgttcaatgaactgcagaaagataagatgg | |
| | cggaggccttcagtgagattgggatgaaaggcgagcgccggaggggcaaggggcacgatggccttttccagggtct | |
| | cagtacagccaccaaggacaccttcgacgcccttcacatgcaggccctgccccctcgctgataagtttaaactgccag | |
| | aacattactctggcctaactggccggtaccggaggaaaaactgtttcatacagaaggcgtggaggaaaaactgtttcat | |
| | acagaaggcgtggaggaaaaactgtttcatacagaaggcgtggaggaaaaactgtttcatacagaaggcgtggagga | |
| | aaaactgtttcatacagaaggcgtggaggaaaaactgtttcatacagaaggcgtactagttaggcgtgtacggtgggag | |
| | gcctatataagcagagctcgtttagtgaaccgtcagatcgcctggagacgccatccacgctgttttgacctccatagaag | |
| | acaccgggaccgatccagcctcgagagacccaatgctagccaccatgaattgtagagaattacccttgaccctttggg | |
| | tgcttatatctgtaagcactgcagaatcttgtacttcacgtccccacattactgtggttgaaggggaacctttctatctgaaa | |
| | cattgctcgtgttcacttgcacatgagattgaaacaaccaccaaaagctggtacaaaagcagtggatcacaggaacatg | |
| | tggagctgaacccaaggagttccagcagaattgctttgcatgattgtgttttggagttttggccagttgagttgaatgacac | |
| | aggatcttactttttccaaatgaaaaattatactcagaaatggaaattaaatgtcatcagaagaaataaacacagctgtttca | |
| | ctgaaagacaagtaactagtaaaattgtggaagttaaaaaattttttcagataacctgtgaaaacagttactatcaaacact | |
| | ggtcaacagcacatcattgtataagaactgtaaaaagctactactggagaacaataaaaacccaacgataaagaagaa | |
| | cgccgagtttgaagatcaggggtattactcctgcgtgcatttccttcatcataatggaaaactatttaatatcaccaaaacct | |
| | tcaatataacaatagtggaagatcgcagtaatatagttccggttcttcttggaccaaagcttaaccatgttgcagtggaatt | |
| | aggaaaaaacgtaaggctcaactgctctgctttgctgaatgaagaggatgtaatttattggatgttcggggaagaaaatg | |
| | gatcggatcctaatatacatgaagagaaagaaatgagaattatgactccagaaggcaaatggcatgcttcaaaagtatt | |
| | gagaattgaaaatattggtgaaagcaatctaaatgttttatataattgcactgtggccagcacgggaggcacagacacca | |
| | aaagcttcatcttggtgagaaaagcagacatggctgatatcccaggccacgtcttcacaagaggaatgatcatagctgtt | |
| | ttgatcttggtggcagtagtgtgcctagtgactgtgtgtgtcatttatagagttgacttggttctattttatagacatttaacga | |
| | gaagagatgaaacattaacagatggaaaaacatatgatgcttttgtgtcttacctaaaagaatgccgacctgaaaatgga | |
| | gaggagcacacctttgctgtggagattttgcccagggtgttggagaaacattttgggtataagttatgcatatttgaaagg | |
| | gatgtagtgcctggaggagctgttgttgatgaaatccactcactgatagagaaaagccgaagactaatcattgtcctaag | |
| | taaaagttatatgtctaatgaggtcaggtatgaacttgaaagtggactccatgaagcattggtggaaagaaaaattaaaat | |
| | aatcttaattgaatttacacctgttactgacttcacattcttgccccaatcactaaagcttttgaaatctcacagagttctgaag | |
| | tggaaggccgataaatctctttcttataactcaaggttctggaagaaccttctttacttaatgcctgcaaaaacagtcaagc | |
| | caggtagagacgaaccggaagtcttgcctgttctttccgagtcttgataa | |
| CAR E6 CD28- | atggccttaccagtgaccgccttgctcctgccgctggccttgctgctccacgccgccaggccggaggtgcagctggtg | 119 |
| CD28-CD281XX | gagtctgggggaggcctggtcaagcctggggggtccctgagactctcctgtgcagcctctggattcaccttcagtagg | |
| 3XIL2NFAT | tatggcatgagctgggtccgccaggctccagggaagaggctggagtgggtctcaaccattagtggcggaggcacct | |
| mCMV | acatatactacccagactcagtgaagggccgattcaccatctccagagacaacgccaagaacaccctgtatctgcaaat | |
| IL18Ralpha | gaacagcctgagagccgaggacacggctgtgtattactgtaccagagataactatggccgcaactatgattatggcatg | |
| | gattattggggccagggcaccctggtgaccgtgagcagcggtggtggcggttccggcggtggcggttccggtggtg | |
| | gcggttccgaaattgtgttgacacagtctccagccaccctgtctttgtctccaggggaaagagccaccctcacctgcag | |
| | cgccaccagcagtgttagctacatccactggtatcaacagaggcctggccagagccccaggctcctcatctatagcac | |
| | ctccaacctggccagcggcatcccagccaggttcagtggcagtgggtctgggagcgactacactctcaccatcagca | |
| | gcctagagcctgaagattttgcagtttattactgtcagcagcgtagcagctcccctttcacctttggcagcggcaccaaa | |
| | gtggaaattaaaaagcacctgtgtccttctccactgttccccggccctagcaagcctttctgggtcctggtggtggtggg | |
| | cggagtgctggcctgctacagcctgctggtgaccgtggcctttatcatcttctgggtgaggagtaagaggagcaggct | |
| | cctgcacagtgactacatgaacatgactcctagaagacctgggcctaccagaaagcattaccagccctatgccccacc | |
| | acgcgacttcgcagcctatcgctccagagtgaagttcagcaggagcgcagacgcccccgcgtacaagcagggcca | |
| | gaaccagctctataacgagctcaatctaggacgaagagaggagtacgatgttttggacaagagacgtggccgggacc | |
| | ctgagatggggggaaagccgagaaggaagaaccctcaggaaggcctgttcaatgaactgcagaaagataagatgg | |
| | cggaggccttcagtgagattgggatgaaaggcgagcgccggaggggcaaggggcacgatggccttttccagggtct | |
| | cagtacagccaccaaggacaccttcgacgcccttcacatgcaggccctgccccctcgctgataagtttaaactgccag | |
| | aacattactctggcctaactggccggtaccggaggaaaaactgtttcatacagaaggcgtggaggaaaaactgtttcat | |
| | acagaaggcgtggaggaaaaactgtttcatacagaaggcgtactagttaggcgtgtacggtgggaggcctatataagc | |
| | agagctcgtttagtgaaccgtcagatcgcctggagacgccatccacgctgttttgacctccatagaagacaccgggac | |
| | cgatccagcctcgagagacccaatgctagccaccatgaattgtagagaattacccttgaccctttgggtgcttatatctg | |
| | taagcactgcagaatcttgtacttcacgtccccacattactgtggttgaaggggaacctttctatctgaaacattgctcgtgt | |
| | tcacttgcacatgagattgaaacaaccaccaaaagctggtacaaaagcagtggatcacaggaacatgtggagctgaac | |
| | ccaaggagttccagcagaattgctttgcatgattgtgttttggagttttggccagttgagttgaatgacacaggatcttactt | |
| | tttccaaatgaaaaattatactcagaaatggaaattaaatgtcatcagaagaaataaacacagctgtttcactgaaagaca | |
| | agtaactagtaaaattgtggaagttaaaaaattttttcagataacctgtgaaaacagttactatcaaacactggtcaacagc | |
| | acatcattgtataagaactgtaaaaagctactactggagaacaataaaaacccaacgataaagaagaacgccgagtttg | |
| | aagatcaggggtattactcctgcgtgcatttccttcatcataatggaaaactatttaatatcaccaaaaccttcaatataaca | |
| | atagtggaagatcgcagtaatatagttccggttcttcttggaccaaagcttaaccatgttgcagtggaattaggaaaaaac | |
| | gtaaggctcaactgctctgctttgctgaatgaagaggatgtaatttattggatgttcggggaagaaaatggatcggatcct | |
| | aatatacatgaagagaaagaaatgagaattatgactccagaaggcaaatggcatgcttcaaaagtattgagaattgaaa | |
| | atattggtgaaagcaatctaaatgttttatataattgcactgtggccagcacgggaggcacagacaccaaaagcttcatct | |
| | tggtgagaaaagcagacatggctgatatcccaggccacgtcttcacaagaggaatgatcatagctgttttgatcttggtg | |
| | gcagtagtgtgcctagtgactgtgtgtgtcatttatagagttgacttggttctattttatagacatttaacgagaagagatga | |
| | aacattaacagatggaaaaacatatgatgcttttgtgtcttacctaaaagaatgccgacctgaaaatggagaggagcaca | |
| | cctttgctgtggagattttgcccagggtgttggagaaacattttgggtataagttatgcatatttgaaagggatgtagtgcct | |
| | ggaggagctgttgttgatgaaatccactcactgatagagaaaagccgaagactaatcattgtcctaagtaaaagttatat | |
| | gtctaatgaggtcaggtatgaacttgaaagtggactccatgaagcattggtggaaagaaaaattaaaataatcttaattga | |
| | atttacacctgttactgacttcacattcttgccccaatcactaaagcttttgaaatctcacagagttctgaagtggaaggccg | |
| | ataaatctctttcttataactcaaggttctggaagaaccttctttacttaatgcctgcaaaaacagtcaagccaggtagaga | |
| | cgaaccggaagtcttgcctgttctttccgagtcttgataa | |
| CAR 20A10N | atggccttaccagtgaccgccttgctcctgccgctggccttgctgctccacgccgccaggccgcaggtgcagctggtt | 120 |
| CD8-CD8-41BB | gaatctggcggcggacttgtgaagcctggcggatctctgagactgagctgtgccgccagcggcttcacctttagcacat | |
| CD3 | acgccatgagctggatcagacaggcccctggcaaaggcctggaatgggtggcgtctattggcagagccggcagcac | |
| 6XFOXP3NFAT | ctactacagcgactctgtgaagggcagattcaccatcagccgggacaacgccaagaacagcctgtacctgcagatga | |
| mCMV | actccctgagagccgaggacaccgccgtgtactattgtgccagaggacccatctacaacgactacgacgagttcgcct | |
| IL18Ralpha | attggggccagggcacactggtcacagtttctagcggcggtggcggaagcggaggcggtggctccggtggcggag | |
| | gcagcgaaattgtgctgacacagagccccgccacactgtcactttctccaggcgaaagagccacactgagctgcaag | |
| | agcagccagagcgtgctgtactccagcaaccagaagaactacctggcctggtatcagcagaagcccggccaagctc | |
| | ctcggctgctgatctattgggccagcacaagagagagcggcatccctgccagattttctggcagcggctctggcaccg | |
| | atttcaccctgaccataagcagcctggaacctgaggacttcgccgtgtattactgccaccagtacctgagcagcctgac | |
| | ctttggcggaggcaccaaggtggaaatcaagcggacaacaacccctgcccccagacctcctaccccagcccctaca | |
| | attgccagccagcctctgagcctgaggcccgaggcttgtagacctgctgctggcggagccgtgcacaccagaggact | |
| | ggatttcgcctgcgacatctacatctgggcgcccttggccgggacttgtggggtccttctcctgtcactggttatcaccct | |
| | ttactgcaaacggggcagaaagaaactcctgtatatattcaaacaaccatttatgagaccagtacaaactactcaagagg | |
| | aagatggctgtagctgccgatttccagaagaagaagaaggaggatgtgaactgagagtgaagttcagcaggagcgc | |
| | agacgcccccgcgtacaagcagggccagaaccagctctataacgagctcaatctaggacgaagagaggagtacgat | |
| | gttttggacaagagacgtggccgggaccctgagatggggggaaagccgagaaggaagaaccctcaggaaggcctg | |
| | tacaatgaactgcagaaagataagatggcggaggcctacagtgagattgggatgaaaggcgagcgccggaggggc | |
| | aaggggcacgatggcctttaccagggtctcagtacagccaccaaggacacctacgacgcccttcacatgcaggccct | |
| | gccccctcgctgataagtttaaactgccagaacatttctctggcctaactggccggtaccggcttcattttttccatttactg | |
| | cagaggcttcattttttccatttactgcagaggcttcattttttccatttactgcagaggcttcattttttccatttactgcagag | |
| | gcttcattttttccatttactgcagaggcttcattttttccatttactgcagaactagttaggcgtgtacggtgggaggcctat | |
| | ataagcagagctcgtttagtgaaccgtcagatcgcctggagacgccatccacgctgttttgacctccatagaagacacc | |
| | gggaccgatccagcctcgagagacccaatgctagccaccatgaattgtagagaattacccttgaccctttgggtgctta | |
| | tatctgtaagcactgcagaatcttgtacttcacgtccccacattactgtggttgaaggggaacctttctatctgaaacattgc | |
| | tcgtgttcacttgcacatgagattgaaacaaccaccaaaagctggtacaaaagcagtggatcacaggaacatgtggag | |
| | ctgaacccaaggagttccagcagaattgctttgcatgattgtgttttggagttttggccagttgagttgaatgacacaggat | |
| | cttactttttccaaatgaaaaattatactcagaaatggaaattaaatgtcatcagaagaaataaacacagctgtttcactgaa | |
| | agacaagtaactagtaaaattgtggaagttaaaaaattttttcagataacctgtgaaaacagttactatcaaacactggtca | |
| | acagcacatcattgtataagaactgtaaaaagctactactggagaacaataaaaacccaacgataaagaagaacgccg | |
| | agtttgaagatcaggggtattactcctgcgtgcatttccttcatcataatggaaaactatttaatatcaccaaaaccttcaata | |
| | taacaatagtggaagatcgcagtaatatagttccggttcttcttggaccaaagcttaaccatgttgcagtggaattaggaa | |
| | aaaacgtaaggctcaactgctctgctttgctgaatgaagaggatgtaatttattggatgttcggggaagaaaatggatcg | |
| | gatcctaatatacatgaagagaaagaaatgagaattatgactccagaaggcaaatggcatgcttcaaaagtattgagaat | |
| | tgaaaatattggtgaaagcaatctaaatgttttatataattgcactgtggccagcacgggaggcacagacaccaaaagct | |
| | tcatcttggtgagaaaagcagacatggctgatatcccaggccacgtcttcacaagaggaatgatcatagctgttttgatct | |
| | tggtggcagtagtgtgcctagtgactgtgtgtgtcatttatagagttgacttggttctattttatagacatttaacgagaaga | |
| | gatgaaacattaacagatggaaaaacatatgatgcttttgtgtcttacctaaaagaatgccgacctgaaaatggagagga | |
| | gcacacctttgctgtggagattttgcccagggtgttggagaaacattttgggtataagttatgcatatttgaaagggatgta | |
| | gtgcctggaggagctgttgttgatgaaatccactcactgatagagaaaagccgaagactaatcattgtcctaagtaaaag | |
| | ttatatgtctaatgaggtcaggtatgaacttgaaagtggactccatgaagcattggtggaaagaaaaattaaaataatctta | |
| | attgaatttacacctgttactgacttcacattcttgccccaatcactaaagcttttgaaatctcacagagttctgaagtggaa | |
| | ggccgataaatctctttcttataactcaaggttctggaagaaccttctttacttaatgcctgcaaaaacagtcaagccaggt | |
| | agagacgaaccggaagtcttgcctgttctttccgagtcttgataa | |
| CAR 20A10N | atggccttaccagtgaccgccttgctcctgccgctggccttgctgctccacgccgccaggccgcaggtgcagctggtt | 121 |
| CD8-CD8-41BB | gaatctggcggcggacttgtgaagcctggcggatctctgagactgagctgtgccgccagcggcttcacctttagcacat | |
| CD3 | acgccatgagctggatcagacaggcccctggcaaaggcctggaatgggtggcgtctattggcagagccggcagcac | |
| 3XFOXP3NFAT | ctactacagcgactctgtgaagggcagattcaccatcagccgggacaacgccaagaacagcctgtacctgcagatga | |
| mCMV | actccctgagagccgaggacaccgccgtgtactattgtgccagaggacccatctacaacgactacgacgagttcgcct | |
| IL18Ralpha | attggggccagggcacactggtcacagtttctagcggcggtggcggaagcggaggcggtggctccggtggcggag | |
| | gcagcgaaattgtgctgacacagagccccgccacactgtcactttctccaggcgaaagagccacactgagctgcaag | |
| | agcagccagagcgtgctgtactccagcaaccagaagaactacctggcctggtatcagcagaagcccggccaagctc | |
| | ctcggctgctgatctattgggccagcacaagagagagcggcatccctgccagattttctggcagcggctctggcaccg | |
| | atttcaccctgaccataagcagcctggaacctgaggacttcgccgtgtattactgccaccagtacctgagcagcctgac | |
| | ctttggcggaggcaccaaggtggaaatcaagcggacaacaacccctgcccccagacctcctaccccagcccctaca | |
| | attgccagccagcctctgagcctgaggcccgaggcttgtagacctgctgctggcggagccgtgcacaccagaggact | |
| | ggatttcgcctgcgacatctacatctgggcgcccttggccgggacttgtggggtccttctcctgtcactggttatcaccct | |
| | ttactgcaaacggggcagaaagaaactcctgtatatattcaaacaaccatttatgagaccagtacaaactactcaagagg | |
| | aagatggctgtagctgccgatttccagaagaagaagaaggaggatgtgaactgagagtgaagttcagcaggagcgc | |
| | agacgcccccgcgtacaagcagggccagaaccagctctataacgagctcaatctaggacgaagagaggagtacgat | |
| | gttttggacaagagacgtggccgggaccctgagatggggggaaagccgagaaggaagaaccctcaggaaggcctg | |
| | tacaatgaactgcagaaagataagatggcggaggcctacagtgagattgggatgaaaggcgagcgccggaggggc | |
| | aaggggcacgatggcctttaccagggtctcagtacagccaccaaggacacctacgacgcccttcacatgcaggccct | |
| | gccccctcgctgataagtttaaactgccagaacatttctctggcctaactggccggtaccggcttcattttttccatttactg | |
| | cagaggcttcattttttccatttactgcagaggcttcattttttccatttactgcagaactagttaggcgtgtacggtgggag | |
| | gcctatataagcagagctcgtttagtgaaccgtcagatcgcctggagacgccatccacgctgttttgacctccatagaag | |
| | acaccgggaccgatccagcctcgagagacccaatgctagccaccatgaattgtagagaattacccttgaccctttggg | |
| | tgcttatatctgtaagcactgcagaatcttgtacttcacgtccccacattactgtggttgaaggggaacctttctatctgaaa | |
| | cattgctcgtgttcacttgcacatgagattgaaacaaccaccaaaagctggtacaaaagcagtggatcacaggaacatg | |
| | tggagctgaacccaaggagttccagcagaattgctttgcatgattgtgttttggagttttggccagttgagttgaatgacac | |
| | aggatcttactttttccaaatgaaaaattatactcagaaatggaaattaaatgtcatcagaagaaataaacacagctgtttca | |
| | ctgaaagacaagtaactagtaaaattgtggaagttaaaaaattttttcagataacctgtgaaaacagttactatcaaacact | |
| | ggtcaacagcacatcattgtataagaactgtaaaaagctactactggagaacaataaaaacccaacgataaagaagaa | |
| | cgccgagtttgaagatcaggggtattactcctgcgtgcatttccttcatcataatggaaaactatttaatatcaccaaaacct | |
| | tcaatataacaatagtggaagatcgcagtaatatagttccggttcttcttggaccaaagcttaaccatgttgcagtggaatt | |
| | aggaaaaaacgtaaggctcaactgctctgctttgctgaatgaagaggatgtaatttattggatgttcggggaagaaaatg | |
| | gatcggatcctaatatacatgaagagaaagaaatgagaattatgactccagaaggcaaatggcatgcttcaaaagtatt | |
| | gagaattgaaaatattggtgaaagcaatctaaatgttttatataattgcactgtggccagcacgggaggcacagacacca | |
| | aaagcttcatcttggtgagaaaagcagacatggctgatatcccaggccacgtcttcacaagaggaatgatcatagctgtt | |
| | ttgatcttggtggcagtagtgtgcctagtgactgtgtgtgtcatttatagagttgacttggttctattttatagacatttaacga | |
| | gaagagatgaaacattaacagatggaaaaacatatgatgcttttgtgtcttacctaaaagaatgccgacctgaaaatgga | |
| | gaggagcacacctttgctgtggagattttgcccagggtgttggagaaacattttgggtataagttatgcatatttgaaagg | |
| | gatgtagtgcctggaggagctgttgttgatgaaatccactcactgatagagaaaagccgaagactaatcattgtcctaag | |
| | taaaagttatatgtctaatgaggtcaggtatgaacttgaaagtggactccatgaagcattggtggaaagaaaaattaaaat | |
| | aatcttaattgaatttacacctgttactgacttcacattcttgccccaatcactaaagcttttgaaatctcacagagttctgaag | |
| | tggaaggccgataaatctctttcttataactcaaggttctggaagaaccttctttacttaatgcctgcaaaaacagtcaagc | |
| | caggtagagacgaaccggaagtcttgcctgttctttccgagtcttgataa | |
| CAR 20A10N | atggccttaccagtgaccgccttgctcctgccgctggccttgctgctccacgccgccaggccgcaggtgcagctggtt | 122 |
| CD8-CD8-41BB | gaatctggcggcggacttgtgaagcctggcggatctctgagactgagctgtgccgccagcggcttcacctttagcacat | |
| CD3 6XIL2NFAT | acgccatgagctggatcagacaggcccctggcaaaggcctggaatgggtggcgtctattggcagagccggcagcac | |
| mCMV | ctactacagcgactctgtgaagggcagattcaccatcagccgggacaacgccaagaacagcctgtacctgcagatga | |
| IL18Ralpha | actccctgagagccgaggacaccgccgtgtactattgtgccagaggacccatctacaacgactacgacgagttcgcct | |
| | attggggccagggcacactggtcacagtttctagcggcggtggcggaagcggaggcggtggctccggtggcggag | |
| | gcagcgaaattgtgctgacacagagccccgccacactgtcactttctccaggcgaaagagccacactgagctgcaag | |
| | agcagccagagcgtgctgtactccagcaaccagaagaactacctggcctggtatcagcagaagcccggccaagctc | |
| | ctcggctgctgatctattgggccagcacaagagagagcggcatccctgccagattttctggcagcggctctggcaccg | |
| | atttcaccctgaccataagcagcctggaacctgaggacttcgccgtgtattactgccaccagtacctgagcagcctgac | |
| | ctttggcggaggcaccaaggtggaaatcaagcggacaacaacccctgcccccagacctcctaccccagcccctaca | |
| | attgccagccagcctctgagcctgaggcccgaggcttgtagacctgctgctggcggagccgtgcacaccagaggact | |
| | ggatttcgcctgcgacatctacatctgggcgcccttggccgggacttgtggggtccttctcctgtcactggttatcaccct | |
| | ttactgcaaacggggcagaaagaaactcctgtatatattcaaacaaccatttatgagaccagtacaaactactcaagagg | |
| | aagatggctgtagctgccgatttccagaagaagaagaaggaggatgtgaactgagagtgaagttcagcaggagcgc | |
| | agacgcccccgcgtacaagcagggccagaaccagctctataacgagctcaatctaggacgaagagaggagtacgat | |
| | gttttggacaagagacgtggccgggaccctgagatggggggaaagccgagaaggaagaaccctcaggaaggcctg | |
| | tacaatgaactgcagaaagataagatggcggaggcctacagtgagattgggatgaaaggcgagcgccggaggggc | |
| | aaggggcacgatggcctttaccagggtctcagtacagccaccaaggacacctacgacgcccttcacatgcaggccct | |
| | gccccctcgctgataagtttaaactgccagaacatttctctggcctaactggccggtaccggaggaaaaactgtttcata | |
| | cagaaggcgtggaggaaaaactgtttcatacagaaggcgtggaggaaaaactgtttcatacagaaggcgtggaggaa | |
| | aaactgtttcatacagaaggcgtggaggaaaaactgtttcatacagaaggcgtggaggaaaaactgtttcatacagaag | |
| | gcgtactagttaggcgtgtacggtgggaggcctatataagcagagctcgtttagtgaaccgtcagatcgcctggagac | |
| | gccatccacgctgttttgacctccatagaagacaccgggaccgatccagcctcgagagacccaatgctagccaccat | |
| | gaattgtagagaattacccttgaccctttgggtgcttatatctgtaagcactgcagaatcttgtacttcacgtccccacatta | |
| | ctgtggttgaaggggaacctttctatctgaaacattgctcgtgttcacttgcacatgagattgaaacaaccaccaaaagct | |
| | ggtacaaaagcagtggatcacaggaacatgtggagctgaacccaaggagttccagcagaattgctttgcatgattgtgt | |
| | tttggagttttggccagttgagttgaatgacacaggatcttactttttccaaatgaaaaattatactcagaaatggaaattaa | |
| | atgtcatcagaagaaataaacacagctgtttcactgaaagacaagtaactagtaaaattgtggaagttaaaaaattttttca | |
| | gataacctgtgaaaacagttactatcaaacactggtcaacagcacatcattgtataagaactgtaaaaagctactactgg | |
| | agaacaataaaaacccaacgataaagaagaacgccgagtttgaagatcaggggtattactcctgcgtgcatttccttcat | |
| | cataatggaaaactatttaatatcaccaaaaccttcaatataacaatagtggaagatcgcagtaatatagttccggttcttct | |
| | tggaccaaagcttaaccatgttgcagtggaattaggaaaaaacgtaaggctcaactgctctgctttgctgaatgaagag | |
| | gatgtaatttattggatgttcggggaagaaaatggatcggatcctaatatacatgaagagaaagaaatgagaattatgact | |
| | ccagaaggcaaatggcatgcttcaaaagtattgagaattgaaaatattggtgaaagcaatctaaatgttttatataattgca | |
| | ctgtggccagcacgggaggcacagacaccaaaagcttcatcttggtgagaaaagcagacatggctgatatcccaggc | |
| | cacgtcttcacaagaggaatgatcatagctgttttgatcttggtggcagtagtgtgcctagtgactgtgtgtgtcatttatag | |
| | agttgacttggttctattttatagacatttaacgagaagagatgaaacattaacagatggaaaaacatatgatgcttttgtgt | |
| | cttacctaaaagaatgccgacctgaaaatggagaggagcacacctttgctgtggagattttgcccagggtgttggagaa | |
| | acattttgggtataagttatgcatatttgaaagggatgtagtgcctggaggagctgttgttgatgaaatccactcactgata | |
| | gagaaaagccgaagactaatcattgtcctaagtaaaagttatatgtctaatgaggtcaggtatgaacttgaaagtggact | |
| | ccatgaagcattggtggaaagaaaaattaaaataatcttaattgaatttacacctgttactgacttcacattcttgccccaat | |
| | cactaaagcttttgaaatctcacagagttctgaagtggaaggccgataaatctctttcttataactcaaggttctggaagaa | |
| | ccttctttacttaatgcctgcaaaaacagtcaagccaggtagagacgaaccggaagtcttgcctgttctttccgagtcttg | |
| | ataa | |
| CAR 20A10N | atggccttaccagtgaccgccttgctcctgccgctggccttgctgctccacgccgccaggccgcaggtgcagctggtt | 123 |
| CD8-CD8-41BB | gaatctggcggcggacttgtgaagcctggcggatctctgagactgagctgtgccgccagcggcttcacctttagcacat | |
| CD3 3XIL2NFAT | acgccatgagctggatcagacaggcccctggcaaaggcctggaatgggtggcgtctattggcagagccggcagcac | |
| mCMV | ctactacagcgactctgtgaagggcagattcaccatcagccgggacaacgccaagaacagcctgtacctgcagatga | |
| IL18Ralpha | actccctgagagccgaggacaccgccgtgtactattgtgccagaggacccatctacaacgactacgacgagttcgcct | |
| | attggggccagggcacactggtcacagtttctagcggcggtggcggaagcggaggcggtggctccggtggcggag | |
| | gcagcgaaattgtgctgacacagagccccgccacactgtcactttctccaggcgaaagagccacactgagctgcaag | |
| | agcagccagagcgtgctgtactccagcaaccagaagaactacctggcctggtatcagcagaagcccggccaagctc | |
| | ctcggctgctgatctattgggccagcacaagagagagcggcatccctgccagattttctggcagcggctctggcaccg | |
| | atttcaccctgaccataagcagcctggaacctgaggacttcgccgtgtattactgccaccagtacctgagcagcctgac | |
| | ctttggcggaggcaccaaggtggaaatcaagcggacaacaacccctgcccccagacctcctaccccagcccctaca | |
| | attgccagccagcctctgagcctgaggcccgaggcttgtagacctgctgctggcggagccgtgcacaccagaggact | |
| | ggatttcgcctgcgacatctacatctgggcgcccttggccgggacttgtggggtccttctcctgtcactggttatcaccct | |
| | ttactgcaaacggggcagaaagaaactcctgtatatattcaaacaaccatttatgagaccagtacaaactactcaagagg | |
| | aagatggctgtagctgccgatttccagaagaagaagaaggaggatgtgaactgagagtgaagttcagcaggagcgc | |
| | agacgcccccgcgtacaagcagggccagaaccagctctataacgagctcaatctaggacgaagagaggagtacgat | |
| | gttttggacaagagacgtggccgggaccctgagatggggggaaagccgagaaggaagaaccctcaggaaggcctg | |
| | tacaatgaactgcagaaagataagatggcggaggcctacagtgagattgggatgaaaggcgagcgccggaggggc | |
| | aaggggcacgatggcctttaccagggtctcagtacagccaccaaggacacctacgacgcccttcacatgcaggccct | |
| | gccccctcgctgataagtttaaactgccagaacatttctctggcctaactggccggtaccggaggaaaaactgtttcata | |
| | cagaaggcgtggaggaaaaactgtttcatacagaaggcgtggaggaaaaactgtttcatacagaaggcgtactagtta | |
| | ggcgtgtacggtgggaggcctatataagcagagctcgtttagtgaaccgtcagatcgcctggagacgccatccacgct | |
| | gttttgacctccatagaagacaccgggaccgatccagcctcgagagacccaatgctagccaccatgaattgtagaga | |
| | attacccttgaccctttgggtgcttatatctgtaagcactgcagaatcttgtacttcacgtccccacattactgtggttgaag | |
| | gggaacctttctatctgaaacattgctcgtgttcacttgcacatgagattgaaacaaccaccaaaagctggtacaaaagc | |
| | agtggatcacaggaacatgtggagctgaacccaaggagttccagcagaattgctttgcatgattgtgttttggagttttgg | |
| | ccagttgagttgaatgacacaggatcttactttttccaaatgaaaaattatactcagaaatggaaattaaatgtcatcagaa | |
| | gaaataaacacagctgtttcactgaaagacaagtaactagtaaaattgtggaagttaaaaaattttttcagataacctgtga | |
| | aaacagttactatcaaacactggtcaacagcacatcattgtataagaactgtaaaaagctactactggagaacaataaaa | |
| | acccaacgataaagaagaacgccgagtttgaagatcaggggtattactcctgcgtgcatttccttcatcataatggaaaa | |
| | ctatttaatatcaccaaaaccttcaatataacaatagtggaagatcgcagtaatatagttccggttcttcttggaccaaagct | |
| | taaccatgttgcagtggaattaggaaaaaacgtaaggctcaactgctctgctttgctgaatgaagaggatgtaatttattg | |
| | gatgttcggggaagaaaatggatcggatcctaatatacatgaagagaaagaaatgagaattatgactccagaaggcaa | |
| | atggcatgcttcaaaagtattgagaattgaaaatattggtgaaagcaatctaaatgttttatataattgcactgtggccagca | |
| | cgggaggcacagacaccaaaagcttcatcttggtgagaaaagcagacatggctgatatcccaggccacgtcttcaca | |
| | agaggaatgatcatagctgttttgatcttggtggcagtagtgtgcctagtgactgtgtgtgtcatttatagagttgacttggtt | |
| | ctattttatagacatttaacgagaagagatgaaacattaacagatggaaaaacatatgatgcttttgtgtcttacctaaaaga | |
| | atgccgacctgaaaatggagaggagcacacctttgctgtggagattttgcccagggtgttggagaaacattttgggtata | |
| | agttatgcatatttgaaagggatgtagtgcctggaggagctgttgttgatgaaatccactcactgatagagaaaagccga | |
| | agactaatcattgtcctaagtaaaagttatatgtctaatgaggtcaggtatgaacttgaaagtggactccatgaagcattgg | |
| | tggaaagaaaaattaaaataatcttaattgaatttacacctgttactgacttcacattcttgccccaatcactaaagcttttga | |
| | aatctcacagagttctgaagtggaaggccgataaatctctttcttataactcaaggttctggaagaaccttctttacttaatg | |
| | cctgcaaaaacagtcaagccaggtagagacgaaccggaagtcttgcctgttctttccgagtcttgataa | |
| CAR 20A10N | atggccttaccagtgaccgccttgctcctgccgctggccttgctgctccacgccgccaggccgcaggtgcagctggtt | 124 |
| CD28-CD28-CD28 | gaatctggcggcggacttgtgaagcctggcggatctctgagactgagctgtgccgccagcggcttcacctttagcacat | |
| CD3 | acgccatgagctggatcagacaggcccctggcaaaggcctggaatgggtggcgtctattggcagagccggcagcac | |
| 6XFOXP3NFAT | ctactacagcgactctgtgaagggcagattcaccatcagccgggacaacgccaagaacagcctgtacctgcagatga | |
| mCMV | actccctgagagccgaggacaccgccgtgtactattgtgccagaggacccatctacaacgactacgacgagttcgcct | |
| IL18Ralpha | attggggccagggcacactggtcacagtttctagcggcggtggcggaagcggaggcggtggctccggtggcggag | |
| | gcagcgaaattgtgctgacacagagccccgccacactgtcactttctccaggcgaaagagccacactgagctgcaag | |
| | agcagccagagcgtgctgtactccagcaaccagaagaactacctggcctggtatcagcagaagcccggccaagctc | |
| | ctcggctgctgatctattgggccagcacaagagagagcggcatccctgccagattttctggcagcggctctggcaccg | |
| | atttcaccctgaccataagcagcctggaacctgaggacttcgccgtgtattactgccaccagtacctgagcagcctgac | |
| | ctttggcggaggcaccaaggtggaaatcaagcggaagcacctgtgtccttctccactgttccccggccctagcaagcc | |
| | tttctgggtcctggtggtggtgggcggagtgctggcctgctacagcctgctggtgaccgtggcctttatcatcttctgggt | |
| | gaggagtaagaggagcaggctcctgcacagtgactacatgaacatgactcctagaagacctgggcctaccagaaag | |
| | cattaccagccctatgccccaccacgcgacttcgcagcctatcgctccagagtgaagttcagcaggagcgcagacgc | |
| | ccccgcgtacaagcagggccagaaccagctctataacgagctcaatctaggacgaagagaggagtacgatgttttgg | |
| | acaagagacgtggccgggaccctgagatggggggaaagccgagaaggaagaaccctcaggaaggcctgtacaat | |
| | gaactgcagaaagataagatggcggaggcctacagtgagattgggatgaaaggcgagcgccggaggggcaaggg | |
| | gcacgatggcctttaccagggtctcagtacagccaccaaggacacctacgacgcccttcacatgcaggccctgcccc | |
| | ctcgctgataagtttaaactgccagaacatttctctggcctaactggccggtaccggcttcattttttccatttactgcagag | |
| | gcttcattttttccatttactgcagaggcttcattttttccatttactgcagaggcttcattttttccatttactgcagaggcttcat | |
| | tttttccatttactgcagaggcttcattttttccatttactgcagaactagttaggcgtgtacggtgggaggcctatataagca | |
| | gagctcgtttagtgaaccgtcagatcgcctggagacgccatccacgctgttttgacctccatagaagacaccgggacc | |
| | gatccagcctcgagagacccaatgctagccaccatgaattgtagagaattacccttgaccctttgggtgcttatatctgt | |
| | aagcactgcagaatcttgtacttcacgtccccacattactgtggttgaaggggaacctttctatctgaaacattgctcgtgtt | |
| | cacttgcacatgagattgaaacaaccaccaaaagctggtacaaaagcagtggatcacaggaacatgtggagctgaac | |
| | ccaaggagttccagcagaattgctttgcatgattgtgttttggagttttggccagttgagttgaatgacacaggatcttactt | |
| | tttccaaatgaaaaattatactcagaaatggaaattaaatgtcatcagaagaaataaacacagctgtttcactgaaagaca | |
| | agtaactagtaaaattgtggaagttaaaaaattttttcagataacctgtgaaaacagttactatcaaacactggtcaacagc | |
| | acatcattgtataagaactgtaaaaagctactactggagaacaataaaaacccaacgataaagaagaacgccgagtttg | |
| | aagatcaggggtattactcctgcgtgcatttccttcatcataatggaaaactatttaatatcaccaaaaccttcaatataaca | |
| | atagtggaagatcgcagtaatatagttccggttcttcttggaccaaagcttaaccatgttgcagtggaattaggaaaaaac | |
| | gtaaggctcaactgctctgctttgctgaatgaagaggatgtaatttattggatgttcggggaagaaaatggatcggatcct | |
| | aatatacatgaagagaaagaaatgagaattatgactccagaaggcaaatggcatgcttcaaaagtattgagaattgaaa | |
| | atattggtgaaagcaatctaaatgttttatataattgcactgtggccagcacgggaggcacagacaccaaaagcttcatct | |
| | tggtgagaaaagcagacatggctgatatcccaggccacgtcttcacaagaggaatgatcatagctgttttgatcttggtg | |
| | gcagtagtgtgcctagtgactgtgtgtgtcatttatagagttgacttggttctattttatagacatttaacgagaagagatga | |
| | aacattaacagatggaaaaacatatgatgcttttgtgtcttacctaaaagaatgccgacctgaaaatggagaggagcaca | |
| | cctttgctgtggagattttgcccagggtgttggagaaacattttgggtataagttatgcatatttgaaagggatgtagtgcct | |
| | ggaggagctgttgttgatgaaatccactcactgatagagaaaagccgaagactaatcattgtcctaagtaaaagttatat | |
| | gtctaatgaggtcaggtatgaacttgaaagtggactccatgaagcattggtggaaagaaaaattaaaataatcttaattga | |
| | atttacacctgttactgacttcacattcttgccccaatcactaaagcttttgaaatctcacagagttctgaagtggaaggccg | |
| | ataaatctctttcttataactcaaggttctggaagaaccttctttacttaatgcctgcaaaaacagtcaagccaggtagaga | |
| | cgaaccggaagtcttgcctgttctttccgagtcttgataa | |
| CAR 20A10N | atggccttaccagtgaccgccttgctcctgccgctggccttgctgctccacgccgccaggccgcaggtgcagctggtt | 125 |
| CD28-CD28-CD28 | gaatctggcggcggacttgtgaagcctggcggatctctgagactgagctgtgccgccagcggcttcacctttagcacat | |
| CD3 | acgccatgagctggatcagacaggcccctggcaaaggcctggaatgggtggcgtctattggcagagccggcagcac | |
| 3XFOXP3NFAT | ctactacagcgactctgtgaagggcagattcaccatcagccgggacaacgccaagaacagcctgtacctgcagatga | |
| mCMV | actccctgagagccgaggacaccgccgtgtactattgtgccagaggacccatctacaacgactacgacgagttcgcct | |
| IL18Ralpha | attggggccagggcacactggtcacagtttctagcggcggtggcggaagcggaggcggtggctccggtggcggag | |
| | gcagcgaaattgtgctgacacagagccccgccacactgtcactttctccaggcgaaagagccacactgagctgcaag | |
| | agcagccagagcgtgctgtactccagcaaccagaagaactacctggcctggtatcagcagaagcccggccaagctc | |
| | ctcggctgctgatctattgggccagcacaagagagagcggcatccctgccagattttctggcagcggctctggcaccg | |
| | atttcaccctgaccataagcagcctggaacctgaggacttcgccgtgtattactgccaccagtacctgagcagcctgac | |
| | ctttggcggaggcaccaaggtggaaatcaagcggaagcacctgtgtccttctccactgttccccggccctagcaagcc | |
| | tttctgggtcctggtggtggtgggcggagtgctggcctgctacagcctgctggtgaccgtggcctttatcatcttctgggt | |
| | gaggagtaagaggagcaggctcctgcacagtgactacatgaacatgactcctagaagacctgggcctaccagaaag | |
| | cattaccagccctatgccccaccacgcgacttcgcagcctatcgctccagagtgaagttcagcaggagcgcagacgc | |
| | ccccgcgtacaagcagggccagaaccagctctataacgagctcaatctaggacgaagagaggagtacgatgttttgg | |
| | acaagagacgtggccgggaccctgagatggggggaaagccgagaaggaagaaccctcaggaaggcctgtacaat | |
| | gaactgcagaaagataagatggcggaggcctacagtgagattgggatgaaaggcgagcgccggaggggcaaggg | |
| | gcacgatggcctttaccagggtctcagtacagccaccaaggacacctacgacgcccttcacatgcaggccctgcccc | |
| | ctcgctgataagtttaaactgccagaacatttctctggcctaactggccggtaccggcttcattttttccatttactgcagag | |
| | gcttcattttttccatttactgcagaggcttcattttttccatttactgcagaactagttaggcgtgtacggtgggaggcctat | |
| | ataagcagagctcgtttagtgaaccgtcagatcgcctggagacgccatccacgctgttttgacctccatagaagacacc | |
| | gggaccgatccagcctcgagagacccaatgctagccaccatgaattgtagagaattacccttgaccctttgggtgctta | |
| | tatctgtaagcactgcagaatcttgtacttcacgtccccacattactgtggttgaaggggaacctttctatctgaaacattgc | |
| | tcgtgttcacttgcacatgagattgaaacaaccaccaaaagctggtacaaaagcagtggatcacaggaacatgtggag | |
| | ctgaacccaaggagttccagcagaattgctttgcatgattgtgttttggagttttggccagttgagttgaatgacacaggat | |
| | cttactttttccaaatgaaaaattatactcagaaatggaaattaaatgtcatcagaagaaataaacacagctgtttcactgaa | |
| | agacaagtaactagtaaaattgtggaagttaaaaaattttttcagataacctgtgaaaacagttactatcaaacactggtca | |
| | acagcacatcattgtataagaactgtaaaaagctactactggagaacaataaaaacccaacgataaagaagaacgccg | |
| | agtttgaagatcaggggtattactcctgcgtgcatttccttcatcataatggaaaactatttaatatcaccaaaaccttcaata | |
| | taacaatagtggaagatcgcagtaatatagttccggttcttcttggaccaaagcttaaccatgttgcagtggaattaggaa | |
| | aaaacgtaaggctcaactgctctgctttgctgaatgaagaggatgtaatttattggatgttcggggaagaaaatggatcg | |
| | gatcctaatatacatgaagagaaagaaatgagaattatgactccagaaggcaaatggcatgcttcaaaagtattgagaat | |
| | tgaaaatattggtgaaagcaatctaaatgttttatataattgcactgtggccagcacgggaggcacagacaccaaaagct | |
| | tcatcttggtgagaaaagcagacatggctgatatcccaggccacgtcttcacaagaggaatgatcatagctgttttgatct | |
| | tggtggcagtagtgtgcctagtgactgtgtgtgtcatttatagagttgacttggttctattttatagacatttaacgagaaga | |
| | gatgaaacattaacagatggaaaaacatatgatgcttttgtgtcttacctaaaagaatgccgacctgaaaatggagagga | |
| | gcacacctttgctgtggagattttgcccagggtgttggagaaacattttgggtataagttatgcatatttgaaagggatgta | |
| | gtgcctggaggagctgttgttgatgaaatccactcactgatagagaaaagccgaagactaatcattgtcctaagtaaaag | |
| | ttatatgtctaatgaggtcaggtatgaacttgaaagtggactccatgaagcattggtggaaagaaaaattaaaataatctta | |
| | attgaatttacacctgttactgacttcacattcttgccccaatcactaaagcttttgaaatctcacagagttctgaagtggaa | |
| | ggccgataaatctctttcttataactcaaggttctggaagaaccttctttacttaatgcctgcaaaaacagtcaagccaggt | |
| | agagacgaaccggaagtcttgcctgttctttccgagtcttgataa | |
| CAR 20A10N | atggccttaccagtgaccgccttgctcctgccgctggccttgctgctccacgccgccaggccgcaggtgcagctggtt | 126 |
| CD28-CD28-CD28 | gaatctggcggcggacttgtgaagcctggcggatctctgagactgagctgtgccgccagcggcttcacctttagcacat | |
| CD3 6XIL2NFAT | acgccatgagctggatcagacaggcccctggcaaaggcctggaatgggtggcgtctattggcagagccggcagcac | |
| mCMV | ctactacagcgactctgtgaagggcagattcaccatcagccgggacaacgccaagaacagcctgtacctgcagatga | |
| IL18Ralpha | actccctgagagccgaggacaccgccgtgtactattgtgccagaggacccatctacaacgactacgacgagttcgcct | |
| | attggggccagggcacactggtcacagtttctagcggcggtggcggaagcggaggcggtggctccggtggcggag | |
| | gcagcgaaattgtgctgacacagagccccgccacactgtcactttctccaggcgaaagagccacactgagctgcaag | |
| | agcagccagagcgtgctgtactccagcaaccagaagaactacctggcctggtatcagcagaagcccggccaagctc | |
| | ctcggctgctgatctattgggccagcacaagagagagcggcatccctgccagattttctggcagcggctctggcaccg | |
| | atttcaccctgaccataagcagcctggaacctgaggacttcgccgtgtattactgccaccagtacctgagcagcctgac | |
| | ctttggcggaggcaccaaggtggaaatcaagcggaagcacctgtgtccttctccactgttccccggccctagcaagcc | |
| | tttctgggtcctggtggtggtgggcggagtgctggcctgctacagcctgctggtgaccgtggcctttatcatcttctgggt | |
| | gaggagtaagaggagcaggctcctgcacagtgactacatgaacatgactcctagaagacctgggcctaccagaaag | |
| | cattaccagccctatgccccaccacgcgacttcgcagcctatcgctccagagtgaagttcagcaggagcgcagacgc | |
| | ccccgcgtacaagcagggccagaaccagctctataacgagctcaatctaggacgaagagaggagtacgatgttttgg | |
| | acaagagacgtggccgggaccctgagatggggggaaagccgagaaggaagaaccctcaggaaggcctgtacaat | |
| | gaactgcagaaagataagatggcggaggcctacagtgagattgggatgaaaggcgagcgccggaggggcaaggg | |
| | gcacgatggcctttaccagggtctcagtacagccaccaaggacacctacgacgcccttcacatgcaggccctgcccc | |
| | ctcgctgataagtttaaactgccagaacatttctctggcctaactggccggtaccggaggaaaaactgtttcatacagaa | |
| | ggcgtggaggaaaaactgtttcatacagaaggcgtggaggaaaaactgtttcatacagaaggcgtggaggaaaaact | |
| | gtttcatacagaaggcgtggaggaaaaactgtttcatacagaaggcgtggaggaaaaactgtttcatacagaaggcgt | |
| | actagttaggcgtgtacggtgggaggcctatataagcagagctcgtttagtgaaccgtcagatcgcctggagacgccat | |
| | ccacgctgttttgacctccatagaagacaccgggaccgatccagcctcgagagacccaatgctagccaccatgaatt | |
| | gtagagaattacccttgaccctttgggtgcttatatctgtaagcactgcagaatcttgtacttcacgtccccacattactgtg | |
| | gttgaaggggaacctttctatctgaaacattgctcgtgttcacttgcacatgagattgaaacaaccaccaaaagctggtac | |
| | aaaagcagtggatcacaggaacatgtggagctgaacccaaggagttccagcagaattgctttgcatgattgtgttttgga | |
| | gttttggccagttgagttgaatgacacaggatcttactttttccaaatgaaaaattatactcagaaatggaaattaaatgtcat | |
| | cagaagaaataaacacagctgtttcactgaaagacaagtaactagtaaaattgtggaagttaaaaaattttttcagataac | |
| | ctgtgaaaacagttactatcaaacactggtcaacagcacatcattgtataagaactgtaaaaagctactactggagaaca | |
| | ataaaaacccaacgataaagaagaacgccgagtttgaagatcaggggtattactcctgcgtgcatttccttcatcataat | |
| | ggaaaactatttaatatcaccaaaaccttcaatataacaatagtggaagatcgcagtaatatagttccggttcttcttggac | |
| | caaagcttaaccatgttgcagtggaattaggaaaaaacgtaaggctcaactgctctgctttgctgaatgaagaggatgta | |
| | atttattggatgttcggggaagaaaatggatcggatcctaatatacatgaagagaaagaaatgagaattatgactccaga | |
| | aggcaaatggcatgcttcaaaagtattgagaattgaaaatattggtgaaagcaatctaaatgttttatataattgcactgtg | |
| | gccagcacgggaggcacagacaccaaaagcttcatcttggtgagaaaagcagacatggctgatatcccaggccacg | |
| | tcttcacaagaggaatgatcatagctgttttgatcttggtggcagtagtgtgcctagtgactgtgtgtgtcatttatagagtt | |
| | gacttggttctattttatagacatttaacgagaagagatgaaacattaacagatggaaaaacatatgatgcttttgtgtctta | |
| | cctaaaagaatgccgacctgaaaatggagaggagcacacctttgctgtggagattttgcccagggtgttggagaaaca | |
| | ttttgggtataagttatgcatatttgaaagggatgtagtgcctggaggagctgttgttgatgaaatccactcactgatagag | |
| | aaaagccgaagactaatcattgtcctaagtaaaagttatatgtctaatgaggtcaggtatgaacttgaaagtggactccat | |
| | gaagcattggtggaaagaaaaattaaaataatcttaattgaatttacacctgttactgacttcacattcttgccccaatcact | |
| | aaagcttttgaaatctcacagagttctgaagtggaaggccgataaatctctttcttataactcaaggttctggaagaacctt | |
| | ctttacttaatgcctgcaaaaacagtcaagccaggtagagacgaaccggaagtcttgcctgttctttccgagtcttgataa | |
| CAR 20A10N | atggccttaccagtgaccgccttgctcctgccgctggccttgctgctccacgccgccaggccgcaggtgcagctggtt | 127 |
| CD28-CD28-CD28 | gaatctggcggcggacttgtgaagcctggcggatctctgagactgagctgtgccgccagcggcttcacctttagcacat | |
| CD3 3XIL2NFAT | acgccatgagctggatcagacaggcccctggcaaaggcctggaatgggtggcgtctattggcagagccggcagcac | |
| mCMV | ctactacagcgactctgtgaagggcagattcaccatcagccgggacaacgccaagaacagcctgtacctgcagatga | |
| IL18Ralpha | actccctgagagccgaggacaccgccgtgtactattgtgccagaggacccatctacaacgactacgacgagttcgcct | |
| | attggggccagggcacactggtcacagtttctagcggcggtggcggaagcggaggcggtggctccggtggcggag | |
| | gcagcgaaattgtgctgacacagagccccgccacactgtcactttctccaggcgaaagagccacactgagctgcaag | |
| | agcagccagagcgtgctgtactccagcaaccagaagaactacctggcctggtatcagcagaagcccggccaagctc | |
| | ctcggctgctgatctattgggccagcacaagagagagcggcatccctgccagattttctggcagcggctctggcaccg | |
| | atttcaccctgaccataagcagcctggaacctgaggacttcgccgtgtattactgccaccagtacctgagcagcctgac | |
| | ctttggcggaggcaccaaggtggaaatcaagcggaagcacctgtgtccttctccactgttccccggccctagcaagcc | |
| | tttctgggtcctggtggtggtgggcggagtgctggcctgctacagcctgctggtgaccgtggcctttatcatcttctgggt | |
| | gaggagtaagaggagcaggctcctgcacagtgactacatgaacatgactcctagaagacctgggcctaccagaaag | |
| | cattaccagccctatgccccaccacgcgacttcgcagcctatcgctccagagtgaagttcagcaggagcgcagacgc | |
| | ccccgcgtacaagcagggccagaaccagctctataacgagctcaatctaggacgaagagaggagtacgatgttttgg | |
| | acaagagacgtggccgggaccctgagatggggggaaagccgagaaggaagaaccctcaggaaggcctgtacaat | |
| | gaactgcagaaagataagatggcggaggcctacagtgagattgggatgaaaggcgagcgccggaggggcaaggg | |
| | gcacgatggcctttaccagggtctcagtacagccaccaaggacacctacgacgcccttcacatgcaggccctgcccc | |
| | ctcgctgataagtttaaactgccagaacatttctctggcctaactggccggtaccggaggaaaaactgtttcatacagaa | |
| | ggcgtggaggaaaaactgtttcatacagaaggcgtggaggaaaaactgtttcatacagaaggcgtactagttaggcgt | |
| | gtacggtgggaggcctatataagcagagctcgtttagtgaaccgtcagatcgcctggagacgccatccacgctgttttg | |
| | acctccatagaagacaccgggaccgatccagcctcgagagacccaatgctagccaccatgaattgtagagaattacc | |
| | cttgaccctttgggtgcttatatctgtaagcactgcagaatcttgtacttcacgtccccacattactgtggttgaaggggaa | |
| | cctttctatctgaaacattgctcgtgttcacttgcacatgagattgaaacaaccaccaaaagctggtacaaaagcagtgga | |
| | tcacaggaacatgtggagctgaacccaaggagttccagcagaattgctttgcatgattgtgttttggagttttggccagtt | |
| | gagttgaatgacacaggatcttactttttccaaatgaaaaattatactcagaaatggaaattaaatgtcatcagaagaaata | |
| | aacacagctgtttcactgaaagacaagtaactagtaaaattgtggaagttaaaaaattttttcagataacctgtgaaaaca | |
| | gttactatcaaacactggtcaacagcacatcattgtataagaactgtaaaaagctactactggagaacaataaaaaccca | |
| | acgataaagaagaacgccgagtttgaagatcaggggtattactcctgcgtgcatttccttcatcataatggaaaactattt | |
| | aatatcaccaaaaccttcaatataacaatagtggaagatcgcagtaatatagttccggttcttcttggaccaaagcttaacc | |
| | atgttgcagtggaattaggaaaaaacgtaaggctcaactgctctgctttgctgaatgaagaggatgtaatttattggatgtt | |
| | cggggaagaaaatggatcggatcctaatatacatgaagagaaagaaatgagaattatgactccagaaggcaaatggc | |
| | atgcttcaaaagtattgagaattgaaaatattggtgaaagcaatctaaatgttttatataattgcactgtggccagcacggg | |
| | aggcacagacaccaaaagcttcatcttggtgagaaaagcagacatggctgatatcccaggccacgtcttcacaagag | |
| | gaatgatcatagctgttttgatcttggtggcagtagtgtgcctagtgactgtgtgtgtcatttatagagttgacttggttctatt | |
| | ttatagacatttaacgagaagagatgaaacattaacagatggaaaaacatatgatgcttttgtgtcttacctaaaagaatgc | |
| | cgacctgaaaatggagaggagcacacctttgctgtggagattttgcccagggtgttggagaaacattttgggtataagtt | |
| | atgcatatttgaaagggatgtagtgcctggaggagctgttgttgatgaaatccactcactgatagagaaaagccgaaga | |
| | ctaatcattgtcctaagtaaaagttatatgtctaatgaggtcaggtatgaacttgaaagtggactccatgaagcattggtgg | |
| | aaagaaaaattaaaataatcttaattgaatttacacctgttactgacttcacattcttgccccaatcactaaagcttttgaaatc | |
| | tcacagagttctgaagtggaaggccgataaatctctttcttataactcaaggttctggaagaaccttctttacttaatgcctg | |
| | caaaaacagtcaagccaggtagagacgaaccggaagtcttgcctgttctttccgagtcttgataa | |
| CAR 20A10N | atggccttaccagtgaccgccttgctcctgccgctggccttgctgctccacgccgccaggccgcaggtgcagctggtt | 128 |
| CD28-CD28-CD28 | gaatctggcggcggacttgtgaagcctggcggatctctgagactgagctgtgccgccagcggcttcacctttagcacat | |
| 1XX | acgccatgagctggatcagacaggcccctggcaaaggcctggaatgggtggcgtctattggcagagccggcagcac | |
| 6XFOXP3NFAT | ctactacagcgactctgtgaagggcagattcaccatcagccgggacaacgccaagaacagcctgtacctgcagatga | |
| mCMV | actccctgagagccgaggacaccgccgtgtactattgtgccagaggacccatctacaacgactacgacgagttcgcct | |
| IL18Ralpha | attggggccagggcacactggtcacagtttctagcggcggtggcggaagcggaggcggtggctccggtggcggag | |
| | gcagcgaaattgtgctgacacagagccccgccacactgtcactttctccaggcgaaagagccacactgagctgcaag | |
| | agcagccagagcgtgctgtactccagcaaccagaagaactacctggcctggtatcagcagaagcccggccaagctc | |
| | ctcggctgctgatctattgggccagcacaagagagagcggcatccctgccagattttctggcagcggctctggcaccg | |
| | atttcaccctgaccataagcagcctggaacctgaggacttcgccgtgtattactgccaccagtacctgagcagcctgac | |
| | ctttggcggaggcaccaaggtggaaatcaagcggaagcacctgtgtccttctccactgttccccggccctagcaagcc | |
| | tttctgggtcctggtggtggtgggcggagtgctggcctgctacagcctgctggtgaccgtggcctttatcatcttctgggt | |
| | gaggagtaagaggagcaggctcctgcacagtgactacatgaacatgactcctagaagacctgggcctaccagaaag | |
| | cattaccagccctatgccccaccacgcgacttcgcagcctatcgctccagagtgaagttcagcaggagcgcagacgc | |
| | ccccgcgtacaagcagggccagaaccagctctataacgagctcaatctaggacgaagagaggagtacgatgttttgg | |
| | acaagagacgtggccgggaccctgagatggggggaaagccgagaaggaagaaccctcaggaaggcctgttcaat | |
| | gaactgcagaaagataagatggcggaggccttcagtgagattgggatgaaaggcgagcgccggaggggcaaggg | |
| | gcacgatggccttttccagggtctcagtacagccaccaaggacaccttcgacgcccttcacatgcaggccctgccccc | |
| | tcgctgataagtttaaactgccagaacattactctggcctaactggccggtaccggcttcattttttccatttactgcagagg | |
| | cttcattttttccatttactgcagaggcttcattttttccatttactgcagaggcttcattttttccatttactgcagaggcttcattt | |
| | tttccatttactgcagaggcttcattttttccatttactgcagaactagttaggcgtgtacggtgggaggcctatataagcag | |
| | agctcgtttagtgaaccgtcagatcgcctggagacgccatccacgctgttttgacctccatagaagacaccgggaccg | |
| | atccagcctcgagagacccaatgctagccaccatgaattgtagagaattacccttgaccctttgggtgcttatatctgtaa | |
| | gcactgcagaatcttgtacttcacgtccccacattactgtggttgaaggggaacctttctatctgaaacattgctcgtgttca | |
| | cttgcacatgagattgaaacaaccaccaaaagctggtacaaaagcagtggatcacaggaacatgtggagctgaaccc | |
| | aaggagttccagcagaattgctttgcatgattgtgttttggagttttggccagttgagttgaatgacacaggatcttacttttt | |
| | ccaaatgaaaaattatactcagaaatggaaattaaatgtcatcagaagaaataaacacagctgtttcactgaaagacaag | |
| | taactagtaaaattgtggaagttaaaaaattttttcagataacctgtgaaaacagttactatcaaacactggtcaacagcac | |
| | atcattgtataagaactgtaaaaagctactactggagaacaataaaaacccaacgataaagaagaacgccgagtttgaa | |
| | gatcaggggtattactcctgcgtgcatttccttcatcataatggaaaactatttaatatcaccaaaaccttcaatataacaat | |
| | agtggaagatcgcagtaatatagttccggttcttcttggaccaaagcttaaccatgttgcagtggaattaggaaaaaacgt | |
| | aaggctcaactgctctgctttgctgaatgaagaggatgtaatttattggatgttcggggaagaaaatggatcggatcctaa | |
| | tatacatgaagagaaagaaatgagaattatgactccagaaggcaaatggcatgcttcaaaagtattgagaattgaaaata | |
| | ttggtgaaagcaatctaaatgttttatataattgcactgtggccagcacgggaggcacagacaccaaaagcttcatcttg | |
| | gtgagaaaagcagacatggctgatatcccaggccacgtcttcacaagaggaatgatcatagctgttttgatcttggtggc | |
| | agtagtgtgcctagtgactgtgtgtgtcatttatagagttgacttggttctattttatagacatttaacgagaagagatgaaa | |
| | cattaacagatggaaaaacatatgatgcttttgtgtcttacctaaaagaatgccgacctgaaaatggagaggagcacacc | |
| | tttgctgtggagattttgcccagggtgttggagaaacattttgggtataagttatgcatatttgaaagggatgtagtgcctg | |
| | gaggagctgttgttgatgaaatccactcactgatagagaaaagccgaagactaatcattgtcctaagtaaaagttatatgt | |
| | ctaatgaggtcaggtatgaacttgaaagtggactccatgaagcattggtggaaagaaaaattaaaataatcttaattgaat | |
| | ttacacctgttactgacttcacattcttgccccaatcactaaagcttttgaaatctcacagagttctgaagtggaaggccgat | |
| | aaatctctttcttataactcaaggttctggaagaaccttctttacttaatgcctgcaaaaacagtcaagccaggtagagacg | |
| | aaccggaagtcttgcctgttctttccgagtcttgataa | |
| CAR 20A10N | atggccttaccagtgaccgccttgctcctgccgctggccttgctgctccacgccgccaggccgcaggtgcagctggtt | 129 |
| CD28-CD28-CD28 | gaatctggcggcggacttgtgaagcctggcggatctctgagactgagctgtgccgccagcggcttcacctttagcacat | |
| 1XX | acgccatgagctggatcagacaggcccctggcaaaggcctggaatgggtggcgtctattggcagagccggcagcac | |
| 3XFOXP3NFAT | ctactacagcgactctgtgaagggcagattcaccatcagccgggacaacgccaagaacagcctgtacctgcagatga | |
| mCMV | actccctgagagccgaggacaccgccgtgtactattgtgccagaggacccatctacaacgactacgacgagttcgcct | |
| IL18Ralpha | attggggccagggcacactggtcacagtttctagcggcggtggcggaagcggaggcggtggctccggtggcggag | |
| | gcagcgaaattgtgctgacacagagccccgccacactgtcactttctccaggcgaaagagccacactgagctgcaag | |
| | agcagccagagcgtgctgtactccagcaaccagaagaactacctggcctggtatcagcagaagcccggccaagctc | |
| | ctcggctgctgatctattgggccagcacaagagagagcggcatccctgccagattttctggcagcggctctggcaccg | |
| | atttcaccctgaccataagcagcctggaacctgaggacttcgccgtgtattactgccaccagtacctgagcagcctgac | |
| | ctttggcggaggcaccaaggtggaaatcaagcggaagcacctgtgtccttctccactgttccccggccctagcaagcc | |
| | tttctgggtcctggtggtggtgggcggagtgctggcctgctacagcctgctggtgaccgtggcctttatcatcttctgggt | |
| | gaggagtaagaggagcaggctcctgcacagtgactacatgaacatgactcctagaagacctgggcctaccagaaag | |
| | cattaccagccctatgccccaccacgcgacttcgcagcctatcgctccagagtgaagttcagcaggagcgcagacgc | |
| | ccccgcgtacaagcagggccagaaccagctctataacgagctcaatctaggacgaagagaggagtacgatgttttgg | |
| | acaagagacgtggccgggaccctgagatggggggaaagccgagaaggaagaaccctcaggaaggcctgttcaat | |
| | gaactgcagaaagataagatggcggaggccttcagtgagattgggatgaaaggcgagcgccggaggggcaaggg | |
| | gcacgatggccttttccagggtctcagtacagccaccaaggacaccttcgacgcccttcacatgcaggccctgccccc | |
| | tcgctgataagtttaaactgccagaacattactctggcctaactggccggtaccggcttcattttttccatttactgcagagg | |
| | cttcattttttccatttactgcagaggcttcattttttccatttactgcagaactagttaggcgtgtacggtgggaggcctatat | |
| | aagcagagctcgtttagtgaaccgtcagatcgcctggagacgccatccacgctgttttgacctccatagaagacaccg | |
| | ggaccgatccagcctcgagagacccaatgctagccaccatgaattgtagagaattacccttgaccctttgggtgcttat | |
| | atctgtaagcactgcagaatcttgtacttcacgtccccacattactgtggttgaaggggaacctttctatctgaaacattgct | |
| | cgtgttcacttgcacatgagattgaaacaaccaccaaaagctggtacaaaagcagtggatcacaggaacatgtggagc | |
| | tgaacccaaggagttccagcagaattgctttgcatgattgtgttttggagttttggccagttgagttgaatgacacaggatc | |
| | ttactttttccaaatgaaaaattatactcagaaatggaaattaaatgtcatcagaagaaataaacacagctgtttcactgaaa | |
| | gacaagtaactagtaaaattgtggaagttaaaaaattttttcagataacctgtgaaaacagttactatcaaacactggtcaa | |
| | cagcacatcattgtataagaactgtaaaaagctactactggagaacaataaaaacccaacgataaagaagaacgccga | |
| | gtttgaagatcaggggtattactcctgcgtgcatttccttcatcataatggaaaactatttaatatcaccaaaaccttcaatat | |
| | aacaatagtggaagatcgcagtaatatagttccggttcttcttggaccaaagcttaaccatgttgcagtggaattaggaaa | |
| | aaacgtaaggctcaactgctctgctttgctgaatgaagaggatgtaatttattggatgttcggggaagaaaatggatcgg | |
| | atcctaatatacatgaagagaaagaaatgagaattatgactccagaaggcaaatggcatgcttcaaaagtattgagaatt | |
| | gaaaatattggtgaaagcaatctaaatgttttatataattgcactgtggccagcacgggaggcacagacaccaaaagctt | |
| | catcttggtgagaaaagcagacatggctgatatcccaggccacgtcttcacaagaggaatgatcatagctgttttgatctt | |
| | ggtggcagtagtgtgcctagtgactgtgtgtgtcatttatagagttgacttggttctattttatagacatttaacgagaagag | |
| | atgaaacattaacagatggaaaaacatatgatgcttttgtgtcttacctaaaagaatgccgacctgaaaatggagaggag | |
| | cacacctttgctgtggagattttgcccagggtgttggagaaacattttgggtataagttatgcatatttgaaagggatgtag | |
| | tgcctggaggagctgttgttgatgaaatccactcactgatagagaaaagccgaagactaatcattgtcctaagtaaaagt | |
| | tatatgtctaatgaggtcaggtatgaacttgaaagtggactccatgaagcattggtggaaagaaaaattaaaataatctta | |
| | attgaatttacacctgttactgacttcacattcttgccccaatcactaaagcttttgaaatctcacagagttctgaagtggaa | |
| | ggccgataaatctctttcttataactcaaggttctggaagaaccttctttacttaatgcctgcaaaaacagtcaagccaggt | |
| | agagacgaaccggaagtcttgcctgttctttccgagtcttgataa | |
| CAR 20A10N | atggccttaccagtgaccgccttgctcctgccgctggccttgctgctccacgccgccaggccgcaggtgcagctggtt | 130 |
| CD28-CD28-CD28 | gaatctggcggcggacttgtgaagcctggcggatctctgagactgagctgtgccgccagcggcttcacctttagcacat | |
| 1XX 6XIL2NFAT | acgccatgagctggatcagacaggcccctggcaaaggcctggaatgggggcgtctattggcagagccggcagcac | |
| mCMV | ctactacagcgactctgtgaagggcagattcaccatcagccgggacaacgccaagaacagcctgtacctgcagatga | |
| IL 18Ralpha | actccctgagagccgaggacaccgccgtgtactattgtgccagaggacccatctacaacgactacgacgagttcgcct | |
| | attggggccagggcacactggtcacagtttctagcggcggtggcggaagcggaggcggtggctccggtggcggag | |
| | gcagcgaaattgtgctgacacagagccccgccacactgtcactttctccaggcgaaagagccacactgagctgcaag | |
| | agcagccagagcgtgctgtactccagcaaccagaagaactacctggcctggtatcagcagaagcccggccaagctc | |
| | ctcggctgctgatctattgggccagcacaagagagagcggcatccctgccagattttctggcagcggctctggcaccg | |
| | atttcaccctgaccataagcagcctggaacctgaggacttcgccgtgtattactgccaccagtacctgagcagcctgac | |
| | ctttggcggaggcaccaaggtggaaatcaagcggaagcacctgtgtccttctccactgttccccggccctagcaagcc | |
| | tttctgggtcctggtggtggtgggcggagtgctggcctgctacagcctgctggtgaccgtggcctttatcatcttctgggt | |
| | gaggagtaagaggagcaggctcctgcacagtgactacatgaacatgactcctagaagacctgggcctaccagaaag | |
| | cattaccagccctatgccccaccacgcgacttcgcagcctatcgctccagagtgaagttcagcaggagcgcagacgc | |
| | ccccgcgtacaagcagggccagaaccagctctataacgagctcaatctaggacgaagagaggagtacgatgttttgg | |
| | acaagagacgtggccgggaccctgagatggggggaaagccgagaaggaagaaccctcaggaaggcctgttcaat | |
| | gaactgcagaaagataagatggcggaggccttcagtgagattgggatgaaaggcgagcgccggaggggcaaggg | |
| | gcacgatggccttttccagggtctcagtacagccaccaaggacaccttcgacgcccttcacatgcaggccctgccccc | |
| | tcgctgataagtttaaactgccagaacattactctggcctaactggccggtaccggaggaaaaactgtttcatacagaag | |
| | gcgtggaggaaaaactgtttcatacagaaggcgtggaggaaaaactgtttcatacagaaggcgtggaggaaaaactg | |
| | tttcatacagaaggcgtggaggaaaaactgtttcatacagaaggcgtggaggaaaaactgtttcatacagaaggcgtac | |
| | tagttaggcgtgtacggtgggaggcctatataagcagagctcgtttagtgaaccgtcagatcgcctggagacgccatcc | |
| | acgctgttttgacctccatagaagacaccgggaccgatccagcctcgagagacccaatgctagccaccatgaattgta | |
| | gagaattacccttgaccctttgggtgcttatatctgtaagcactgcagaatcttgtacttcacgtccccacattactgtggtt | |
| | gaaggggaacctttctatctgaaacattgctcgtgttcacttgcacatgagattgaaacaaccaccaaaagctggtacaa | |
| | aagcagtggatcacaggaacatgtggagctgaacccaaggagttccagcagaattgctttgcatgattgtgttttggagt | |
| | tttggccagttgagttgaatgacacaggatcttactttttccaaatgaaaaattatactcagaaatggaaattaaatgtcatc | |
| | agaagaaataaacacagctgtttcactgaaagacaagtaactagtaaaattgtggaagttaaaaaattttttcagataacct | |
| | gtgaaaacagttactatcaaacactggtcaacagcacatcattgtataagaactgtaaaaagctactactggagaacaat | |
| | aaaaacccaacgataaagaagaacgccgagtttgaagatcaggggtattactcctgcgtgcatttccttcatcataatgg | |
| | aaaactatttaatatcaccaaaaccttcaatataacaatagtggaagatcgcagtaatatagttccggttcttcttggaccaa | |
| | agcttaaccatgttgcagtggaattaggaaaaaacgtaaggctcaactgctctgctttgctgaatgaagaggatgtaattt | |
| | attggatgttcggggaagaaaatggatcggatcctaatatacatgaagagaaagaaatgagaattatgactccagaagg | |
| | caaatggcatgcttcaaaagtattgagaattgaaaatattggtgaaagcaatctaaatgttttatataattgcactgtggcca | |
| | gcacgggaggcacagacaccaaaagcttcatcttggtgagaaaagcagacatggctgatatcccaggccacgtcttc | |
| | acaagaggaatgatcatagctgttttgatcttggtggcagtagtgtgcctagtgactgtgtgtgtcatttatagagttgactt | |
| | ggttctattttatagacatttaacgagaagagatgaaacattaacagatggaaaaacatatgatgcttttgtgtcttacctaa | |
| | aagaatgccgacctgaaaatggagaggagcacacctttgctgtggagattttgcccagggtgttggagaaacattttgg | |
| | gtataagttatgcatatttgaaagggatgtagtgcctggaggagctgttgttgatgaaatccactcactgatagagaaaag | |
| | ccgaagactaatcattgtcctaagtaaaagttatatgtctaatgaggtcaggtatgaacttgaaagtggactccatgaagc | |
| | attggtggaaagaaaaattaaaataatcttaattgaatttacacctgttactgacttcacattcttgccccaatcactaaagct | |
| | tttgaaatctcacagagttctgaagtggaaggccgataaatctctttcttataactcaaggttctggaagaaccttctttactt | |
| | aatgcctgcaaaaacagtcaagccaggtagagacgaaccggaagtcttgcctgttctttccgagtcttgataa | |
| CAR 20A10N | atggccttaccagtgaccgccttgctcctgccgctggccttgctgctccacgccgccaggccgcaggtgcagctggtt | 131 |
| CD28-CD28-CD28 | gaatctggcggcggacttgtgaagcctggcggatctctgagactgagctgtgccgccagcggcttcacctttagcacat | |
| 1XX3XIL2NFAT | acgccatgagctggatcagacaggcccctggcaaaggcctggaatgggtggcgtctattggcagagccggcagcac | |
| mCMV | ctactacagcgactctgtgaagggcagattcaccatcagccgggacaacgccaagaacagcctgtacctgcagatga | |
| IL18Ralpha | actccctgagagccgaggacaccgccgtgtactattgtgccagaggacccatctacaacgactacgacgagttcgcct | |
| | attggggccagggcacactggtcacagtttctagcggcggtggcggaagcggaggcggtggctccggtggcggag | |
| | gcagcgaaattgtgctgacacagagccccgccacactgtcactttctccaggcgaaagagccacactgagctgcaag | |
| | agcagccagagcgtgctgtactccagcaaccagaagaactacctggcctggtatcagcagaagcccggccaagctc | |
| | ctcggctgctgatctattgggccagcacaagagagagcggcatccctgccagattttctggcagcggctctggcaccg | |
| | atttcaccctgaccataagcagcctggaacctgaggacttcgccgtgtattactgccaccagtacctgagcagcctgac | |
| | ctttggcggaggcaccaaggtggaaatcaagcggaagcacctgtgtccttctccactgttccccggccctagcaagcc | |
| | tttctgggtcctggtggtggtgggcggagtgctggcctgctacagcctgctggtgaccgtggcctttatcatcttctgggt | |
| | gaggagtaagaggagcaggctcctgcacagtgactacatgaacatgactcctagaagacctgggcctaccagaaag | |
| | cattaccagccctatgccccaccacgcgacttcgcagcctatcgctccagagtgaagttcagcaggagcgcagacgc | |
| | ccccgcgtacaagcagggccagaaccagctctataacgagctcaatctaggacgaagagaggagtacgatgttttgg | |
| | acaagagacgtggccgggaccctgagatggggggaaagccgagaaggaagaaccctcaggaaggcctgttcaat | |
| | gaactgcagaaagataagatggcggaggccttcagtgagattgggatgaaaggcgagcgccggaggggcaaggg | |
| | gcacgatggccttttccagggtctcagtacagccaccaaggacaccttcgacgcccttcacatgcaggccctgccccc | |
| | tcgctgataagtttaaactgccagaacattactctggcctaactggccggtaccggaggaaaaactgtttcatacagaag | |
| | gcgtggaggaaaaactgtttcatacagaaggcgtggaggaaaaactgtttcatacagaaggcgtactagttaggcgtgt | |
| | acggtgggaggcctatataagcagagctcgtttagtgaaccgtcagatcgcctggagacgccatccacgctgttttgac | |
| | ctccatagaagacaccgggaccgatccagcctcgagagacccaatgctagccaccatgaattgtagagaattaccctt | |
| | gaccctttgggtgcttatatctgtaagcactgcagaatcttgtacttcacgtccccacattactgtggttgaaggggaacct | |
| | ttctatctgaaacattgctcgtgttcacttgcacatgagattgaaacaaccaccaaaagctggtacaaaagcagtggatca | |
| | caggaacatgtggagctgaacccaaggagttccagcagaattgctttgcatgattgtgttttggagttttggccagttgag | |
| | ttgaatgacacaggatcttactttttccaaatgaaaaattatactcagaaatggaaattaaatgtcatcagaagaaataaac | |
| | acagctgtttcactgaaagacaagtaactagtaaaattgtggaagttaaaaaattttttcagataacctgtgaaaacagtta | |
| | ctatcaaacactggtcaacagcacatcattgtataagaactgtaaaaagctactactggagaacaataaaaacccaacg | |
| | ataaagaagaacgccgagtttgaagatcaggggtattactcctgcgtgcatttccttcatcataatggaaaactatttaata | |
| | tcaccaaaaccttcaatataacaatagtggaagatcgcagtaatatagttccggttcttcttggaccaaagcttaaccatgt | |
| | tgcagtggaattaggaaaaaacgtaaggctcaactgctctgctttgctgaatgaagaggatgtaatttattggatgttcgg | |
| | ggaagaaaatggatcggatcctaatatacatgaagagaaagaaatgagaattatgactccagaaggcaaatggcatgc | |
| | ttcaaaagtattgagaattgaaaatattggtgaaagcaatctaaatgttttatataattgcactgtggccagcacgggaggc | |
| | acagacaccaaaagcttcatcttggtgagaaaagcagacatggctgatatcccaggccacgtcttcacaagaggaatg | |
| | atcatagctgttttgatcttggtggcagtagtgtgcctagtgactgtgtgtgtcatttatagagttgacttggttctattttatag | |
| | acatttaacgagaagagatgaaacattaacagatggaaaaacatatgatgcttttgtgtcttacctaaaagaatgccgacc | |
| | tgaaaatggagaggagcacacctttgctgtggagattttgcccagggtgttggagaaacattttgggtataagttatgcat | |
| | atttgaaagggatgtagtgcctggaggagctgttgttgatgaaatccactcactgatagagaaaagccgaagactaatc | |
| | attgtcctaagtaaaagttatatgtctaatgaggtcaggtatgaacttgaaagtggactccatgaagcattggtggaaaga | |
| | aaaattaaaataatcttaattgaatttacacctgttactgacttcacattcttgccccaatcactaaagcttttgaaatctcaca | |
| | gagttctgaagtggaaggccgataaatctctttcttataactcaaggttctggaagaaccttctttacttaatgcctgcaaaa | |
| | acagtcaagccaggtagagacgaaccggaagtcttgcctgttctttccgagtcttgataa | |
| CAR C2 CD8- | atggccttaccagtgaccgccttgctcctgccgctggccttgctgctccacgccgccaggccggaggtgcagctggtg | 132 |
| CD8-41BBCD3 | gagtctgggggaggcctggtcaagcctggggggtccctgagactctcctgtgcagcctctggattcaccttcagtggc | |
| 6XFOXP3NFAT | tatgccatgagctgggtccgccaggctccagggaaggggctggagtgggtctcaaccattagtagtggggaaccta | |
| mCMV | catatactaccccgactcagtgaagggccgattcaccatctccagagacaacgccaagaactcactgtatctgcaaatg | |
| IL18Ralpha-T2A- | aacagcctgagagccgaggacacggccgtgtattactgtgcgagacttgggggggataattactacgaatacttcgat | |
| IL18Rb | gtctggggcaaagggaccacggtcaccgtctcctccggcggtggcggatccggcggtggcggatccggcggtggc | |
| | ggatccgacattgtgctgacccagtctccagcctccttggccgtgtctccaggacagagggccaccatcacctgcaga | |
| | gccagtaagagtgtcagtaccagcggatactcctacatgcactggtatcagcagaaaccaggacaacctcctaaactc | |
| | ctgatttacctggcatccaatctggagagcggggtcccagccaggttcagcggcagtgggtctgggaccgatttcacc | |
| | ctcacaattaatcctgtggaagctaatgatactgcaaattattactgtcagcacagtagggagctgcctttcacattcggc | |
| | ggagggaccaaggtggagatcaaacgaactacaacaacccctgcccccagacctcctaccccagcccctacaattg | |
| | ccagccagcctctgagcctgaggcccgaggcttgtagacctgctgctggcggagccgtgcacaccagaggactgga | |
| | tttcgcctgcgacatctacatctgggcgcccttggccgggacttgtggggtccttctcctgtcactggttatcaccctttact | |
| | gcaaacggggcagaaagaaactcctgtatatattcaaacaaccatttatgagaccagtacaaactactcaagaggaag | |
| | atggctgtagctgccgatttccagaagaagaagaaggaggatgtgaactgagagtgaagttcagcaggagcgcaga | |
| | cgcccccgcgtacaagcagggccagaaccagctctataacgagctcaatctaggacgaagagaggagtacgatgttt | |
| | tggacaagagacgtggccgggaccctgagatggggggaaagccgagaaggaagaaccctcaggaaggcctgtac | |
| | aatgaactgcagaaagataagatggcggaggcctacagtgagattgggatgaaaggcgagcgccggaggggcaag | |
| | gggcacgatggcctttaccagggtctcagtacagccaccaaggacacctacgacgcccttcacatgcaggccctgcc | |
| | ccctcgctgataagtttaaactgccagaacatttctctggcctaactggccggtaccggcttcattttttccatttactgcag | |
| | aggcttcattttttccatttactgcagaggcttcattttttccatttactgcagaggcttcattttttccatttactgcagaggctt | |
| | cattttttccatttactgcagaggcttcattttttccatttactgcagaactagttaggcgtgtacggtgggaggcctatataa | |
| | gcagagctcgtttagtgaaccgtcagatcgcctggagacgccatccacgctgttttgacctccatagaagacaccggg | |
| | accgatccagcctcgagagacccaatgctagccaccatgaattgtagagaattacccttgaccctttgggtgcttatatc | |
| | tgtaagcactgcagaatcttgtacttcacgtccccacattactgtggttgaaggggaacctttctatctgaaacattgctcgt | |
| | gttcacttgcacatgagattgaaacaaccaccaaaagctggtacaaaagcagtggatcacaggaacatgtggagctga | |
| | acccaaggagttccagcagaattgctttgcatgattgtgttttggagttttggccagttgagttgaatgacacaggatctta | |
| | ctttttccaaatgaaaaattatactcagaaatggaaattaaatgtcatcagaagaaataaacacagctgtttcactgaaaga | |
| | caagtaactagtaaaattgtggaagttaaaaaattttttcagataacctgtgaaaacagttactatcaaacactggtcaaca | |
| | gcacatcattgtataagaactgtaaaaagctactactggagaacaataaaaacccaacgataaagaagaacgccgagt | |
| | ttgaagatcaggggtattactcctgcgtgcatttccttcatcataatggaaaactatttaatatcaccaaaaccttcaatataa | |
| | caatagtggaagatcgcagtaatatagttccggttcttcttggaccaaagcttaaccatgttgcagtggaattaggaaaaa | |
| | acgtaaggctcaactgctctgctttgctgaatgaagaggatgtaatttattggatgttcggggaagaaaatggatcggat | |
| | cctaatatacatgaagagaaagaaatgagaattatgactccagaaggcaaatggcatgcttcaaaagtattgagaattga | |
| | aaatattggtgaaagcaatctaaatgttttatataattgcactgtggccagcacgggaggcacagacaccaaaagcttca | |
| | tcttggtgagaaaagcagacatggctgatatcccaggccacgtcttcacaagaggaatgatcatagctgttttgatcttgg | |
| | tggcagtagtgtgcctagtgactgtgtgtgtcatttatagagttgacttggttctattttatagacatttaacgagaagagatg | |
| | aaacattaacagatggaaaaacatatgatgcttttgtgtcttacctaaaagaatgccgacctgaaaatggagaggagcac | |
| | acctttgctgtggagattttgcccagggtgttggagaaacattttgggtataagttatgcatatttgaaagggatgtagtgc | |
| | ctggaggagctgttgttgatgaaatccactcactgatagagaaaagccgaagactaatcattgtcctaagtaaaagttata | |
| | tgtctaatgaggtcaggtatgaacttgaaagtggactccatgaagcattggtggaaagaaaaattaaaataatcttaattg | |
| | aatttacacctgttactgacttcacattcttgccccaatcactaaagcttttgaaatctcacagagttctgaagtggaaggcc | |
| | gataaatctctttcttataactcaaggttctggaagaaccttctttacttaatgcctgcaaaaacagtcaagccaggtagag | |
| | acgaaccggaagtcttgcctgttctttccgagtctggaagcggtgagggaagaggtagcctgctcacgtgcggtgatg | |
| | tggaggagaaccctgggcctatgctctgtttgggctggatatttctttggcttgttgcaggagagcgaattaaaggattta | |
| | atatttcaggttgttccacaaaaaaactcctttggacatattctacaaggagtgaagaggaatttgtcttattttgtgatttacc | |
| | agagccacagaaatcacatttctgccacagaaatcgactctcaccaaaacaagtccctgagcacctgcccttcatgggt | |
| | agtaacgacctatctgatgtccaatggtaccaacaaccttcgaatggagatccattagaggacattaggaaaagctatcc | |
| | tcacatcattcaggacaaatgtacccttcactttttgaccccaggggtgaataattctgggtcatatatttgtagacccaag | |
| | atgattaagagcccctatgatgtagcctgttgtgtcaagatgattttagaagttaagccccagacaaatgcatcctgtgagt | |
| | attccgcatcacataagcaagacctacttcttgggagcactggctctatttcttgccccagtctcagctgccaaagtgatg | |
| | cacaaagtccagcggtaacctggtacaagaatggaaaactcctctctgtggaaaggagcaaccgaatcgtagtggatg | |
| | aagtttatgactatcaccagggcacatatgtatgtgattacactcagtcggatactgtgagttcgtggacagtcagagctg | |
| | ttgttcaagtgagaaccattgtgggagacactaaactcaaaccagatattctggatcctgtcgaggacacactggaagta | |
| | gaacttggaaagcctttaactattagctgcaaagcacgatttggctttgaaagggtctttaaccctgtcataaaatggtaca | |
| | tcaaagattctgacctagagtgggaagtctcagtacctgaggcgaaaagtattaaatccactttaaaggatgaaatcattg | |
| | agcgtaatatcatcttggaaaaagtcactcagcgtgatcttcgcaggaagtttgtttgctttgtccagaactccattggaaa | |
| | cacaacccagtccgtccaactgaaagaaaagagaggagtggtgctcctgtacatcctgcttggcaccatcgggaccct | |
| | ggtggccgtgctggcggcgagtgccctcctctacaggcactggattgaaatagtgctgctgtaccggacctaccagag | |
| | caaggatcagacgcttggggataaaaaggattttgatgctttcgtatcctatgcaaaatggagctcttttccaagtgaggc | |
| | cacttcatctctgagtgaagaacacttggccctgagcctatttcctgatgttttagaaaacaaatatggatatagcctgtgtt | |
| | tgcttgaaagagatgtggctccaggaggagtgtatgcagaagacattgtgagcattattaagagaagcagaagaggaa | |
| | tatttatcttgagccccaactatgtcaatggacccagtatctttgaactacaagcagcagtgaatcttgccttggatgatca | |
| | aacactgaaactcattttaattaagttctgttacttccaagagccagagtctctacctcatctcgtgaaaaaagctctcagg | |
| | gttttgcccacagttacttggagaggcttaaaatcagttcctcccaattctaggttctgggccaaaatgcgctaccacatg | |
| | cctgtgaaaaactctcagggattcacgtggaaccagctcagaattacctctaggatttttcagtggaaaggactcagtag | |
| | aacagaaaccactgggaggagctcccagcctaaggaatggtgataa | |
| CAR C2 CD8- | atggccttaccagtgaccgccttgctcctgccgctggccttgctgctccacgccgccaggccggaggtgcagctggtg | 133 |
| CD8-41BBCD3 | gagtctgggggaggcctggtcaagcctggggggtccctgagactctcctgtgcagcctctggattcaccttcagtggc | |
| 3XFOXP3NFAT | tatgccatgagctgggtccgccaggctccagggaaggggctggagtgggtctcaaccattagtagtggcggaaccta | |
| mCMV | catatactaccccgactcagtgaagggccgattcaccatctccagagacaacgccaagaactcactgtatctgcaaatg | |
| IL18Ralpha-T2A- | aacagcctgagagccgaggacacggccgtgtattactgtgcgagacttgggggggataattactacgaatacttcgat | |
| IL18Rb | gtctggggcaaagggaccacggtcaccgtctcctccggcggtggcggatccggcggtggcggatccggcggtggc | |
| | ggatccgacattgtgctgacccagtctccagcctccttggccgtgtctccaggacagagggccaccatcacctgcaga | |
| | gccagtaagagtgtcagtaccagcggatactcctacatgcactggtatcagcagaaaccaggacaacctcctaaactc | |
| | ctgatttacctggcatccaatctggagagcggggtcccagccaggttcagcggcagtgggtctgggaccgatttcacc | |
| | ctcacaattaatcctgtggaagctaatgatactgcaaattattactgtcagcacagtagggagctgcctttcacattcggc | |
| | ggagggaccaaggtggagatcaaacgaactacaacaacccctgcccccagacctcctaccccagcccctacaattg | |
| | ccagccagcctctgagcctgaggcccgaggcttgtagacctgctgctggcggagccgtgcacaccagaggactgga | |
| | tttcgcctgcgacatctacatctgggcgcccttggccgggacttgtggggtccttctcctgtcactggttatcaccctttact | |
| | gcaaacggggcagaaagaaactcctgtatatattcaaacaaccatttatgagaccagtacaaactactcaagaggaag | |
| | atggctgtagctgccgatttccagaagaagaagaaggaggatgtgaactgagagtgaagttcagcaggagcgcaga | |
| | cgcccccgcgtacaagcagggccagaaccagctctataacgagctcaatctaggacgaagagaggagtacgatgttt | |
| | tggacaagagacgtggccgggaccctgagatggggggaaagccgagaaggaagaaccctcaggaaggcctgtac | |
| | aatgaactgcagaaagataagatggcggaggcctacagtgagattgggatgaaaggcgagcgccggaggggcaag | |
| | gggcacgatggcctttaccagggtctcagtacagccaccaaggacacctacgacgcccttcacatgcaggccctgcc | |
| | ccctcgctgataagtttaaactgccagaacatttctctggcctaactggccggtaccggcttcattttttccatttactgcag | |
| | aggcttcattttttccatttactgcagaggcttcattttttccatttactgcagaactagttaggcgtgtacggtgggaggcct | |
| | atataagcagagctcgtttagtgaaccgtcagatcgcctggagacgccatccacgctgttttgacctccatagaagaca | |
| | ccgggaccgatccagcctcgagagacccaatgctagccaccatgaattgtagagaattacccttgaccctttgggtgc | |
| | ttatatctgtaagcactgcagaatcttgtacttcacgtccccacattactgtggttgaaggggaacctttctatctgaaacatt | |
| | gctcgtgttcacttgcacatgagattgaaacaaccaccaaaagctggtacaaaagcagtggatcacaggaacatgtgg | |
| | agctgaacccaaggagttccagcagaattgctttgcatgattgtgttttggagttttggccagttgagttgaatgacacag | |
| | gatcttactttttccaaatgaaaaattatactcagaaatggaaattaaatgtcatcagaagaaataaacacagctgtttcact | |
| | gaaagacaagtaactagtaaaattgtggaagttaaaaaattttttcagataacctgtgaaaacagttactatcaaacactg | |
| | gtcaacagcacatcattgtataagaactgtaaaaagctactactggagaacaataaaaacccaacgataaagaagaac | |
| | gccgagtttgaagatcaggggtattactcctgcgtgcatttccttcatcataatggaaaactatttaatatcaccaaaacctt | |
| | caatataacaatagtggaagatcgcagtaatatagttccggttcttcttggaccaaagcttaaccatgttgcagtggaatta | |
| | ggaaaaaacgtaaggctcaactgctctgctttgctgaatgaagaggatgtaatttattggatgttcggggaagaaaatgg | |
| | atcggatcctaatatacatgaagagaaagaaatgagaattatgactccagaaggcaaatggcatgcttcaaaagtattga | |
| | gaattgaaaatattggtgaaagcaatctaaatgttttatataattgcactgtggccagcacgggaggcacagacaccaaa | |
| | agcttcatcttggtgagaaaagcagacatggctgatatcccaggccacgtcttcacaagaggaatgatcatagctgtttt | |
| | gatcttggtggcagtagtgtgcctagtgactgtgtgtgtcatttatagagttgacttggttctattttatagacatttaacgag | |
| | aagagatgaaacattaacagatggaaaaacatatgatgcttttgtgtcttacctaaaagaatgccgacctgaaaatggag | |
| | aggagcacacctttgctgtggagattttgcccagggtgttggagaaacattttgggtataagttatgcatatttgaaaggg | |
| | atgtagtgcctggaggagctgttgttgatgaaatccactcactgatagagaaaagccgaagactaatcattgtcctaagt | |
| | aaaagttatatgtctaatgaggtcaggtatgaacttgaaagtggactccatgaagcattggtggaaagaaaaattaaaat | |
| | aatcttaattgaatttacacctgttactgacttcacattcttgccccaatcactaaagcttttgaaatctcacagagttctgaag | |
| | tggaaggccgataaatctctttcttataactcaaggttctggaagaaccttctttacttaatgcctgcaaaaacagtcaagc | |
| | caggtagagacgaaccggaagtcttgcctgttctttccgagtctggaagcggtgagggaagaggtagcctgctcacgt | |
| | gcggtgatgtggaggagaaccctgggcctatgctctgtttgggctggatatttctttggcttgttgcaggagagcgaatta | |
| | aaggatttaatatttcaggttgttccacaaaaaaactcctttggacatattctacaaggagtgaagaggaatttgtcttatttt | |
| | gtgatttaccagagccacagaaatcacatttctgccacagaaatcgactctcaccaaaacaagtccctgagcacctgcc | |
| | cttcatgggtagtaacgacctatctgatgtccaatggtaccaacaaccttcgaatggagatccattagaggacattagga | |
| | aaagctatcctcacatcattcaggacaaatgtacccttcactttttgaccccaggggtgaataattctgggtcatatatttgt | |
| | agacccaagatgattaagagcccctatgatgtagcctgttgtgtcaagatgattttagaagttaagccccagacaaatgc | |
| | atcctgtgagtattccgcatcacataagcaagacctacttcttgggagcactggctctatttcttgccccagtctcagctgc | |
| | caaagtgatgcacaaagtccagcggtaacctggtacaagaatggaaaactcctctctgtggaaaggagcaaccgaat | |
| | cgtagtggatgaagtttatgactatcaccagggcacatatgtatgtgattacactcagtcggatactgtgagttcgtggac | |
| | agtcagagctgttgttcaagtgagaaccattgtgggagacactaaactcaaaccagatattctggatcctgtcgaggaca | |
| | cactggaagtagaacttggaaagcctttaactattagctgcaaagcacgatttggctttgaaagggtctttaaccctgtcat | |
| | aaaatggtacatcaaagattctgacctagagtgggaagtctcagtacctgaggcgaaaagtattaaatccactttaaagg | |
| | atgaaatcattgagcgtaatatcatcttggaaaaagtcactcagcgtgatcttcgcaggaagtttgtttgctttgtccagaa | |
| | ctccattggaaacacaacccagtccgtccaactgaaagaaaagagaggagtggtgctcctgtacatcctgcttggcac | |
| | catcgggaccctggtggccgtgctggcggcgagtgccctcctctacaggcactggattgaaatagtgctgctgtaccg | |
| | gacctaccagagcaaggatcagacgcttggggataaaaaggattttgatgctttcgtatcctatgcaaaatggagctcttt | |
| | tccaagtgaggccacttcatctctgagtgaagaacacttggccctgagcctatttcctgatgttttagaaaacaaatatgg | |
| | atatagcctgtgtttgcttgaaagagatgtggctccaggaggagtgtatgcagaagacattgtgagcattattaagagaa | |
| | gcagaagaggaatatttatcttgagccccaactatgtcaatggacccagtatctttgaactacaagcagcagtgaatcttg | |
| | ccttggatgatcaaacactgaaactcattttaattaagttctgttacttccaagagccagagtctctacctcatctcgtgaaa | |
| | aaagctctcagggttttgcccacagttacttggagaggcttaaaatcagttcctcccaattctaggttctgggccaaaatg | |
| | cgctaccacatgcctgtgaaaaactctcagggattcacgtggaaccagctcagaattacctctaggatttttcagtggaa | |
| | aggactcagtagaacagaaaccactgggaggagctcccagcctaaggaatggtgataa | |
| CAR C2 CD8- | atggccttaccagtgaccgccttgctcctgccgctggccttgctgctccacgccgccaggccggaggtgcagctggtg | 134 |
| CD8-41BBCD3 | gagtctgggggaggcctggtcaagcctggggggtccctgagactctcctgtgcagcctctggattcaccttcagtggc | |
| 6XIL2NFAT | tatgccatgagctgggtccgccaggctccagggaaggggctggagtgggtctcaaccattagtagtggcggaaccta | |
| mCMV | catatactaccccgactcagtgaagggccgattcaccatctccagagacaacgccaagaactcactgtatctgcaaatg | |
| IL18Ralpha-T2A- | aacagcctgagagccgaggacacggccgtgtattactgtgcgagacttgggggggataattactacgaatacttcgat | |
| IL18Rb | gtctggggcaaagggaccacggtcaccgtctcctccggcggtggcggatccggcggtggcggatccggcggtggc | |
| | ggatccgacattgtgctgacccagtctccagcctccttggccgtgtctccaggacagagggccaccatcacctgcaga | |
| | gccagtaagagtgtcagtaccagcggatactcctacatgcactggtatcagcagaaaccaggacaacctcctaaactc | |
| | ctgatttacctggcatccaatctggagagcggggtcccagccaggttcagcggcagtgggtctgggaccgatttcacc | |
| | ctcacaattaatcctgtggaagctaatgatactgcaaattattactgtcagcacagtagggagctgcctttcacattcggc | |
| | ggagggaccaaggtggagatcaaacgaactacaacaacccctgcccccagacctcctaccccagcccctacaattg | |
| | ccagccagcctctgagcctgaggcccgaggcttgtagacctgctgctggcggagccgtgcacaccagaggactgga | |
| | tttcgcctgcgacatctacatctgggcgcccttggccgggacttgtggggtccttctcctgtcactggttatcaccctttact | |
| | gcaaacggggcagaaagaaactcctgtatatattcaaacaaccatttatgagaccagtacaaactactcaagaggaag | |
| | atggctgtagctgccgatttccagaagaagaagaaggaggatgtgaactgagagtgaagttcagcaggagcgcaga | |
| | cgcccccgcgtacaagcagggccagaaccagctctataacgagctcaatctaggacgaagagaggagtacgatgttt | |
| | tggacaagagacgtggccgggaccctgagatggggggaaagccgagaaggaagaaccctcaggaaggcctgtac | |
| | aatgaactgcagaaagataagatggcggaggcctacagtgagattgggatgaaaggcgagcgccggaggggcaag | |
| | gggcacgatggcctttaccagggtctcagtacagccaccaaggacacctacgacgcccttcacatgcaggccctgcc | |
| | ccctcgctgataagtttaaactgccagaacatttctctggcctaactggccggtaccggaggaaaaactgtttcatacag | |
| | aaggcgtggaggaaaaactgtttcatacagaaggcgtggaggaaaaactgtttcatacagaaggcgtggaggaaaaa | |
| | ctgtttcatacagaaggcgtggaggaaaaactgtttcatacagaaggcgtggaggaaaaactgtttcatacagaaggc | |
| | gtactagttaggcgtgtacggtgggaggcctatataagcagagctcgtttagtgaaccgtcagatcgcctggagacgc | |
| | catccacgctgttttgacctccatagaagacaccgggaccgatccagcctcgagagacccaatgctagccaccatga | |
| | attgtagagaattacccttgaccctttgggtgcttatatctgtaagcactgcagaatcttgtacttcacgtccccacattactg | |
| | tggttgaaggggaacctttctatctgaaacattgctcgtgttcacttgcacatgagattgaaacaaccaccaaaagctggt | |
| | acaaaagcagtggatcacaggaacatgtggagctgaacccaaggagttccagcagaattgctttgcatgattgtgttttg | |
| | gagttttggccagttgagttgaatgacacaggatcttactttttccaaatgaaaaattatactcagaaatggaaattaaatgt | |
| | catcagaagaaataaacacagctgtttcactgaaagacaagtaactagtaaaattgtggaagttaaaaaattttttcagata | |
| | acctgtgaaaacagttactatcaaacactggtcaacagcacatcattgtataagaactgtaaaaagctactactggagaa | |
| | caataaaaacccaacgataaagaagaacgccgagtttgaagatcaggggtattactcctgcgtgcatttccttcatcata | |
| | atggaaaactatttaatatcaccaaaaccttcaatataacaatagtggaagatcgcagtaatatagttccggttcttcttgga | |
| | ccaaagcttaaccatgttgcagtggaattaggaaaaaacgtaaggctcaactgctctgctttgctgaatgaagaggatgt | |
| | aatttattggatgttcggggaagaaaatggatcggatcctaatatacatgaagagaaagaaatgagaattatgactccag | |
| | aaggcaaatggcatgcttcaaaagtattgagaattgaaaatattggtgaaagcaatctaaatgttttatataattgcactgtg | |
| | gccagcacgggaggcacagacaccaaaagcttcatcttggtgagaaaagcagacatggctgatatcccaggccacg | |
| | tcttcacaagaggaatgatcatagctgttttgatcttggtggcagtagtgtgcctagtgactgtgtgtgtcatttatagagtt | |
| | gacttggttctattttatagacatttaacgagaagagatgaaacattaacagatggaaaaacatatgatgcttttgtgtctta | |
| | cctaaaagaatgccgacctgaaaatggagaggagcacacctttgctgtggagattttgcccagggtgttggagaaaca | |
| | ttttgggtataagttatgcatatttgaaagggatgtagtgcctggaggagctgttgttgatgaaatccactcactgatagag | |
| | aaaagccgaagactaatcattgtcctaagtaaaagttatatgtctaatgaggtcaggtatgaacttgaaagtggactccat | |
| | gaagcattggtggaaagaaaaattaaaataatcttaattgaatttacacctgttactgacttcacattcttgccccaatcact | |
| | aaagcttttgaaatctcacagagttctgaagtggaaggccgataaatctctttcttataactcaaggttctggaagaacctt | |
| | ctttacttaatgcctgcaaaaacagtcaagccaggtagagacgaaccggaagtcttgcctgttctttccgagtctggaag | |
| | cggtgagggaagaggtagcctgctcacgtgcggtgatgtggaggagaaccctgggcctatgctctgtttgggctggat | |
| | atttctttggcttgttgcaggagagcgaattaaaggatttaatatttcaggttgttccacaaaaaaactcctttggacatattct | |
| | acaaggagtgaagaggaatttgtcttattttgtgatttaccagagccacagaaatcacatttctgccacagaaatcgactct | |
| | caccaaaacaagtccctgagcacctgcccttcatgggtagtaacgacctatctgatgtccaatggtaccaacaaccttcg | |
| | aatggagatccattagaggacattaggaaaagctatcctcacatcattcaggacaaatgtacccttcactttttgacccca | |
| | ggggtgaataattctgggtcatatatttgtagacccaagatgattaagagcccctatgatgtagcctgttgtgtcaagatg | |
| | attttagaagttaagccccagacaaatgcatcctgtgagtattccgcatcacataagcaagacctacttcttgggagcact | |
| | ggctctatttcttgccccagtctcagctgccaaagtgatgcacaaagtccagcggtaacctggtacaagaatggaaaac | |
| | tcctctctgtggaaaggagcaaccgaatcgtagtggatgaagtttatgactatcaccagggcacatatgtatgtgattaca | |
| | ctcagtcggatactgtgagttcgtggacagtcagagctgttgttcaagtgagaaccattgtgggagacactaaactcaaa | |
| | ccagatattctggatcctgtcgaggacacactggaagtagaacttggaaagcctttaactattagctgcaaagcacgattt | |
| | ggctttgaaagggtctttaaccctgtcataaaatggtacatcaaagattctgacctagagtgggaagtctcagtacctgag | |
| | gcgaaaagtattaaatccactttaaaggatgaaatcattgagcgtaatatcatcttggaaaaagtcactcagcgtgatcttc | |
| | gcaggaagtttgtttgctttgtccagaactccattggaaacacaacccagtccgtccaactgaaagaaaagagaggagt | |
| | ggtgctcctgtacatcctgcttggcaccatcgggaccctggtggccgtgctggcggcgagtgccctcctctacaggca | |
| | ctggattgaaatagtgctgctgtaccggacctaccagagcaaggatcagacgcttggggataaaaaggattttgatgctt | |
| | tcgtatcctatgcaaaatggagctcttttccaagtgaggccacttcatctctgagtgaagaacacttggccctgagcctatt | |
| | tcctgatgttttagaaaacaaatatggatatagcctgtgtttgcttgaaagagatgtggctccaggaggagtgtatgcaga | |
| | agacattgtgagcattattaagagaagcagaagaggaatatttatcttgagccccaactatgtcaatggacccagtatctt | |
| | tgaactacaagcagcagtgaatcttgccttggatgatcaaacactgaaactcattttaattaagttctgttacttccaagagc | |
| | cagagtctctacctcatctc gtgaaaaaagctctcagggttttgcccacagttacttggagaggcttaaaatcagttcctcc | |
| | caattctaggttctgggccaaaatgcgctaccacatgcctgtgaaaaactctcagggattcacgtggaaccagctcaga | |
| | attacctctaggatttttcagtggaaaggactcagtagaacagaaaccactgggaggagctcccagcctaaggaatggt | |
| | gataa | |
| CAR C2 CD8- | atggccttaccagtgaccgccttgctcctgccgctggccttgctgctccacgccgccaggccggaggtgcagctggtg | 135 |
| CD8-41BBCD3 | gagtctgggggaggcctggtcaagcctggggggtccctgagactctcctgtgcagcctctggattcaccttcagtggc | |
| 3XIL2NFAT | tatgccatgagctgggtccgccaggctccagggaaggggctggagtgggtctcaaccattagtagtggcggaaccta | |
| mCMV | catatactaccccgactcagtgaagggccgattcaccatctccagagacaacgccaagaactcactgtatctgcaaatg | |
| IL18Ralpha-T2A- | aacagcctgagagccgaggacacggccgtgtattactgtgcgagacttgggggggataattactacgaatacttcgat | |
| IL18Rb | gtctggggcaaagggaccacggtcaccgtctcctccggcggtggcggatccggcggtggcggatccggcggtggc | |
| | ggatccgacattgtgctgacccagtctccagcctccttggccgtgtctccaggacagagggccaccatcacctgcaga | |
| | gccagtaagagtgtcagtaccagcggatactcctacatgcactggtatcagcagaaaccaggacaacctcctaaactc | |
| | ctgatttacctggcatccaatctggagagcggggtcccagccaggttcagcggcagtgggtctgggaccgatttcacc | |
| | ctcacaattaatcctgtggaagctaatgatactgcaaattattactgtcagcacagtagggagctgcctttcacattcggc | |
| | ggagggaccaaggtggagatcaaacgaactacaacaacccctgcccccagacctcctaccccagcccctacaattg | |
| | ccagccagcctctgagcctgaggcccgaggcttgtagacctgctgctggcggagccgtgcacaccagaggactgga | |
| | tttcgcctgcgacatctacatctgggcgcccttggccgggacttgtggggtccttctcctgtcactggttatcaccctttact | |
| | gcaaacggggcagaaagaaactcctgtatatattcaaacaaccatttatgagaccagtacaaactactcaagaggaag | |
| | atggctgtagctgccgatttccagaagaagaagaaggaggatgtgaactgagagtgaagttcagcaggagcgcaga | |
| | cgcccccgcgtacaagcagggccagaaccagctctataacgagctcaatctaggacgaagagaggagtacgatgttt | |
| | tggacaagagacgtggccgggaccctgagatggggggaaagccgagaaggaagaaccctcaggaaggcctgtac | |
| | aatgaactgcagaaagataagatggcggaggcctacagtgagattgggatgaaaggcgagcgccggaggggcaag | |
| | gggcacgatggcctttaccagggtctcagtacagccaccaaggacacctacgacgcccttcacatgcaggccctgcc | |
| | ccctcgctgataagtttaaactgccagaacatttctctggcctaactggccggtaccggaggaaaaactgtttcatacag | |
| | aaggcgtggaggaaaaactgtttcatacagaaggcgtggaggaaaaactgtttcatacagaaggcgtactagttaggc | |
| | gtgtacggtgggaggcctatataagcagagctcgtttagtgaaccgtcagatcgcctggagacgccatccacgctgttt | |
| | tgacctccatagaagacaccgggaccgatccagcctcgagagacccaatgctagccaccatgaattgtagagaatta | |
| | cccttgaccctttgggtgcttatatctgtaagcactgcagaatcttgtacttcacgtccccacattactgtggttgaagggg | |
| | aacctttctatctgaaacattgctcgtgttcacttgcacatgagattgaaacaaccaccaaaagctggtacaaaagcagtg | |
| | gatcacaggaacatgtggagctgaacccaaggagttccagcagaattgctttgcatgattgtgttttggagttttggcca | |
| | gttgagttgaatgacacaggatcttactttttccaaatgaaaaattatactcagaaatggaaattaaatgtcatcagaagaa | |
| | ataaacacagctgtttcactgaaagacaagtaactagtaaaattgtggaagttaaaaaattttttcagataacctgtgaaaa | |
| | cagttactatcaaacactggtcaacagcacatcattgtataagaactgtaaaaagctactactggagaacaataaaaacc | |
| | caacgataaagaagaacgccgagtttgaagatcaggggtattactcctgcgtgcatttccttcatcataatggaaaactat | |
| | ttaatatcaccaaaaccttcaatataacaatagtggaagatcgcagtaatatagttccggttcttcttggaccaaagcttaa | |
| | ccatgttgcagtggaattaggaaaaaacgtaaggctcaactgctctgctttgctgaatgaagaggatgtaatttattggat | |
| | gttcggggaagaaaatggatcggatcctaatatacatgaagagaaagaaatgagaattatgactccagaaggcaaatg | |
| | gcatgcttcaaaagtattgagaattgaaaatattggtgaaagcaatctaaatgttttatataattgcactgtggccagcacg | |
| | ggaggcacagacaccaaaagcttcatcttggtgagaaaagcagacatggctgatatcccaggccacgtcttcacaag | |
| | aggaatgatcatagctgttttgatcttggtggcagtagtgtgcctagtgactgtgtgtgtcatttatagagttgacttggttct | |
| | attttatagacatttaacgagaagagatgaaacattaacagatggaaaaacatatgatgcttttgtgtcttacctaaaagaat | |
| | gccgacctgaaaatggagaggagcacacctttgctgtggagattttgcccagggtgttggagaaacattttgggtataa | |
| | gttatgcatatttgaaagggatgtagtgcctggaggagctgttgttgatgaaatccactcactgatagagaaaagccgaa | |
| | gactaatcattgtcctaagtaaaagttatatgtctaatgaggtcaggtatgaacttgaaagtggactccatgaagcattggt | |
| | ggaaagaaaaattaaaataatcttaattgaatttacacctgttactgacttcacattcttgccccaatcactaaagcttttgaa | |
| | atctcacagagttctgaagtggaaggccgataaatctctttcttataactcaaggttctggaagaaccttctttacttaatgc | |
| | ctgcaaaaacagtcaagccaggtagagacgaaccggaagtcttgcctgttctttccgagtctggaagcggtgagggaa | |
| | gaggtagcctgctcacgtgcggtgatgtggaggagaaccctgggcctatgctctgtttgggctggatatttctttggcttg | |
| | ttgcaggagagcgaattaaaggatttaatatttcaggttgttccacaaaaaaactcctttggacatattctacaaggagtga | |
| | agaggaatttgtcttattttgtgatttaccagagccacagaaatcacatttctgccacagaaatcgactctcaccaaaacaa | |
| | gtccctgagcacctgcccttcatgggtagtaacgacctatctgatgtccaatggtaccaacaaccttcgaatggagatcc | |
| | attagaggacattaggaaaagctatcctcacatcattcaggacaaatgtacccttcactttttgaccccaggggtgaataa | |
| | ttctgggtcatatatttgtagacccaagatgattaagagcccctatgatgtagcctgttgtgtcaagatgattttagaagtta | |
| | agccccagacaaatgcatcctgtgagtattccgcatcacataagcaagacctacttcttgggagcactggctctatttctt | |
| | gccccagtctcagctgccaaagtgatgcacaaagtccagcggtaacctggtacaagaatggaaaactcctctctgtgg | |
| | aaaggagcaaccgaatcgtagtggatgaagtttatgactatcaccagggcacatatgtatgtgattacactcagtcggat | |
| | actgtgagttcgtggacagtcagagctgttgttcaagtgagaaccattgtgggagacactaaactcaaaccagatattct | |
| | ggatcctgtcgaggacacactggaagtagaacttggaaagcctttaactattagctgcaaagcacgatttggctttgaaa | |
| | gggtctttaaccctgtcataaaatggtacatcaaagattctgacctagagtgggaagtctcagtacctgaggcgaaaagt | |
| | attaaatccactttaaaggatgaaatcattgagcgtaatatcatcttggaaaaagtcactcagcgtgatcttcgcaggaagt | |
| | ttgtttgctttgtccagaactccattggaaacacaacccagtccgtccaactgaaagaaaagagaggagtggtgctcctg | |
| | tacatcctgcttggcaccatcgggaccctggtggccgtgctggcggcgagtgccctcctctacaggcactggattgaa | |
| | atagtgctgctgtaccggacctaccagagcaaggatcagacgcttggggataaaaaggattttgatgctttcgtatcctat | |
| | gcaaaatggagctcttttccaagtgaggccacttcatctctgagtgaagaacacttggccctgagcctatttcctgatgttt | |
| | tagaaaacaaatatggatatagcctgtgtttgcttgaaagagatgtggctccaggaggagtgtatgcagaagacattgtg | |
| | agcattattaagagaagcagaagaggaatatttatcttgagccccaactatgtcaatggacccagtatctttgaactacaa | |
| | gcagcagtgaatcttgccttggatgatcaaacactgaaactcattttaattaagttctgttacttccaagagccagagtctct | |
| | acctcatctcgtgaaaaaagctctcagggttttgcccacagttacttggagaggcttaaaatcagttcctcccaattctagg | |
| | ttctgggccaaaatgcgctaccacatgcctgtgaaaaactctcagggattcacgtggaaccagctcagaattacctctag | |
| | gatttttcagtggaaaggactcagtagaacagaaaccactgggaggagctcccagcctaaggaatggtgataa | |
| CAR C2 CD28- | atggccttaccagtgaccgccttgctcctgccgctggccttgctgctccacgccgccaggccggaggtgcagctggtg | 136 |
| CD28-CD28CD3 | gagtctgggggaggcctggtcaagcctggggggtccctgagactctcctgtgcagcctctggattcaccttcagtggc | |
| 6XFOXP3NFAT | tatgccatgagctgggtccgccaggctccagggaaggggctggagtgggtctcaaccattagtagtggcggaaccta | |
| mCMV | catatactaccccgactcagtgaagggccgattcaccatctccagagacaacgccaagaactcactgtatctgcaaatg | |
| IL18Ralpha-T2A- | aacagcctgagagccgaggacacggccgtgtattactgtgcgagacttgggggggataattactacgaatacttcgat | |
| IL18Rb | gtctggggcaaagggaccacggtcaccgtctcctccggcggtggcggatccggcggtggcggatccggcggtggc | |
| | ggatccgacattgtgctgacccagtctccagcctccttggccgtgtctccaggacagagggccaccatcacctgcaga | |
| | gccagtaagagtgtcagtaccagcggatactcctacatgcactggtatcagcagaaaccaggacaacctcctaaactc | |
| | ctgatttacctggcatccaatctggagagcggggtcccagccaggttcagcggcagtgggtctgggaccgatttcacc | |
| | ctcacaattaatcctgtggaagctaatgatactgcaaattattactgtcagcacagtagggagctgcctttcacattcggc | |
| | ggagggaccaaggtggagatcaaacgaactaagcacctgtgtccttctccactgttccccggccctagcaagcctttct | |
| | gggtcctggtggtggtgggggagtgctggcctgctacagcctgctggtgaccgtggcctttatcatcttctgggtgag | |
| | gagtaagaggagcaggctcctgcacagtgactacatgaacatgactcctagaagacctgggcctaccagaaagcatt | |
| | accagccctatgccccaccacgcgacttcgcagcctatcgctccagagtgaagttcagcaggagcgcagacgcccc | |
| | cgcgtacaagcagggccagaaccagctctataacgagctcaatctaggacgaagagaggagtacgatgttttggaca | |
| | agagacgtggccgggaccctgagatggggggaaagccgagaaggaagaaccctcaggaaggcctgtacaatgaa | |
| | ctgcagaaagataagatggcggaggcctacagtgagattgggatgaaaggcgagcgccggaggggcaaggggca | |
| | cgatggcctttaccagggtctcagtacagccaccaaggacacctacgacgcccttcacatgcaggccctgccccctcg | |
| | ctgataagtttaaactgccagaacatttctctggcctaactggccggtaccggcttcattttttccatttactgcagaggctt | |
| | cattttttccatttactgcagaggcttcattttttccatttactgcagaggcttcattttttccatttactgcagaggcttcatttttt | |
| | ccatttactgcagaggcttcattttttccatttactgcagaactagttaggcgtgtacggtgggaggcctatataagcagag | |
| | ctcgtttagtgaaccgtcagatcgcctggagacgccatccacgctgttttgacctccatagaagacaccgggaccgatc | |
| | cagcctcgagagacccaatgctagccaccatgaattgtagagaattacccttgaccctttgggtgcttatatctgtaagc | |
| | actgcagaatcttgtacttcacgtccccacattactgtggttgaaggggaacctttctatctgaaacattgctcgtgttcact | |
| | tgcacatgagattgaaacaaccaccaaaagctggtacaaaagcagtggatcacaggaacatgtggagctgaacccaa | |
| | ggagttccagcagaattgctttgcatgattgtgttttggagttttggccagttgagttgaatgacacaggatcttactttttcc | |
| | aaatgaaaaattatactcagaaatggaaattaaatgtcatcagaagaaataaacacagctgtttcactgaaagacaagta | |
| | actagtaaaattgtggaagttaaaaaattttttcagataacctgtgaaaacagttactatcaaacactggtcaacagcacat | |
| | cattgtataagaactgtaaaaagctactactggagaacaataaaaacccaacgataaagaagaacgccgagtttgaag | |
| | atcaggggtattactcctgcgtgcatttccttcatcataatggaaaactatttaatatcaccaaaaccttcaatataacaata | |
| | gtggaagatcgcagtaatatagttccggttcttcttggaccaaagcttaaccatgttgcagtggaattaggaaaaaacgta | |
| | aggctcaactgctctgctttgctgaatgaagaggatgtaatttattggatgttcggggaagaaaatggatcggatcctaat | |
| | atacatgaagagaaagaaatgagaattatgactccagaaggcaaatggcatgcttcaaaagtattgagaattgaaaatat | |
| | tggtgaaagcaatctaaatgttttatataattgcactgtggccagcacgggaggcacagacaccaaaagcttcatcttgg | |
| | tgagaaaagcagacatggctgatatcccaggccacgtcttcacaagaggaatgatcatagctgttttgatcttggtggca | |
| | gtagtgtgcctagtgactgtgtgtgtcatttatagagttgacttggttctattttatagacatttaacgagaagagatgaaac | |
| | attaacagatggaaaaacatatgatgcttttgtgtcttacctaaaagaatgccgacctgaaaatggagaggagcacacct | |
| | ttgctgtggagattttgcccagggtgttggagaaacattttgggtataagttatgcatatttgaaagggatgtagtgcctgg | |
| | aggagctgttgttgatgaaatccactcactgatagagaaaagccgaagactaatcattgtcctaagtaaaagttatatgtc | |
| | taatgaggtcaggtatgaacttgaaagtggactccatgaagcattggtggaaagaaaaattaaaataatcttaattgaattt | |
| | acacctgttactgacttcacattcttgccccaatcactaaagcttttgaaatctcacagagttctgaagtggaaggccgata | |
| | aatctctttcttataactcaaggttctggaagaaccttctttacttaatgcctgcaaaaacagtcaagccaggtagagacga | |
| | accggaagtcttgcctgttctttccgagtctggaagcggtgagggaagaggtagcctgctcacgtgcggtgatgtgga | |
| | ggagaaccctgggcctatgctctgtttgggctggatatttctttggcttgttgcaggagagcgaattaaaggatttaatattt | |
| | caggttgttccacaaaaaaactcctttggacatattctacaaggagtgaagaggaatttgtcttattttgtgatttaccagag | |
| | ccacagaaatcacatttctgccacagaaatcgactctcaccaaaacaagtccctgagcacctgcccttcatgggtagta | |
| | acgacctatctgatgtccaatggtaccaacaaccttcgaatggagatccattagaggacattaggaaaagctatcctcac | |
| | atcattcaggacaaatgtacccttcactttttgaccccaggggtgaataattctgggtcatatatttgtagacccaagatgat | |
| | taagagcccctatgatgtagcctgttgtgtcaagatgattttagaagttaagccccagacaaatgcatcctgtgagtattcc | |
| | gcatcacataagcaagacctacttcttgggagcactggctctatttcttgccccagtctcagctgccaaagtgatgcaca | |
| | aagtccagcggtaacctggtacaagaatggaaaactcctctctgtggaaaggagcaaccgaatcgtagtggatgaagt | |
| | ttatgactatcaccagggcacatatgtatgtgattacactcagtcggatactgtgagttcgtggacagtcagagctgttgtt | |
| | caagtgagaaccattgtgggagacactaaactcaaaccagatattctggatcctgtcgaggacacactggaagtagaa | |
| | cttggaaagcctttaactattagctgcaaagcacgatttggctttgaaagggtctttaaccctgtcataaaatggtacatca | |
| | aagattctgacctagagtgggaagtctcagtacctgaggcgaaaagtattaaatccactttaaaggatgaaatcattgag | |
| | cgtaatatcatcttggaaaaagtcactcagcgtgatcttcgcaggaagtttgtttgctttgtccagaactccattggaaaca | |
| | caacccagtccgtccaactgaaagaaaagagaggagtggtgctcctgtacatcctgcttggcaccatcgggaccctgg | |
| | tggccgtgctggcggcgagtgccctcctctacaggcactggattgaaatagtgctgctgtaccggacctaccagagca | |
| | aggatcagacgcttggggataaaaaggattttgatgctttcgtatcctatgcaaaatggagctcttttccaagtgaggcca | |
| | cttcatctctgagtgaagaacacttggccctgagcctatttcctgatgttttagaaaacaaatatggatatagcctgtgtttg | |
| | cttgaaagagatgtggctccaggaggagtgtatgcagaagacattgtgagcattattaagagaagcagaagaggaata | |
| | tttatcttgagccccaactatgtcaatggacccagtatctttgaactacaagcagcagtgaatcttgccttggatgatcaaa | |
| | cactgaaactcattttaattaagttctgttacttccaagagccagagtctctacctcatctcgtgaaaaaagctctcagggtt | |
| | ttgcccacagttacttggagaggcttaaaatcagttcctcccaattctaggttctgggccaaaatgcgctaccacatgcct | |
| | gtgaaaaactctcagggattcacgtggaaccagctcagaattacctctaggatttttcagtggaaaggactcagtagaac | |
| | agaaaccactgggaggagctcccagcctaaggaatggtgataa | |
| CAR C2 CD28- | atggccttaccagtgaccgccttgctcctgccgctggccttgctgctccacgccgccaggccggaggtgcagctggtg | 137 |
| CD28-CD28CD3 | gagtctgggggaggcctggtcaagcctggggggtccctgagactctcctgtgcagcctctggattcaccttcagtggc | |
| 3XFOXP3NFAT | tatgccatgagctgggtccgccaggctccagggaaggggctggagtgggtctcaaccattagtagtggcggaaccta | |
| mCMV | catatactaccccgactcagtgaagggccgattcaccatctccagagacaacgccaagaactcactgtatctgcaaatg | |
| IL18Ralpha-T2A- | aacagcctgagagccgaggacacggccgtgtattactgtgcgagacttgggggggataattactacgaatacttcgat | |
| IL18Rb | gtctggggcaaagggaccacggtcaccgtctcctccggcggtggcggatccggcggtggcggatccggcggtggc | |
| | ggatccgacattgtgctgacccagtctccagcctccttggccgtgtctccaggacagagggccaccatcacctgcaga | |
| | gccagtaagagtgtcagtaccagcggatactcctacatgcactggtatcagcagaaaccaggacaacctcctaaactc | |
| | ctgatttacctggcatccaatctggagagcggggtcccagccaggttcagcggcagtgggtctgggaccgatttcacc | |
| | ctcacaattaatcctgtggaagctaatgatactgcaaattattactgtcagcacagtagggagctgcctttcacattcggc | |
| | ggagggaccaaggtggagatcaaacgaactaagcacctgtgtccttctccactgttccccggccctagcaagcctttct | |
| | gggtcctggtggtggtgggcggagtgctggcctgctacagcctgctggtgaccgtggcctttatcatcttctgggtgag | |
| | gagtaagaggagcaggctcctgcacagtgactacatgaacatgactcctagaagacctgggcctaccagaaagcatt | |
| | accagccctatgccccaccacgcgacttcgcagcctatcgctccagagtgaagttcagcaggagcgcagacgcccc | |
| | cgcgtacaagcagggccagaaccagctctataacgagctcaatctaggacgaagagaggagtacgatgttttggaca | |
| | agagacgtggccgggaccctgagatggggggaaagccgagaaggaagaaccctcaggaaggcctgtacaatgaa | |
| | ctgcagaaagataagatggcggaggcctacagtgagattgggatgaaaggcgagcgccggaggggcaaggggca | |
| | cgatggcctttaccagggtctcagtacagccaccaaggacacctacgacgcccttcacatgcaggccctgccccctcg | |
| | ctgataagtttaaactgccagaacatttctctggcctaactggccggtaccggcttcattttttccatttactgcagaggctt | |
| | cattttttccatttactgcagaggcttcattttttccatttactgcagaactagttaggcgtgtacggtgggaggcctatataa | |
| | gcagagctcgtttagtgaaccgtcagatcgcctggagacgccatccacgctgttttgacctccatagaagacaccggg | |
| | accgatccagcctcgagagacccaatgctagccaccatgaattgtagagaattacccttgaccctttgggtgcttatatc | |
| | tgtaagcactgcagaatcttgtacttcacgtccccacattactgtggttgaaggggaacctttctatctgaaacattgctcgt | |
| | gttcacttgcacatgagattgaaacaaccaccaaaagctggtacaaaagcagtggatcacaggaacatgtggagctga | |
| | acccaaggagttccagcagaattgctttgcatgattgtgttttggagttttggccagttgagttgaatgacacaggatctta | |
| | ctttttccaaatgaaaaattatactcagaaatggaaattaaatgtcatcagaagaaataaacacagctgtttcactgaaaga | |
| | caagtaactagtaaaattgtggaagttaaaaaattttttcagataacctgtgaaaacagttactatcaaacactggtcaaca | |
| | gcacatcattgtataagaactgtaaaaagctactactggagaacaataaaaacccaacgataaagaagaacgccgagt | |
| | ttgaagatcaggggtattactcctgcgtgcatttccttcatcataatggaaaactatttaatatcaccaaaaccttcaatataa | |
| | caatagtggaagatcgcagtaatatagttccggttcttcttggaccaaagcttaaccatgttgcagtggaattaggaaaaa | |
| | acgtaaggctcaactgctctgctttgctgaatgaagaggatgtaatttattggatgttcggggaagaaaatggatcggat | |
| | cctaatatacatgaagagaaagaaatgagaattatgactccagaaggcaaatggcatgcttcaaaagtattgagaattga | |
| | aaatattggtgaaagcaatctaaatgttttatataattgcactgtggccagcacgggaggcacagacaccaaaagcttca | |
| | tcttggtgagaaaagcagacatggctgatatcccaggccacgtcttcacaagaggaatgatcatagctgttttgatcttgg | |
| | tggcagtagtgtgcctagtgactgtgtgtgtcatttatagagttgacttggttctattttatagacatttaacgagaagagatg | |
| | aaacattaacagatggaaaaacatatgatgcttttgtgtcttacctaaaagaatgccgacctgaaaatggagaggagcac | |
| | acctttgctgtggagattttgcccagggtgttggagaaacattttgggtataagttatgcatatttgaaagggatgtagtgc | |
| | ctggaggagctgttgttgatgaaatccactcactgatagagaaaagccgaagactaatcattgtcctaagtaaaagttata | |
| | tgtctaatgaggtcaggtatgaacttgaaagtggactccatgaagcattggtggaaagaaaaattaaaataatcttaattg | |
| | aatttacacctgttactgacttcacattcttgccccaatcactaaagcttttgaaatctcacagagttctgaagtggaaggcc | |
| | gataaatctctttcttataactcaaggttctggaagaaccttctttacttaatgcctgcaaaaacagtcaagccaggtagag | |
| | acgaaccggaagtcttgcctgttctttccgagtctggaagcggtgagggaagaggtagcctgctcacgtgcggtgatg | |
| | tggaggagaaccctgggcctatgctctgtttgggctggatatttctttggcttgttgcaggagagcgaattaaaggattta | |
| | atatttcaggttgttccacaaaaaaactcctttggacatattctacaaggagtgaagaggaatttgtcttattttgtgatttacc | |
| | agagccacagaaatcacatttctgccacagaaatcgactctcaccaaaacaagtccctgagcacctgcccttcatgggt | |
| | agtaacgacctatctgatgtccaatggtaccaacaaccttcgaatggagatccattagaggacattaggaaaagctatcc | |
| | tcacatcattcaggacaaatgtacccttcactttttgaccccaggggtgaataattctgggtcatatatttgtagacccaag | |
| | atgattaagagcccctatgatgtagcctgttgtgtcaagatgattttagaagttaagccccagacaaatgcatcctgtgagt | |
| | attccgcatcacataagcaagacctacttcttgggagcactggctctatttcttgccccagtctcagctgccaaagtgatg | |
| | cacaaagtccagcggtaacctggtacaagaatggaaaactcctctctgtggaaaggagcaaccgaatcgtagtggatg | |
| | aagtttatgactatcaccagggcacatatgtatgtgattacactcagtcggatactgtgagttcgtggacagtcagagctg | |
| | ttgttcaagtgagaaccattgtgggagacactaaactcaaaccagatattctggatcctgtcgaggacacactggaagta | |
| | gaacttggaaagcctttaactattagctgcaaagcacgatttggctttgaaagggtctttaaccctgtcataaaatggtaca | |
| | tcaaagattctgacctagagtgggaagtctcagtacctgaggcgaaaagtattaaatccactttaaaggatgaaatcattg | |
| | agcgtaatatcatcttggaaaaagtcactcagcgtgatcttcgcaggaagtttgtttgctttgtccagaactccattggaaa | |
| | cacaacccagtccgtccaactgaaagaaaagagaggagtggtgctcctgtacatcctgcttggcaccatcgggaccct | |
| | ggtggccgtgctggcggcgagtgccctcctctacaggcactggattgaaatagtgctgctgtaccggacctaccagag | |
| | caaggatcagacgcttggggataaaaaggattttgatgctttcgtatcctatgcaaaatggagctcttttccaagtgaggc | |
| | cacttcatctctgagtgaagaacacttggccctgagcctatttcctgatgttttagaaaacaaatatggatatagcctgtgtt | |
| | tgcttgaaagagatgtggctccaggaggagtgtatgcagaagacattgtgagcattattaagagaagcagaagaggaa | |
| | tatttatcttgagccccaactatgtcaatggacccagtatctttgaactacaagcagcagtgaatcttgccttggatgatca | |
| | aacactgaaactcattttaattaagttctgttacttccaagagccagagtctctacctcatctcgtgaaaaaagctctcagg | |
| | gttttgcccacagttacttggagaggcttaaaatcagttcctcccaattctaggttctgggccaaaatgcgctaccacatg | |
| | cctgtgaaaaactctcagggattcacgtggaaccagctcagaattacctctaggatttttcagtggaaaggactcagtag | |
| | aacagaaaccactgggaggagctcccagcctaaggaatggtgataa | |
| CAR C2 CD28- | atggccttaccagtgaccgccttgctcctgccgctggccttgctgctccacgccgccaggccggaggtgcagctggtg | 138 |
| CD28-CD28CD3 | gagtctgggggaggcctggtcaagcctggggggtccctgagactctcctgtgcagcctctggattcaccttcagtggc | |
| 6XIL2NFAT | tatgccatgagctgggtccgccaggctccagggaaggggctggagtgggtctcaaccattagtagtggcggaaccta | |
| mCMV | catatactaccccgactcagtgaagggccgattcaccatctccagagacaacgccaagaactcactgtatctgcaaatg | |
| IL18Ralpha-T2A- | aacagcctgagagccgaggacacggccgtgtattactgtgcgagacttgggggggataattactacgaatacttcgat | |
| IL18Rb | gtctggggcaaagggaccacggtcaccgtctcctccggcggtggcggatccggcggtggcggatccggcggtggc | |
| | ggatccgacattgtgctgacccagtctccagcctccttggccgtgtctccaggacagagggccaccatcacctgcaga | |
| | gccagtaagagtgtcagtaccagcggatactcctacatgcactggtatcagcagaaaccaggacaacctcctaaactc | |
| | ctgatttacctggcatccaatctggagagcggggtcccagccaggttcagcggcagtgggtctgggaccgatttcacc | |
| | ctcacaattaatcctgtggaagctaatgatactgcaaattattactgtcagcacagtagggagctgcctttcacattcggc | |
| | ggagggaccaaggtggagatcaaacgaactaagcacctgtgtccttctccactgttccccggccctagcaagcctttct | |
| | gggtcctggtggtggtgggcggagtgctggcctgctacagcctgctggtgaccgtggcctttatcatcttctgggtgag | |
| | gagtaagaggagcaggctcctgcacagtgactacatgaacatgactcctagaagacctgggcctaccagaaagcatt | |
| | accagccctatgccccaccacgcgacttcgcagcctatcgctccagagtgaagttcagcaggagcgcagacgcccc | |
| | cgcgtacaagcagggccagaaccagctctataacgagctcaatctaggacgaagagaggagtacgatgttttggaca | |
| | agagacgtggccgggaccctgagatggggggaaagccgagaaggaagaaccctcaggaaggcctgtacaatgaa | |
| | ctgcagaaagataagatggcggaggcctacagtgagattgggatgaaaggcgagcgccggaggggcaaggggca | |
| | cgatggcctttaccagggtctcagtacagccaccaaggacacctacgacgcccttcacatgcaggccctgccccctcg | |
| | ctgataagtttaaactgccagaacatttctctggcctaactggccggtaccggaggaaaaactgtttcatacagaaggcg | |
| | tggaggaaaaactgtttcatacagaaggcgtggaggaaaaactgtttcatacagaaggcgtggaggaaaaactgtttc | |
| | atacagaaggcgtggaggaaaaactgtttcatacagaaggcgtggaggaaaaactgtttcatacagaaggcgtactag | |
| | ttaggcgtgtacggtgggaggcctatataagcagagctcgtttagtgaaccgtcagatcgcctggagacgccatccac | |
| | gctgttttgacctccatagaagacaccgggaccgatccagcctcgagagacccaatgctagccaccatgaattgtaga | |
| | gaattacccttgaccctttgggtgcttatatctgtaagcactgcagaatcttgtacttcacgtccccacattactgtggttga | |
| | aggggaacctttctatctgaaacattgctcgtgttcacttgcacatgagattgaaacaaccaccaaaagctggtacaaaa | |
| | gcagtggatcacaggaacatgtggagctgaacccaaggagttccagcagaattgctttgcatgattgtgttttggagtttt | |
| | ggccagttgagttgaatgacacaggatcttactttttccaaatgaaaaattatactcagaaatggaaattaaatgtcatcag | |
| | aagaaataaacacagctgtttcactgaaagacaagtaactagtaaaattgtggaagttaaaaaattttttcagataacctgt | |
| | gaaaacagttactatcaaacactggtcaacagcacatcattgtataagaactgtaaaaagctactactggagaacaataa | |
| | aaacccaacgataaagaagaacgccgagtttgaagatcaggggtattactcctgcgtgcatttccttcatcataatggaa | |
| | aactatttaatatcaccaaaaccttcaatataacaatagtggaagatcgcagtaatatagttccggttcttcttggaccaaa | |
| | gcttaaccatgttgcagtggaattaggaaaaaacgtaaggctcaactgctctgctttgctgaatgaagaggatgtaattta | |
| | ttggatgttcggggaagaaaatggatcggatcctaatatacatgaagagaaagaaatgagaattatgactccagaaggc | |
| | aaatggcatgcttcaaaagtattgagaattgaaaatattggtgaaagcaatctaaatgttttatataattgcactgtggccag | |
| | cacgggaggcacagacaccaaaagcttcatcttggtgagaaaagcagacatggctgatatcccaggccacgtcttca | |
| | caagaggaatgatcatagctgttttgatcttggtggcagtagtgtgcctagtgactgtgtgtgtcatttatagagttgacttg | |
| | gttctattttatagacatttaacgagaagagatgaaacattaacagatggaaaaacatatgatgcttttgtgtcttacctaaa | |
| | agaatgccgacctgaaaatggagaggagcacacctttgctgtggagattttgcccagggtgttggagaaacattttggg | |
| | tataagttatgcatatttgaaagggatgtagtgcctggaggagctgttgttgatgaaatccactcactgatagagaaaagc | |
| | cgaagactaatcattgtcctaagtaaaagttatatgtctaatgaggtcaggtatgaacttgaaagtggactccatgaagca | |
| | ttggtggaaagaaaaattaaaataatcttaattgaatttacacctgttactgacttcacattcttgccccaatcactaaagcttt | |
| | tgaaatctcacagagttctgaagtggaaggccgataaatctctttcttataactcaaggttctggaagaaccttctttactta | |
| | atgcctgcaaaaacagtcaagccaggtagagacgaaccggaagtcttgcctgttctttccgagtctggaagcggtgag | |
| | ggaagaggtagcctgctcacgtgcggtgatgtggaggagaaccctgggcctatgctctgtttgggctggatatttctttg | |
| | gcttgttgcaggagagcgaattaaaggatttaatatttcaggttgttccacaaaaaaactcctttggacatattctacaagg | |
| | agtgaagaggaatttgtcttattttgtgatttaccagagccacagaaatcacatttctgccacagaaatcgactctcaccaa | |
| | aacaagtccctgagcacctgcccttcatgggtagtaacgacctatctgatgtccaatggtaccaacaaccttcgaatgga | |
| | gatccattagaggacattaggaaaagctatcctcacatcattcaggacaaatgtacccttcactttttgaccccaggggtg | |
| | aataattctgggtcatatatttgtagacccaagatgattaagagcccctatgatgtagcctgttgtgtcaagatgattttaga | |
| | agttaagccccagacaaatgcatcctgtgagtattccgcatcacataagcaagacctacttcttgggagcactggctcta | |
| | tttcttgccccagtctcagctgccaaagtgatgcacaaagtccagcggtaacctggtacaagaatggaaaactcctctct | |
| | gtggaaaggagcaaccgaatcgtagtggatgaagtttatgactatcaccagggcacatatgtatgtgattacactcagtc | |
| | ggatactgtgagttcgtggacagtcagagctgttgttcaagtgagaaccattgtgggagacactaaactcaaaccagat | |
| | attctggatcctgtcgaggacacactggaagtagaacttggaaagcctttaactattagctgcaaagcacgatttggcttt | |
| | gaaagggtctttaaccctgtcataaaatggtacatcaaagattctgacctagagtgggaagtctcagtacctgaggcgaa | |
| | aagtattaaatccactttaaaggatgaaatcattgagcgtaatatcatcttggaaaaagtcactcagcgtgatcttcgcagg | |
| | aagtttgtttgctttgtccagaactccattggaaacacaacccagtccgtccaactgaaagaaaagagaggagtggtgct | |
| | cctgtacatcctgcttggcaccatcgggaccctggtggccgtgctggggcgagtgccctcctctacaggcactggatt | |
| | gaaatagtgctgctgtaccggacctaccagagcaaggatcagacgcttggggataaaaaggattttgatgctttcgtatc | |
| | ctatgcaaaatggagctcttttccaagtgaggccacttcatctctgagtgaagaacacttggccctgagcctatttcctgat | |
| | gttttagaaaacaaatatggatatagcctgtgtttgcttgaaagagatgtggctccaggaggagtgtatgcagaagacatt | |
| | gtgagcattattaagagaagcagaagaggaatatttatcttgagccccaactatgtcaatggacccagtatctttgaacta | |
| | caagcagcagtgaatcttgccttggatgatcaaacactgaaactcattttaattaagttctgttacttccaagagccagagt | |
| | ctctacctcatctcgtgaaaaaagctctcagggttttgcccacagttacttggagaggcttaaaatcagttcctcccaattct | |
| | aggttctgggccaaaatgcgctaccacatgcctgtgaaaaactctcagggattcacgtggaaccagctcagaattacct | |
| | ctaggatttttcagtggaaaggactcagtagaacagaaaccactgggaggagctcccagcctaaggaatggtgataa | |
| CAR C2 CD28- | atggccttaccagtgaccgccttgctcctgccgctggccttgctgctccacgccgccaggccggaggtgcagctggtg | 139 |
| CD28-CD28CD3 | gagtctgggggaggcctggtcaagcctggggggtccctgagactctcctgtgcagcctctggattcaccttcagtggc | |
| 3XIL2NFAT | tatgccatgagctgggtccgccaggctccagggaaggggctggagtgggtctcaaccattagtagtggcggaaccta | |
| mCMV | catatactaccccgactcagtgaagggccgattcaccatctccagagacaacgccaagaactcactgtatctgcaaatg | |
| IL18Ralpha-T2A- | aacagcctgagagccgaggacacggccgtgtattactgtgcgagacttgggggggataattactacgaatacttcgat | |
| IL18Rb | gtctggggcaaagggaccacggtcaccgtctcctccggcggtggcggatccggcggtggcggatccggcggtggc | |
| | ggatccgacattgtgctgacccagtctccagcctccttggccgtgtctccaggacagagggccaccatcacctgcaga | |
| | gccagtaagagtgtcagtaccagcggatactcctacatgcactggtatcagcagaaaccaggacaacctcctaaactc | |
| | ctgatttacctggcatccaatctggagagcggggtcccagccaggttcagcggcagtgggtctgggaccgatttcacc | |
| | ctcacaattaatcctgtggaagctaatgatactgcaaattattactgtcagcacagtagggagctgcctttcacattcggc | |
| | ggagggaccaaggtggagatcaaacgaactaagcacctgtgtccttctccactgttccccggccctagcaagcctttct | |
| | gggtcctggtggtggtgggcggagtgctggcctgctacagcctgctggtgaccgtggcctttatcatcttctgggtgag | |
| | gagtaagaggagcaggctcctgcacagtgactacatgaacatgactcctagaagacctgggcctaccagaaagcatt | |
| | accagccctatgccccaccacgcgacttcgcagcctatcgctccagagtgaagttcagcaggagcgcagacgcccc | |
| | cgcgtacaagcagggccagaaccagctctataacgagctcaatctaggacgaagagaggagtacgatgttttggaca | |
| | agagacgtggccgggaccctgagatggggggaaagccgagaaggaagaaccctcaggaaggcctgtacaatgaa | |
| | ctgcagaaagataagatggcggaggcctacagtgagattgggatgaaaggcgagcgccggaggggcaaggggca | |
| | cgatggcctttaccagggtctcagtacagccaccaaggacacctacgacgcccttcacatgcaggccctgccccctcg | |
| | ctgataagtttaaactgccagaacatttctctggcctaactggccggtaccggaggaaaaactgtttcatacagaaggcg | |
| | tggaggaaaaactgtttcatacagaaggcgtggaggaaaaactgtttcatacagaaggcgtactagttaggcgtgtacg | |
| | gtgggaggcctatataagcagagctcgtttagtgaaccgtcagatcgcctggagacgccatccacgctgttttgacctc | |
| | catagaagacaccgggaccgatccagcctcgagagacccaatgctagccaccatgaattgtagagaattacccttga | |
| | ccctttgggtgcttatatctgtaagcactgcagaatcttgtacttcacgtccccacattactgtggttgaaggggaacctttc | |
| | tatctgaaacattgctcgtgttcacttgcacatgagattgaaacaaccaccaaaagctggtacaaaagcagtggatcaca | |
| | ggaacatgtggagctgaacccaaggagttccagcagaattgctttgcatgattgtgttttggagttttggccagttgagtt | |
| | gaatgacacaggatcttactttttccaaatgaaaaattatactcagaaatggaaattaaatgtcatcagaagaaataaaca | |
| | cagctgtttcactgaaagacaagtaactagtaaaattgtggaagttaaaaaattttttcagataacctgtgaaaacagttact | |
| | atcaaacactggtcaacagcacatcattgtataagaactgtaaaaagctactactggagaacaataaaaacccaacgat | |
| | aaagaagaacgccgagtttgaagatcaggggtattactcctgcgtgcatttccttcatcataatggaaaactatttaatatc | |
| | accaaaaccttcaatataacaatagtggaagatcgcagtaatatagttccggttcttcttggaccaaagcttaaccatgttg | |
| | cagtggaattaggaaaaaacgtaaggctcaactgctctgctttgctgaatgaagaggatgtaatttattggatgttcggg | |
| | gaagaaaatggatcggatcctaatatacatgaagagaaagaaatgagaattatgactccagaaggcaaatggcatgctt | |
| | caaaagtattgagaattgaaaatattggtgaaagcaatctaaatgttttatataattgcactgtggccagcacgggaggca | |
| | cagacaccaaaagcttcatcttggtgagaaaagcagacatggctgatatcccaggccacgtcttcacaagaggaatga | |
| | tcatagctgttttgatcttggtggcagtagtgtgcctagtgactgtgtgtgtcatttatagagttgacttggttctattttataga | |
| | catttaacgagaagagatgaaacattaacagatggaaaaacatatgatgcttttgtgtcttacctaaaagaatgccgacct | |
| | gaaaatggagaggagcacacctttgctgtggagattttgcccagggtgttggagaaacattttgggtataagttatgcat | |
| | atttgaaagggatgtagtgcctggaggagctgttgttgatgaaatccactcactgatagagaaaagccgaagactaatc | |
| | attgtcctaagtaaaagttatatgtctaatgaggtcaggtatgaacttgaaagtggactccatgaagcattggtggaaaga | |
| | aaaattaaaataatcttaattgaatttacacctgttactgacttcacattcttgccccaatcactaaagcttttgaaatctcaca | |
| | gagttctgaagtggaaggccgataaatctctttcttataactcaaggttctggaagaaccttctttacttaatgcctgcaaaa | |
| | acagtcaagccaggtagagacgaaccggaagtcttgcctgttctttccgagtctggaagcggtgagggaagaggtag | |
| | cctgctcacgtgcggtgatgtggaggagaaccctgggcctatgctctgtttgggctggatatttctttggcttgttgcagg | |
| | agagcgaattaaaggatttaatatttcaggttgttccacaaaaaaactcctttggacatattctacaaggagtgaagagga | |
| | atttgtcttattttgtgatttaccagagccacagaaatcacatttctgccacagaaatcgactctcaccaaaacaagtccctg | |
| | agcacctgcccttcatgggtagtaacgacctatctgatgtccaatggtaccaacaaccttcgaatggagatccattagag | |
| | gacattaggaaaagctatcctcacatcattcaggacaaatgtacccttcactttttgaccccaggggtgaataattctgggt | |
| | catatatttgtagacccaagatgattaagagcccctatgatgtagcctgttgtgtcaagatgattttagaagttaagcccca | |
| | gacaaatgcatcctgtgagtattccgcatcacataagcaagacctacttcttgggagcactggctctatttcttgccccagt | |
| | ctcagctgccaaagtgatgcacaaagtccagcggtaacctggtacaagaatggaaaactcctctctgtggaaaggagc | |
| | aaccgaatcgtagtggatgaagtttatgactatcaccagggcacatatgtatgtgattacactcagtcggatactgtgagt | |
| | tcgtggacagtcagagctgttgttcaagtgagaaccattgtgggagacactaaactcaaaccagatattctggatcctgt | |
| | cgaggacacactggaagtagaacttggaaagcctttaactattagctgcaaagcacgatttggctttgaaagggtcttta | |
| | accctgtcataaaatggtacatcaaagattctgacctagagtgggaagtctcagtacctgaggcgaaaagtattaaatcc | |
| | actttaaaggatgaaatcattgagcgtaatatcatcttggaaaaagtcactcagcgtgatcttcgcaggaagtttgtttgctt | |
| | tgtccagaactccattggaaacacaacccagtccgtccaactgaaagaaaagagaggagtggtgctcctgtacatcct | |
| | gcttggcaccatcgggaccctggtggccgtgctggcggcgagtgccctcctctacaggcactggattgaaatagtgct | |
| | gctgtaccggacctaccagagcaaggatcagacgcttggggataaaaaggattttgatgctttcgtatcctatgcaaaat | |
| | ggagctcttttccaagtgaggccacttcatctctgagtgaagaacacttggccctgagcctatttcctgatgttttagaaaa | |
| | caaatatggatatagcctgtgtttgcttgaaagagatgtggctccaggaggagtgtatgcagaagacattgtgagcatta | |
| | ttaagagaagcagaagaggaatatttatcttgagccccaactatgtcaatggacccagtatctttgaactacaagcagca | |
| | gtgaatcttgccttggatgatcaaacactgaaactcattttaattaagttctgttacttccaagagccagagtctctacctcat | |
| | ctcgtgaaaaaagctctcagggttttgcccacagttacttggagaggcttaaaatcagttcctcccaattctaggttctggg | |
| | ccaaaatgcgctaccacatgcctgtgaaaaactctcagggattcacgtggaaccagctcagaattacctctaggatttttc | |
| | agtggaaaggactcagtagaacagaaaccactgggaggagctcccagcctaaggaatggtgataa | |
| CAR C2 CD28- | atggccttaccagtgaccgccttgctcctgccgctggccttgctgctccacgccgccaggccggaggtgcagctggtg | 140 |
| CD28-CD281XX | gagtctgggggaggcctggtcaagcctggggggtccctgagactctcctgtgcagcctctggattcaccttcagtggc | |
| 6XFOXP3NFAT | tatgccatgagctgggtccgccaggctccagggaaggggctggagtgggtctcaaccattagtagtggcggaaccta | |
| mCMV | catatactaccccgactcagtgaagggccgattcaccatctccagagacaacgccaagaactcactgtatctgcaaatg | |
| IL18Ralpha-T2A- | aacagcctgagagccgaggacacggccgtgtattactgtgcgagacttgggggggataattactacgaatacttcgat | |
| IL18Rb | gtctggggcaaagggaccacggtcaccgtctcctccggcggtggcggatccggcggtggcggatccggcggtggc | |
| | ggatccgacattgtgctgacccagtctccagcctccttggccgtgtctccaggacagagggccaccatcacctgcaga | |
| | gccagtaagagtgtcagtaccagcggatactcctacatgcactggtatcagcagaaaccaggacaacctcctaaactc | |
| | ctgatttacctggcatccaatctggagagcggggtcccagccaggttcagcggcagtgggtctgggaccgatttcacc | |
| | ctcacaattaatcctgtggaagctaatgatactgcaaattattactgtcagcacagtagggagctgcctttcacattcggc | |
| | ggagggaccaaggtggagatcaaacgaactaagcacctgtgtccttctccactgttccccggccctagcaagcctttct | |
| | gggtcctggtggtggtgggcggagtgctggcctgctacagcctgctggtgaccgtggcctttatcatcttctgggtgag | |
| | gagtaagaggagcaggctcctgcacagtgactacatgaacatgactcctagaagacctgggcctaccagaaagcatt | |
| | accagccctatgccccaccacgcgacttcgcagcctatcgctccagagtgaagttcagcaggagcgcagacgcccc | |
| | cgcgtacaagcagggccagaaccagctctataacgagctcaatctaggacgaagagaggagtacgatgttttggaca | |
| | agagacgtggccgggaccctgagatggggggaaagccgagaaggaagaaccctcaggaaggcctgttcaatgaa | |
| | ctgcagaaagataagatggcggaggccttcagtgagattgggatgaaaggcgagcgccggaggggcaaggggca | |
| | cgatggccttttccagggtctcagtacagccaccaaggacaccttcgacgcccttcacatgcaggccctgccccctcg | |
| | ctgataagtttaaactgccagaacattactctggcctaactggccggtaccggcttcattttttccatttactgcagaggctt | |
| | cattttttccatttactgcagaggcttcattttttccatttactgcagaggcttcattttttccatttactgcagaggcttcatttttt | |
| | ccatttactgcagaggcttcattttttccatttactgcagaactagttaggcgtgtacggtgggaggcctatataagcagag | |
| | ctcgtttagtgaaccgtcagatcgcctggagacgccatccacgctgttttgacctccatagaagacaccgggaccgatc | |
| | cagcctcgagagacccaatgctagccaccatgaattgtagagaattacccttgaccctttgggtgcttatatctgtaagc | |
| | actgcagaatcttgtacttcacgtccccacattactgtggttgaaggggaacctttctatctgaaacattgctcgtgttcact | |
| | tgcacatgagattgaaacaaccaccaaaagctggtacaaaagcagtggatcacaggaacatgtggagctgaacccaa | |
| | ggagttccagcagaattgctttgcatgattgtgttttggagttttggccagttgagttgaatgacacaggatcttactttttcc | |
| | aaatgaaaaattatactcagaaatggaaattaaatgtcatcagaagaaataaacacagctgtttcactgaaagacaagta | |
| | actagtaaaattgtggaagttaaaaaattttttcagataacctgtgaaaacagttactatcaaacactggtcaacagcacat | |
| | cattgtataagaactgtaaaaagctactactggagaacaataaaaacccaacgataaagaagaacgccgagtttgaag | |
| | atcaggggtattactcctgcgtgcatttccttcatcataatggaaaactatttaatatcaccaaaaccttcaatataacaata | |
| | gtggaagatcgcagtaatatagttccggttcttcttggaccaaagcttaaccatgttgcagtggaattaggaaaaaacgta | |
| | aggctcaactgctctgctttgctgaatgaagaggatgtaatttattggatgttcggggaagaaaatggatcggatcctaat | |
| | atacatgaagagaaagaaatgagaattatgactccagaaggcaaatggcatgcttcaaaagtattgagaattgaaaatat | |
| | tggtgaaagcaatctaaatgttttatataattgcactgtggccagcacgggaggcacagacaccaaaagcttcatcttgg | |
| | tgagaaaagcagacatggctgatatcccaggccacgtcttcacaagaggaatgatcatagctgttttgatcttggtggca | |
| | gtagtgtgcctagtgactgtgtgtgtcatttatagagttgacttggttctattttatagacatttaacgagaagagatgaaac | |
| | attaacagatggaaaaacatatgatgcttttgtgtcttacctaaaagaatgccgacctgaaaatggagaggagcacacct | |
| | ttgctgtggagattttgcccagggtgttggagaaacattttgggtataagttatgcatatttgaaagggatgtagtgcctgg | |
| | aggagctgttgttgatgaaatccactcactgatagagaaaagccgaagactaatcattgtcctaagtaaaagttatatgtc | |
| | taatgaggtcaggtatgaacttgaaagtggactccatgaagcattggtggaaagaaaaattaaaataatcttaattgaattt | |
| | acacctgttactgacttcacattcttgccccaatcactaaagcttttgaaatctcacagagttctgaagtggaaggccgata | |
| | aatctctttcttataactcaaggttctggaagaaccttctttacttaatgcctgcaaaaacagtcaagccaggtagagacga | |
| | accggaagtcttgcctgttctttccgagtctggaagcggtgagggaagaggtagcctgctcacgtgcggtgatgtgga | |
| | ggagaaccctgggcctatgctctgtttgggctggatatttctttggcttgttgcaggagagcgaattaaaggatttaatattt | |
| | caggttgttccacaaaaaaactcctttggacatattctacaaggagtgaagaggaatttgtcttattttgtgatttaccagag | |
| | ccacagaaatcacatttctgccacagaaatcgactctcaccaaaacaagtccctgagcacctgcccttcatgggtagta | |
| | acgacctatctgatgtccaatggtaccaacaaccttcgaatggagatccattagaggacattaggaaaagctatcctcac | |
| | atcattcaggacaaatgtacccttcactttttgaccccaggggtgaataattctgggtcatatatttgtagacccaagatgat | |
| | taagagcccctatgatgtagcctgttgtgtcaagatgattttagaagttaagccccagacaaatgcatcctgtgagtattcc | |
| | gcatcacataagcaagacctacttcttgggagcactggctctatttcttgccccagtctcagctgccaaagtgatgcaca | |
| | aagtccagcggtaacctggtacaagaatggaaaactcctctctgtggaaaggagcaaccgaatcgtagtggatgaagt | |
| | ttatgactatcaccagggcacatatgtatgtgattacactcagtcggatactgtgagttcgtggacagtcagagctgttgtt | |
| | caagtgagaaccattgtgggagacactaaactcaaaccagatattctggatcctgtcgaggacacactggaagtagaa | |
| | cttggaaagcctttaactattagctgcaaagcacgatttggctttgaaagggtctttaaccctgtcataaaatggtacatca | |
| | aagattctgacctagagtgggaagtctcagtacctgaggcgaaaagtattaaatccactttaaaggatgaaatcattgag | |
| | cgtaatatcatcttggaaaaagtcactcagcgtgatcttcgcaggaagtttgtttgctttgtccagaactccattggaaaca | |
| | caacccagtccgtccaactgaaagaaaagagaggagtggtgctcctgtacatcctgcttggcaccatcgggaccctgg | |
| | tggccgtgctggcggcgagtgccctcctctacaggcactggattgaaatagtgctgctgtaccggacctaccagagca | |
| | aggatcagacgcttggggataaaaaggattttgatgctttcgtatcctatgcaaaatggagctcttttccaagtgaggcca | |
| | cttcatctctgagtgaagaacacttggccctgagcctatttcctgatgttttagaaaacaaatatggatatagcctgtgtttg | |
| | cttgaaagagatgtggctccaggaggagtgtatgcagaagacattgtgagcattattaagagaagcagaagaggaata | |
| | tttatcttgagccccaactatgtcaatggacccagtatctttgaactacaagcagcagtgaatcttgccttggatgatcaaa | |
| | cactgaaactcattttaattaagttctgttacttccaagagccagagtctctacctcatctcgtgaaaaaagctctcagggtt | |
| | ttgcccacagttacttggagaggcttaaaatcagttcctcccaattctaggttctgggccaaaatgcgctaccacatgcct | |
| | gtgaaaaactctcagggattcacgtggaaccagctcagaattacctctaggatttttcagtggaaaggactcagtagaac | |
| | agaaaccactgggaggagctcccagcctaaggaatggtgataa | |
| CAR C2 CD28- | atggccttaccagtgaccgccttgctcctgccgctggccttgctgctccacgccgccaggccggaggtgcagctggtg | 141 |
| CD28-CD281XX | gagtctgggggaggcctggtcaagcctggggggtccctgagactctcctgtgcagcctctggattcaccttcagtggc | |
| 3XFOXP3NFAT | tatgccatgagctgggtccgccaggctccagggaaggggctggagtgggtctcaaccattagtagtggcggaaccta | |
| mCMV | catatactaccccgactcagtgaagggccgattcaccatctccagagacaacgccaagaactcactgtatctgcaaatg | |
| IL18Ralpha-T2A- | aacagcctgagagccgaggacacggccgtgtattactgtgcgagacttgggggggataattactacgaatacttcgat | |
| IL18Rb | gtctggggcaaagggaccacggtcaccgtctcctccggcggtggcggatccggcggtggcggatccggcggtggc | |
| | ggatccgacattgtgctgacccagtctccagcctccttggccgtgtctccaggacagagggccaccatcacctgcaga | |
| | gccagtaagagtgtcagtaccagcggatactcctacatgcactggtatcagcagaaaccaggacaacctcctaaactc | |
| | ctgatttacctggcatccaatctggagagcggggtcccagccaggttcagcggcagtgggtctgggaccgatttcacc | |
| | ctcacaattaatcctgtggaagctaatgatactgcaaattattactgtcagcacagtagggagctgcctttcacattcggc | |
| | ggagggaccaaggtggagatcaaacgaactaagcacctgtgtccttctccactgttccccggccctagcaagcctttct | |
| | gggtcctggtggtggtgggcggagtgctggcctgctacagcctgctggtgaccgtggcctttatcatcttctgggtgag | |
| | gagtaagaggagcaggctcctgcacagtgactacatgaacatgactcctagaagacctgggcctaccagaaagcatt | |
| | accagccctatgccccaccacgcgacttcgcagcctatcgctccagagtgaagttcagcaggagcgcagacgcccc | |
| | cgcgtacaagcagggccagaaccagctctataacgagctcaatctaggacgaagagaggagtacgatgttttggaca | |
| | agagacgtggccgggaccctgagatggggggaaagccgagaaggaagaaccctcaggaaggcctgttcaatgaa | |
| | ctgcagaaagataagatggcggaggccttcagtgagattgggatgaaaggcgagcgccggaggggcaaggggca | |
| | cgatggccttttccagggtctcagtacagccaccaaggacaccttcgacgcccttcacatgcaggccctgccccctcg | |
| | ctgataagtttaaactgccagaacattactctggcctaactggccggtaccggcttcattttttccatttactgcagaggctt | |
| | cattttttccatttactgcagaggcttcattttttccatttactgcagaactagttaggcgtgtacggtgggaggcctatataa | |
| | gcagagctcgtttagtgaaccgtcagatcgcctggagacgccatccacgctgttttgacctccatagaagacaccggg | |
| | accgatccagcctcgagagacccaatgctagccaccatgaattgtagagaattacccttgaccctttgggtgcttatatc | |
| | tgtaagcactgcagaatcttgtacttcacgtccccacattactgtggttgaaggggaacctttctatctgaaacattgctcgt | |
| | gttcacttgcacatgagattgaaacaaccaccaaaagctggtacaaaagcagtggatcacaggaacatgtggagctga | |
| | acccaaggagttccagcagaattgctttgcatgattgtgttttggagttttggccagttgagttgaatgacacaggatctta | |
| | ctttttccaaatgaaaaattatactcagaaatggaaattaaatgtcatcagaagaaataaacacagctgtttcactgaaaga | |
| | caagtaactagtaaaattgtggaagttaaaaaattttttcagataacctgtgaaaacagttactatcaaacactggtcaaca | |
| | gcacatcattgtataagaactgtaaaaagctactactggagaacaataaaaacccaacgataaagaagaacgccgagt | |
| | ttgaagatcaggggtattactcctgcgtgcatttccttcatcataatggaaaactatttaatatcaccaaaaccttcaatataa | |
| | caatagtggaagatcgcagtaatatagttccggttcttcttggaccaaagcttaaccatgttgcagtggaattaggaaaaa | |
| | acgtaaggctcaactgctctgctttgctgaatgaagaggatgtaatttattggatgttcggggaagaaaatggatcggat | |
| | cctaatatacatgaagagaaagaaatgagaattatgactccagaaggcaaatggcatgcttcaaaagtattgagaattga | |
| | aaatattggtgaaagcaatctaaatgttttatataattgcactgtggccagcacgggaggcacagacaccaaaagcttca | |
| | tcttggtgagaaaagcagacatggctgatatcccaggccacgtcttcacaagaggaatgatcatagctgttttgatcttgg | |
| | tggcagtagtgtgcctagtgactgtgtgtgtcatttatagagttgacttggttctattttatagacatttaacgagaagagatg | |
| | aaacattaacagatggaaaaacatatgatgcttttgtgtcttacctaaaagaatgccgacctgaaaatggagaggagcac | |
| | acctttgctgtggagattttgcccagggtgttggagaaacattttgggtataagttatgcatatttgaaagggatgtagtgc | |
| | ctggaggagctgttgttgatgaaatccactcactgatagagaaaagccgaagactaatcattgtcctaagtaaaagttata | |
| | tgtctaatgaggtcaggtatgaacttgaaagtggactccatgaagcattggtggaaagaaaaattaaaataatcttaattg | |
| | aatttacacctgttactgacttcacattcttgccccaatcactaaagcttttgaaatctcacagagttctgaagtggaaggcc | |
| | gataaatctctttcttataactcaaggttctggaagaaccttctttacttaatgcctgcaaaaacagtcaagccaggtagag | |
| | acgaaccggaagtcttgcctgttctttccgagtctggaagcggtgagggaagaggtagcctgctcacgtgcggtgatg | |
| | tggaggagaaccctgggcctatgctctgtttgggctggatatttctttggcttgttgcaggagagcgaattaaaggattta | |
| | atatttcaggttgttccacaaaaaaactcctttggacatattctacaaggagtgaagaggaatttgtcttattttgtgatttacc | |
| | agagccacagaaatcacatttctgccacagaaatcgactctcaccaaaacaagtccctgagcacctgcccttcatgggt | |
| | agtaacgacctatctgatgtccaatggtaccaacaaccttcgaatggagatccattagaggacattaggaaaagctatcc | |
| | tcacatcattcaggacaaatgtacccttcactttttgaccccaggggtgaataattctgggtcatatatttgtagacccaag | |
| | atgattaagagcccctatgatgtagcctgttgtgtcaagatgattttagaagttaagccccagacaaatgcatcctgtgagt | |
| | attccgcatcacataagcaagacctacttcttgggagcactggctctatttcttgccccagtctcagctgccaaagtgatg | |
| | cacaaagtccagcggtaacctggtacaagaatggaaaactcctctctgtggaaaggagcaaccgaatcgtagtggatg | |
| | aagtttatgactatcaccagggcacatatgtatgtgattacactcagtcggatactgtgagttcgtggacagtcagagctg | |
| | ttgttcaagtgagaaccattgtgggagacactaaactcaaaccagatattctggatcctgtcgaggacacactggaagta | |
| | gaacttggaaagcctttaactattagctgcaaagcacgatttggctttgaaagggtctttaaccctgtcataaaatggtaca | |
| | tcaaagattctgacctagagtgggaagtctcagtacctgaggcgaaaagtattaaatccactttaaaggatgaaatcattg | |
| | agcgtaatatcatcttggaaaaagtcactcagcgtgatcttcgcaggaagtttgtttgctttgtccagaactccattggaaa | |
| | cacaacccagtccgtccaactgaaagaaaagagaggagtggtgctcctgtacatcctgcttggcaccatcgggaccct | |
| | ggtggccgtgctggcggcgagtgccctcctctacaggcactggattgaaatagtgctgctgtaccggacctaccagag | |
| | caaggatcagacgcttggggataaaaaggattttgatgctttcgtatcctatgcaaaatggagctcttttccaagtgaggc | |
| | cacttcatctctgagtgaagaacacttggccctgagcctatttcctgatgttttagaaaacaaatatggatatagcctgtgtt | |
| | tgcttgaaagagatgtggctccaggaggagtgtatgcagaagacattgtgagcattattaagagaagcagaagaggaa | |
| | tatttatcttgagccccaactatgtcaatggacccagtatctttgaactacaagcagcagtgaatcttgccttggatgatca | |
| | aacactgaaactcattttaattaagttctgttacttccaagagccagagtctctacctcatctcgtgaaaaaagctctcagg | |
| | gttttgcccacagttacttggagaggcttaaaatcagttcctcccaattctaggttctgggccaaaatgcgctaccacatg | |
| | cctgtgaaaaactctcagggattcacgtggaaccagctcagaattacctctaggatttttcagtggaaaggactcagtag | |
| | aacagaaaccactgggaggagctcccagcctaaggaatggtgataa | |
| CAR C2 CD28- | atggccttaccagtgaccgccttgctcctgccgctggccttgctgctccacgccgccaggccggaggtgcagctggtg | 142 |
| CD28-CD281XX | gagtctgggggaggcctggtcaagcctggggggtccctgagactctcctgtgcagcctctggattcaccttcagtggc | |
| 6XIL2NFAT | tatgccatgagctgggtccgccaggctccagggaaggggctggagtgggtctcaaccattagtagtggcggaaccta | |
| mCMV | catatactaccccgactcagtgaagggccgattcaccatctccagagacaacgccaagaactcactgtatctgcaaatg | |
| IL18Ralpha-T2A- | aacagcctgagagccgaggacacggccgtgtattactgtgcgagacttgggggggataattactacgaatacttcgat | |
| IL18Rb | gtctggggcaaagggaccacggtcaccgtctcctccggcggtggcggatccggcggtggcggatccggcggtggc | |
| | ggatccgacattgtgctgacccagtctccagcctccttggccgtgtctccaggacagagggccaccatcacctgcaga | |
| | gccagtaagagtgtcagtaccagcggatactcctacatgcactggtatcagcagaaaccaggacaacctcctaaactc | |
| | ctgatttacctggcatccaatctggagagcggggtcccagccaggttcagcggcagtgggtctgggaccgatttcacc | |
| | ctcacaattaatcctgtggaagctaatgatactgcaaattattactgtcagcacagtagggagctgcctttcacattcggc | |
| | ggagggaccaaggtggagatcaaacgaactaagcacctgtgtccttctccactgttccccggccctagcaagcctttct | |
| | gggtcctggtggtggtgggcggagtgctggcctgctacagcctgctggtgaccgtggcctttatcatcttctgggtgag | |
| | gagtaagaggagcaggctcctgcacagtgactacatgaacatgactcctagaagacctgggcctaccagaaagcatt | |
| | accagccctatgccccaccacgcgacttcgcagcctatcgctccagagtgaagttcagcaggagcgcagacgcccc | |
| | cgcgtacaagcagggccagaaccagctctataacgagctcaatctaggacgaagagaggagtacgatgttttggaca | |
| | agagacgtggccgggaccctgagatggggggaaagccgagaaggaagaaccctcaggaaggcctgttcaatgaa | |
| | ctgcagaaagataagatggcggaggccttcagtgagattgggatgaaaggcgagcgccggaggggcaaggggca | |
| | cgatggccttttccagggtctcagtacagccaccaaggacaccttcgacgcccttcacatgcaggccctgccccctcg | |
| | ctgataagtttaaactgccagaacattactctggcctaactggccggtaccggaggaaaaactgtttcatacagaaggc | |
| | gtggaggaaaaactgtttcatacagaaggcgtggaggaaaaactgtttcatacagaaggcgtggaggaaaaactgttt | |
| | catacagaaggcgtggaggaaaaactgtttcatacagaaggcgtggaggaaaaactgtttcatacagaaggcgtacta | |
| | gttaggcgtgtacggtgggaggcctatataagcagagctcgtttagtgaaccgtcagatcgcctggagacgccatcca | |
| | cgctgttttgacctccatagaagacaccgggaccgatccagcctcgagagacccaatgctagccaccatgaattgtag | |
| | agaattacccttgaccctttgggtgcttatatctgtaagcactgcagaatcttgtacttcacgtccccacattactgtggttg | |
| | aaggggaacctttctatctgaaacattgctcgtgttcacttgcacatgagattgaaacaaccaccaaaagctggtacaaa | |
| | agcagtggatcacaggaacatgtggagctgaacccaaggagttccagcagaattgctttgcatgattgtgttttggagtt | |
| | ttggccagttgagttgaatgacacaggatcttactttttccaaatgaaaaattatactcagaaatggaaattaaatgtcatca | |
| | gaagaaataaacacagctgtttcactgaaagacaagtaactagtaaaattgtggaagttaaaaaattttttcagataacct | |
| | gtgaaaacagttactatcaaacactggtcaacagcacatcattgtataagaactgtaaaaagctactactggagaacaat | |
| | aaaaacccaacgataaagaagaacgccgagtttgaagatcaggggtattactcctgcgtgcatttccttcatcataatgg | |
| | aaaactatttaatatcaccaaaaccttcaatataacaatagtggaagatcgcagtaatatagttccggttcttcttggaccaa | |
| | agcttaaccatgttgcagtggaattaggaaaaaacgtaaggctcaactgctctgctttgctgaatgaagaggatgtaattt | |
| | attggatgttcggggaagaaaatggatcggatcctaatatacatgaagagaaagaaatgagaattatgactccagaagg | |
| | caaatggcatgcttcaaaagtattgagaattgaaaatattggtgaaagcaatctaaatgttttatataattgcactgtggcca | |
| | gcacgggaggcacagacaccaaaagcttcatcttggtgagaaaagcagacatggctgatatcccaggccacgtcttc | |
| | acaagaggaatgatcatagctgttttgatcttggtggcagtagtgtgcctagtgactgtgtgtgtcatttatagagttgactt | |
| | ggttctattttatagacatttaacgagaagagatgaaacattaacagatggaaaaacatatgatgcttttgtgtcttacctaa | |
| | aagaatgccgacctgaaaatggagaggagcacacctttgctgtggagattttgcccagggtgttggagaaacattttgg | |
| | gtataagttatgcatatttgaaagggatgtagtgcctggaggagctgttgttgatgaaatccactcactgatagagaaaag | |
| | ccgaagactaatcattgtcctaagtaaaagttatatgtctaatgaggtcaggtatgaacttgaaagtggactccatgaagc | |
| | attggtggaaagaaaaattaaaataatcttaattgaatttacacctgttactgacttcacattcttgccccaatcactaaagct | |
| | tttgaaatctcacagagttctgaagtggaaggccgataaatctctttcttataactcaaggttctggaagaaccttctttactt | |
| | aatgcctgcaaaaacagtcaagccaggtagagacgaaccggaagtcttgcctgttctttccgagtctggaagcggtga | |
| | gggaagaggtagcctgctcacgtgcggtgatgtggaggagaaccctgggcctatgctctgtttgggctggatatttcttt | |
| | ggcttgttgcaggagagcgaattaaaggatttaatatttcaggttgttccacaaaaaaactcctttggacatattctacaag | |
| | gagtgaagaggaatttgtcttattttgtgatttaccagagccacagaaatcacatttctgccacagaaatcgactctcacca | |
| | aaacaagtccctgagcacctgcccttcatgggtagtaacgacctatctgatgtccaatggtaccaacaaccttcgaatgg | |
| | agatccattagaggacattaggaaaagctatcctcacatcattcaggacaaatgtacccttcactttttgaccccaggggt | |
| | gaataattctgggtcatatatttgtagacccaagatgattaagagcccctatgatgtagcctgttgtgtcaagatgattttag | |
| | aagttaagccccagacaaatgcatcctgtgagtattccgcatcacataagcaagacctacttcttgggagcactggctct | |
| | atttcttgccccagtctcagctgccaaagtgatgcacaaagtccagcggtaacctggtacaagaatggaaaactcctctc | |
| | tgtggaaaggagcaaccgaatcgtagtggatgaagtttatgactatcaccagggcacatatgtatgtgattacactcagt | |
| | cggatactgtgagttcgtggacagtcagagctgttgttcaagtgagaaccattgtgggagacactaaactcaaaccaga | |
| | tattctggatcctgtcgaggacacactggaagtagaacttggaaagcctttaactattagctgcaaagcacgatttggcttt | |
| | gaaagggtctttaaccctgtcataaaatggtacatcaaagattctgacctagagtgggaagtctcagtacctgaggcgaa | |
| | aagtattaaatccactttaaaggatgaaatcattgagcgtaatatcatcttggaaaaagtcactcagcgtgatcttcgcagg | |
| | aagtttgtttgctttgtccagaactccattggaaacacaacccagtccgtccaactgaaagaaaagagaggagtggtgct | |
| | cctgtacatcctgcttggcaccatcgggaccctggtggccgtgctggcggcgagtgccctcctctacaggcactggatt | |
| | gaaatagtgctgctgtaccggacctaccagagcaaggatcagacgcttggggataaaaaggattttgatgctttcgtatc | |
| | ctatgcaaaatggagctcttttccaagtgaggccacttcatctctgagtgaagaacacttggccctgagcctatttcctgat | |
| | gttttagaaaacaaatatggatatagcctgtgtttgcttgaaagagatgtggctccaggaggagtgtatgcagaagacatt | |
| | gtgagcattattaagagaagcagaagaggaatatttatcttgagccccaactatgtcaatggacccagtatctttgaacta | |
| | caagcagcagtgaatcttgccttggatgatcaaacactgaaactcattttaattaagttctgttacttccaagagccagagt | |
| | ctctacctcatctcgtgaaaaaagctctcagggttttgcccacagttacttggagaggcttaaaatcagttcctcccaattct | |
| | aggttctgggccaaaatgcgctaccacatgcctgtgaaaaactctcagggattcacgtggaaccagctcagaattacct | |
| | ctaggatttttcagtggaaaggactcagtagaacagaaaccactgggaggagctcccagcctaaggaatggtgataa | |
| CAR C2 CD28- | atggccttaccagtgaccgccttgctcctgccgctggccttgctgctccacgccgccaggccggaggtgcagctggtg | 143 |
| CD28-CD281XX | gagtctgggggaggcctggtcaagcctggggggtccctgagactctcctgtgcagcctctggattcaccttcagtggc | |
| 3XIL2NFAT | tatgccatgagctgggtccgccaggctccagggaaggggctggagtgggtctcaaccattagtagtggcggaaccta | |
| mCMV | catatactaccccgactcagtgaagggccgattcaccatctccagagacaacgccaagaactcactgtatctgcaaatg | |
| IL18Ralpha-T2A- | aacagcctgagagccgaggacacggccgtgtattactgtgcgagacttgggggggataattactacgaatacttcgat | |
| IL18Rb | gtctggggcaaagggaccacggtcaccgtctcctccggcggtggcggatccggcggtggcggatccggcggtggc | |
| | ggatccgacattgtgctgacccagtctccagcctccttggccgtgtctccaggacagagggccaccatcacctgcaga | |
| | gccagtaagagtgtcagtaccagcggatactcctacatgcactggtatcagcagaaaccaggacaacctcctaaactc | |
| | ctgatttacctggcatccaatctggagagcggggtcccagccaggttcagcggcagtgggtctgggaccgatttcacc | |
| | ctcacaattaatcctgtggaagctaatgatactgcaaattattactgtcagcacagtagggagctgcctttcacattcggc | |
| | ggagggaccaaggtggagatcaaacgaactaagcacctgtgtccttctccactgttccccggccctagcaagcctttct | |
| | gggtcctggtggtggtgggcggagtgctggcctgctacagcctgctggtgaccgtggcctttatcatcttctgggtgag | |
| | gagtaagaggagcaggctcctgcacagtgactacatgaacatgactcctagaagacctgggcctaccagaaagcatt | |
| | accagccctatgccccaccacgcgacttcgcagcctatcgctccagagtgaagttcagcaggagcgcagacgcccc | |
| | cgcgtacaagcagggccagaaccagctctataacgagctcaatctaggacgaagagaggagtacgatgttttggaca | |
| | agagacgtggccgggaccctgagatggggggaaagccgagaaggaagaaccctcaggaaggcctgttcaatgaa | |
| | ctgcagaaagataagatggcggaggccttcagtgagattgggatgaaaggcgagcgccggaggggcaaggggca | |
| | cgatggccttttccagggtctcagtacagccaccaaggacaccttcgacgcccttcacatgcaggccctgccccctcg | |
| | ctgataagtttaaactgccagaacattactctggcctaactggccggtaccggaggaaaaactgtttcatacagaaggc | |
| | gtggaggaaaaactgtttcatacagaaggcgtggaggaaaaactgtttcatacagaaggcgtactagttaggcgtgtac | |
| | ggtgggaggcctatataagcagagctcgtttagtgaaccgtcagatcgcctggagacgccatccacgctgttttgacct | |
| | ccatagaagacaccgggaccgatccagcctcgagagacccaatgctagccaccatgaattgtagagaattacccttg | |
| | accctttgggtgcttatatctgtaagcactgcagaatcttgtacttcacgtccccacattactgtggttgaaggggaaccttt | |
| | ctatctgaaacattgctcgtgttcacttgcacatgagattgaaacaaccaccaaaagctggtacaaaagcagtggatcac | |
| | aggaacatgtggagctgaacccaaggagttccagcagaattgctttgcatgattgtgttttggagttttggccagttgagt | |
| | tgaatgacacaggatcttactttttccaaatgaaaaattatactcagaaatggaaattaaatgtcatcagaagaaataaaca | |
| | cagctgtttcactgaaagacaagtaactagtaaaattgtggaagttaaaaaattttttcagataacctgtgaaaacagttact | |
| | atcaaacactggtcaacagcacatcattgtataagaactgtaaaaagctactactggagaacaataaaaacccaacgat | |
| | aaagaagaacgccgagtttgaagatcaggggtattactcctgcgtgcatttccttcatcataatggaaaactatttaatatc | |
| | accaaaaccttcaatataacaatagtggaagatcgcagtaatatagttccggttcttcttggaccaaagcttaaccatgttg | |
| | cagtggaattaggaaaaaacgtaaggctcaactgctctgctttgctgaatgaagaggatgtaatttattggatgttcggg | |
| | gaagaaaatggatcggatcctaatatacatgaagagaaagaaatgagaattatgactccagaaggcaaatggcatgctt | |
| | caaaagtattgagaattgaaaatattggtgaaagcaatctaaatgttttatataattgcactgtggccagcacgggaggca | |
| | cagacaccaaaagcttcatcttggtgagaaaagcagacatggctgatatcccaggccacgtcttcacaagaggaatga | |
| | tcatagctgttttgatcttggtggcagtagtgtgcctagtgactgtgtgtgtcatttatagagttgacttggttctattttataga | |
| | catttaacgagaagagatgaaacattaacagatggaaaaacatatgatgcttttgtgtcttacctaaaagaatgccgacct | |
| | gaaaatggagaggagcacacctttgctgtggagattttgcccagggtgttggagaaacattttgggtataagttatgcat | |
| | atttgaaagggatgtagtgcctggaggagctgttgttgatgaaatccactcactgatagagaaaagccgaagactaatc | |
| | attgtcctaagtaaaagttatatgtctaatgaggtcaggtatgaacttgaaagtggactccatgaagcattggtggaaaga | |
| | aaaattaaaataatcttaattgaatttacacctgttactgacttcacattcttgccccaatcactaaagcttttgaaatctcaca | |
| | gagttctgaagtggaaggccgataaatctctttcttataactcaaggttctggaagaaccttctttacttaatgcctgcaaaa | |
| | acagtcaagccaggtagagacgaaccggaagtcttgcctgttctttccgagtctggaagcggtgagggaagaggtag | |
| | cctgctcacgtgcggtgatgtggaggagaaccctgggcctatgctctgtttgggctggatatttctttggcttgttgcagg | |
| | agagcgaattaaaggatttaatatttcaggttgttccacaaaaaaactcctttggacatattctacaaggagtgaagagga | |
| | atttgtcttattttgtgatttaccagagccacagaaatcacatttctgccacagaaatcgactctcaccaaaacaagtccctg | |
| | agcacctgcccttcatgggtagtaacgacctatctgatgtccaatggtaccaacaaccttcgaatggagatccattagag | |
| | gacattaggaaaagctatcctcacatcattcaggacaaatgtacccttcactttttgaccccaggggtgaataattctgggt | |
| | catatatttgtagacccaagatgattaagagcccctatgatgtagcctgttgtgtcaagatgattttagaagttaagcccca | |
| | gacaaatgcatcctgtgagtattccgcatcacataagcaagacctacttcttgggagcactggctctatttcttgccccagt | |
| | ctcagctgccaaagtgatgcacaaagtccagcggtaacctggtacaagaatggaaaactcctctctgtggaaaggagc | |
| | aaccgaatcgtagtggatgaagtttatgactatcaccagggcacatatgtatgtgattacactcagtcggatactgtgagt | |
| | tcgtggacagtcagagctgttgttcaagtgagaaccattgtgggagacactaaactcaaaccagatattctggatcctgt | |
| | cgaggacacactggaagtagaacttggaaagcctttaactattagctgcaaagcacgatttggctttgaaagggtcttta | |
| | accctgtcataaaatggtacatcaaagattctgacctagagtgggaagtctcagtacctgaggcgaaaagtattaaatcc | |
| | actttaaaggatgaaatcattgagcgtaatatcatcttggaaaaagtcactcagcgtgatcttcgcaggaagtttgtttgctt | |
| | tgtccagaactccattggaaacacaacccagtccgtccaactgaaagaaaagagaggagtggtgctcctgtacatcct | |
| | gcttggcaccatcgggaccctggtggccgtgctggcggcgagtgccctcctctacaggcactggattgaaatagtgct | |
| | gctgtaccggacctaccagagcaaggatcagacgcttggggataaaaaggattttgatgctttcgtatcctatgcaaaat | |
| | ggagctcttttccaagtgaggccacttcatctctgagtgaagaacacttggccctgagcctatttcctgatgttttagaaaa | |
| | caaatatggatatagcctgtgtttgcttgaaagagatgtggctccaggaggagtgtatgcagaagacattgtgagcatta | |
| | ttaagagaagcagaagaggaatatttatcttgagccccaactatgtcaatggacccagtatctttgaactacaagcagca | |
| | gtgaatcttgccttggatgatcaaacactgaaactcattttaattaagttctgttacttccaagagccagagtctctacctcat | |
| | ctcgtgaaaaaagctctcagggttttgcccacagttacttggagaggcttaaaatcagttcctcccaattctaggttctggg | |
| | ccaaaatgcgctaccacatgcctgtgaaaaactctcagggattcacgtggaaccagctcagaattacctctaggatttttc | |
| | agtggaaaggactcagtagaacagaaaccactgggaggagctcccagcctaaggaatggtgataa | |
| CAR E6 CD8- | atggccttaccagtgaccgccttgctcctgccgctggccttgctgctccacgccgccaggccggaggtgcagctggtg | 144 |
| CD8-41BBCD3 | gagtctgggggaggcctggtcaagcctggggggtccctgagactctcctgtgcagcctctggattcaccttcagtagg | |
| 6XFOXP3NFAT | tatggcatgagctgggtccgccaggctccagggaagaggctggagtgggtctcaaccattagtggcggaggcacct | |
| mCMV | acatatactacccagactcagtgaagggccgattcaccatctccagagacaacgccaagaacaccctgtatctgcaaat | |
| IL18Ralpha-T2A- | gaacagcctgagagccgaggacacggctgtgtattactgtaccagagataactatggccgcaactatgattatggcatg | |
| IL18Rb | gattattggggccagggcaccctggtgaccgtgagcagcggtggtggcggttccggcggtggcggttccggtggtg | |
| | gcggttccgaaattgtgttgacacagtctccagccaccctgtctttgtctccaggggaaagagccaccctcacctgcag | |
| | cgccaccagcagtgttagctacatccactggtatcaacagaggcctggccagagccccaggctcctcatctatagcac | |
| | ctccaacctggccagcggcatcccagccaggttcagtggcagtgggtctgggagcgactacactctcaccatcagca | |
| | gcctagagcctgaagattttgcagtttattactgtcagcagcgtagcagctcccctttcacctttggcagcggcaccaaa | |
| | gtggaaattaaaacaacaacccctgcccccagacctcctaccccagcccctacaattgccagccagcctctgagcctg | |
| | aggcccgaggcttgtagacctgctgctggcggagccgtgcacaccagaggactggatttcgcctgcgacatctacatc | |
| | tgggcgcccttggccgggacttgtggggtccttctcctgtcactggttatcaccctttactgcaaacggggcagaaaga | |
| | aactcctgtatatattcaaacaaccatttatgagaccagtacaaactactcaagaggaagatggctgtagctgccgatttc | |
| | cagaagaagaagaaggaggatgtgaactgagagtgaagttcagcaggagcgcagacgcccccgcgtacaagcag | |
| | ggccagaaccagctctataacgagctcaatctaggacgaagagaggagtacgatgttttggacaagagacgtggccg | |
| | ggaccctgagatggggggaaagccgagaaggaagaaccctcaggaaggcctgtacaatgaactgcagaaagataa | |
| | gatggcggaggcctacagtgagattgggatgaaaggcgagcgccggaggggcaaggggcacgatggcctttacca | |
| | gggtctcagtacagccaccaaggacacctacgacgcccttcacatgcaggccctgccccctcgctgataagtttaaact | |
| | gccagaacatttctctggcctaactggccggtaccggcttcattttttccatttactgcagaggcttcattttttccatttactg | |
| | cagaggcttcattttttccatttactgcagaggcttcattttttccatttactgcagaggcttcattttttccatttactgcagag | |
| | gcttcattttttccatttactgcagaactagttaggcgtgtacggtgggaggcctatataagcagagctcgtttagtgaacc | |
| | gtcagatcgcctggagacgccatccacgctgttttgacctccatagaagacaccgggaccgatccagcctcgagaga | |
| | cccaatgctagccaccatgaattgtagagaattacccttgaccctttgggtgcttatatctgtaagcactgcagaatcttgt | |
| | acttcacgtccccacattactgtggttgaaggggaacctttctatctgaaacattgctcgtgttcacttgcacatgagattga | |
| | aacaaccaccaaaagctggtacaaaagcagtggatcacaggaacatgtggagctgaacccaaggagttccagcaga | |
| | attgctttgcatgattgtgttttggagttttggccagttgagttgaatgacacaggatcttactttttccaaatgaaaaattata | |
| | ctcagaaatggaaattaaatgtcatcagaagaaataaacacagctgtttcactgaaagacaagtaactagtaaaattgtg | |
| | gaagttaaaaaattttttcagataacctgtgaaaacagttactatcaaacactggtcaacagcacatcattgtataagaact | |
| | gtaaaaagctactactggagaacaataaaaacccaacgataaagaagaacgccgagtttgaagatcaggggtattact | |
| | cctgcgtgcatttccttcatcataatggaaaactatttaatatcaccaaaaccttcaatataacaatagtggaagatcgcagt | |
| | aatatagttccggttcttcttggaccaaagcttaaccatgttgcagtggaattaggaaaaaacgtaaggctcaactgctct | |
| | gctttgctgaatgaagaggatgtaatttattggatgttcggggaagaaaatggatcggatcctaatatacatgaagagaa | |
| | agaaatgagaattatgactccagaaggcaaatggcatgcttcaaaagtattgagaattgaaaatattggtgaaagcaatc | |
| | taaatgttttatataattgcactgtggccagcacgggaggcacagacaccaaaagcttcatcttggtgagaaaagcaga | |
| | catggctgatatcccaggccacgtcttcacaagaggaatgatcatagctgttttgatcttggtggcagtagtgtgcctagt | |
| | gactgtgtgtgtcatttatagagttgacttggttctattttatagacatttaacgagaagagatgaaacattaacagatggaa | |
| | aaacatatgatgcttttgtgtcttacctaaaagaatgccgacctgaaaatggagaggagcacacctttgctgtggagatttt | |
| | gcccagggtgttggagaaacattttgggtataagttatgcatatttgaaagggatgtagtgcctggaggagctgttgttga | |
| | tgaaatccactcactgatagagaaaagccgaagactaatcattgtcctaagtaaaagttatatgtctaatgaggtcaggta | |
| | tgaacttgaaagtggactccatgaagcattggtggaaagaaaaattaaaataatcttaattgaatttacacctgttactgac | |
| | ttcacattcttgccccaatcactaaagcttttgaaatctcacagagttctgaagtggaaggccgataaatctctttcttataac | |
| | tcaaggttctggaagaaccttctttacttaatgcctgcaaaaacagtcaagccaggtagagacgaaccggaagtcttgc | |
| | ctgttctttccgagtctggaagcggtgagggaagaggtagcctgctcacgtgcggtgatgtggaggagaaccctggg | |
| | cctatgctctgtttgggctggatatttctttggcttgttgcaggagagcgaattaaaggatttaatatttcaggttgttccaca | |
| | aaaaaactcctttggacatattctacaaggagtgaagaggaatttgtcttattttgtgatttaccagagccacagaaatcac | |
| | atttctgccacagaaatcgactctcaccaaaacaagtccctgagcacctgcccttcatgggtagtaacgacctatctgat | |
| | gtccaatggtaccaacaaccttcgaatggagatccattagaggacattaggaaaagctatcctcacatcattcaggaca | |
| | aatgtacccttcactttttgaccccaggggtgaataattctgggtcatatatttgtagacccaagatgattaagagcccctat | |
| | gatgtagcctgttgtgtcaagatgattttagaagttaagccccagacaaatgcatcctgtgagtattccgcatcacataag | |
| | caagacctacttcttgggagcactggctctatttcttgccccagtctcagctgccaaagtgatgcacaaagtccagcggt | |
| | aacctggtacaagaatggaaaactcctctctgtggaaaggagcaaccgaatcgtagtggatgaagtttatgactatcac | |
| | cagggcacatatgtatgtgattacactcagtcggatactgtgagttcgtggacagtcagagctgttgttcaagtgagaac | |
| | cattgtgggagacactaaactcaaaccagatattctggatcctgtcgaggacacactggaagtagaacttggaaagcct | |
| | ttaactattagctgcaaagcacgatttggctttgaaagggtctttaaccctgtcataaaatggtacatcaaagattctgacct | |
| | agagtgggaagtctcagtacctgaggcgaaaagtattaaatccactttaaaggatgaaatcattgagcgtaatatcatctt | |
| | ggaaaaagtcactcagcgtgatcttcgcaggaagtttgtttgctttgtccagaactccattggaaacacaacccagtccgt | |
| | ccaactgaaagaaaagagaggagtggtgctcctgtacatcctgcttggcaccatcgggaccctggtggccgtgctgg | |
| | cggcgagtgccctcctctacaggcactggattgaaatagtgctgctgtaccggacctaccagagcaaggatcagacg | |
| | cttggggataaaaaggattttgatgctttcgtatcctatgcaaaatggagctcttttccaagtgaggccacttcatctctgag | |
| | tgaagaacacttggccctgagcctatttcctgatgttttagaaaacaaatatggatatagcctgtgtttgcttgaaagagat | |
| | gtggctccaggaggagtgtatgcagaagacattgtgagcattattaagagaagcagaagaggaatatttatcttgagcc | |
| | ccaactatgtcaatggacccagtatctttgaactacaagcagcagtgaatcttgccttggatgatcaaacactgaaactca | |
| | ttttaattaagttctgttacttccaagagccagagtctctacctcatctcgtgaaaaaagctctcagggttttgcccacagtta | |
| | cttggagaggcttaaaatcagttcctcccaattctaggttctgggccaaaatgcgctaccacatgcctgtgaaaaactctc | |
| | agggattcacgtggaaccagctcagaattacctctaggatttttcagtggaaaggactcagtagaacagaaaccactgg | |
| | gaggagctcccagcctaaggaatggtgataa | |
| CAR E6 CD8- | atggccttaccagtgaccgccttgctcctgccgctggccttgctgctccacgccgccaggccggaggtgcagctggtg | 145 |
| CD8-41BBCD3 | gagtctgggggaggcctggtcaagcctggggggtccctgagactctcctgtgcagcctctggattcaccttcagtagg | |
| 3XFOXP3NFAT | tatggcatgagctgggtccgccaggctccagggaagaggctggagtgggtctcaaccattagtggcggaggcacct | |
| mCMV | acatatactacccagactcagtgaagggccgattcaccatctccagagacaacgccaagaacaccctgtatctgcaaat | |
| IL18Ralpha-T2A- | gaacagcctgagagccgaggacacggctgtgtattactgtaccagagataactatggccgcaactatgattatggcatg | |
| IL18Rb | gattattggggccagggcaccctggtgaccgtgagcagcggtggtggcggttccggcggtggcggttccggtggtg | |
| | gcggttccgaaattgtgttgacacagtctccagccaccctgtctttgtctccaggggaaagagccaccctcacctgcag | |
| | cgccaccagcagtgttagctacatccactggtatcaacagaggcctggccagagccccaggctctcatctatagcac | |
| | ctccaacctggccagcggcatcccagccaggttcagtggcagtgggtctgggagcgactacactctcaccatcagca | |
| | gcctagagcctgaagattttgcagtttattactgtcagcagcgtagcagctcccctttcacctttggcagcggcaccaaa | |
| | gtggaaattaaaacaacaacccctgcccccagacctcctaccccagcccctacaattgccagccagcctctgagcctg | |
| | aggcccgaggcttgtagacctgctgctggcggagccgtgcacaccagaggactggatttcgcctgcgacatctacatc | |
| | tgggcgcccttggccgggacttgtggggtccttctcctgtcactggttatcaccctttactgcaaacggggcagaaaga | |
| | aactcctgtatatattcaaacaaccatttatgagaccagtacaaactactcaagaggaagatggctgtagctgccgatttc | |
| | cagaagaagaagaaggaggatgtgaactgagagtgaagttcagcaggagcgcagacgcccccgcgtacaagcag | |
| | ggccagaaccagctctataacgagctcaatctaggacgaagagaggagtacgatgttttggacaagagacgtggccg | |
| | ggaccctgagatggggggaaagccgagaaggaagaaccctcaggaaggcctgtacaatgaactgcagaaagataa | |
| | gatggcggaggcctacagtgagattgggatgaaaggcgagcgccggaggggcaaggggcacgatggcctttacca | |
| | gggtctcagtacagccaccaaggacacctacgacgcccttcacatgcaggccctgccccctcgctgataagtttaaact | |
| | gccagaacatttctctggcctaactggccggtaccggcttcattttttccatttactgcagaggcttcattttttccatttactg | |
| | cagaggcttcattttttccatttactgcagaactagttaggcgtgtacggtgggaggcctatataagcagagctcgtttagt | |
| | gaaccgtcagatcgcctggagacgccatccacgctgttttgacctccatagaagacaccgggaccgatccagcctcg | |
| | agagacccaatgctagccaccatgaattgtagagaattacccttgaccctttgggtgcttatatctgtaagcactgcaga | |
| | atcttgtacttcacgtccccacattactgtggttgaaggggaacctttctatctgaaacattgctcgtgttcacttgcacatg | |
| | agattgaaacaaccaccaaaagctggtacaaaagcagtggatcacaggaacatgtggagctgaacccaaggagttcc | |
| | agcagaattgctttgcatgattgtgttttggagttttggccagttgagttgaatgacacaggatcttactttttccaaatgaaa | |
| | aattatactcagaaatggaaattaaatgtcatcagaagaaataaacacagctgtttcactgaaagacaagtaactagtaaa | |
| | attgtggaagttaaaaaattttttcagataacctgtgaaaacagttactatcaaacactggtcaacagcacatcattgtataa | |
| | gaactgtaaaaagctactactggagaacaataaaaacccaacgataaagaagaacgccgagtttgaagatcaggggt | |
| | attactcctgcgtgcatttccttcatcataatggaaaactatttaatatcaccaaaaccttcaatataacaatagtggaagatc | |
| | gcagtaatatagttccggttcttcttggaccaaagcttaaccatgttgcagtggaattaggaaaaaacgtaaggctcaact | |
| | gctctgctttgctgaatgaagaggatgtaatttattggatgttcggggaagaaaatggatcggatcctaatatacatgaag | |
| | agaaagaaatgagaattatgactccagaaggcaaatggcatgcttcaaaagtattgagaattgaaaatattggtgaaag | |
| | caatctaaatgttttatataattgcactgtggccagcacgggaggcacagacaccaaaagcttcatcttggtgagaaaag | |
| | cagacatggctgatatcccaggccacgtcttcacaagaggaatgatcatagctgttttgatcttggtggcagtagtgtgc | |
| | ctagtgactgtgtgtgtcatttatagagttgacttggttctattttatagacatttaacgagaagagatgaaacattaacagat | |
| | ggaaaaacatatgatgcttttgtgtcttacctaaaagaatgccgacctgaaaatggagaggagcacacctttgctgtgga | |
| | gattttgcccagggtgttggagaaacattttgggtataagttatgcatatttgaaagggatgtagtgcctggaggagctgtt | |
| | gttgatgaaatccactcactgatagagaaaagccgaagactaatcattgtcctaagtaaaagttatatgtctaatgaggtc | |
| | aggtatgaacttgaaagtggactccatgaagcattggtggaaagaaaaattaaaataatcttaattgaatttacacctgtta | |
| | ctgacttcacattcttgccccaatcactaaagcttttgaaatctcacagagttctgaagtggaaggccgataaatctctttct | |
| | tataactcaaggttctggaagaaccttctttacttaatgcctgcaaaaacagtcaagccaggtagagacgaaccggaagt | |
| | cttgcctgttctttccgagtctggaagcggtgagggaagaggtagcctgctcacgtgcggtgatgtggaggagaaccc | |
| | tgggcctatgctctgtttgggctggatatttctttggcttgttgcaggagagcgaattaaaggatttaatatttcaggttgttc | |
| | cacaaaaaaactcctttggacatattctacaaggagtgaagaggaatttgtcttattttgtgatttaccagagccacagaaa | |
| | tcacatttctgccacagaaatcgactctcaccaaaacaagtccctgagcacctgcccttcatgggtagtaacgacctatct | |
| | gatgtccaatggtaccaacaaccttcgaatggagatccattagaggacattaggaaaagctatcctcacatcattcagga | |
| | caaatgtacccttcactttttgaccccaggggtgaataattctgggtcatatatttgtagacccaagatgattaagagcccc | |
| | tatgatgtagcctgttgtgtcaagatgattttagaagttaagccccagacaaatgcatcctgtgagtattccgcatcacata | |
| | agcaagacctacttcttgggagcactggctctatttcttgccccagtctcagctgccaaagtgatgcacaaagtccagcg | |
| | gtaacctggtacaagaatggaaaactcctctctgtggaaaggagcaaccgaatcgtagtggatgaagtttatgactatca | |
| | ccagggcacatatgtatgtgattacactcagtcggatactgtgagttcgtggacagtcagagctgttgttcaagtgagaa | |
| | ccattgtgggagacactaaactcaaaccagatattctggatcctgtcgaggacacactggaagtagaacttggaaagcc | |
| | tttaactattagctgcaaagcacgatttggctttgaaagggtctttaaccctgtcataaaatggtacatcaaagattctgacc | |
| | tagagtgggaagtctcagtacctgaggcgaaaagtattaaatccactttaaaggatgaaatcattgagcgtaatatcatct | |
| | tggaaaaagtcactcagcgtgatcttcgcaggaagtttgtttgctttgtccagaactccattggaaacacaacccagtccg | |
| | tccaactgaaagaaaagagaggagtggtgctcctgtacatcctgcttggcaccatcgggaccctggtggccgtgctgg | |
| | cggcgagtgccctcctctacaggcactggattgaaatagtgctgctgtaccggacctaccagagcaaggatcagacg | |
| | cttggggataaaaaggattttgatgctttcgtatcctatgcaaaatggagctcttttccaagtgaggccacttcatctctgag | |
| | tgaagaacacttggccctgagcctatttcctgatgttttagaaaacaaatatggatatagcctgtgtttgcttgaaagagat | |
| | gtggctccaggaggagtgtatgcagaagacattgtgagcattattaagagaagcagaagaggaatatttatcttgagcc | |
| | ccaactatgtcaatggacccagtatctttgaactacaagcagcagtgaatcttgccttggatgatcaaacactgaaactca | |
| | ttttaattaagttctgttacttccaagagccagagtctctacctcatctcgtgaaaaaagctctcagggttttgcccacagtta | |
| | cttggagaggcttaaaatcagttcctcccaattctaggttctgggccaaaatgcgctaccacatgcctgtgaaaaactctc | |
| | agggattcacgtggaaccagctcagaattacctctaggatttttcagtggaaaggactcagtagaacagaaaccactgg | |
| | gaggagctcccagcctaaggaatggtgataa | |
| CAR E6 CD8- | atggccttaccagtgaccgccttgctcctgccgctggccttgctgctccacgccgccaggccggaggtgcagctggtg | 146 |
| CD8-41BBCD3 | gagtctgggggaggcctggtcaagcctggggggtccctgagactctcctgtgcagcctctggattcaccttcagtagg | |
| 6XIL2NFAT | tatggcatgagctgggtccgccaggctccagggaagaggctggagtgggtctcaaccattagtggcggaggcacct | |
| mCMV | acatatactacccagactcagtgaagggccgattcaccatctccagagacaacgccaagaacaccctgtatctgcaaat | |
| IL18Ralpha-T2A- | gaacagcctgagagccgaggacacggctgtgtattactgtaccagagataactatggccgcaactatgattatggcatg | |
| IL18Rb | gattattggggccagggcaccctggtgaccgtgagcagcggtggtggcggttccggcggtggcggttccggtggtg | |
| | gcggttccgaaattgtgttgacacagtctccagccaccctgtctttgtctccaggggaaagagccaccctcacctgcag | |
| | cgccaccagcagtgttagctacatccactggtatcaacagaggcctggccagagccccaggctcctcatctatagcac | |
| | ctccaacctggccagcggcatcccagccaggttcagtggcagtgggtctgggagcgactacactctcaccatcagca | |
| | gcctagagcctgaagattttgcagtttattactgtcagcagcgtagcagctcccctttcacctttggcagcggcaccaaa | |
| | gtggaaattaaaacaacaacccctgcccccagacctcctaccccagcccctacaattgccagccagcctctgagcctg | |
| | aggcccgaggcttgtagacctgctgctggcggagccgtgcacaccagaggactggatttcgcctgcgacatctacatc | |
| | tgggcgcccttggccgggacttgtggggtccttctcctgtcactggttatcaccctttactgcaaacggggcagaaaga | |
| | aactcctgtatatattcaaacaaccatttatgagaccagtacaaactactcaagaggaagatggctgtagctgccgatttc | |
| | cagaagaagaagaaggaggatgtgaactgagagtgaagttcagcaggagcgcagacgcccccgcgtacaagcag | |
| | ggccagaaccagctctataacgagctcaatctaggacgaagagaggagtacgatgttttggacaagagacgtggccg | |
| | ggaccctgagatggggggaaagccgagaaggaagaaccctcaggaaggcctgtacaatgaactgcagaaagataa | |
| | gatggcggaggcctacagtgagattgggatgaaaggcgagcgccggaggggcaaggggcacgatggcctttacca | |
| | gggtctcagtacagccaccaaggacacctacgacgcccttcacatgcaggccctgccccctcgctgataagtttaaact | |
| | gccagaacatttctctggcctaactggccggtaccggaggaaaaactgtttcatacagaaggcgtggaggaaaaactg | |
| | tttcatacagaaggcgtggaggaaaaactgtttcatacagaaggcgtggaggaaaaactgtttcatacagaaggcgtg | |
| | gaggaaaaactgtttcatacagaaggcgtggaggaaaaactgtttcatacagaaggcgtactagttaggcgtgtacggt | |
| | gggaggcctatataagcagagctcgtttagtgaaccgtcagatcgcctggagacgccatccacgctgttttgacctcca | |
| | tagaagacaccgggaccgatccagcctcgagagacccaatgctagccaccatgaattgtagagaattacccttgacc | |
| | ctttgggtgcttatatctgtaagcactgcagaatcttgtacttcacgtccccacattactgtggttgaaggggaacctttctat | |
| | ctgaaacattgctcgtgttcacttgcacatgagattgaaacaaccaccaaaagctggtacaaaagcagtggatcacagg | |
| | aacatgtggagctgaacccaaggagttccagcagaattgctttgcatgattgtgttttggagttttggccagttgagttgaa | |
| | tgacacaggatcttactttttccaaatgaaaaattatactcagaaatggaaattaaatgtcatcagaagaaataaacacag | |
| | ctgtttcactgaaagacaagtaactagtaaaattgtggaagttaaaaaattttttcagataacctgtgaaaacagttactatc | |
| | aaacactggtcaacagcacatcattgtataagaactgtaaaaagctactactggagaacaataaaaacccaacgataaa | |
| | gaagaacgccgagtttgaagatcaggggtattactcctgcgtgcatttccttcatcataatggaaaactatttaatatcacc | |
| | aaaaccttcaatataacaatagtggaagatcgcagtaatatagttccggttcttcttggaccaaagcttaaccatgttgcag | |
| | tggaattaggaaaaaacgtaaggctcaactgctctgctttgctgaatgaagaggatgtaatttattggatgttcggggaa | |
| | gaaaatggatcggatcctaatatacatgaagagaaagaaatgagaattatgactccagaaggcaaatggcatgcttcaa | |
| | aagtattgagaattgaaaatattggtgaaagcaatctaaatgttttatataattgcactgtggccagcacgggaggcacag | |
| | acaccaaaagcttcatcttggtgagaaaagcagacatggctgatatcccaggccacgtcttcacaagaggaatgatcat | |
| | agctgttttgatcttggtggcagtagtgtgcctagtgactgtgtgtgtcatttatagagttgacttggttctattttatagacatt | |
| | taacgagaagagatgaaacattaacagatggaaaaacatatgatgcttttgtgtcttacctaaaagaatgccgacctgaa | |
| | aatggagaggagcacacctttgctgtggagattttgcccagggtgttggagaaacattttgggtataagttatgcatattt | |
| | gaaagggatgtagtgcctggaggagctgttgttgatgaaatccactcactgatagagaaaagccgaagactaatcattg | |
| | tcctaagtaaaagttatatgtctaatgaggtcaggtatgaacttgaaagtggactccatgaagcattggtggaaagaaaa | |
| | attaaaataatcttaattgaatttacacctgttactgacttcacattcttgccccaatcactaaagcttttgaaatctcacagag | |
| | ttctgaagtggaaggccgataaatctctttcttataactcaaggttctggaagaaccttctttacttaatgcctgcaaaaaca | |
| | gtcaagccaggtagagacgaaccggaagtcttgcctgttctttccgagtctggaagcggtgagggaagaggtagcct | |
| | gctcacgtgcggtgatgtggaggagaaccctgggcctatgctctgtttgggctggatatttctttggcttgttgcaggaga | |
| | gcgaattaaaggatttaatatttcaggttgttccacaaaaaaactcctttggacatattctacaaggagtgaagaggaattt | |
| | gtcttattttgtgatttaccagagccacagaaatcacatttctgccacagaaatcgactctcaccaaaacaagtccctgag | |
| | cacctgcccttcatgggtagtaacgacctatctgatgtccaatggtaccaacaaccttcgaatggagatccattagagga | |
| | cattaggaaaagctatcctcacatcattcaggacaaatgtacccttcactttttgaccccaggggtgaataattctgggtca | |
| | tatatttgtagacccaagatgattaagagcccctatgatgtagcctgttgtgtcaagatgattttagaagttaagccccaga | |
| | caaatgcatcctgtgagtattccgcatcacataagcaagacctacttcttgggagcactggctctatttcttgccccagtct | |
| | cagctgccaaagtgatgcacaaagtccagcggtaacctggtacaagaatggaaaactcctctctgtggaaaggagca | |
| | accgaatcgtagtggatgaagtttatgactatcaccagggcacatatgtatgtgattacactcagtcggatactgtgagtt | |
| | cgtggacagtcagagctgttgttcaagtgagaaccattgtgggagacactaaactcaaaccagatattctggatcctgtc | |
| | gaggacacactggaagtagaacttggaaagcctttaactattagctgcaaagcacgatttggctttgaaagggtctttaa | |
| | ccctgtcataaaatggtacatcaaagattctgacctagagtgggaagtctcagtacctgaggcgaaaagtattaaatcca | |
| | ctttaaaggatgaaatcattgagcgtaatatcatcttggaaaaagtcactcagcgtgatcttcgcaggaagtttgtttgcttt | |
| | gtccagaactccattggaaacacaacccagtccgtccaactgaaagaaaagagaggagtggtgctcctgtacatcctg | |
| | cttggcaccatcgggaccctggtggccgtgctggcggcgagtgccctcctctacaggcactggattgaaatagtgctg | |
| | ctgtaccggacctaccagagcaaggatcagacgcttggggataaaaaggattttgatgctttcgtatcctatgcaaaatg | |
| | gagctcttttccaagtgaggccacttcatctctgagtgaagaacacttggccctgagcctatttcctgatgttttagaaaac | |
| | aaatatggatatagcctgtgtttgcttgaaagagatgtggctccaggaggagtgtatgcagaagacattgtgagcattatt | |
| | aagagaagcagaagaggaatatttatcttgagccccaactatgtcaatggacccagtatctttgaactacaagcagcagt | |
| | gaatcttgccttggatgatcaaacactgaaactcattttaattaagttctgttacttccaagagccagagtctctacctcatct | |
| | cgtgaaaaaagctctcagggttttgcccacagttacttggagaggcttaaaatcagttcctcccaattctaggttctgggc | |
| | caaaatgcgctaccacatgcctgtgaaaaactctcagggattcacgtggaaccagctcagaattacctctaggatttttca | |
| | gtggaaaggactcagtagaacagaaaccactgggaggagctcccagcctaaggaatggtgataa | |
| CAR E6 CD8- | atggccttaccagtgaccgccttgctcctgccgctggccttgctgctccacgccgccaggccggaggtgcagctggtg | 147 |
| CD8-41BBCD3 | gagtctgggggaggcctggtcaagcctggggggtccctgagactctcctgtgcagcctctggattcaccttcagtagg | |
| 3XIL2NFAT | tatggcatgagctgggtccgccaggctccagggaagaggctggagtgggtctcaaccattagtggcggaggcacct | |
| mCMV | acatatactacccagactcagtgaagggccgattcaccatctccagagacaacgccaagaacaccctgtatctgcaaat | |
| IL18Ralpha-T2A- | gaacagcctgagagccgaggacacggctgtgtattactgtaccagagataactatggccgcaactatgattatggcatg | |
| IL18Rb | gattattggggccagggcaccctggtgaccgtgagcagcggtggtggcggttccggcggtggcggttccggtggtg | |
| | gcggttccgaaattgtgttgacacagtctccagccaccctgtctttgtctccaggggaaagagccaccctcacctgcag | |
| | cgccaccagcagtgttagctacatccactggtatcaacagaggcctggccagagccccaggctcctcatctatagcac | |
| | ctccaacctggccagcggcatcccagccaggttcagtggcagtgggtctgggagcgactacactctcaccatcagca | |
| | gcctagagcctgaagattttgcagtttattactgtcagcagcgtagcagctcccctttcacctttggcagcggcaccaaa | |
| | gtggaaattaaaacaacaacccctgcccccagacctcctaccccagcccctacaattgccagccagcctctgagcctg | |
| | aggcccgaggcttgtagacctgctgctggcggagccgtgcacaccagaggactggatttcgcctgcgacatctacatc | |
| | tgggcgcccttggccgggacttgtggggtccttctcctgtcactggttatcaccctttactgcaaacggggcagaaaga | |
| | aactcctgtatatattcaaacaaccatttatgagaccagtacaaactactcaagaggaagatggctgtagctgccgatttc | |
| | cagaagaagaagaaggaggatgtgaactgagagtgaagttcagcaggagcgcagacgcccccgcgtacaagcag | |
| | ggccagaaccagctctataacgagctcaatctaggacgaagagaggagtacgatgttttggacaagagacgtggccg | |
| | ggaccctgagatggggggaaagccgagaaggaagaaccctcaggaaggcctgtacaatgaactgcagaaagataa | |
| | gatggcggaggcctacagtgagattgggatgaaaggcgagcgccggaggggcaaggggcacgatggcctttacca | |
| | gggtctcagtacagccaccaaggacacctacgacgcccttcacatgcaggccctgccccctcgctgataagtttaaact | |
| | gccagaacatttctctggcctaactggccggtaccggaggaaaaactgtttcatacagaaggcgtggaggaaaaactg | |
| | tttcatacagaaggcgtggaggaaaaactgtttcatacagaaggcgtactagttaggcgtgtacggtgggaggcctata | |
| | taagcagagctcgtttagtgaaccgtcagatcgcctggagacgccatccacgctgttttgacctccatagaagacaccg | |
| | ggaccgatccagcctcgagagacccaatgctagccaccatgaattgtagagaattacccttgaccctttgggtgcttat | |
| | atctgtaagcactgcagaatcttgtacttcacgtccccacattactgtggttgaaggggaacctttctatctgaaacattgct | |
| | cgtgttcacttgcacatgagattgaaacaaccaccaaaagctggtacaaaagcagtggatcacaggaacatgtggagc | |
| | tgaacccaaggagttccagcagaattgctttgcatgattgtgttttggagttttggccagttgagttgaatgacacaggatc | |
| | ttactttttccaaatgaaaaattatactcagaaatggaaattaaatgtcatcagaagaaataaacacagctgtttcactgaaa | |
| | gacaagtaactagtaaaattgtggaagttaaaaaattttttcagataacctgtgaaaacagttactatcaaacactggtcaa | |
| | cagcacatcattgtataagaactgtaaaaagctactactggagaacaataaaaacccaacgataaagaagaacgccga | |
| | gtttgaagatcaggggtattactcctgcgtgcatttccttcatcataatggaaaactatttaatatcaccaaaaccttcaatat | |
| | aacaatagtggaagatcgcagtaatatagttccggttcttcttggaccaaagcttaaccatgttgcagtggaattaggaaa | |
| | aaacgtaaggctcaactgctctgctttgctgaatgaagaggatgtaatttattggatgttcggggaagaaaatggatc gg | |
| | atcctaatatacatgaagagaaagaaatgagaattatgactccagaaggcaaatggcatgcttcaaaagtattgagaatt | |
| | gaaaatattggtgaaagcaatctaaatgttttatataattgcactgtggccagcacgggaggcacagacaccaaaagctt | |
| | catcttggtgagaaaagcagacatggctgatatcccaggccacgtcttcacaagaggaatgatcatagctgttttgatctt | |
| | ggtggcagtagtgtgcctagtgactgtgtgtgtcatttatagagttgacttggttctattttatagacatttaacgagaagag | |
| | atgaaacattaacagatggaaaaacatatgatgcttttgtgtcttacctaaaagaatgccgacctgaaaatggagaggag | |
| | cacacctttgctgtggagattttgcccagggtgttggagaaacattttgggtataagttatgcatatttgaaagggatgtag | |
| | tgcctggaggagctgttgttgatgaaatccactcactgatagagaaaagccgaagactaatcattgtcctaagtaaaagt | |
| | tatatgtctaatgaggtcaggtatgaacttgaaagtggactccatgaagcattggtggaaagaaaaattaaaataatctta | |
| | attgaatttacacctgttactgacttcacattcttgccccaatcactaaagcttttgaaatctcacagagttctgaagtggaa | |
| | ggccgataaatctctttcttataactcaaggttctggaagaaccttctttacttaatgcctgcaaaaacagtcaagccaggt | |
| | agagacgaaccggaagtcttgcctgttctttccgagtctggaagcggtgagggaagaggtagcctgctcacgtgcggt | |
| | gatgtggaggagaaccctgggcctatgctctgtttgggctggatatttctttggcttgttgcaggagagcgaattaaagg | |
| | atttaatatttcaggttgttccacaaaaaaactcctttggacatattctacaaggagtgaagaggaatttgtcttattttgtgat | |
| | ttaccagagccacagaaatcacatttctgccacagaaatcgactctcaccaaaacaagtccctgagcacctgcccttcat | |
| | gggtagtaacgacctatctgatgtccaatggtaccaacaaccttcgaatggagatccattagaggacattaggaaaagc | |
| | tatcctcacatcattcaggacaaatgtacccttcactttttgaccccaggggtgaataattctgggtcatatatttgtagacc | |
| | caagatgattaagagcccctatgatgtagcctgttgtgtcaagatgattttagaagttaagccccagacaaatgcatcctg | |
| | tgagtattccgcatcacataagcaagacctacttcttgggagcactggctctatttcttgccccagtctcagctgccaaagt | |
| | gatgcacaaagtccagcggtaacctggtacaagaatggaaaactcctctctgtggaaaggagcaaccgaatcgtagtg | |
| | gatgaagtttatgactatcaccagggcacatatgtatgtgattacactcagtcggatactgtgagttcgtggacagtcaga | |
| | gctgttgttcaagtgagaaccattgtgggagacactaaactcaaaccagatattctggatcctgtcgaggacacactgga | |
| | agtagaacttggaaagcctttaactattagctgcaaagcacgatttggctttgaaagggtctttaaccctgtcataaaatgg | |
| | tacatcaaagattctgacctagagtgggaagtctcagtacctgaggcgaaaagtattaaatccactttaaaggatgaaat | |
| | cattgagcgtaatatcatcttggaaaaagtcactcagcgtgatcttcgcaggaagtttgtttgctttgtccagaactccattg | |
| | gaaacacaacccagtccgtccaactgaaagaaaagagaggagtggtgctcctgtacatcctgcttggcaccatcggg | |
| | accctggtggccgtgctggcggcgagtgccctcctctacaggcactggattgaaatagtgctgctgtaccggacctac | |
| | cagagcaaggatcagacgcttggggataaaaaggattttgatgctttcgtatcctatgcaaaatggagctcttttccaagt | |
| | gaggccacttcatctctgagtgaagaacacttggccctgagcctatttcctgatgttttagaaaacaaatatggatatagc | |
| | ctgtgtttgcttgaaagagatgtggctccaggaggagtgtatgcagaagacattgtgagcattattaagagaagcagaa | |
| | gaggaatatttatcttgagccccaactatgtcaatggacccagtatctttgaactacaagcagcagtgaatcttgccttgg | |
| | atgatcaaacactgaaactcattttaattaagttctgttacttccaagagccagagtctctacctcatctcgtgaaaaaagct | |
| | ctcagggttttgcccacagttacttggagaggcttaaaatcagttcctcccaattctaggttctgggccaaaatgcgctac | |
| | cacatgcctgtgaaaaactctcagggattcacgtggaaccagctcagaattacctctaggatttttcagtggaaaggact | |
| | cagtagaacagaaaccactgggaggagctcccagcctaaggaatggtgataa | |
| CAR E6 CD28- | atggccttaccagtgaccgccttgctcctgccgctggccttgctgctccacgccgccaggccggaggtgcagctggtg | 148 |
| CD28-CD28CD3 | gagtctgggggaggcctggtcaagcctggggggtccctgagactctcctgtgcagcctctggattcaccttcagtagg | |
| 6XFOXP3NFAT | tatggcatgagctgggtccgccaggctccagggaagaggctggagtgggtctcaaccattagtggcggaggcacct | |
| mCMV | acatatactacccagactcagtgaagggccgattcaccatctccagagacaacgccaagaacaccctgtatctgcaaat | |
| IL18Ralpha-T2A- | gaacagcctgagagccgaggacacggctgtgtattactgtaccagagataactatggccgcaactatgattatggcatg | |
| IL18Rb | gattattggggccagggcaccctggtgaccgtgagcagcggtggtggcggttccggcggtggcggttccggtggtg | |
| | gcggttccgaaattgtgttgacacagtctccagccaccctgtctttgtctccaggggaaagagccaccctcacctgcag | |
| | cgccaccagcagtgttagctacatccactggtatcaacagaggcctggccagagccccaggctcctcatctatagcac | |
| | ctccaacctggccagcggcatcccagccaggttcagtggcagtgggtctgggagcgactacactctcaccatcagca | |
| | gcctagagcctgaagattttgcagtttattactgtcagcagcgtagcagctcccctttcacctttggcagcggcaccaaa | |
| | gtggaaattaaaaagcacctgtgtccttctccactgttccccggccctagcaagcctttctgggtcctggtggtggtggg | |
| | cggagtgctggcctgctacagcctgctggtgaccgtggcctttatcatcttctgggtgaggagtaagaggagcaggct | |
| | cctgcacagtgactacatgaacatgactcctagaagacctgggcctaccagaaagcattaccagccctatgccccacc | |
| | acgcgacttcgcagcctatcgctccagagtgaagttcagcaggagcgcagacgcccccgcgtacaagcagggcca | |
| | gaaccagctctataacgagctcaatctaggacgaagagaggagtacgatgttttggacaagagacgtggccgggacc | |
| | ctgagatggggggaaagccgagaaggaagaaccctcaggaaggcctgtacaatgaactgcagaaagataagatgg | |
| | cggaggcctacagtgagattgggatgaaaggcgagcgccggaggggcaaggggcacgatggcctttaccagggtc | |
| | tcagtacagccaccaaggacacctacgacgcccttcacatgcaggccctgccccctcgctgataagtttaaactgcca | |
| | gaacatttctctggcctaactggccggtaccggcttcattttttccatttactgcagaggcttcattttttccatttactgcaga | |
| | ggcttcattttttccatttactgcagaggcttcattttttccatttactgcagaggcttcattttttccatttactgcagaggcttc | |
| | attttttccatttactgcagaactagttaggcgtgtacggtgggaggcctatataagcagagctcgtttagtgaaccgtca | |
| | gatcgcctggagacgccatccacgctgttttgacctccatagaagacaccgggaccgatccagcctcgagagaccca | |
| | atgctagccaccatgaattgtagagaattacccttgaccctttgggtgcttatatctgtaagcactgcagaatcttgtactt | |
| | cacgtccccacattactgtggttgaaggggaacctttctatctgaaacattgctcgtgttcacttgcacatgagattgaaac | |
| | aaccaccaaaagctggtacaaaagcagtggatcacaggaacatgtggagctgaacccaaggagttccagcagaattg | |
| | ctttgcatgattgtgttttggagttttggccagttgagttgaatgacacaggatcttactttttccaaatgaaaaattatactca | |
| | gaaatggaaattaaatgtcatcagaagaaataaacacagctgtttcactgaaagacaagtaactagtaaaattgtggaag | |
| | ttaaaaaattttttcagataacctgtgaaaacagttactatcaaacactggtcaacagcacatcattgtataagaactgtaaa | |
| | aagctactactggagaacaataaaaacccaacgataaagaagaacgccgagtttgaagatcaggggtattactcctgc | |
| | gtgcatttccttcatcataatggaaaactatttaatatcaccaaaaccttcaatataacaatagtggaagatcgcagtaatat | |
| | agttccggttcttcttggaccaaagcttaaccatgttgcagtggaattaggaaaaaacgtaaggctcaactgctctgctttg | |
| | ctgaatgaagaggatgtaatttattggatgttcggggaagaaaatggatcggatcctaatatacatgaagagaaagaaat | |
| | gagaattatgactccagaaggcaaatggcatgcttcaaaagtattgagaattgaaaatattggtgaaagcaatctaaatgt | |
| | tttatataattgcactgtggccagcacgggaggcacagacaccaaaagcttcatcttggtgagaaaagcagacatggct | |
| | gatatcccaggccacgtcttcacaagaggaatgatcatagctgttttgatcttggtggcagtagtgtgcctagtgactgtg | |
| | tgtgtcatttatagagttgacttggttctattttatagacatttaacgagaagagatgaaacattaacagatggaaaaacata | |
| | tgatgcttttgtgtcttacctaaaagaatgccgacctgaaaatggagaggagcacacctttgctgtggagattttgcccag | |
| | ggtgttggagaaacattttgggtataagttatgcatatttgaaagggatgtagtgcctggaggagctgttgttgatgaaatc | |
| | cactcactgatagagaaaagccgaagactaatcattgtcctaagtaaaagttatatgtctaatgaggtcaggtatgaactt | |
| | gaaagtggactccatgaagcattggtggaaagaaaaattaaaataatcttaattgaatttacacctgttactgacttcacatt | |
| | cttgccccaatcactaaagcttttgaaatctcacagagttctgaagtggaaggccgataaatctctttcttataactcaaggt | |
| | tctggaagaaccttctttacttaatgcctgcaaaaacagtcaagccaggtagagacgaaccggaagtcttgcctgttcttt | |
| | ccgagtctggaagcggtgagggaagaggtagcctgctcacgtgcggtgatgtggaggagaaccctgggcctatgct | |
| | ctgtttgggctggatatttctttggcttgttgcaggagagcgaattaaaggatttaatatttcaggttgttccacaaaaaaact | |
| | cctttggacatattctacaaggagtgaagaggaatttgtcttattttgtgatttaccagagccacagaaatcacatttctgcc | |
| | acagaaatcgactctcaccaaaacaagtccctgagcacctgcccttcatgggtagtaacgacctatctgatgtccaatg | |
| | gtaccaacaaccttcgaatggagatccattagaggacattaggaaaagctatcctcacatcattcaggacaaatgtaccc | |
| | ttcactttttgaccccaggggtgaataattctgggtcatatatttgtagacccaagatgattaagagcccctatgatgtagc | |
| | ctgttgtgtcaagatgattttagaagttaagccccagacaaatgcatcctgtgagtattccgcatcacataagcaagacct | |
| | acttcttgggagcactggctctatttcttgccccagtctcagctgccaaagtgatgcacaaagtccagcggtaacctggt | |
| | acaagaatggaaaactcctctctgtggaaaggagcaaccgaatcgtagtggatgaagtttatgactatcaccagggca | |
| | catatgtatgtgattacactcagtcggatactgtgagttcgtggacagtcagagctgttgttcaagtgagaaccattgtgg | |
| | gagacactaaactcaaaccagatattctggatcctgtcgaggacacactggaagtagaacttggaaagcctttaactatt | |
| | agctgcaaagcacgatttggctttgaaagggtctttaaccctgtcataaaatggtacatcaaagattctgacctagagtgg | |
| | gaagtctcagtacctgaggcgaaaagtattaaatccactttaaaggatgaaatcattgagcgtaatatcatcttggaaaaa | |
| | gtcactcagcgtgatcttcgcaggaagtttgtttgctttgtccagaactccattggaaacacaacccagtccgtccaactg | |
| | aaagaaaagagaggagtggtgctcctgtacatcctgcttggcaccatcgggaccctggtggccgtgctggcggcgag | |
| | tgccctcctctacaggcactggattgaaatagtgctgctgtaccggacctaccagagcaaggatcagacgcttggggat | |
| | aaaaaggattttgatgctttcgtatcctatgcaaaatggagctcttttccaagtgaggccacttcatctctgagtgaagaac | |
| | acttggccctgagcctatttcctgatgttttagaaaacaaatatggatatagcctgtgtttgcttgaaagagatgtggctcca | |
| | ggaggagtgtatgcagaagacattgtgagcattattaagagaagcagaagaggaatatttatcttgagccccaactatgt | |
| | caatggacccagtatctttgaactacaagcagcagtgaatcttgccttggatgatcaaacactgaaactcattttaattaag | |
| | ttctgttacttccaagagccagagtctctacctcatctcgtgaaaaaagctctcagggttttgcccacagttacttggagag | |
| | gcttaaaatcagttcctcccaattctaggttctgggccaaaatgcgctaccacatgcctgtgaaaaactctcagggattca | |
| | cgtggaaccagctcagaattacctctaggatttttcagtggaaaggactcagtagaacagaaaccactgggaggagct | |
| | cccagcctaaggaatggtgataa | |
| CAR E6 CD28- | atggccttaccagtgaccgccttgctcctgccgctggccttgctgctccacgccgccaggccggaggtgcagctggtg | 149 |
| CD28-CD28CD3 | gagtctgggggaggcctggtcaagcctggggggtccctgagactctcctgtgcagcctctggattcaccttcagtagg | |
| 3XFOXP3NFAT | tatggcatgagctgggtccgccaggctccagggaagaggctggagtgggtctcaaccattagtggggaggcacct | |
| mCMV | acatatactacccagactcagtgaagggccgattcaccatctccagagacaacgccaagaacaccctgtatctgcaaat | |
| IL18Ralpha-T2A- | gaacagcctgagagccgaggacacggctgtgtattactgtaccagagataactatggccgcaactatgattatggcatg | |
| IL18Rb | gattattggggccagggcaccctggtgaccgtgagcagcggtggtggcggttccggcggtggcggttccggtggtg | |
| | gcggttccgaaattgtgttgacacagtctccagccaccctgtctttgtctccaggggaaagagccaccctcacctgcag | |
| | cgccaccagcagtgttagctacatccactggtatcaacagaggcctggccagagccccaggctcctcatctatagcac | |
| | ctccaacctggccagcggcatcccagccaggttcagtggcagtgggtctgggagcgactacactctcaccatcagca | |
| | gcctagagcctgaagattttgcagtttattactgtcagcagcgtagcagctcccctttcacctttggcagcggcaccaaa | |
| | gtggaaattaaaaagcacctgtgtccttctccactgttccccggccctagcaagcctttctgggtcctggtggtggtggg | |
| | cggagtgctggcctgctacagcctgctggtgaccgtggcctttatcatcttctgggtgaggagtaagaggagcaggct | |
| | cctgcacagtgactacatgaacatgactcctagaagacctgggcctaccagaaagcattaccagccctatgccccacc | |
| | acgcgacttcgcagcctatcgctccagagtgaagttcagcaggagcgcagacgcccccgcgtacaagcagggcca | |
| | gaaccagctctataacgagctcaatctaggacgaagagaggagtacgatgttttggacaagagacgtggccgggacc | |
| | ctgagatggggggaaagccgagaaggaagaaccctcaggaaggcctgtacaatgaactgcagaaagataagatgg | |
| | cggaggcctacagtgagattgggatgaaaggcgagcgccggaggggcaaggggcacgatggcctttaccagggtc | |
| | tcagtacagccaccaaggacacctacgacgcccttcacatgcaggccctgccccctcgctgataagtttaaactgcca | |
| | gaacatttctctggcctaactggccggtaccggcttcattttttccatttactgcagaggcttcattttttccatttactgcaga | |
| | ggcttcattttttccatttactgcagaactagttaggcgtgtacggtgggaggcctatataagcagagctcgtttagtgaac | |
| | cgtcagatcgcctggagacgccatccacgctgttttgacctccatagaagacaccgggaccgatccagcctcgagag | |
| | acccaatgctagccaccatgaattgtagagaattacccttgaccctttgggtgcttatatctgtaagcactgcagaatctt | |
| | gtacttcacgtccccacattactgtggttgaaggggaacctttctatctgaaacattgctcgtgttcacttgcacatgagatt | |
| | gaaacaaccaccaaaagctggtacaaaagcagtggatcacaggaacatgtggagctgaacccaaggagttccagca | |
| | gaattgctttgcatgattgtgttttggagttttggccagttgagttgaatgacacaggatcttactttttccaaatgaaaaatta | |
| | tactcagaaatggaaattaaatgtcatcagaagaaataaacacagctgtttcactgaaagacaagtaactagtaaaattgt | |
| | ggaagttaaaaaattttttcagataacctgtgaaaacagttactatcaaacactggtcaacagcacatcattgtataagaac | |
| | tgtaaaaagctactactggagaacaataaaaacccaacgataaagaagaacgccgagtttgaagatcaggggtattac | |
| | tcctgcgtgcatttccttcatcataatggaaaactatttaatatcaccaaaaccttcaatataacaatagtggaagatcgcag | |
| | taatatagttccggttcttcttggaccaaagcttaaccatgttgcagtggaattaggaaaaaacgtaaggctcaactgctct | |
| | gctttgctgaatgaagaggatgtaatttattggatgttcggggaagaaaatggatcggatcctaatatacatgaagagaa | |
| | agaaatgagaattatgactccagaaggcaaatggcatgcttcaaaagtattgagaattgaaaatattggtgaaagcaatc | |
| | taaatgttttatataattgcactgtggccagcacgggaggcacagacaccaaaagcttcatcttggtgagaaaagcaga | |
| | catggctgatatcccaggccacgtcttcacaagaggaatgatcatagctgttttgatcttggtggcagtagtgtgcctagt | |
| | gactgtgtgtgtcatttatagagttgacttggttctattttatagacatttaacgagaagagatgaaacattaacagatggaa | |
| | aaacatatgatgcttttgtgtcttacctaaaagaatgccgacctgaaaatggagaggagcacacctttgctgtggagatttt | |
| | gcccagggtgttggagaaacattttgggtataagttatgcatatttgaaagggatgtagtgcctggaggagctgttgttga | |
| | tgaaatccactcactgatagagaaaagccgaagactaatcattgtcctaagtaaaagttatatgtctaatgaggtcaggta | |
| | tgaacttgaaagtggactccatgaagcattggtggaaagaaaaattaaaataatcttaattgaatttacacctgttactgac | |
| | ttcacattcttgccccaatcactaaagcttttgaaatctcacagagttctgaagtggaaggccgataaatctctttcttataac | |
| | tcaaggttctggaagaaccttctttacttaatgcctgcaaaaacagtcaagccaggtagagacgaaccggaagtcttgc | |
| | ctgttctttccgagtctggaagcggtgagggaagaggtagcctgctcacgtgcggtgatgtggaggagaaccctggg | |
| | cctatgctctgtttgggctggatatttctttggcttgttgcaggagagcgaattaaaggatttaatatttcaggttgttccaca | |
| | aaaaaactcctttggacatattctacaaggagtgaagaggaatttgtcttattttgtgatttaccagagccacagaaatcac | |
| | atttctgccacagaaatcgactctcaccaaaacaagtccctgagcacctgcccttcatgggtagtaacgacctatctgat | |
| | gtccaatggtaccaacaaccttcgaatggagatccattagaggacattaggaaaagctatcctcacatcattcaggaca | |
| | aatgtacccttcactttttgaccccaggggtgaataattctgggtcatatatttgtagacccaagatgattaagagcccctat | |
| | gatgtagcctgttgtgtcaagatgattttagaagttaagccccagacaaatgcatcctgtgagtattccgcatcacataag | |
| | caagacctacttcttgggagcactggctctatttcttgccccagtctcagctgccaaagtgatgcacaaagtccagcggt | |
| | aacctggtacaagaatggaaaactcctctctgtggaaaggagcaaccgaatcgtagtggatgaagtttatgactatcac | |
| | cagggcacatatgtatgtgattacactcagtcggatactgtgagttcgtggacagtcagagctgttgttcaagtgagaac | |
| | cattgtgggagacactaaactcaaaccagatattctggatcctgtcgaggacacactggaagtagaacttggaaagcct | |
| | ttaactattagctgcaaagcacgatttggctttgaaagggtctttaaccctgtcataaaatggtacatcaaagattctgacct | |
| | agagtgggaagtctcagtacctgaggcgaaaagtattaaatccactttaaaggatgaaatcattgagcgtaatatcatctt | |
| | ggaaaaagtcactcagcgtgatcttcgcaggaagtttgtttgctttgtccagaactccattggaaacacaacccagtccgt | |
| | ccaactgaaagaaaagagaggagtggtgctcctgtacatcctgcttggcaccatcgggaccctggtggccgtgctgg | |
| | cggcgagtgccctcctctacaggcactggattgaaatagtgctgctgtaccggacctaccagagcaaggatcagacg | |
| | cttggggataaaaaggattttgatgctttcgtatcctatgcaaaatggagctcttttccaagtgaggccacttcatctctgag | |
| | tgaagaacacttggccctgagcctatttcctgatgttttagaaaacaaatatggatatagcctgtgtttgcttgaaagagat | |
| | gtggctccaggaggagtgtatgcagaagacattgtgagcattattaagagaagcagaagaggaatatttatcttgagcc | |
| | ccaactatgtcaatggacccagtatctttgaactacaagcagcagtgaatcttgccttggatgatcaaacactgaaactca | |
| | ttttaattaagttctgttacttccaagagccagagtctctacctcatctcgtgaaaaaagctctcagggttttgcccacagtta | |
| | cttggagaggcttaaaatcagttcctcccaattctaggttctgggccaaaatgcgctaccacatgcctgtgaaaaactctc | |
| | agggattcacgtggaaccagctcagaattacctctaggatttttcagtggaaaggactcagtagaacagaaaccactgg | |
| | gaggagctcccagcctaaggaatggtgataa | |
| CAR E6 CD28- | atggccttaccagtgaccgccttgctcctgccgctggccttgctgctccacgccgccaggccggaggtgcagctggtg | 150 |
| CD28-CD28 CD3 | gagtctgggggaggcctggtcaagcctggggggtccctgagactctcctgtgcagcctctggattcaccttcagtagg | |
| 6XIL2NFAT | tatggcatgagctgggtccgccaggctccagggaagaggctggagtgggtctcaaccattagtggcggaggcacct | |
| mCMV | acatatactacccagactcagtgaagggccgattcaccatctccagagacaacgccaagaacaccctgtatctgcaaat | |
| IL18Ralpha-T2A- | gaacagcctgagagccgaggacacggctgtgtattactgtaccagagataactatggccgcaactatgattatggcatg | |
| IL18Rb | gattattggggccagggcaccctggtgaccgtgagcagcggtggtggcggttccggcggtggcggttccggtggtg | |
| | gcggttccgaaattgtgttgacacagtctccagccaccctgtctttgtctccaggggaaagagccaccctcacctgcag | |
| | cgccaccagcagtgttagctacatccactggtatcaacagaggcctggccagagccccaggctcctcatctatagcac | |
| | ctccaacctggccagcggcatcccagccaggttcagtggcagtgggtctgggagcgactacactctcaccatcagca | |
| | gcctagagcctgaagattttgcagtttattactgtcagcagcgtagcagctcccctttcacctttggcagcggcaccaaa | |
| | gtggaaattaaaaagcacctgtgtccttctccactgttccccggccctagcaagcctttctgggtcctggtggtggggg | |
| | cggagtgctggcctgctacagcctgctggtgaccgtggcctttatcatcttctgggtgaggagtaagaggagcaggct | |
| | cctgcacagtgactacatgaacatgactcctagaagacctgggcctaccagaaagcattaccagccctatgccccacc | |
| | acgcgacttcgcagcctatcgctccagagtgaagttcagcaggagcgcagacgcccccgcgtacaagcagggcca | |
| | gaaccagctctataacgagctcaatctaggacgaagagaggagtacgatgttttggacaagagacgtggccgggacc | |
| | ctgagatggggggaaagccgagaaggaagaaccctcaggaaggcctgtacaatgaactgcagaaagataagatgg | |
| | cggaggcctacagtgagattgggatgaaaggcgagcgccggaggggcaaggggcacgatggcctttaccagggtc | |
| | tcagtacagccaccaaggacacctacgacgcccttcacatgcaggccctgccccctcgctgataagtttaaactgcca | |
| | gaacatttctctggcctaactggccggtaccggaggaaaaactgtttcatacagaaggcgtggaggaaaaactgtttca | |
| | tacagaaggcgtggaggaaaaactgtttcatacagaaggcgtggaggaaaaactgtttcatacagaaggcgtggagg | |
| | aaaaactgtttcatacagaaggcgtggaggaaaaactgtttcatacagaaggcgtactagttaggcgtgtacggtggga | |
| | ggcctatataagcagagctcgtttagtgaaccgtcagatcgcctggagacgccatccacgctgttttgacctccatagaa | |
| | gacaccgggaccgatccagcctcgagagacccaatgctagccaccatgaattgtagagaattacccttgaccctttgg | |
| | gtgcttatatctgtaagcactgcagaatcttgtacttcacgtccccacattactgtggttgaaggggaacctttctatctgaa | |
| | acattgctcgtgttcacttgcacatgagattgaaacaaccaccaaaagctggtacaaaagcagtggatcacaggaacat | |
| | gtggagctgaacccaaggagttccagcagaattgctttgcatgattgtgttttggagttttggccagttgagttgaatgac | |
| | acaggatcttactttttccaaatgaaaaattatactcagaaatggaaattaaatgtcatcagaagaaataaacacagctgttt | |
| | cactgaaagacaagtaactagtaaaattgtggaagttaaaaaattttttcagataacctgtgaaaacagttactatcaaaca | |
| | ctggtcaacagcacatcattgtataagaactgtaaaaagctactactggagaacaataaaaacccaacgataaagaaga | |
| | acgccgagtttgaagatcaggggtattactcctgcgtgcatttccttcatcataatggaaaactatttaatatcaccaaaac | |
| | cttcaatataacaatagtggaagatcgcagtaatatagttccggttcttcttggaccaaagcttaaccatgttgcagtggaa | |
| | ttaggaaaaaacgtaaggctcaactgctctgctttgctgaatgaagaggatgtaatttattggatgttcggggaagaaaat | |
| | ggatcggatcctaatatacatgaagagaaagaaatgagaattatgactccagaaggcaaatggcatgcttcaaaagtat | |
| | tgagaattgaaaatattggtgaaagcaatctaaatgttttatataattgcactgtggccagcacgggaggcacagacacc | |
| | aaaagcttcatcttggtgagaaaagcagacatggctgatatcccaggccacgtcttcacaagaggaatgatcatagctg | |
| | ttttgatcttggtggcagtagtgtgcctagtgactgtgtgtgtcatttatagagttgacttggttctattttatagacatttaacg | |
| | agaagagatgaaacattaacagatggaaaaacatatgatgcttttgtgtcttacctaaaagaatgccgacctgaaaatgg | |
| | agaggagcacacctttgctgtggagattttgcccagggtgttggagaaacattttgggtataagttatgcatatttgaaag | |
| | ggatgtagtgcctggaggagctgttgttgatgaaatccactcactgatagagaaaagccgaagactaatcattgtcctaa | |
| | gtaaaagttatatgtctaatgaggtcaggtatgaacttgaaagtggactccatgaagcattggtggaaagaaaaattaaa | |
| | ataatcttaattgaatttacacctgttactgacttcacattcttgccccaatcactaaagcttttgaaatctcacagagttctga | |
| | agtggaaggccgataaatctctttcttataactcaaggttctggaagaaccttctttacttaatgcctgcaaaaacagtcaa | |
| | gccaggtagagacgaaccggaagtcttgcctgttctttccgagtctggaagcggtgagggaagaggtagcctgctcac | |
| | gtgcggtgatgtggaggagaaccctgggcctatgctctgtttgggctggatatttctttggcttgttgcaggagagcgaa | |
| | ttaaaggatttaatatttcaggttgttccacaaaaaaactcctttggacatattctacaaggagtgaagaggaatttgtcttat | |
| | tttgtgatttaccagagccacagaaatcacatttctgccacagaaatcgactctcaccaaaacaagtccctgagcacctg | |
| | cccttcatgggtagtaacgacctatctgatgtccaatggtaccaacaaccttcgaatggagatccattagaggacattag | |
| | gaaaagctatcctcacatcattcaggacaaatgtacccttcactttttgaccccaggggtgaataattctgggtcatatattt | |
| | gtagacccaagatgattaagagcccctatgatgtagcctcttgtgtcaagatgattttagaagttaagccccagacaaat | |
| | gcatcctgtgagtattccgcatcacataagcaagacctacttcttgggagcactggctctatttcttgccccagtctcagct | |
| | gccaaagtgatgcacaaagtccagcggtaacctggtacaagaatggaaaactcctctctgtggaaaggagcaaccga | |
| | atcgtagtggatgaagtttatgactatcaccagggcacatatgtatgtgattacactcagtcggatactgtgagttcgtgg | |
| | acagtcagagctgttgttcaagtgagaaccattgtgggagacactaaactcaaaccagatattctggatcctgtcgagga | |
| | cacactggaagtagaacttggaaagcctttaactattagctgcaaagcacgatttggctttgaaagggtctttaaccctgt | |
| | cataaaatggtacatcaaagattctgacctagagtgggaagtctcagtacctgaggcgaaaagtattaaatccactttaa | |
| | aggatgaaatcattgagcgtaatatcatcttggaaaaagtcactcagcgtgatcttcgcaggaagtttgtttgctttgtcca | |
| | gaactccattggaaacacaacccagtccgtccaactgaaagaaaagagaggagtggtgctcctgtacatcctgcttgg | |
| | caccatcgggaccctggtggccgtgctggcggcgagtgccctcctctacaggcactggattgaaatagtgctgctgta | |
| | ccggacctaccagagcaaggatcagacgcttggggataaaaaggattttgatgctttcgtatcctatgcaaaatggagc | |
| | tcttttccaagtgaggccacttcatctctgagtgaagaacacttggccctgagcctatttcctgatgttttagaaaacaaata | |
| | tggatatagcctgtgtttgcttgaaagagatgtggctccaggaggagtgtatgcagaagacattgtgagcattattaaga | |
| | gaagcagaagaggaatatttatcttgagccccaactatgtcaatggacccagtatctttgaactacaagcagcagtgaat | |
| | cttgccttggatgatcaaacactgaaactcattttaattaagttctgttacttccaagagccagagtctctacctcatctcgtg | |
| | aaaaaagctctcagggttttgcccacagttacttggagaggcttaaaatcagttcctcccaattctaggttctgggccaaa | |
| | atgcgctaccacatgcctgtgaaaaactctcagggattcacgtggaaccagctcagaattacctctaggatttttcagtgg | |
| | aaaggactcagtagaacagaaaccactgggaggagctcccagcctaaggaatggtgataa | |
| CAR E6 CD28- | atggccttaccagtgaccgccttgctcctgccgctggccttgctgctccacgccgccaggccggaggtgcagctggtg | 151 |
| CD28-CD28CD3 | gagtctgggggaggcctggtcaagcctggggggtccctgagactctcctgtgcagcctctggattcaccttcagtagg | |
| 3XIL2NFAT | tatggcatgagctgggtccgccaggctccagggaagaggctggagtgggtctcaaccattagtggcggaggcacct | |
| mCMV | acatatactacccagactcagtgaagggccgattcaccatctccagagacaacgccaagaacaccctgtatctgcaaat | |
| IL18Ralpha-T2A- | gaacagcctgagagccgaggacacggctgtgtattactgtaccagagataactatggccgcaactatgattatggcatg | |
| IL18Rb | gattattggggccagggcaccctggtgaccgtgagcagcggtggtggcggttccggcggtggcggttccggtggtg | |
| | gcggttccgaaattgtgttgacacagtctccagccaccctgtctttgtctccaggggaaagagccaccctcacctgcag | |
| | cgccaccagcagtgttagctacatccactggtatcaacagaggcctggccagagccccaggctcctcatctatagcac | |
| | ctccaacctggccagcggcatcccagccaggttcagtggcagtgggtctgggagcgactacactctcaccatcagca | |
| | gcctagagcctgaagattttgcagtttattactgtcagcagcgtagcagctcccctttcacctttggcagcggcaccaaa | |
| | gtggaaattaaaaagcacctgtgtccttctccactgttccccggccctagcaagcctttctgggtcctggtggtggtggg | |
| | cggagtgctggcctgctacagcctgctggtgaccgtggcctttatcatcttctgggtgaggagtaagaggagcaggct | |
| | cctgcacagtgactacatgaacatgactcctagaagacctgggcctaccagaaagcattaccagccctatgccccacc | |
| | acgcgacttcgcagcctatcgctccagagtgaagttcagcaggagcgcagacgcccccgcgtacaagcagggcca | |
| | gaaccagctctataacgagctcaatctaggacgaagagaggagtacgatgttttggacaagagacgtggccgggacc | |
| | ctgagatggggggaaagccgagaaggaagaaccctcaggaaggcctgtacaatgaactgcagaaagataagatgg | |
| | cggaggcctacagtgagattgggatgaaaggcgagcgccggaggggcaaggggcacgatggcctttaccagggtc | |
| | tcagtacagccaccaaggacacctacgacgcccttcacatgcaggccctgccccctcgctgataagtttaaactgcca | |
| | gaacatttctctggcctaactggccggtaccggaggaaaaactgtttcatacagaaggcgtggaggaaaaactgtttca | |
| | tacagaaggcgtggaggaaaaactgtttcatacagaaggcgtactagttaggcgtgtacggtgggaggcctatataag | |
| | cagagctcgtttagtgaaccgtcagatcgcctggagacgccatccacgctgttttgacctccatagaagacaccggga | |
| | ccgatccagcctcgagagacccaatgctagccaccatgaattgtagagaattacccttgaccctttgggtgcttatatct | |
| | gtaagcactgcagaatcttgtacttcacgtccccacattactgtggttgaaggggaacctttctatctgaaacattgctcgt | |
| | gttcacttgcacatgagattgaaacaaccaccaaaagctggtacaaaagcagtggatcacaggaacatgtggagctga | |
| | acccaaggagttccagcagaattgctttgcatgattgtgttttggagttttggccagttgagttgaatgacacaggatctta | |
| | ctttttccaaatgaaaaattatactcagaaatggaaattaaatgtcatcagaagaaataaacacagctgtttcactgaaaga | |
| | caagtaactagtaaaattgtggaagttaaaaaattttttcagataacctgtgaaaacagttactatcaaacactggtcaaca | |
| | gcacatcattgtataagaactgtaaaaagctactactggagaacaataaaaacccaacgataaagaagaacgccgagt | |
| | ttgaagatcaggggtattactcctgcgtgcatttccttcatcataatggaaaactatttaatatcaccaaaaccttcaatataa | |
| | caatagtggaagatcgcagtaatatagttccggttcttcttggaccaaagcttaaccatgttgcagtggaattaggaaaaa | |
| | acgtaaggctcaactgctctgctttgctgaatgaagaggatgtaatttattggatgttcggggaagaaaatggatcggat | |
| | cctaatatacatgaagagaaagaaatgagaattatgactccagaaggcaaatggcatgcttcaaaagtattgagaattga | |
| | aaatattggtgaaagcaatctaaatgttttatataattgcactgtggccagcacgggaggcacagacaccaaaagcttca | |
| | tcttggtgagaaaagcagacatggctgatatcccaggccacgtcttcacaagaggaatgatcatagctgttttgatcttgg | |
| | tggcagtagtgtgcctagtgactgtgtgtgtcatttatagagttgacttggttctattttatagacatttaacgagaagagatg | |
| | aaacattaacagatggaaaaacatatgatgcttttgtgtcttacctaaaagaatgccgacctgaaaatggagaggagcac | |
| | acctttgctgtggagattttgcccagggtgttggagaaacattttgggtataagttatgcatatttgaaagggatgtagtgc | |
| | ctggaggagctgttgttgatgaaatccactcactgatagagaaaagccgaagactaatcattgtcctaagtaaaagttata | |
| | tgtctaatgaggtcaggtatgaacttgaaagtggactccatgaagcattggtggaaagaaaaattaaaataatcttaattg | |
| | aatttacacctgttactgacttcacattcttgccccaatcactaaagcttttgaaatctcacagagttctgaagtggaaggcc | |
| | gataaatctctttcttataactcaaggttctggaagaaccttctttacttaatgcctgcaaaaacagtcaagccaggtagag | |
| | acgaaccggaagtcttgcctgttctttccgagtctggaagcggtgagggaagaggtagcctgctcacgtgcggtgatg | |
| | tggaggagaaccctgggcctatgctctgtttgggctggatatttctttggcttgttgcaggagagcgaattaaaggattta | |
| | atatttcaggttgttccacaaaaaaactcctttggacatattctacaaggagtgaagaggaatttgtcttattttgtgatttacc | |
| | agagccacagaaatcacatttctgccacagaaatcgactctcaccaaaacaagtccctgagcacctgcccttcatgggt | |
| | agtaacgacctatctgatgtccaatggtaccaacaaccttcgaatggagatccattagaggacattaggaaaagctatcc | |
| | tcacatcattcaggacaaatgtacccttcactttttgaccccaggggtgaataattctgggtcatatatttgtagacccaag | |
| | atgattaagagcccctatgatgtagcctgttgtgtcaagatgattttagaagttaagccccagacaaatgcatcctgtgagt | |
| | attccgcatcacataagcaagacctacttcttgggagcactggctctatttcttgccccagtctcagctgccaaagtgatg | |
| | cacaaagtccagcggtaacctggtacaagaatggaaaactcctctctgtggaaaggagcaaccgaatcgtagtggatg | |
| | aagtttatgactatcaccagggcacatatgtatgtgattacactcagtcggatactgtgagttcgtggacagtcagagctg | |
| | ttgttcaagtgagaaccattgtgggagacactaaactcaaaccagatattctggatcctgtcgaggacacactggaagta | |
| | gaacttggaaagcctttaactattagctgcaaagcacgatttggctttgaaagggtctttaaccctgtcataaaatggtaca | |
| | tcaaagattctgacctagagtgggaagtctcagtacctgaggcgaaaagtattaaatccactttaaaggatgaaatcattg | |
| | agcgtaatatcatcttggaaaaagtcactcagcgtgatcttcgcaggaagtttgtttgctttgtccagaactccattggaaa | |
| | cacaacccagtccgtccaactgaaagaaaagagaggagtggtgctcctgtacatcctgcttggcaccatcgggaccct | |
| | ggtggccgtgctggcggcgagtgccctcctctacaggcactggattgaaatagtgctgctgtaccggacctaccagag | |
| | caaggatcagacgcttggggataaaaaggattttgatgctttcgtatcctatgcaaaatggagctcttttccaagtgaggc | |
| | cacttcatctctgagtgaagaacacttggccctgagcctatttcctgatgttttagaaaacaaatatggatatagcctgtgtt | |
| | tgcttgaaagagatgtggctccaggaggagtgtatgcagaagacattgtgagcattattaagagaagcagaagaggaa | |
| | tatttatcttgagccccaactatgtcaatggacccagtatctttgaactacaagcagcagtgaatcttgccttggatgatca | |
| | aacactgaaactcattttaattaagttctgttacttccaagagccagagtctctacctcatctcgtgaaaaaagctctcagg | |
| | gttttgcccacagttacttggagaggcttaaaatcagttcctcccaattctaggttctgggccaaaatgcgctaccacatg | |
| | cctgtgaaaaactctcagggattcacgtggaaccagctcagaattacctctaggatttttcagtggaaaggactcagtag | |
| | aacagaaaccactgggaggagctcccagcctaaggaatggtgataa | |
| CAR E6 CD28- | atggccttaccagtgaccgccttgctcctgccgctggccttgctgctccacgccgccaggccggaggtgcagctggtg | 152 |
| CD28-CD281XX | gagtctgggggaggcctggtcaagcctggggggtccctgagactctcctgtgcagcctctggattcaccttcagtagg | |
| 6XFOXP3NFAT | tatggcatgagctgggtccgccaggctccagggaagaggctggagtgggtctcaaccattagtggcggaggcacct | |
| mCMV | acatatactacccagactcagtgaagggccgattcaccatctccagagacaacgccaagaacaccctgtatctgcaaat | |
| IL18Ralpha-T2A- | gaacagcctgagagccgaggacacggctgtgtattactgtaccagagataactatggccgcaactatgattatggcatg | |
| IL18Rb | gattattggggccagggcaccctggtgaccgtgagcagcggtggtggcggttccggcggtggcggttccggtggtg | |
| | gcggttccgaaattgtgttgacacagtctccagccaccctgtctttgtctccaggggaaagagccaccctcacctgcag | |
| | cgccaccagcagtgttagctacatccactggtatcaacagaggcctggccagagccccaggctcctcatctatagcac | |
| | ctccaacctggccagcggcatcccagccaggttcagtggcagtgggtctgggagcgactacactctcaccatcagca | |
| | gcctagagcctgaagattttgcagtttattactgtcagcagcgtagcagctcccctttcacctttggcagcggcaccaaa | |
| | gtggaaattaaaaagcacctgtgtccttctccactgttccccggccctagcaagcctttctgggtcctggtggtggtggg | |
| | cggagtgctggcctgctacagcctgctggtgaccgtggcctttatcatcttctgggtgaggagtaagaggagcaggct | |
| | cctgcacagtgactacatgaacatgactcctagaagacctgggcctaccagaaagcattaccagccctatgccccacc | |
| | acgcgacttcgcagcctatcgctccagagtgaagttcagcaggagcgcagacgcccccgcgtacaagcagggcca | |
| | gaaccagctctataacgagctcaatctaggacgaagagaggagtacgatgttttggacaagagacgtggccgggacc | |
| | ctgagatggggggaaagccgagaaggaagaaccctcaggaaggcctgttcaatgaactgcagaaagataagatgg | |
| | cggaggccttcagtgagattgggatgaaaggcgagcgccggaggggcaaggggcacgatggccttttccagggtct | |
| | cagtacagccaccaaggacaccttcgacgcccttcacatgcaggccctgccccctcgctgataagtttaaactgccag | |
| | aacattactctggcctaactggccggtaccggcttcattttttccatttactgcagaggcttcattttttccatttactgcagag | |
| | gcttcattttttccatttactgcagaggcttcattttttccatttactgcagaggcttcattttttccatttactgcagaggcttcat | |
| | tttttccatttactgcagaactagttaggcgtgtacggtgggaggcctatataagcagagctcgtttagtgaaccgtcaga | |
| | tcgcctggagacgccatccacgctgttttgacctccatagaagacaccgggaccgatccagcctcgagagacccaat | |
| | gctagccaccatgaattgtagagaattacccttgaccctttgggtgcttatatctgtaagcactgcagaatcttgtacttca | |
| | cgtccccacattactgtggttgaaggggaacctttctatctgaaacattgctcgtgttcacttgcacatgagattgaaacaa | |
| | ccaccaaaagctggtacaaaagcagtggatcacaggaacatgtggagctgaacccaaggagttccagcagaattgct | |
| | ttgcatgattgtgttttggagttttggccagttgagttgaatgacacaggatcttactttttccaaatgaaaaattatactcaga | |
| | aatggaaattaaatgtcatcagaagaaataaacacagctgtttcactgaaagacaagtaactagtaaaattgtggaagtta | |
| | aaaaattttttcagataacctgtgaaaacagttactatcaaacactggtcaacagcacatcattgtataagaactgtaaaaa | |
| | gctactactggagaacaataaaaacccaacgataaagaagaacgccgagtttgaagatcaggggtattactcctgcgt | |
| | gcatttccttcatcataatggaaaactatttaatatcaccaaaaccttcaatataacaatagtggaagatcgcagtaatatag | |
| | ttccggttcttcttggaccaaagcttaaccatgttgcagtggaattaggaaaaaacgtaaggctcaactgctctgctttgct | |
| | gaatgaagaggatgtaatttattggatgttcggggaagaaaatggatcggatcctaatatacatgaagagaaagaaatg | |
| | agaattatgactccagaaggcaaatggcatgcttcaaaagtattgagaattgaaaatattggtgaaagcaatctaaatgtt | |
| | ttatataattgcactgtggccagcacgggaggcacagacaccaaaagcttcatcttggtgagaaaagcagacatggct | |
| | gatatcccaggccacgtcttcacaagaggaatgatcatagctgttttgatcttggtggcagtagtgtgcctagtgactgtg | |
| | tgtgtcatttatagagttgacttggttctattttatagacatttaacgagaagagatgaaacattaacagatggaaaaacata | |
| | tgatgcttttgtgtcttacctaaaagaatgccgacctgaaaatggagaggagcacacctttgctgtggagattttgcccag | |
| | ggtgttggagaaacattttgggtataagttatgcatatttgaaagggatgtagtgcctggaggagctgttgttgatgaaatc | |
| | cactcactgatagagaaaagccgaagactaatcattgtcctaagtaaaagttatatgtctaatgaggtcaggtatgaactt | |
| | gaaagtggactccatgaagcattggtggaaagaaaaattaaaataatcttaattgaatttacacctgttactgacttcacatt | |
| | cttgccccaatcactaaagcttttgaaatctcacagagttctgaagtggaaggccgataaatctctttcttataactcaaggt | |
| | tctggaagaaccttctttacttaatgcctgcaaaaacagtcaagccaggtagagacgaaccggaagtcttgcctgttcttt | |
| | ccgagtctggaagcggtgagggaagaggtagcctgctcacgtgcggtgatgtggaggagaaccctgggcctatgct | |
| | ctgtttgggctggatatttctttggcttgttgcaggagagcgaattaaaggatttaatatttcaggttgttccacaaaaaaact | |
| | cctttggacatattctacaaggagtgaagaggaatttgtcttattttgtgatttaccagagccacagaaatcacatttctgcc | |
| | acagaaatcgactctcaccaaaacaagtccctgagcacctgcccttcatgggtagtaacgacctatctgatgtccaatg | |
| | gtaccaacaaccttcgaatggagatccattagaggacattaggaaaagctatcctcacatcattcaggacaaatgtaccc | |
| | ttcactttttgaccccaggggtgaataattctgggtcatatatttgtagacccaagatgattaagagcccctatgatgtagc | |
| | ctgttgtgtcaagatgattttagaagttaagccccagacaaatgcatcctgtgagtattccgcatcacataagcaagacct | |
| | acttcttgggagcactggctctatttcttgccccagtctcagctgccaaagtgatgcacaaagtccagcggtaacctggt | |
| | acaagaatggaaaactcctctctgtggaaaggagcaaccgaatcgtagtggatgaagtttatgactatcaccagggca | |
| | catatgtatgtgattacactcagtcggatactgtgagttcgtggacagtcagagctgttgttcaagtgagaaccattgtgg | |
| | gagacactaaactcaaaccagatattctggatcctgtcgaggacacactggaagtagaacttggaaagcctttaactatt | |
| | agctgcaaagcacgatttggctttgaaagggtctttaaccctgtcataaaatggtacatcaaagattctgacctagagtgg | |
| | gaagtctcagtacctgaggcgaaaagtattaaatccactttaaaggatgaaatcattgagcgtaatatcatcttggaaaaa | |
| | gtcactcagcgtgatcttcgcaggaagtttgtttgctttgtccagaactccattggaaacacaacccagtccgtccaactg | |
| | aaagaaaagagaggagtggtgctcctgtacatcctgcttggcaccatcgggaccctggtggccgtgctggcggcgag | |
| | tgccctcctctacaggcactggattgaaatagtgctgctgtaccggacctaccagagcaaggatcagacgcttggggat | |
| | aaaaaggattttgatgctttcgtatcctatgcaaaatggagctcttttccaagtgaggccacttcatctctgagtgaagaac | |
| | acttggccctgagcctatttcctgatgttttagaaaacaaatatggatatagcctgtgtttgcttgaaagagatgtggctcca | |
| | ggaggagtgtatgcagaagacattgtgagcattattaagagaagcagaagaggaatatttatcttgagccccaactatgt | |
| | caatggacccagtatctttgaactacaagcagcagtgaatcttgccttggatgatcaaacactgaaactcattttaattaag | |
| | ttctgttacttccaagagccagagtctctacctcatctcgtgaaaaaagctctcagggttttgcccacagttacttggagag | |
| | gcttaaaatcagttcctcccaattctaggttctgggccaaaatgcgctaccacatgcctgtgaaaaactctcagggattca | |
| | cgtggaaccagctcagaattacctctaggatttttcagtggaaaggactcagtagaacagaaaccactgggaggagct | |
| | cccagcctaaggaatggtgataa | |
| CAR E6 CD28- | atggccttaccagtgaccgccttgctcctgccgctggccttgctgctccacgccgccaggccggaggtgcagctggtg | 153 |
| CD28-CD281XX | gagtctgggggaggcctggtcaagcctggggggtccctgagactctcctgtgcagcctctggattcaccttcagtagg | |
| 3XFOXP3NFAT | tatggcatgagctgggtccgccaggctccagggaagaggctggagtgggtctcaaccattagtggcggaggcacct | |
| mCMV | acatatactacccagactcagtgaagggccgattcaccatctccagagacaacgccaagaacaccctgtatctgcaaat | |
| IL18Ralpha-T2A- | gaacagcctgagagccgaggacacggctgtgtattactgtaccagagataactatggccgcaactatgattatggcatg | |
| IL18Rb | gattattggggccagggcaccctggtgaccgtgagcagcggtggtggcggttccggcggtggcggttccggtggtg | |
| | gcggttccgaaattgtgttgacacagtctccagccaccctgtctttgtctccaggggaaagagccaccctcacctgcag | |
| | cgccaccagcagtgttagctacatccactggtatcaacagaggcctggccagagccccaggctcctcatctatagcac | |
| | ctccaacctggccagcggcatcccagccaggttcagtggcagtgggtctgggagcgactacactctcaccatcagca | |
| | gcctagagcctgaagattttgcagtttattactgtcagcagcgtagcagctcccctttcacctttggcagcggcaccaaa | |
| | gtggaaattaaaaagcacctgtgtccttctccactgttccccggccctagcaagcctttctgggtcctggtggtggtggg | |
| | cggagtgctggcctgctacagcctgctggtgaccgtggcctttatcatcttctgggtgaggagtaagaggagcaggct | |
| | cctgcacagtgactacatgaacatgactcctagaagacctgggcctaccagaaagcattaccagccctatgccccacc | |
| | acgcgacttcgcagcctatcgctccagagtgaagttcagcaggagcgcagacgcccccgcgtacaagcagggcca | |
| | gaaccagctctataacgagctcaatctaggacgaagagaggagtacgatgttttggacaagagacgtggccgggacc | |
| | ctgagatggggggaaagccgagaaggaagaaccctcaggaaggcctgttcaatgaactgcagaaagataagatgg | |
| | cggaggccttcagtgagattgggatgaaaggcgagcgccggaggggcaaggggcacgatggccttttccagggtct | |
| | cagtacagccaccaaggacaccttcgacgcccttcacatgcaggccctgccccctcgctgataagtttaaactgccag | |
| | aacattactctggcctaactggccggtaccggcttcattttttccatttactgcagaggcttcattttttccatttactgcagag | |
| | gcttcattttttccatttactgcagaactagttaggcgtgtacggtgggaggcctatataagcagagctcgtttagtgaacc | |
| | gtcagatcgcctggagacgccatccacgctgttttgacctccatagaagacaccgggaccgatccagcctcgagaga | |
| | cccaatgctagccaccatgaattgtagagaattacccttgaccctttgggtgcttatatctgtaagcactgcagaatcttgt | |
| | acttcacgtccccacattactgtggttgaaggggaacctttctatctgaaacattgctcgtgttcacttgcacatgagattga | |
| | aacaaccaccaaaagctggtacaaaagcagtggatcacaggaacatgtggagctgaacccaaggagttccagcaga | |
| | attgctttgcatgattgtgttttggagttttggccagttgagttgaatgacacaggatcttactttttccaaatgaaaaattata | |
| | ctcagaaatggaaattaaatgtcatcagaagaaataaacacagctgtttcactgaaagacaagtaactagtaaaattgtg | |
| | gaagttaaaaaattttttcagataacctgtgaaaacagttactatcaaacactggtcaacagcacatcattgtataagaact | |
| | gtaaaaagctactactggagaacaataaaaacccaacgataaagaagaacgccgagtttgaagatcaggggtattact | |
| | cctgcgtgcatttccttcatcataatggaaaactatttaatatcaccaaaaccttcaatataacaatagtggaagatcgcagt | |
| | aatatagttccggttcttcttggaccaaagcttaaccatgttgcagtggaattaggaaaaaacgtaaggctcaactgctct | |
| | gctttgctgaatgaagaggatgtaatttattggatgttcggggaagaaaatggatcggatcctaatatacatgaagagaa | |
| | agaaatgagaattatgactccagaaggcaaatggcatgcttcaaaagtattgagaattgaaaatattggtgaaagcaatc | |
| | taaatgttttatataattgcactgtggccagcacgggaggcacagacaccaaaagcttcatcttggtgagaaaagcaga | |
| | catggctgatatcccaggccacgtcttcacaagaggaatgatcatagctgttttgatcttggtggcagtagtgtgcctagt | |
| | gactgtgtgtgtcatttatagagttgacttggttctattttatagacatttaacgagaagagatgaaacattaacagatggaa | |
| | aaacatatgatgcttttgtgtcttacctaaaagaatgccgacctgaaaatggagaggagcacacctttgctgtggagatttt | |
| | gcccagggtgttggagaaacattttgggtataagttatgcatatttgaaagggatgtagtgcctggaggagctgttgttga | |
| | tgaaatccactcactgatagagaaaagccgaagactaatcattgtcctaagtaaaagttatatgtctaatgaggtcaggta | |
| | tgaacttgaaagtggactccatgaagcattggtggaaagaaaaattaaaataatcttaattgaatttacacctgttactgac | |
| | ttcacattcttgccccaatcactaaagcttttgaaatctcacagagttctgaagtggaaggccgataaatctctttcttataac | |
| | tcaaggttctggaagaaccttctttacttaatgcctgcaaaaacagtcaagccaggtagagacgaaccggaagtcttgc | |
| | ctgttctttccgagtctggaagcggtgagggaagaggtagcctgctcacgtgcggtgatgtggaggagaaccctggg | |
| | cctatgctctgtttgggctggatatttctttggcttgttgcaggagagcgaattaaaggatttaatatttcaggttgttccaca | |
| | aaaaaactcctttggacatattctacaaggagtgaagaggaatttgtcttattttgtgatttaccagagccacagaaatcac | |
| | atttctgccacagaaatcgactctcaccaaaacaagtccctgagcacctgcccttcatgggtagtaacgacctatctgat | |
| | gtccaatggtaccaacaaccttcgaatggagatccattagaggacattaggaaaagctatcctcacatcattcaggaca | |
| | aatgtacccttcactttttgaccccaggggtgaataattctgggtcatatatttgtagacccaagatgattaagagcccctat | |
| | gatgtagcctgttgtgtcaagatgattttagaagttaagccccagacaaatgcatcctgtgagtattccgcatcacataag | |
| | caagacctacttcttgggagcactggctctatttcttgccccagtctcagctgccaaagtgatgcacaaagtccagcggt | |
| | aacctggtacaagaatggaaaactcctctctgtggaaaggagcaaccgaatcgtagtggatgaagtttatgactatcac | |
| | cagggcacatatgtatgtgattacactcagtcggatactgtgagttcgtggacagtcagagctgttgttcaagtgagaac | |
| | cattgtgggagacactaaactcaaaccagatattctggatcctgtcgaggacacactggaagtagaacttggaaagcct | |
| | ttaactattagctgcaaagcacgatttggctttgaaagggtctttaaccctgtcataaaatggtacatcaaagattctgacct | |
| | agagtgggaagtctcagtacctgaggcgaaaagtattaaatccactttaaaggatgaaatcattgagcgtaatatcatctt | |
| | ggaaaaagtcactcagcgtgatcttcgcaggaagtttgtttgctttgtccagaactccattggaaacacaacccagtccgt | |
| | ccaactgaaagaaaagagaggagtggtgctcctgtacatcctgcttggcaccatcgggaccctggtggccgtgctgg | |
| | cggcgagtgccctcctctacaggcactggattgaaatagtgctgctgtaccggacctaccagagcaaggatcagacg | |
| | cttggggataaaaaggattttgatgctttcgtatcctatgcaaaatggagctcttttccaagtgaggccacttcatctctgag | |
| | tgaagaacacttggccctgagcctatttcctgatgttttagaaaacaaatatggatatagcctgtgtttgcttgaaagagat | |
| | gtggctccaggaggagtgtatgcagaagacattgtgagcattattaagagaagcagaagaggaatatttatcttgagcc | |
| | ccaactatgtcaatggacccagtatctttgaactacaagcagcagtgaatcttgccttggatgatcaaacactgaaactca | |
| | ttttaattaagttctgttacttccaagagccagagtctctacctcatctcgtgaaaaaagctctcagggttttgcccacagtta | |
| | cttggagaggcttaaaatcagttcctcccaattctaggttctgggccaaaatgcgctaccacatgcctgtgaaaaactctc | |
| | agggattcacgtggaaccagctcagaattacctctaggatttttcagtggaaaggactcagtagaacagaaaccactgg | |
| | gaggagctcccagcctaaggaatggtgataa | |
| CAR E6 CD28- | atggccttaccagtgaccgccttgctcctgccgctggccttgctgctccacgccgccaggccggaggtgcagctggtg | 154 |
| CD28-CD281XX | gagtctgggggaggcctggtcaagcctggggggtccctgagactctcctgtgcagcctctggattcaccttcagtagg | |
| 6XIL2NFAT | tatggcatgagctgggtccgccaggctccagggaagaggctggagtgggtctcaaccattagtggcggaggcacct | |
| mCMV | acatatactacccagactcagtgaagggccgattcaccatctccagagacaacgccaagaacaccctgtatctgcaaat | |
| IL18Ralpha-T2A- | gaacagcctgagagccgaggacacggctgtgtattactgtaccagagataactatggccgcaactatgattatggcatg | |
| IL18Rb | gattattggggccagggcaccctggtgaccgtgagcagcggtggtggcggttccggcggtggcggttccggtggtg | |
| | gcggttccgaaattgtgttgacacagtctccagccaccctgtctttgtctccaggggaaagagccaccctcacctgcag | |
| | cgccaccagcagtgttagctacatccactggtatcaacagaggcctggccagagccccaggctcctcatctatagcac | |
| | ctccaacctggccagcggcatcccagccaggttcagtggcagtgggtctgggagcgactacactctcaccatcagca | |
| | gcctagagcctgaagattttgcagtttattactgtcagcagcgtagcagctcccctttcacctttggcagcggcaccaaa | |
| | gtggaaattaaaaagcacctgtgtccttctccactgttccccggccctagcaagcctttctgggtcctggtggtggtggg | |
| | cggagtgctggcctgctacagcctgctggtgaccgtggcctttatcatcttctgggtgaggagtaagaggagcaggct | |
| | cctgcacagtgactacatgaacatgactcctagaagacctgggcctaccagaaagcattaccagccctatgccccacc | |
| | acgcgacttcgcagcctatcgctccagagtgaagttcagcaggagcgcagacgcccccgcgtacaagcagggcca | |
| | gaaccagctctataacgagctcaatctaggacgaagagaggagtacgatgttttggacaagagacgtggccgggacc | |
| | ctgagatggggggaaagccgagaaggaagaaccctcaggaaggcctgttcaatgaactgcagaaagataagatgg | |
| | cggaggccttcagtgagattgggatgaaaggcgagcgccggaggggcaaggggcacgatggccttttccagggtct | |
| | cagtacagccaccaaggacaccttcgacgcccttcacatgcaggccctgccccctcgctgataagtttaaactgccag | |
| | aacattactctggcctaactggccggtaccggaggaaaaactgtttcatacagaaggcgtggaggaaaaactgtttcat | |
| | acagaaggcgtggaggaaaaactgtttcatacagaaggcgtggaggaaaaactgtttcatacagaaggcgtggagga | |
| | aaaactgtttcatacagaaggcgtggaggaaaaactgtttcatacagaaggcgtactagttaggcgtgtacggtgggag | |
| | gcctatataagcagagctcgtttagtgaaccgtcagatcgcctggagacgccatccacgctgttttgacctccatagaag | |
| | acaccgggaccgatccagcctcgagagacccaatgctagccaccatgaattgtagagaattacccttgaccctttggg | |
| | tgcttatatctgtaagcactgcagaatcttgtacttcacgtccccacattactgtggttgaaggggaacctttctatctgaaa | |
| | cattgctcgtgttcacttgcacatgagattgaaacaaccaccaaaagctggtacaaaagcagtggatcacaggaacatg | |
| | tggagctgaacccaaggagttccagcagaattgctttgcatgattgtgttttggagttttggccagttgagttgaatgacac | |
| | aggatcttactttttccaaatgaaaaattatactcagaaatggaaattaaatgtcatcagaagaaataaacacagctgittca | |
| | ctgaaagacaagtaactagtaaaattgtggaagttaaaaaattttttcagataacctgtgaaaacagttactatcaaacact | |
| | ggtcaacagcacatcattgtataagaactgtaaaaagctactactggagaacaataaaaacccaacgataaagaagaa | |
| | cgccgagtttgaagatcaggggtattactcctgcgtgcatttccttcatcataatggaaaactatttaatatcaccaaaacct | |
| | tcaatataacaatagtggaagatcgcagtaatatagttccggttcttcttggaccaaagcttaaccatgttgcagtggaatt | |
| | aggaaaaaacgtaaggctcaactgctctgctttgctgaatgaagaggatgtaatttattggatgttcggggaagaaaatg | |
| | gatcggatcctaatatacatgaagagaaagaaatgagaattatgactccagaaggcaaatggcatgcttcaaaagtatt | |
| | gagaattgaaaatattggtgaaagcaatctaaatgttttatataattgcactgtggccagcacgggaggcacagacacca | |
| | aaagcttcatcttggtgagaaaagcagacatggctgatatcccaggccacgtcttcacaagaggaatgatcatagctgtt | |
| | ttgatcttggtggcagtagtgtgcctagtgactgtgtgtgtcatttatagagttgacttggttctattttatagacatttaacga | |
| | gaagagatgaaacattaacagatggaaaaacatatgatgcttttgtgtcttacctaaaagaatgccgacctgaaaatgga | |
| | gaggagcacacctttgctgtggagattttgcccagggtgttggagaaacattttgggtataagttatgcatatttgaaagg | |
| | gatgtagtgcctggaggagctgttgttgatgaaatccactcactgatagagaaaagccgaagactaatcattgtcctaag | |
| | taaaagttatatgtctaatgaggtcaggtatgaacttgaaagtggactccatgaagcattggtggaaagaaaaattaaaat | |
| | aatcttaattgaatttacacctgttactgacttcacattcttgccccaatcactaaagcttttgaaatctcacagagttctgaag | |
| | tggaaggccgataaatctctttcttataactcaaggttctggaagaaccttctttacttaatgcctgcaaaaacagtcaagc | |
| | caggtagagacgaaccggaagtcttgcctgttctttccgagtctggaagcggtgagggaagaggtagcctgctcacgt | |
| | gcggtgatgtggaggagaaccctgggcctatgctctgtttgggctggatatttctttggcttgttgcaggagagcgaatta | |
| | aaggatttaatatttcaggttgttccacaaaaaaactcctttggacatattctacaaggagtgaagaggaatttgtcttatttt | |
| | gtgatttaccagagccacagaaatcacatttctgccacagaaatcgactctcaccaaaacaagtccctgagcacctgcc | |
| | cttcatgggtagtaacgacctatctgatgtccaatggtaccaacaaccttcgaatggagatccattagaggacattagga | |
| | aaagctatcctcacatcattcaggacaaatgtacccttcactttttgaccccaggggtgaataattctgggtcatatatttgt | |
| | agacccaagatgattaagagcccctatgatgtagcctgttgtgtcaagatgattttagaagttaagccccagacaaatgc | |
| | atcctgtgagtattccgcatcacataagcaagacctacttcttgggagcactggctctatttcttgccccagtctcagctgc | |
| | caaagtgatgcacaaagtccagcggtaacctggtacaagaatggaaaactcctctctgtggaaaggagcaaccgaat | |
| | cgtagtggatgaagtttatgactatcaccagggcacatatgtatgtgattacactcagtcggatactgtgagttcgtggac | |
| | agtcagagctgttgttcaagtgagaaccattgtgggagacactaaactcaaaccagatattctggatcctgtcgaggaca | |
| | cactggaagtagaacttggaaagcctttaactattagctgcaaagcacgatttggctttgaaagggtctttaaccctgtcat | |
| | aaaatggtacatcaaagattctgacctagagtgggaagtctcagtacctgaggcgaaaagtattaaatccactttaaagg | |
| | atgaaatcattgagcgtaatatcatcttggaaaaagtcactcagcgtgatcttcgcaggaagtttgtttgctttgtccagaa | |
| | ctccattggaaacacaacccagtccgtccaactgaaagaaaagagaggagtggtgctcctgtacatcctgcttggcac | |
| | catcgggaccctggtggccgtgctggcggcgagtgccctcctctacaggcactggattgaaatagtgctgctgtaccg | |
| | gacctaccagagcaaggatcagacgcttggggataaaaaggattttgatgctttcgtatcctatgcaaaatggagctcttt | |
| | tccaagtgaggccacttcatctctgagtgaagaacacttggccctgagcctatttcctgatgttttagaaaacaaatatgg | |
| | atatagcctgtgtttgcttgaaagagatgtggctccaggaggagtgtatgcagaagacattgtgagcattattaagagaa | |
| | gcagaagaggaatatttatcttgagccccaactatgtcaatggacccagtatctttgaactacaagcagcagtgaatcttg | |
| | ccttggatgatcaaacactgaaactcattttaattaagttctgttacttccaagagccagagtctctacctcatctcgtgaaa | |
| | aaagctctcagggttttgcccacagttacttggagaggcttaaaatcagttcctcccaattctaggttctgggccaaaatg | |
| | cgctaccacatgcctgtgaaaaactctcagggattcacgtggaaccagctcagaattacctctaggatttttcagtggaa | |
| | aggactcagtagaacagaaaccactgggaggagctcccagcctaaggaatggtgataa | |
| CAR E6 CD28- | atggccttaccagtgaccgccttgctcctgccgctggccttgctgctccacgccgccaggccggaggtgcagctggtg | 155 |
| CD28-CD281XX | gagtctgggggaggcctggtcaagcctggggggtccctgagactctcctgtgcagcctctggattcaccttcagtagg | |
| 3XIL2NFAT | tatggcatgagctgggtccgccaggctccagggaagaggctggagtgggtctcaaccattagtggcggaggcacct | |
| mCMV | acatatactacccagactcagtgaagggccgattcaccatctccagagacaacgccaagaacaccctgtatctgcaaat | |
| IL18Ralpha-T2A- | gaacagcctgagagccgaggacacggctgtgtattactgtaccagagataactatggccgcaactatgattatggcatg | |
| IL18Rb | gattattggggccagggcaccctggtgaccgtgagcagcggtggtggcggttccggcggtggcggttccggtggtg | |
| | gcggttccgaaattgtgttgacacagtctccagccaccctgtctttgtctccaggggaaagagccaccctcacctgcag | |
| | cgccaccagcagtgttagctacatccactggtatcaacagaggcctggccagagccccaggctcctcatctatagcac | |
| | ctccaacctggccagcggcatcccagccaggttcagtggcagtgggtctgggagcgactacactctcaccatcagca | |
| | gcctagagcctgaagattttgcagtttattactgtcagcagcgtagcagctcccctttcacctttggcagcggcaccaaa | |
| | gtggaaattaaaaagcacctgtgtccttctccactgttccccggccctagcaagcctttctgggtcctggtggtggtggg | |
| | cggagtgctggcctgctacagcctgctggtgaccgtggcctttatcatcttctgggtgaggagtaagaggagcaggct | |
| | cctgcacagtgactacatgaacatgactcctagaagacctgggcctaccagaaagcattaccagccctatgccccacc | |
| | acgcgacttcgcagcctatcgctccagagtgaagttcagcaggagcgcagacgcccccgcgtacaagcagggcca | |
| | gaaccagctctataacgagctcaatctaggacgaagagaggagtacgatgttttggacaagagacgtggccgggacc | |
| | ctgagatggggggaaagccgagaaggaagaaccctcaggaaggcctgttcaatgaactgcagaaagataagatgg | |
| | cggaggccttcagtgagattgggatgaaaggcgagcgccggaggggcaaggggcacgatggccttttccagggtct | |
| | cagtacagccaccaaggacaccttcgacgcccttcacatgcaggccctgccccctcgctgataagtttaaactgccag | |
| | aacattactctggcctaactggccggtaccggaggaaaaactgtttcatacagaaggcgtggaggaaaaactgtttcat | |
| | acagaaggcgtggaggaaaaactgtttcatacagaaggcgtactagttaggcgtgtacggtgggaggcctatataagc | |
| | agagctcgtttagtgaaccgtcagatcgcctggagacgccatccacgctgttttgacctccatagaagacaccgggac | |
| | cgatccagcctcgagagacccaatgctagccaccatgaattgtagagaattacccttgaccctttgggtgcttatatctg | |
| | taagcactgcagaatcttgtacttcacgtccccacattactgtggttgaaggggaacctttctatctgaaacattgctcgtgt | |
| | tcacttgcacatgagattgaaacaaccaccaaaagctggtacaaaagcagtggatcacaggaacatgtggagctgaac | |
| | ccaaggagttccagcagaattgctttgcatgattgtgttttggagttttggccagttgagttgaatgacacaggatcttactt | |
| | tttccaaatgaaaaattatactcagaaatggaaattaaatgtcatcagaagaaataaacacagctgtttcactgaaagaca | |
| | agtaactagtaaaattgtggaagttaaaaaattttttcagataacctgtgaaaacagttactatcaaacactggtcaacagc | |
| | acatcattgtataagaactgtaaaaagctactactggagaacaataaaaacccaacgataaagaagaacgccgagtttg | |
| | aagatcaggggtattactcctgcgtgcatttccttcatcataatggaaaactatttaatatcaccaaaaccttcaatataaca | |
| | atagtggaagatcgcagtaatatagttccggttcttcttggaccaaagcttaaccatgttgcagtggaattaggaaaaaac | |
| | gtaaggctcaactgctctgctttgctgaatgaagaggatgtaatttattggatgttcggggaagaaaatggatcggatcct | |
| | aatatacatgaagagaaagaaatgagaattatgactccagaaggcaaatggcatgcttcaaaagtattgagaattgaaa | |
| | atattggtgaaagcaatctaaatgttttatataattgcactgtggccagcacgggaggcacagacaccaaaagcttcatct | |
| | tggtgagaaaagcagacatggctgatatcccaggccacgtcttcacaagaggaatgatcatagctgttttgatcttggtg | |
| | gcagtagtgtgcctagtgactgtgtgtgtcatttatagagttgacttggttctattttatagacatttaacgagaagagatga | |
| | aacattaacagatggaaaaacatatgatgcttttgtgtcttacctaaaagaatgccgacctgaaaatggagaggagcaca | |
| | cctttgctgtggagattttgcccagggtgttggagaaacattttgggtataagttatgcatatttgaaagggatgtagtgcct | |
| | ggaggagctgttgttgatgaaatccactcactgatagagaaaagccgaagactaatcattgtcctaagtaaaagttatat | |
| | gtctaatgaggtcaggtatgaacttgaaagtggactccatgaagcattggtggaaagaaaaattaaaataatcttaattga | |
| | atttacacctgttactgacttcacattcttgccccaatcactaaagcttttgaaatctcacagagttctgaagtggaaggccg | |
| | ataaatctctttcttataactcaaggttctggaagaaccttctttacttaatgcctgcaaaaacagtcaagccaggtagaga | |
| | cgaaccggaagtcttgcctgttctttccgagtctggaagcggtgagggaagaggtagcctgctcacgtgcggtgatgt | |
| | ggaggagaaccctgggcctatgctctgtttgggctggatatttctttggcttgttgcaggagagcgaattaaaggatttaa | |
| | tatttcaggttgttccacaaaaaaactcctttggacatattctacaaggagtgaagaggaatttgtcttattttgtgatttacca | |
| | gagccacagaaatcacatttctgccacagaaatcgactctcaccaaaacaagtccctgagcacctgcccttcatgggta | |
| | gtaacgacctatctgatgtccaatggtaccaacaaccttcgaatggagatccattagaggacattaggaaaagctatcct | |
| | cacatcattcaggacaaatgtacccttcactttttgaccccaggggtgaataattctgggtcatatatttgtagacccaaga | |
| | tgattaagagcccctatgatgtagcctgttgtgtcaagatgattttagaagttaagccccagacaaatgcatcctgtgagta | |
| | ttccgcatcacataagcaagacctacttcttgggagcactggctctatttcttgccccagtctcagctgccaaagtgatgc | |
| | acaaagtccagcggtaacctggtacaagaatggaaaactcctctctgtggaaaggagcaaccgaatcgtagtggatga | |
| | agtttatgactatcaccagggcacatatgtatgtgattacactcagtcggatactgtgagttcgtggacagtcagagctgtt | |
| | gttcaagtgagaaccattgtgggagacactaaactcaaaccagatattctggatcctgtcgaggacacactggaagtag | |
| | aacttggaaagcctttaactattagctgcaaagcacgatttggctttgaaagggtctttaaccctgtcataaaatggtacat | |
| | caaagattctgacctagagtgggaagtctcagtacctgaggcgaaaagtattaaatccactttaaaggatgaaatcattg | |
| | agcgtaatatcatcttggaaaaagtcactcagcgtgatcttcgcaggaagtttgtttgctttgtccagaactccattggaaa | |
| | cacaacccagtccgtccaactgaaagaaaagagaggagtggtgctcctgtacatcctgcttggcaccatcgggaccct | |
| | ggtggccgtgctggcggcgagtgccctcctctacaggcactggattgaaatagtgctgctgtaccggacctaccagag | |
| | caaggatcagacgcttggggataaaaaggattttgatgctttcgtatcctatgcaaaatggagctcttttccaagtgaggc | |
| | cacttcatctctgagtgaagaacacttggccctgagcctatttcctgatgttttagaaaacaaatatggatatagcctgtgtt | |
| | tgcttgaaagagatgtggctccaggaggagtgtatgcagaagacattgtgagcattattaagagaagcagaagaggaa | |
| | tatttatcttgagccccaactatgtcaatggacccagtatctttgaactacaagcagcagtgaatcttgccttggatgatca | |
| | aacactgaaactcattttaattaagttctgttacttccaagagccagagtctctacctcatctcgtgaaaaaagctctcagg | |
| | gttttgcccacagttacttggagaggcttaaaatcagttcctcccaattctaggttctgggccaaaatgcgctaccacatg | |
| | cctgtgaaaaactctcagggattcacgtggaaccagctcagaattacctctaggatttttcagtggaaaggactcagtag | |
| | aacagaaaccactgggaggagctcccagcctaaggaatggtgataa | |
| CAR 20A10N | atggccttaccagtgaccgccttgctcctgccgctggccttgctgctccacgccgccaggccgcaggtgcagctggtt | 156 |
| CD8-CD8-41BB | gaatctggcggcggacttgtgaagcctggcggatctctgagactgagctgtgccgccagcggcttcacctttagcacat | |
| CD3 | acgccatgagctggatcagacaggcccctggcaaaggcctggaatgggtggcgtctattggcagagccggcagcac | |
| 6XFOXP3NFAT | ctactacagcgactctgtgaagggcagattcaccatcagccgggacaacgccaagaacagcctgtacctgcagatga | |
| mCMV | actccctgagagccgaggacaccgccgtgtactattgtgccagaggacccatctacaacgactacgacgagttcgcct | |
| IL18Ralpha-T2A- | attggggccagggcacactggtcacagtttctagcggcggtggcggaagcggaggcggtggctccggtggcggag | |
| IL18Rb | gcagcgaaattgtgctgacacagagccccgccacactgtcactttctccaggcgaaagagccacactgagctgcaag | |
| | agcagccagagcgtgctgtactccagcaaccagaagaactacctggcctggtatcagcagaagcccggccaagctc | |
| | ctcggctgctgatctattgggccagcacaagagagagcggcatccctgccagattttctggcagcggctctggcaccg | |
| | atttcaccctgaccataagcagcctggaacctgaggacttcgccgtgtattactgccaccagtacctgagcagc ctgac | |
| | ctttggcggaggcaccaaggtggaaatcaagcggacaacaacccctgcccccagacctcctaccccagcccctaca | |
| | attgccagccagcctctgagcctgaggcccgaggcttgtagacctgctgctggcggagccgtgcacaccagaggact | |
| | ggatttcgcctgcgacatctacatctgggcgcccttggccgggacttgtggggtccttctcctgtcactggttatcaccct | |
| | ttactgcaaacggggcagaaagaaactcctgtatatattcaaacaaccatttatgagaccagtacaaactactcaagagg | |
| | aagatggctgtagctgccgatttccagaagaagaagaaggaggatgtgaactgagagtgaagttcagcaggagcgc | |
| | agacgcccccgcgtacaagcagggccagaaccagctctataacgagctcaatctaggacgaagagaggagtacgat | |
| | gttttggacaagagacgtggccgggaccctgagatggggggaaagccgagaaggaagaaccctcaggaaggcctg | |
| | tacaatgaactgcagaaagataagatggcggaggcctacagtgagattgggatgaaaggcgagcgccggaggggc | |
| | aaggggcacgatggcctttaccagggtctcagtacagccaccaaggacacctacgacgcccttcacatgcaggccct | |
| | gccccctcgctgataagtttaaactgccagaacatttctctggcctaactggccggtaccggcttcattttttccatttactg | |
| | cagaggcttcattttttccatttactgcagaggcttcattttttccatttactgcagaggcttcattttttccatttactgcagag | |
| | gcttcattttttccatttactgcagaggcttcattttttccatttactgcagaactagttaggcgtgtacggtgggaggcctat | |
| | ataagcagagctcgtttagtgaaccgtcagatcgcctggagacgccatccacgctgttttgacctccatagaagacacc | |
| | gggaccgatccagcctcgagagacccaatgctagccaccatgaattgtagagaattacccttgaccctttgggtgctta | |
| | tatctgtaagcactgcagaatcttgtacttcacgtccccacattactgtggttgaaggggaacctttctatctgaaacattgc | |
| | tcgtgttcacttgcacatgagattgaaacaaccaccaaaagctggtacaaaagcagtggatcacaggaacatgtggag | |
| | ctgaacccaaggagttccagcagaattgctttgcatgattgtgttttggagttttggccagttgagttgaatgacacaggat | |
| | cttactttttccaaatgaaaaattatactcagaaatggaaattaaatgtcatcagaagaaataaacacagctgtttcactgaa | |
| | agacaagtaactagtaaaattgtggaagttaaaaaattttttcagataacctgtgaaaacagttactatcaaacactggtca | |
| | acagcacatcattgtataagaactgtaaaaagctactactggagaacaataaaaacccaacgataaagaagaacgccg | |
| | agtttgaagatcaggggtattactcctgcgtgcatttccttcatcataatggaaaactatttaatatcaccaaaaccttcaata | |
| | taacaatagtggaagatcgcagtaatatagttccggttcttcttggaccaaagcttaaccatgttgcagtggaattaggaa | |
| | aaaacgtaaggctcaactgctctgctttgctgaatgaagaggatgtaatttattggatgttcggggaagaaaatggatcg | |
| | gatcctaatatacatgaagagaaagaaatgagaattatgactccagaaggcaaatggcatgcttcaaaagtattgagaat | |
| | tgaaaatattggtgaaagcaatctaaatgttttatataattgcactgtggccagcacgggaggcacagacaccaaaagct | |
| | tcatcttggtgagaaaagcagacatggctgatatcccaggccacgtcttcacaagaggaatgatcatagctgttttgatct | |
| | tggtggcagtagtgtgcctagtgactgtgtgtgtcatttatagagttgacttggttctattttatagacatttaacgagaaga | |
| | gatgaaacattaacagatggaaaaacatatgatgcttttgtgtcttacctaaaagaatgccgacctgaaaatggagagga | |
| | gcacacctttgctgtggagattttgcccagggtgttggagaaacattttgggtataagttatgcatatttgaaagggatgta | |
| | gtgcctggaggagctgttgttgatgaaatccactcactgatagagaaaagccgaagactaatcattgtcctaagtaaaag | |
| | ttatatgtctaatgaggtcaggtatgaacttgaaagtggactccatgaagcattggtggaaagaaaaattaaaataatctta | |
| | attgaatttacacctgttactgacttcacattcttgccccaatcactaaagcttttgaaatctcacagagttctgaagtggaa | |
| | ggccgataaatctctttcttataactcaaggttctggaagaaccttctttacttaatgcctgcaaaaacagtcaagccaggt | |
| | agagacgaaccggaagtcttgcctgttctttccgagtctggaagcggtgagggaagaggtagcctgctcacgtgcggt | |
| | gatgtggaggagaaccctgggcctatgctctgtttgggctggatatttctttggcttgttgcaggagagcgaattaaagg | |
| | atttaatatttcaggttgttccacaaaaaaactcctttggacatattctacaaggagtgaagaggaatttgtcttattttgtgat | |
| | ttaccagagccacagaaatcacatttctgccacagaaatcgactctcaccaaaacaagtccctgagcacctgcccttcat | |
| | gggtagtaacgacctatctgatgtccaatggtaccaacaaccttcgaatggagatccattagaggacattaggaaaagc | |
| | tatcctcacatcattcaggacaaatgtacccttcactttttgaccccaggggtgaataattctgggtcatatatttgtagacc | |
| | caagatgattaagagcccctatgatgtagcctgttgtgtcaagatgattttagaagttaagccccagacaaatgcatcctg | |
| | tgagtattccgcatcacataagcaagacctacttcttgggagcactggctctatttcttgccccagtctcagctgccaaagt | |
| | gatgcacaaagtccagcggtaacctggtacaagaatggaaaactcctctctgtggaaaggagcaaccgaatcgtagtg | |
| | gatgaagtttatgactatcaccagggcacatatgtatgtgattacactcagtcggatactgtgagttcgtggacagtcaga | |
| | gctgttgttcaagtgagaaccattgtgggagacactaaactcaaaccagatattctggatcctgtcgaggacacactgga | |
| | agtagaacttggaaagcctttaactattagctgcaaagcacgatttggctttgaaagggtctttaaccctgtcataaaatgg | |
| | tacatcaaagattctgacctagagtgggaagtctcagtacctgaggcgaaaagtattaaatccactttaaaggatgaaat | |
| | cattgagcgtaatatcatcttggaaaaagtcactcagcgtgatcttcgcaggaagtttgtttgctttgtccagaactccattg | |
| | gaaacacaacccagtccgtccaactgaaagaaaagagaggagtggtgctcctgtacatcctgcttggcaccatcggg | |
| | accctggtggccgtgctggcggcgagtgccctcctctacaggcactggattgaaatagtgctgctgtaccggacctac | |
| | cagagcaaggatcagacgcttggggataaaaaggattttgatgctttcgtatcctatgcaaaatggagctcttttccaagt | |
| | gaggccacttcatctctgagtgaagaacacttggccctgagcctatttcctgatgttttagaaaacaaatatggatatagc | |
| | ctgtgtttgcttgaaagagatgtggctccaggaggagtgtatgcagaagacattgtgagcattattaagagaagcagaa | |
| | gaggaatatttatcttgagccccaactatgtcaatggacccagtatctttgaactacaagcagcagtgaatcttgccttgg | |
| | atgatcaaacactgaaactcattttaattaagttctgttacttccaagagccagagtctctacctcatctcgtgaaaaaagct | |
| | ctcagggttttgcccacagttacttggagaggcttaaaatcagttcctcccaattctaggttctgggccaaaatgcgctac | |
| | cacatgcctgtgaaaaactctcagggattcacgtggaaccagctcagaattacctctaggatttttcagtggaaaggact | |
| | cagtagaacagaaaccactgggaggagctcccagcctaaggaatggtgataa | |
| CAR 20A10N | atggccttaccagtgaccgccttgctcctgccgctggccttgctgctccacgccgccaggccgcaggtgcagctggtt | 157 |
| CD8-CD8-41BB | gaatctggcggcggacttgtgaagcctggcggatctctgagactgagctgtgccgccagcggcttcacctttagcacat | |
| CD3 | acgccatgagctggatcagacaggcccctggcaaaggcctggaatgggtggcgtctattggcagagccggcagcac | |
| 3XFOXP3NFAT | ctactacagcgactctgtgaagggcagattcaccatcagccgggacaacgccaagaacagcctgtacctgcagatga | |
| mCMV | actccctgagagccgaggacaccgccgtgtactattgtgccagaggacccatctacaacgactacgacgagttcgcct | |
| IL18Ralpha-T2A- | attggggccagggcacactggtcacagtttctagcggcggtggcggaagcggaggcggtggctccggtggcggag | |
| IL18Rb | gcagcgaaattgtgctgacacagagccccgccacactgtcactttctccaggcgaaagagccacactgagctgcaag | |
| | agcagccagagcgtgctgtactccagcaaccagaagaactacctggcctggtatcagcagaagcccggccaagctc | |
| | ctcggctgctgatctattgggccagcacaagagagagcggcatccctgccagattttctggcagcggctctggcaccg | |
| | atttcaccctgaccataagcagcctggaacctgaggacttcgccgtgtattactgccaccagtacctgagcagcctgac | |
| | ctttggcggaggcaccaaggtggaaatcaagcggacaacaacccctgcccccagacctcctaccccagcccctaca | |
| | attgccagccagcctctgagcctgaggcccgaggcttgtagacctgctgctggcggagccgtgcacaccagaggact | |
| | ggatttcgcctgcgacatctacatctgggcgcccttggccgggacttgtggggtccttctcctgtcactggttatcaccct | |
| | ttactgcaaacggggcagaaagaaactcctgtatatattcaaacaaccatttatgagaccagtacaaactactcaagagg | |
| | aagatggctgtagctgccgatttccagaagaagaagaaggaggatgtgaactgagagtgaagttcagcaggagcgc | |
| | agacgcccccgcgtacaagcagggccagaaccagctctataacgagctcaatctaggacgaagagaggagtacgat | |
| | gttttggacaagagacgtggccgggaccctgagatggggggaaagccgagaaggaagaaccctcaggaaggcctg | |
| | tacaatgaactgcagaaagataagatggcggaggcctacagtgagattgggatgaaaggcgagcgccggaggggc | |
| | aaggggcacgatggcctttaccagggtctcagtacagccaccaaggacacctacgacgcccttcacatgcaggccct | |
| | gccccctcgctgataagtttaaactgccagaacatttctctggcctaactggccggtaccggaggaaaaactgtttcata | |
| | cagaaggcgtggaggaaaaactgtttcatacagaaggcgtggaggaaaaactgtttcatacagaaggcgtggaggaa | |
| | aaactgtttcatacagaaggcgtggaggaaaaactgtttcatacagaaggcgtggaggaaaaactgtttcatacagaag | |
| | gcgtactagttaggcgtgtacggtgggaggcctatataagcagagctcgtttagtgaaccgtcagatcgcctggagac | |
| | gccatccacgctgttttgacctccatagaagacaccgggaccgatccagcctcgagagacccaatgctagccaccat | |
| | gaattgtagagaattacccttgaccctttgggtgcttatatctgtaagcactgcagaatcttgtacttcacgtccccacatta | |
| | ctgtggttgaaggggaacctttctatctgaaacattgctcgtgttcacttgcacatgagattgaaacaaccaccaaaagct | |
| | ggtacaaaagcagtggatcacaggaacatgtggagctgaacccaaggagttccagcagaattgctttgcatgattgtgt | |
| | tttggagttttggccagttgagttgaatgacacaggatcttactttttccaaatgaaaaattatactcagaaatggaaattaa | |
| | atgtcatcagaagaaataaacacagctgtttcactgaaagacaagtaactagtaaaattgtggaagttaaaaaattttttca | |
| | gataacctgtgaaaacagttactatcaaacactggtcaacagcacatcattgtataagaactgtaaaaagctactactgg | |
| | agaacaataaaaacccaacgataaagaagaacgccgagtttgaagatcaggggtattactcctgcgtgcatttccttcat | |
| | cataatggaaaactatttaatatcaccaaaaccttcaatataacaatagtggaagatcgcagtaatatagttccggttcttct | |
| | tggaccaaagcttaaccatgttgcagtggaattaggaaaaaacgtaaggctcaactgctctgctttgctgaatgaagag | |
| | gatgtaatttattggatgttcggggaagaaaatggatcggatcctaatatacatgaagagaaagaaatgagaattatgact | |
| | ccagaaggcaaatggcatgcttcaaaagtattgagaattgaaaatattggtgaaagcaatctaaatgttttatataattgca | |
| | ctgtggccagcacgggaggcacagacaccaaaagcttcatcttggtgagaaaagcagacatggctgatatcccaggc | |
| | cacgtcttcacaagaggaatgatcatagctgttttgatcttggtggcagtagtgtgcctagtgactgtgtgtgtcatttatag | |
| | agttgacttggttctattttatagacatttaacgagaagagatgaaacattaacagatggaaaaacatatgatgcttttgtgt | |
| | cttacctaaaagaatgccgacctgaaaatggagaggagcacacctttgctgtggagattttgcccagggtgttggagaa | |
| | acattttgggtataagttatgcatatttgaaagggatgtagtgcctggaggagctgttgttgatgaaatccactcactgata | |
| | gagaaaagccgaagactaatcattgtcctaagtaaaagttatatgtctaatgaggtcaggtatgaacttgaaagtggact | |
| | ccatgaagcattggtggaaagaaaaattaaaataatcttaattgaatttacacctgttactgacttcacattcttgccccaat | |
| | cactaaagcttttgaaatctcacagagttctgaagtggaaggccgataaatctctttcttataactcaaggttctggaagaa | |
| | ccttctttacttaatgcctgcaaaaacagtcaagccaggtagagacgaaccggaagtcttgcctgttctttccgagtctgg | |
| | aagcggtgagggaagaggtagcctgctcacgtgcggtgatgtggaggagaaccctgggcctatgctctgtttgggct | |
| | ggatatttctttggcttgttgcaggagagcgaattaaaggatttaatatttcaggttgttccacaaaaaaactcctttggacat | |
| | attctacaaggagtgaagaggaatttgtcttattttgtgatttaccagagccacagaaatcacatttctgccacagaaatcg | |
| | actctcaccaaaacaagtccctgagcacctgcccttcatgggtagtaacgacctatctgatgtccaatggtaccaacaac | |
| | cttcgaatggagatccattagaggacattaggaaaagctatcctcacatcattcaggacaaatgtacccttcactttttgac | |
| | cccaggggtgaataattctgggtcatatatttgtagacccaagatgattaagagcccctatgatgtagcctgttgtgtcaa | |
| | gatgattttagaagttaagccccagacaaatgcatcctgtgagtattccgcatcacataagcaagacctacttcttgggag | |
| | cactggctctatttcttgccccagtctcagctgccaaagtgatgcacaaagtccagcggtaacctggtacaagaatgga | |
| | aaactcctctctgtggaaaggagcaaccgaatcgtagtggatgaagtttatgactatcaccagggcacatatgtatgtga | |
| | ttacactcagtcggatactgtgagttcgtggacagtcagagctgttgttcaagtgagaaccattgtgggagacactaaac | |
| | tcaaaccagatattctggatcctgtcgaggacacactggaagtagaacttggaaagcctttaactattagctgcaaagca | |
| | cgatttggctttgaaagggtctttaaccctgtcataaaatggtacatcaaagattctgacctagagtgggaagtctcagtac | |
| | ctgaggcgaaaagtattaaatccactttaaaggatgaaatcattgagcgtaatatcatcttggaaaaagtcactcagcgtg | |
| | atcttcgcaggaagtttgtttgctttgtccagaactccattggaaacacaacccagtccgtccaactgaaagaaaagaga | |
| | ggagtggtgctcctgtacatcctgcttggcaccatcgggaccctggtggccgtgctggcggcgagtgccctcctctac | |
| | aggcactggattgaaatagtgctgctgtaccggacctaccagagcaaggatcagacgcttggggataaaaaggatttt | |
| | gatgctttcgtatcctatgcaaaatggagctcttttccaagtgaggccacttcatctctgagtgaagaacacttggccctga | |
| | gcctatttcctgatgttttagaaaacaaatatggatatagcctgtgtttgcttgaaagagatgtggctccaggaggagtgta | |
| | tgcagaagacattgtgagcattattaagagaagcagaagaggaatatttatcttgagccccaactatgtcaatggaccca | |
| | gtatctttgaactacaagcagcagtgaatcttgccttggatgatcaaacactgaaactcattttaattaagttctgttacttcc | |
| | aagagccagagtctctacctcatctcgtgaaaaaagctctcagggttttgcccacagttacttggagaggcttaaaatca | |
| | gttcctcccaattctaggttctgggccaaaatgcgctaccacatgcctgtgaaaaactctcagggattcacgtggaacca | |
| | gctcagaattacctctaggatttttcagtggaaaggactcagtagaacagaaaccactgggaggagctcccagcctaa | |
| | ggaatggtgataa | |
| CAR 20A10N | atggccttaccagtgaccgccttgctcctgccgctggccttgctgctccacgccgccaggccgcaggtgcagctggtt | 158 |
| CD8-CD8-41BB | gaatctggcggcggacttgtgaagcctggcggatctctgagactgagctgtgccgccagcggcttcacctttagcacat | |
| CD3 6XIL2NFAT | acgccatgagctggatcagacaggcccctggcaaaggcctggaatgggtggcgtctattggcagagccggcagcac | |
| mCMV | ctactacagcgactctgtgaagggcagattcaccatcagccgggacaacgccaagaacagcctgtacctgcagatga | |
| IL18Ralpha-T2A- | actccctgagagccgaggacaccgccgtgtactattgtgccagaggacccatctacaacgactacgacgagttcgcct | |
| IL18Rb | attggggccagggcacactggtcacagtttctagcggcggtggcggaagcggaggcggtggctccggtggcggag | |
| | gcagcgaaattgtgctgacacagagccccgccacactgtcactttctccaggcgaaagagccacactgagctgcaag | |
| | agcagccagagcgtgctgtactccagcaaccagaagaactacctggcctggtatcagcagaagcccggccaagctc | |
| | ctcggctgctgatctattgggccagcacaagagagagcggcatccctgccagattttctggcagcggctctggcaccg | |
| | atttcaccctgaccataagcagcctggaacctgaggacttcgccgtgtattactgccaccagtacctgagcagcctgac | |
| | ctttggcggaggcaccaaggtggaaatcaagcggacaacaacccctgcccccagacctcctaccccagcccctaca | |
| | attgccagccagcctctgagcctgaggcccgaggcttgtagacctgctgctggcggagccgtgcacaccagaggact | |
| | ggatttcgcctgcgacatctacatctgggcgcccttggccgggacttgtggggtccttctcctgtcactggttatcaccct | |
| | ttactgcaaacggggcagaaagaaactcctgtatatattcaaacaaccatttatgagaccagtacaaactactcaagagg | |
| | aagatggctgtagctgccgatttccagaagaagaagaaggaggatgtgaactgagagtgaagttcagcaggagcgc | |
| | agacgcccccgcgtacaagcagggccagaaccagctctataacgagctcaatctaggacgaagagaggagtacgat | |
| | gttttggacaagagacgtggccgggaccctgagatggggggaaagccgagaaggaagaaccctcaggaaggcctg | |
| | tacaatgaactgcagaaagataagatggcggaggcctacagtgagattgggatgaaaggcgagcgccggaggggc | |
| | aaggggcacgatggcctttaccagggtctcagtacagccaccaaggacacctacgacgcccttcacatgcaggccct | |
| | gccccctcgctgataagtttaaactgccagaacatttctctggcctaactggccggtaccggaggaaaaactgtttcata | |
| | cagaaggcgtggaggaaaaactgtttcatacagaaggcgtggaggaaaaactgtttcatacagaaggcgtggaggaa | |
| | aaactgtttcatacagaaggcgtggaggaaaaactgtttcatacagaaggcgtggaggaaaaactgtttcatacagaag | |
| | gcgtactagttaggcgtgtacggtgggaggcctatataagcagagctcgtttagtgaaccgtcagatcgcctggagac | |
| | gccatccacgctgttttgacctccatagaagacaccgggaccgatccagcctcgagagacccaatgctagccaccat | |
| | gaattgtagagaattacccttgaccctttgggtgcttatatctgtaagcactgcagaatcttgtacttcacgtccccacatta | |
| | ctgtggttgaaggggaacctttctatctgaaacattgctcgtgttcacttgcacatgagattgaaacaaccaccaaaagct | |
| | ggtacaaaagcagtggatcacaggaacatgtggagctgaacccaaggagttccagcagaattgctttgcatgattgtgt | |
| | tttggagttttggccagttgagttgaatgacacaggatcttactttttccaaatgaaaaattatactcagaaatggaaattaa | |
| | atgtcatcagaagaaataaacacagctgtttcactgaaagacaagtaactagtaaaattgtggaagttaaaaaattttttca | |
| | gataacctgtgaaaacagttactatcaaacactggtcaacagcacatcattgtataagaactgtaaaaagctactactgg | |
| | agaacaataaaaacccaacgataaagaagaacgccgagtttgaagatcaggggtattactcctgcgtgcatttccttcat | |
| | cataatggaaaactatttaatatcaccaaaaccttcaatataacaatagtggaagatcgcagtaatatagttccggttcttct | |
| | tggaccaaagcttaaccatgttgcagtggaattaggaaaaaacgtaaggctcaactgctctgctttgctgaatgaagag | |
| | gatgtaatttattggatgttcggggaagaaaatggatcggatcctaatatacatgaagagaaagaaatgagaattatgact | |
| | ccagaaggcaaatggcatgcttcaaaagtattgagaattgaaaatattggtgaaagcaatctaaatgttttatataattgca | |
| | ctgtggccagcacgggaggcacagacaccaaaagcttcatcttggtgagaaaagcagacatggctgatatcccaggc | |
| | cacgtcttcacaagaggaatgatcatagctgttttgatcttggtggcagtagtgtgcctagtgactgtgtgtgtcatttatag | |
| | agttgacttggttctattttatagacatttaacgagaagagatgaaacattaacagatggaaaaacatatgatgcttttgtgt | |
| | cttacctaaaagaatgccgacctgaaaatggagaggagcacacctttgctgtggagattttgcccagggtgttggagaa | |
| | acattttgggtataagttatgcatatttgaaagggatgtagtgcctggaggagctgttgttgatgaaatccactcactgata | |
| | gagaaaagccgaagactaatcattgtcctaagtaaaagttatatgtctaatgaggtcaggtatgaacttgaaagtggact | |
| | ccatgaagcattggtggaaagaaaaattaaaataatcttaattgaatttacacctgttactgacttcacattcttgccccaat | |
| | cactaaagcttttgaaatctcacagagttctgaagtggaaggccgataaatctctttcttataactcaaggttctggaagaa | |
| | ccttctttacttaatgcctgcaaaaacagtcaagccaggtagagacgaaccggaagtcttgcctgtttttccgagtctgg | |
| | aagcggtgagggaagaggtagcctgctcacgtgcggtgatgtggaggagaaccctgggcctatgctctgtttgggct | |
| | ggatatttctttggcttgttgcaggagagcgaattaaaggatttaatatttcaggttgttccacaaaaaaactcctttggacat | |
| | attctacaaggagtgaagaggaatttgtcttattttgtgatttaccagagccacagaaatcacatttctgccacagaaatcg | |
| | actctcaccaaaacaagtccctgagcacctgcccttcatgggtagtaacgacctatctgatgtccaatggtaccaacaac | |
| | cttcgaatggagatccattagaggacattaggaaaagctatcctcacatcattcaggacaaatgtacccttcactttttgac | |
| | cccaggggtgaataattctgggtcatatatttgtagacccaagatgattaagagcccctatgatgtagcctgttgtgtcaa | |
| | gatgattttagaagttaagccccagacaaatgcatcctgtgagtattccgcatcacataagcaagacctacttcttgggag | |
| | cactggctctatttcttgccccagtctcagctgccaaagtgatgcacaaagtccagcggtaacctggtacaagaatgga | |
| | aaactcctctctgtggaaaggagcaaccgaatcgtagtggatgaagtttatgactatcaccagggcacatatgtatgtga | |
| | ttacactcagtcggatactgtgagttcgtggacagtcagagctgttgttcaagtgagaaccattgtgggagacactaaac | |
| | tcaaaccagatattctggatcctgtcgaggacacactggaagtagaacttggaaagcctttaactattagctgcaaagca | |
| | cgatttggctttgaaagggtctttaaccctgtcataaaatggtacatcaaagattctgacctagagtgggaagtctcagtac | |
| | ctgaggcgaaaagtattaaatccactttaaaggatgaaatcattgagcgtaatatcatcttggaaaaagtcactcagcgtg | |
| | atcttcgcaggaagtttgtttgctttgtccagaactccattggaaacacaacccagtccgtccaactgaaagaaaagaga | |
| | ggagtggtgctcctgtacatcctgcttggcaccatcgggaccctggtggccgtgctggcggcgagtgccctcctctac | |
| | aggcactggattgaaatagtgctgctgtaccggacctaccagagcaaggatcagacgcttggggataaaaaggatttt | |
| | gatgctttcgtatcctatgcaaaatggagctcttttccaagtgaggccacttcatctctgagtgaagaacacttggccctga | |
| | gcctatttcctgatgttttagaaaacaaatatggatatagcctgtgtttgcttgaaagagatgtggctccaggaggagtgta | |
| | tgcagaagacattgtgagcattattaagagaagcagaagaggaatatttatcttgagccccaactatgtcaatggaccca | |
| | gtatctttgaactacaagcagcagtgaatcttgccttggatgatcaaacactgaaactcattttaattaagttctgttacttcc | |
| | aagagccagagtctctacctcatctcgtgaaaaaagctctcagggttttgcccacagttacttggagaggcttaaaatca | |
| | gttcctcccaattctaggttctgggccaaaatgcgctaccacatgcctgtgaaaaactctcagggattcacgtggaacca | |
| | gctcagaattacctctaggatttttcagtggaaaggactcagtagaacagaaaccactgggaggagctcccagcctaa | |
| | ggaatggtgataa | |
| CAR 20A10N | atggccttaccagtgaccgccttgctcctgccgctggccttgctgctccacgccgccaggccgcaggtgcagctggtt | 159 |
| CD8-CD8-41BB | gaatctggcggcggacttgtgaagcctggcggatctctgagactgagctgtgccgccagcggcttcacctttagcacat | |
| CD3 3XIL2NFAT | acgccatgagctggatcagacaggcccctggcaaaggcctggaatgggtggcgtctattggcagagccggcagcac | |
| mCMV | ctactacagcgactctgtgaagggcagattcaccatcagccgggacaacgccaagaacagcctgtacctgcagatga | |
| IL18Ralpha-T2A- | actccctgagagccgaggacaccgccgtgtactattgtgccagaggacccatctacaacgactacgacgagttcgcct | |
| IL18Rb | attggggccagggcacactggtcacagtttctagcggcggtggcggaagcggaggcggtggctccggtggcggag | |
| | gcagcgaaattgtgctgacacagagccccgccacactgtcactttctccaggcgaaagagccacactgagctgcaag | |
| | agcagccagagcgtgctgtactccagcaaccagaagaactacctggcctggtatcagcagaagcccggccaagctc | |
| | ctcggctgctgatctattgggccagcacaagagagagcggcatccctgccagattttctggcagcggctctggcaccg | |
| | atttcaccctgaccataagcagcctggaacctgaggacttcgccgtgtattactgccaccagtacctgagcagcctgac | |
| | ctttggcggaggcaccaaggtggaaatcaagcggacaacaacccctgcccccagacctcctaccccagcccctaca | |
| | attgccagccagcctctgagcctgaggcccgaggcttgtagacctgctgctggcggagccgtgcacaccagaggact | |
| | ggatttcgcctgcgacatctacatctgggcgcccttggccgggacttgtggggtccttctcctgtcactggttatcaccct | |
| | ttactgcaaacggggcagaaagaaactcctgtatatattcaaacaaccatttatgagaccagtacaaactactcaagagg | |
| | aagatggctgtagctgccgatttccagaagaagaagaaggaggatgtgaactgagagtgaagttcagcaggagcgc | |
| | agacgcccccgcgtacaagcagggccagaaccagctctataacgagctcaatctaggacgaagagaggagtacgat | |
| | gttttggacaagagacgtggccgggaccctgagatggggggaaagccgagaaggaagaaccctcaggaaggcctg | |
| | tacaatgaactgcagaaagataagatggcggaggcctacagtgagattgggatgaaaggcgagcgccggaggggc | |
| | aaggggcacgatggcctttaccagggtctcagtacagccaccaaggacacctacgacgcccttcacatgcaggccct | |
| | gccccctcgctgataagtttaaactgccagaacatttctctggcctaactggccggtaccggaggaaaaactgtttcata | |
| | cagaaggcgtggaggaaaaactgtttcatacagaaggcgtggaggaaaaactgtttcatacagaaggcgtactagtta | |
| | ggcgtgtacggtgggaggcctatataagcagagctcgtttagtgaaccgtcagatcgcctggagacgccatccacgct | |
| | gttttgacctccatagaagacaccgggaccgatccagcctcgagagacccaatgctagccaccatgaattgtagaga | |
| | attacccttgaccctttgggtgcttatatctgtaagcactgcagaatcttgtacttcacgtccccacattactgtggttgaag | |
| | gggaacctttctatctgaaacattgctcgtgttcacttgcacatgagattgaaacaaccaccaaaagctggtacaaaagc | |
| | agtggatcacaggaacatgtggagctgaacccaaggagttccagcagaattgctttgcatgattgtgttttggagttttgg | |
| | ccagttgagttgaatgacacaggatcttactttttccaaatgaaaaattatactcagaaatggaaattaaatgtcatcagaa | |
| | gaaataaacacagctgtttcactgaaagacaagtaactagtaaaattgtggaagttaaaaaattttttcagataacctgtga | |
| | aaacagttactatcaaacactggtcaacagcacatcattgtataagaactgtaaaaagctactactggagaacaataaaa | |
| | acccaacgataaagaagaacgccgagtttgaagatcaggggtattactcctgcgtgcatttccttcatcataatggaaaa | |
| | ctatttaatatcaccaaaaccttcaatataacaatagtggaagatcgcagtaatatagttccggttcttcttggaccaaagct | |
| | taaccatgttgcagtggaattaggaaaaaacgtaaggctcaactgctctgctttgctgaatgaagaggatgtaatttattg | |
| | gatgttcggggaagaaaatggatcggatcctaatatacatgaagagaaagaaatgagaattatgactccagaaggcaa | |
| | atggcatgcttcaaaagtattgagaattgaaaatattggtgaaagcaatctaaatgttttatataattgcactgtggccagca | |
| | cgggaggcacagacaccaaaagcttcatcttggtgagaaaagcagacatggctgatatcccaggccacgtcttcaca | |
| | agaggaatgatcatagctgttttgatcttggtggcagtagtgtgcctagtgactgtgtgtgtcatttatagagttgacttggtt | |
| | ctattttatagacatttaacgagaagagatgaaacattaacagatggaaaaacatatgatgcttttgtgtcttacctaaaaga | |
| | atgccgacctgaaaatggagaggagcacacctttgctgtggagattttgcccagggtgttggagaaacattttgggtata | |
| | agttatgcatatttgaaagggatgtagtgcctggaggagctgttgttgatgaaatccactcactgatagagaaaagccga | |
| | agactaatcattgtcctaagtaaaagttatatgtctaatgaggtcaggtatgaacttgaaagtggactccatgaagcattgg | |
| | tggaaagaaaaattaaaataatcttaattgaatttacacctgttactgacttcacattcttgccccaatcactaaagcttttga | |
| | aatctcacagagttctgaagtggaaggccgataaatctctttcttataactcaaggttctggaagaaccttctttacttaatg | |
| | cctgcaaaaacagtcaagccaggtagagacgaaccggaagtcttgcctgttctttccgagtctggaagcggtgaggga | |
| | agaggtagcctgctcacgtgcggtgatgtggaggagaaccctgggcctatgctctgtttgggctggatatttctttggctt | |
| | gttgcaggagagcgaattaaaggatttaatatttcaggttgttccacaaaaaaactcctttggacatattctacaaggagtg | |
| | aagaggaatttgtcttattttgtgatttaccagagccacagaaatcacatttctgccacagaaatcgactctcaccaaaaca | |
| | agtccctgagcacctgcccttcatgggtagtaacgacctatctgatgtccaatggtaccaacaaccttcgaatggagatc | |
| | cattagaggacattaggaaaagctatcctcacatcattcaggacaaatgtacccttcactttttgaccccaggggtgaata | |
| | attctgggtcatatatttgtagacccaagatgattaagagcccctatgatgtagcctgttgtgtcaagatgattttagaagtt | |
| | aagccccagacaaatgcatcctgtgagtattccgcatcacataagcaagacctacttcttgggagcactggctctatttct | |
| | tgccccagtctcagctgccaaagtgatgcacaaagtccagcggtaacctggtacaagaatggaaaactcctctctgtgg | |
| | aaaggagcaaccgaatcgtagtggatgaagtttatgactatcaccagggcacatatgtatgtgattacactcagtcggat | |
| | actgtgagttcgtggacagtcagagctgttgttcaagtgagaaccattgtgggagacactaaactcaaaccagatattct | |
| | ggatcctgtcgaggacacactggaagtagaacttggaaagcctttaactattagctgcaaagcacgatttggctttgaaa | |
| | gggtctttaaccctgtcataaaatggtacatcaaagattctgacctagagtgggaagtctcagtacctgaggcgaaaagt | |
| | attaaatccactttaaaggatgaaatcattgagcgtaatatcatcttggaaaaagtcactcagcgtgatcttcgcaggaagt | |
| | ttgtttgctttgtccagaactccattggaaacacaacccagtccgtccaactgaaagaaaagagaggagtggtgctcctg | |
| | tacatcctgcttggcaccatcgggaccctggtggccgtgctggcggcgagtgccctcctctacaggcactggattgaa | |
| | atagtgctgctgtaccggacctaccagagcaaggatcagacgcttggggataaaaaggattttgatgctttcgtatcctat | |
| | gcaaaatggagctcttttccaagtgaggccacttcatctctgagtgaagaacacttggccctgagcctatttcctgatgttt | |
| | tagaaaacaaatatggatatagcctgtgtttgcttgaaagagatgtggctccaggaggagtgtatgcagaagacattgtg | |
| | agcattattaagagaagcagaagaggaatatttatcttgagccccaactatgtcaatggacccagtatctttgaactacaa | |
| | gcagcagtgaatcttgccttggatgatcaaacactgaaactcattttaattaagttctgttacttccaagagccagagtctct | |
| | acctcatctcgtgaaaaaagctctcagggttttgcccacagttacttggagaggcttaaaatcagttcctcccaattctagg | |
| | ttctgggccaaaatgcgctaccacatgcctgtgaaaaactctcagggattcacgtggaaccagctcagaattacctctag | |
| | gatttttcagtggaaaggactcagtagaacagaaaccactgggaggagctcccagcctaaggaatggtgataa | |
| CAR 20A10N | atggccttaccagtgaccgccttgctcctgccgctggccttgctgctccacgccgccaggccgcaggtgcagctggtt | 160 |
| CD28-CD28-CD28 | gaatctggcggcggacttgtgaagcctggcggatctctgagactgagctgtgccgccagcggcttcacctttagcacat | |
| CD3 | acgccatgagctggatcagacaggcccctggcaaaggcctggaatgggtggcgtctattggcagagccggcagcac | |
| 6XFOXP3NFAT | ctactacagcgactctgtgaagggcagattcaccatcagccgggacaacgccaagaacagcctgtacctgcagatga | |
| mCMV | actccctgagagccgaggacaccgccgtgtactattgtgccagaggacccatctacaacgactacgacgagttcgcct | |
| IL18Ralpha-T2A- | attggggccagggcacactggtcacagtttctagcggcggtggcggaagcggaggcggtggctccggtggcggag | |
| IL18Rb | gcagcgaaattgtgctgacacagagccccgccacactgtcactttctccaggcgaaagagccacactgagctgcaag | |
| | agcagccagagcgtgctgtactccagcaaccagaagaactacctggcctggtatcagcagaagcccggccaagctc | |
| | ctcggctgctgatctattgggccagcacaagagagagcggcatccctgccagattttctggcagcggctctggcaccg | |
| | atttcaccctgaccataagcagcctggaacctgaggacttcgccgtgtattactgccaccagtacctgagcagcctgac | |
| | ctttggcggaggcaccaaggtggaaatcaagcggaagcacctgtgtccttctccactgttccccggccctagcaagcc | |
| | tttctgggtcctggtggtggtgggcggagtgctggcctgctacagcctgctggtgaccgtggcctttatcatcttctgggt | |
| | gaggagtaagaggagcaggctcctgcacagtgactacatgaacatgactcctagaagacctgggcctaccagaaag | |
| | cattaccagccctatgccccaccacgcgacttcgcagcctatcgctccagagtgaagttcagcaggagcgcagacgc | |
| | ccccgcgtacaagcagggccagaaccagctctataacgagctcaatctaggacgaagagaggagtacgatgttttgg | |
| | acaagagacgtggccgggaccctgagatggggggaaagccgagaaggaagaaccctcaggaaggcctgtacaat | |
| | gaactgcagaaagataagatggcggaggcctacagtgagattgggatgaaaggcgagcgccggaggggcaaggg | |
| | gcacgatggcctttaccagggtctcagtacagccaccaaggacacctacgacgcccttcacatgcaggccctgcccc | |
| | ctcgctgataagtttaaactgccagaacatttctctggcctaactggccggtaccggcttcattttttccatttactgcagag | |
| | gcttcattttttccatttactgcagaggcttcattttttccatttactgcagaggcttcattttttccatttactgcagaggcttcat | |
| | tttttccatttactgcagaggcttcattttttccatttactgcagaactagttaggcgtgtacggtgggaggcctatataagca | |
| | gagctcgtttagtgaaccgtcagatcgcctggagacgccatccacgctgttttgacctccatagaagacaccgggacc | |
| | gatccagcctcgagagacccaatgctagccaccatgaattgtagagaattacccttgaccctttgggtgcttatatctgt | |
| | aagcactgcagaatcttgtacttcacgtccccacattactgtggttgaaggggaacctttctatctgaaacattgctcgtgtt | |
| | cacttgcacatgagattgaaacaaccaccaaaagctggtacaaaagcagtggatcacaggaacatgtggagctgaac | |
| | ccaaggagttccagcagaattgctttgcatgattgtgttttggagttttggccagttgagttgaatgacacaggatcttactt | |
| | tttccaaatgaaaaattatactcagaaatggaaattaaatgtcatcagaagaaataaacacagctgtttcactgaaagaca | |
| | agtaactagtaaaattgtggaagttaaaaaattttttcagataacctgtgaaaacagttactatcaaacactggtcaacagc | |
| | acatcattgtataagaactgtaaaaagctactactggagaacaataaaaacccaacgataaagaagaacgccgagtttg | |
| | aagatcaggggtattactcctgcgtgcatttccttcatcataatggaaaactatttaatatcaccaaaaccttcaatataaca | |
| | atagtggaagatcgcagtaatatagttccggttcttcttggaccaaagcttaaccatgttgcagtggaattaggaaaaaac | |
| | gtaaggctcaactgctctgctttgctgaatgaagaggatgtaatttattggatgttcggggaagaaaatggatcggatcct | |
| | aatatacatgaagagaaagaaatgagaattatgactccagaaggcaaatggcatgcttcaaaagtattgagaattgaaa | |
| | atattggtgaaagcaatctaaatgttttatataattgcactgtggccagcacgggaggcacagacaccaaaagcttcatct | |
| | tggtgagaaaagcagacatggctgatatcccaggccacgtcttcacaagaggaatgatcatagctgttttgatcttggtg | |
| | gcagtagtgtgcctagtgactgtgtgtgtcatttatagagttgacttggttctattttatagacatttaacgagaagagatga | |
| | aacattaacagatggaaaaacatatgatgcttttgtgtcttacctaaaagaatgccgacctgaaaatggagaggagcaca | |
| | cctttgctgtggagattttgcccagggtgttggagaaacattttgggtataagttatgcatatttgaaagggatgtagtgcct | |
| | ggaggagctgttgttgatgaaatccactcactgatagagaaaagccgaagactaatcattgtcctaagtaaaagttatat | |
| | gtctaatgaggtcaggtatgaacttgaaagtggactccatgaagcattggtggaaagaaaaattaaaataatcttaattga | |
| | atttacacctgttactgacttcacattcttgccccaatcactaaagcttttgaaatctcacagagttctgaagtggaaggccg | |
| | ataaatctctttcttataactcaaggttctggaagaaccttctttacttaatgcctgcaaaaacagtcaagccaggtagaga | |
| | cgaaccggaagtcttgcctgttctttccgagtctggaagcggtgagggaagaggtagcctgctcacgtgcggtgatgt | |
| | ggaggagaaccctgggcctatgctctgtttgggctggatatttctttggcttgttgcaggagagcgaattaaaggatttaa | |
| | tatttcaggttgttccacaaaaaaactcctttggacatattctacaaggagtgaagaggaatttgtcttattttgtgatttacca | |
| | gagccacagaaatcacatttctgccacagaaatcgactctcaccaaaacaagtccctgagcacctgcccttcatgggta | |
| | gtaacgacctatctgatgtccaatggtaccaacaaccttcgaatggagatccattagaggacattaggaaaagctatcct | |
| | cacatcattcaggacaaatgtacccttcactttttgaccccaggggtgaataattctgggtcatatatttgtagacccaaga | |
| | tgattaagagcccctatgatgtagcctgttgtgtcaagatgattttagaagttaagccccagacaaatgcatcctgtgagta | |
| | ttccgcatcacataagcaagacctacttcttgggagcactggctctatttcttgccccagtctcagctgccaaagtgatgc | |
| | acaaagtccagcggtaacctggtacaagaatggaaaactcctctctgtggaaaggagcaaccgaatcgtagtggatga | |
| | agtttatgactatcaccagggcacatatgtatgtgattacactcagtcggatactgtgagttcgtggacagtcagagctgtt | |
| | gttcaagtgagaaccattgtgggagacactaaactcaaaccagatattctggatcctgtcgaggacacactggaagtag | |
| | aacttggaaagcctttaactattagctgcaaagcacgatttggctttgaaagggtctttaaccctgtcataaaatggtacat | |
| | caaagattctgacctagagtgggaagtctcagtacctgaggcgaaaagtattaaatccactttaaaggatgaaatcattg | |
| | agcgtaatatcatcttggaaaaagtcactcagcgtgatcttcgcaggaagtttgtttgctttgtccagaactccattggaaa | |
| | cacaacccagtccgtccaactgaaagaaaagagaggagtggtgctcctgtacatcctgcttggcaccatcgggaccct | |
| | ggtggccgtgctggcggcgagtgccctcctctacaggcactggattgaaatagtgctgctgtaccggacctaccagag | |
| | caaggatcagacgcttggggataaaaaggattttgatgctttcgtatcctatgcaaaatggagctcttttccaagtgaggc | |
| | cacttcatctctgagtgaagaacacttggccctgagcctatttcctgatgttttagaaaacaaatatggatatagcctgtgtt | |
| | tgcttgaaagagatgtggctccaggaggagtgtatgcagaagacattgtgagcattattaagagaagcagaagaggaa | |
| | tatttatcttgagccccaactatgtcaatggacccagtatctttgaactacaagcagcagtgaatcttgccttggatgatca | |
| | aacactgaaactcattttaattaagttctgttacttccaagagccagagtctctacctcatctcgtgaaaaaagctctcagg | |
| | gttttgcccacagttacttggagaggcttaaaatcagttcctcccaattctaggttctgggccaaaatgcgctaccacatg | |
| | cctgtgaaaaactctcagggattcacgtggaaccagctcagaattacctctaggatttttcagtggaaaggactcagtag | |
| | aacagaaaccactgggaggagctcccagcctaaggaatggtgataa | |
| CAR 20A10N | atggccttaccagtgaccgccttgctcctgccgctggccttgctgctccacgccgccaggccgcaggtgcagctggtt | 161 |
| CD28-CD28-CD28 | gaatctggcggcggacttgtgaagcctggcggatctctgagactgagctgtgccgccagcggcttcacctttagcacat | |
| CD3 | acgccatgagctggatcagacaggcccctggcaaaggcctggaatgggtggcgtctattggcagagccggcagcac | |
| 3XFOXP3NFAT | ctactacagcgactctgtgaagggcagattcaccatcagccgggacaacgccaagaacagcctgtacctgcagatga | |
| mCMV | actccctgagagccgaggacaccgccgtgtactattgtgccagaggacccatctacaacgactacgacgagttcgcct | |
| IL18Ralpha-T2A- | attggggccagggcacactggtcacagtttctagcggcggtggcggaagcggaggcggtggctccggtggcggag | |
| IL18Rb | gcagcgaaattgtgctgacacagagccccgccacactgtcactttctccaggcgaaagagccacactgagctgcaag | |
| | agcagccagagcgtgctgtactccagcaaccagaagaactacctggcctggtatcagcagaagcccggccaagctc | |
| | ctcggctgctgatctattgggccagcacaagagagagcggcatccctgccagattttctggcagcggctctggcaccg | |
| | atttcaccctgaccataagcagcctggaacctgaggacttcgccgtgtattactgccaccagtacctgagcagcctgac | |
| | ctttggcggaggcaccaaggtggaaatcaagcggaagcacctgtgtccttctccactgttccccggccctagcaagcc | |
| | tttctgggtcctggtggtggtgggcggagtgctggcctgctacagcctgctggtgaccgtggcctttatcatcttctgggt | |
| | gaggagtaagaggagcaggctcctgcacagtgactacatgaacatgactcctagaagacctgggcctaccagaaag | |
| | cattaccagccctatgccccaccacgcgacttcgcagcctatcgctccagagtgaagttcagcaggagcgcagacgc | |
| | ccccgcgtacaagcagggccagaaccagctctataacgagctcaatctaggacgaagagaggagtacgatgttttgg | |
| | acaagagacgtggccgggaccctgagatggggggaaagccgagaaggaagaaccctcaggaaggcctgtacaat | |
| | gaactgcagaaagataagatggcggaggcctacagtgagattgggatgaaaggcgagcgccggaggggcaaggg | |
| | gcacgatggcctttaccagggtctcagtacagccaccaaggacacctacgacgcccttcacatgcaggccctgcccc | |
| | ctcgctgataagtttaaactgccagaacatttctctggcctaactggccggtaccggcttcattttttccatttactgcagag | |
| | gcttcattttttccatttactgcagaggcttcattttttccatttactgcagaactagttaggcgtgtacggtgggaggcctat | |
| | ataagcagagctcgtttagtgaaccgtcagatcgcctggagacgccatccacgctgttttgacctccatagaagacacc | |
| | gggaccgatccagcctcgagagacccaatgctagccaccatgaattgtagagaattacccttgaccctttgggtgctta | |
| | tatctgtaagcactgcagaatcttgtacttcacgtccccacattactgtggttgaaggggaacctttctatctgaaacattgc | |
| | tcgtgttcacttgcacatgagattgaaacaaccaccaaaagctggtacaaaagcagtggatcacaggaacatgtggag | |
| | ctgaacccaaggagttccagcagaattgctttgcatgattgtgttttggagttttggccagttgagttgaatgacacaggat | |
| | cttactttttccaaatgaaaaattatactcagaaatggaaattaaatgtcatcagaagaaataaacacagctgtttcactgaa | |
| | agacaagtaactagtaaaattgtggaagttaaaaaattttttcagataacctgtgaaaacagttactatcaaacactggtca | |
| | acagcacatcattgtataagaactgtaaaaagctactactggagaacaataaaaacccaacgataaagaagaacgccg | |
| | agtttgaagatcaggggtattactcctgcgtgcatttccttcatcataatggaaaactatttaatatcaccaaaaccttcaata | |
| | taacaatagtggaagatcgcagtaatatagttccggttcttcttggaccaaagcttaaccatgttgcagtggaattaggaa | |
| | aaaacgtaaggctcaactgctctgctttgctgaatgaagaggatgtaatttattggatgttcggggaagaaaatggatcg | |
| | gatcctaatatacatgaagagaaagaaatgagaattatgactccagaaggcaaatggcatgcttcaaaagtattgagaat | |
| | tgaaaatattggtgaaagcaatctaaatgttttatataattgcactgtggccagcacgggaggcacagacaccaaaagct | |
| | tcatcttggtgagaaaagcagacatggctgatatcccaggccacgtcttcacaagaggaatgatcatagctgttttgatct | |
| | tggtggcagtagtgtgcctagtgactgtgtgtgtcatttatagagttgacttggttctattttatagacatttaacgagaaga | |
| | gatgaaacattaacagatggaaaaacatatgatgcttttgtgtcttacctaaaagaatgccgacctgaaaatggagagga | |
| | gcacacctttgctgtggagattttgcccagggtgttggagaaacattttgggtataagttatgcatatttgaaagggatgta | |
| | gtgcctggaggagctgttgttgatgaaatccactcactgatagagaaaagccgaagactaatcattgtcctaagtaaaag | |
| | ttatatgtctaatgaggtcaggtatgaacttgaaagtggactccatgaagcattggtggaaagaaaaattaaaataatctta | |
| | attgaatttacacctgttactgacttcacattcttgccccaatcactaaagcttttgaaatctcacagagttctgaagtggaa | |
| | ggccgataaatctctttcttataactcaaggttctggaagaaccttctttacttaatgcctgcaaaaacagtcaagccaggt | |
| | agagacgaaccggaagtcttgcctgttctttccgagtctggaagcggtgagggaagaggtagcctgctcacgtgcggt | |
| | gatgtggaggagaaccctgggcctatgctctgtttgggctggatatttctttggcttgttgcaggagagcgaattaaagg | |
| | atttaatatttcaggttgttccacaaaaaaactcctttggacatattctacaaggagtgaagaggaatttgtcttattttgtgat | |
| | ttaccagagccacagaaatcacatttctgccacagaaatcgactctcaccaaaacaagtccctgagcacctgcccttcat | |
| | gggtagtaacgacctatctgatgtccaatggtaccaacaaccttcgaatggagatccattagaggacattaggaaaagc | |
| | tatcctcacatcattcaggacaaatgtacccttcactttttgaccccaggggtgaataattctgggtcatatatttgtagacc | |
| | caagatgattaagagcccctatgatgtagcctgttgtgtcaagatgattttagaagttaagccccagacaaatgcatcctg | |
| | tgagtattccgcatcacataagcaagacctacttcttgggagcactggctctatttcttgccccagtctcagctgccaaagt | |
| | gatgcacaaagtccagcggtaacctggtacaagaatggaaaactcctctctgtggaaaggagcaaccgaatcgtagtg | |
| | gatgaagtttatgactatcaccagggcacatatgtatgtgattacactcagtcggatactgtgagttcgtggacagtcaga | |
| | gctgttgttcaagtgagaaccattgtgggagacactaaactcaaaccagatattctggatcctgtcgaggacacactgga | |
| | agtagaacttggaaagcctttaactattagctgcaaagcacgatttggctttgaaagggtctttaaccctgtcataaaatgg | |
| | tacatcaaagattctgacctagagtgggaagtctcagtacctgaggcgaaaagtattaaatccactttaaaggatgaaat | |
| | cattgagcgtaatatcatcttggaaaaagtcactcagcgtgatcttcgcaggaagtttgtttgctttgtccagaactccattg | |
| | gaaacacaacccagtccgtccaactgaaagaaaagagaggagtggtgctcctgtacatcctgcttggcaccatcggg | |
| | accctggtggccgtgctggcggcgagtgccctcctctacaggcactggattgaaatagtgctgctgtaccggacctac | |
| | cagagcaaggatcagacgcttggggataaaaaggattttgatgctttcgtatcctatgcaaaatggagctcttttccaagt | |
| | gaggccacttcatctctgagtgaagaacacttggccctgagcctatttcctgatgttttagaaaacaaatatggatatagc | |
| | ctgtgtttgcttgaaagagatgtggctccaggaggagtgtatgcagaagacattgtgagcattattaagagaagcagaa | |
| | gaggaatatttatcttgagccccaactatgtcaatggacccagtatctttgaactacaagcagcagtgaatcttgccttgg | |
| | atgatcaaacactgaaactcattttaattaagttctgttacttccaagagccagagtctctacctcatctcgtgaaaaaagct | |
| | ctcagggttttgcccacagttacttggagaggcttaaaatcagttcctcccaattctaggttctgggccaaaatgcgctac | |
| | cacatgcctgtgaaaaactctcagggattcacgtggaaccagctcagaattacctctaggatttttcagtggaaaggact | |
| | cagtagaacagaaaccactgggaggagctcccagcctaaggaatggtgataa | |
| CAR 20A10N | atggccttaccagtgaccgccttgctcctgccgctggccttgctgctccacgccgccaggccgcaggtgcagctggtt | 162 |
| CD28-CD28-CD28 | gaatctggcggcggacttgtgaagcctggcggatctctgagactgagctgtgccgccagcggcttcacctttagcacat | |
| CD3 6XIL2NFAT | acgccatgagctggatcagacaggcccctggcaaaggcctggaatgggtggcgtctattggcagagccggcagcac | |
| mCMV | ctactacagcgactctgtgaagggcagattcaccatcagccgggacaacgccaagaacagcctgtacctgcagatga | |
| IL18Ralpha-T2A- | actccctgagagccgaggacaccgccgtgtactattgtgccagaggacccatctacaacgactacgacgagttcgcct | |
| IL18Rb | attggggccagggcacactggtcacagtttctagcggcggtggcggaagcggaggcggtggctccggtggcggag | |
| | gcagcgaaattgtgctgacacagagccccgccacactgtcactttctccaggcgaaagagccacactgagctgcaag | |
| | agcagccagagcgtgctgtactccagcaaccagaagaactacctggcctggtatcagcagaagcccggccaagctc | |
| | ctcggctgctgatctattgggccagcacaagagagagcggcatccctgccagattttctggcagcggctctggcaccg | |
| | atttcaccctgaccataagcagcctggaacctgaggacttcgccgtgtattactgccaccagtacctgagcagcctgac | |
| | ctttggcggaggcaccaaggtggaaatcaagcggaagcacctgtgtccttctccactgttccccggccctagcaagcc | |
| | tttctgggtcctggtggtggtgggcggagtgctggcctgctacagcctgctggtgaccgtggcctttatcatcttctgggt | |
| | gaggagtaagaggagcaggctcctgcacagtgactacatgaacatgactcctagaagacctgggcctaccagaaag | |
| | cattaccagccctatgccccaccacgcgacttcgcagcctatcgctccagagtgaagttcagcaggagcgcagacgc | |
| | ccccgcgtacaagcagggccagaaccagctctataacgagctcaatctaggacgaagagaggagtacgatgttttgg | |
| | acaagagacgtggccgggaccctgagatggggggaaagccgagaaggaagaaccctcaggaaggcctgtacaat | |
| | gaactgcagaaagataagatggcggaggcctacagtgagattgggatgaaaggcgagcgccggaggggcaaggg | |
| | gcacgatggcctttaccagggtctcagtacagccaccaaggacacctacgacgcccttcacatgcaggccctgcccc | |
| | ctcgctgataagtttaaactgccagaacatttctctggcctaactggccggtaccggaggaaaaactgtttcatacagaa | |
| | ggcgtggaggaaaaactgtttcatacagaaggcgtggaggaaaaactgtttcatacagaaggcgtggaggaaaaact | |
| | gtttcatacagaaggcgtggaggaaaaactgtttcatacagaaggcgtggaggaaaaactgtttcatacagaaggcgt | |
| | actagttaggcgtgtacggtgggaggcctatataagcagagctcgtttagtgaaccgtcagatcgcctggagacgccat | |
| | ccacgctgttttgacctccatagaagacaccgggaccgatccagcctcgagagacccaatgctagccaccatgaatt | |
| | gtagagaattacccttgaccctttgggtgcttatatctgtaagcactgcagaatcttgtacttcacgtccccacattactgtg | |
| | gttgaaggggaacctttctatctgaaacattgctcgtgttcacttgcacatgagattgaaacaaccaccaaaagctggtac | |
| | aaaagcagtggatcacaggaacatgtggagctgaacccaaggagttccagcagaattgctttgcatgattgtgttttgga | |
| | gttttggccagttgagttgaatgacacaggatcttactttttccaaatgaaaaattatactcagaaatggaaattaaatgtcat | |
| | cagaagaaataaacacagctgtttcactgaaagacaagtaactagtaaaattgtggaagttaaaaaattttttcagataac | |
| | ctgtgaaaacagttactatcaaacactggtcaacagcacatcattgtataagaactgtaaaaagctactactggagaaca | |
| | ataaaaacccaacgataaagaagaacgccgagtttgaagatcaggggtattactcctgcgtgcatttccttcatcataat | |
| | ggaaaactatttaatatcaccaaaaccttcaatataacaatagtggaagatcgcagtaatatagttccggttcttcttggac | |
| | caaagcttaaccatgttgcagtggaattaggaaaaaacgtaaggctcaactgctctgctttgctgaatgaagaggatgta | |
| | atttattggatgttcggggaagaaaatggatcggatcctaatatacatgaagagaaagaaatgagaattatgactccaga | |
| | aggcaaatggcatgcttcaaaagtattgagaattgaaaatattggtgaaagcaatctaaatgttttatataattgcactgtg | |
| | gccagcacgggaggcacagacaccaaaagcttcatcttggtgagaaaagcagacatggctgatatcccaggccacg | |
| | tcttcacaagaggaatgatcatagctgttttgatcttggtggcagtagtgtgcctagtgactgtgtgtgtcatttatagagtt | |
| | gacttggttctattttatagacatttaacgagaagagatgaaacattaacagatggaaaaacatatgatgcttttgtgtctta | |
| | cctaaaagaatgccgacctgaaaatggagaggagcacacctttgctgtggagattttgcccagggtgttggagaaaca | |
| | ttttgggtataagttatgcatatttgaaagggatgtagtgcctggaggagctgttgttgatgaaatccactcactgatagag | |
| | aaaagccgaagactaatcattgtcctaagtaaaagttatatgtctaatgaggtcaggtatgaacttgaaagtggactccat | |
| | gaagcattggtggaaagaaaaattaaaataatcttaattgaatttacacctgttactgacttcacattcttgccccaatcact | |
| | aaagcttttgaaatctcacagagttctgaagtggaaggccgataaatctctttcttataactcaaggttctggaagaacctt | |
| | ctttacttaatgcctgcaaaaacagtcaagccaggtagagacgaaccggaagtcttgcctgttctttccgagtctggaag | |
| | cggtgagggaagaggtagcctgctcacgtgcggtgatgtggaggagaaccctgggcctatgctctgtttgggctggat | |
| | attttttggcttgttgcaggagagcgaattaaaggatttaatatttcaggttgttccacaaaaaaactcctttggacatattct | |
| | acaaggagtgaagaggaatttgtcttattttgtgatttaccagagccacagaaatcacatttctgccacagaaatcgactct | |
| | caccaaaacaagtccctgagcacctgcccttcatgggtagtaacgacctatctgatgtccaatggtaccaacaaccttcg | |
| | aatggagatccattagaggacattaggaaaagctatcctcacatcattcaggacaaatgtacccttcactttttgacccca | |
| | ggggtgaataattctgggtcatatatttgtagacccaagatgattaagagcccctatgatgtagcctgttgtgtcaagatg | |
| | attttagaagttaagccccagacaaatgcatcctgtgagtattccgcatcacataagcaagacctacttcttgggagcact | |
| | ggctctatttcttgccccagtctcagctgccaaagtgatgcacaaagtccagcggtaacctggtacaagaatggaaaac | |
| | tcctctctgtggaaaggagcaaccgaatcgtagtggatgaagtttatgactatcaccagggcacatatgtatgtgattaca | |
| | ctcagtcggatactgtgagttcgtggacagtcagagctgttgttcaagtgagaaccattgtgggagacactaaactcaaa | |
| | ccagatattctggatcctgtcgaggacacactggaagtagaacttggaaagcctttaactattagctgcaaagcacgattt | |
| | ggctttgaaagggtctttaaccctgtcataaaatggtacatcaaagattctgacctagagtgggaagtctcagtacctgag | |
| | gcgaaaagtattaaatccactttaaaggatgaaatcattgagcgtaatatcatcttggaaaaagtcactcagcgtgatcttc | |
| | gcaggaagtttgtttgctttgtccagaactccattggaaacacaacccagtccgtccaactgaaagaaaagagaggagt | |
| | ggtgctcctgtacatcctgcttggcaccatcgggaccctggtggccgtgctggcggcgagtgccctcctctacaggca | |
| | ctggattgaaatagtgctgctgtaccggacctaccagagcaaggatcagacgcttggggataaaaaggattttgatgctt | |
| | tcgtatcctatgcaaaatggagctcttttccaagtgaggccacttcatctctgagtgaagaacacttggccctgagcctatt | |
| | tcctgatgttttagaaaacaaatatggatatagcctgtgtttgcttgaaagagatgtggctccaggaggagtgtatgcaga | |
| | agacattgtgagcattattaagagaagcagaagaggaatatttatcttgagccccaactatgtcaatggacccagtatctt | |
| | tgaactacaagcagcagtgaatcttgccttggatgatcaaacactgaaactcattttaattaagttctgttacttccaagagc | |
| | cagagtctctacctcatctcgtgaaaaaagctctcagggttttgcccacagttacttggagaggcttaaaatcagttcctcc | |
| | caattctaggttctgggccaaaatgcgctaccacatgcctgtgaaaaactctcagggattcacgtggaaccagctcaga | |
| | attacctctaggatttttcagtggaaaggactcagtagaacagaaaccactgggaggagctcccagcctaaggaatggt | |
| | gataa | |
| CAR 20A10N | atggccttaccagtgaccgccttgctcctgccgctggccttgctgctccacgccgccaggccgcaggtgcagctggtt | 163 |
| CD28-CD28-CD28 | gaatctggcggcggacttgtgaagcctggcggatctctgagactgagctgtgccgccagcggcttcacctttagcacat | |
| CD3 3XIL2NFAT | acgccatgagctggatcagacaggcccctggcaaaggcctggaatgggtggcgtctattggcagagccggcagcac | |
| mCMV | ctactacagcgactctgtgaagggcagattcaccatcagccgggacaacgccaagaacagcctgtacctgcagatga | |
| IL18Ralpha-T2A- | actccctgagagccgaggacaccgccgtgtactattgtgccagaggacccatctacaacgactacgacgagttcgcct | |
| IL18Rb | attggggccagggcacactggtcacagtttctagcggcggtggcggaagcggaggcggtggctccggtggcggag | |
| | gcagcgaaattgtgctgacacagagccccgccacactgtcactttctccaggcgaaagagccacactgagctgcaag | |
| | agcagccagagcgtgctgtactccagcaaccagaagaactacctggcctggtatcagcagaagcccggccaagctc | |
| | ctcggctgctgatctattgggccagcacaagagagagcggcatccctgccagattttctggcagcggctctggcaccg | |
| | atttcaccctgaccataagcagcctggaacctgaggacttcgccgtgtattactgccaccagtacctgagcagcctgac | |
| | ctttggcggaggcaccaaggtggaaatcaagcggaagcacctgtgtccttctccactgttccccggccctagcaagcc | |
| | tttctgggtcctggtggtggtgggcggagtgctggcctgctacagcctgctggtgaccgtggcctttatcatcttctgggt | |
| | gaggagtaagaggagcaggctcctgcacagtgactacatgaacatgactcctagaagacctgggcctaccagaaag | |
| | cattaccagccctatgccccaccacgcgacttcgcagcctatcgctccagagtgaagttcagcaggagcgcagacgc | |
| | ccccgcgtacaagcagggccagaaccagctctataacgagctcaatctaggacgaagagaggagtacgatgttttgg | |
| | acaagagacgtggccgggaccctgagatggggggaaagccgagaaggaagaaccctcaggaaggcctgtacaat | |
| | gaactgcagaaagataagatggcggaggcctacagtgagattgggatgaaaggcgagcgccggaggggcaaggg | |
| | gcacgatggcctttaccagggtctcagtacagccaccaaggacacctacgacgcccttcacatgcaggccctgcccc | |
| | ctcgctgataagtttaaactgccagaacatttctctggcctaactggccggtaccggaggaaaaactgtttcatacagaa | |
| | ggcgtggaggaaaaactgtttcatacagaaggcgtggaggaaaaactgtttcatacagaaggcgtactagttaggcgt | |
| | gtacggtgggaggcctatataagcagagctcgtttagtgaaccgtcagatcgcctggagacgccatccacgctgttttg | |
| | acctccatagaagacaccgggaccgatccagcctcgagagacccaatgctagccaccatgaattgtagagaattacc | |
| | cttgaccctttgggtgcttatatctgtaagcactgcagaatcttgtacttcacgtccccacattactgtggttgaaggggaa | |
| | cctttctatctgaaacattgctcgtgttcacttgcacatgagattgaaacaaccaccaaaagctggtacaaaagcagtgga | |
| | tcacaggaacatgtggagctgaacccaaggagttccagcagaattgctttgcatgattgtgttttggagttttggccagtt | |
| | gagttgaatgacacaggatcttactttttccaaatgaaaaattatactcagaaatggaaattaaatgtcatcagaagaaata | |
| | aacacagctgtttcactgaaagacaagtaactagtaaaattgtggaagttaaaaaattttttcagataacctgtgaaaaca | |
| | gttactatcaaacactggtcaacagcacatcattgtataagaactgtaaaaagctactactggagaacaataaaaaccca | |
| | acgataaagaagaacgccgagtttgaagatcaggggtattactcctgcgtgcatttccttcatcataatggaaaactattt | |
| | aatatcaccaaaaccttcaatataacaatagtggaagatcgcagtaatatagttccggttcttcttggaccaaagcttaacc | |
| | atgttgcagtggaattaggaaaaaacgtaaggctcaactgctctgctttgctgaatgaagaggatgtaatttattggatgtt | |
| | cggggaagaaaatggatcggatcctaatatacatgaagagaaagaaatgagaattatgactccagaaggcaaatggc | |
| | atgcttcaaaagtattgagaattgaaaatattggtgaaagcaatctaaatgttttatataattgcactgtggccagcacggg | |
| | aggcacagacaccaaaagcttcatcttggtgagaaaagcagacatggctgatatcccaggccacgtcttcacaagag | |
| | gaatgatcatagctgttttgatcttggtggcagtagtgtgcctagtgactgtgtgtgtcatttatagagttgacttggttctatt | |
| | ttatagacatttaacgagaagagatgaaacattaacagatggaaaaacatatgatgcttttgtgtcttacctaaaagaatgc | |
| | cgacctgaaaatggagaggagcacacctttgctgtggagattttgcccagggtgttggagaaacattttgggtataagtt | |
| | atgcatatttgaaagggatgtagtgcctggaggagctgttgttgatgaaatccactcactgatagagaaaagccgaaga | |
| | ctaatcattgtcctaagtaaaagttatatgtctaatgaggtcaggtatgaacttgaaagtggactccatgaagcattggtgg | |
| | aaagaaaaattaaaataatcttaattgaatttacacctgttactgacttcacattcttgccccaatcactaaagcttttgaaatc | |
| | tcacagagttctgaagtggaaggccgataaatctctttcttataactcaaggttctggaagaaccttctttacttaatgcctg | |
| | caaaaacagtcaagccaggtagagacgaaccggaagtcttgcctgttctttccgagtctggaagcggtgagggaaga | |
| | ggtagcctgctcacgtgcggtgatgtggaggagaaccctgggcctatgctctgtttgggctggatatttctttggcttgtt | |
| | gcaggagagcgaattaaaggatttaatatttcaggttgttccacaaaaaaactcctttggacatattctacaaggagtgaa | |
| | gaggaatttgtcttattttgtgatttaccagagccacagaaatcacatttctgccacagaaatcgactctcaccaaaacaag | |
| | tccctgagcacctgcccttcatgggtagtaacgacctatctgatgtccaatggtaccaacaaccttcgaatggagatcca | |
| | ttagaggacattaggaaaagctatcctcacatcattcaggacaaatgtacccttcactttttgaccccaggggtgaataatt | |
| | ctgggtcatatatttgtagacccaagatgattaagagcccctatgatgtagcctgttgtgtcaagatgattttagaagttaag | |
| | ccccagacaaatgcatcctgtgagtattccgcatcacataagcaagacctacttcttgggagcactggctctatttcttgc | |
| | cccagtctcagctgccaaagtgatgcacaaagtccagcggtaacctggtacaagaatggaaaactcctctctgtggaa | |
| | aggagcaaccgaatcgtagtggatgaagtttatgactatcaccagggcacatatgtatgtgattacactcagtcggatac | |
| | tgtgagttcgtggacagtcagagctgttgttcaagtgagaaccattgtgggagacactaaactcaaaccagatattctgg | |
| | atcctgtcgaggacacactggaagtagaacttggaaagcctttaactattagctgcaaagcacgatttggctttgaaagg | |
| | gtctttaaccctgtcataaaatggtacatcaaagattctgacctagagtgggaagtctcagtacctgaggcgaaaagtatt | |
| | aaatccactttaaaggatgaaatcattgagcgtaatatcatcttggaaaaagtcactcagcgtgatcttcgcaggaagttt | |
| | gtttgctttgtccagaactccattggaaacacaacccagtccgtccaactgaaagaaaagagaggagtggtgctcctgt | |
| | acatcctgcttggcaccatcgggaccctggtggccgtgctggcggcgagtgccctcctctacaggcactggattgaaa | |
| | tagtgctgctgtaccggacctaccagagcaaggatcagacgcttggggataaaaaggattttgatgctttcgtatcctat | |
| | gcaaaatggagctcttttccaagtgaggccacttcatctctgagtgaagaacacttggccctgagcctatttcctgatgttt | |
| | tagaaaacaaatatggatatagcctgtgtttgcttgaaagagatgtggctccaggaggagtgtatgcagaagacattgtg | |
| | agcattattaagagaagcagaagaggaatatttatcttgagccccaactatgtcaatggacccagtatctttgaactacaa | |
| | gcagcagtgaatcttgccttggatgatcaaacactgaaactcattttaattaagttctgttacttccaagagccagagtctct | |
| | acctcatctcgtgaaaaaagctctcagggttttgcccacagttacttggagaggcttaaaatcagttcctcccaattctagg | |
| | ttctgggccaaaatgcgctaccacatgcctgtgaaaaactctcagggattcacgtggaaccagctcagaattacctctag | |
| | gatttttcagtggaaaggactcagtagaacagaaaccactgggaggagctcccagcctaaggaatggtgataa | |
| CAR 20A10N | atggccttaccagtgaccgccttgctcctgccgctggccttgctgctccacgccgccaggccgcaggtgcagctggtt | 164 |
| CD28-CD28-CD28 | gaatctggcggcggacttgtgaagcctggcggatctctgagactgagctgtgccgccagcggcttcacctttagcacat | |
| 1XX | acgccatgagctggatcagacaggcccctggcaaaggcctggaatgggtggcgtctattggcagagccggcagcac | |
| 6XFOXP3NFAT | ctactacagcgactctgtgaagggcagattcaccatcagccgggacaacgccaagaacagcctgtacctgcagatga | |
| mCMV | actccctgagagccgaggacaccgccgtgtactattgtgccagaggacccatctacaacgactacgacgagttcgcct | |
| IL18Ralpha-T2A- | attggggccagggcacactggtcacagtttctagcggcggtggcggaagcggaggcggtggctccggtggcggag | |
| IL18Rb | gcagcgaaattgtgctgacacagagccccgccacactgtcactttctccaggcgaaagagccacactgagctgcaag | |
| | agcagccagagcgtgctgtactccagcaaccagaagaactacctggcctggtatcagcagaagcccggccaagctc | |
| | ctcggctgctgatctattgggccagcacaagagagagcggcatccctgccagattttctggcagcggctctggcaccg | |
| | atttcaccctgaccataagcagcctggaacctgaggacttcgccgtgtattactgccaccagtacctgagcagcctgac | |
| | ctttggcggaggcaccaaggtggaaatcaagcggaagcacctgtgtccttctccactgttccccggccctagcaagcc | |
| | tttctgggtcctggtggtggtgggcggagtgctggcctgctacagcctgctggtgaccgtggcctttatcatcttctgggt | |
| | gaggagtaagaggagcaggctcctgcacagtgactacatgaacatgactcctagaagacctgggcctaccagaaag | |
| | cattaccagccctatgccccaccacgcgacttcgcagcctatcgctccagagtgaagttcagcaggagcgcagacgc | |
| | ccccgcgtacaagcagggccagaaccagctctataacgagctcaatctaggacgaagagaggagtacgatgttttgg | |
| | acaagagacgtggccgggaccctgagatggggggaaagccgagaaggaagaaccctcaggaaggcctgttcaat | |
| | gaactgcagaaagataagatggcggaggccttcagtgagattgggatgaaaggcgagcgccggaggggcaaggg | |
| | gcacgatggccttttccagggtctcagtacagccaccaaggacaccttcgacgcccttcacatgcaggccctgccccc | |
| | tcgctgataagtttaaactgccagaacattactctggcctaactggccggtaccggcttcattttttccatttactgcagagg | |
| | cttcattttttccatttactgcagaggcttcattttttccatttactgcagaggcttcattttttccatttactgcagaggcttcattt | |
| | tttccatttactgcagaggcttcattttttccatttactgcagaactagttaggcgtgtacggtgggaggcctatataagcag | |
| | agctcgtttagtgaaccgtcagatcgcctggagacgccatccacgctgttttgacctccatagaagacaccgggaccg | |
| | atccagcctcgagagacccaatgctagccaccatgaattgtagagaattacccttgaccctttgggtgcttatatctgtaa | |
| | gcactgcagaatcttgtacttcacgtccccacattactgtggttgaaggggaacctttctatctgaaacattgctcgtgttca | |
| | cttgcacatgagattgaaacaaccaccaaaagctggtacaaaagcagtggatcacaggaacatgtggagctgaaccc | |
| | aaggagttccagcagaattgctttgcatgattgtgttttggagttttggccagttgagttgaatgacacaggatcttacttttt | |
| | ccaaatgaaaaattatactcagaaatggaaattaaatgtcatcagaagaaataaacacagctgtttcactgaaagacaag | |
| | taactagtaaaattgtggaagttaaaaaattttttcagataacctgtgaaaacagttactatcaaacactggtcaacagcac | |
| | atcattgtataagaactgtaaaaagctactactggagaacaataaaaacccaacgataaagaagaacgccgagtttgaa | |
| | gatcaggggtattactcctgcgtgcatttccttcatcataatggaaaactatttaatatcaccaaaaccttcaatataacaat | |
| | agtggaagatcgcagtaatatagttccggttcttcttggaccaaagcttaaccatgttgcagtggaattaggaaaaaacgt | |
| | aaggctcaactgctctgctttgctgaatgaagaggatgtaatttattggatgttcggggaagaaaatggatcggatcctaa | |
| | tatacatgaagagaaagaaatgagaattatgactccagaaggcaaatggcatgcttcaaaagtattgagaattgaaaata | |
| | ttggtgaaagcaatctaaatgttttatataattgcactgtggccagcacgggaggcacagacaccaaaagcttcatcttg | |
| | gtgagaaaagcagacatggctgatatcccaggccacgtcttcacaagaggaatgatcatagctgttttgatcttggtggc | |
| | agtagtgtgcctagtgactgtgtgtgtcatttatagagttgacttggttctattttatagacatttaacgagaagagatgaaa | |
| | cattaacagatggaaaaacatatgatgcttttgtgtcttacctaaaagaatgccgacctgaaaatggagaggagcacacc | |
| | tttgctgtggagattttgcccagggtgttggagaaacattttgggtataagttatgcatatttgaaagggatgtagtgcctg | |
| | gaggagctgttgttgatgaaatccactcactgatagagaaaagccgaagactaatcattgtcctaagtaaaagttatatgt | |
| | ctaatgaggtcaggtatgaacttgaaagtggactccatgaagcattggtggaaagaaaaattaaaataatcttaattgaat | |
| | ttacacctgttactgacttcacattcttgccccaatcactaaagcttttgaaatctcacagagttctgaagtggaaggccgat | |
| | aaatctctttcttataactcaaggttctggaagaaccttctttacttaatgcctgcaaaaacagtcaagccaggtagagacg | |
| | aaccggaagtcttgcctgttctttccgagtctggaagcggtgagggaagaggtagcctgctcacgtgcggtgatgtgg | |
| | aggagaaccctgggcctatgctctgtttgggctggatatttctttggcttgttgcaggagagcgaattaaaggatttaatat | |
| | ttcaggttgttccacaaaaaaactcctttggacatattctacaaggagtgaagaggaatttgtcttattttgtgatttaccaga | |
| | gccacagaaatcacatttctgccacagaaatcgactctcaccaaaacaagtccctgagcacctgcccttcatgggtagt | |
| | aacgacctatctgatgtccaatggtaccaacaaccttcgaatggagatccattagaggacattaggaaaagctatcctca | |
| | catcattcaggacaaatgtacccttcactttttgaccccaggggtgaataattctgggtcatatatttgtagacccaagatg | |
| | attaagagcccctatgatgtagcctgttgtgtcaagatgattttagaagttaagccccagacaaatgcatcctgtgagtatt | |
| | ccgcatcacataagcaagacctacttcttgggagcactggctctatttcttgccccagtctcagctgccaaagtgatgca | |
| | caaagtccagcggtaacctggtacaagaatggaaaactcctctctgtggaaaggagcaaccgaatcgtagtggatgaa | |
| | gtttatgactatcaccagggcacatatgtatgtgattacactcagtcggatactgtgagttcgtggacagtcagagctgtt | |
| | gttcaagtgagaaccattgtgggagacactaaactcaaaccagatattctggatcctgtcgaggacacactggaagtag | |
| | aacttggaaagcctttaactattagctgcaaagcacgatttggctttgaaagggtctttaaccctgtcataaaatggtacat | |
| | caaagattctgacctagagtgggaagtctcagtacctgaggcgaaaagtattaaatccactttaaaggatgaaatcattg | |
| | agcgtaatatcatcttggaaaaagtcactcagcgtgatcttcgcaggaagtttgtttgctttgtccagaactccattggaaa | |
| | cacaacccagtccgtccaactgaaagaaaagagaggagtggtgctcctgtacatcctgcttggcaccatcgggaccct | |
| | ggtggccgtgctggcggcgagtgccctcctctacaggcactggattgaaatagtgctgctgtaccggacctaccagag | |
| | caaggatcagacgcttggggataaaaaggattttgatgctttcgtatcctatgcaaaatggagctcttttccaagtgaggc | |
| | cacttcatctctgagtgaagaacacttggccctgagcctatttcctgatgttttagaaaacaaatatggatatagcctgtgtt | |
| | tgcttgaaagagatgtggctccaggaggagtgtatgcagaagacattgtgagcattattaagagaagcagaagaggaa | |
| | tatttatcttgagccccaactatgtcaatggacccagtatctttgaactacaagcagcagtgaatcttgccttggatgatca | |
| | aacactgaaactcattttaattaagttctgttacttccaagagccagagtctctacctcatctcgtgaaaaaagctctcagg | |
| | gttttgcccacagttacttggagaggcttaaaatcagttcctcccaattctaggttctgggccaaaatgcgctaccacatg | |
| | cctgtgaaaaactctcagggattcacgtggaaccagctcagaattacctctaggatttttcagtggaaaggactcagtag | |
| | aacagaaaccactgggaggagctcccagcctaaggaatggtgataa | |
| CAR 20A10N | atggccttaccagtgaccgccttgctcctgccgctggccttgctgctccacgccgccaggccgcaggtgcagctggtt | 165 |
| CD28-CD28-CD28 | gaatctggcggcggacttgtgaagcctggcggatctctgagactgagctgtgccgccagcggcttcacctttagcacat | |
| 1XX | acgccatgagctggatcagacaggcccctggcaaaggcctggaatgggtggcgtctattggcagagccggcagcac | |
| 3XFOXP3NFAT | ctactacagcgactctgtgaagggcagattcaccatcagccgggacaacgccaagaacagcctgtacctgcagatga | |
| mCMV | actccctgagagccgaggacaccgccgtgtactattgtgccagaggacccatctacaacgactacgacgagttcgcct | |
| IL18Ralpha-T2A- | attggggccagggcacactggtcacagtttctagcggcggtggcggaagcggaggcggtggctccggtggcggag | |
| IL18Rb | gcagcgaaattgtgctgacacagagccccgccacactgtcactttctccaggcgaaagagccacactgagctgcaag | |
| | agcagccagagcgtgctgtactccagcaaccagaagaactacctggcctggtatcagcagaagcccggccaagctc | |
| | ctcggctgctgatctattgggccagcacaagagagagcggcatccctgccagattttctggcagcggctctggcaccg | |
| | atttcaccctgaccataagcagcctggaacctgaggacttcgccgtgtattactgccaccagtacctgagcagcctgac | |
| | ctttggcggaggcaccaaggtggaaatcaagcggaagcacctgtgtccttctccactgttccccggccctagcaagcc | |
| | tttctgggtcctggtggtggtgggcggagtgctggcctgctacagcctgctggtgaccgtggcctttatcatcttctgggt | |
| | gaggagtaagaggagcaggctcctgcacagtgactacatgaacatgactcctagaagacctgggcctaccagaaag | |
| | cattaccagccctatgccccaccacgcgacttcgcagcctatcgctccagagtgaagttcagcaggagcgcagacgc | |
| | ccccgcgtacaagcagggccagaaccagctctataacgagctcaatctaggacgaagagaggagtacgatgttttgg | |
| | acaagagacgtggccgggaccctgagatggggggaaagccgagaaggaagaaccctcaggaaggcctgttcaat | |
| | gaactgcagaaagataagatggcggaggccttcagtgagattgggatgaaaggcgagcgccggaggggcaaggg | |
| | gcacgatggccttttccagggtctcagtacagccaccaaggacaccttcgacgcccttcacatgcaggccctgccccc | |
| | tcgctgataagtttaaactgccagaacattactctggcctaactggccggtaccggcttcattttttccatttactgcagagg | |
| | cttcattttttccatttactgcagaggcttcattttttccatttactgcagaactagttaggcgtgtacggtgggaggcctatat | |
| | aagcagagctcgtttagtgaaccgtcagatcgcctggagacgccatccacgctgttttgacctccatagaagacaccg | |
| | ggaccgatccagcctcgagagacccaatgctagccaccatgaattgtagagaattacccttgaccctttgggtgcttat | |
| | atctgtaagcactgcagaatcttgtacttcacgtccccacattactgtggttgaaggggaacctttctatctgaaacattgct | |
| | cgtgttcacttgcacatgagattgaaacaaccaccaaaagctggtacaaaagcagtggatcacaggaacatgtggagc | |
| | tgaacccaaggagttccagcagaattgctttgcatgattgtgttttggagttttggccagttgagttgaatgacacaggatc | |
| | ttactttttccaaatgaaaaattatactcagaaatggaaattaaatgtcatcagaagaaataaacacagctgtttcactgaaa | |
| | gacaagtaactagtaaaattgtggaagttaaaaaattttttcagataacctgtgaaaacagttactatcaaacactggtcaa | |
| | cagcacatcattgtataagaactgtaaaaagctactactggagaacaataaaaacccaacgataaagaagaacgccga | |
| | gtttgaagatcaggggtattactcctgcgtgcatttccttcatcataatggaaaactatttaatatcaccaaaaccttcaatat | |
| | aacaatagtggaagatcgcagtaatatagttccggttcttcttggaccaaagcttaaccatgttgcagtggaattaggaaa | |
| | aaacgtaaggctcaactgctctgctttgctgaatgaagaggatgtaatttattggatgttcggggaagaaaatggatc gg | |
| | atcctaatatacatgaagagaaagaaatgagaattatgactccagaaggcaaatggcatgcttcaaaagtattgagaatt | |
| | gaaaatattggtgaaagcaatctaaatgttttatataattgcactgtggccagcacgggaggcacagacaccaaaagctt | |
| | catcttggtgagaaaagcagacatggctgatatcccaggccacgtcttcacaagaggaatgatcatagctgttttgatctt | |
| | ggtggcagtagtgtgcctagtgactgtgtgtgtcatttatagagttgacttggttctattttatagacatttaacgagaagag | |
| | atgaaacattaacagatggaaaaacatatgatgcttttgtgtcttacctaaaagaatgccgacctgaaaatggagaggag | |
| | cacacctttgctgtggagattttgcccagggtgttggagaaacattttgggtataagttatgcatatttgaaagggatgtag | |
| | tgcctggaggagctgttgttgatgaaatccactcactgatagagaaaagccgaagactaatcattgtcctaagtaaaagt | |
| | tatatgtctaatgaggtcaggtatgaacttgaaagtggactccatgaagcattggtggaaagaaaaattaaaataatctta | |
| | attgaatttacacctgttactgacttcacattcttgccccaatcactaaagcttttgaaatctcacagagttctgaagtggaa | |
| | ggccgataaatctctttcttataactcaaggttctggaagaaccttctttacttaatgcctgcaaaaacagtcaagccaggt | |
| | agagacgaaccggaagtcttgcctgttctttccgagtctggaagcggtgagggaagaggtagcctgctcacgtgcggt | |
| | gatgtggaggagaaccctgggcctatgctctgtttgggctggatatttctttggcttgttgcaggagagcgaattaaagg | |
| | atttaatatttcaggttgttccacaaaaaaactcctttggacatattctacaaggagtgaagaggaatttgtcttattttgtgat | |
| | ttaccagagccacagaaatcacatttctgccacagaaatcgactctcaccaaaacaagtccctgagcacctgcccttcat | |
| | gggtagtaacgacctatctgatgtccaatggtaccaacaaccttcgaatggagatccattagaggacattaggaaaagc | |
| | tatcctcacatcattcaggacaaatgtacccttcactttttgaccccaggggtgaataattctgggtcatatatttgtagacc | |
| | caagatgattaagagcccctatgatgtagcctgttgtgtcaagatgattttagaagttaagccccagacaaatgcatcctg | |
| | tgagtattccgcatcacataagcaagacctacttcttgggagcactggctctatttcttgccccagtctcagctgccaaagt | |
| | gatgcacaaagtccagcggtaacctggtacaagaatggaaaactcctctctgtggaaaggagcaaccgaatcgtagtg | |
| | gatgaagtttatgactatcaccagggcacatatgtatgtgattacactcagtcggatactgtgagttcgtggacagtcaga | |
| | gctgttgttcaagtgagaaccattgtgggagacactaaactcaaaccagatattctggatcctgtcgaggacacactgga | |
| | agtagaacttggaaagcctttaactattagctgcaaagcacgatttggctttgaaagggtctttaaccctgtcataaaatgg | |
| | tacatcaaagattctgacctagagtgggaagtctcagtacctgaggcgaaaagtattaaatccactttaaaggatgaaat | |
| | cattgagcgtaatatcatcttggaaaaagtcactcagcgtgatcttcgcaggaagtttgtttgctttgtccagaactccattg | |
| | gaaacacaacccagtccgtccaactgaaagaaaagagaggagtggtgctcctgtacatcctgcttggcaccatcggg | |
| | accctggtggccgtgctggcggcgagtgccctcctctacaggcactggattgaaatagtgctgctgtaccggacctac | |
| | cagagcaaggatcagacgcttggggataaaaaggattttgatgctttcgtatcctatgcaaaatggagctcttttccaagt | |
| | gaggccacttcatctctgagtgaagaacacttggccctgagcctatttcctgatgttttagaaaacaaatatggatatagc | |
| | ctgtgtttgcttgaaagagatgtggctccaggaggagtgtatgcagaagacattgtgagcattattaagagaagcagaa | |
| | gaggaatatttatcttgagccccaactatgtcaatggacccagtatctttgaactacaagcagcagtgaatcttgccttgg | |
| | atgatcaaacactgaaactcattttaattaagttctgttacttccaagagccagagtctctacctcatctc gtgaaaaaagct | |
| | ctcagggttttgcccacagttacttggagaggcttaaaatcagttcctcccaattctaggttctgggccaaaatgcgctac | |
| | cacatgcctgtgaaaaactctcagggattcacgtggaaccagctcagaattacctctaggatttttcagtggaaaggact | |
| | cagtagaacagaaaccactgggaggagctcccagcctaaggaatggtgataa | |
| CAR 20A10N | atggccttaccagtgaccgccttgctcctgccgctggccttgctgctccacgccgccaggccgcaggtgcagctggtt | 166 |
| CD28-CD28-CD28 | gaatctggcggcggacttgtgaagcctggcggatctctgagactgagctgtgccgccagcggcttcacctttagcacat | |
| 1XX 6XIL2NFAT | acgccatgagctggatcagacaggcccctggcaaaggcctggaatgggggcgtctattggcagagccggcagcac | |
| mCMV | ctactacagcgactctgtgaagggcagattcaccatcagccgggacaacgccaagaacagcctgtacctgcagatga | |
| IL18Ralpha-T2A- | actccctgagagccgaggacaccgccgtgtactattgtgccagaggacccatctacaacgactacgacgagttcgcct | |
| IL18Rb | attggggccagggcacactggtcacagtttctagcggcggtggcggaagcggaggcggtggctccggtggcggag | |
| | gcagcgaaattgtgctgacacagagccccgccacactgtcactttctccaggcgaaagagccacactgagctgcaag | |
| | agcagccagagcgtgctgtactccagcaaccagaagaactacctggcctggtatcagcagaagcccggccaagctc | |
| | ctcggctgctgatctattgggccagcacaagagagagcggcatccctgccagattttctggcagcggctctggcaccg | |
| | atttcaccctgaccataagcagcctggaacctgaggacttcgccgtgtattactgccaccagtacctgagcagcctgac | |
| | ctttggcggaggcaccaaggtggaaatcaagcggaagcacctgtgtccttctccactgttccccggccctagcaagcc | |
| | tttctgggtcctggtggtggtgggcggagtgctggcctgctacagcctgctggtgaccgtggcctttatcatcttctgggt | |
| | gaggagtaagaggagcaggctcctgcacagtgactacatgaacatgactcctagaagacctgggcctaccagaaag | |
| | cattaccagccctatgccccaccacgcgacttcgcagcctatcgctccagagtgaagttcagcaggagcgcagacgc | |
| | ccccgcgtacaagcagggccagaaccagctctataacgagctcaatctaggacgaagagaggagtacgatgttttgg | |
| | acaagagacgtggccgggaccctgagatggggggaaagccgagaaggaagaaccctcaggaaggcctgttcaat | |
| | gaactgcagaaagataagatggcggaggccttcagtgagattgggatgaaaggcgagcgccggaggggcaaggg | |
| | gcacgatggccttttccagggtctcagtacagccaccaaggacaccttcgacgcccttcacatgcaggccctgccccc | |
| | tcgctgataagtttaaactgccagaacattactctggcctaactggccggtaccggaggaaaaactgtttcatacagaag | |
| | gcgtggaggaaaaactgtttcatacagaaggcgtggaggaaaaactgtttcatacagaaggcgtggaggaaaaactg | |
| | tttcatacagaaggcgtggaggaaaaactgtttcatacagaaggcgtggaggaaaaactgtttcatacagaaggcgtac | |
| | tagttaggcgtgtacggtgggaggcctatataagcagagctcgtttagtgaaccgtcagatcgcctggagacgccatcc | |
| | acgctgttttgacctccatagaagacaccgggaccgatccagcctcgagagacccaatgctagccaccatgaattgta | |
| | gagaattacccttgaccctttgggtgcttatatctgtaagcactgcagaatcttgtacttcacgtccccacattactgtggtt | |
| | gaaggggaacctttctatctgaaacattgctcgtgttcacttgcacatgagattgaaacaaccaccaaaagctggtacaa | |
| | aagcagtggatcacaggaacatgtggagctgaacccaaggagttccagcagaattgctttgcatgattgtgttttggagt | |
| | tttggccagttgagttgaatgacacaggatcttactttttccaaatgaaaaattatactcagaaatggaaattaaatgtcatc | |
| | agaagaaataaacacagctgtttcactgaaagacaagtaactagtaaaattgtggaagttaaaaaattttttcagataacct | |
| | gtgaaaacagttactatcaaacactggtcaacagcacatcattgtataagaactgtaaaaagctactactggagaacaat | |
| | aaaaacccaacgataaagaagaacgccgagtttgaagatcaggggtattactcctgcgtgcatttccttcatcataatgg | |
| | aaaactatttaatatcaccaaaaccttcaatataacaatagtggaagatcgcagtaatatagttccggttcttcttggaccaa | |
| | agcttaaccatgttgcagtggaattaggaaaaaacgtaaggctcaactgctctgctttgctgaatgaagaggatgtaattt | |
| | attggatgttcggggaagaaaatggatcggatcctaatatacatgaagagaaagaaatgagaattatgactccagaagg | |
| | caaatggcatgcttcaaaagtattgagaattgaaaatattggtgaaagcaatctaaatgttttatataattgcactgtggcca | |
| | gcacgggaggcacagacaccaaaagcttcatcttggtgagaaaagcagacatggctgatatcccaggccacgtcttc | |
| | acaagaggaatgatcatagctgttttgatcttggtggcagtagtgtgcctagtgactgtgtgtgtcatttatagagttgactt | |
| | ggttctattttatagacatttaacgagaagagatgaaacattaacagatggaaaaacatatgatgcttttgtgtcttacctaa | |
| | aagaatgccgacctgaaaatggagaggagcacacctttgctgtggagattttgcccagggtgttggagaaacattttgg | |
| | gtataagttatgcatatttgaaagggatgtagtgcctggaggagctgttgttgatgaaatccactcactgatagagaaaag | |
| | ccgaagactaatcattgtcctaagtaaaagttatatgtctaatgaggtcaggtatgaacttgaaagtggactccatgaagc | |
| | attggtggaaagaaaaattaaaataatcttaattgaatttacacctgttactgacttcacattcttgccccaatcactaaagct | |
| | tttgaaatctcacagagttctgaagtggaaggccgataaatctctttcttataactcaaggttctggaagaaccttctttactt | |
| | aatgcctgcaaaaacagtcaagccaggtagagacgaaccggaagtcttgcctgttctttccgagtctggaagcggtga | |
| | gggaagaggtagcctgctcacgtgcggtgatgtggaggagaaccctgggcctatgctctgtttgggctggatatttcttt | |
| | ggcttgttgcaggagagcgaattaaaggatttaatatttcaggttgttccacaaaaaaactcctttggacatattctacaag | |
| | gagtgaagaggaatttgtcttattttgtgatttaccagagccacagaaatcacatttctgccacagaaatcgactctcacca | |
| | aaacaagtccctgagcacctgcccttcatgggtagtaacgacctatctgatgtccaatggtaccaacaaccttcgaatgg | |
| | agatccattagaggacattaggaaaagctatcctcacatcattcaggacaaatgtacccttcactttttgaccccaggggt | |
| | gaataattctgggtcatatatttgtagacccaagatgattaagagcccctatgatgtagcctgttgtgtcaagatgattttag | |
| | aagttaagccccagacaaatgcatcctgtgagtattccgcatcacataagcaagacctacttcttgggagcactggctct | |
| | atttcttgccccagtctcagctgccaaagtgatgcacaaagtccagcggtaacctggtacaagaatggaaaactcctctc | |
| | tgtggaaaggagcaaccgaatcgtagtggatgaagtttatgactatcaccagggcacatatgtatgtgattacactcagt | |
| | cggatactgtgagttcgtggacagtcagagctgttgttcaagtgagaaccattgtgggagacactaaactcaaaccaga | |
| | tattctggatcctgtcgaggacacactggaagtagaacttggaaagcctttaactattagctgcaaagcacgatttggcttt | |
| | gaaagggtctttaaccctgtcataaaatggtacatcaaagattctgacctagagtgggaagtctcagtacctgaggcgaa | |
| | aagtattaaatccactttaaaggatgaaatcattgagcgtaatatcatcttggaaaaagtcactcagcgtgatcttcgcagg | |
| | aagtttgtttgctttgtccagaactccattggaaacacaacccagtccgtccaactgaaagaaaagagaggagtggtgct | |
| | cctgtacatcctgcttggcaccatcgggaccctggtggccgtgctggcggcgagtgccctcctctacaggcactggatt | |
| | gaaatagtgctgctgtaccggacctaccagagcaaggatcagacgcttggggataaaaaggattttgatgctttcgtatc | |
| | ctatgcaaaatggagctcttttccaagtgaggccacttcatctctgagtgaagaacacttggccctgagcctatttcctgat | |
| | gttttagaaaacaaatatggatatagcctgtgtttgcttgaaagagatgtggctccaggaggagtgtatgcagaagacatt | |
| | gtgagcattattaagagaagcagaagaggaatatttatcttgagccccaactatgtcaatggacccagtatctttgaacta | |
| | caagcagcagtgaatcttgccttggatgatcaaacactgaaactcattttaattaagttctgttacttccaagagccagagt | |
| | ctctacctcatctcgtgaaaaaagctctcagggttttgcccacagttacttggagaggcttaaaatcagttcctcccaattct | |
| | aggttctgggccaaaatgcgctaccacatgcctgtgaaaaactctcagggattcacgtggaaccagctcagaattacct | |
| | ctaggatttttcagtggaaaggactcagtagaacagaaaccactgggaggagctcccagcctaaggaatggtgataa | |
| CAR 20A10N | atggccttaccagtgaccgccttgctcctgccgctggccttgctgctccacgccgccaggccgcaggtgcagctggtt | 167 |
| CD28-CD28-CD28 | gaatctggcggcggacttgtgaagcctggcggatctctgagactgagctgtgccgccagcggcttcacctttagcacat | |
| 1XX3XIL2NFAT | acgccatgagctggatcagacaggcccctggcaaaggcctggaatgggtggcgtctattggcagagccggcagcac | |
| mCMV | ctactacagcgactctgtgaagggcagattcaccatcagccgggacaacgccaagaacagcctgtacctgcagatga | |
| IL18Ralpha-T2A- | actccctgagagccgaggacaccgccgtgtactattgtgccagaggacccatctacaacgactacgacgagttcgcct | |
| IL18Rb | attggggccagggcacactggtcacagtttctagcggcggtggggaagcggaggcggtggctccggtggcggag | |
| | gcagcgaaattgtgctgacacagagccccgccacactgtcactttctccaggcgaaagagccacactgagctgcaag | |
| | agcagccagagcgtgtgtactccagcaaccagaagaactacctggcctggtatcagcagaagcccggccaagctc | |
| | ctcggctgctgatctattgggccagcacaagagagagcggcatccctgccagattttctggcagcggctctggcaccg | |
| | atttcaccctgaccataagcagcctggaacctgaggacttcgccgtgtattactgccaccagtacctgagcagcctgac | |
| | ctttggcggaggcaccaaggtggaaatcaagcggaagcacctgtgtccttctccactgttccccggccctagcaagcc | |
| | tttctgggtcctggtggtggtgggcggagtgctggcctgctacagcctgctggtgaccgtggcctttatcatcttctgggt | |
| | gaggagtaagaggagcaggctcctgcacagtgactacatgaacatgactcctagaagacctgggcctaccagaaag | |
| | cattaccagccctatgccccaccacgcgacttcgcagcctatcgctccagagtgaagttcagcaggagcgcagacgc | |
| | ccccgcgtacaagcagggccagaaccagctctataacgagctcaatctaggacgaagagaggagtacgatgttttgg | |
| | acaagagacgtggccgggaccctgagatggggggaaagccgagaaggaagaaccctcaggaaggcctgttcaat | |
| | gaactgcagaaagataagatggcggaggccttcagtgagattgggatgaaaggcgagcgccggaggggcaaggg | |
| | gcacgatggccttttccagggtctcagtacagccaccaaggacaccttcgacgcccttcacatgcaggccctgccccc | |
| | tcgctgataagtttaaactgccagaacattactctggcctaactggccggtaccggaggaaaaactgtttcatacagaag | |
| | gcgtggaggaaaaactgtttcatacagaaggcgtggaggaaaaactgtttcatacagaaggcgtactagttaggcgtgt | |
| | acggtgggaggcctatataagcagagctcgtttagtgaaccgtcagatcgcctggagacgccatccacgctgttttgac | |
| | ctccatagaagacaccgggaccgatccagcctcgagagacccaatgctagccaccatgaattgtagagaattaccctt | |
| | gaccctttgggtgcttatatctgtaagcactgcagaatcttgtacttcacgtccccacattactgtggttgaaggggaacct | |
| | ttctatctgaaacattgctcgtgttcacttgcacatgagattgaaacaaccaccaaaagctggtacaaaagcagtggatca | |
| | caggaacatgtggagctgaacccaaggagttccagcagaattgctttgcatgattgtgttttggagttttggccagttgag | |
| | ttgaatgacacaggatcttactttttccaaatgaaaaattatactcagaaatggaaattaaatgtcatcagaagaaataaac | |
| | acagctgtttcactgaaagacaagtaactagtaaaattgtggaagttaaaaaattttttcagataacctgtgaaaacagtta | |
| | ctatcaaacactggtcaacagcacatcattgtataagaactgtaaaaagctactactggagaacaataaaaacccaacg | |
| | ataaagaagaacgccgagtttgaagatcaggggtattactcctgcgtgcatttccttcatcataatggaaaactatttaata | |
| | tcaccaaaaccttcaatataacaatagtggaagatcgcagtaatatagttccggttcttcttggaccaaagcttaaccatgt | |
| | tgcagtggaattaggaaaaaacgtaaggctcaactgctctgctttgctgaatgaagaggatgtaatttattggatgttcgg | |
| | ggaagaaaatggatcggatcctaatatacatgaagagaaagaaatgagaattatgactccagaaggcaaatggcatgc | |
| | ttcaaaagtattgagaattgaaaatattggtgaaagcaatctaaatgttttatataattgcactgtggccagcacgggaggc | |
| | acagacaccaaaagcttcatcttggtgagaaaagcagacatggctgatatcccaggccacgtcttcacaagaggaatg | |
| | atcatagctgttttgatcttggtggcagtagtgtgcctagtgactgtgtgtgtcatttatagagttgacttggttctattttatag | |
| | acatttaacgagaagagatgaaacattaacagatggaaaaacatatgatgcttttgtgtcttacctaaaagaatgccgacc | |
| | tgaaaatggagaggagcacacctttgctgtggagattttgcccagggtgttggagaaacattttgggtataagttatgcat | |
| | atttgaaagggatgtagtgcctggaggagctgttgttgatgaaatccactcactgatagagaaaagccgaagactaatc | |
| | attgtcctaagtaaaagttatatgtctaatgaggtcaggtatgaacttgaaagtggactccatgaagcattggtggaaaga | |
| | aaaattaaaataatcttaattgaatttacacctgttactgacttcacattcttgccccaatcactaaagcttttgaaatctcaca | |
| | gagttctgaagtggaaggccgataaatctctttcttataactcaaggttctggaagaaccttctttacttaatgcctgcaaaa | |
| | acagtcaagccaggtagagacgaaccggaagtcttgcctgttctttccgagtctggaagcggtgagggaagaggtag | |
| | cctgctcacgtgcggtgatgtggaggagaaccctgggcctatgctctgtttgggctggatatttctttggcttgttgcagg | |
| | agagcgaattaaaggatttaatatttcaggttgttccacaaaaaaactcctttggacatattctacaaggagtgaagagga | |
| | atttgtcttattttgtgatttaccagagccacagaaatcacatttctgccacagaaatcgactctcaccaaaacaagtccctg | |
| | agcacctgcccttcatgggtagtaacgacctatctgatgtccaatggtaccaacaaccttcgaatggagatccattagag | |
| | gacattaggaaaagctatcctcacatcattcaggacaaatgtacccttcactttttgaccccaggggtgaataattctgggt | |
| | catatatttgtagacccaagatgattaagagcccctatgatgtagcctgttgtgtcaagatgattttagaagttaagcccca | |
| | gacaaatgcatcctgtgagtattccgcatcacataagcaagacctacttcttgggagcactggctctatttcttgccccagt | |
| | ctcagctgccaaagtgatgcacaaagtccagcggtaacctggtacaagaatggaaaactcctctctgtggaaaggagc | |
| | aaccgaatcgtagtggatgaagtttatgactatcaccagggcacatatgtatgtgattacactcagtcggatactgtgagt | |
| | tcgtggacagtcagagctgttgttcaagtgagaaccattgtgggagacactaaactcaaaccagatattctggatcctgt | |
| | cgaggacacactggaagtagaacttggaaagcctttaactattagctgcaaagcacgatttggctttgaaagggtcttta | |
| | accctgtcataaaatggtacatcaaagattctgacctagagtgggaagtctcagtacctgaggcgaaaagtattaaatcc | |
| | actttaaaggatgaaatcattgagcgtaatatcatcttggaaaaagtcactcagcgtgatcttcgcaggaagtttgtttgctt | |
| | tgtccagaactccattggaaacacaacccagtccgtccaactgaaagaaaagagaggagtggtgctcctgtacatcct | |
| | gcttggcaccatcgggaccctggtggccgtgctggcggcgagtgccctcctctacaggcactggattgaaatagtgct | |
| | gctgtaccggacctaccagagcaaggatcagacgcttggggataaaaaggattttgatgctttcgtatcctatgcaaaat | |
| | ggagctcttttccaagtgaggccacttcatctctgagtgaagaacacttggccctgagcctatttcctgatgttttagaaaa | |
| | caaatatggatatagcctgtgtttgcttgaaagagatgtggctccaggaggagtgtatgcagaagacattgtgagcatta | |
| | ttaagagaagcagaagaggaatatttatcttgagccccaactatgtcaatggacccagtatctttgaactacaagcagca | |
| | gtgaatcttgccttggatgatcaaacactgaaactcattttaattaagttctgttacttccaagagccagagtctctacctcat | |
| | ctcgtgaaaaaagctctcagggttttgcccacagttacttggagaggcttaaaatcagttcctcccaattctaggttctggg | |
| | ccaaaatgcgctaccacatgcctgtgaaaaactctcagggattcacgtggaaccagctcagaattacctctaggatttttc | |
| | agtggaaaggactcagtagaacagaaaccactgggaggagctcccagcctaaggaatggtgataa | |
| CAR C2 CD8- | atggccttaccagtgaccgccttgctcctgccgctggccttgctgctccacgccgccaggccggaggtgcagctggtg | 168 |
| CD8-41BB1XX | gagtctgggggaggcctggtcaagcctggggggtccctgagactctcctgtgcagcctctggattcaccttcagtggc | |
| 6XFOXP3NFAT | tatgccatgagctgggtccgccaggctccagggaaggggctggagtgggtctcaaccattagtagtggcggaaccta | |
| mCMV | catatactaccccgactcagtgaagggccgattcaccatctccagagacaacgccaagaactcactgtatctgcaaatg | |
| IL 18Ralpha | aacagcctgagagccgaggacacggccgtgtattactgtgcgagacttgggggggataattactacgaatacttcgat | |
| | gtctggggcaaagggaccacggtcaccgtctcctccggcggtggcggatccggcggtggcggatccggcggtggc | |
| | ggatccgacattgtgctgacccagtctccagcctccttggccgtgtctccaggacagagggccaccatcacctgcaga | |
| | gccagtaagagtgtcagtaccagcggatactcctacatgcactggtatcagcagaaaccaggacaacctcctaaactc | |
| | ctgatttacctggcatccaatctggagagcggggtcccagccaggttcagcggcagtgggtctgggaccgatttcacc | |
| | ctcacaattaatcctgtggaagctaatgatactgcaaattattactgtcagcacagtagggagctgcctttcacattcggc | |
| | ggagggaccaaggtggagatcaaacgaactacaacaacccctgcccccagacctcctaccccagcccctacaattg | |
| | ccagccagcctctgagcctgaggcccgaggcttgtagacctgctgctggcggagccgtgcacaccagaggactgga | |
| | tttcgcctgcgacatctacatctgggcgcccttggccgggacttgtggggtccttctcctgtcactggttatcaccctttact | |
| | gcaaacggggcagaaagaaactcctgtatatattcaaacaaccatttatgagaccagtacaaactactcaagaggaag | |
| | atggctgtagctgccgatttccagaagaagaagaaggaggatgtgaactgagagtgaagttcagcaggagcgcaga | |
| | cgcccccgcgtacaagcagggccagaaccagctctataacgagctcaatctaggacgaagagaggagtacgatgttt | |
| | tggacaagagacgtggccgggaccctgagatggggggaaagccgagaaggaagaaccctcaggaaggcctgttc | |
| | aatgaactgcagaaagataagatggcggaggccttcagtgagattgggatgaaaggcgagcgccggaggggcaag | |
| | gggcacgatggccttttccagggtctcagtacagccaccaaggacaccttcgacgcccttcacatgcaggccctgccc | |
| | cctcgc | |
| | tgataagtttaaactgccagaacatttctctggcctaactggccggtaccggcttcattttttccatttactgcagaggcttc | |
| | attttttccatttactgcagaggcttcattttttccatttactgcagaggcttcattttttccatttactgcagaggcttcattttttc | |
| | catttactgcagaggcttcattttttccatttactgcagaactagttaggcgtgtacggtgggaggcctatataagcagagc | |
| | tcgtttagtgaaccgtcagatcgcctggagacgccatccacgctgttttgacctccatagaagacaccgggaccgatcc | |
| | agcctcgagagacccaatgctagccaccatgaattgtagagaattacccttgaccctttgggtgcttatatctgtaagca | |
| | ctgcagaatcttgtacttcacgtccccacattactgtggttgaaggggaacctttctatctgaaacattgctcgtgttcactt | |
| | gcacatgagattgaaacaaccaccaaaagctggtacaaaagcagtggatcacaggaacatgtggagctgaacccaa | |
| | ggagttccagcagaattgctttgcatgattgtgttttggagttttggccagttgagttgaatgacacaggatcttactttttcc | |
| | aaatgaaaaattatactcagaaatggaaattaaatgtcatcagaagaaataaacacagctgtttcactgaaagacaagta | |
| | actagtaaaattgtggaagttaaaaaattttttcagataacctgtgaaaacagttactatcaaacactggtcaacagcacat | |
| | cattgtataagaactgtaaaaagctactactggagaacaataaaaacccaacgataaagaagaacgccgagtttgaag | |
| | atcaggggtattactcctgcgtgcatttccttcatcataatggaaaactatttaatatcaccaaaaccttcaatataacaata | |
| | gtggaagatcgcagtaatatagttccggttcttcttggaccaaagcttaaccatgttgcagtggaattaggaaaaaacgta | |
| | aggctcaactgctctgctttgctgaatgaagaggatgtaatttattggatgttcggggaagaaaatggatcggatcctaat | |
| | atacatgaagagaaagaaatgagaattatgactccagaaggcaaatggcatgcttcaaaagtattgagaattgaaaatat | |
| | tggtgaaagcaatctaaatgttttatataattgcactgtggccagcacgggaggcacagacaccaaaagcttcatcttgg | |
| | tgagaaaagcagacatggctgatatcccaggccacgtcttcacaagaggaatgatcatagctgttttgatcttggtggca | |
| | gtagtgtgcctagtgactgtgtgtgtcatttatagagttgacttggttctattttatagacatttaacgagaagagatgaaac | |
| | attaacagatggaaaaacatatgatgcttttgtgtcttacctaaaagaatgccgacctgaaaatggagaggagcacacct | |
| | ttgctgtggagattttgcccagggtgttggagaaacattttgggtataagttatgcatatttgaaagggatgtagtgcctgg | |
| | aggagctgttgttgatgaaatccactcactgatagagaaaagccgaagactaatcattgtcctaagtaaaagttatatgtc | |
| | taatgaggtcaggtatgaacttgaaagtggactccatgaagcattggtggaaagaaaaattaaaataatcttaattgaattt | |
| | acacctgttactgacttcacattcttgccccaatcactaaagcttttgaaatctcacagagttctgaagtggaaggccgata | |
| | aatctctttcttataactcaaggttctggaagaaccttctttacttaatgcctgcaaaaacagtcaagccaggtagagacga | |
| | accggaagtcttgcctgttctttccgagtcttgataa | |
| CAR C2 CD8- | atggccttaccagtgaccgccttgctcctgccgctggccttgctgctccacgccgccaggccggaggtgcagctggtg | 169 |
| CD8-41BB1XX | gagtctgggggaggcctggtcaagcctggggggtccctgagactctcctgtgcagcctctggattcaccttcagtggc | |
| 3XFOXP3NFAT | tatgccatgagctgggtccgccaggctccagggaaggggctggagtgggtctcaaccattagtagtggcggaaccta | |
| mCMV | catatactaccccgactcagtgaagggccgattcaccatctccagagacaacgccaagaactcactgtatctgcaaatg | |
| IL18Ralpha | aacagcctgagagccgaggacacggccgtgtattactgtgcgagacttgggggggataattactacgaatacttcgat | |
| | gtctggggcaaagggaccacggtcaccgtctcctccggcggtggcggatccggcggtggcggatccggcggtggc | |
| | ggatccgacattgtgctgacccagtctccagcctccttggccgtgtctccaggacagagggccaccatcacctgcaga | |
| | gccagtaagagtgtcagtaccagcggatactcctacatgcactggtatcagcagaaaccaggacaacctcctaaactc | |
| | ctgatttacctggcatccaatctggagagcggggtcccagccaggttcagcggcagtgggtctgggaccgatttcacc | |
| | ctcacaattaatcctgtggaagctaatgatactgcaaattattactgtcagcacagtagggagctgcctttcacattcggc | |
| | ggagggaccaaggtggagatcaaacgaactacaacaacccctgcccccagacctcctaccccagcccctacaattg | |
| | ccagccagcctctgagcctgaggcccgaggcttgtagacctgctgctggcggagccgtgcacaccagaggactgga | |
| | tttcgcctgcgacatctacatctgggcgcccttggccgggacttgtggggtccttctcctgtcactggttatcaccctttact | |
| | gcaaacggggcagaaagaaactcctgtatatattcaaacaaccatttatgagaccagtacaaactactcaagaggaag | |
| | atggctgtagctgccgatttccagaagaagaagaaggaggatgtgaactgagagtgaagttcagcaggagcgcaga | |
| | cgcccccgcgtacaagcagggccagaaccagctctataacgagctcaatctaggacgaagagaggagtacgatgttt | |
| | tggacaagagacgtggccgggaccctgagatggggggaaagccgagaaggaagaaccctcaggaaggcctgttc | |
| | aatgaactgcagaaagataagatggcggaggccttcagtgagattgggatgaaaggcgagcgccggaggggcaag | |
| | gggcacgatggccttttccagggtctcagtacagccaccaaggacaccttcgacgcccttcacatgcaggccctgccc | |
| | cctcgctgataagtttaaactgccagaacatttctctggcctaactggccggtaccggcttcattttttccatttactgcaga | |
| | ggcttcattttttccatttactgcagaggcttcattttttccatttactgcagaactagttaggcgtgtacggtgggaggccta | |
| | tataagcagagctcgtttagtgaaccgtcagatcgcctggagacgccatccacgctgttttgacctccatagaagacac | |
| | cgggaccgatccagcctcgagagacccaatgctagccaccatgaattgtagagaattacccttgaccctttgggtgctt | |
| | atatctgtaagcactgcagaatcttgtacttcacgtccccacattactgtggttgaaggggaacctttctatctgaaacattg | |
| | ctcgtgttcacttgcacatgagattgaaacaaccaccaaaagctggtacaaaagcagtggatcacaggaacatgtgga | |
| | gctgaacccaaggagttccagcagaattgctttgcatgattgtgttttggagttttggccagttgagttgaatgacacagg | |
| | atcttactttttccaaatgaaaaattatactcagaaatggaaattaaatgtcatcagaagaaataaacacagctgtttcactg | |
| | aaagacaagtaactagtaaaattgtggaagttaaaaaattttttcagataacctgtgaaaacagttactatcaaacactggt | |
| | caacagcacatcattgtataagaactgtaaaaagctactactggagaacaataaaaacccaacgataaagaagaacgc | |
| | cgagtttgaagatcaggggtattactcctgcgtgcatttccttcatcataatggaaaactatttaatatcaccaaaaccttca | |
| | atataacaatagtggaagatcgcagtaatatagttccggttcttcttggaccaaagcttaaccatgttgcagtggaattagg | |
| | aaaaaacgtaaggctcaactgctctgctttgctgaatgaagaggatgtaatttattggatgttcggggaagaaaatggat | |
| | cggatcctaatatacatgaagagaaagaaatgagaattatgactccagaaggcaaatggcatgcttcaaaagtattgag | |
| | aattgaaaatattggtgaaagcaatctaaatgttttatataattgcactgtggccagcacgggaggcacagacaccaaaa | |
| | gcttcatcttggtgagaaaagcagacatggctgatatcccaggccacgtcttcacaagaggaatgatcatagctgttttg | |
| | atcttggtggcagtagtgtgcctagtgactgtgtgtgtcatttatagagttgacttggttctattttatagacatttaacgaga | |
| | agagatgaaacattaacagatggaaaaacatatgatgcttttgtgtcttacctaaaagaatgccgacctgaaaatggaga | |
| | ggagcacacctttgctgtggagattttgcccagggtgttggagaaacattttgggtataagttatgcatatttgaaaggga | |
| | tgtagtgcctggaggagctgttgttgatgaaatccactcactgatagagaaaagccgaagactaatcattgtcctaagta | |
| | aaagttatatgtctaatgaggtcaggtatgaacttgaaagtggactccatgaagcattggtggaaagaaaaattaaaata | |
| | atcttaattgaatttacacctgttactgacttcacattcttgccccaatcactaaagcttttgaaatctcacagagttctgaagt | |
| | ggaaggccgataaatctctttcttataactcaaggttctggaagaaccttctttacttaatgcctgcaaaaacagtcaagcc | |
| | aggtagagacgaaccggaagtcttgcctgttctttccgagtcttgataa | |
| CAR C2 CD8- | atggccttaccagtgaccgccttgctcctgccgctggccttgctgctccacgccgccaggccggaggtgcagctggtg | 170 |
| CD8-41BB1XX | gagtctgggggaggcctggtcaagcctggggggtccctgagactctcctgtgcagcctctggattcaccttcagtggc | |
| 6XIL12NFAT | tatgccatgagctgggtccgccaggctccagggaaggggctggagtgggtctcaaccattagtagtggcggaaccta | |
| mCMV | catatactaccccgactcagtgaagggccgattcaccatctccagagacaacgccaagaactcactgtatctgcaaatg | |
| IL18Ralpha | aacagcctgagagccgaggacacggccgtgtattactgtgcgagacttgggggggataattactacgaatacttcgat | |
| | gtctggggcaaagggaccacggtcaccgtctcctccggcggtggcggatccggcggtggcggatccggcggtggc | |
| | ggatccgacattgtgctgacccagtctccagcctccttggccgtgtctccaggacagagggccaccatcacctgcaga | |
| | gccagtaagagtgtcagtaccagcggatactcctacatgcactggtatcagcagaaaccaggacaacctcctaaactc | |
| | ctgatttacctggcatccaatctggagagcggggtcccagccaggttcagcggcagtgggtctgggaccgatttcacc | |
| | ctcacaattaatcctgtggaagctaatgatactgcaaattattactgtcagcacagtagggagctgcctttcacattcggc | |
| | ggagggaccaaggtggagatcaaacgaactacaacaacccctgcccccagacctcctaccccagcccctacaattg | |
| | ccagccagcctctgagcctgaggcccgaggcttgtagacctgctgctggcggagccgtgcacaccagaggactgga | |
| | tttcgcctgcgacatctacatctgggcgcccttggccgggacttgtggggtccttctcctgtcactggttatcaccctttact | |
| | gcaaacggggcagaaagaaactcctgtatatattcaaacaaccatttatgagaccagtacaaactactcaagaggaag | |
| | atggctgtagctgccgatttccagaagaagaagaaggaggatgtgaactgagagtgaagttcagcaggagcgcaga | |
| | cgcccccgcgtacaagcagggccagaaccagctctataacgagctcaatctaggacgaagagaggagtacgatgttt | |
| | tggacaagagacgtggccgggaccctgagatggggggaaagccgagaaggaagaaccctcaggaaggcctgttc | |
| | aatgaactgcagaaagataagatggcggaggccttcagtgagattgggatgaaaggcgagcgccggaggggcaag | |
| | gggcacgatggccttttccagggtctcagtacagccaccaaggacaccttcgacgcccttcacatgcaggccctgccc | |
| | cctcgctgataagtttaaactgccagaacatttctctggcctaactggccggtaccggaggaaaaactgtttcatacaga | |
| | aggcgtggaggaaaaactgtttcatacagaaggcgtggaggaaaaactgtttcatacagaaggcgtggaggaaaaac | |
| | tgtttcatacagaaggcgtggaggaaaaactgtttcatacagaaggcgtggaggaaaaactgtttcatacagaaggcgt | |
| | actagttaggcgtgtacggtgggaggcctatataagcagagctcgtttagtgaaccgtcagatcgcctggagacgccat | |
| | ccacgctgttttgacctccatagaagacaccgggaccgatccagcctcgagagacccaatgctagccaccatgaatt | |
| | gtagagaattacccttgaccctttgggtgcttatatctgtaagcactgcagaatcttgtacttcacgtccccacattactgtg | |
| | gttgaaggggaacctttctatctgaaacattgctcgtgttcacttgcacatgagattgaaacaaccaccaaaagctggtac | |
| | aaaagcagtggatcacaggaacatgtggagctgaacccaaggagttccagcagaattgctttgcatgattgtgttttgga | |
| | gttttggccagttgagttgaatgacacaggatcttactttttccaaatgaaaaattatactcagaaatggaaattaaatgtcat | |
| | cagaagaaataaacacagctgtttcactgaaagacaagtaactagtaaaattgtggaagttaaaaaattttttcagataac | |
| | ctgtgaaaacagttactatcaaacactggtcaacagcacatcattgtataagaactgtaaaaagctactactggagaaca | |
| | ataaaaacccaacgataaagaagaacgccgagtttgaagatcaggggtattactcctgcgtgcatttccttcatcataat | |
| | ggaaaactatttaatatcaccaaaaccttcaatataacaatagtggaagatcgcagtaatatagttccggttcttcttggac | |
| | caaagcttaaccatgttgcagtggaattaggaaaaaacgtaaggctcaactgctctgctttgctgaatgaagaggatgta | |
| | atttattggatgttcggggaagaaaatggatcggatcctaatatacatgaagagaaagaaatgagaattatgactccaga | |
| | aggcaaatggcatgcttcaaaagtattgagaattgaaaatattggtgaaagcaatctaaatgttttatataattgcactgtg | |
| | gccagcacgggaggcacagacaccaaaagcttcatcttggtgagaaaagcagacatggctgatatcccaggccacg | |
| | tcttcacaagaggaatgatcatagctgttttgatcttggtggcagtagtgtgcctagtgactgtgtgtgtcatttatagagtt | |
| | gacttggttctattttatagacatttaacgagaagagatgaaacattaacagatggaaaaacatatgatgcttttgtgtctta | |
| | cctaaaagaatgccgacctgaaaatggagaggagcacacctttgctgtggagattttgcccagggtgttggagaaaca | |
| | ttttgggtataagttatgcatatttgaaagggatgtagtgcctggaggagctgttgttgatgaaatccactcactgatagag | |
| | aaaagccgaagactaatcattgtcctaagtaaaagttatatgtctaatgaggtcaggtatgaacttgaaagtggactccat | |
| | gaagcattggtggaaagaaaaattaaaataatcttaattgaatttacacctgttactgacttcacattcttgccccaatcact | |
| | aaagcttttgaaatctcacagagttctgaagtggaaggccgataaatctctttcttataactcaaggttctggaagaacctt | |
| | ctttacttaatgcctgcaaaaacagtcaagccaggtagagacgaaccggaagtcttgcctgttctttccgagtcttgataa | |
| CAR C2 CD8- | atggccttaccagtgaccgccttgctcctgccgctggccttgctgctccacgccgccaggccggaggtgcagctggtg | 171 |
| CD8-41BB1XX | gagtctgggggaggcctggtcaagcctggggggtccctgagactctcctgtgcagcctctggattcaccttcagtggc | |
| 3XIL12NFAT | tatgccatgagctgggtccgccaggctccagggaaggggctggagtgggtctcaaccattagtagtggcggaaccta | |
| mCMV | catatactaccccgactcagtgaagggccgattcaccatctccagagacaacgccaagaactcactgtatctgcaaatg | |
| IL18Ralpha | aacagcctgagagccgaggacacggccgtgtattactgtgcgagacttgggggggataattactacgaatacttcgat | |
| | gtctggggcaaagggaccacggtcaccgtctcctccggcggtggcggatccggcggtggggatccggcggtggc | |
| | ggatccgacattgtgctgacccagtctccagcctccttggccgtgtctccaggacagagggccaccatcacctgcaga | |
| | gccagtaagagtgtcagtaccagcggatactcctacatgcactggtatcagcagaaaccaggacaacctcctaaactc | |
| | ctgatttacctggcatccaatctggagagcggggtcccagccaggttcagcggcagtgggtctgggaccgatttcacc | |
| | ctcacaattaatcctgtggaagctaatgatactgcaaattattactgtcagcacagtagggagctgcctttcacattcggc | |
| | ggagggaccaaggtggagatcaaacgaactacaacaacccctgcccccagacctcctaccccagcccctacaattg | |
| | ccagccagcctctgagcctgaggcccgaggcttgtagacctgctgctggcggagccgtgcacaccagaggactgga | |
| | tttcgcctgcgacatctacatctgggcgcccttggccgggacttgtggggtccttctcctgtcactggttatcaccctttact | |
| | gcaaacggggcagaaagaaactcctgtatatattcaaacaaccatttatgagaccagtacaaactactcaagaggaag | |
| | atggctgtagctgccgatttccagaagaagaagaaggaggatgtgaactgagagtgaagttcagcaggagcgcaga | |
| | cgcccccgcgtacaagcagggccagaaccagctctataacgagctcaatctaggacgaagagaggagtacgatgttt | |
| | tggacaagagacgtggccgggaccctgagatggggggaaagccgagaaggaagaaccctcaggaaggcctgttc | |
| | aatgaactgcagaaagataagatggcggaggccttcagtgagattgggatgaaaggcgagcgccggaggggcaag | |
| | gggcacgatggccttttccagggtctcagtacagccaccaaggacaccttcgacgcccttcacatgcaggccctgccc | |
| | cctcgctgataagtttaaactgccagaacatttctctggcctaactggccggtaccggaggaaaaactgtttcatacaga | |
| | aggcgtggaggaaaaactgtttcatacagaaggcgtggaggaaaaactgtttcatacagaaggcgtactagttaggcg | |
| | tgtacggtgggaggcctatataagcagagctcgtttagtgaaccgtcagatcgcctggagacgccatccacgctgtttt | |
| | gacctccatagaagacaccgggaccgatccagcctcgagagacccaatgctagccaccatgaattgtagagaattac | |
| | ccttgaccctttgggtgcttatatctgtaagcactgcagaatcttgtacttcacgtccccacattactgtggttgaagggga | |
| | acctttctatctgaaacattgctcgtgttcacttgcacatgagattgaaacaaccaccaaaagctggtacaaaagcagtgg | |
| | atcacaggaacatgtggagctgaacccaaggagttccagcagaattgctttgcatgattgtgttttggagttttggccagt | |
| | tgagttgaatgacacaggatcttactttttccaaatgaaaaattatactcagaaatggaaattaaatgtcatcagaagaaat | |
| | aaacacagctgtttcactgaaagacaagtaactagtaaaattgtggaagttaaaaaattttttcagataacctgtgaaaaca | |
| | gttactatcaaacactggtcaacagcacatcattgtataagaactgtaaaaagctactactggagaacaataaaaaccca | |
| | acgataaagaagaacgccgagtttgaagatcaggggtattactcctgcgtgcatttccttcatcataatggaaaactattt | |
| | aatatcaccaaaaccttcaatataacaatagtggaagatcgcagtaatatagttccggttcttcttggaccaaagcttaacc | |
| | atgttgcagtggaattaggaaaaaacgtaaggctcaactgctctgctttgctgaatgaagaggatgtaatttattggatgtt | |
| | cggggaagaaaatggatcggatcctaatatacatgaagagaaagaaatgagaattatgactccagaaggcaaatggc | |
| | atgcttcaaaagtattgagaattgaaaatattggtgaaagcaatctaaatgttttatataattgcactgtggccagcacggg | |
| | aggcacagacaccaaaagcttcatcttggtgagaaaagcagacatggctgatatcccaggccacgtcttcacaagag | |
| | gaatgatcatagctgttttgatcttggtggcagtagtgtgcctagtgactgtgtgtgtcatttatagagttgacttggttctatt | |
| | ttatagacatttaacgagaagagatgaaacattaacagatggaaaaacatatgatgcttttgtgtcttacctaaaagaatgc | |
| | cgacctgaaaatggagaggagcacacctttgctgtggagattttgcccagggtgttggagaaacattttgggtataagtt | |
| | atgcatatttgaaagggatgtagtgcctggaggagctgttgttgatgaaatccactcactgatagagaaaagccgaaga | |
| | ctaatcattgtcctaagtaaaagttatatgtctaatgaggtcaggtatgaacttgaaagtggactccatgaagcattggtgg | |
| | aaagaaaaattaaaataatcttaattgaatttacacctgttactgacttcacattcttgccccaatcactaaagcttttgaaatc | |
| | tcacagagttctgaagtggaaggccgataaatctctttcttataactcaaggttctggaagaaccttctttacttaatgcctg | |
| | caaaaacagtcaagccaggtagagacgaaccggaagtcttgcctgttctttccgagtcttgataa | |
| CAR E6 CD8- | atggccttaccagtgaccgccttgctcctgccgctggccttgctgctccacgccgccaggccggaggtgcagctggtg | 172 |
| CD8-41BB1XX | gagtctgggggaggcctggtcaagcctggggggtccctgagactctcctgtgcagcctctggattcaccttcagtagg | |
| 6XFOXP3NFAT | tatggcatgagctgggtccgccaggctccagggaagaggctggagtgggtctcaaccattagtggcggaggcacct | |
| mCMV | acatatactacccagactcagtgaagggccgattcaccatctccagagacaacgccaagaacaccctgtatctgcaaat | |
| IL18Ralpha | gaacagcctgagagccgaggacacggctgtgtattactgtaccagagataactatggccgcaactatgattatggcatg | |
| | gattattggggccagggcaccctggtgaccgtgagcagcggtggtggcggttccggcggtggcggttccggtggtg | |
| | gcggttccgaaattgtgttgacacagtctccagccaccctgtctttgtctccaggggaaagagccaccctcacctgcag | |
| | cgccaccagcagtgttagctacatccactggtatcaacagaggcctggccagagccccaggctcctcatctatagcac | |
| | ctccaacctggccagcggcatcccagccaggttcagtggcagtgggtctgggagcgactacactctcaccatcagca | |
| | gcctagagcctgaagattttgcagtttattactgtcagcagcgtagcagctcccctttcacctttggcagcggcaccaaa | |
| | gtggaaattaaaacaacaacccctgcccccagacctcctaccccagcccctacaattgccagccagcctctgagcctg | |
| | aggcccgaggcttgtagacctgctgctggcggagccgtgcacaccagaggactggatttcgcctgcgacatctacatc | |
| | tgggcgcccttggccgggacttgtggggtccttctcctgtcactggttatcaccctttactgcaaacggggcagaaaga | |
| | aactcctgtatatattcaaacaaccatttatgagaccagtacaaactactcaagaggaagatggctgtagctgccgatttc | |
| | cagaagaagaagaaggaggatgtgaactgagagtgaagttcagcaggagcgcagacgcccccgcgtacaagcag | |
| | ggccagaaccagctctataacgagctcaatctaggacgaagagaggagtacgatgttttggacaagagacgtggccg | |
| | ggaccctgagatggggggaaagccgagaaggaagaaccctcaggaaggcctgttcaatgaactgcagaaagataa | |
| | gatggcggaggccttcagtgagattgggatgaaaggcgagcgccggaggggcaaggggcacgatggccttttcca | |
| | gggtctcagtacagccaccaaggacaccttcgacgcccttcacatgcaggccctgccccctcgctgataagtttaaact | |
| | gccagaacatttctctggcctaactggccggtaccggcttcattttttccatttactgcagaggcttcattttttccatttactg | |
| | cagaggcttcattttttccatttactgcagaggcttcattttttccatttactgcagaggcttcattttttccatttactgcagag | |
| | gcttcattttttccatttactgcagaactagttaggcgtgtacggtgggaggcctatataagcagagctcgtttagtgaacc | |
| | gtcagatcgcctggagacgccatccacgctgttttgacctccatagaagacaccgggaccgatccagcctcgagaga | |
| | cccaatgctagccaccatgaattgtagagaattacccttgaccctttgggtgcttatatctgtaagcactgcagaatcttgt | |
| | acttcacgtccccacattactgtggttgaaggggaacctttctatctgaaacattgctcgtgttcacttgcacatgagattga | |
| | aacaaccaccaaaagctggtacaaaagcagtggatcacaggaacatgtggagctgaacccaaggagttccagcaga | |
| | attgctttgcatgattgtgttttggagttttggccagttgagttgaatgacacaggatcttactttttccaaatgaaaaattata | |
| | ctcagaaatggaaattaaatgtcatcagaagaaataaacacagctgtttcactgaaagacaagtaactagtaaaattgtg | |
| | gaagttaaaaaattttttcagataacctgtgaaaacagttactatcaaacactggtcaacagcacatcattgtataagaact | |
| | gtaaaaagctactactggagaacaataaaaacccaacgataaagaagaacgccgagtttgaagatcaggggtattact | |
| | cctgcgtgcatttccttcatcataatggaaaactatttaatatcaccaaaaccttcaatataacaatagtggaagatcgcagt | |
| | aatatagttccggttcttcttggaccaaagcttaaccatgttgcagtggaattaggaaaaaacgtaaggctcaactgctct | |
| | gctttgctgaatgaagaggatgtaatttattggatgttcggggaagaaaatggatcggatcctaatatacatgaagagaa | |
| | agaaatgagaattatgactccagaaggcaaatggcatgcttcaaaagtattgagaattgaaaatattggtgaaagcaatc | |
| | taaatgttttatataattgcactgtggccagcacgggaggcacagacaccaaaagcttcatcttggtgagaaaagcaga | |
| | catggctgatatcccaggccacgtcttcacaagaggaatgatcatagctgttttgatcttggtggcagtagtgtgcctagt | |
| | gactgtgtgtgtcatttatagagttgacttggttctattttatagacatttaacgagaagagatgaaacattaacagatggaa | |
| | aaacatatgatgcttttgtgtcttacctaaaagaatgccgacctgaaaatggagaggagcacacctttgctgtggagatttt | |
| | gcccagggtgttggagaaacattttgggtataagttatgcatatttgaaagggatgtagtgcctggaggagctgttgttga | |
| | tgaaatccactcactgatagagaaaagccgaagactaatcattgtcctaagtaaaagttatatgtctaatgaggtcaggta | |
| | tgaacttgaaagtggactccatgaagcattggtggaaagaaaaattaaaataatcttaattgaatttacacctgttactgac | |
| | ttcacattcttgccccaatcactaaagcttttgaaatctcacagagttctgaagtggaaggccgataaatctctttcttataac | |
| | tcaaggttctggaagaaccttctttacttaatgcctgcaaaaacagtcaagccaggtagagacgaaccggaagtcttgc | |
| | ctgttctttccgagtcttgataa | |
| CAR E6 CD8- | atggccttaccagtgaccgccttgctcctgccgctggccttgctgctccacgccgccaggccggaggtgcagctggtg | 173 |
| CD8-41BB1XX | gagtctgggggaggcctggtcaagcctggggggtccctgagactctcctgtgcagcctctggattcaccttcagtagg | |
| 3XFOXP3NFAT | tatggcatgagctgggtccgccaggctccagggaagaggctggagtgggtctcaaccattagtggcggaggcacct | |
| mCMV | acatatactacccagactcagtgaagggccgattcaccatctccagagacaacgccaagaacaccctgtatctgcaaat | |
| IL18Ralpha | gaacagcctgagagccgaggacacggctgtgtattactgtaccagagataactatggccgcaactatgattatggcatg | |
| | gattattggggccagggcaccctggtgaccgtgagcagcggtggtggcggttccggcggtggcggttccggtggtg | |
| | gcggttccgaaattgtgttgacacagtctccagccaccctgtctttgtctccaggggaaagagccaccctcacctgcag | |
| | cgccaccagcagtgttagctacatccactggtatcaacagaggcctggccagagccccaggctcctcatctatagcac | |
| | ctccaacctggccagcggcatcccagccaggttcagtggcagtgggtctgggagcgactacactctcaccatcagca | |
| | gcctagagcctgaagattttgcagtttattactgtcagcagcgtagcagctcccctttcacctttggcagcggcaccaaa | |
| | gtggaaattaaaacaacaacccctgcccccagacctcctaccccagcccctacaattgccagccagcctctgagcctg | |
| | aggcccgaggcttgtagacctgctgctggcggagccgtgcacaccagaggactggatttcgcctgcgacatctacatc | |
| | tgggcgcccttggccgggacttgtggggtccttctcctgtcactggttatcaccctttactgcaaacggggcagaaaga | |
| | aactcctgtatatattcaaacaaccatttatgagaccagtacaaactactcaagaggaagatggctgtagctgccgatttc | |
| | cagaagaagaagaaggaggatgtgaactgagagtgaagttcagcaggagcgcagacgcccccgcgtacaagcag | |
| | ggccagaaccagctctataacgagctcaatctaggacgaagagaggagtacgatgttttggacaagagacgtggccg | |
| | ggaccctgagatggggggaaagccgagaaggaagaaccctcaggaaggcctgttcaatgaactgcagaaagataa | |
| | gatggcggaggccttcagtgagattgggatgaaaggcgagcgccggaggggcaaggggcacgatggccttttcca | |
| | gggtctcagtacagccaccaaggacaccttcgacgcccttcacatgcaggccctgccccctcgctgataagtttaaact | |
| | gccagaacatttctctggcctaactggccggtaccggcttcattttttccatttactgcagaggcttcattttttccatttactg | |
| | cagaggcttcattttttccatttactgcagaactagttaggcgtgtacggtgggaggcctatataagcagagctcgtttagt | |
| | gaaccgtcagatcgcctggagacgccatccacgctgttttgacctccatagaagacaccgggaccgatccagcctcg | |
| | agagacccaatgctagccaccatgaattgtagagaattacccttgaccctttgggtgcttatatctgtaagcactgcaga | |
| | atcttgtacttcacgtccccacattactgtggttgaaggggaacctttctatctgaaacattgctcgtgttcacttgcacatg | |
| | agattgaaacaaccaccaaaagctggtacaaaagcagtggatcacaggaacatgtggagctgaacccaaggagttcc | |
| | agcagaattgctttgcatgattgtgttttggagttttggccagttgagttgaatgacacaggatcttactttttccaaatgaaa | |
| | aattatactcagaaatggaaattaaatgtcatcagaagaaataaacacagctgtttcactgaaagacaagtaactagtaaa | |
| | attgtggaagttaaaaaattttttcagataacctgtgaaaacagttactatcaaacactggtcaacagcacatcattgtataa | |
| | gaactgtaaaaagctactactggagaacaataaaaacccaacgataaagaagaacgccgagtttgaagatcaggggt | |
| | attactcctgcgtgcatttccttcatcataatggaaaactatttaatatcaccaaaaccttcaatataacaatagtggaagatc | |
| | gcagtaatatagttccggttcttcttggaccaaagcttaaccatgttgcagtggaattaggaaaaaacgtaaggctcaact | |
| | gctctgctttgctgaatgaagaggatgtaatttattggatgttcggggaagaaaatggatcggatcctaatatacatgaag | |
| | agaaagaaatgagaattatgactccagaaggcaaatggcatgcttcaaaagtattgagaattgaaaatattggtgaaag | |
| | caatctaaatgttttatataattgcactgtggccagcacgggaggcacagacaccaaaagcttcatcttggtgagaaaag | |
| | cagacatggctgatatcccaggccacgtcttcacaagaggaatgatcatagctgttttgatcttggtggcagtagtgtgc | |
| | ctagtgactgtgtgtgtcatttatagagttgacttggttctattttatagacatttaacgagaagagatgaaacattaacagat | |
| | ggaaaaacatatgatgcttttgtgtcttacctaaaagaatgccgacctgaaaatggagaggagcacacctttgctgtgga | |
| | gattttgcccagggtgttggagaaacattttgggtataagttatgcatatttgaaagggatgtagtgcctggaggagctgtt | |
| | gttgatgaaatccactcactgatagagaaaagccgaagactaatcattgtcctaagtaaaagttatatgtctaatgaggtc | |
| | aggtatgaacttgaaagtggactccatgaagcattggtggaaagaaaaattaaaataatcttaattgaatttacacctgtta | |
| | ctgacttcacattcttgccccaatcactaaagcttttgaaatctcacagagttctgaagtggaaggccgataaatctctttct | |
| | tataactcaaggttctggaagaaccttctttacttaatgcctgcaaaaacagtcaagccaggtagagacgaaccggaagt | |
| | cttgcctgttctttccgagtcttgataa | |
| CAR E6 CD8- | atggccttaccagtgaccgccttgctcctgccgctggccttgctgctccacgccgccaggccggaggtgcagctggtg | 174 |
| CD8-41BB1XX | gagtctgggggaggcctggtcaagcctggggggtccctgagactctcctgtgcagcctctggattcaccttcagtagg | |
| 6XIL12NFAT | tatggcatgagctgggtccgccaggctccagggaagaggctggagtgggtctcaaccattagtggcggaggcacct | |
| mCMV | acatatactacccagactcagtgaagggccgattcaccatctccagagacaacgccaagaacaccctgtatctgcaaat | |
| IL18Ralpha | gaacagcctgagagccgaggacacggctgtgtattactgtaccagagataactatggccgcaactatgattatggcatg | |
| | gattattggggccagggcaccctggtgaccgtgagcagcggtggtggcggttccggcggtggcggttccggtggtg | |
| | gcggttccgaaattgtgttgacacagtctccagccaccctgtctttgtctccaggggaaagagccaccctcacctgcag | |
| | cgccaccagcagtgttagctacatccactggtatcaacagaggcctggccagagccccaggctcctcatctatagcac | |
| | ctccaacctggccagcggcatcccagccaggttcagtggcagtgggtctgggagcgactacactctcaccatcagca | |
| | gcctagagcctgaagattttgcagtttattactgtcagcagcgtagcagctcccctttcacctttggcagcggcaccaaa | |
| | gtggaaattaaaacaacaacccctgcccccagacctcctaccccagcccctacaattgccagccagcctctgagcctg | |
| | aggcccgaggcttgtagacctgctgctggcggagccgtgcacaccagaggactggatttcgcctgcgacatctacatc | |
| | tgggcgcccttggccgggacttgtggggtccttctcctgtcactggttatcaccctttactgcaaacggggcagaaaga | |
| | aactcctgtatatattcaaacaaccatttatgagaccagtacaaactactcaagaggaagatggctgtagctgccgatttc | |
| | cagaagaagaagaaggaggatgtgaactgagagtgaagttcagcaggagcgcagacgcccccgcgtacaagcag | |
| | ggccagaaccagctctataacgagctcaatctaggacgaagagaggagtacgatgttttggacaagagacgtggccg | |
| | ggaccctgagatggggggaaagccgagaaggaagaaccctcaggaaggcctgttcaatgaactgcagaaagataa | |
| | gatggcggaggccttcagtgagattgggatgaaaggcgagcgccggaggggcaaggggcacgatggccttttcca | |
| | gggtctcagtacagccaccaaggacaccttcgacgcccttcacatgcaggccctgccccctcgctgataagtttaaact | |
| | gccagaacatttctctggcctaactggccggtaccggaggaaaaactgtttcatacagaaggcgtggaggaaaaactg | |
| | tttcatacagaaggcgtggaggaaaaactgtttcatacagaaggcgtggaggaaaaactgtttcatacagaaggcgtg | |
| | gaggaaaaactgtttcatacagaaggcgtggaggaaaaactgtttcatacagaaggcgtactagttaggcgtgtacggt | |
| | gggaggcctatataagcagagctcgtttagtgaaccgtcagatcgcctggagacgccatccacgctgttttgacctcca | |
| | tagaagacaccgggaccgatccagcctcgagagacccaatgctagccaccatgaattgtagagaattacccttgacc | |
| | ctttgggtgcttatatctgtaagcactgcagaatcttgtacttcacgtccccacattactgtggttgaaggggaacctttctat | |
| | ctgaaacattgctcgtgttcacttgcacatgagattgaaacaaccaccaaaagctggtacaaaagcagtggatcacagg | |
| | aacatgtggagctgaacccaaggagttccagcagaattgctttgcatgattgtgttttggagttttggccagttgagttgaa | |
| | tgacacaggatcttactttttccaaatgaaaaattatactcagaaatggaaattaaatgtcatcagaagaaataaacacag | |
| | ctgtttcactgaaagacaagtaactagtaaaattgtggaagttaaaaaattttttcagataacctgtgaaaacagttactatc | |
| | aaacactggtcaacagcacatcattgtataagaactgtaaaaagctactactggagaacaataaaaacccaacgataaa | |
| | gaagaacgccgagtttgaagatcaggggtattactcctgcgtgcatttccttcatcataatggaaaactatttaatatcacc | |
| | aaaaccttcaatataacaatagtggaagatcgcagtaatatagttccggttcttcttggaccaaagcttaaccatgttgcag | |
| | tggaattaggaaaaaacgtaaggctcaactgctctgctttgctgaatgaagaggatgtaatttattggatgttcggggaa | |
| | gaaaatggatcggatcctaatatacatgaagagaaagaaatgagaattatgactccagaaggcaaatggcatgcttcaa | |
| | aagtattgagaattgaaaatattggtgaaagcaatctaaatgttttatataattgcactgtggccagcacgggaggcacag | |
| | acaccaaaagcttcatcttggtgagaaaagcagacatggctgatatcccaggccacgtcttcacaagaggaatgatcat | |
| | agctgttttgatcttggtggcagtagtgtgcctagtgactgtgtgtgtcatttatagagttgacttggttctattttatagacatt | |
| | taacgagaagagatgaaacattaacagatggaaaaacatatgatgcttttgtgtcttacctaaaagaatgccgacctgaa | |
| | aatggagaggagcacacctttgctgtggagattttgcccagggtgttggagaaacattttgggtataagttatgcatattt | |
| | gaaagggatgtagtgcctggaggagctgttgttgatgaaatccactcactgatagagaaaagccgaagactaatcattg | |
| | tcctaagtaaaagttatatgtctaatgaggtcaggtatgaacttgaaagtggactccatgaagcattggtggaaagaaaa | |
| | attaaaataatcttaattgaatttacacctgttactgacttcacattcttgccccaatcactaaagcttttgaaatctcacagag | |
| | ttctgaagtggaaggccgataaatctctttcttataactcaaggttctggaagaaccttctttacttaatgcctgcaaaaaca | |
| | gtcaagccaggtagagacgaaccggaagtcttgcctgttctttccgagtcttgataa | |
| CAR E6 CD8- | atggccttaccagtgaccgccttgctcctgccgctggccttgctgctccacgccgccaggccggaggtgcagctggtg | 175 |
| CD8-41BB1XX | gagtctgggggaggcctggtcaagcctggggggtccctgagactctcctgtgcagcctctggattcaccttcagtagg | |
| 3XIL12NFAT | tatggcatgagctgggtccgccaggctccagggaagaggctggagtgggtctcaaccattagtggcggaggcacct | |
| mCMV | acatatactacccagactcagtgaagggccgattcaccatctccagagacaacgccaagaacaccctgtatctgcaaat | |
| IL18Ralpha | gaacagcctgagagccgaggacacggctgtgtattactgtaccagagataactatggccgcaactatgattatggcatg | |
| | gattattggggccagggcaccctggtgaccgtgagcagcggtggtggcggttccggcggtggcggttccggtggtg | |
| | gcggttccgaaattgtgttgacacagtctccagccaccctgtctttgtctccaggggaaagagccaccctcacctgcag | |
| | cgccaccagcagtgttagctacatccactggtatcaacagaggcctggccagagccccaggctctcatctatagcac | |
| | ctccaacctggccagcggcatcccagccaggttcagtggcagtgggtctgggagcgactacactctcaccatcagca | |
| | gcctagagcctgaagattttgcagtttattactgtcagcagcgtagcagctcccctttcacctttggcagcggcaccaaa | |
| | gtggaaattaaaacaacaacccctgcccccagacctcctaccccagcccctacaattgccagccagcctctgagcctg | |
| | aggcccgaggcttgtagacctgctgctggcggagccgtgcacaccagaggactggatttcgcctgcgacatctacatc | |
| | tgggcgcccttggccgggacttgtggggtccttctcctgtcactggttatcaccctttactgcaaacggggcagaaaga | |
| | aactcctgtatatattcaaacaaccatttatgagaccagtacaaactactcaagaggaagatggctgtagctgccgatttc | |
| | cagaagaagaagaaggaggatgtgaactgagagtgaagttcagcaggagcgcagacgcccccgcgtacaagcag | |
| | ggccagaaccagctctataacgagctcaatctaggacgaagagaggagtacgatgttttggacaagagacgtggccg | |
| | ggaccctgagatggggggaaagccgagaaggaagaaccctcaggaaggcctgttcaatgaactgcagaaagataa | |
| | gatggcggaggccttcagtgagattgggatgaaaggcgagcgccggaggggcaaggggcacgatggccttttcca | |
| | gggtctcagtacagccaccaaggacaccttcgacgcccttcacatgcaggccctgccccctcgctgataagtttaaact | |
| | gccagaacatttctctggcctaactggccggtaccggaggaaaaactgtttcatacagaaggcgtggaggaaaaactg | |
| | tttcatacagaaggcgtggaggaaaaactgtttcatacagaaggcgtactagttaggcgtgtacggtgggaggcctata | |
| | taagcagagctcgtttagtgaaccgtcagatcgcctggagacgccatccacgctgttttgacctccatagaagacaccg | |
| | ggaccgatccagcctcgagagacccaatgctagccaccatgaattgtagagaattacccttgaccctttgggtgcttat | |
| | atctgtaagcactgcagaatcttgtacttcacgtccccacattactgtggttgaaggggaacctttctatctgaaacattgct | |
| | cgtgttcacttgcacatgagattgaaacaaccaccaaaagctggtacaaaagcagtggatcacaggaacatgtggagc | |
| | tgaacccaaggagttccagcagaattgctttgcatgattgtgttttggagttttggccagttgagttgaatgacacaggatc | |
| | ttactttttccaaatgaaaaattatactcagaaatggaaattaaatgtcatcagaagaaataaacacagtgtttcactgaaa | |
| | gacaagtaactagtaaaattgtggaagttaaaaaattttttcagataacctgtgaaaacagttactatcaaacactggtcaa | |
| | cagcacatcattgtataagaactgtaaaaagctactactggagaacaataaaaacccaacgataaagaagaacgccga | |
| | gtttgaagatcaggggtattactcctgcgtgcatttccttcatcataatggaaaactatttaatatcaccaaaaccttcaatat | |
| | aacaatagtggaagatcgcagtaatatagttccggttcttcttggaccaaagcttaaccatgttgcagtggaattaggaaa | |
| | aaacgtaaggctcaactgctctgctttgctgaatgaagaggatgtaatttattggatgttcggggaagaaaatggatc gg | |
| | atcctaatatacatgaagagaaagaaatgagaattatgactccagaaggcaaatggcatgcttcaaaagtattgagaatt | |
| | gaaaatattggtgaaagcaatctaaatgttttatataattgcactgtggccagcacgggaggcacagacaccaaaagctt | |
| | catcttggtgagaaaagcagacatggctgatatcccaggccacgtcttcacaagaggaatgatcatagctgttttgatctt | |
| | ggtggcagtagtgtgcctagtgactgtgtgtgtcatttatagagttgacttggttctattttatagacatttaacgagaagag | |
| | atgaaacattaacagatggaaaaacatatgatgcttttgtgtcttacctaaaagaatgccgacctgaaaatggagaggag | |
| | cacacctttgctgtggagattttgcccagggtgttggagaaacattttgggtataagttatgcatatttgaaagggatgtag | |
| | tgcctggaggagctgttgttgatgaaatccactcactgatagagaaaagccgaagactaatcattgtcctaagtaaaagt | |
| | tatatgtctaatgaggtcaggtatgaacttgaaagtggactccatgaagcattggtggaaagaaaaattaaaataatctta | |
| | attgaatttacacctgttactgacttcacattcttgccccaatcactaaagcttttgaaatctcacagagttctgaagtggaa | |
| | ggccgataaatctctttcttataactcaaggttctggaagaaccttctttacttaatgcctgcaaaaacagtcaagccaggt | |
| | agagacgaaccggaagtcttgcctgttctttccgagtcttgataa | |
| CAR C2 CD8- | atggccttaccagtgaccgccttgctcctgccgctggccttgctgctccacgccgccaggccggaggtgcagctggtg | 176 |
| CD8-41BB1XX | gagtctgggggaggcctggtcaagcctggggggtccctgagactctcctgtgcagcctctggattcaccttcagtggc | |
| 6XFOXP3NFAT | tatgccatgagctgggtccgccaggctccagggaaggggctggagtgggtctcaaccattagtagtggcggaaccta | |
| mCMV | catatactaccccgactcagtgaagggccgattcaccatctccagagacaacgccaagaactcactgtatctgcaaatg | |
| IL18Ralpha-T2A- | aacagcctgagagccgaggacacggccgtgtattactgtgcgagacttgggggggataattactacgaatacttcgat | |
| IL18Rb | gtctggggcaaagggaccacggtcaccgtctcctccggcggtggcggatccggcggtggcggatccggcggtggc | |
| | ggatccgacattgtgctgacccagtctccagcctccttggccgtgtctccaggacagagggccaccatcacctgcaga | |
| | gccagtaagagtgtcagtaccagcggatactcctacatgcactggtatcagcagaaaccaggacaacctcctaaactc | |
| | ctgatttacctggcatccaatctggagagcggggtcccagccaggttcagcggcagtgggtctgggaccgatttcacc | |
| | ctcacaattaatcctgtggaagctaatgatactgcaaattattactgtcagcacagtagggagctgcctttcacattcggc | |
| | ggagggaccaaggtggagatcaaacgaactacaacaacccctgcccccagacctcctaccccagcccctacaattg | |
| | ccagccagcctctgagcctgaggcccgaggcttgtagacctgctgctggcggagccgtgcacaccagaggactgga | |
| | tttcgcctgcgacatctacatctgggcgcccttggccgggacttgtggggtccttctcctgtcactggttatcaccctttact | |
| | gcaaacggggcagaaagaaactcctgtatatattcaaacaaccatttatgagaccagtacaaactactcaagaggaag | |
| | atggctgtagctgccgatttccagaagaagaagaaggaggatgtgaactgagagtgaagttcagcaggagcgcaga | |
| | cgcccccgcgtacaagcagggccagaaccagctctataacgagctcaatctaggacgaagagaggagtacgatgttt | |
| | tggacaagagacgtggccgggaccctgagatggggggaaagccgagaaggaagaaccctcaggaaggcctgttc | |
| | aatgaactgcagaaagataagatggcggaggccttcagtgagattgggatgaaaggcgagcgccggaggggcaag | |
| | gggcacgatggccttttccagggtctcagtacagccaccaaggacaccttcgacgcccttcacatgcaggccctgccc | |
| | cctcgctgataagtttaaactgccagaacatttctctggcctaactggccggtaccggcttcattttttccatttactgcaga | |
| | ggcttcattttttccatttactgcagaggcttcattttttccatttactgcagaggcttcattttttccatttactgcagaggcttc | |
| | attttttccatttactgcagaggcttcattttttccatttactgcagaactagttaggcgtgtacggtgggaggcctatataag | |
| | cagagctcgtttagtgaaccgtcagatcgcctggagacgccatccacgctgttttgacctccatagaagacaccggga | |
| | ccgatccagcctcgagagacccaatgctagccaccatgaattgtagagaattacccttgaccctttgggtgcttatatct | |
| | gtaagcactgcagaatcttgtacttcacgtccccacattactgtggttgaaggggaacctttctatctgaaacattgctcgt | |
| | gttcacttgcacatgagattgaaacaaccaccaaaagctggtacaaaagcagtggatcacaggaacatgtggagctga | |
| | acccaaggagttccagcagaattgctttgcatgattgtgttttggagttttggccagttgagttgaatgacacaggatctta | |
| | ctttttccaaatgaaaaattatactcagaaatggaaattaaatgtcatcagaagaaataaacacagctgtttcactgaaaga | |
| | caagtaactagtaaaattgtggaagttaaaaaattttttcagataacctgtgaaaacagttactatcaaacactggtcaaca | |
| | gcacatcattgtataagaactgtaaaaagctactactggagaacaataaaaacccaacgataaagaagaacgccgagt | |
| | ttgaagatcaggggtattactcctgcgtgcatttccttcatcataatggaaaactatttaatatcaccaaaaccttcaatataa | |
| | caatagtggaagatcgcagtaatatagttccggttcttcttggaccaaagcttaaccatgttgcagtggaattaggaaaaa | |
| | acgtaaggctcaactgctctgctttgctgaatgaagaggatgtaatttattggatgttcggggaagaaaatggatcggat | |
| | cctaatatacatgaagagaaagaaatgagaattatgactccagaaggcaaatggcatgcttcaaaagtattgagaattga | |
| | aaatattggtgaaagcaatctaaatgttttatataattgcactgtggccagcacgggaggcacagacaccaaaagcttca | |
| | tcttggtgagaaaagcagacatggctgatatcccaggccacgtcttcacaagaggaatgatcatagctgttttgatcttgg | |
| | tggcagtagtgtgcctagtgactgtgtgtgtcatttatagagttgacttggttctattttatagacatttaacgagaagagatg | |
| | aaacattaacagatggaaaaacatatgatgcttttgtgtcttacctaaaagaatgccgacctgaaaatggagaggagcac | |
| | acctttgctgtggagattttgcccagggtgttggagaaacattttgggtataagttatgcatatttgaaagggatgtagtgc | |
| | ctggaggagctgttgttgatgaaatccactcactgatagagaaaagccgaagactaatcattgtcctaagtaaaagttata | |
| | tgtctaatgaggtcaggtatgaacttgaaagtggactccatgaagcattggtggaaagaaaaattaaaataatcttaattg | |
| | aatttacacctgttactgacttcacattcttgccccaatcactaaagcttttgaaatctcacagagttctgaagtggaaggcc | |
| | gataaatctctttcttataactcaaggttctggaagaaccttctttacttaatgcctgcaaaaacagtcaagccaggtagag | |
| | acgaaccggaagtcttgcctgttctttccgagtctggaagcggtgagggaagaggtagcctgctcacgtgcggtgatg | |
| | tggaggagaaccctgggcctatgctctgtttgggctggatatttctttggcttgttgcaggagagcgaattaaaggattta | |
| | atatttcaggttgttccacaaaaaaactcctttggacatattctacaaggagtgaagaggaatttgtcttattttgtgatttacc | |
| | agagccacagaaatcacatttctgccacagaaatcgactctcaccaaaacaagtccctgagcacctgcccttcatgggt | |
| | agtaacgacctatctgatgtccaatggtaccaacaaccttcgaatggagatccattagaggacattaggaaaagctatcc | |
| | tcacatcattcaggacaaatgtacccttcactttttgaccccaggggtgaataattctgggtcatatatttgtagacccaag | |
| | atgattaagagcccctatgatgtagcctgttgtgtcaagatgattttagaagttaagccccagacaaatgcatcctgtgagt | |
| | attccgcatcacataagcaagacctacttcttgggagcactggctctatttcttgccccagtctcagctgccaaagtgatg | |
| | cacaaagtccagcggtaacctggtacaagaatggaaaactcctctctgtggaaaggagcaaccgaatcgtagtggatg | |
| | aagtttatgactatcaccagggcacatatgtatgtgattacactcagtcggatactgtgagttcgtggacagtcagagctg | |
| | ttgttcaagtgagaaccattgtgggagacactaaactcaaaccagatattctggatcctgtcgaggacacactggaagta | |
| | gaacttggaaagcctttaactattagctgcaaagcacgatttggctttgaaagggtctttaaccctgtcataaaatggtaca | |
| | tcaaagattctgacctagagtgggaagtctcagtacctgaggcgaaaagtattaaatccactttaaaggatgaaatcattg | |
| | agcgtaatatcatcttggaaaaagtcactcagcgtgatcttcgcaggaagtttgtttgctttgtccagaactccattggaaa | |
| | cacaacccagtccgtccaactgaaagaaaagagaggagtggtgctcctgtacatcctgcttggcaccatcgggaccct | |
| | ggtggccgtgctggcggcgagtgccctcctctacaggcactggattgaaatagtgctgctgtaccggacctaccagag | |
| | caaggatcagacgcttggggataaaaaggattttgatgctttcgtatcctatgcaaaatggagctcttttccaagtgaggc | |
| | cacttcatctctgagtgaagaacacttggccctgagcctatttcctgatgttttagaaaacaaatatggatatagcctgtgtt | |
| | tgcttgaaagagatgtggctccaggaggagtgtatgcagaagacattgtgagcattattaagagaagcagaagaggaa | |
| | tatttatcttgagccccaactatgtcaatggacccagtatctttgaactacaagcagcagtgaatcttgccttggatgatca | |
| | aacactgaaactcattttaattaagttctgttacttccaagagccagagtctctacctcatctcgtgaaaaaagctctcagg | |
| | gttttgcccacagttacttggagaggcttaaaatcagttcctcccaattctaggttctgggccaaaatgcgctaccacatg | |
| | cctgtgaaaaactctcagggattcacgtggaaccagctcagaattacctctaggatttttcagtggaaaggactcagtag | |
| | aacagaaaccactgggaggagctcccagcctaaggaatggtgataa | |
| CAR C2 CD8- | atggccttaccagtgaccgccttgctcctgccgctggccttgctgctccacgccgccaggccggaggtgcagctggtg | 177 |
| CD8-41BB1XX | gagtctgggggaggcctggtcaagcctggggggtccctgagactctcctgtgcagcctctggattcaccttcagtggc | |
| 3XFOXP3NFAT | tatgccatgagctgggtccgccaggctccagggaaggggctggagtgggtctcaaccattagtagtggcggaaccta | |
| mCMV | catatactaccccgactcagtgaagggccgattcaccatctccagagacaacgccaagaactcactgtatctgcaaatg | |
| IL18Ralpha-T2A- | aacagcctgagagccgaggacacggccgtgtattactgtgcgagacttgggggggataattactacgaatacttcgat | |
| IL18Rb | gtctggggcaaagggaccacggtcaccgtctcctccggcggtggcggatccggcggtggcggatccggcggtggc | |
| | ggatccgacattgtgctgacccagtctccagcctccttggccgtgtctccaggacagagggccaccatcacctgcaga | |
| | gccagtaagagtgtcagtaccagcggatactcctacatgcactggtatcagcagaaaccaggacaacctcctaaactc | |
| | ctgatttacctggcatccaatctggagagcggggtcccagccaggttcagcggcagtgggtctgggaccgatttcacc | |
| | ctcacaattaatcctgtggaagctaatgatactgcaaattattactgtcagcacagtagggagctgcctttcacattcggc | |
| | ggagggaccaaggtggagatcaaacgaactacaacaacccctgcccccagacctcctaccccagcccctacaattg | |
| | ccagccagcctctgagcctgaggcccgaggcttgtagacctgctgctggcggagccgtgcacaccagaggactgga | |
| | tttcgcctgcgacatctacatctgggcgcccttggccgggacttgtggggtccttctcctgtcactggttatcaccctttact | |
| | gcaaacggggcagaaagaaactcctgtatatattcaaacaaccatttatgagaccagtacaaactactcaagaggaag | |
| | atggctgtagctgccgatttccagaagaagaagaaggaggatgtgaactgagagtgaagttcagcaggagcgcaga | |
| | cgcccccgcgtacaagcagggccagaaccagctctataacgagctcaatctaggacgaagagaggagtacgatgttt | |
| | tggacaagagacgtggccgggaccctgagatggggggaaagccgagaaggaagaaccctcaggaaggcctgttc | |
| | aatgaactgcagaaagataagatggcggaggccttcagtgagattgggatgaaaggcgagcgccggaggggcaag | |
| | gggcacgatggccttttccagggtctcagtacagccaccaaggacaccttcgacgcccttcacatgcaggccctgccc | |
| | cctcgctgataagtttaaactgccagaacatttctctggcctaactggccggtaccggcttcattttttccatttactgcaga | |
| | ggcttcattttttccatttactgcagaggcttcattttttccatttactgcagaactagttaggcgtgtacggtgggaggccta | |
| | tataagcagagctcgtttagtgaaccgtcagatcgcctggagacgccatccacgctgttttgacctccatagaagacac | |
| | cgggaccgatccagcctcgagagacccaatgctagccaccatgaattgtagagaattacccttgaccctttgggtgctt | |
| | atatctgtaagcactgcagaatcttgtacttcacgtccccacattactgtggttgaaggggaacctttctatctgaaacattg | |
| | ctcgtgttcacttgcacatgagattgaaacaaccaccaaaagctggtacaaaagcagtggatcacaggaacatgtgga | |
| | gctgaacccaaggagttccagcagaattgctttgcatgattgtgttttggagttttggccagttgagttgaatgacacagg | |
| | atcttactttttccaaatgaaaaattatactcagaaatggaaattaaatgtcatcagaagaaataaacacagctgtttcactg | |
| | aaagacaagtaactagtaaaattgtggaagttaaaaaattttttcagataacctgtgaaaacagttactatcaaacactggt | |
| | caacagcacatcattgtataagaactgtaaaaagctactactggagaacaataaaaacccaacgataaagaagaacgc | |
| | cgagtttgaagatcaggggtattactcctgcgtgcatttccttcatcataatggaaaactatttaatatcaccaaaaccttca | |
| | atataacaatagtggaagatcgcagtaatatagttccggttcttcttggaccaaagcttaaccatgttgcagtggaattagg | |
| | aaaaaacgtaaggctcaactgctctgctttgctgaatgaagaggatgtaatttattggatgttcggggaagaaaatggat | |
| | cggatcctaatatacatgaagagaaagaaatgagaattatgactccagaaggcaaatggcatgcttcaaaagtattgag | |
| | aattgaaaatattggtgaaagcaatctaaatgttttatataattgcactgtggccagcacgggaggcacagacaccaaaa | |
| | gcttcatcttggtgagaaaagcagacatggctgatatcccaggccacgtcttcacaagaggaatgatcatagctgttttg | |
| | atcttggtggcagtagtgtgcctagtgactgtgtgtgtcatttatagagttgacttggttctattttatagacatttaacgaga | |
| | agagatgaaacattaacagatggaaaaacatatgatgcttttgtgtcttacctaaaagaatgccgacctgaaaatggaga | |
| | ggagcacacctttgctgtggagattttgcccagggtgttggagaaacattttgggtataagttatgcatatttgaaaggga | |
| | tgtagtgcctggaggagctgttgttgatgaaatccactcactgatagagaaaagccgaagactaatcattgtcctaagta | |
| | aaagttatatgtctaatgaggtcaggtatgaacttgaaagtggactccatgaagcattggtggaaagaaaaattaaaata | |
| | atcttaattgaatttacacctgttactgacttcacattcttgccccaatcactaaagcttttgaaatctcacagagttctgaagt | |
| | ggaaggccgataaatctctttcttataactcaaggttctggaagaaccttctttacttaatgcctgcaaaaacagtcaagcc | |
| | aggtagagacgaaccggaagtcttgcctgttctttccgagtctggaagcggtgagggaagaggtagcctgctcacgtg | |
| | cggtgatgtggaggagaaccctgggcctatgctctgtttgggctggatatttctttggcttgttgcaggagagcgaattaa | |
| | aggatttaatatttcaggttgttccacaaaaaaactcctttggacatattctacaaggagtgaagaggaatttgtcttattttgt | |
| | gatttaccagagccacagaaatcacatttctgccacagaaatcgactctcaccaaaacaagtccctgagcacctgccctt | |
| | catgggtagtaacgacctatctgatgtccaatggtaccaacaaccttcgaatggagatccattagaggacattaggaaa | |
| | agctatcctcacatcattcaggacaaatgtacccttcactttttgaccccaggggtgaataattctgggtcatatatttgtag | |
| | acccaagatgattaagagcccctatgatgtagcctgttgtgtcaagatgattttagaagttaagccccagacaaatgcatc | |
| | ctgtgagtattccgcatcacataagcaagacctacttcttgggagcactggctctatttcttgccccagtctcagctgccaa | |
| | agtgatgcacaaagtccagcggtaacctggtacaagaatggaaaactcctctctgtggaaaggagcaaccgaatcgta | |
| | gtggatgaagtttatgactatcaccagggcacatatgtatgtgattacactcagtcggatactgtgagttcgtggacagtc | |
| | agagctgttgttcaagtgagaaccattgtgggagacactaaactcaaaccagatattctggatcctgtcgaggacacact | |
| | ggaagtagaacttggaaagcctttaactattagctgcaaagcacgatttggctttgaaagggtctttaaccctgtcataaa | |
| | atggtacatcaaagattctgacctagagtgggaagtctcagtacctgaggcgaaaagtattaaatccactttaaaggatg | |
| | aaatcattgagcgtaatatcatcttggaaaaagtcactcagcgtgatcttcgcaggaagtttgtttgctttgtccagaactcc | |
| | attggaaacacaacccagtccgtccaactgaaagaaaagagaggagtggtgctcctgtacatcctgcttggcaccatc | |
| | gggaccctggtggccgtgctggcggcgagtgccctcctctacaggcactggattgaaatagtgctgctgtaccggacc | |
| | taccagagcaaggatcagacgcttggggataaaaaggattttgatgctttcgtatcctatgcaaaatggagctcttttcca | |
| | agtgaggccacttcatctctgagtgaagaacacttggccctgagcctatttcctgatgttttagaaaacaaatatggatata | |
| | gcctgtgtttgcttgaaagagatgtggctccaggaggagtgtatgcagaagacattgtgagcattattaagagaagcag | |
| | aagaggaatatttatcttgagccccaactatgtcaatggacccagtatctttgaactacaagcagcagtgaatcttgccttg | |
| | gatgatcaaacactgaaactcattttaattaagttctgttacttccaagagccagagtctctacctcatctcgtgaaaaaag | |
| | ctctcagggttttgcccacagttacttggagaggcttaaaatcagttcctcccaattctaggttctgggccaaaatgcgcta | |
| | ccacatgcctgtgaaaaactctcagggattcacgtggaaccagctcagaattacctctaggatttttcagtggaaaggac | |
| | tcagtagaacagaaaccactgggaggagctcccagcctaaggaatggtgataa | |
| CAR C2 CD8- | atggccttaccagtgaccgccttgctcctgccgctggccttgctgctccacgccgccaggccggaggtgcagctggtg | 178 |
| CD8-41BB1XX | gagtctgggggaggcctggtcaagcctggggggtccctgagactctcctgtgcagcctctggattcaccttcagtggc | |
| 6XIL12NFAT | tatgccatgagctgggtccgccaggctccagggaaggggctggagtgggtctcaaccattagtagtggcggaaccta | |
| mCMV | catatactaccccgactcagtgaagggccgattcaccatctccagagacaacgccaagaactcactgtatctgcaaatg | |
| IL18Ralpha-T2A- | aacagcctgagagccgaggacacggccgtgtattactgtgcgagacttgggggggataattactacgaatacttcgat | |
| IL18Rb | gtctggggcaaagggaccacggtcaccgtctcctccggcggtggcggatccggcggtggcggatccggcggtggc | |
| | ggatccgacattgtgctgacccagtctccagcctccttggccgtgtctccaggacagagggccaccatcacctgcaga | |
| | gccagtaagagtgtcagtaccagcggatactcctacatgcactggtatcagcagaaaccaggacaacctcctaaactc | |
| | ctgatttacctggcatccaatctggagagcggggtcccagccaggttcagcggcagtgggtctgggaccgatttcacc | |
| | ctcacaattaatcctgtggaagctaatgatactgcaaattattactgtcagcacagtagggagctgcctttcacattcggc | |
| | ggagggaccaaggtggagatcaaacgaactacaacaacccctgcccccagacctcctaccccagcccctacaattg | |
| | ccagccagcctctgagcctgaggcccgaggcttgtagacctgctgctggcggagccgtgcacaccagaggactgga | |
| | tttcgcctgcgacatctacatctgggcgcccttggccgggacttgtggggtccttctcctgtcactggttatcaccctttact | |
| | gcaaacggggcagaaagaaactcctgtatatattcaaacaaccatttatgagaccagtacaaactactcaagaggaag | |
| | atggctgtagctgccgatttccagaagaagaagaaggaggatgtgaactgagagtgaagttcagcaggagcgcaga | |
| | cgcccccgcgtacaagcagggccagaaccagctctataacgagctcaatctaggacgaagagaggagtacgatgttt | |
| | tggacaagagacgtggccgggaccctgagatggggggaaagccgagaaggaagaaccctcaggaaggcctgttc | |
| | aatgaactgcagaaagataagatggcggaggccttcagtgagattgggatgaaaggcgagcgccggaggggcaag | |
| | gggcacgatggccttttccagggtctcagtacagccaccaaggacaccttcgacgcccttcacatgcaggccctgccc | |
| | cctcgctgataagtttaaactgccagaacatttctctggcctaactggccggtaccggaggaaaaactgtttcatacaga | |
| | aggcgtggaggaaaaactgtttcatacagaaggcgtggaggaaaaactgtttcatacagaaggcgtggaggaaaaac | |
| | tgtttcatacagaaggcgtggaggaaaaactgtttcatacagaaggcgtggaggaaaaactgtttcatacagaaggcgt | |
| | actagttaggcgtgtacggtgggaggcctatataagcagagctcgtttagtgaaccgtcagatcgcctggagacgccat | |
| | ccacgctgttttgacctccatagaagacaccgggaccgatccagcctcgagagacccaatgctagccaccatgaatt | |
| | gtagagaattacccttgaccctttgggtgcttatatctgtaagcactgcagaatcttgtacttcacgtccccacattactgtg | |
| | gttgaaggggaacctttctatctgaaacattgctcgtgttcacttgcacatgagattgaaacaaccaccaaaagctggtac | |
| | aaaagcagtggatcacaggaacatgtggagctgaacccaaggagttccagcagaattgctttgcatgattgtgttttgga | |
| | gttttggccagttgagttgaatgacacaggatcttactttttccaaatgaaaaattatactcagaaatggaaattaaatgtcat | |
| | cagaagaaataaacacagctgtttcactgaaagacaagtaactagtaaaattgtggaagttaaaaaattttttcagataac | |
| | ctgtgaaaacagttactatcaaacactggtcaacagcacatcattgtataagaactgtaaaaagctactactggagaaca | |
| | ataaaaacccaacgataaagaagaacgccgagtttgaagatcaggggtattactcctgcgtgcatttccttcatcataat | |
| | ggaaaactatttaatatcaccaaaaccttcaatataacaatagtggaagatcgcagtaatatagttccggttcttcttggac | |
| | caaagcttaaccatgttgcagtggaattaggaaaaaacgtaaggctcaactgctctgctttgctgaatgaagaggatgta | |
| | atttattggatgttcggggaagaaaatggatcggatcctaatatacatgaagagaaagaaatgagaattatgactccaga | |
| | aggcaaatggcatgcttcaaaagtattgagaattgaaaatattggtgaaagcaatctaaatgttttatataattgcactgtg | |
| | gccagcacgggaggcacagacaccaaaagcttcatcttggtgagaaaagcagacatggctgatatcccaggccacg | |
| | tcttcacaagaggaatgatcatagctgttttgatcttggtggcagtagtgtgcctagtgactgtgtgtgtcatttatagagtt | |
| | gacttggttctattttatagacatttaacgagaagagatgaaacattaacagatggaaaaacatatgatgcttttgtgtctta | |
| | cctaaaagaatgccgacctgaaaatggagaggagcacacctttgctgtggagattttgcccagggtgttggagaaaca | |
| | ttttgggtataagttatgcatatttgaaagggatgtagtgcctggaggagctgttgttgatgaaatccactcactgatagag | |
| | aaaagccgaagactaatcattgtcctaagtaaaagttatatgtctaatgaggtcaggtatgaacttgaaagtggactccat | |
| | gaagcattggtggaaagaaaaattaaaataatcttaattgaatttacacctgttactgacttcacattcttgccccaatcact | |
| | aaagcttttgaaatctcacagagttctgaagtggaaggccgataaatctctttcttataactcaaggttctggaagaacctt | |
| | ctttacttaatgcctgcaaaaacagtcaagccaggtagagacgaaccggaagtcttgcctgttctttccgagtctggaag | |
| | cggtgagggaagaggtagcctgctcacgtgcggtgatgtggaggagaaccctgggcctatgctctgtttgggctggat | |
| | atttctttggcttgttgcaggagagcgaattaaaggatttaatatttcaggttgttccacaaaaaaactcctttggacatattct | |
| | acaaggagtgaagaggaatttgtcttattttgtgatttaccagagccacagaaatcacatttctgccacagaaatcgactct | |
| | caccaaaacaagtccctgagcacctgcccttcatgggtagtaacgacctatctgatgtccaatggtaccaacaaccttcg | |
| | aatggagatccattagaggacattaggaaaagctatcctcacatcattcaggacaaatgtacccttcactttttgacccca | |
| | ggggtgaataattctgggtcatatatttgtagacccaagatgattaagagcccctatgatgtagcctgttgtgtcaagatg | |
| | attttagaagttaagccccagacaaatgcatcctgtgagtattccgcatcacataagcaagacctacttcttgggagcact | |
| | ggctctatttcttgccccagtctcagctgccaaagtgatgcacaaagtccagcggtaacctggtacaagaatggaaaac | |
| | tcctctctgtggaaaggagcaaccgaatcgtagtggatgaagtttatgactatcaccagggcacatatgtatgtgattaca | |
| | ctcagtcggatactgtgagttcgtggacagtcagagctgttgttcaagtgagaaccattgtgggagacactaaactcaaa | |
| | ccagatattctggatcctgtcgaggacacactggaagtagaacttggaaagcctttaactattagctgcaaagcacgattt | |
| | ggctttgaaagggtctttaaccctgtcataaaatggtacatcaaagattctgacctagagtgggaagtctcagtacctgag | |
| | gcgaaaagtattaaatccactttaaaggatgaaatcattgagcgtaatatcatcttggaaaaagtcactcagcgtgatcttc | |
| | gcaggaagtttgtttgctttgtccagaactccattggaaacacaacccagtccgtccaactgaaagaaaagagaggagt | |
| | ggtgctcctgtacatcctgcttggcaccatcgggaccctggtggccgtgctggcggcgagtgccctcctctacaggca | |
| | ctggattgaaatagtgctgctgtaccggacctaccagagcaaggatcagacgcttggggataaaaaggattttgatgctt | |
| | tcgtatcctatgcaaaatggagctcttttccaagtgaggccacttcatctctgagtgaagaacacttggccctgagcctatt | |
| | tcctgatgttttagaaaacaaatatggatatagcctgtgtttgcttgaaagagatgtggctccaggaggagtgtatgcaga | |
| | agacattgtgagcattattaagagaagcagaagaggaatatttatcttgagccccaactatgtcaatggacccagtatctt | |
| | tgaactacaagcagcagtgaatcttgccttggatgatcaaacactgaaactcattttaattaagttctgttacttccaagagc | |
| | cagagtctctacctcatctcgtgaaaaaagctctcagggttttgcccacagttacttggagaggcttaaaatcagttcctcc | |
| | caattctaggttctgggccaaaatgcgctaccacatgcctgtgaaaaactctcagggattcacgtggaaccagctcaga | |
| | attacctctaggatttttcagtggaaaggactcagtagaacagaaaccactgggaggagctcccagcctaaggaatggt | |
| | gataa | |
| CAR C2 CD8- | atggccttaccagtgaccgccttgctcctgccgctggccttgctgctccacgccgccaggccggaggtgcagctggtg | 179 |
| CD8-41BB1XX | gagtctgggggaggcctggtcaagcctggggggtccctgagactctcctgtgcagcctctggattcaccttcagtggc | |
| 3XIL12NFAT | tatgccatgagctgggtccgccaggctccagggaaggggctggagtgggtctcaaccattagtagtggcggaaccta | |
| mCMV | catatactaccccgactcagtgaagggccgattcaccatctccagagacaacgccaagaactcactgtatctgcaaatg | |
| IL18Ralpha-T2A- | aacagcctgagagccgaggacacggccgtgtattactgtgcgagacttgggggggataattactacgaatacttcgat | |
| IL18Rb | gtctggggcaaagggaccacggtcaccgtctcctccggcggtggcggatccggcggtggcggatccggcggtggc | |
| | gccagtaagagtgtcagtaccagcggatactcctacatgcactggtatcagcagaaaccaggacaacctcctaaactc | |
| | ggatccgacattgtgctgacccagtctccagcctccttggccgtgtctccaggacagagggccaccatcacctgcaga | |
| | ctgatttacctggcatccaatctggagagcggggtcccagccaggttcagcggcagtgggtctgggaccgatttcacc | |
| | ctcacaattaatcctgtggaagctaatgatactgcaaattattactgtcagcacagtagggagctgcctttcacattcggc | |
| | ggagggaccaaggtggagatcaaacgaactacaacaacccctgcccccagacctcctaccccagcccctacaattg | |
| | ccagccagcctctgagcctgaggcccgaggcttgtagacctgctgctggcggagccgtgcacaccagaggactgga | |
| | tttcgcctgcgacatctacatctgggcgcccttggccgggacttgtggggtccttctcctgtcactggttatcaccctttact | |
| | gcaaacggggcagaaagaaactcctgtatatattcaaacaaccatttatgagaccagtacaaactactcaagaggaag | |
| | atggctgtagctgccgatttccagaagaagaagaaggaggatgtgaactgagagtgaagttcagcaggagcgcaga | |
| | cgcccccgcgtacaagcagggccagaaccagctctataacgagctcaatctaggacgaagagaggagtacgatgttt | |
| | tggacaagagacgtggccgggaccctgagatggggggaaagccgagaaggaagaaccctcaggaaggcctgttc | |
| | aatgaactgcagaaagataagatggcggaggccttcagtgagattgggatgaaaggcgagcgccggaggggcaag | |
| | gggcacgatggccttttccagggtctcagtacagccaccaaggacaccttcgacgcccttcacatgcaggccctgccc | |
| | cctcgctgataagtttaaactgccagaacatttctctggcctaactggccggtaccggaggaaaaactgtttcatacaga | |
| | aggcgtggaggaaaaactgtttcatacagaaggcgtggaggaaaaactgtttcatacagaaggcgtactagttaggcg | |
| | tgtacggtgggaggcctatataagcagagctcgtttagtgaaccgtcagatcgcctggagacgccatccacgctgtttt | |
| | gacctccatagaagacaccgggaccgatccagcctcgagagacccaatgctagccaccatgaattgtagagaattac | |
| | ccttgaccctttgggtgcttatatctgtaagcactgcagaatcttgtacttcacgtccccacattactgtggttgaagggga | |
| | acctttctatctgaaacattgctcgtgttcacttgcacatgagattgaaacaaccaccaaaagctggtacaaaagcagtgg | |
| | atcacaggaacatgtggagctgaacccaaggagttccagcagaattgctttgcatgattgtgttttggagttttggccagt | |
| | tgagttgaatgacacaggatcttactttttccaaatgaaaaattatactcagaaatggaaattaaatgtcatcagaagaaat | |
| | aaacacagctgtttcactgaaagacaagtaactagtaaaattgtggaagttaaaaaattttttcagataacctgtgaaaaca | |
| | gttactatcaaacactggtcaacagcacatcattgtataagaactgtaaaaagctactactggagaacaataaaaaccca | |
| | acgataaagaagaacgccgagtttgaagatcaggggtattactcctgcgtgcatttccttcatcataatggaaaactattt | |
| | aatatcaccaaaaccttcaatataacaatagtggaagatcgcagtaatatagttccggttcttcttggaccaaagcttaacc | |
| | atgttgcagtggaattaggaaaaaacgtaaggctcaactgctctgctttgctgaatgaagaggatgtaatttattggatgtt | |
| | cggggaagaaaatggatcggatcctaatatacatgaagagaaagaaatgagaattatgactccagaaggcaaatggc | |
| | atgcttcaaaagtattgagaattgaaaatattggtgaaagcaatctaaatgttttatataattgcactgtggccagcacggg | |
| | aggcacagacaccaaaagcttcatcttggtgagaaaagcagacatggctgatatcccaggccacgtcttcacaagag | |
| | gaatgatcatagctgttttgatcttggtggcagtagtgtgcctagtgactgtgtgtgtcatttatagagttgacttggttctatt | |
| | ttatagacatttaacgagaagagatgaaacattaacagatggaaaaacatatgatgcttttgtgtcttacctaaaagaatgc | |
| | cgacctgaaaatggagaggagcacacctttgctgtggagattttgcccagggtgttggagaaacattttgggtataagtt | |
| | atgcatatttgaaagggatgtagtgcctggaggagctgttgttgatgaaatccactcactgatagagaaaagccgaaga | |
| | ctaatcattgtcctaagtaaaagttatatgtctaatgaggtcaggtatgaacttgaaagtggactccatgaagcattggtgg | |
| | aaagaaaaattaaaataatcttaattgaatttacacctgttactgacttcacattcttgccccaatcactaaagcttttgaaatc | |
| | tcacagagttctgaagtggaaggccgataaatctctttcttataactcaaggttctggaagaaccttctttacttaatgcctg | |
| | caaaaacagtcaagccaggtagagacgaaccggaagtcttgcctgttctttccgagtctggaagcggtgagggaaga | |
| | ggtagcctgctcacgtgcggtgatgtggaggagaaccctgggcctatgctctgtttgggctggatatttctttggcttgtt | |
| | gcaggagagcgaattaaaggatttaatatttcaggttgttccacaaaaaaactcctttggacatattctacaaggagtgaa | |
| | gaggaatttgtcttattttgtgatttaccagagccacagaaatcacatttctgccacagaaatcgactctcaccaaaacaag | |
| | tccctgagcacctgcccttcatgggtagtaacgacctatctgatgtccaatggtaccaacaaccttcgaatggagatcca | |
| | ttagaggacattaggaaaagctatcctcacatcattcaggacaaatgtacccttcactttttgaccccaggggtgaataatt | |
| | ctgggtcatatatttgtagacccaagatgattaagagcccctatgatgtagcctgttgtgtcaagatgattttagaagttaag | |
| | ccccagacaaatgcatcctgtgagtattccgcatcacataagcaagacctacttcttgggagcactggctctatttcttgc | |
| | cccagtctcagctgccaaagtgatgcacaaagtccagcggtaacctggtacaagaatggaaaactcctctctgtggaa | |
| | aggagcaaccgaatcgtagtggatgaagtttatgactatcaccagggcacatatgtatgtgattacactcagtcggatac | |
| | tgtgagttcgtggacagtcagagctgttgttcaagtgagaaccattgtgggagacactaaactcaaaccagatattctgg | |
| | atcctgtcgaggacacactggaagtagaacttggaaagcctttaactattagctgcaaagcacgatttggctttgaaagg | |
| | gtctttaaccctgtcataaaatggtacatcaaagattctgacctagagtgggaagtctcagtacctgaggcgaaaagtatt | |
| | aaatccactttaaaggatgaaatcattgagcgtaatatcatcttggaaaaagtcactcagcgtgatcttcgcaggaagttt | |
| | gtttgctttgtccagaactccattggaaacacaacccagtccgtccaactgaaagaaaagagaggagtggtgctcctgt | |
| | acatcctgcttggcaccatcgggaccctggtggccgtgctggcggcgagtgccctcctctacaggcactggattgaaa | |
| | tagtgctgctgtaccggacctaccagagcaaggatcagacgcttggggataaaaaggattttgatgctttcgtatcctat | |
| | gcaaaatggagctcttttccaagtgaggccacttcatctctgagtgaagaacacttggccctgagcctatttcctgatgttt | |
| | tagaaaacaaatatggatatagcctgtgtttgcttgaaagagatgtggctccaggaggagtgtatgcagaagacattgtg | |
| | agcattattaagagaagcagaagaggaatatttatcttgagccccaactatgtcaatggacccagtatctttgaactacaa | |
| | gcagcagtgaatcttgccttggatgatcaaacactgaaactcattttaattaagttctgttacttccaagagccagagtctct | |
| | acctcatctcgtgaaaaaagctctcagggttttgcccacagttacttggagaggcttaaaatcagttcctcccaattctagg | |
| | ttctgggccaaaatgcgctaccacatgcctgtgaaaaactctcagggattcacgtggaaccagctcagaattacctctag | |
| | gatttttcagtggaaaggactcagtagaacagaaaccactgggaggagctcccagcctaaggaatggtgataa | |
| CAR E6 CD8- | atggccttaccagtgaccgccttgctcctgccgctggccttgctgctccacgccgccaggccggaggtgcagctggtg | 180 |
| CD8-41BB1XX | gagtctgggggaggcctggtcaagcctggggggtccctgagactctcctgtgcagcctctggattcaccttcagtagg | |
| 6XFOXP3NFAT | tatggcatgagctgggtccgccaggctccagggaagaggctggagtgggtctcaaccattagtggcggaggcacct | |
| mCMV | acatatactacccagactcagtgaagggccgattcaccatctccagagacaacgccaagaacaccctgtatctgcaaat | |
| IL18Ralpha-T2A- | gattattggggccagggcaccctggtgaccgtgagcagcggtggtggcggttccggcggtggcggttccggtggtg | |
| IL18Rb | gaacagcctgagagccgaggacacggctgtgtattactgtaccagagataactatggccgcaactatgattatggcatg | |
| | gcggttccgaaattgtgttgacacagtctccagccaccctgtctttgtctccaggggaaagagccaccctcacctgcag | |
| | cgccaccagcagtgttagctacatccactggtatcaacagaggcctggccagagccccaggctcctcatctatagcac | |
| | ctccaacctggccagcggcatcccagccaggttcagtggcagtgggtctgggagcgactacactctcaccatcagca | |
| | gcctagagcctgaagattttgcagtttattactgtcagcagcgtagcagctcccctttcacctttggcagcggcaccaaa | |
| | gtggaaattaaaacaacaacccctgcccccagacctcctaccccagcccctacaattgccagccagcctctgagcctg | |
| | aggcccgaggcttgtagacctgctgctggcggagccgtgcacaccagaggactggatttcgcctgcgacatctacatc | |
| | tgggcgcccttggccgggacttgtggggtccttctcctgtcactggttatcaccctttactgcaaacggggcagaaaga | |
| | aactcctgtatatattcaaacaaccatttatgagaccagtacaaactactcaagaggaagatggctgtagctgccgatttc | |
| | cagaagaagaagaaggaggatgtgaactgagagtgaagttcagcaggagcgcagacgcccccgcgtacaagcag | |
| | ggccagaaccagctctataacgagctcaatctaggacgaagagaggagtacgatgttttggacaagagacgtggccg | |
| | ggaccctgagatggggggaaagccgagaaggaagaaccctcaggaaggcctgttcaatgaactgcagaaagataa | |
| | gatggcggaggccttcagtgagattgggatgaaaggcgagcgccggaggggcaaggggcacgatggccttttcca | |
| | gggtctcagtacagccaccaaggacaccttcgacgcccttcacatgcaggccctgccccctcgctgataagtttaaact | |
| | gccagaacatttctctggcctaactggccggtaccggcttcattttttccatttactgcagaggcttcattttttccatttactg | |
| | cagaggcttcattttttccatttactgcagaggcttcattttttccatttactgcagaggcttcattttttccatttactgcagag | |
| | gcttcattttttccatttactgcagaactagttaggcgtgtacggtgggaggcctatataagcagagctcgtttagtgaacc | |
| | gtcagatcgcctggagacgccatccacgctgttttgacctccatagaagacaccgggaccgatccagcctcgagaga | |
| | cccaatgctagccaccatgaattgtagagaattacccttgaccctttgggtgcttatatctgtaagcactgcagaatcttgt | |
| | acttcacgtccccacattactgtggttgaaggggaacctttctatctgaaacattgctcgtgttcacttgcacatgagattga | |
| | aacaaccaccaaaagctggtacaaaagcagtggatcacaggaacatgtggagctgaacccaaggagttccagcaga | |
| | attgctttgcatgattgtgttttggagttttggccagttgagttgaatgacacaggatcttactttttccaaatgaaaaattata | |
| | ctcagaaatggaaattaaatgtcatcagaagaaataaacacagctgtttcactgaaagacaagtaactagtaaaattgtg | |
| | gaagttaaaaaattttttcagataacctgtgaaaacagttactatcaaacactggtcaacagcacatcattgtataagaact | |
| | gtaaaaagctactactggagaacaataaaaacccaacgataaagaagaacgccgagtttgaagatcaggggtattact | |
| | cctgcgtgcatttccttcatcataatggaaaactatttaatatcaccaaaaccttcaatataacaatagtggaagatcgcagt | |
| | aatatagttccggttcttcttggaccaaagcttaaccatgttgcagtggaattaggaaaaaacgtaaggctcaactgctct | |
| | gctttgctgaatgaagaggatgtaatttattggatgttcggggaagaaaatggatcggatcctaatatacatgaagagaa | |
| | agaaatgagaattatgactccagaaggcaaatggcatgcttcaaaagtattgagaattgaaaatattggtgaaagcaatc | |
| | taaatgttttatataattgcactgtggccagcacgggaggcacagacaccaaaagcttcatcttggtgagaaaagcaga | |
| | catggctgatatcccaggccacgtcttcacaagaggaatgatcatagctgttttgatcttggtggcagtagtgtgcctagt | |
| | gactgtgtgtgtcatttatagagttgacttggttctattttatagacatttaacgagaagagatgaaacattaacagatggaa | |
| | aaacatatgatgcttttgtgtcttacctaaaagaatgccgacctgaaaatggagaggagcacacctttgctgtggagatttt | |
| | gcccagggtgttggagaaacattttgggtataagttatgcatatttgaaagggatgtagtgcctggaggagctgttgttga | |
| | tgaaatccactcactgatagagaaaagccgaagactaatcattgtcctaagtaaaagttatatgtctaatgaggtcaggta | |
| | tgaacttgaaagtggactccatgaagcattggtggaaagaaaaattaaaataatcttaattgaatttacacctgttactgac | |
| | ttcacattcttgccccaatcactaaagcttttgaaatctcacagagttctgaagtggaaggccgataaatctctttcttataac | |
| | tcaaggttctggaagaaccttctttacttaatgcctgcaaaaacagtcaagccaggtagagacgaaccggaagtcttgc | |
| | ctgttctttccgagtctggaagcggtgagggaagaggtagcctgctcacgtgcggtgatgtggaggagaaccctggg | |
| | cctatgctctgtttgggctggatatttctttggcttgttgcaggagagcgaattaaaggatttaatatttcaggttgttccaca | |
| | aaaaaactcctttggacatattctacaaggagtgaagaggaatttgtcttattttgtgatttaccagagccacagaaatcac | |
| | atttctgccacagaaatcgactctcaccaaaacaagtccctgagcacctgcccttcatgggtagtaacgacctatctgat | |
| | gtccaatggtaccaacaaccttcgaatggagatccattagaggacattaggaaaagctatcctcacatcattcaggaca | |
| | aatgtacccttcactttttgaccccaggggtgaataattctgggtcatatatttgtagacccaagatgattaagagcccctat | |
| | gatgtagcctgttgtgtcaagatgattttagaagttaagccccagacaaatgcatcctgtgagtattccgcatcacataag | |
| | caagacctacttcttgggagcactggctctatttcttgccccagtctcagctgccaaagtgatgcacaaagtccagcggt | |
| | aacctggtacaagaatggaaaactcctctctgtggaaaggagcaaccgaatcgtagtggatgaagtttatgactatcac | |
| | cagggcacatatgtatgtgattacactcagtcggatactgtgagttcgtggacagtcagagctgttgttcaagtgagaac | |
| | cattgtgggagacactaaactcaaaccagatattctggatcctgtcgaggacacactggaagtagaacttggaaagcct | |
| | ttaactattagctgcaaagcacgatttggctttgaaagggtctttaaccctgtcataaaatggtacatcaaagattctgacct | |
| | agagtgggaagtctcagtacctgaggcgaaaagtattaaatccactttaaaggatgaaatcattgagcgtaatatcatctt | |
| | ggaaaaagtcactcagcgtgatcttcgcaggaagtttgtttgctttgtccagaactccattggaaacacaacccagtccgt | |
| | ccaactgaaagaaaagagaggagtggtgctcctgtacatcctgcttggcaccatcgggaccctggtggccgtgctgg | |
| | cggcgagtgccctcctctacaggcactggattgaaatagtgctgctgtaccggacctaccagagcaaggatcagacg | |
| | cttggggataaaaaggattttgatgctttcgtatcctatgcaaaatggagctcttttccaagtgaggccacttcatctctgag | |
| | tgaagaacacttggccctgagcctatttcctgatgttttagaaaacaaatatggatatagcctgtgtttgcttgaaagagat | |
| | gtggctccaggaggagtgtatgcagaagacattgtgagcattattaagagaagcagaagaggaatatttatcttgagcc | |
| | ccaactatgtcaatggacccagtatctttgaactacaagcagcagtgaatcttgccttggatgatcaaacactgaaactca | |
| | ttttaattaagttctgttacttccaagagccagagtctctacctcatctcgtgaaaaaagctctcagggttttgcccacagtta | |
| | cttggagaggcttaaaatcagttcctcccaattctaggttctgggccaaaatgcgctaccacatgcctgtgaaaaactctc | |
| | agggattcacgtggaaccagctcagaattacctctaggatttttcagtggaaaggactcagtagaacagaaaccactgg | |
| | gaggagctcccagcctaaggaatggtgataa | |
| CAR E6 CD8- | atggccttaccagtgaccgccttgctcctgccgctggccttgctgctccacgccgccaggccggaggtgcagctggtg | 181 |
| CD8-41BB1XX | gagtctgggggaggcctggtcaagcctggggggtccctgagactctcctgtgcagcctctggattcaccttcagtagg | |
| 3XFOXP3NFAT | tatggcatgagctgggtccgccaggctccagggaagaggctggagtgggtctcaaccattagtggcggaggcacct | |
| mCMV | acatatactacccagactcagtgaagggccgattcaccatctccagagacaacgccaagaacaccctgtatctgcaaat | |
| IL18Ralpha-T2A- | gaacagcctgagagccgaggacacggctgtgtattactgtaccagagataactatggccgcaactatgattatggcatg | |
| IL18Rb | gattattggggccagggcaccctggtgaccgtgagcagcggtggtggcggttccggcggtggcggttccggtggtg | |
| | gcggttccgaaattgtgttgacacagtctccagccaccctgtctttgtctccaggggaaagagccaccctcacctgcag | |
| | cgccaccagcagtgttagctacatccactggtatcaacagaggcctggccagagccccaggctcctcatctatagcac | |
| | ctccaacctggccagcggcatcccagccaggttcagtggcagtgggtctgggagcgactacactctcaccatcagca | |
| | gcctagagcctgaagattttgcagtttattactgtcagcagcgtagcagctcccctttcacctttggcagcggcaccaaa | |
| | gtggaaattaaaacaacaacccctgcccccagacctcctaccccagcccctacaattgccagccagcctctgagcctg | |
| | aggcccgaggcttgtagacctgctgctggcggagccgtgcacaccagaggactggatttcgcctgcgacatctacatc | |
| | tgggcgcccttggccgggacttgtggggtccttctcctgtcactggttatcaccctttactgcaaacggggcagaaaga | |
| | aactcctgtatatattcaaacaaccatttatgagaccagtacaaactactcaagaggaagatggctgtagctgccgatttc | |
| | cagaagaagaagaaggaggatgtgaactgagagtgaagttcagcaggagcgcagacgcccccgcgtacaagcag | |
| | ggccagaaccagctctataacgagctcaatctaggacgaagagaggagtacgatgttttggacaagagacgtggccg | |
| | ggaccctgagatggggggaaagccgagaaggaagaaccctcaggaaggcctgttcaatgaactgcagaaagataa | |
| | gatggcggaggccttcagtgagattgggatgaaaggcgagcgccggaggggcaaggggcacgatggccttttcca | |
| | gggtctcagtacagccaccaaggacaccttcgacgcccttcacatgcaggccctgccccctcgctgataagtttaaact | |
| | gccagaacatttctctggcctaactggccggtaccggcttcattttttccatttactgcagaggcttcattttttccatttactg | |
| | cagaggcttcattttttccatttactgcagaactagttaggcgtgtacggtgggaggcctatataagcagagctcgtttagt | |
| | gaaccgtcagatcgcctggagacgccatccacgctgttttgacctccatagaagacaccgggaccgatccagcctcg | |
| | agagacccaatgctagccaccatgaattgtagagaattacccttgaccctttgggtgcttatatctgtaagcactgcaga | |
| | atcttgtacttcacgtccccacattactgtggttgaaggggaacctttctatctgaaacattgctcgtgttcacttgcacatg | |
| | agattgaaacaaccaccaaaagctggtacaaaagcagtggatcacaggaacatgtggagctgaacccaaggagttcc | |
| | agcagaattgctttgcatgattgtgttttggagttttggccagttgagttgaatgacacaggatcttactttttccaaatgaaa | |
| | aattatactcagaaatggaaattaaatgtcatcagaagaaataaacacagctgtttcactgaaagacaagtaactagtaaa | |
| | attgtggaagttaaaaaattttttcagataacctgtgaaaacagttactatcaaacactggtcaacagcacatcattgtataa | |
| | gaactgtaaaaagctactactggagaacaataaaaacccaacgataaagaagaacgccgagtttgaagatcaggggt | |
| | attactcctgcgtgcatttccttcatcataatggaaaactatttaatatcaccaaaaccttcaatataacaatagtggaagatc | |
| | gcagtaatatagttccggttcttcttggaccaaagcttaaccatgttgcagtggaattaggaaaaaacgtaaggctcaact | |
| | gctctgctttgctgaatgaagaggatgtaatttattggatgttcggggaagaaaatggatcggatcctaatatacatgaag | |
| | agaaagaaatgagaattatgactccagaaggcaaatggcatgcttcaaaagtattgagaattgaaaatattggtgaaag | |
| | caatctaaatgttttatataattgcactgtggccagcacgggaggcacagacaccaaaagcttcatcttggtgagaaaag | |
| | cagacatggctgatatcccaggccacgtcttcacaagaggaatgatcatagctgttttgatcttggtggcagtagtgtgc | |
| | ctagtgactgtgtgtgtcatttatagagttgacttggttctattttatagacatttaacgagaagagatgaaacattaacagat | |
| | ggaaaaacatatgatgcttttgtgtcttacctaaaagaatgccgacctgaaaatggagaggagcacacctttgctgtgga | |
| | gattttgcccagggtgttggagaaacattttgggtataagttatgcatatttgaaagggatgtagtgcctggaggagctgtt | |
| | gttgatgaaatccactcactgatagagaaaagccgaagactaatcattgtcctaagtaaaagttatatgtctaatgaggtc | |
| | aggtatgaacttgaaagtggactccatgaagcattggtggaaagaaaaattaaaataatcttaattgaatttacacctgtta | |
| | ctgacttcacattcttgccccaatcactaaagcttttgaaatctcacagagttctgaagtggaaggccgataaatctctttct | |
| | tataactcaaggttctggaagaaccttctttacttaatgcctgcaaaaacagtcaagccaggtagagacgaaccggaagt | |
| | cttgcctgttctttccgagtctggaagcggtgagggaagaggtagcctgctcacgtgcggtgatgtggaggagaaccc | |
| | tgggcctatgctctgtttgggctggatatttctttggcttgttgcaggagagcgaattaaaggatttaatatttcaggttgttc | |
| | cacaaaaaaactcctttggacatattctacaaggagtgaagaggaatttgtcttattttgtgatttaccagagccacagaaa | |
| | tcacatttctgccacagaaatcgactctcaccaaaacaagtccctgagcacctgcccttcatgggtagtaacgacctatct | |
| | gatgtccaatggtaccaacaaccttcgaatggagatccattagaggacattaggaaaagctatcctcacatcattcagga | |
| | caaatgtacccttcactttttgaccccaggggtgaataattctgggtcatatatttgtagacccaagatgattaagagcccc | |
| | tatgatgtagcctgttgtgtcaagatgattttagaagttaagccccagacaaatgcatcctgtgagtattccgcatcacata | |
| | agcaagacctacttcttgggagcactggctctatttcttgccccagtctcagctgccaaagtgatgcacaaagtccagcg | |
| | gtaacctggtacaagaatggaaaactcctctctgtggaaaggagcaaccgaatcgtagtggatgaagtttatgactatca | |
| | ccagggcacatatgtatgtgattacactcagtcggatactgtgagttcgtggacagtcagagctgttgttcaagtgagaa | |
| | ccattgtgggagacactaaactcaaaccagatattctggatcctgtcgaggacacactggaagtagaacttggaaagcc | |
| | tttaactattagctgcaaagcacgatttggctttgaaagggtctttaaccctgtcataaaatggtacatcaaagattctgacc | |
| | tagagtgggaagtctcagtacctgaggcgaaaagtattaaatccactttaaaggatgaaatcattgagcgtaatatcatct | |
| | tggaaaaagtcactcagcgtgatcttcgcaggaagtttgtttgctttgtccagaactccattggaaacacaacccagtccg | |
| | tccaactgaaagaaaagagaggagtggtgctcctgtacatcctgcttggcaccatcgggaccctggtggccgtgctgg | |
| | cggcgagtgccctcctctacaggcactggattgaaatagtgctgctgtaccggacctaccagagcaaggatcagacg | |
| | cttggggataaaaaggattttgatgctttcgtatcctatgcaaaatggagctcttttccaagtgaggccacttcatctctgag | |
| | tgaagaacacttggccctgagcctatttcctgatgttttagaaaacaaatatggatatagcctgtgtttgcttgaaagagat | |
| | gtggctccaggaggagtgtatgcagaagacattgtgagcattattaagagaagcagaagaggaatatttatcttgagcc | |
| | ccaactatgtcaatggacccagtatctttgaactacaagcagcagtgaatcttgccttggatgatcaaacactgaaactca | |
| | ttttaattaagttctgttacttccaagagccagagtctctacctcatctcgtgaaaaaagctctcagggttttgcccacagtta | |
| | cttggagaggcttaaaatcagttcctcccaattctaggttctgggccaaaatgcgctaccacatgcctgtgaaaaactctc | |
| | agggattcacgtggaaccagctcagaattacctctaggatttttcagtggaaaggactcagtagaacagaaaccactgg | |
| | gaggagctcccagcctaaggaatggtgataa | |
| CAR E6 CD8- | atggccttaccagtgaccgccttgctcctgccgctggccttgctgctccacgccgccaggccggaggtgcagctggtg | 182 |
| CD8-41BB1XX | gagtctgggggaggcctggtcaagcctggggggtccctgagactctcctgtgcagcctctggattcaccttcagtagg | |
| 6XIL12NFAT | tatggcatgagctgggtccgccaggctccagggaagaggctggagtgggtctcaaccattagtggcggaggcacct | |
| mCMV | acatatactacccagactcagtgaagggccgattcaccatctccagagacaacgccaagaacaccctgtatctgcaaat | |
| IL18Ralpha-T2A- | gaacagcctgagagccgaggacacggctgtgtattactgtaccagagataactatggccgcaactatgattatggcatg | |
| IL18Rb | gattattggggccagggcaccctggtgaccgtgagcagcggtggtggcggttccggcggtggcggttccggtggtg | |
| | gcggttccgaaattgtgttgacacagtctccagccaccctgtctttgtctccaggggaaagagccaccctcacctgcag | |
| | cgccaccagcagtgttagctacatccactggtatcaacagaggcctggccagagccccaggctcctcatctatagcac | |
| | ctccaacctggccagcggcatcccagccaggttcagtggcagtgggtctgggagcgactacactctcaccatcagca | |
| | gcctagagcctgaagattttgcagtttattactgtcagcagcgtagcagctcccctttcacctttggcagcggcaccaaa | |
| | gtggaaattaaaacaacaacccctgcccccagacctcctaccccagcccctacaattgccagccagcctctgagcctg | |
| | aggcccgaggcttgtagacctgctgctggcggagccgtgcacaccagaggactggatttcgcctgcgacatctacatc | |
| | tgggcgcccttggccgggacttgtggggtccttctcctgtcactggttatcaccctttactgcaaacggggcagaaaga | |
| | aactcctgtatatattcaaacaaccatttatgagaccagtacaaactactcaagaggaagatggctgtagctgccgatttc | |
| | cagaagaagaagaaggaggatgtgaactgagagtgaagttcagcaggagcgcagacgcccccgcgtacaagcag | |
| | ggccagaaccagctctataacgagctcaatctaggacgaagagaggagtacgatgttttggacaagagacgtggccg | |
| | ggaccctgagatggggggaaagccgagaaggaagaaccctcaggaaggcctgttcaatgaactgcagaaagataa | |
| | gatggcggaggccttcagtgagattgggatgaaaggcgagcgccggaggggcaaggggcacgatggccttttcca | |
| | gggtctcagtacagccaccaaggacaccttcgacgcccttcacatgcaggccctgccccctcgctgataagtttaaact | |
| | gccagaacatttctctggcctaactggccggtaccggaggaaaaactgtttcatacagaaggcgtggaggaaaaactg | |
| | tttcatacagaaggcgtggaggaaaaactgtttcatacagaaggcgtggaggaaaaactgtttcatacagaaggcgtg | |
| | gaggaaaaactgtttcatacagaaggcgtggaggaaaaactgtttcatacagaaggcgtactagttaggcgtgtacggt | |
| | gggaggcctatataagcagagctcgtttagtgaaccgtcagatcgcctggagacgccatccacgctgttttgacctcca | |
| | tagaagacaccgggaccgatccagcctcgagagacccaatgctagccaccatgaattgtagagaattacccttgacc | |
| | ctttgggtgcttatatctgtaagcactgcagaatcttgtacttcacgtccccacattactgtggttgaaggggaacctttctat | |
| | ctgaaacattgctcgtgttcacttgcacatgagattgaaacaaccaccaaaagctggtacaaaagcagtggatcacagg | |
| | aacatgtggagctgaacccaaggagttccagcagaattgctttgcatgattgtgttttggagttttggccagttgagttgaa | |
| | tgacacaggatcttactttttccaaatgaaaaattatactcagaaatggaaattaaatgtcatcagaagaaataaacacag | |
| | ctgtttcactgaaagacaagtaactagtaaaattgtggaagttaaaaaattttttcagataacctgtgaaaacagttactatc | |
| | aaacactggtcaacagcacatcattgtataagaactgtaaaaagctactactggagaacaataaaaacccaacgataaa | |
| | gaagaacgccgagtttgaagatcaggggtattactcctgcgtgcatttccttcatcataatggaaaactatttaatatcacc | |
| | aaaaccttcaatataacaatagtggaagatcgcagtaatatagttccggttcttcttggaccaaagcttaaccatgttgcag | |
| | tggaattaggaaaaaacgtaaggctcaactgctctgctttgctgaatgaagaggatgtaatttattggatgttcggggaa | |
| | gaaaatggatcggatcctaatatacatgaagagaaagaaatgagaattatgactccagaaggcaaatggcatgcttcaa | |
| | aagtattgagaattgaaaatattggtgaaagcaatctaaatgttttatataattgcactgtggccagcacgggaggcacag | |
| | acaccaaaagcttcatcttggtgagaaaagcagacatggctgatatcccaggccacgtcttcacaagaggaatgatcat | |
| | agctgttttgatcttggtggcagtagtgtgcctagtgactgtgtgtgtcatttatagagttgacttggttctattttatagacatt | |
| | taacgagaagagatgaaacattaacagatggaaaaacatatgatgcttttgtgtcttacctaaaagaatgccgacctgaa | |
| | aatggagaggagcacacctttgctgtggagattttgcccagggtgttggagaaacattttgggtataagttatgcatattt | |
| | gaaagggatgtagtgcctggaggagctgttgttgatgaaatccactcactgatagagaaaagccgaagactaatcattg | |
| | tcctaagtaaaagttatatgtctaatgaggtcaggtatgaacttgaaagtggactccatgaagcattggtggaaagaaaa | |
| | attaaaataatcttaattgaatttacacctgttactgacttcacattcttgccccaatcactaaagcttttgaaatctcacagag | |
| | ttctgaagtggaaggccgataaatctctttcttataactcaaggttctggaagaaccttctttacttaatgcctgcaaaaaca | |
| | gtcaagccaggtagagacgaaccggaagtcttgcctgttctttccgagtctggaagcggtgagggaagaggtagcct | |
| | gctcacgtgcggtgatgtggaggagaaccctgggcctatgctctgtttgggctggatatttctttggcttgttgcaggaga | |
| | gcgaattaaaggatttaatatttcaggttgttccacaaaaaaactcctttggacatattctacaaggagtgaagaggaattt | |
| | gtcttattttgtgatttaccagagccacagaaatcacatttctgccacagaaatcgactctcaccaaaacaagtccctgag | |
| | cacctgcccttcatgggtagtaacgacctatctgatgtccaatggtaccaacaaccttcgaatggagatccattagagga | |
| | cattaggaaaagctatcctcacatcattcaggacaaatgtacccttcactttttgaccccaggggtgaataattctgggtca | |
| | tatatttgtagacccaagatgattaagagcccctatgatgtagcctgttgtgtcaagatgattttagaagttaagccccaga | |
| | caaatgcatcctgtgagtattccgcatcacataagcaagacctacttcttgggagcactggctctatttcttgccccagtct | |
| | cagctgccaaagtgatgcacaaagtccagcggtaacctggtacaagaatggaaaactcctctctgtggaaaggagca | |
| | accgaatcgtagtggatgaagtttatgactatcaccagggcacatatgtatgtgattacactcagtcggatactgtgagtt | |
| | cgtggacagtcagagctgttgttcaagtgagaaccattgtgggagacactaaactcaaaccagatattctggatcctgtc | |
| | gaggacacactggaagtagaacttggaaagcctttaactattagctgcaaagcacgatttggctttgaaagggtctttaa | |
| | ccctgtcataaaatggtacatcaaagattctgacctagagtgggaagtctcagtacctgaggcgaaaagtattaaatcca | |
| | ctttaaaggatgaaatcattgagcgtaatatcatcttggaaaaagtcactcagcgtgatcttcgcaggaagtttgtttgcttt | |
| | gtccagaactccattggaaacacaacccagtccgtccaactgaaagaaaagagaggagtggtgctcctgtacatcctg | |
| | cttggcaccatcgggaccctggtggccgtgctggcggcgagtgccctcctctacaggcactggattgaaatagtgctg | |
| | ctgtaccggacctaccagagcaaggatcagacgcttggggataaaaaggattttgatgctttcgtatcctatgcaaaatg | |
| | gagctcttttccaagtgaggccacttcatctctgagtgaagaacacttggccctgagcctatttcctgatgttttagaaaac | |
| | aaatatggatatagcctgtgtttgcttgaaagagatgtggctccaggaggagtgtatgcagaagacattgtgagcattatt | |
| | aagagaagcagaagaggaatatttatcttgagccccaactatgtcaatggacccagtatctttgaactacaagcagcagt | |
| | gaatcttgccttggatgatcaaacactgaaactcattttaattaagttctgttacttccaagagccagagtctctacctcatct | |
| | cgtgaaaaaagctctcagggttttgcccacagttacttggagaggcttaaaatcagttcctcccaattctaggttctgggc | |
| | caaaatgcgctaccacatgcctgtgaaaaactctcagggattcacgtggaaccagctcagaattacctctaggatttttca | |
| | gtggaaaggactcagtagaacagaaaccactgggaggagctcccagcctaaggaatggtgataa | |
| CAR E6 CD8- | atggccttaccagtgaccgccttgctcctgccgctggccttgctgctccacgccgccaggccggaggtgcagctggtg | 183 |
| CD8-41BB1XX | gagtctgggggaggcctggtcaagcctggggggtccctgagactctcctgtgcagcctctggattcaccttcagtagg | |
| 3XIL12NFAT | tatggcatgagctgggtccgccaggctccagggaagaggctggagtgggtctcaaccattagtggcggaggcacct | |
| mCMV | acatatactacccagactcagtgaagggccgattcaccatctccagagacaacgccaagaacaccctgtatctgcaaat | |
| IL18Ralpha-T2A- | gaacagcctgagagccgaggacacggctgtgtattactgtaccagagataactatggccgcaactatgattatggcatg | |
| IL18Rb | gattattggggccagggcaccctggtgaccgtgagcagcggtggtggcggttccggcggtggcggttccggtggtg | |
| | gcggttccgaaattgtgttgacacagtctccagccaccctgtctttgtctccaggggaaagagccaccctcacctgcag | |
| | cgccaccagcagtgttagctacatccactggtatcaacagaggcctggccagagccccaggctcctcatctatagcac | |
| | ctccaacctggccagcggcatcccagccaggttcagtggcagtgggtctgggagcgactacactctcaccatcagca | |
| | gcctagagcctgaagattttgcagtttattactgtcagcagcgtagcagctcccctttcacctttggcagcggcaccaaa | |
| | gtggaaattaaaacaacaacccctgcccccagacctcctaccccagcccctacaattgccagccagcctctgagcctg | |
| | aggcccgaggcttgtagacctgctgctggcggagccgtgcacaccagaggactggatttcgcctgcgacatctacatc | |
| | tgggcgcccttggccgggacttgtggggtccttctcctgtcactggttatcaccctttactgcaaacggggcagaaaga | |
| | aactcctgtatatattcaaacaaccatttatgagaccagtacaaactactcaagaggaagatggctgtagctgccgatttc | |
| | cagaagaagaagaaggaggatgtgaactgagagtgaagttcagcaggagcgcagacgcccccgcgtacaagcag | |
| | ggccagaaccagctctataacgagctcaatctaggacgaagagaggagtacgatgttttggacaagagacgtggccg | |
| | ggaccctgagatggggggaaagccgagaaggaagaaccctcaggaaggcctgttcaatgaactgcagaaagataa | |
| | gatggcggaggccttcagtgagattgggatgaaaggcgagcgccggaggggcaaggggcacgatggccttttcca | |
| | gggtctcagtacagccaccaaggacaccttcgacgcccttcacatgcaggccctgccccctcgctgataagtttaaact | |
| | gccagaacatttctctggcctaactggccggtaccggaggaaaaactgtttcatacagaaggcgtggaggaaaaactg | |
| | tttcatacagaaggcgtggaggaaaaactgtttcatacagaaggcgtactagttaggcgtgtacggtgggaggcctata | |
| | taagcagagctcgtttagtgaaccgtcagatcgcctggagacgccatccacgctgttttgacctccatagaagacaccg | |
| | ggaccgatccagcctcgagagacccaatgctagccaccatgaattgtagagaattacccttgaccctttgggtgcttat | |
| | atctgtaagcactgcagaatcttgtacttcacgtccccacattactgtggttgaaggggaacctttctatctgaaacattgct | |
| | cgtgttcacttgcacatgagattgaaacaaccaccaaaagctggtacaaaagcagtggatcacaggaacatgtggagc | |
| | tgaacccaaggagttccagcagaattgctttgcatgattgtgttttggagttttggccagttgagttgaatgacacaggatc | |
| | ttactttttccaaatgaaaaattatactcagaaatggaaattaaatgtcatcagaagaaataaacacagctgtttcactgaaa | |
| | gacaagtaactagtaaaattgtggaagttaaaaaattttttcagataacctgtgaaaacagttactatcaaacactggtcaa | |
| | cagcacatcattgtataagaactgtaaaaagctactactggagaacaataaaaacccaacgataaagaagaacgccga | |
| | gtttgaagatcaggggtattactcctgcgtgcatttccttcatcataatggaaaactatttaatatcaccaaaaccttcaatat | |
| | aacaatagtggaagatcgcagtaatatagttccggttcttcttggaccaaagcttaaccatgttgcagtggaattaggaaa | |
| | aaacgtaaggctcaactgctctgctttgctgaatgaagaggatgtaatttattggatgttcggggaagaaaatggatc gg | |
| | atcctaatatacatgaagagaaagaaatgagaattatgactccagaaggcaaatggcatgcttcaaaagtattgagaatt | |
| | gaaaatattggtgaaagcaatctaaatgttttatataattgcactgtggccagcacgggaggcacagacaccaaaagctt | |
| | catcttggtgagaaaagcagacatggctgatatcccaggccacgtcttcacaagaggaatgatcatagctgttttgatctt | |
| | ggtggcagtagtgtgcctagtgactgtgtgtgtcatttatagagttgacttggttctattttatagacatttaacgagaagag | |
| | atgaaacattaacagatggaaaaacatatgatgcttttgtgtcttacctaaaagaatgccgacctgaaaatggagaggag | |
| | cacacctttgctgtggagattttgcccagggtgttggagaaacattttgggtataagttatgcatatttgaaagggatgtag | |
| | tgcctggaggagctgttgttgatgaaatccactcactgatagagaaaagccgaagactaatcattgtcctaagtaaaagt | |
| | tatatgtctaatgaggtcaggtatgaacttgaaagtggactccatgaagcattggtggaaagaaaaattaaaataatctta | |
| | attgaatttacacctgttactgacttcacattcttgccccaatcactaaagcttttgaaatctcacagagttctgaagtggaa | |
| | ggccgataaatctctttcttataactcaaggttctggaagaaccttctttacttaatgcctgcaaaaacagtcaagccaggt | |
| | agagacgaaccggaagtcttgcctgttctttccgagtctggaagcggtgagggaagaggtagcctgctcacgtgcggt | |
| | gatgtggaggagaaccctgggcctatgctctgtttgggctggatatttctttggcttgttgcaggagagcgaattaaagg | |
| | atttaatatttcaggttgttccacaaaaaaactcctttggacatattctacaaggagtgaagaggaatttgtcttattttgtgat | |
| | ttaccagagccacagaaatcacatttctgccacagaaatcgactctcaccaaaacaagtccctgagcacctgcccttcat | |
| | gggtagtaacgacctatctgatgtccaatggtaccaacaaccttcgaatggagatccattagaggacattaggaaaagc | |
| | tatcctcacatcattcaggacaaatgtacccttcactttttgaccccaggggtgaataattctgggtcatatatttgtagacc | |
| | caagatgattaagagcccctatgatgtagcctgttgtgtcaagatgattttagaagttaagccccagacaaatgcatcctg | |
| | tgagtattccgcatcacataagcaagacctacttcttgggagcactggctctatttcttgccccagtctcagctgccaaagt | |
| | gatgcacaaagtccagcggtaacctggtacaagaatggaaaactcctctctgtggaaaggagcaaccgaatcgtagtg | |
| | gatgaagtttatgactatcaccagggcacatatgtatgtgattacactcagtcggatactgtgagttcgtggacagtcaga | |
| | gctgttgttcaagtgagaaccattgtgggagacactaaactcaaaccagatattctggatcctgtcgaggacacactgga | |
| | agtagaacttggaaagcctttaactattagctgcaaagcacgatttggctttgaaagggtctttaaccctgtcataaaatgg | |
| | tacatcaaagattctgacctagagtgggaagtctcagtacctgaggcgaaaagtattaaatccactttaaaggatgaaat | |
| | cattgagcgtaatatcatcttggaaaaagtcactcagcgtgatcttcgcaggaagtttgtttgctttgtccagaactccattg | |
| | gaaacacaacccagtccgtccaactgaaagaaaagagaggagtggtgctcctgtacatcctgcttggcaccatcggg | |
| | accctggtggccgtgctggcggcgagtgccctcctctacaggcactggattgaaatagtgctgctgtaccggacctac | |
| | cagagcaaggatcagacgcttggggataaaaaggattttgatgctttcgtatcctatgcaaaatggagctcttttccaagt | |
| | gaggccacttcatctctgagtgaagaacacttggccctgagcctatttcctgatgttttagaaaacaaatatggatatagc | |
| | ctgtgtttgcttgaaagagatgtggctccaggaggagtgtatgcagaagacattgtgagcattattaagagaagcagaa | |
| | gaggaatatttatcttgagccccaactatgtcaatggacccagtatctttgaactacaagcagcagtgaatcttgccttgg | |
| | atgatcaaacactgaaactcattttaattaagttctgttacttccaagagccagagtctctacctcatctc gtgaaaaaagct | |
| | ctcagggttttgcccacagttacttggagaggcttaaaatcagttcctcccaattctaggttctgggccaaaatgcgctac | |
| | cacatgcctgtgaaaaactctcagggattcacgtggaaccagctcagaattacctctaggatttttcagtggaaaggact | |
| | cagtagaacagaaaccactgggaggagctcccagcctaaggaatggtgataa | |
| CAR 20A10N | atggccttaccagtgaccgccttgctcctgccgctggccttgctgctccacgccgccaggccgcaggtgcagctggtt | 184 |
| CD8-CD8-41BB | gaatctggcggcggacttgtgaagcctggcggatctctgagactgagctgtgccgccagcggcttcacctttagcacat | |
| 1XX | acgccatgagctggatcagacaggcccctggcaaaggcctggaatgggtggcgtctattggcagagccggcagcac | |
| 6XFOXP3NFAT | ctactacagcgactctgtgaagggcagattcaccatcagccgggacaacgccaagaacagcctgtacctgcagatga | |
| mCMV | actccctgagagccgaggacaccgccgtgtactattgtgccagaggacccatctacaacgactacgacgagttcgcct | |
| IL18Ralpha | attggggccagggcacactggtcacagtttctagcggcggtggcggaagcggaggcggtggctccggtggcggag | |
| | gcagcgaaattgtgctgacacagagccccgccacactgtcactttctccaggcgaaagagccacactgagctgcaag | |
| | agcagccagagcgtgctgtactccagcaaccagaagaactacctggcctggtatcagcagaagcccggccaagctc | |
| | ctcggctgctgatctattgggccagcacaagagagagcggcatccctgccagattttctggcagcggctctggcaccg | |
| | atttcaccctgaccataagcagcctggaacctgaggacttcgccgtgtattactgccaccagtacctgagcagcctgac | |
| | ctttggcggaggcaccaaggtggaaatcaagcggacaacaacccctgcccccagacctcctaccccagcccctaca | |
| | attgccagccagcctctgagcctgaggcccgaggcttgtagacctgctgctggcggagccgtgcacaccagaggact | |
| | ggatttcgcctgcgacatctacatctgggcgcccttggccgggacttgtggggtccttctcctgtcactggttatcaccct | |
| | ttactgcaaacggggcagaaagaaactcctgtatatattcaaacaaccatttatgagaccagtacaaactactcaagagg | |
| | aagatggctgtagctgccgatttccagaagaagaagaaggaggatgtgaactgagagtgaagttcagcaggagcgc | |
| | agacgcccccgcgtacaagcagggccagaaccagctctataacgagctcaatctaggacgaagagaggagtacgat | |
| | gttttggacaagagacgtggccgggaccctgagatggggggaaagccgagaaggaagaaccctcaggaaggcctg | |
| | ttcaatgaactgcagaaagataagatggcggaggccttcagtgagattgggatgaaaggcgagcgccggaggggca | |
| | aggggcacgatggccttttccagggtctcagtacagccaccaaggacaccttcgacgcccttcacatgcaggccctgc | |
| | cccctcgctgataagtttaaactgccagaacatttctctggcctaactggccggtaccggcttcattttttccatttactgca | |
| | gaggcttcattttttccatttactgcagaggcttcattttttccatttactgcagaggcttcattttttccatttactgcagaggct | |
| | tcattttttccatttactgcagaggcttcattttttccatttactgcagaactagttaggcgtgtacggtgggaggcctatataa | |
| | gcagagctcgtttagtgaaccgtcagatcgcctggagacgccatccacgctgttttgacctccatagaagacaccggg | |
| | accgatccagcctcgagagacccaatgctagccaccatgaattgtagagaattacccttgaccctttgggtgcttatatc | |
| | tgtaagcactgcagaatcttgtacttcacgtccccacattactgtggttgaaggggaacctttctatctgaaacattgctcgt | |
| | gttcacttgcacatgagattgaaacaaccaccaaaagctggtacaaaagcagtggatcacaggaacatgtggagctga | |
| | acccaaggagttccagcagaattgctttgcatgattgtgttttggagttttggccagttgagttgaatgacacaggatctta | |
| | ctttttccaaatgaaaaattatactcagaaatggaaattaaatgtcatcagaagaaataaacacagtgtttcactgaaaga | |
| | caagtaactagtaaaattgtggaagttaaaaaattttttcagataacctgtgaaaacagttactatcaaacactggtcaaca | |
| | gcacatcattgtataagaactgtaaaaagctactactggagaacaataaaaacccaacgataaagaagaacgccgagt | |
| | ttgaagatcaggggtattactcctgcgtgcatttccttcatcataatggaaaactatttaatatcaccaaaaccttcaatataa | |
| | caatagtggaagatcgcagtaatatagttccggttcttcttggaccaaagcttaaccatgttgcagtggaattaggaaaaa | |
| | acgtaaggctcaactgctctgctttgctgaatgaagaggatgtaatttattggatgttcggggaagaaaatggatcggat | |
| | cctaatatacatgaagagaaagaaatgagaattatgactccagaaggcaaatggcatgcttcaaaagtattgagaattga | |
| | aaatattggtgaaagcaatctaaatgttttatataattgcactgtggccagcacgggaggcacagacaccaaaagcttca | |
| | tcttggtgagaaaagcagacatggctgatatcccaggccacgtcttcacaagaggaatgatcatagctgttttgatcttgg | |
| | tggcagtagtgtgcctagtgactgtgtgtgtcatttatagagttgacttggttctattttatagacatttaacgagaagagatg | |
| | aaacattaacagatggaaaaacatatgatgcttttgtgtcttacctaaaagaatgccgacctgaaaatggagaggagcac | |
| | acctttgctgtggagattttgcccagggtgttggagaaacattttgggtataagttatgcatatttgaaagggatgtagtgc | |
| | ctggaggagctgttgttgatgaaatccactcactgatagagaaaagccgaagactaatcattgtcctaagtaaaagttata | |
| | tgtctaatgaggtcaggtatgaacttgaaagtggactccatgaagcattggtggaaagaaaaattaaaataatcttaattg | |
| | aatttacacctgttactgacttcacattcttgccccaatcactaaagcttttgaaatctcacagagttctgaagtggaaggcc | |
| | gataaatctctttcttataactcaaggttctggaagaaccttctttacttaatgcctgcaaaaacagtcaagccaggtagag | |
| | acgaaccggaagtcttgcctgttctttccgagtcttgataa | |
| CAR 20A10N | atggccttaccagtgaccgccttgctcctgccgctggccttgctgctccacgccgccaggccgcaggtgcagctggtt | 185 |
| CD8-CD8-41BB | gaatctggcggcggacttgtgaagcctggcggatctctgagactgagctgtgccgccagggcttcacctttagcacat | |
| 1XX | acgccatgagctggatcagacaggcccctggcaaaggcctggaatgggggcgtctattggcagagccggcagcac | |
| 3XFOXP3NFAT | ctactacagcgactctgtgaagggcagattcaccatcagccgggacaacgccaagaacagcctgtacctgcagatga | |
| mCMV | actccctgagagccgaggacaccgccgtgtactattgtgccagaggacccatctacaacgactacgacgagttcgcct | |
| IL18Ralpha | attggggccagggcacactggtcacagtttctagcggcggtggcggaagcggaggcggtggctccggtggcggag | |
| | gcagcgaaattgtgctgacacagagccccgccacactgtcactttctccaggcgaaagagccacactgagctgcaag | |
| | agcagccagagcgtgctgtactccagcaaccagaagaactacctggcctggtatcagcagaagcccggccaagctc | |
| | ctcggctgctgatctattgggccagcacaagagagagcggcatccctgccagattttctggcagcggctctggcaccg | |
| | atttcaccctgaccataagcagcctggaacctgaggacttcgccgtgtattactgccaccagtacctgagcagcctgac | |
| | ctttggcggaggcaccaaggtggaaatcaagcggacaacaacccctgcccccagacctcctaccccagcccctaca | |
| | attgccagccagcctctgagcctgaggcccgaggcttgtagacctgctgctggcggagccgtgcacaccagaggact | |
| | ggatttcgcctgcgacatctacatctgggcgcccttggccgggacttgtggggtccttctcctgtcactggttatcaccct | |
| | ttactgcaaacggggcagaaagaaactcctgtatatattcaaacaaccatttatgagaccagtacaaactactcaagagg | |
| | aagatggctgtagctgccgatttccagaagaagaagaaggaggatgtgaactgagagtgaagttcagcaggagcgc | |
| | agacgcccccgcgtacaagcagggccagaaccagctctataacgagctcaatctaggacgaagagaggagtacgat | |
| | gttttggacaagagacgtggccgggaccctgagatggggggaaagccgagaaggaagaaccctcaggaaggcctg | |
| | ttcaatgaactgcagaaagataagatggcggaggccttcagtgagattgggatgaaaggcgagcgccggaggggca | |
| | aggggcacgatggccttttccagggtctcagtacagccaccaaggacaccttcgacgcccttcacatgcaggccctgc | |
| | cccctcgctgataagtttaaactgccagaacatttctctggcctaactggccggtaccggcttcattttttccatttactgca | |
| | gaggcttcattttttccatttactgcagaggcttcattttttccatttactgcagaactagttaggcgtgtacggtgggaggc | |
| | ctatataagcagagctcgtttagtgaaccgtcagatcgcctggagacgccatccacgctgttttgacctccatagaagac | |
| | accgggaccgatccagcctcgagagacccaatgctagccaccatgaattgtagagaattacccttgaccctttgggtg | |
| | cttatatctgtaagcactgcagaatcttgtacttcacgtccccacattactgtggttgaaggggaacctttctatctgaaaca | |
| | ttgctcgtgttcacttgcacatgagattgaaacaaccaccaaaagctggtacaaaagcagtggatcacaggaacatgtg | |
| | gagctgaacccaaggagttccagcagaattgctttgcatgattgtgttttggagttttggccagttgagttgaatgacaca | |
| | ggatcttactttttccaaatgaaaaattatactcagaaatggaaattaaatgtcatcagaagaaataaacacagctgtttcac | |
| | tgaaagacaagtaactagtaaaattgtggaagttaaaaaattttttcagataacctgtgaaaacagttactatcaaacactg | |
| | gtcaacagcacatcattgtataagaactgtaaaaagctactactggagaacaataaaaacccaacgataaagaagaac | |
| | gccgagtttgaagatcaggggtattactcctgcgtgcatttccttcatcataatggaaaactatttaatatcaccaaaacctt | |
| | caatataacaatagtggaagatcgcagtaatatagttccggttcttcttggaccaaagcttaaccatgttgcagtggaatta | |
| | ggaaaaaacgtaaggctcaactgctctgctttgctgaatgaagaggatgtaatttattggatgttcggggaagaaaatgg | |
| | atcggatcctaatatacatgaagagaaagaaatgagaattatgactccagaaggcaaatggcatgcttcaaaagtattga | |
| | gaattgaaaatattggtgaaagcaatctaaatgttttatataattgcactgtggccagcacgggaggcacagacaccaaa | |
| | agcttcatcttggtgagaaaagcagacatggctgatatcccaggccacgtcttcacaagaggaatgatcatagctgtttt | |
| | gatcttggtggcagtagtgtgcctagtgactgtgtgtgtcatttatagagttgacttggttctattttatagacatttaacgag | |
| | aagagatgaaacattaacagatggaaaaacatatgatgcttttgtgtcttacctaaaagaatgccgacctgaaaatggag | |
| | aggagcacacctttgctgtggagattttgcccagggtgttggagaaacattttgggtataagttatgcatatttgaaaggg | |
| | atgtagtgcctggaggagctgttgttgatgaaatccactcactgatagagaaaagccgaagactaatcattgtcctaagt | |
| | aaaagttatatgtctaatgaggtcaggtatgaacttgaaagtggactccatgaagcattggtggaaagaaaaattaaaat | |
| | aatcttaattgaatttacacctgttactgacttcacattcttgccccaatcactaaagcttttgaaatctcacagagttctgaag | |
| | tggaaggccgataaatctctttcttataactcaaggttctggaagaaccttctttacttaatgcctgcaaaaacagtcaagc | |
| | caggtagagacgaaccggaagtcttgcctgttctttccgagtcttgataa | |
| CAR 20A10N | atggccttaccagtgaccgccttgctcctgccgctggccttgctgctccacgccgccaggccgcaggtgcagctggtt | 186 |
| CD8-CD8-41BB | gaatctggcggcggacttgtgaagcctggcggatctctgagactgagctgtgccgccagcggcttcacctttagcacat | |
| 1XX | acgccatgagctggatcagacaggcccctggcaaaggcctggaatgggtggcgtctattggcagagccggcagcac | |
| 6XIL12NFAT | ctactacagcgactctgtgaagggcagattcaccatcagccgggacaacgccaagaacagcctgtacctgcagatga | |
| mCMV | actccctgagagccgaggacaccgccgtgtactattgtgccagaggacccatctacaacgactacgacgagttcgcct | |
| IL18Ralpha | attggggccagggcacactggtcacagtttctagcggcggtggcggaagcggaggcggtggctccggtggcggag | |
| | gcagcgaaattgtgctgacacagagccccgccacactgtcactttctccaggcgaaagagccacactgagctgcaag | |
| | agcagccagagcgtgctgtactccagcaaccagaagaactacctggcctggtatcagcagaagcccggccaagctc | |
| | ctcggctgctgatctattgggccagcacaagagagagcggcatccctgccagattttctggcagcggctctggcaccg | |
| | atttcaccctgaccataagcagcctggaacctgaggacttcgccgtgtattactgccaccagtacctgagcagcctgac | |
| | ctttggcggaggcaccaaggtggaaatcaagcggacaacaacccctgcccccagacctcctaccccagcccctaca | |
| | attgccagccagcctctgagcctgaggcccgaggcttgtagacctgctgctggcggagccgtgcacaccagaggact | |
| | ggatttcgcctgcgacatctacatctgggcgcccttggccgggacttgtggggtccttctcctgtcactggttatcaccct | |
| | ttactgcaaacggggcagaaagaaactcctgtatatattcaaacaaccatttatgagaccagtacaaactactcaagagg | |
| | aagatggctgtagctgccgatttccagaagaagaagaaggaggatgtgaactgagagtgaagttcagcaggagcgc | |
| | agacgcccccgcgtacaagcagggccagaaccagctctataacgagctcaatctaggacgaagagaggagtacgat | |
| | gttttggacaagagacgtggccgggaccctgagatggggggaaagccgagaaggaagaaccctcaggaaggcctg | |
| | ttcaatgaactgcagaaagataagatggcggaggccttcagtgagattgggatgaaaggcgagcgccggaggggca | |
| | aggggcacgatggccttttccagggtctcagtacagccaccaaggacaccttcgacgcccttcacatgcaggccctgc | |
| | cccctcgctgataagtttaaactgccagaacatttctctggcctaactggccggtaccggaggaaaaactgtttcataca | |
| | gaaggcgtggaggaaaaactgtttcatacagaaggcgtggaggaaaaactgtttcatacagaaggcgtggaggaaaa | |
| | actgtttcatacagaaggcgtggaggaaaaactgtttcatacagaaggcgtggaggaaaaactgtttcatacagaaggc | |
| | gtactagttaggcgtgtacggtgggaggcctatataagcagagctcgtttagtgaaccgtcagatcgcctggagacgc | |
| | catccacgctgttttgacctccatagaagacaccgggaccgatccagcctcgagagacccaatgctagccaccatgaa | |
| | ttgtagagaattacccttgaccctttgggtgcttatatctgtaagcactgcagaatcttgtacttcacgtccccacattactgt | |
| | ggttgaaggggaacctttctatctgaaacattgctcgtgttcacttgcacatgagattgaaacaaccaccaaaagctggta | |
| | caaaagcagtggatcacaggaacatgtggagctgaacccaaggagttccagcagaattgctttgcatgattgtgttttgg | |
| | agttttggccagttgagttgaatgacacaggatcttactttttccaaatgaaaaattatactcagaaatggaaattaaatgtc | |
| | atcagaagaaataaacacagctgtttcactgaaagacaagtaactagtaaaattgtggaagttaaaaaattttttcagataa | |
| | cctgtgaaaacagttactatcaaacactggtcaacagcacatcattgtataagaactgtaaaaagctactactggagaac | |
| | aataaaaacccaacgataaagaagaacgccgagtttgaagatcaggggtattactcctgcgtgcatttccttcatcataat | |
| | ggaaaactatttaatatcaccaaaaccttcaatataacaatagtggaagatcgcagtaatatagttccggttcttcttggac | |
| | caaagcttaaccatgttgcagtggaattaggaaaaaacgtaaggctcaactgctctgctttgctgaatgaagaggatgta | |
| | atttattggatgttcggggaagaaaatggatcggatcctaatatacatgaagagaaagaaatgagaattatgactccaga | |
| | aggcaaatggcatgcttcaaaagtattgagaattgaaaatattggtgaaagcaatctaaatgttttatataattgcactgtg | |
| | gccagcacgggaggcacagacaccaaaagcttcatcttggtgagaaaagcagacatggctgatatcccaggccacg | |
| | tcttcacaagaggaatgatcatagctgttttgatcttggtggcagtagtgtgcctagtgactgtgtgtgtcatttatagagtt | |
| | gacttggttctattttatagacatttaacgagaagagatgaaacattaacagatggaaaaacatatgatgcttttgtgtctta | |
| | cctaaaagaatgccgacctgaaaatggagaggagcacacctttgctgtggagattttgcccagggtgttggagaaaca | |
| | ttttgggtataagttatgcatatttgaaagggatgtagtgcctggaggagctgttgttgatgaaatccactcactgatagag | |
| | aaaagccgaagactaatcattgtcctaagtaaaagttatatgtctaatgaggtcaggtatgaacttgaaagtggactccat | |
| | gaagcattggtggaaagaaaaattaaaataatcttaattgaatttacacctgttactgacttcacattcttgccccaatcact | |
| | aaagcttttgaaatctcacagagttctgaagtggaaggccgataaatctctttcttataactcaaggttctggaagaacctt | |
| | ctttacttaatgcctgcaaaaacagtcaagccaggtagagacgaaccggaagtcttgcctgttctttccgagtcttgataa | |
| CAR 20A10N | atggccttaccagtgaccgccttgctcctgccgctggccttgctgctccacgccgccaggccgcaggtgcagctggtt | 187 |
| CD8-CD8-41BB | gaatctggcggcggacttgtgaagcctggcggatctctgagactgagctgtgccgccagcggcttcacctttagcacat | |
| 1XX | acgccatgagctggatcagacaggcccctggcaaaggcctggaatgggtggcgtctattggcagagccggcagcac | |
| 6XFOXP3NFAT | ctactacagcgactctgtgaagggcagattcaccatcagccgggacaacgccaagaacagcctgtacctgcagatga | |
| mCMV | actccctgagagccgaggacaccgccgtgtactattgtgccagaggacccatctacaacgactacgacgagttcgcct | |
| IL18Ralpha-T2A- | attggggccagggcacactggtcacagtttctagcggcggtggcggaagcggaggcggtggctccggtggcggag | |
| IL18Rb | gcagcgaaattgtgctgacacagagccccgccacactgtcactttctccaggcgaaagagccacactgagctgcaag | |
| | agcagccagagcgtgctgtactccagcaaccagaagaactacctggcctggtatcagcagaagcccggccaagctc | |
| | ctcggctgctgatctattgggccagcacaagagagagcggcatccctgccagattttctggcagcggctctggcaccg | |
| | atttcaccctgaccataagcagcctggaacctgaggacttcgccgtgtattactgccaccagtacctgagcagcctgac | |
| | ctttggcggaggcaccaaggtggaaatcaagcggacaacaacccctgcccccagacctcctaccccagcccctaca | |
| | attgccagccagcctctgagcctgaggcccgaggcttgtagacctgctgctggcggagccgtgcacaccagaggact | |
| | ggatttcgcctgcgacatctacatctgggcgcccttggccgggacttgtggggtccttctcctgtcactggttatcaccct | |
| | ttactgcaaacggggcagaaagaaactcctgtatatattcaaacaaccatttatgagaccagtacaaactactcaagagg | |
| | aagatggctgtagctgccgatttccagaagaagaagaaggaggatgtgaactgagagtgaagttcagcaggagcgc | |
| | agacgcccccgcgtacaagcagggccagaaccagctctataacgagctcaatctaggacgaagagaggagtacgat | |
| | gttttggacaagagacgtggccgggaccctgagatggggggaaagccgagaaggaagaaccctcaggaaggcctg | |
| | ttcaatgaactgcagaaagataagatggcggaggccttcagtgagattgggatgaaaggcgagcgccggaggggca | |
| | aggggcacgatggccttttccagggtctcagtacagccaccaaggacaccttcgacgcccttcacatgcaggccctgc | |
| | cccctcgctgataagtttaaactgccagaacatttctctggcctaactggccggtaccggcttcattttttccatttactgca | |
| | gaggcttcattttttccatttactgcagaggcttcattttttccatttactgcagaggcttcattttttccatttactgcagaggct | |
| | tcattttttccatttactgcagaggcttcattttttccatttactgcagaactagttaggcgtgtacggtgggaggcctatataa | |
| | gcagagctcgtttagtgaaccgtcagatcgcctggagacgccatccacgctgttttgacctccatagaagacaccggg | |
| | accgatccagcctcgagagacccaatgctagccaccatgaattgtagagaattacccttgaccctttgggtgcttatatc | |
| | tgtaagcactgcagaatcttgtacttcacgtccccacattactgtggttgaaggggaacctttctatctgaaacattgctcgt | |
| | gttcacttgcacatgagattgaaacaaccaccaaaagctggtacaaaagcagtggatcacaggaacatgtggagctga | |
| | acccaaggagttccagcagaattgctttgcatgattgtgttttggagttttggccagttgagttgaatgacacaggatctta | |
| | ctttttccaaatgaaaaattatactcagaaatggaaattaaatgtcatcagaagaaataaacacagctgtttcactgaaaga | |
| | caagtaactagtaaaattgtggaagttaaaaaattttttcagataacctgtgaaaacagttactatcaaacactggtcaaca | |
| | gcacatcattgtataagaactgtaaaaagctactactggagaacaataaaaacccaacgataaagaagaacgccgagt | |
| | ttgaagatcaggggtattactcctgcgtgcatttccttcatcataatggaaaactatttaatatcaccaaaaccttcaatataa | |
| | caatagtggaagatcgcagtaatatagttccggttcttcttggaccaaagcttaaccatgttgcagtggaattaggaaaaa | |
| | acgtaaggctcaactgctctgctttgctgaatgaagaggatgtaatttattggatgttcggggaagaaaatggatcggat | |
| | cctaatatacatgaagagaaagaaatgagaattatgactccagaaggcaaatggcatgcttcaaaagtattgagaattga | |
| | aaatattggtgaaagcaatctaaatgttttatataattgcactgtggccagcacgggaggcacagacaccaaaagcttca | |
| | tcttggtgagaaaagcagacatggctgatatcccaggccacgtcttcacaagaggaatgatcatagctgttttgatcttgg | |
| | tggcagtagtgtgcctagtgactgtgtgtgtcatttatagagttgacttggttctattttatagacatttaacgagaagagatg | |
| | aaacattaacagatggaaaaacatatgatgcttttgtgtcttacctaaaagaatgccgacctgaaaatggagaggagcac | |
| | acctttgctgtggagattttgcccagggtgttggagaaacattttgggtataagttatgcatatttgaaagggatgtagtgc | |
| | ctggaggagctgttgttgatgaaatccactcactgatagagaaaagccgaagactaatcattgtcctaagtaaaagttata | |
| | tgtctaatgaggtcaggtatgaacttgaaagtggactccatgaagcattggtggaaagaaaaattaaaataatcttaattg | |
| | aatttacacctgttactgacttcacattcttgccccaatcactaaagcttttgaaatctcacagagttctgaagtggaaggcc | |
| | gataaatctctttcttataactcaaggttctggaagaaccttctttacttaatgcctgcaaaaacagtcaagccaggtagag | |
| | acgaaccggaagtcttgcctgttctttccgagtctggaagcggtgagggaagaggtagcctgctcacgtgcggtgatg | |
| | tggaggagaaccctgggcctatgctctgtttgggctggatatttctttggcttgttgcaggagagcgaattaaaggattta | |
| | atatttcaggttgttccacaaaaaaactcctttggacatattctacaaggagtgaagaggaatttgtcttattttgtgatttacc | |
| | agagccacagaaatcacatttctgccacagaaatcgactctcaccaaaacaagtccctgagcacctgcccttcatgggt | |
| | agtaacgacctatctgatgtccaatggtaccaacaaccttcgaatggagatccattagaggacattaggaaaagctatcc | |
| | tcacatcattcaggacaaatgtacccttcactttttgaccccaggggtgaataattctgggtcatatatttgtagacccaag | |
| | atgattaagagcccctatgatgtagcctgttgtgtcaagatgattttagaagttaagccccagacaaatgcatcctgtgagt | |
| | attccgcatcacataagcaagacctacttcttgggagcactggctctatttcttgccccagtctcagctgccaaagtgatg | |
| | cacaaagtccagcggtaacctggtacaagaatggaaaactcctctctgtggaaaggagcaaccgaatcgtagtggatg | |
| | aagtttatgactatcaccagggcacatatgtatgtgattacactcagtcggatactgtgagttcgtggacagtcagagctg | |
| | ttgttcaagtgagaaccattgtgggagacactaaactcaaaccagatattctggatcctgtcgaggacacactggaagta | |
| | gaacttggaaagcctttaactattagctgcaaagcacgatttggctttgaaagggtctttaaccctgtcataaaatggtaca | |
| | tcaaagattctgacctagagtgggaagtctcagtacctgaggcgaaaagtattaaatccactttaaaggatgaaatcattg | |
| | agcgtaatatcatcttggaaaaagtcactcagcgtgatcttcgcaggaagtttgtttgctttgtccagaactccattggaaa | |
| | cacaacccagtccgtccaactgaaagaaaagagaggagtggtgctcctgtacatcctgcttggcaccatcgggaccct | |
| | ggtggccgtgctggcggcgagtgccctcctctacaggcactggattgaaatagtgctgctgtaccggacctaccagag | |
| | caaggatcagacgcttggggataaaaaggattttgatgctttcgtatcctatgcaaaatggagctcttttccaagtgaggc | |
| | cacttcatctctgagtgaagaacacttggccctgagcctatttcctgatgttttagaaaacaaatatggatatagcctgtgtt | |
| | tgcttgaaagagatgtggctccaggaggagtgtatgcagaagacattgtgagcattattaagagaagcagaagaggaa | |
| | tatttatcttgagccccaactatgtcaatggacccagtatctttgaactacaagcagcagtgaatcttgccttggatgatca | |
| | aacactgaaactcattttaattaagttctgttacttccaagagccagagtctctacctcatctcgtgaaaaaagctctcagg | |
| | gttttgcccacagttacttggagaggcttaaaatcagttcctcccaattctaggttctgggccaaaatgcgctaccacatg | |
| | cctgtgaaaaactctcagggattcacgtggaaccagctcagaattacctctaggatttttcagtggaaaggactcagtag | |
| | aacagaaaccactgggaggagctcccagcctaaggaatggtgataa | |
| CAR 20A10N | atggccttaccagtgaccgccttgctcctgccgctggccttgctgctccacgccgccaggccgcaggtgcagctggtt | 188 |
| CD8-CD8-41BB | gaatctggcggcggacttgtgaagcctggcggatctctgagactgagctgtgccgccagcggcttcacctttagcacat | |
| 1XX | acgccatgagctggatcagacaggcccctggcaaaggcctggaatgggtggcgtctattggcagagccggcagcac | |
| 3XFOXP3NFAT | ctactacagcgactctgtgaagggcagattcaccatcagccgggacaacgccaagaacagcctgtacctgcagatga | |
| mCMV | actccctgagagccgaggacaccgccgtgtactattgtgccagaggacccatctacaacgactacgacgagttcgcct | |
| IL18Ralpha-T2A- | attggggccagggcacactggtcacagtttctagcggcggtggcggaagcggaggcggtggctccggtggcggag | |
| IL18Rb | gcagcgaaattgtgctgacacagagccccgccacactgtcactttctccaggcgaaagagccacactgagctgcaag | |
| | agcagccagagcgtgctgtactccagcaaccagaagaactacctggcctggtatcagcagaagcccggccaagctc | |
| | ctcggctgctgatctattgggccagcacaagagagagcggcatccctgccagattttctggcagcggctctggcaccg | |
| | atttcaccctgaccataagcagcctggaacctgaggacttcgccgtgtattactgccaccagtacctgagcagcctgac | |
| | ctttggcggaggcaccaaggtggaaatcaagcggacaacaacccctgcccccagacctcctaccccagcccctaca | |
| | attgccagccagcctctgagcctgaggcccgaggcttgtagacctgctgctggcggagccgtgcacaccagaggact | |
| | ggatttcgcctgcgacatctacatctgggcgcccttggccgggacttgtggggtccttctcctgtcactggttatcaccct | |
| | ttactgcaaacggggcagaaagaaactcctgtatatattcaaacaaccatttatgagaccagtacaaactactcaagagg | |
| | aagatggctgtagctgccgatttccagaagaagaagaaggaggatgtgaactgagagtgaagttcagcaggagcgc | |
| | agacgcccccgcgtacaagcagggccagaaccagctctataacgagctcaatctaggacgaagagaggagtacgat | |
| | gttttggacaagagacgtggccgggaccctgagatggggggaaagccgagaaggaagaaccctcaggaaggcctg | |
| | ttcaatgaactgcagaaagataagatggcggaggccttcagtgagattgggatgaaaggcgagcgccggaggggca | |
| | aggggcacgatggccttttccagggtctcagtacagccaccaaggacaccttcgacgcccttcacatgcaggccctgc | |
| | cccctcgctgataagtttaaactgccagaacatttctctggcctaactggccggtaccggcttcattttttccatttactgca | |
| | gaggcttcattttttccatttactgcagaggcttcattttttccatttactgcagaactagttaggcgtgtacggtgggaggc | |
| | ctatataagcagagctcgtttagtgaaccgtcagatcgcctggagacgccatccacgctgttttgacctccatagaagac | |
| | accgggaccgatccagcctcgagagacccaatgctagccaccatgaattgtagagaattacccttgaccctttgggtg | |
| | cttatatctgtaagcactgcagaatcttgtacttcacgtccccacattactgtggttgaaggggaacctttctatctgaaaca | |
| | ttgctcgtgttcacttgcacatgagattgaaacaaccaccaaaagctggtacaaaagcagtggatcacaggaacatgtg | |
| | gagctgaacccaaggagttccagcagaattgctttgcatgattgtgttttggagttttggccagttgagttgaatgacaca | |
| | ggatcttactttttccaaatgaaaaattatactcagaaatggaaattaaatgtcatcagaagaaataaacacagctgtttcac | |
| | tgaaagacaagtaactagtaaaattgtggaagttaaaaaattttttcagataacctgtgaaaacagttactatcaaacactg | |
| | gtcaacagcacatcattgtataagaactgtaaaaagctactactggagaacaataaaaacccaacgataaagaagaac | |
| | gccgagtttgaagatcaggggtattactcctgcgtgcatttccttcatcataatggaaaactatttaatatcaccaaaacctt | |
| | caatataacaatagtggaagatcgcagtaatatagttccggttcttcttggaccaaagcttaaccatgttgcagtggaatta | |
| | ggaaaaaacgtaaggctcaactgctctgctttgctgaatgaagaggatgtaatttattggatgttcggggaagaaaatgg | |
| | atcggatcctaatatacatgaagagaaagaaatgagaattatgactccagaaggcaaatggcatgcttcaaaagtattga | |
| | gaattgaaaatattggtgaaagcaatctaaatgttttatataattgcactgtggccagcacgggaggcacagacaccaaa | |
| | agcttcatcttggtgagaaaagcagacatggctgatatcccaggccacgtcttcacaagaggaatgatcatagctgtttt | |
| | gatcttggtggcagtagtgtgcctagtgactgtgtgtgtcatttatagagttgacttggttctattttatagacatttaacgag | |
| | aagagatgaaacattaacagatggaaaaacatatgatgcttttgtgtcttacctaaaagaatgccgacctgaaaatggag | |
| | aggagcacacctttgctgtggagattttgcccagggtgttggagaaacattttgggtataagttatgcatatttgaaaggg | |
| | atgtagtgcctggaggagctgttgttgatgaaatccactcactgatagagaaaagccgaagactaatcattgtcctaagt | |
| | aaaagttatatgtctaatgaggtcaggtatgaacttgaaagtggactccatgaagcattggtggaaagaaaaattaaaat | |
| | aatcttaattgaatttacacctgttactgacttcacattcttgccccaatcactaaagcttttgaaatctcacagagttctgaag | |
| | tggaaggccgataaatctctttcttataactcaaggttctggaagaaccttctttacttaatgcctgcaaaaacagtcaagc | |
| | caggtagagacgaaccggaagtcttgcctgttctttccgagtctggaagcggtgagggaagaggtagcctgctcacgt | |
| | gcggtgatgtggaggagaaccctgggcctatgctctgtttgggctggatatttctttggcttgttgcaggagagcgaatta | |
| | aaggatttaatatttcaggttgttccacaaaaaaactcctttggacatattctacaaggagtgaagaggaatttgtcttatttt | |
| | gtgatttaccagagccacagaaatcacatttctgccacagaaatcgactctcaccaaaacaagtccctgagcacctgcc | |
| | cttcatgggtagtaacgacctatctgatgtccaatggtaccaacaaccttcgaatggagatccattagaggacattagga | |
| | aaagctatcctcacatcattcaggacaaatgtacccttcactttttgaccccaggggtgaataattctgggtcatatatttgt | |
| | agacccaagatgattaagagcccctatgatgtagcctgttgtgtcaagatgattttagaagttaagccccagacaaatgc | |
| | atcctgtgagtattccgcatcacataagcaagacctacttcttgggagcactggctctatttcttgccccagtctcagctgc | |
| | caaagtgatgcacaaagtccagcggtaacctggtacaagaatggaaaactcctctctgtggaaaggagcaaccgaat | |
| | cgtagtggatgaagtttatgactatcaccagggcacatatgtatgtgattacactcagtcggatactgtgagttcgtggac | |
| | agtcagagctgttgttcaagtgagaaccattgtgggagacactaaactcaaaccagatattctggatcctgtcgaggaca | |
| | cactggaagtagaacttggaaagcctttaactattagctgcaaagcacgatttggctttgaaagggtctttaaccctgtcat | |
| | aaaatggtacatcaaagattctgacctagagtgggaagtctcagtacctgaggcgaaaagtattaaatccactttaaagg | |
| | atgaaatcattgagcgtaatatcatcttggaaaaagtcactcagcgtgatcttcgcaggaagtttgtttgctttgtccagaa | |
| | ctccattggaaacacaacccagtccgtccaactgaaagaaaagagaggagtggtgctcctgtacatcctgcttggcac | |
| | catcgggaccctggtggccgtgctggcggcgagtgccctcctctacaggcactggattgaaatagtgctgctgtaccg | |
| | gacctaccagagcaaggatcagacgcttggggataaaaaggattttgatgctttcgtatcctatgcaaaatggagctcttt | |
| | tccaagtgaggccacttcatctctgagtgaagaacacttggccctgagcctatttcctgatgttttagaaaacaaatatgg | |
| | atatagcctgtgtttgcttgaaagagatgtggctccaggaggagtgtatgcagaagacattgtgagcattattaagagaa | |
| | gcagaagaggaatatttatcttgagccccaactatgtcaatggacccagtatctttgaactacaagcagcagtgaatcttg | |
| | ccttggatgatcaaacactgaaactcattttaattaagttctgttacttccaagagccagagtctctacctcatctcgtgaaa | |
| | aaagctctcagggttttgcccacagttacttggagaggcttaaaatcagttcctcccaattctaggttctgggccaaaatg | |
| | cgctaccacatgcctgtgaaaaactctcagggattcacgtggaaccagctcagaattacctctaggatttttcagtggaa | |
| | aggactcagtagaacagaaaccactgggaggagctcccagcctaaggaatggtgataa | |
| CAR 20A10N | atggccttaccagtgaccgccttgctcctgccgctggccttgctgctccacgccgccaggccgcaggtgcagctggtt | 189 |
| CD8-CD8-41BB | gaatctggcggcggacttgtgaagcctggcggatctctgagactgagctgtgccgccagcggcttcacctttagcacat | |
| 1XX | acgccatgagctggatcagacaggcccctggcaaaggcctggaatgggtggcgtctattggcagagccggcagcac | |
| 6XIL12NFAT | ctactacagcgactctgtgaagggcagattcaccatcagccgggacaacgccaagaacagcctgtacctgcagatga | |
| mCMV | actccctgagagccgaggacaccgccgtgtactattgtgccagaggacccatctacaacgactacgacgagttcgcct | |
| IL18Ralpha-T2A- | attggggccagggcacactggtcacagtttctagcggcggtggcggaagcggaggcggtggctccggtggcggag | |
| IL18Rb | gcagcgaaattgtgctgacacagagccccgccacactgtcactttctccaggcgaaagagccacactgagctgcaag | |
| | agcagccagagcgtgctgtactccagcaaccagaagaactacctggcctggtatcagcagaagcccggccaagctc | |
| | ctcggctgctgatctattgggccagcacaagagagagcggcatccctgccagattttctggcagcggctctggcaccg | |
| | atttcaccctgaccataagcagcctggaacctgaggacttcgccgtgtattactgccaccagtacctgagcagc ctgac | |
| | ctttggcggaggcaccaaggtggaaatcaagcggacaacaacccctgcccccagacctcctaccccagcccctaca | |
| | attgccagccagcctctgagcctgaggcccgaggcttgtagacctgctgctggcggagccgtgcacaccagaggact | |
| | ggatttcgcctgcgacatctacatctgggcgcccttggccgggacttgtggggtccttctcctgtcactggttatcaccct | |
| | ttactgcaaacggggcagaaagaaactcctgtatatattcaaacaaccatttatgagaccagtacaaactactcaagagg | |
| | aagatggctgtagctgccgatttccagaagaagaagaaggaggatgtgaactgagagtgaagttcagcaggagcgc | |
| | agacgcccccgcgtacaagcagggccagaaccagctctataacgagctcaatctaggacgaagagaggagtacgat | |
| | gttttggacaagagacgtggccgggaccctgagatggggggaaagccgagaaggaagaaccctcaggaaggcctg | |
| | ttcaatgaactgcagaaagataagatggcggaggccttcagtgagattgggatgaaaggcgagcgccggaggggca | |
| | aggggcacgatggccttttccagggtctcagtacagccaccaaggacaccttcgacgcccttcacatgcaggccctgc | |
| | cccctcgctgataagtttaaactgccagaacatttctctggcctaactggccggtaccggaggaaaaactgtttcataca | |
| | gaaggcgtggaggaaaaactgtttcatacagaaggcgtggaggaaaaactgtttcatacagaaggcgtggaggaaaa | |
| | actgtttcatacagaaggcgtggaggaaaaactgtttcatacagaaggcgtggaggaaaaactgtttcatacagaaggc | |
| | gtactagttaggcgtgtacggtgggaggcctatataagcagagctcgtttagtgaaccgtcagatcgcctggagacgc | |
| | catccacgctgttttgacctccatagaagacaccgggaccgatccagcctcgagagacccaatgctagccaccatga | |
| | attgtagagaattacccttgaccctttgggtgcttatatctgtaagcactgcagaatcttgtacttcacgtccccacattactg | |
| | tggttgaaggggaacctttctatctgaaacattgctcgtgttcacttgcacatgagattgaaacaaccaccaaaagctggt | |
| | acaaaagcagtggatcacaggaacatgtggagctgaacccaaggagttccagcagaattgctttgcatgattgtgttttg | |
| | gagttttggccagttgagttgaatgacacaggatcttactttttccaaatgaaaaattatactcagaaatggaaattaaatgt | |
| | catcagaagaaataaacacagctgtttcactgaaagacaagtaactagtaaaattgtggaagttaaaaaattttttcagata | |
| | acctgtgaaaacagttactatcaaacactggtcaacagcacatcattgtataagaactgtaaaaagctactactggagaa | |
| | caataaaaacccaacgataaagaagaacgccgagtttgaagatcaggggtattactcctgcgtgcatttccttcatcata | |
| | atggaaaactatttaatatcaccaaaaccttcaatataacaatagtggaagatcgcagtaatatagttccggttcttcttgga | |
| | ccaaagcttaaccatgttgcagtggaattaggaaaaaacgtaaggctcaactgctctgctttgctgaatgaagaggatgt | |
| | aatttattggatgttcggggaagaaaatggatcggatcctaatatacatgaagagaaagaaatgagaattatgactccag | |
| | aaggcaaatggcatgcttcaaaagtattgagaattgaaaatattggtgaaagcaatctaaatgttttatataattgcactgtg | |
| | gccagcacgggaggcacagacaccaaaagcttcatcttggtgagaaaagcagacatggctgatatcccaggccacg | |
| | tcttcacaagaggaatgatcatagctgttttgatcttggtggcagtagtgtgcctagtgactgtgtgtgtcatttatagagtt | |
| | gacttggttctattttatagacatttaacgagaagagatgaaacattaacagatggaaaaacatatgatgcttttgtgtctta | |
| | cctaaaagaatgccgacctgaaaatggagaggagcacacctttgctgtggagattttgcccagggtgttggagaaaca | |
| | ttttgggtataagttatgcatatttgaaagggatgtagtgcctggaggagctgttgttgatgaaatccactcactgatagag | |
| | aaaagccgaagactaatcattgtcctaagtaaaagttatatgtctaatgaggtcaggtatgaacttgaaagtggactccat | |
| | gaagcattggtggaaagaaaaattaaaataatcttaattgaatttacacctgttactgacttcacattcttgccccaatcact | |
| | aaagcttttgaaatctcacagagttctgaagtggaaggccgataaatctctttcttataactcaaggttctggaagaacctt | |
| | ctttacttaatgcctgcaaaaacagtcaagccaggtagagacgaaccggaagtcttgcctgttctttccgagtctggaag | |
| | cggtgagggaagaggtagcctgctcacgtgcggtgatgtggaggagaaccctgggcctatgctctgtttgggctggat | |
| | atttctttggcttgttgcaggagagcgaattaaaggatttaatatttcaggttgttccacaaaaaaactcctttggacatattct | |
| | acaaggagtgaagaggaatttgtcttattttgtgatttaccagagccacagaaatcacatttctgccacagaaatcgactct | |
| | caccaaaacaagtccctgagcacctgcccttcatgggtagtaacgacctatctgatgtccaatggtaccaacaaccttcg | |
| | aatggagatccattagaggacattaggaaaagctatcctcacatcattcaggacaaatgtacccttcactttttgacccca | |
| | ggggtgaataattctgggtcatatatttgtagacccaagatgattaagagcccctatgatgtagcctgttgtgtcaagatg | |
| | attttagaagttaagccccagacaaatgcatcctgtgagtattccgcatcacataagcaagacctacttcttgggagcact | |
| | ggctctatttcttgccccagtctcagctgccaaagtgatgcacaaagtccagcggtaacctggtacaagaatggaaaac | |
| | tcctctctgtggaaaggagcaaccgaatcgtagtggatgaagtttatgactatcaccagggcacatatgtatgtgattaca | |
| | ctcagtcggatactgtgagttcgtggacagtcagagctgttgttcaagtgagaaccattgtgggagacactaaactcaaa | |
| | ccagatattctggatcctgtcgaggacacactggaagtagaacttggaaagcctttaactattagctgcaaagcacgattt | |
| | ggctttgaaagggtctttaaccctgtcataaaatggtacatcaaagattctgacctagagtgggaagtctcagtacctgag | |
| | gcgaaaagtattaaatccactttaaaggatgaaatcattgagcgtaatatcatcttggaaaaagtcactcagcgtgatcttc | |
| | gcaggaagtttgtttgctttgtccagaactccattggaaacacaacccagtccgtccaactgaaagaaaagagaggagt | |
| | ggtgctcctgtacatcctgcttggcaccatcgggaccctggtggccgtgctggcggcgagtgccctcctctacaggca | |
| | ctggattgaaatagtgctgctgtaccggacctaccagagcaaggatcagacgcttggggataaaaaggattttgatgctt | |
| | tcgtatcctatgcaaaatggagctcttttccaagtgaggccacttcatctctgagtgaagaacacttggccctgagcctatt | |
| | tcctgatgttttagaaaacaaatatggatatagcctgtgtttgcttgaaagagatgtggctccaggaggagtgtatgcaga | |
| | agacattgtgagcattattaagagaagcagaagaggaatatttatcttgagccccaactatgtcaatggacccagtatctt | |
| | tgaactacaagcagcagtgaatcttgccttggatgatcaaacactgaaactcattttaattaagttctgttacttccaagagc | |
| | cagagtctctacctcatctcgtgaaaaaagctctcagggttttgcccacagttacttggagaggcttaaaatcagttcctcc | |
| | caattctaggttctgggccaaaatgcgctaccacatgcctgtgaaaaactctcagggattcacgtggaaccagctcaga | |
| | attacctctaggatttttcagtggaaaggactcagtagaacagaaaccactgggaggagctcccagcctaaggaatggt | |
| | gataa | |
| CAR 20A10N | atggccttaccagtgaccgccttgctcctgccgctggccttgctgctccacgccgccaggccgcaggtgcagctggtt | 190 |
| CD8-CD8-41BB | gaatctggcggcggacttgtgaagcctggcggatctctgagactgagctgtgccgccagcggcttcacctttagcacat | |
| 1XX | acgccatgagctggatcagacaggcccctggcaaaggcctggaatgggtggcgtctattggcagagccggcagcac | |
| 3XIL12NFAT | ctactacagcgactctgtgaagggcagattcaccatcagccgggacaacgccaagaacagcctgtacctgcagatga | |
| mCMV | actccctgagagccgaggacaccgccgtgtactattgtgccagaggacccatctacaacgactacgacgagttcgcct | |
| IL18Ralpha-T2A- | attggggccagggcacactggtcacagtttctagcggcggtggcggaagcggaggcggtggctccggtggcggag | |
| IL18Rb | gcagcgaaattgtgctgacacagagccccgccacactgtcactttctccaggcgaaagagccacactgagctgcaag | |
| | agcagccagagcgtgctgtactccagcaaccagaagaactacctggcctggtatcagcagaagcccggccaagctc | |
| | ctcggctgctgatctattgggccagcacaagagagagcggcatccctgccagattttctggcagcggctctggcaccg | |
| | atttcaccctgaccataagcagcctggaacctgaggacttcgccgtgtattactgccaccagtacctgagcagcctgac | |
| | ctttggcggaggcaccaaggtggaaatcaagcggacaacaacccctgcccccagacctcctaccccagcccctaca | |
| | attgccagccagcctctgagcctgaggcccgaggcttgtagacctgctgctggcggagccgtgcacaccagaggact | |
| | ggatttcgcctgcgacatctacatctgggcgcccttggccgggacttgtggggtccttctcctgtcactggttatcaccct | |
| | ttactgcaaacggggcagaaagaaactcctgtatatattcaaacaaccatttatgagaccagtacaaactactcaagagg | |
| | aagatggctgtagctgccgatttccagaagaagaagaaggaggatgtgaactgagagtgaagttcagcaggagcgc | |
| | agacgcccccgcgtacaagcagggccagaaccagctctataacgagctcaatctaggacgaagagaggagtacgat | |
| | gttttggacaagagacgtggccgggaccctgagatggggggaaagccgagaaggaagaaccctcaggaaggcctg | |
| | ttcaatgaactgcagaaagataagatggggaggccttcagtgagattgggatgaaaggcgagcgccggaggggca | |
| | aggggcacgatggccttttccagggtctcagtacagccaccaaggacaccttcgacgcccttcacatgcaggccctgc | |
| | cccctcgctgataagtttaaactgccagaacatttctctggcctaactggccggtaccggaggaaaaactgtttcataca | |
| | gaaggcgtggaggaaaaactgtttcatacagaaggcgtggaggaaaaactgtttcatacagaaggcgtactagttagg | |
| | cgtgtacggtgggaggcctatataagcagagctcgtttagtgaaccgtcagatcgcctggagacgccatccacgctgtt | |
| | ttgacctccatagaagacaccgggaccgatccagcctcgagagacccaatgctagccaccatgaattgtagagaatt | |
| | acccttgaccctttgggtgcttatatctgtaagcactgcagaatcttgtacttcacgtccccacattactgtggttgaaggg | |
| | gaacctttctatctgaaacattgctcgtgttcacttgcacatgagattgaaacaaccaccaaaagctggtacaaaagcagt | |
| | ggatcacaggaacatgtggagctgaacccaaggagttccagcagaattgctttgcatgattgtgttttggagttttggcc | |
| | agttgagttgaatgacacaggatcttactttttccaaatgaaaaattatactcagaaatggaaattaaatgtcatcagaaga | |
| | aataaacacagctgtttcactgaaagacaagtaactagtaaaattgtggaagttaaaaaattttttcagataacctgtgaaa | |
| | acagttactatcaaacactggtcaacagcacatcattgtataagaactgtaaaaagctactactggagaacaataaaaac | |
| | ccaacgataaagaagaacgccgagtttgaagatcaggggtattactcctgcgtgcatttccttcatcataatggaaaact | |
| | atttaatatcaccaaaaccttcaatataacaatagtggaagatcgcagtaatatagttccggttcttcttggaccaaagctta | |
| | accatgttgcagtggaattaggaaaaaacgtaaggctcaactgctctgctttgctgaatgaagaggatgtaatttattgga | |
| | tgttcggggaagaaaatggatcggatcctaatatacatgaagagaaagaaatgagaattatgactccagaaggcaaat | |
| | ggcatgcttcaaaagtattgagaattgaaaatattggtgaaagcaatctaaatgttttatataattgcactgtggccagcac | |
| | gggaggcacagacaccaaaagcttcatcttggtgagaaaagcagacatggctgatatcccaggccacgtcttcacaa | |
| | gaggaatgatcatagctgttttgatcttggtggcagtagtgtgcctagtgactgtgtgtgtcatttatagagttgacttggttc | |
| | tattttatagacatttaacgagaagagatgaaacattaacagatggaaaaacatatgatgcttttgtgtcttacctaaaagaa | |
| | tgccgacctgaaaatggagaggagcacacctttgctgtggagattttgcccagggtgttggagaaacattttgggtataa | |
| | gttatgcatatttgaaagggatgtagtgcctggaggagctgttgttgatgaaatccactcactgatagagaaaagccgaa | |
| | gactaatcattgtcctaagtaaaagttatatgtctaatgaggtcaggtatgaacttgaaagtggactccatgaagcattggt | |
| | ggaaagaaaaattaaaataatcttaattgaatttacacctgttactgacttcacattcttgccccaatcactaaagcttttgaa | |
| | atctcacagagttctgaagtggaaggccgataaatctctttcttataactcaaggttctggaagaaccttctttacttaatgc | |
| | ctgcaaaaacagtcaagccaggtagagacgaaccggaagtcttgcctgttctttccgagtctggaagcggtgagggaa | |
| | gaggtagcctgctcacgtgcggtgatgtggaggagaaccctgggcctatgctctgtttgggctggatatttctttggcttg | |
| | ttgcaggagagcgaattaaaggatttaatatttcaggttgttccacaaaaaaactcctttggacatattctacaaggagtga | |
| | agaggaatttgtcttattttgtgatttaccagagccacagaaatcacatttctgccacagaaatcgactctcaccaaaacaa | |
| | gtccctgagcacctgcccttcatgggtagtaacgacctatctgatgtccaatggtaccaacaaccttcgaatggagatcc | |
| | attagaggacattaggaaaagctatcctcacatcattcaggacaaatgtacccttcactttttgaccccaggggtgaataa | |
| | ttctgggtcatatatttgtagacccaagatgattaagagcccctatgatgtagcctgttgtgtcaagatgattttagaagtta | |
| | agccccagacaaatgcatcctgtgagtattccgcatcacataagcaagacctacttcttgggagcactggctctatttctt | |
| | gccccagtctcagctgccaaagtgatgcacaaagtccagcggtaacctggtacaagaatggaaaactcctctctgtgg | |
| | aaaggagcaaccgaatcgtagtggatgaagtttatgactatcaccagggcacatatgtatgtgattacactcagtcggat | |
| | actgtgagttcgtggacagtcagagctgttgttcaagtgagaaccattgtgggagacactaaactcaaaccagatattct | |
| | ggatcctgtcgaggacacactggaagtagaacttggaaagcctttaactattagctgcaaagcacgatttggctttgaaa | |
| | gggtctttaaccctgtcataaaatggtacatcaaagattctgacctagagtgggaagtctcagtacctgaggcgaaaagt | |
| | attaaatccactttaaaggatgaaatcattgagcgtaatatcatcttggaaaaagtcactcagcgtgatcttcgcaggaagt | |
| | ttgtttgctttgtccagaactccattggaaacacaacccagtccgtccaactgaaagaaaagagaggagtggtgctcctg | |
| | tacatcctgcttggcaccatcgggaccctggtggccgtgctggcggcgagtgccctcctctacaggcactggattgaa | |
| | atagtgctgctgtaccggacctaccagagcaaggatcagacgcttggggataaaaaggattttgatgctttcgtatcctat | |
| | gcaaaatggagctcttttccaagtgaggccacttcatctctgagtgaagaacacttggccctgagcctatttcctgatgttt | |
| | tagaaaacaaatatggatatagcctgtgtttgcttgaaagagatgtggctccaggaggagtgtatgcagaagacattgtg | |
| | agcattattaagagaagcagaagaggaatatttatcttgagccccaactatgtcaatggacccagtatctttgaactacaa | |
| | gcagcagtgaatcttgccttggatgatcaaacactgaaactcattttaattaagttctgttacttccaagagccagagtctct | |
| | acctcatctcgtgaaaaaagctctcagggttttgcccacagttacttggagaggcttaaaatcagttcctcccaattctagg | |
| | ttctgggccaaaatgcgctaccacatgcctgtgaaaaactctcagggattcacgtggaaccagctcagaattacctctag | |
| | gatttttcagtggaaaggactcagtagaacagaaaccactgggaggagctcccagcctaaggaatggtgataa | |

### EXAMPLES

The following examples are provided to further illustrate some embodiments of the present disclosure, but are not intended to limit the scope of the disclosure; it will be understood by their exemplary nature that other procedures, methodologies, or techniques known to those skilled in the art may alternatively be used. In the figures and figure descriptions, the names of the CAR and non-CAR constructs are often abbreviated. Table 8 below shows name abbreviations and a corresponding description.

TABLE 8
Abbreviated names and corresponding description

| Name Abbreviated | Description |
|---|---|
| MNC2-41BB-3z | A construct comprising a huMNC2 scFv, a 41BB |
| | co-stimulatory domain and a CD3ζ signaling |
| | domain |
| MNC2-41BB-IL18 | A construct comprising a huMNC2 scFv, a 41BB |
| | co-stimulatory domain, a CD3ζ signaling |
| | domain and an inducible IL-18 cytokine |
| MNC2-41BB-3z- | A construct comprising a huMNC2 scFv, a 41BB |
| IL18ra | co-stimulatory domain, a CD3ζ signaling |
| | domain and an inducible IL-18 receptor α |
| MNC2-41BB-IL18ra | A double construct wherein the nucleic acid |
| (2p) | sequences are encoded on two separate plasmids |
| MNC2-41BB-IL18ra | A single construct wherein the nucleic acid |
| (1p) | sequences are encoded on one plasmid |
| MNC2-28-IL18ra | A single construct wherein the nucleic acid |
| 6X (1p) | sequences are encoded on one plasmid driven |
| | by a promoter containing six NFAT response |
| | elements |
| MNC2-28-IL18ra | A single construct wherein the nucleic acid |
| 3X (1p) | sequences are encoded on one plasmid driven |
| | by a promoter containing three NFAT response |
| | elements |
| MNC2-28-IL18ra | A double construct wherein the nucleic acid |
| 6X (2p) | sequences are encoded on two separate plasmids, |
| | where one plasmid is driven by a promoter |
| | containing six NFAT response elements |
| MNC2-41BB-1XX- | A construct comprising a huMNC2 scFv, a 41BB |
| IL18ra | co-stimulatory domain, a CD3ζ with 2 |
| | Tyrosines in ITAM2 and ITAM3 have been |
| | mutated to Phenylalanine, and an inducible |
| | IL-18 receptor α |
| MNC2-41BB-1XX- | A construct comprising a huMNC2 scFv, a 41BB |
| IL18 | co-stimulatory domain, a CD3ζ with 2 |
| | Tyrosines in ITAM2 and ITAM3 have been |
| | mutated to Phenylalanine, and an inducible |
| | IL-18 cytokine |
| MNC2-28-3z | A construct comprising a huMNC2 scFv, a CD28 |
| | co-stimulatory domain and a CD3ζ signaling |
| | domain |
| MNC2-28-3z- | A construct comprising a huMNC2 scFv, a CD28 |
| IL18ra | co-stimulatory domain, a CD3ζ signaling domain |
| | and an inducible IL-18 receptor α |
| MNC2-28-3z- | A construct comprising a huMNC2 scFv, a CD28 |
| IL18ra/b | co-stimulatory domain, a CD3ζ signaling domain |
| | and an inducible IL-18 receptor α and β |
| MNC2-28-1XX | A construct comprising a huMNC2 scFv, a CD28 |
| | co-stimulatory domain and a CD3ζ with 2 |
| | Tyrosines in ITAM2 and ITAM3 have been |
| | mutated to Phenylalanine |
| MNC2-28-1XX- | A construct comprising a huMNC2 scFv, a CD28 |
| IL18ra | co-stimulatory domain, a CD3ζ with 2 |
| | Tyrosines in ITAM2 and ITAM3 have been |
| | mutated to Phenylalanine and aninducible |
| | IL-18 receptor α |
| MNC2-28-1XX-IL | A construct comprising a huMNC2 scFv, a CD28 |
| IL18ra/b | co-stimulatory domain, a CD3ζ with 2 |
| | Tyrosines in ITAM2 and ITAM3 have been |
| | mutated to Phenylalanine and an inducible |
| | IL-18 receptor α and β |
| UT | Untransduced CD3+ T cells |

### Example 1. Expression of IL-18Rα by CAR Constructs

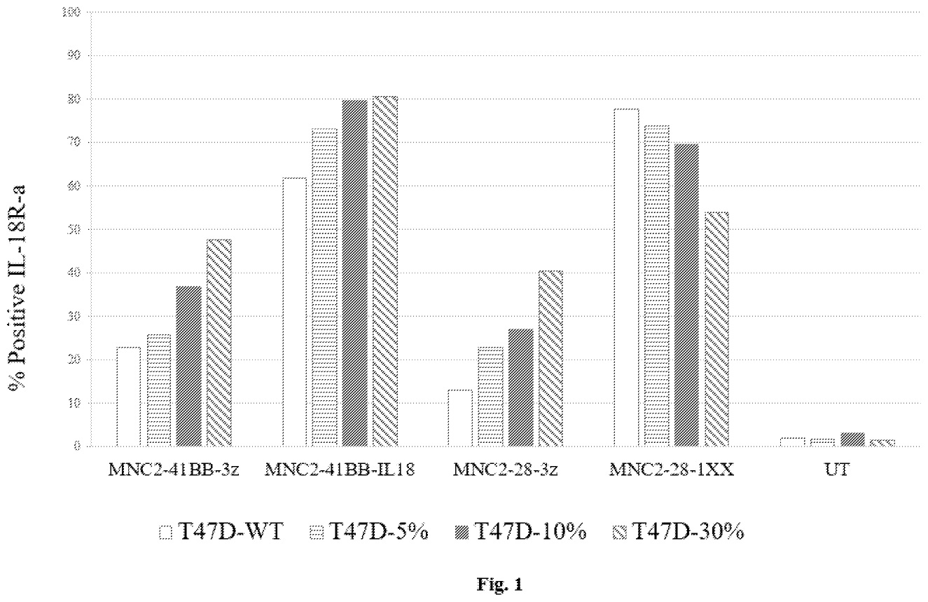

In this example, we used flow cytometry to measure differences in the expression of the IL-18 receptor alpha, referred to here as IL-18Rα. The CAR constructs that were assayed were all directed to the cancer cells via the same anti-MUC1* antibody, huMNC2. MNC2-41BB-3z bears a 41BB co-stimulatory domain and a wild-type CD3ζ signaling domain. MNC2-41BB-IL18 was the same except that it also has an NFAT inducible IL-18 cytokine. In this experiment, the various CAR T cells had been in culture with cancer cells for 72 hours. The panel of cancer cells that were co-cultured with the CAR T cells were breast cancer T47D cells that were wild-type (WT), or a mixed population wherein either 5%, 10% or 30% of the population comprised T47D cells that were engineered to express more MUC1*. The CAR T cells were co-cultured with the cancer cells in RPMI-1640-FBS media at a ratio of 2.5:1. Flow cytometry was performed on a 4-laser Attune NxT (Invitrogen) instrument. IL-18Rα was detected using PE-Cy7 conjugated mouse anti-human IL-18Rα antibody (clone H44; Biolegend #313812) at a concentration of 1.0 ug/mL. We found that the CARs bearing wild-type CD3ζ expressed the IL-18 receptor alpha, also known as IL-18Rα, at much lower levels than the CAR bearing CD3ζ with the 1XX mutations. 1XX mutations are two Tyrosine to phenylalanine mutations in ITAM 1 and two Tyrosine to phenylalanine mutations in ITAM 2. The CAR that inducibly expressed IL-18 also expressed higher levels of IL-18Rα than the control CAR that did not express IL-18. Furthermore, higher expression of the target antigen increased the amount of IL-18Rα expression, presumably because of increased CAR T cell activation. These results strongly argue that signaling through the IL-18Rα confers resistance to CAR T cell exhaustion. These data are shown in FIG. 1.

A graph of flow cytometry as illustrated in FIG. 1 showed that the CARs bearing wild-type CD3-5 expressing the IL-18 receptor alpha, also known as IL-18Rα, at much lower levels than the CAR bearing CD3-ζ with the 1XX mutations and the CAR construct that inducibly expressed IL-18 cytokine. Levels of IL-18Rα expression on four different CAR T cells were measured after co-cultured with cancer cells expressing variable levels of the target antigen (MUC1*). The four CAR T cells were: 1) MNC2-41BB-3z, bearing a 41BB co-stimulatory domain and a wild-type CD32; 2) MNC2-41BB-IL18, was the same as (1) except that the CAR T cell expressing the CAR plus an inducible IL18 cytokine; 3) MNC2-28-3z was a CAR bearing a CD28 co-stimulatory domain; and 4) MNC2-28-1XX was the same as (3) except that its CD3ζ bearing the 4 Tyr to Phe “1XX” mutations. As a control, untransduced T cells from the same donor were assayed for expression of IL-18Rα. The CAR that inducibly expressed IL-18 also expressed higher levels of IL-18Rα than the control CAR that did not express IL-18. It was found that the CARs bearing wild-type CD3ζ expressed the IL-18 receptor alpha, also known as IL-18Rα, at much lower levels than the CAR bearing CD3 with the 1XX mutations. Furthermore, higher expression of the target antigen increased the amount of IL-18Rα expression, presumably because of increased CAR T cell activation. It showed that signaling through the IL-18Rα confers resistance to CAR T cell exhaustion.

### Example 2. Detection of Expression IL-18 Receptor Alpha by Different CAR T Cell Constructs with Repeated Antigen Stimulation

Flow cytometry experiments were carried out to quantify the amount of IL-18 receptor alpha expressed by different CAR T cell constructs in response to cells bearing the target antigen, either before or after several days of repeated antigen stimulation, which is known to induce CAR T cell exhaustion. The target cells were T47D MUC1* positive breast cancer cells that were either wild-type (WT), or mixed with 5%, 10% or 30% of T47D cells that had been engineered to express even more MUC1*. In each case the antibody fragment that directed the CAR to the tumor was humanized MNC2 scFv. Importantly, the cells were either not antigen stimulated before the co-culture or had been repeatedly antigen stimulated before co-culture. The ability of various CAR constructs to continue expressing IL-18Rα after repeated antigen stimulation before being co-cultured with the target cancer cells was tested.

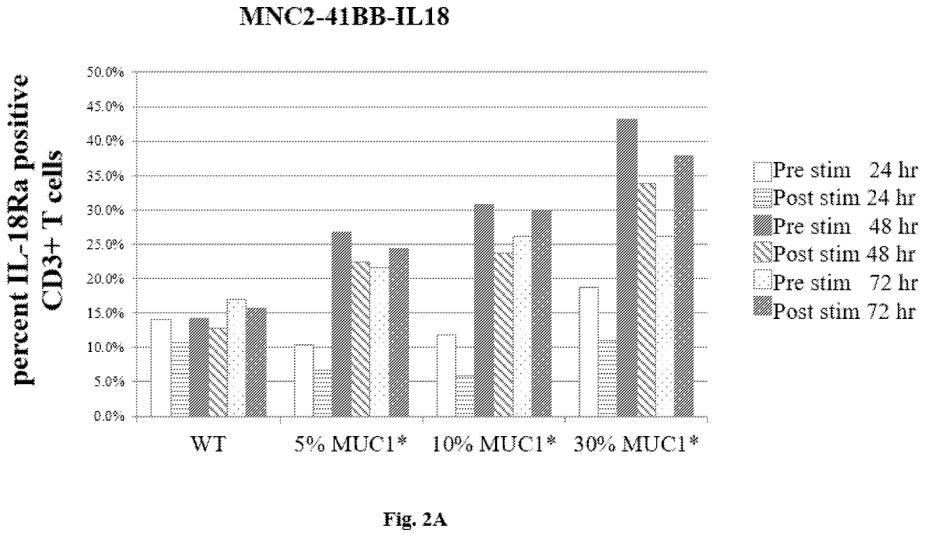

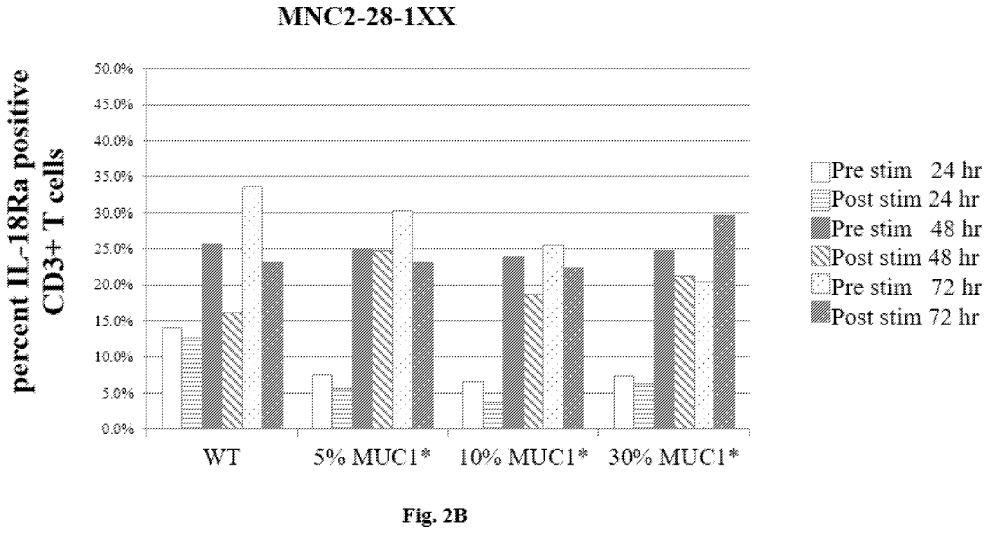

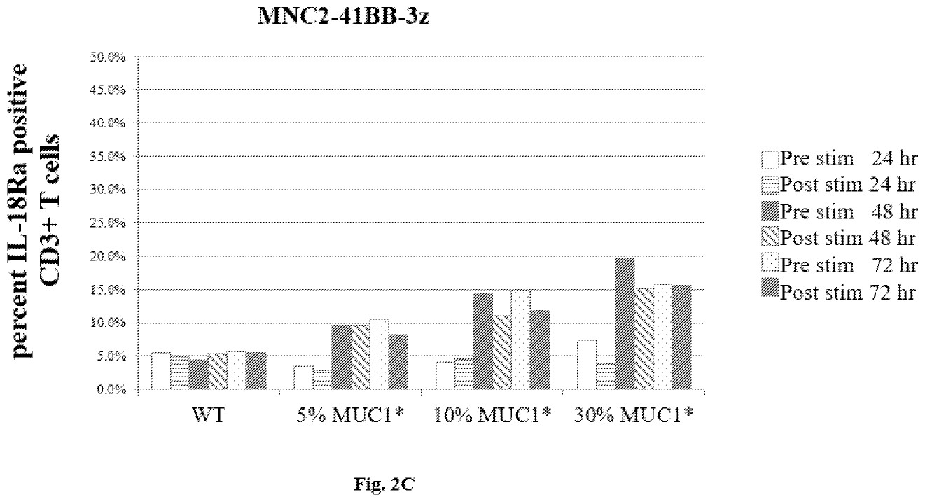

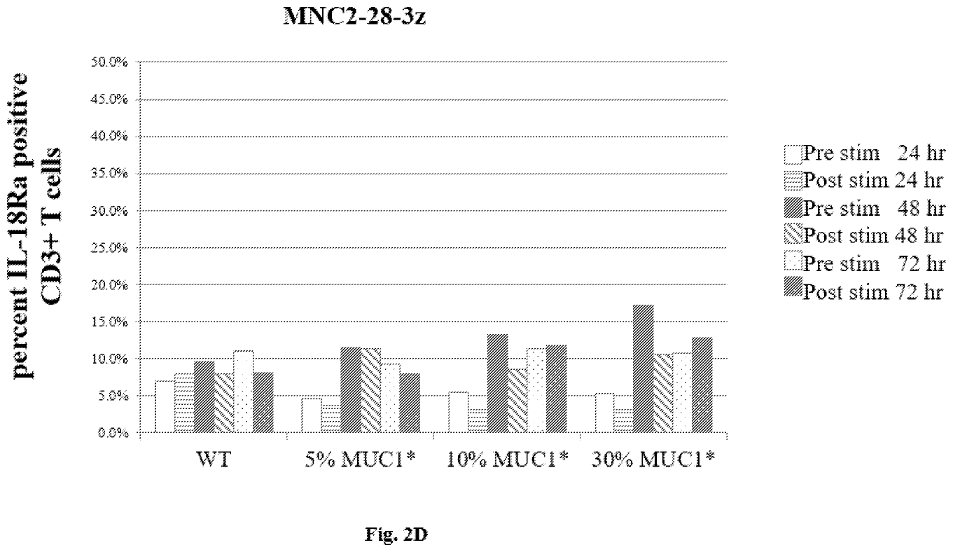

Results from flow cytometry experiments were illustrated in FIGS. 2A-2D. It was found that repeated antigen stimulation, either by stimulating with the target peptide on a bead or by co-culture with cells expressing high levels of antigen, caused a decrease in the ability of the standard CAR T cells to express the IL-18Rα. On the contrary, the CAR bearing CD3-C with the 1XX mutations and the CAR that inducibly expressed IL-18, continued to express the IL-18 receptor alpha. FIG. 2A showed the amount of IL-18Rα expressed when target cells were co-cultured with a T cell transduced to express a CAR with a 41BB co-stimulatory domain and an NFAT-inducible IL18. FIG. 2B showed the amount of IL-18Rα expressed when target cells were co-cultured with a T cell transduced to express a CAR with a CD28 co-stimulatory domain and the 1XX mutations in CD3ζ that were designed to slow signaling to increase in vivo persistence. FIG. 2C showed the amount of IL-18Rα expressed when target cells were co-cultured with a T cell transduced to express a CAR with a 41BB co-stimulatory domain and a wild-type CD32. FIG. 2D showed the amount of IL-18Rα expressed when target cells were co-cultured with a T cell transduced to express a CAR with a CD28 co-stimulatory domain and a wild-type CD32. The experiment showed that the CAR with inducible IL-18 and the CAR with the 1XX mutations expressed 2- to 3-times as much of the IL-18Rα. In addition, the CAR with inducible IL-18 and the CAR with the 1XX mutations did not experience down regulation of IL-18Rα as a consequence of repeated antigen stimulation.

### Example 3. Ability to kill cancer cells expressing low targeted antigen by CAR constructs

Experiments were performed to detect killing ability of cancer cells expressing targeted antigen by CAR constructs with a 41BB co-stimulatory domain and an NFAT-inducible IL-18 or with a CD28 co-stimulatory domain and 1XX mutations in CD32. Target cancer cells were T47D breast cancer cells, wherein the wild-type cells had been engineered to fluoresce red, while a sub-population of 5% of total cells was engineered to express even more MUC1* and made to fluoresce green. Target cancer cells had been cocultured with T cells which had been transduced with various CAR constructs. Four CAR T cells, MNC2-41BB-3z, MNC2-41BB-IL18, MNC2-28-3z and MNC2-28-1XX were antigen stimulated for 6 days. Antigen stimulation involved a synthetic MUC1* extra cellular domain peptide that was covalently coupled to magnetic beads. The CAR T cells and magnetic beads were in RPMI media and media was changed every 48 hours. The magnetic bead to T cell ratio was 10:1. After 6 days antigen stimulation, the MUC1* presenting magnetic beads were removed and the CAR T cells were separately co-cultured with T47D breast cancer cells where 5% of the population had been engineered to express even more MUC1*. These heterogeneous cancer cell populations were co-cultured with various CAR T cell constructs. All the CARs were targeted to the tumor cells by the humanized MNC2-scFv that binds to MUC1*. Photographs were taken at 24, 48 and 72 hours.

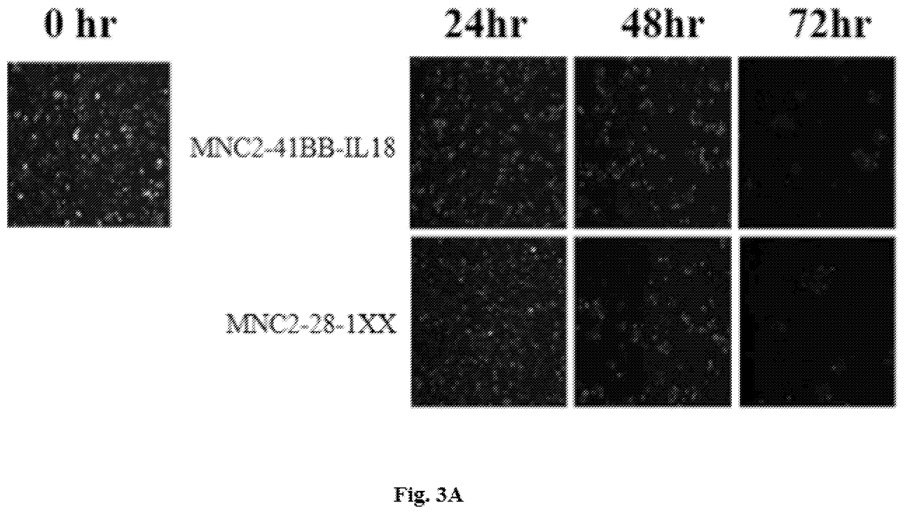

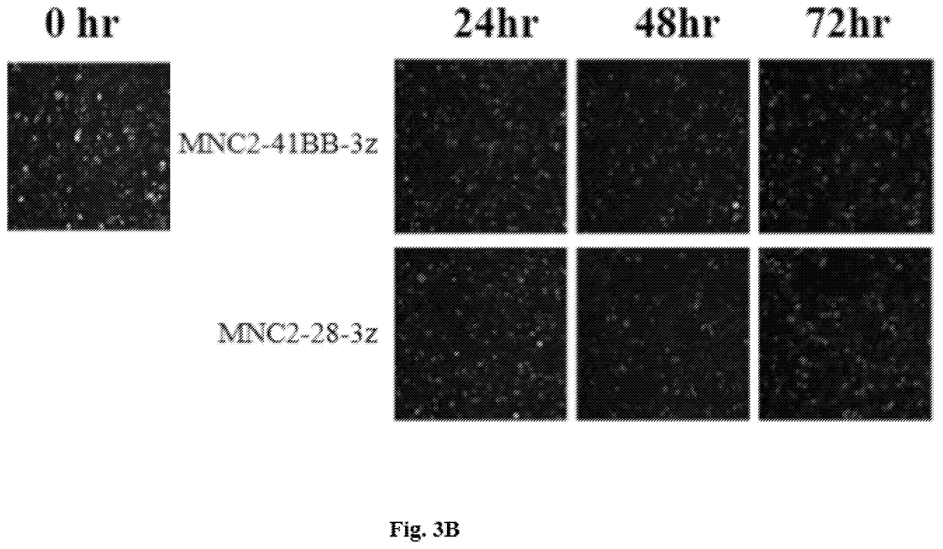

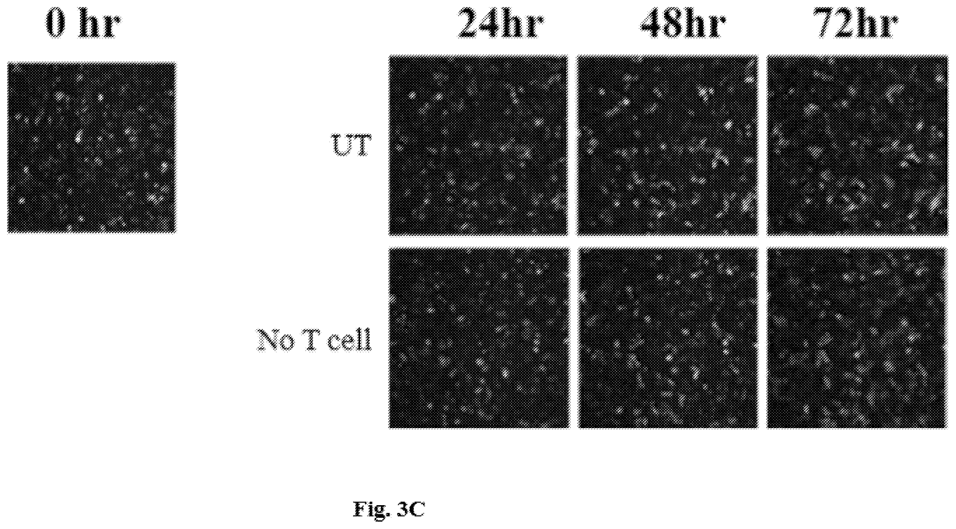

Results from the MNC2-41BB CAR T cells that had a 41BB co-stimulatory domain and bearing an NFAT inducible IL-18 cytokine were shown in FIG. 3A. Also shown in FIG. 3A was the activity of MNC2-28-1XX CAR T cells that have a CD28 co-stimulatory domain and bear the 1XX Tyr to Phe mutations in ITAMs 2 and 3 of CD35. Results of the controls for the CAR T cells shown in FIG. 3A were displayed in FIG. 3B. The reduced killing potency of MNC2-41BB CAR T without the inducible IL-18 cytokine and the reduced killing potency of MNC2-28-3z, wherein CD3° C. was wild-type and without the 1XX mutations were illustrated in FIG. 3B. The control was untransduced cells and CAR with no T cell added as shown in FIG. 3C.

The experiment showed that the high antigen, green cells were quickly killed off, but the low antigen red cells were more resistant to CAR T cell killing. However, the NFAT-inducible IL-18 CAR Ts and the CAR with the 1XX mutations in CD3 killed off the low antigen cells more quickly and more completely than the other two standard CARs. As can be seen in FIGS. 3A-3B, the CARs with the wild-type CD3 stopped killing the low antigen red cells, once the high expressing green cells had been killed, which was known as bystander killing. In contrast, MNC2-41BB-IL18 T cells and MNC2-28-1XX T cells continued to kill the low antigen expressing cells well after the high expressing green cells were gone. At 72 hours post co-culture, only these two CAR T cells had killed all the high and low antigen expressing cancer cells. Taken together, these experiments indicated that it was the maintained expression of the IL-18Rα that was the mechanism of action behind the ability of both CARs with 1XX mutations in CD3ζ and CARs co-expressing IL-18 that increased persistence and enabled the killing of low antigen cells.

FIG. 3A-3C shows the ability of the CAR constructs with an NF AT-inducible IL18 or with the 1XX mutations in CD3ζ to kill target cells that express low levels of the target antigen more quickly and more completely than the other two standard CARs (those with wild-type CD3ζ having either a 41BB or a CD28 co-stimulatory domain) which are able to kill high antigen-expressing target cells. CAR T cells are better able to recognize and kill cells that express high levels of the target antigen, which in this case are the green cells. However, the low antigen expressing red cells are much harder to kill. As can be seen in the figure, the CARs with the wild-type CD3ζ stop killing the low antigen red cells, once the high expressing green cells have been killed, which is known as bystander killing. In contrast, MNC2-41BB-IL18 T cells and MNC2-28-1XX T cells continue to kill the low antigen expressing cells well after the high expressing green cells are gone. At 72 hours post co-culture, only these two CAR T cells have killed all the high and low antigen expressing cancer cells.

### Example 4. Expression of IL-18 Receptor Alpha by Expi CHO Cells Transfected with a Plasmid Encoding a CAR and IL-18Rα

Flow cytometry experiments verifying and quantifying the amount of IL-18 receptor alpha expressed by an Expi-CH O cell transduced expressing a humanized MNC2-CAR with an NFAT inducible IL-18Rα was performed. Expi CHO cells were transfected with the plasmid using ExpiFectamine CHO transfection kit according to manufacturer instructions. In one case, the MNC2 CAR has a CD28 co-stimulatory domain, a CD32-wt, and an NFAT inducible IL-18Rα. In another case, the MNC2 CAR has a CD28 co-stimulatory domain and a CD3ζ with the 1XX mutations, and an NFAT-inducible IL-18Rα. The control cells were non-transfected cells. All elements were encoded on the same plasmid containing a self-cleavage T2A motif so that the CAR and the IL-18Rα were separately expressed on the cell surface. After 3 days, measurements were taken when cells were resting or after activation with PMA [10 ng/mL] and ionomycin [2 uM] for 18 hours. Level of IL18 receptor alpha was measured by flow cytometry using anti Human IL-18Rα alpha conjugated to Alexa 647.

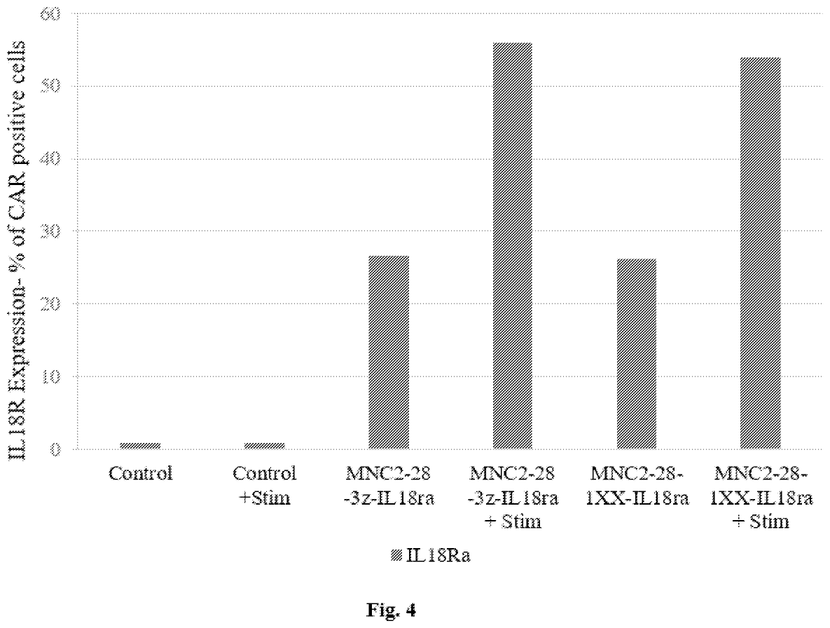

As shown in FIG. 4, the experiment showed that CAR constructs with NFAT-inducible IL-18Rα were generated and levels of IL-18Rα increased after PMA/ionomycin activation.

### Example 5. Expression of IL-18 receptor alpha by humanized MNC2-CAR

Flow cytometry experiments verifying and quantifying the amount of IL-18 receptor alpha and IL-18 receptor beta expressed by an Expi-CH O cell transduced expressing a plasmid encoding a CAR plus IL-18Rα and IL-18Rβ were performed. Expi CHO cells were transfected with the plasmid using ExpiFectamine CHO transfection kit according to manufacturer instructions. The cells were engineered to express a humanized MNC2-CAR with an NFAT inducible IL-18Rα and IL-18Rβ. In one case, the MNC2 CAR had a CD28 co-stimulatory domain, a CD32-wt, and an NFAT inducible IL-18Rα and IL-18Rβ. In another case, the MNC2 CAR had a CD28 co-stimulatory domain and a CD3ζ with the 1XX mutations, and an NFAT-inducible IL-18Rα and IL-18Rβ. The control cells were non-transfected cells. All elements were encoded on the same plasmid containing a self-cleavage T2A motif so that the CAR and the IL-18Rα and IL-18Rβ were separately expressed on the cell surface. After 3 days, measurements were taken when cells were resting or after activation with PMA [10 ng/ml] and ionomycin [2 uM] for 18 hours. Level of IL18 receptor alpha and beta was measured by flow cytometry using anti Human IL-18 Ra and IL-18Rβ.

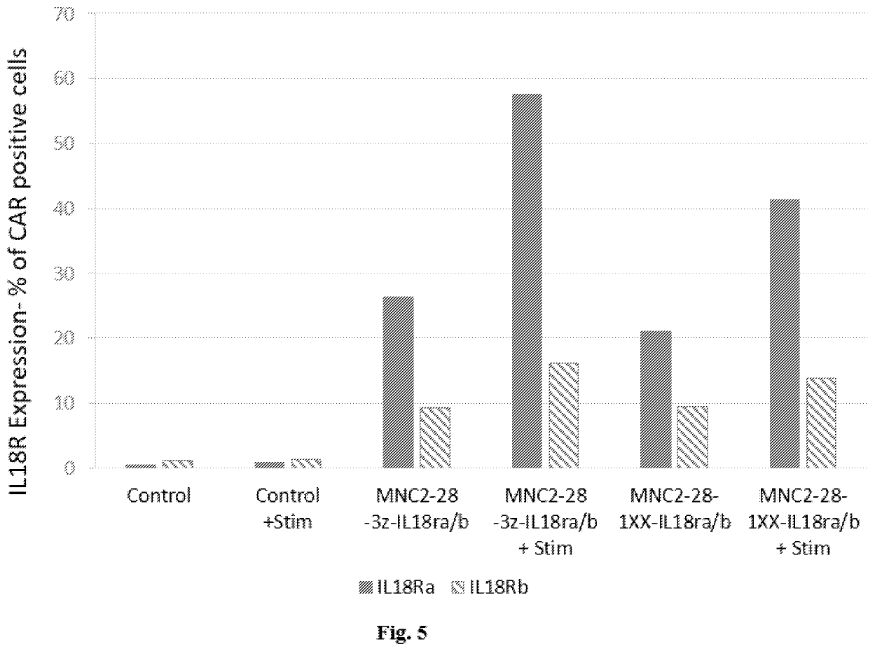

Results as illustrated in FIG. 5, showed that expression level of IL-18 receptor alpha was increased by about 2-fold after humanized MNC2-CAR with an NFAT inducible IL-18Rα and IL-18Rβ was activated by PMA/ionomycin. IL-18Rβ expression increased by about 1.5-fold after PMA/ionomycin stimulation. Compared to the control cells, expression of IL-18Rα was increased by about 50-fold and IL-18Rβ was increased by about 10-fold. The experiment shows that the CAR constructs with NFAT-inducible IL-18Rα and IL-18Rβ expressed the IL-18Rα and IL-18Rβ. The expression was mediated by activated NF AT.

### Example 6. Expression of IL-18Rα by CAR T cells translocating NFATs to the nucleus

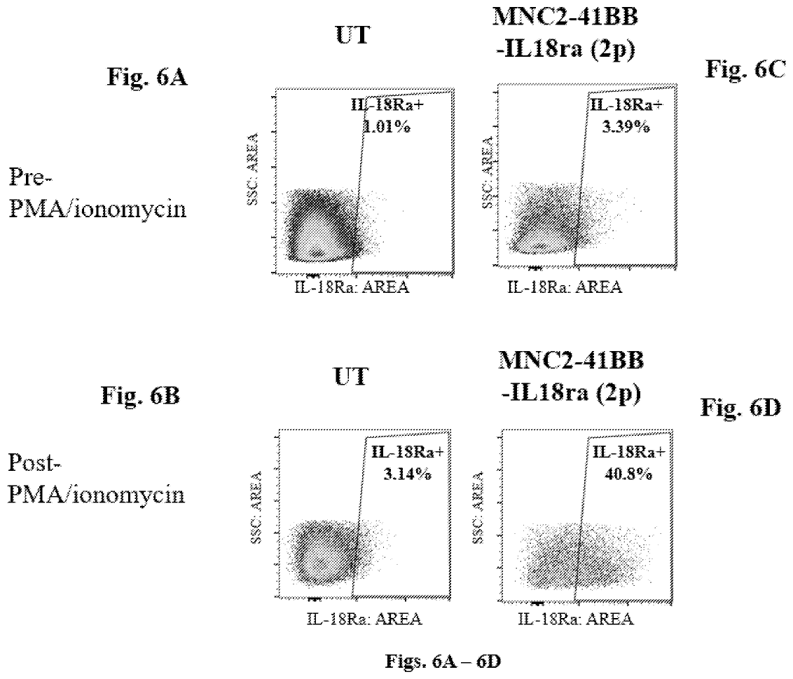

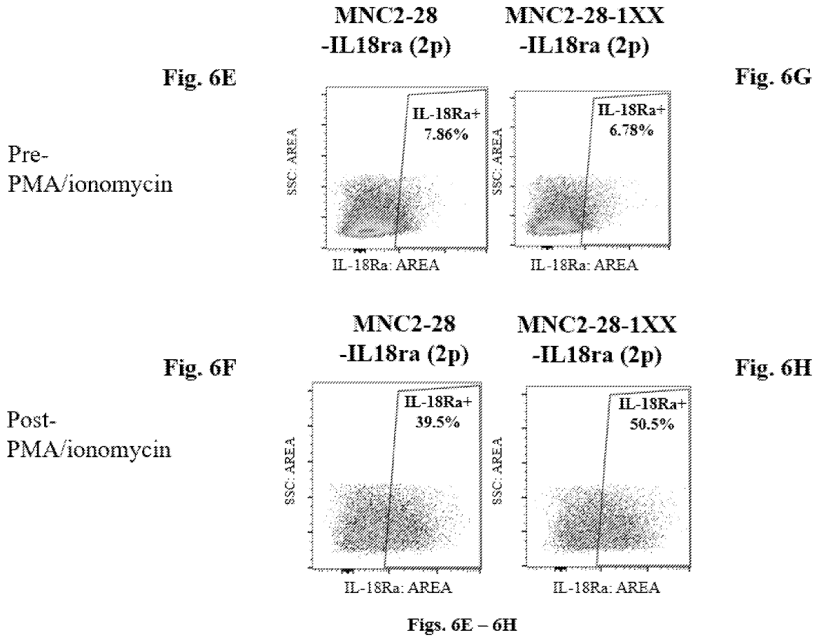

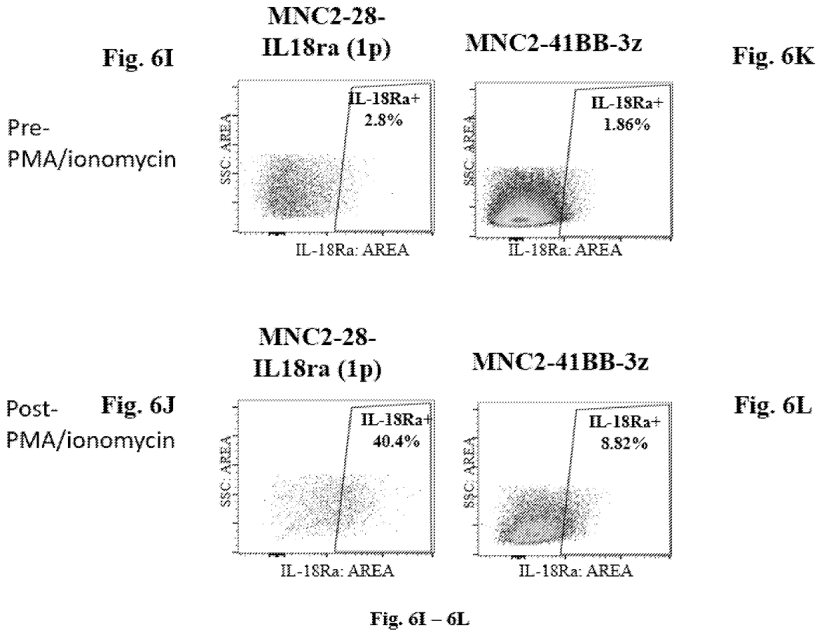

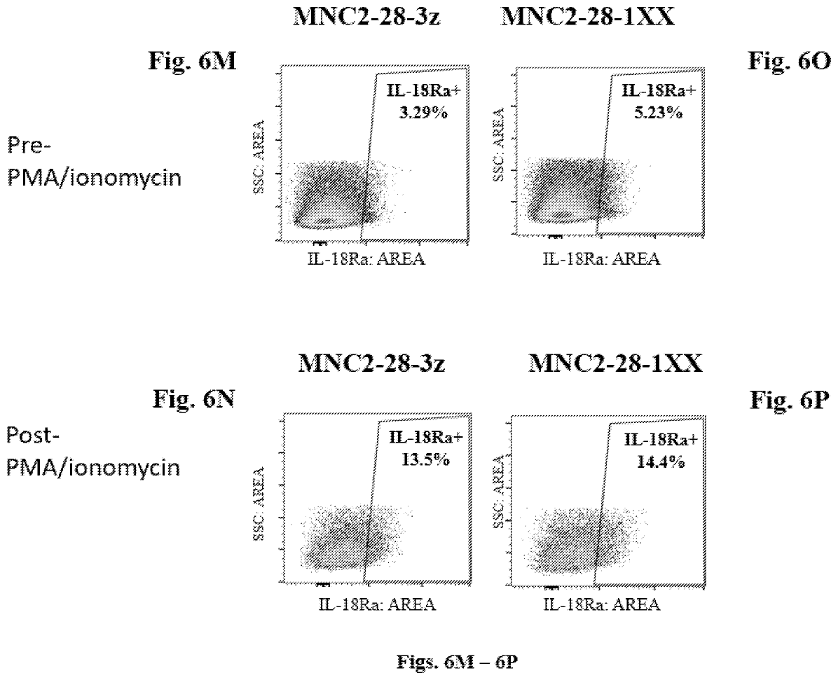

Flow cytometry experiments detecting expression of IL-18Rα in various CAR constructs in transduced donor T cells, before and after stimulation with PMA/ionomycin, were performed. Inducible IL-18Rα and CAR were on the same vector (1p) or separate vectors (2p). Day 9 T cells were stimulated with 10 ng/mL PMA+2 uMionomycin for about 18 hours. IL-18Rα gate was set at 1% in non-stimulated untransduced cells. Quantified results of flow cytometry experiments detecting expression of CAR constructs in transduced donor T cells, before and after stimulation with PMA/ionomycin were shown in FIGS. 6A-6P show flow cytometry scatter plots measuring the amount of IL-18Rα expressed by T cells transduced with various CAR constructs, before and after activation with PMA/ionomycin that translocates NFATs to the nucleus. FIGS. 6A, C, E, G, I, K, M, and O show FACS plot measuring IL18Rα expression before PMA/ionomycin activation. FIGS. 6B, D, F, H, J, L, N, P show FACS plot measuring IL18Rα expression after PMA/ionomycin activation. FIGS. 6A and 6B show plots for untransduced T cells (UT). FIGS. 6C and 6D show plots for MNC2-41BB-IL-18ra, expressed from 2 separate viral vectors. FIGS. 6E and 6F show plots for MNC2-28-IL-18ra, expressed from 2 separate viral vectors. FIGS. 6G and 6H show plots for MNC2-28-1XX-IL-18ra, expressed from 2 separate viral vectors. FIGS. 61 and 6J show plots for MNC2-28-IL-18ra, expressed from 1 viral vector. FIGS. 6K and 6L show plots for MNC2-41BB. FIGS. 6M and 6N show plots for MNC2-28. FIGS. 60 and 6P show plots for MNC2-28-1XX. As can be seen in the figure, after activation by PMA/ionomycin, T cells bearing CARs plus inducible IL-18Rα express 2 to 2.5-times more IL-18Rα expression compared to T cells expressing the CAR alone. T cells bearing a CAR with the 1XX mutations in CD33 and an inducible IL-18Rα, express about 3-times more IL-18Rα expression compared to T cells expressing the CAR alone.

### Example 7. Expression of NFAT Inducible IL-18 Receptor Alpha by CAR Constructs and PMA/Ionomycin Stimulation

Flow cytometry experiments were performed to detect expression of IL-18Rα in various CAR constructs in transduced donor T cells, before and after stimulation with PMA/ionomycin. Inducible IL-18Rα and CAR were on the same vector (1p) or separate vectors (2p). Here, the inducible IL18Rα was expressed from two viral vectors or one, comprising either 6 NFAT response elements or 3 NFAT response elements. Day 9 T cells were stimulated with 10 ng/mL PMA+2 uMionomycin for ˜ 18 hours. IL-18Rα gate was set at 1% in non-stimulated untransduced cells.

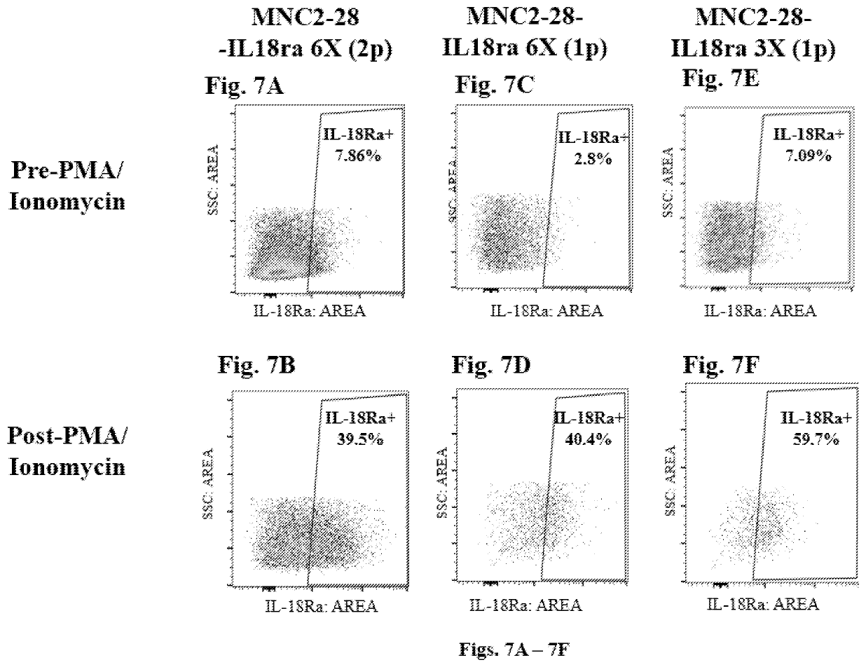

Quantified results of flow cytometry experiments detecting expression of CAR constructs in transduced donor T cells, before and after stimulation with PMA/ionomycin were shown in FIGS. 7A-7F.

FIGS. 7A-7F shows flow cytometry measurement of the expression of an NFAT inducible IL-18 receptor alpha, IL18Rα, before and after activation with PMA/ionomycin, which translocates NFATs to the nucleus. FIG. 7A, FIG. 7C and FIG. 7E show flow plots before PMA/ionomycin activation. FIG. 7B, FIG. 7D, and FIG. 7F show flow plots after PMA/ionomycin activation. FIG. 7A-7B show measurement of IL18Rα for cells transduced with MNC2-CD28 with an inducible IL18Rα, expressed from two viral vectors comprising 6 NF AT response elements. FIG. 7C-7D show measurement of IL18Rα for cells transduced with MNC2-CD28 with an inducible IL18Rα, expressed from one viral vector comprising 6 NFAT response elements. FIG. 7E-7F show measurement of IL18-Ra for cells transduced with MNC2-CD28 with an inducible IL18Rα, expressed from one viral vector comprising 3 NFAT response elements.

### Example 8. CARs with Wild-Type CD3ζ Get Exhausted after Repeated Antigen Stimulation

In this experiment, T cells transduced with MNC2-CAR28-3z or MNC2-CAR28-1XX were repeatedly stimulated for 6 days using magnetic beads to which MUC1* peptides were covalently attached. xCELLigence real-time killing assay was then performed where the CAR T cells were incubated with T47D breast cancer cells of wild-type or had been doped with 5%, 10% or 30% T47D cells to be engineered expressing more MUC1*. Untransduced T cells were included, as were MUC1 negative HEK293 cells.

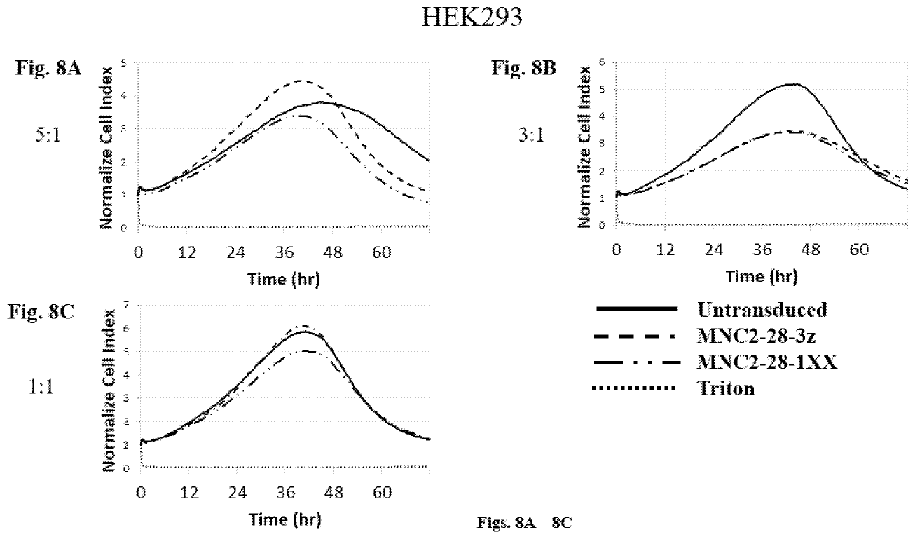

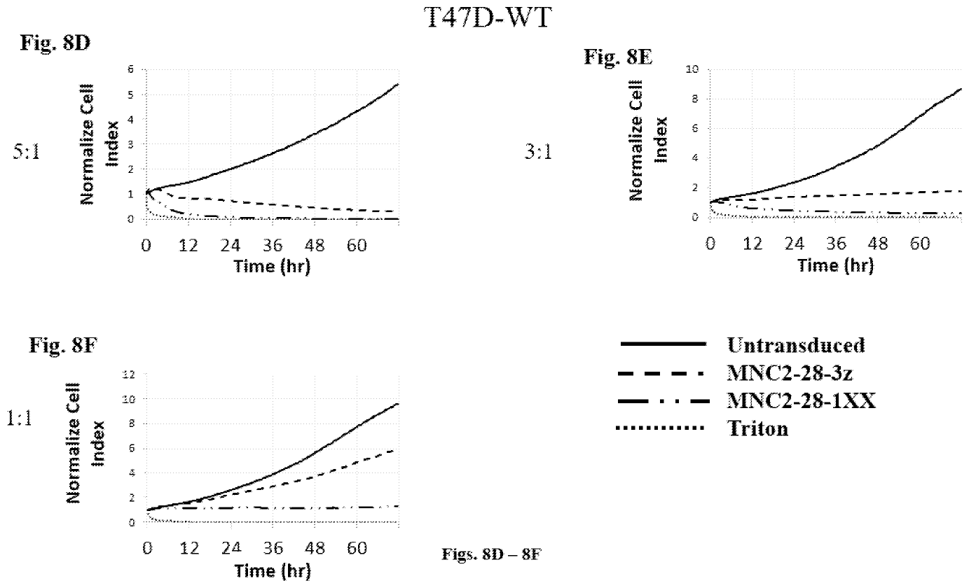

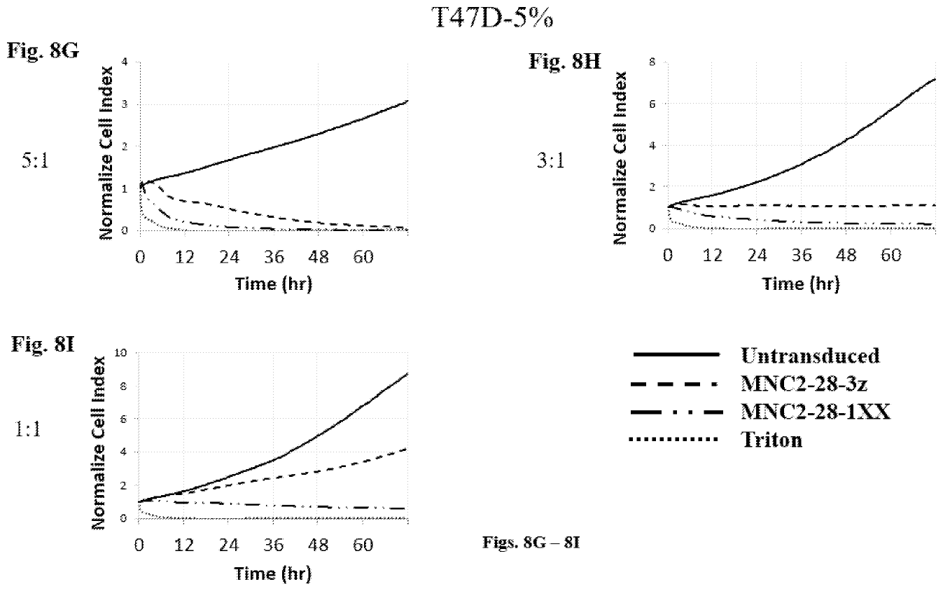

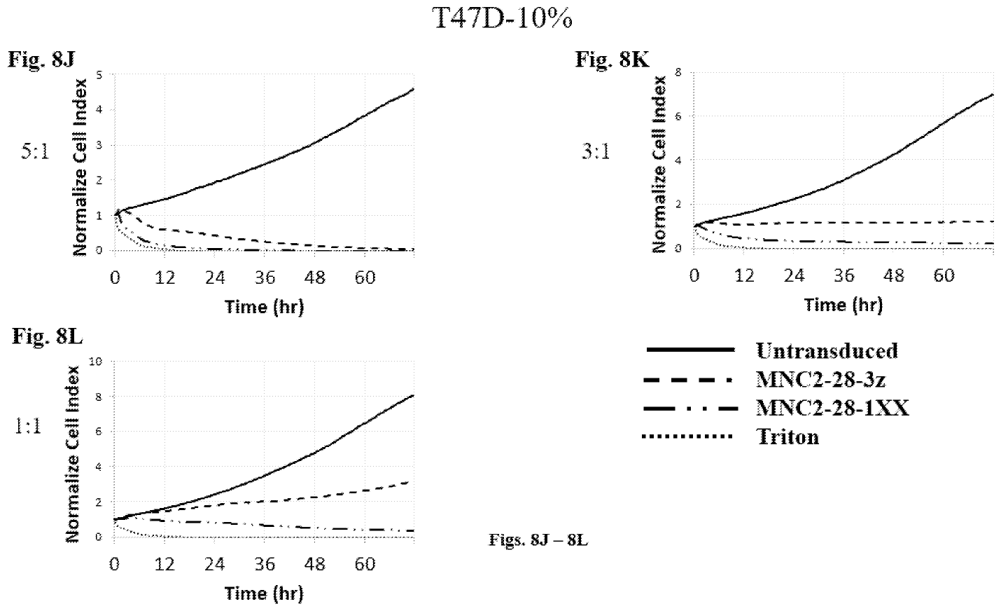

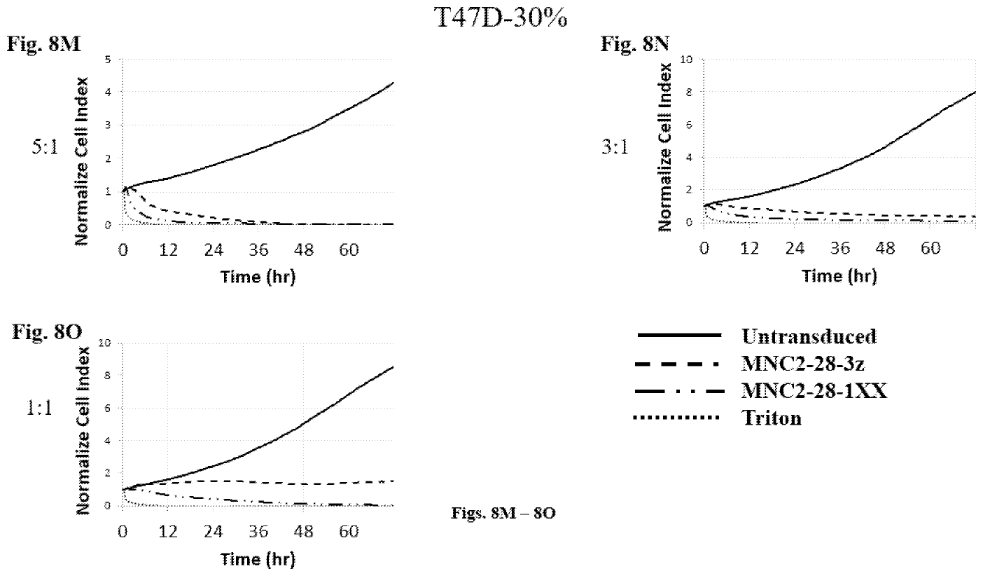

FIGS. 8A-8O show graphs of real-time killing taken on an xCELLigence instrument after 6 days of antigen stimulation. FIG. 8A-8C show xCELLigence graphs where target cells are the control cells, HEK293 that are MUC1/MUC1* negative cells. FIG. 8D-8F show xCELLigence graphs of MNC2-28-3z T cells or MNC2-28-1XX T cells co-cultured with T47D wild-type (-wt) breast cancer cells. FIG. 8G-8I show xCELLigence graphs of MNC2-28-3z T cells or MNC2-28-1XX T cells co-cultured with T47D breast cancer cells in which the population has been doped with 5% of the cells that were engineered to express more MUC1*. FIG. 8J-8L show xCELLigence graphs of MNC2-28-3z T cells or MNC2-28-1XX T cells co-cultured with T47D breast cancer cells in which the population has been doped with 10% of the cells that were engineered to express more MUC1*. FIG. 8M-8O show xCELLigence graphs of MNC2-28-3z T cells or MNC2-28-1XX T cells co-cultured with T47D breast cancer cells in which the population has been doped with 30% of the cells that were engineered to express more MUC1*. The ratio of CAR T cells to cancer cells is marked in the figure as 5:1, 3:1 or 1:1. As can be seen in the figure, after 6 days of antigen stimulation, the diminished killing potency of the CAR bearing a wild-type CD3ζ is evident when either the antigen expression is low, as in FIG. 8D-8F, or the Effector to Target ratio (E:T) is low, or both as in FIG. 8F. In contrast the CAR bearing the 1XX mutations in CD3, in which IL18Rα remains highly expressed even after repeated antigen stimulation, has enhanced ability to kill low antigen expressing cells even at low E:T ratios.

### Example 9. Quantification of Percent CAR Positivity and MFI Across Multiple CAR Constructs

In these experiments, we used flow cytometry to measure the percent CAR positive T cells and their mean fluorescence intensity (MFI). The percent of the T cell population that expresses the CAR as well as how many CARs are expressed on each CAR+ cell, reflected in the MFI, directly affects the killing potential of that population of cells. The results of flow cytometry showed that the percent of CAR-positive T cells varied among the different constructs as shown in Table 9 and Table 10, below.

TABLE 9
CAR expression, percent and mean fluorescence
intensity, measured by flow cytometry

| | % CAR+ Expression | | MFI | |
|---|---|---|---|---|
| Condition | Pre-Stim | Post-Stim | Pre-Stim | Post-Stim |
| UT | 4.6% | 4.3% | 334 | 362 |
| MNC2-41BB-3z | 82.3% | 90.7% | 4,918 | 8,008 |
| MNC2-41BB-3z-IL-18rα | 52.1% | 65.6% | 2,465 | 2,369 |
| MNC2-28-1XX | 70.2% | 92.7% | 3,109 | 10,104 |
| MNC2-28-1XX-IL-18rα | 31.5% | 57.7% | 1,343 | 3,174 |
| MNC2-28-3z | 59.5% | 85.1% | 2,049 | 6,543 |
| MNC2-28-3z-IL-18rα | 44.1% | 70.9% | 1,842 | 4,331 |

TABLE 10
IL-18Rα measured by flow cytometry in CAR-positive cells

| | % IL-18Rα+ Expression | | MFI | |
|---|---|---|---|---|
| | Pre- | Post- | Pre- | Post- |
| Condition | Stim | Stim | Stim | Stim |
| UT* | 2.0% | 15.5% | 886 | 1,183 |
| MNC2-41BB-3z | 4.0% | 14.3% | 958 | 1,198 |
| MNC2-41BB-3z-IL-18ra | 5.4% | 24.8% | 992 | 1,384 |
| MNC2-28-1XX | 7.0% | 15.7% | 1,012 | 1,211 |
| MNC2-28-1XX-IL-18ra | 7.3% | 24.7% | 1,010 | 1,425 |
| MNC2-28-3z | 5.6% | 15.5% | 979 | 1,196 |
| MNC2-28-3z-IL-18ra | 11.6% | 24.8% | 992 | 1,360 |

*% IL-18Rα Positivity and MFI are calculated from the CAR+ population for all conditions except untransduced - in this case, values are calculated from the CD3+ population.

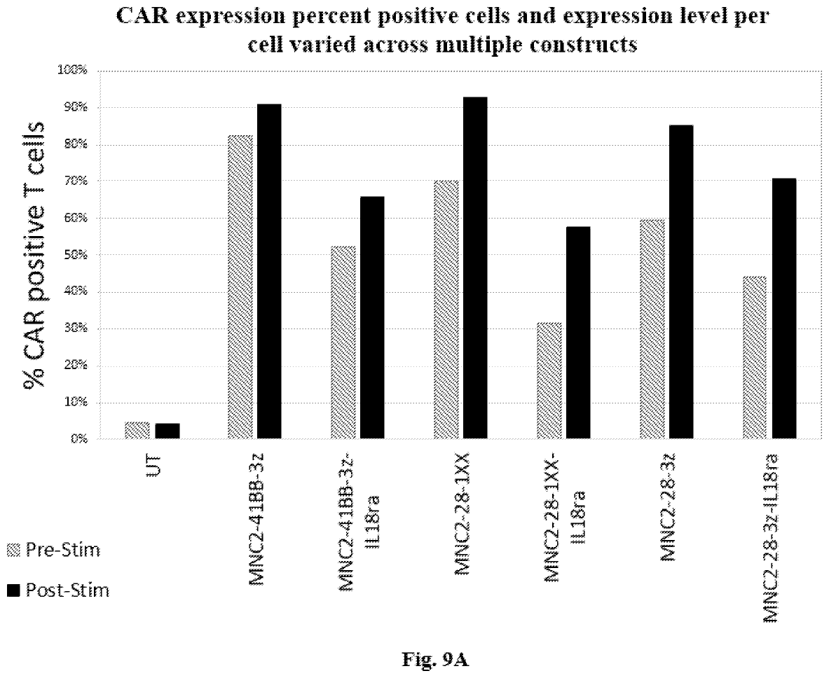

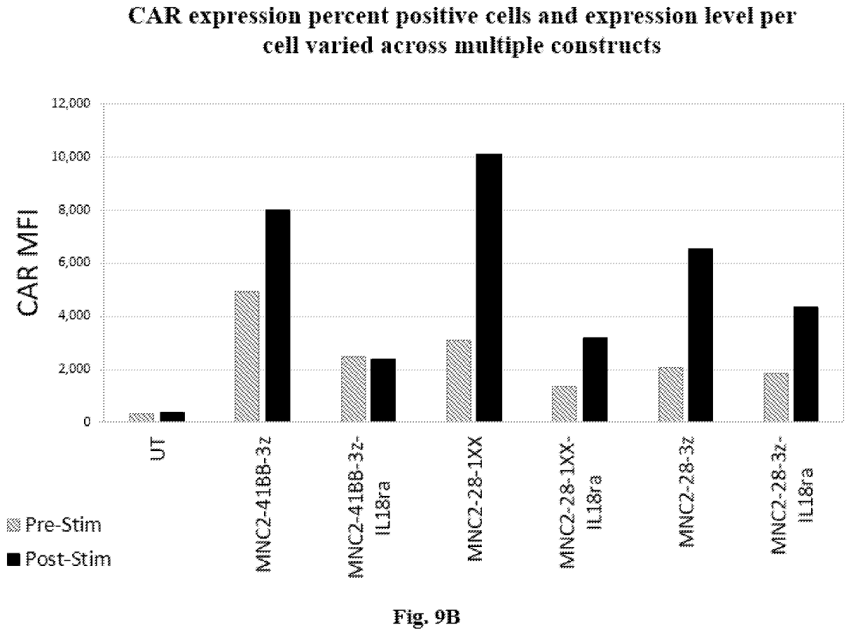

Results are shown in FIG. 9A-9B. FIGS. 9A-9B show bar graphs of flow cytometry measuring the amount of CAR expressed by each construct, before and after the 6 day repeated antigen stimulation. FIG. 9A shows bar graph of flow cytometry measuring the percentage of the T cells that are CAR-positive for each construct, pre- and post-antigen stimulation. FIG. 9B shows bar graph of flow cytometry measuring the mean fluorescence intensity (MFI) of CAR expression for each of the constructs, pre- and post-antigen stimulation. MFI was measured only for the CAR-positive T cell population, so reflects the number of CARs expressed on each cell.

As is shown in FIG. 9A, the percent CAR-positivity was lower in T cells transduced with both a CAR and an inducible IL-18Rα. To normalize, before performing co-culture experiments with target cancer cells, T cells expressing all constructs were diluted with untransduced T cells such that they all had the same percent CAR-positivity as the construct with the lowest percent, 34.8%, which was that of MNC2-28-3z-IL-18Rα. As expected, the percent CAR-positivity increased for all constructs after repeated antigen stimulation. To normalize before co-culture with target cancer cells, CAR T cells were diluted with untransduced T cells to 51.6%, which was the lowest percent CAR-positive construct, MNC2-28-3z-IL-18Rα. However, this normalization scheme does not address the difference in the number of CARs expressed per CAR-positive cell. As in shown in FIG. 9B, the mean fluorescence intensity (MFI), which corresponds to the number of CARs expressed on each CAR-positive cell, is significantly lower in T cells transduced with both a CAR and an inducible IL-18Rα. The higher MFI for T cells transduced with only CAR gives a killing advantage to the T cells expressing the CAR alone.

### Example 10. Assaying the Ability of CAR Constructs to Kill Low Antigen Expressing Cells after Repeated Antigen Stimulation

In these experiments T cells transduced with various CAR constructs were tested for their ability to kill low antigen density cells after repeated antigen stimulation, which is known to induce CAR T cell exhaustion. Here the CAR T cell constructs were MNC2-41BB-CD3z (wild-type) and MNC2-41BB-CD3z (wild-type) with inducible IL-18Rα, notated as MNC2-41BB-CD3z-IL18ra. Antigen stimulation involved a synthetic MUC1* extra cellular domain peptide that was covalently coupled to magnetic beads. The CAR T cells and magnetic beads were in RPMI media and media was changed every 48 hours. The magnetic bead to T cell ratio was 10:1. After 6 days antigen stimulation, the MUC1* presenting magnetic beads were removed and the CAR T cells were separately co-cultured with target expressing cancer cells. IN this example the target cancer cells were T47D breast cancer cells, wherein the wild-type cells had been engineered to fluoresce red, while a sub-population of 5% of total cells was engineered to express even more MUC1* and made to fluoresce green.

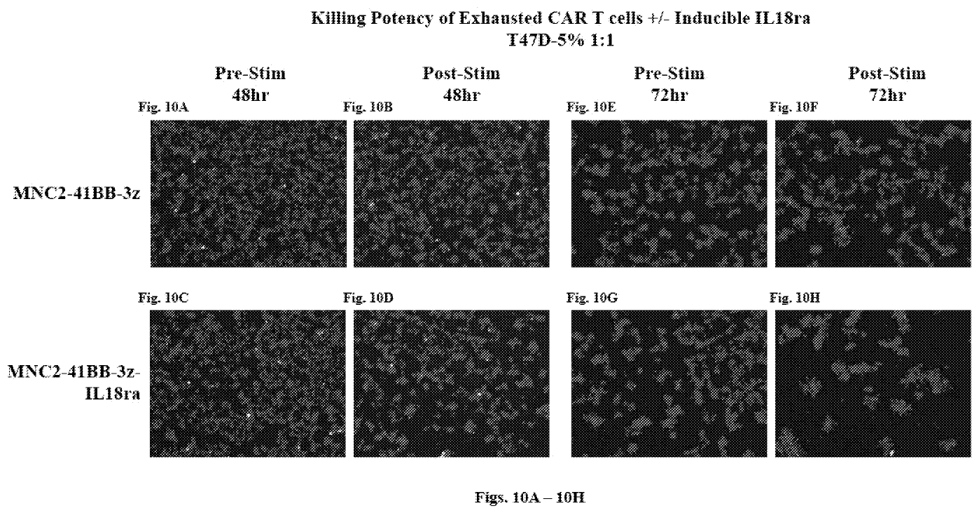

Results are shown in FIG. 10A-10H and in FIG. 11A-11R. FIG. 10A-10H shows fluorescent photographs of two different CAR T cells that have been in co-culture with target cancer cells for either 48 hours or 72 hours. As indicated in the figure, photographs were taken before and after 6 days of repeated antigen stimulation, using magnetic beads to which was attached a synthetic MUC1* extra cellular domain peptide. Here the CAR T cell constructs are MNC2-41BB-CD3z (wild-type) and MNC2-41BB-CD3z (wild-type) with inducible IL-18Rα, notated as MNC2-41BB-CD3z-IL18ra. Here the cancer cells that the CAR T cells were co-cultured with are breast cancer cells, T47D wherein 5% of the population has been engineered to express more MUC1* and engineered to fluoresce green, whereas the wild-type cells fluoresce red. FIG. 10A-10B, and FIG. 10E-10F show photographs that were taken before the 6 days of antigen stimulation. FIG. 10C-10D, and FIG. 10G-10H show photographs that were taken after the 6 days of antigen stimulation, which leads to CAR T cell exhaustion. FIG. 10A-10D show photographs cancers cells after they have been in co-culture with CAR T cells for 48 hours. Note that CAR T cells are present but they have not been engineered to fluoresce. FIG. 10E-10H show photographs cancers cells after they have been in co-culture with CAR T cells for 72 hours.

FIG. 11A-11R shows fluorescent photographs of two CAR T cells that have been in co-culture with target cancer cells for 24, 48 or 72 hours. As indicated in the figure, photographs were taken before and after 6 days of repeated antigen stimulation, using magnetic beads to which was attached a synthetic MUC1* extra cellular domain peptide. Here the CAR T cell constructs are MNC2-41BB-CD3z (wild-type) and MNC2-41BB-CD3z (wild-type) with inducible IL-18Rα, notated as MNC2-41BB-CD3z-IL18ra. Here the cancer cells that the CAR T cells were co-cultured with are breast cancer cells, T47D wherein 5% of the population has been engineered to express more MUC1* and engineered to fluoresce green, whereas the wild-type cells fluoresce red. FIG. 11A-11B, FIG. 11G-11H, and FIG. 11M-11N show photographs of untransduced T cells in co-culture with the cancer cells. FIG. 11C-11D, FIG. 11I-11J, and FIG. 11O-11P show photographs of T cells transduced with CAR MNC2-41BB-CD3z in co-culture with the cancer cells. FIG. 11E-11F, FIG. 11K-11L, and FIG. 11Q-11R show photographs of T cells transduced with CAR MNC2-41BB-CD3z plus an NFAT inducible IL-18Rα in co-culture with the cancer cells. As can be seen in the control wells where untransduced T (UT) cells are present, the cancer cell population contains 95% T47D wild-type cells that fluoresce red and 5% that have been engineered to express more MUC1* that fluoresce green. The photographs taken at 24 hours of co-culture show that both constructs kill the later stage high antigen expressing cells about equally as well. However, the photographs taken at later timepoints shown that the T cells transduced with both a CAR and an inducible IL-18Rα have a superior ability to kill the low antigen expressing cells, characteristic of early cancer cells. It is important to note that the T cells expressing both the CAR and an inducible IL-18Rα have superior ability to kill low antigen expressing cells, despite the fact that there are 30% less CAR+ cells, which express 70% less CARs than T cells expressing the CAR alone. This underscores the enhanced ability of T cells bearing a CAR and an inducible IL-18Rα to kill the low antigen expressing cells as well as their ability to resist CAR T cell exhaustion.

As can be seen in FIG. 10 and FIG. 11, T cells transduced with CAR MNC2-41BB-CD3z plus an NFAT inducible IL-18Rα retain the ability to kill the early, low antigen expressing cancer cells better than the T cells transduced with the CAR alone. These data show that CAR T cells without inducible IL-18Rα get exhausted after 6 days of antigen stimulation and lose ability to kill the early, low antigen expressing cells.

### Example 11. Measuring Differences in Killing Potency Using a Real-Time Killing Assay

In this experiment, an xCELLigence instrument was used to measure killing of target cells by T cells transduced with various CAR constructs. Target cancer cells, which are adherent, are plated onto multi-electrode array plates. The instrument measures conductivity, or rather lack thereof called impedance, through the electrodes. The adherent cancer cells coat the electrode and insulate it, causing a drop in conductivity and a rise in impedance. As cancer cells grow on the electrode, impedance increases. The output is a plot of impedance on the Y-axis as a function of time. T cells are non-adherent, suspension cells so do not contribute to impedance. As CAR T cells kill the cancer cells coating the electrodes, they lyse and a decrease in impedance is measured.

In this experiment, the T cells have been transduced with either MNC2-41BB-CD3z (wild-type) and MNC2-41BB-CD3z (wild-type) with inducible IL-18Rα, notated as MNC2-41BB-CD3z-IL18ra. The CAR T cells have been repeatedly antigen stimulated for 6 days. Here the cancer cells that the CAR T cells were co-cultured with are breast cancer cells, T47D wherein the cells are all wild-type (wt), or doped with 5% or 10% of T47D cells that have been engineered to express more MUC1*. FIG. 12A shows the killing potency of the two types of CAR T cells co-cultured with T47D-wt cancer cells before the repeated antigen stimulation. FIG. 12B shows the killing potency after antigen stimulation. FIG. 12C and FIG. 12D show the killing curves of the two types of CAR T cells co-cultured with T47D-5% cancer cells before and after antigen stimulation. FIG. 12E and FIG. 12F show the killing curves of the two types of CAR T cells co-cultured with T47D-10% cancer cells before and after antigen stimulation.

The figure shows that after 6 days of repeated antigen stimulation, which can lead to CAR T cell exhaustion, the T cells bearing both a CAR and an inducible IL-18Rα have superior killing ability, consistent with their ability to resist CAR T cell exhaustion. As is expected, the difference between the two CAR T cells gets smaller as the antigen density of the target cancer cells increases, which is consistent with bystander killing.

### Example 12. Assaying the Ability of CAR Constructs to Kill Low Antigen Expressing Cells after Repeated Antigen Stimulation

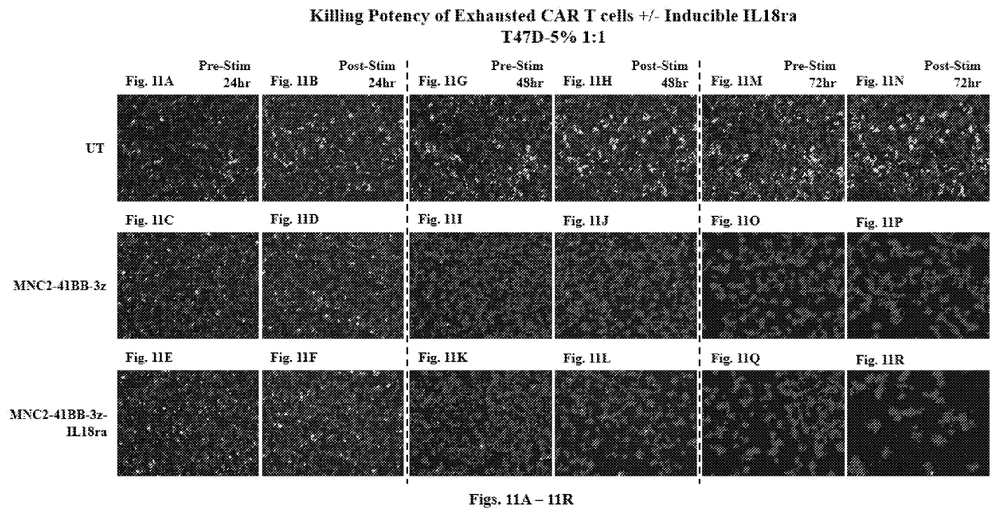

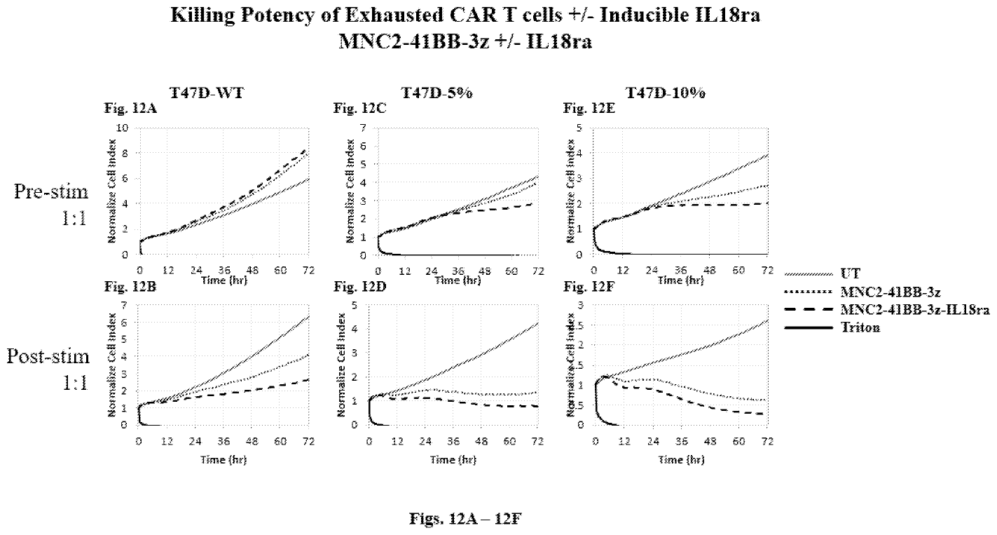

In these experiments T cells transduced with various CAR constructs were tested for their ability to kill low antigen density cells after repeated antigen stimulation, which is known to induce CAR T cell exhaustion. Here the CAR T cell constructs are MNC2-28-CD3z (wild-type) and MNC2-28-CD3z (wild-type) with inducible IL-18Rα, notated as MNC2-28-CD3z-IL18ra. The CAR T cells were antigen stimulated for 6 days prior to co-culture with target expressing cancer cells. Here the cancer cells that the CAR T cells were co-cultured with are breast cancer cells, T47D wherein 5% of the population has been engineered to express more MUC1* and engineered to fluoresce green, whereas the wild-type cells fluoresce red. Results are shown in FIG. 13. FIG. 13A-13R shows fluorescent photographs of two CAR T cells that have been in co-culture with target cancer cells for 24, 48 or 72 hours. As indicated in the figure, photographs were taken before and after 6 days of repeated antigen stimulation, using magnetic beads to which was attached a synthetic MUC1* extra cellular domain peptide. Here the CAR T cell constructs are MNC2-28-CD3z (wild-type) and MNC2-28-CD3z (wild-type) with inducible IL-18Rα, notated as MNC2-28-CD3z-IL18ra. Here the cancer cells that the CAR T cells were co-cultured with are breast cancer cells, T47D wherein 5% of the population has been engineered to express more MUC1* and engineered to fluoresce green, whereas the wild-type cells fluoresce red. FIG. 13A-13B, FIG. 13G-13H, and FIG. 13M-13N show photographs of untransduced T cells in co-culture with the cancer cells. FIG. 13C-13D, FIG. 131-13J, and FIG. 130-13P show photographs of T cells transduced with CAR MNC2-28-CD3z in co-culture with the cancer cells. FIG. 13E-13F, FIG. 13K-13L, and FIG. 13Q-13R show photographs of T cells transduced with CAR MNC2-28-CD3z plus an NFAT inducible IL-18Rα in co-culture with the cancer cells. As can be seen in the control wells where untransduced T (UT) cells are present, the cancer cell population contains 95% T47D wild-type cells that fluoresce red and 5% that have been engineered to express more MUC1* that fluoresce green. The photographs show that, after repeated antigen stimulation, the construct with inducible IL-18Rα, MNC2-28-3z-IL18ra, kills the low antigen expressing cells better than the T cells bearing the CAR alone. Note the difference in killing at 24 hours is especially evident. It is important to note that the T cells expressing both the CAR and an inducible IL-18Rα have superior ability to kill low antigen expressing cells, despite the fact that there are 20% less CAR+ cells, that express 33% less CARs than T cells expressing the CAR alone. This underscores the enhanced ability of T cells bearing a CAR and an inducible IL-18Rα to kill the low antigen expressing cells as well as their ability to resist CAR T cell exhaustion.

### Example 13. Measuring Differences in Killing Potency Using a Real-Time Killing Assay

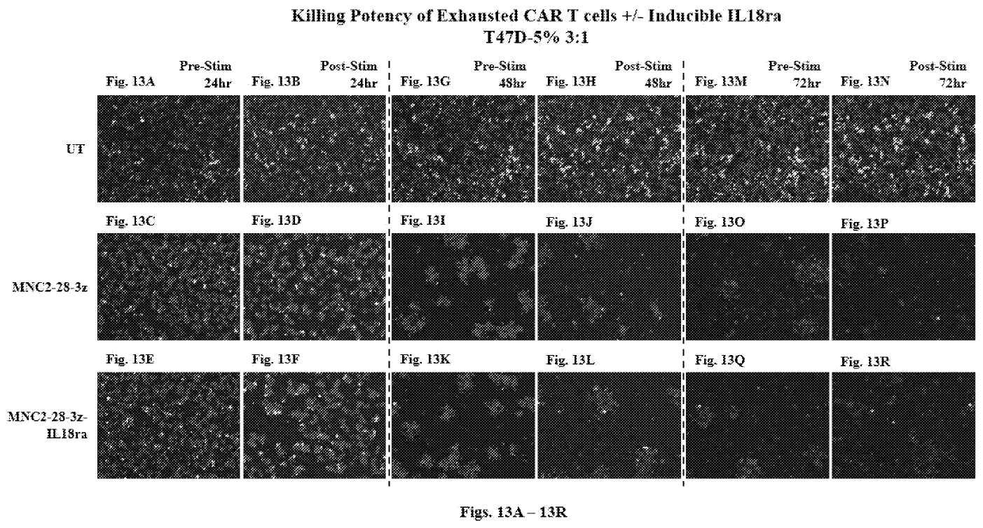

In this experiment, an xCELLigence instrument was used to measure killing of target cells by T cells transduced with various CAR constructs. In this experiment, the T cells have been transduced with either MNC2-28-CD3z (wild-type) and MNC2-28-CD3z (wild-type) with inducible IL-18Rα, notated as MNC2-28-CD3z-IL18ra. Here the cancer cells that the CAR T cells were co-cultured with are breast cancer cells, T47D wherein the cells are all wild-type (wt), or doped with 5% or 10% of T47D cells that have been engineered to express more MUC1*. The CAR T cells have been repeatedly antigen stimulated for 6 days prior to co-culture with target cells. Results are shown in FIG. 14. FIG. 14A shows the killing potency of the two types of CAR T cells co-cultured with T47D-wt cancer cells before the repeated antigen stimulation. FIG. 14B shows the killing potency after antigen stimulation. FIG. 14C and FIG. 14D show the killing curves of the two types of CAR T cells co-cultured with T47D-5% cancer cells before and after antigen stimulation. FIG. 14E and FIG. 14F show the killing curves of the two types of CAR T cells co-cultured with T47D-10% cancer cells before and after antigen stimulation. As can be seen in the figure, before repeated antigen stimulation that can lead to exhaustion, MNC2-28-CD3z kills significantly better than MNC2-28-CD3z-IL18ra. This result is consistent with the fact that MNC2-28-CD3z-IL18ra has are 20% less CAR+ cells, that express 33% less CARs than T cells expressing the CAR alone. However, after antigen stimulation, the killing potency is the opposite, with MNC2-28-CD3z-IL18ra T cells killing better than the T cell expressing the CAR alone.

### Example 14. Assaying the Ability of CAR Constructs to Kill Low Antigen Expressing Cells after Repeated Antigen Stimulation

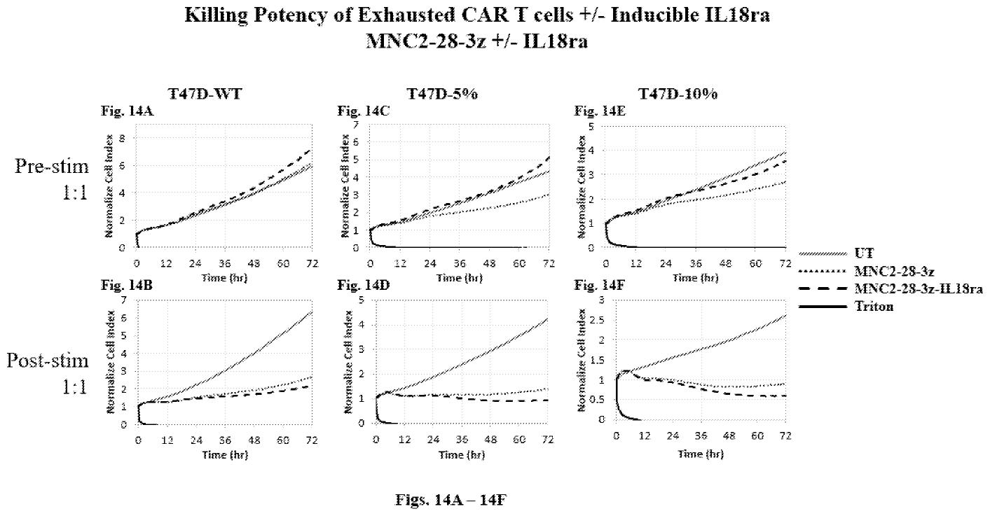

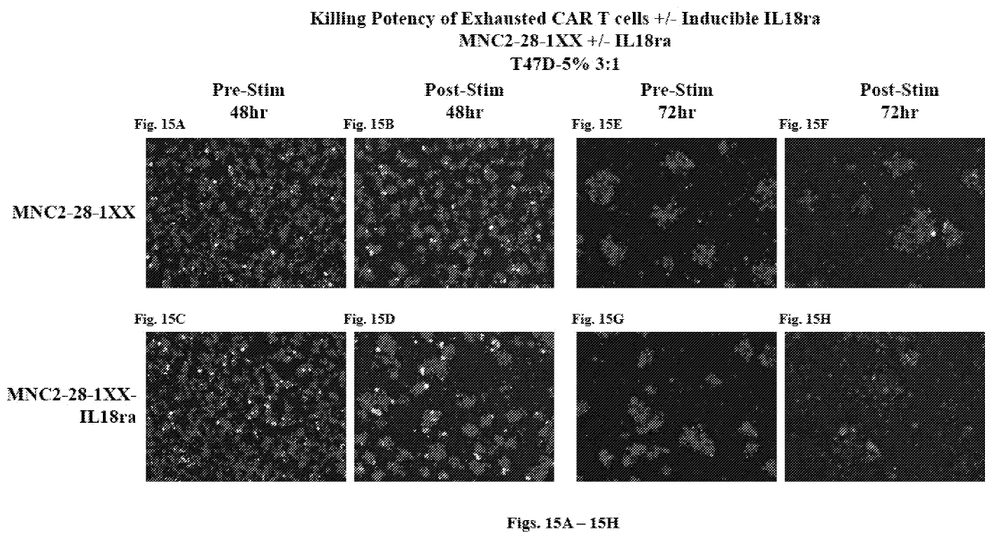

In these experiments T cells transduced with various CAR constructs were tested for their ability to kill low antigen density cells after repeated antigen stimulation, which is known to induce CAR T cell exhaustion. Here the CAR T cell constructs are Here the CAR T cell constructs are MNC2-28-1XX and MNC2-28-1XX with inducible IL-18Rα, notated as MNC2-28-1XX-IL18ra. The CAR T cells were antigen stimulated for 6 days prior to co-culture with target expressing cancer cells. Here the cancer cells that the CAR T cells were co-cultured with are breast cancer cells, T47D wherein 5% of the population has been engineered to express more MUC1* and engineered to fluoresce green, whereas the wild-type cells fluoresce red. Results are shown in FIG. 15A-15H.

FIG. 15A-15H shows fluorescent photographs of two different CAR T cells that have been in co-culture with target cancer cells for either 48 hours or 72 hours. FIG. 15A-15B, and FIG. 15E-15F show photographs of T47D-5% cancer cells co-cultured with MNC2-28-1XX, before and after the 6 days of antigen stimulation. FIG. 15C-15D, and FIG. 15G-15H show photographs of T47D-5% cancer cells co-cultured with MNC2-28-1XX-IL18ra, before and after the 6 days of antigen stimulation, which leads to CAR T cell exhaustion. FIG. 15A-15D show photographs cancers cells after they have been in co-cultured with CAR T cells for 48 hours. FIG. 15E-15H show photographs cancers cells after they have been in co-culture with CAR T cells for 72 hours. As can be seen in FIG. 15A-15H, T cells transduced with CAR MNC2-28-1XX plus an NFAT inducible IL-18Rα retain the ability to kill the early, low antigen expressing cancer cells better than the T cells transduced with the CAR alone. It is notable that MNC2-28-1XX plus an NFAT inducible IL-18Rα kill the early, low antigen expressing cells better than the T cells bearing a CAR alone despite the fact that MNC2-28-1XX-IL18ra has nearly 40% less CAR-positive T cells and the CAR+ cells have nearly 60% less CARs per cell.

### Example 15. Measuring Differences in Killing Potency Using a Real-Time Killing Assay

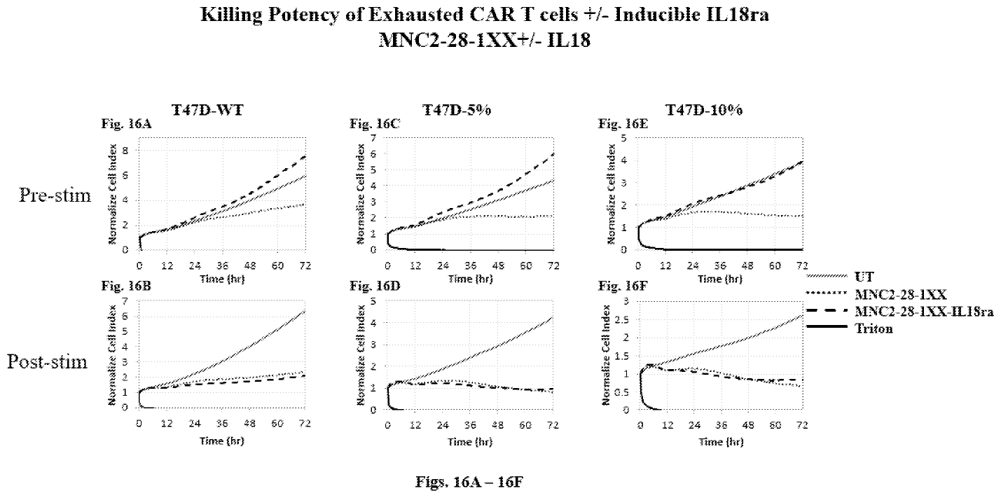

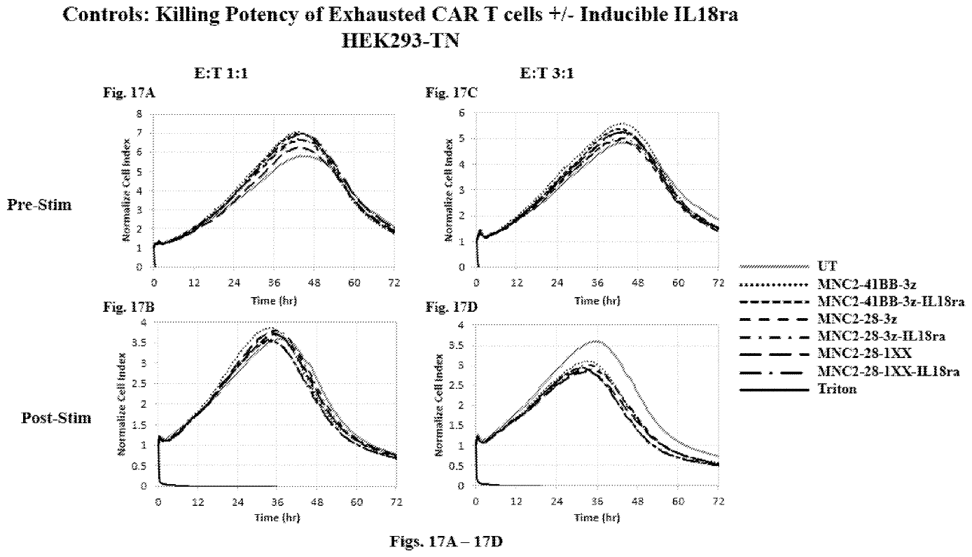

In this experiment, an xCELLigence instrument was used to measure killing of target cells by T cells transduced with various CAR constructs. In this experiment, the T cells have been transduced with either MNC2-28-1XX and MNC2-28-1XX with inducible IL-18Rα, notated as MNC2-28-1XX-IL18ra. The CAR T cells have been repeatedly antigen stimulated for 6 days prior to co-culture with target cancer cells. Here the cancer cells that the CAR T cells were co-cultured with are breast cancer cells, T47D wherein the cells are all wild-type (wt), or doped with 5% or 10% of T47D cells that have been engineered to express more MUC1*. FIG. 16A shows the killing potency of the two types of CAR T cells co-cultured with T47D-wt cancer cells before the repeated antigen stimulation. FIG. 16B shows the killing potency after antigen stimulation. FIG. 16C and FIG. 16D show the killing curves of the two types of CAR T cells co-cultured with T47D-5% cancer cells before and after antigen stimulation. FIG. 16E and FIG. 16F show the killing curves of the two types of CAR T cells co-cultured with T47D-10% cancer cells before and after antigen stimulation. As can be seen in the figure, before repeated antigen stimulation that can lead to exhaustion, MNC2-28-1XX kills significantly better than MNC2-28-1XX-IL18ra. This result is consistent with the fact that MNC2-28-1XX-IL18ra has are 40% less CAR+ cells, that express about 60% less CARs than T cells expressing the CAR alone. However, after antigen stimulation, the killing potency is the opposite, with MNC2-28-1XX-IL18ra T cells killing better than the T cell expressing the CAR alone.

Results of the CAR T cells co-cultured with control, non-target cells are shown in FIG. 17.

FIG. 17A-17D show control experiment xCELLigence real-time killing assay, wherein all CAR T cell constructs were co-cultured with MUC1/MUC1* negative cells, HEK293 cells. FIG. 17A-17B show pre- and post-antigen stimulation killing, or lack thereof, wherein the T cell to HEK293 cell ratio was 1:1. FIG. 17C-17D show pre- and post-antigen stimulation killing, or lack thereof, wherein the T cell to HEK293 cell ratio was 3:1.

1-170. (canceled)

171. A method of treating cancer in a subject in need thereof comprising administering an immune cell to the subject, wherein:
the immune cell comprises:
a first polynucleotide encoding an interleukin-18 (IL-18) receptor, and
a second polynucleotide encoding a chimeric antigen receptor (CAR); and
the cancer is characterized by a tumor comprising cancer cells having heterogeneous expression of a CAR antigen.

172. A method comprising administering an immune cell to a subject, wherein the immune cell comprises:
a first polynucleotide that encodes an interleukin-18 (IL-18) receptor, and
a second polynucleotide that encodes a chimeric antigen receptor (CAR);
thereby increasing persistence of the immune cell in the subject compared to an immune cell not comprising the first polynucleotide and comprising the second polynucleotide.

173. A method of treating cancer in a subject in need thereof comprising administering an immune cell to the subject, wherein:
the immune cell comprises:
a first polynucleotide that encodes an interleukin-18 (IL-18) receptor, and
a second polynucleotide that encodes a chimeric antigen receptor (CAR);
thereby inhibiting recurrence of the cancer.

174. The method of claim 171, wherein the cancer comprises breast cancer, lung cancer, prostate cancer, ovarian cancer, colorectal cancer, or pancreatic cancer.

175. The method of claim 174, wherein expression of the IL-18 receptor is induced when the CAR is stimulated by the CAR antigen.

176. The method of claim 175, wherein the first polynucleotide is operably linked to a nuclear factor of activated T-cells (NFAT) response element.

177. The method of claim 176, wherein the NFAT response element is from an IL-2 promoter region or a FOXP3 promoter region.

178. The method of claim 171, wherein the first polynucleotide comprises a polynucleotide sequence with at least 90% identity to SEQ ID NO: 26, SEQ ID NO: 27, SEQ ID NO: 28, SEQ ID NO: 29, SEQ ID NO: 30, SEQ ID NO: 31, SEQ ID NO: 32, or SEQ ID NO: 33.

179. The method of claim 171, wherein the CAR comprises a single chain variable fragment (scFv) that binds to MUC1*, wherein the scFv comprises:
a heavy chain (HC) variable domain comprising complementarity determining regions (CDRs) consisting of an HC-CDR1 comprising SEQ ID NO: 37, an HC-CDR2 comprising SEQ ID NO: 38, and an HC-CDR3 comprising SEQ ID NO: 39; and
a light chain (LC) variable domain comprising CDRs consisting of a LC-CDR1 comprising SEQ ID NO: 40, a LC-CDR2 comprising SEQ ID NO: 41, and a LC-CDR3 comprising SEQ ID NO: 42.

180. The method of claim 179, wherein the scFv comprises an amino acid sequence with at least 90% sequence identity to SEQ ID NO: 43 and an amino acid sequence with at least 90% sequence identity to SEQ ID NO: 44.

181. The method of claim 171, wherein the CAR comprises a single chain variable fragment (scFv) that binds to MUC1*, wherein the scFv comprises:
a HC variable domain comprising CDRs consisting of an HC-CDR1 comprising SEQ ID NO: 46, an HC-CDR2 comprising SEQ ID NO: 47, and an HC-CDR3 comprising SEQ ID NO: 48; and
a LC variable domain comprising CDRs consisting of a LC-CDR1 comprising SEQ ID NO: 49, a LC-CDR2 comprising SEQ ID NO: 50, and a LC-CDR3 comprising SEQ ID NO: 51; or
a HC variable domain comprising CDRs consisting of an HC-CDR1 comprising SEQ ID NO: 55, an HC-CDR2 comprising SEQ ID NO: 56, and an HC-CDR3 comprising SEQ ID NO: 57; and
a LC variable domain comprising CDRs consisting of a LC-CDR1 comprising SEQ ID NO: 58, a LC-CDR2 comprising SEQ ID NO: 59, and a LC-CDR3 comprising SEQ ID NO: 60.

182. The method of claim 171, wherein the CAR comprises a hinge region from CD28 and a transmembrane region from CD28.

183. The method of claim 171, wherein the CAR comprises a co-stimulatory domain from CD28.

184. The method of claim 171, wherein the CAR comprises a signaling domain derived from CD3ζ.

185. The method of claim 184, wherein the signaling domain comprises SEQ ID NO: 71.

186. The method of claim 179, wherein the CAR comprises a hinge region comprising SEQ ID NO: 65, a transmembrane domain comprising SEQ ID NO: 67, a co-stimulatory domain comprising SEQ ID NO: 69, and a signaling domain comprises SEQ ID NO: 71.

187. The method of claim 171, wherein the CAR comprises the amino acid sequence of any one of SEQ ID NOs: 72-83.

188. The method of claim 171, wherein a vector comprises the first polynucleotide and the second polynucleotide.

189. The method of claim 188, wherein the vector comprises a polynucleotide sequence having at least 90% identity to any one of SEQ ID NOs: 96-190.

190. The method of claim 171, wherein the immune cell is a T cell.

191. The method of claim 190, wherein the immune cell is autologous to the subject.

192. The method of claim 190, wherein the immune cell is allogeneic to the subject.

193. A vector comprising:
a first polynucleotide encoding an interleukin-18 (IL-18) receptor, and
a second polynucleotide encoding a chimeric antigen receptor (CAR).

194. The vector of claim 193, wherein the vector comprises a polynucleotide sequence of any one of SEQ ID NOs: 96-190.

195. The vector of claim 193, wherein the vector further comprises a polynucleotide sequence that targets integration of the second polynucleotide into a T-cell receptor α constant (TRAC) locus.

196. An immune cell comprising the vector of claim 193.

197. A pharmaceutical composition comprising the immune cell of claim 196, and a pharmaceutically acceptable excipient.

198. An immune cell comprising:
a first polynucleotide encoding an interleukin-18 (IL-18) receptor, and
a second polynucleotide encoding a chimeric antigen receptor (CAR);
wherein the CAR comprises an scFv that binds to MUC1*, and the scFv comprises:
an HC-CDR1 comprising SEQ ID NO: 37, an HC-CDR2 comprising SEQ ID NO: 38, an HC-CDR3 comprising SEQ ID NO: 39, a LC-CDR1 comprising SEQ ID NO: 40, a LC-CDR2 comprising SEQ ID NO: 41, and a LC-CDR3 comprising SEQ ID NO: 42;
an HC-CDR1 comprising SEQ ID NO: 46, an HC-CDR2 comprising SEQ ID NO: 47, an HC-CDR3 comprising SEQ ID NO: 48, a LC-CDR1 comprising SEQ ID NO: 49, a LC-CDR2 comprising SEQ ID NO: 50, and a LC-CDR3 comprising SEQ ID NO: 51; or
an HC-CDR1 comprising SEQ ID NO: 55, an HC-CDR2 comprising SEQ ID NO: 56, an HC-CDR3 comprising SEQ ID NO: 57, a LC-CDR1 comprising SEQ ID NO: 58, a LC-CDR2 comprising SEQ ID NO: 59, and a LC-CDR3 comprising SEQ ID NO: 60.


SEQUENCE LISTING (190 sequences)

SEQ ID NO: 1 - protein, 21 aa, synthetic construct
MNCRELPLTLWVLISVSTAES

SEQ ID NO: 2 - protein, 308 aa, synthetic construct
CTSRPHITVVEGEPFYLKHCSCSLAHEIETTTKSWYKSSGSQEHVELNPRSSSRIALHDCVLEFWPVELNDTGSYFFQMKNYTQKWKLNVIRRNKHSCFTERQVTSKIVEVKKFFQITCENSYYQTLVNSTSLYKNCKKLLLENNKNPTIKKNAEFEDQGYYSCVHFLHHNGKLFNITKTFNITIVEDRSNIVPVLLGPKLNHVAVELGKNVRLNCSALLNEEDVIYWMFGEENGSDPNIHEEKEMRIMTPEGKWHASKVLRIENIGESNLNVLYNCTVASTGGTDTKSFILVRKADMADIPGHVFTR

SEQ ID NO: 3 - protein, 21 aa, synthetic construct
GMIIAVLILVAVVCLVTVCVI

SEQ ID NO: 4 - protein, 191 aa, synthetic construct
YRVDLVLFYRHLTRRDETLTDGKTYDAFVSYLKECRPENGEEHTFAVEILPRVLEKHFGYKLCIFERDVVPGGAVVDEIHSLIEKSRRLIIVLSKSYMSNEVRYELESGLHEALVERKIKIILIEFTPVTDFTFLPQSLKLLKSHRVLKWKADKSLSYNSRFWKNLLYLMPAKTVKPGRDEPEVLPVLSES

SEQ ID NO: 5 - protein, 541 aa, synthetic construct
MNCRELPLTLWVLISVSTAESCTSRPHITVVEGEPFYLKHCSCSLAHEIETTTKSWYKSSGSQEHVELNPRSSSRIALHDCVLEFWPVELNDTGSYFFQMKNYTQKWKLNVIRRNKHSCFTERQVTSKIVEVKKFFQITCENSYYQTLVNSTSLYKNCKKLLLENNKNPTIKKNAEFEDQGYYSCVHFLHHNGKLFNITKTFNITIVEDRSNIVPVLLGPKLNHVAVELGKNVRLNCSALLNEEDVIYWMFGEENGSDPNIHEEKEMRIMTPEGKWHASKVLRIENIGESNLNVLYNCTVASTGGTDTKSFILVRKADMADIPGHVFTRGMIIAVLILVAVVCLVTVCVIYRVDLVLFYRHLTRRDETLTDGKTYDAFVSYLKECRPENGEEHTFAVEILPRVLEKHFGYKLCIFERDVVPGGAVVDEIHSLIEKSRRLIIVLSKSYMSNEVRYELESGLHEALVERKIKIILIEFTPVTDFTFLPQSLKLLKSHRVLKWKADKSLSYNSRFWKNLLYLMPAKTVKPGRDEPEVLPVLSES

SEQ ID NO: 6 - DNA, 1623 bp, synthetic construct
  source 1..1623: mol_type = other DNA
atgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtct

SEQ ID NO: 7 - protein, 19 aa, synthetic construct
MLCLGWIFLWLVAGERIKG

SEQ ID NO: 8 - protein, 337 aa, synthetic construct
FNISGCSTKKLLWTYSTRSEEEFVLFCDLPEPQKSHFCHRNRLSPKQVPEHLPFMGSNDLSDVQWYQQPSNGDPLEDIRKSYPHIIQDKCTLHFLTPGVNNSGSYICRPKMIKSPYDVACCVKMILEVKPQTNASCEYSASHKQDLLLGSTGSISCPSLSCQSDAQSPAVTWYKNGKLLSVERSNRIVVDEVYDYHQGTYVCDYTQSDTVSSWTVRAVVQVRTIVGDTKLKPDILDPVEDTLEVELGKPLTISCKARFGFERVFNPVIKWYIKDSDLEWEVSVPEAKSIKSTLKDEIIERNIILEKVTQRDLRRKFVCFVQNSIGNTTQSVQLKEKR

SEQ ID NO: 9 - protein, 21 aa, synthetic construct
GVVLLYILLGTIGTLVAVLAA

SEQ ID NO: 10 - protein, 222 aa, synthetic construct
SALLYRHWIEIVLLYRTYQSKDQTLGDKKDFDAFVSYAKWSSFPSEATSSLSEEHLALSLFPDVLENKYGYSLCLLERDVAPGGVYAEDIVSIIKRSRRGIFILSPNYVNGPSIFELQAAVNLALDDQTLKLILIKFCYFQEPESLPHLVKKALRVLPTVTWRGLKSVPPNSRFWAKMRYHMPVKNSQGFTWNQLRITSRIFQWKGLSRTETTGRSSQPKEW

SEQ ID NO: 11 - protein, 599 aa, synthetic construct
MLCLGWIFLWLVAGERIKGFNISGCSTKKLLWTYSTRSEEEFVLFCDLPEPQKSHFCHRNRLSPKQVPEHLPFMGSNDLSDVQWYQQPSNGDPLEDIRKSYPHIIQDKCTLHFLTPGVNNSGSYICRPKMIKSPYDVACCVKMILEVKPQTNASCEYSASHKQDLLLGSTGSISCPSLSCQSDAQSPAVTWYKNGKLLSVERSNRIVVDEVYDYHQGTYVCDYTQSDTVSSWTVRAVVQVRTIVGDTKLKPDILDPVEDTLEVELGKPLTISCKARFGFERVFNPVIKWYIKDSDLEWEVSVPEAKSIKSTLKDEIIERNIILEKVTQRDLRRKFVCFVQNSIGNTTQSVQLKEKRGVVLLYILLGTIGTLVAVLAASALLYRHWIEIVLLYRTYQSKDQTLGDKKDFDAFVSYAKWSSFPSEATSSLSEEHLALSLFPDVLENKYGYSLCLLERDVAPGGVYAEDIVSIIKRSRRGIFILSPNYVNGPSIFELQAAVNLALDDQTLKLILIKFCYFQEPESLPHLVKKALRVLPTVTWRGLKSVPPNSRFWAKMRYHMPVKNSQGFTWNQLRITSRIFQWKGLSRTETTGRSSQPKEW

SEQ ID NO: 12 - DNA, 1797 bp, synthetic construct
  source 1..1797: mol_type = other DNA
atgctctgtttgggctggatatttctttggcttgttgcaggagagcgaattaaaggatttaatatttcaggttgttccacaaaaaaactcctttggacatattctacaaggagtgaagaggaatttgtcttattttgtgatttaccagagccacagaaatcacatttctgccacagaaatcgactctcaccaaaacaagtccctgagcacctgcccttcatgggtagtaacgacctatctgatgtccaatggtaccaacaaccttcgaatggagatccattagaggacattaggaaaagctatcctcacatcattcaggacaaatgtacccttcactttttgaccccaggggtgaataattctgggtcatatatttgtagacccaagatgattaagagcccctatgatgtagcctgttgtgtcaagatgattttagaagttaagccccagacaaatgcatcctgtgagtattccgcatcacataagcaagacctacttcttgggagcactggctctatttcttgccccagtctcagctgccaaagtgatgcacaaagtccagcggtaacctggtacaagaatggaaaactcctctctgtggaaaggagcaaccgaatcgtagtggatgaagtttatgactatcaccagggcacatatgtatgtgattacactcagtcggatactgtgagttcgtggacagtcagagctgttgttcaagtgagaaccattgtgggagacactaaactcaaaccagatattctggatcctgtcgaggacacactggaagtagaacttggaaagcctttaactattagctgcaaagcacgatttggctttgaaagggtctttaaccctgtcataaaatggtacatcaaagattctgacctagagtgggaagtctcagtacctgaggcgaaaagtattaaatccactttaaaggatgaaatcattgagcgtaatatcatcttggaaaaagtcactcagcgtgatcttcgcaggaagtttgtttgctttgtccagaactccattggaaacacaacccagtccgtccaactgaaagaaaagagaggagtggtgctcctgtacatcctgcttggcaccatcgggaccctggtggccgtgctggcggcgagtgccctcctctacaggcactggattgaaatagtgctgctgtaccggacctaccagagcaaggatcagacgcttggggataaaaaggattttgatgctttcgtatcctatgcaaaatggagctcttttccaagtgaggccacttcatctctgagtgaagaacacttggccctgagcctatttcctgatgttttagaaaacaaatatggatatagcctgtgtttgcttgaaagagatgtggctccaggaggagtgtatgcagaagacattgtgagcattattaagagaagcagaagaggaatatttatcttgagccccaactatgtcaatggacccagtatctttgaactacaagcagcagtgaatcttgccttggatgatcaaacactgaaactcattttaattaagttctgttacttccaagagccagagtctctacctcatctcgtgaaaaaagctctcagggttttgcccacagttacttggagaggcttaaaatcagttcctcccaattctaggttctgggccaaaatgcgctaccacatgcctgtgaaaaactctcagggattcacgtggaaccagctcagaattacctctaggatttttcagtggaaaggactcagtagaacagaaaccactgggaggagctcccagcctaaggaatgg

SEQ ID NO: 13 - DNA, 27 bp, synthetic construct
  source 1..27: mol_type = other DNA
ggcttcattttttccatttactgcaga

SEQ ID NO: 14 - DNA, 81 bp, synthetic construct
  source 1..81: mol_type = other DNA
ggcttcattttttccatttactgcagaggcttcattttttccatttactgcagaggcttcattttttccatttactgcaga

SEQ ID NO: 15 - DNA, 162 bp, synthetic construct
  source 1..162: mol_type = other DNA
ggcttcattttttccatttactgcagaggcttcattttttccatttactgcagaggcttcattttttccatttactgcagaggcttcattttttccatttactgcagaggcttcattttttccatttactgcagaggcttcattttttccatttactgcaga

SEQ ID NO: 16 - DNA, 118 bp, synthetic construct
  source 1..118: mol_type = other DNA
taggcgtgtacggtgggaggcctatataagcagagctcgtttagtgaaccgtcagatcgcctggagacgccatccacgctgttttgacctccatagaagacaccgggaccgatccagc

SEQ ID NO: 17 - DNA, 205 bp, synthetic construct
  source 1..205: mol_type = other DNA
ggcttcattttttccatttactgcagaggcttcattttttccatttactgcagaggcttcattttttccatttactgcagaactagttaggcgtgtacggtgggaggcctatataagcagagctcgtttagtgaaccgtcagatcgcctggagacgccatccacgctgttttgacctccatagaagacaccgggaccgatccagc

SEQ ID NO: 18 - DNA, 286 bp, synthetic construct
  source 1..286: mol_type = other DNA
ggcttcattttttccatttactgcagaggcttcattttttccatttactgcagaggcttcattttttccatttactgcagaggcttcattttttccatttactgcagaggcttcattttttccatttactgcagaggcttcattttttccatttactgcagaactagttaggcgtgtacggtgggaggcctatataagcagagctcgtttagtgaaccgtcagatcgcctggagacgccatccacgctgttttgacctccatagaagacaccgggaccgatccagc

SEQ ID NO: 19 - DNA, 30 bp, synthetic construct
  source 1..30: mol_type = other DNA
ggaggaaaaactgtttcatacagaaggcgt

SEQ ID NO: 20 - DNA, 90 bp, synthetic construct
  source 1..90: mol_type = other DNA
ggaggaaaaactgtttcatacagaaggcgtggaggaaaaactgtttcatacagaaggcgtggaggaaaaactgtttcatacagaaggcgt

SEQ ID NO: 21 - DNA, 186 bp, synthetic construct
  source 1..186: mol_type = other DNA
ggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtactagt

SEQ ID NO: 22 - DNA, 214 bp, synthetic construct
  source 1..214: mol_type = other DNA
ggaggaaaaactgtttcatacagaaggcgtggaggaaaaactgtttcatacagaaggcgtggaggaaaaactgtttcatacagaaggcgtactagttaggcgtgtacggtgggaggcctatataagcagagctcgtttagtgaaccgtcagatcgcctggagacgccatccacgctgttttgacctccatagaagacaccgggaccgatccagc

SEQ ID NO: 23 - DNA, 304 bp, synthetic construct
  source 1..304: mol_type = other DNA
ggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtactagttaggcgtgtacggtgggaggcctatataagcagagctcgtttagtgaaccgtcagatcgcctggagacgccatccacgctgttttgacctccatagaagacaccgggaccgatccagc

SEQ ID NO: 24 - DNA, 63 bp, synthetic construct
  source 1..63: mol_type = other DNA
ggaagcggtgagggaagaggtagcctgctcacgtgcggtgatgtggaggagaaccctgggcct

SEQ ID NO: 25 - protein, 21 aa, synthetic construct
GSGEGRGSLLTCGDVEENPGP

SEQ ID NO: 26 - DNA, 1940 bp, synthetic construct
  source 1..1940: mol_type = other DNA
ggcttcattttttccatttactgcagaggcttcattttttccatttactgcagaggcttcattttttccatttactgcagaggcttcattttttccatttactgcagaggcttcattttttccatttactgcagaggcttcattttttccatttactgcagaactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttgataa

SEQ ID NO: 27 - DNA, 1859 bp, synthetic construct
  source 1..1859: mol_type = other DNA
ggcttcattttttccatttactgcagaggcttcattttttccatttactgcagaggcttcattttttccatttactgcagaactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttgataa

SEQ ID NO: 28 - DNA, 1958 bp, synthetic construct
  source 1..1958: mol_type = other DNA
ggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttgataa

SEQ ID NO: 29 - DNA, 1868 bp, synthetic construct
  source 1..1868: mol_type = other DNA
ggaggaaaaactgtttcatacagaaggcgtggaggaaaaactgtttcatacagaaggcgtggaggaaaaactgtttcatacagaaggcgtactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttgataa

SEQ ID NO: 30 - DNA, 3818 bp, synthetic construct
  source 1..3818: mol_type = other DNA
ggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtctggaagcggtgagggaagaggtagcctgctcacgtgcggtgatgtggaggagaaccctgggcctatgctctgtttgggctggatatttctttggcttgttgcaggagagcgaattaaaggatttaatatttcaggttgttccacaaaaaaactcctttggacatattctacaaggagtgaagaggaatttgtcttattttgtgatttaccagagccacagaaatcacatttctgccacagaaatcgactctcaccaaaacaagtccctgagcacctgcccttcatgggtagtaacgacctatctgatgtccaatggtaccaacaaccttcgaatggagatccattagaggacattaggaaaagctatcctcacatcattcaggacaaatgtacccttcactttttgaccccaggggtgaataattctgggtcatatatttgtagacccaagatgattaagagcccctatgatgtagcctgttgtgtcaagatgattttagaagttaagccccagacaaatgcatcctgtgagtattccgcatcacataagcaagacctacttcttgggagcactggctctatttcttgccccagtctcagctgccaaagtgatgcacaaagtccagcggtaacctggtacaagaatggaaaactcctctctgtggaaaggagcaaccgaatcgtagtggatgaagtttatgactatcaccagggcacatatgtatgtgattacactcagtcggatactgtgagttcgtggacagtcagagctgttgttcaagtgagaaccattgtgggagacactaaactcaaaccagatattctggatcctgtcgaggacacactggaagtagaacttggaaagcctttaactattagctgcaaagcacgatttggctttgaaagggtctttaaccctgtcataaaatggtacatcaaagattctgacctagagtgggaagtctcagtacctgaggcgaaaagtattaaatccactttaaaggatgaaatcattgagcgtaatatcatcttggaaaaagtcactcagcgtgatcttcgcaggaagtttgtttgctttgtccagaactccattggaaacacaacccagtccgtccaactgaaagaaaagagaggagtggtgctcctgtacatcctgcttggcaccatcgggaccctggtggccgtgctggcggcgagtgccctcctctacaggcactggattgaaatagtgctgctgtaccggacctaccagagcaaggatcagacgcttggggataaaaaggattttgatgctttcgtatcctatgcaaaatggagctcttttccaagtgaggccacttcatctctgagtgaagaacacttggccctgagcctatttcctgatgttttagaaaacaaatatggatatagcctgtgtttgcttgaaagagatgtggctccaggaggagtgtatgcagaagacattgtgagcattattaagagaagcagaagaggaatatttatcttgagccccaactatgtcaatggacccagtatctttgaactacaagcagcagtgaatcttgccttggatgatcaaacactgaaactcattttaattaagttctgttacttccaagagccagagtctctacctcatctcgtgaaaaaagctctcagggttttgcccacagttacttggagaggcttaaaatcagttcctcccaattctaggttctgggccaaaatgcgctaccacatgcctgtgaaaaactctcagggattcacgtggaaccagctcagaattacctctaggatttttcagtggaaaggactcagtagaacagaaaccactgggaggagctcccagcctaaggaatggtgataa

SEQ ID NO: 31 - DNA, 3719 bp, synthetic construct
  source 1..3719: mol_type = other DNA
ggcttcattttttccatttactgcagaggcttcattttttccatttactgcagaggcttcattttttccatttactgcagaactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtctggaagcggtgagggaagaggtagcctgctcacgtgcggtgatgtggaggagaaccctgggcctatgctctgtttgggctggatatttctttggcttgttgcaggagagcgaattaaaggatttaatatttcaggttgttccacaaaaaaactcctttggacatattctacaaggagtgaagaggaatttgtcttattttgtgatttaccagagccacagaaatcacatttctgccacagaaatcgactctcaccaaaacaagtccctgagcacctgcccttcatgggtagtaacgacctatctgatgtccaatggtaccaacaaccttcgaatggagatccattagaggacattaggaaaagctatcctcacatcattcaggacaaatgtacccttcactttttgaccccaggggtgaataattctgggtcatatatttgtagacccaagatgattaagagcccctatgatgtagcctgttgtgtcaagatgattttagaagttaagccccagacaaatgcatcctgtgagtattccgcatcacataagcaagacctacttcttgggagcactggctctatttcttgccccagtctcagctgccaaagtgatgcacaaagtccagcggtaacctggtacaagaatggaaaactcctctctgtggaaaggagcaaccgaatcgtagtggatgaagtttatgactatcaccagggcacatatgtatgtgattacactcagtcggatactgtgagttcgtggacagtcagagctgttgttcaagtgagaaccattgtgggagacactaaactcaaaccagatattctggatcctgtcgaggacacactggaagtagaacttggaaagcctttaactattagctgcaaagcacgatttggctttgaaagggtctttaaccctgtcataaaatggtacatcaaagattctgacctagagtgggaagtctcagtacctgaggcgaaaagtattaaatccactttaaaggatgaaatcattgagcgtaatatcatcttggaaaaagtcactcagcgtgatcttcgcaggaagtttgtttgctttgtccagaactccattggaaacacaacccagtccgtccaactgaaagaaaagagaggagtggtgctcctgtacatcctgcttggcaccatcgggaccctggtggccgtgctggcggcgagtgccctcctctacaggcactggattgaaatagtgctgctgtaccggacctaccagagcaaggatcagacgcttggggataaaaaggattttgatgctttcgtatcctatgcaaaatggagctcttttccaagtgaggccacttcatctctgagtgaagaacacttggccctgagcctatttcctgatgttttagaaaacaaatatggatatagcctgtgtttgcttgaaagagatgtggctccaggaggagtgtatgcagaagacattgtgagcattattaagagaagcagaagaggaatatttatcttgagccccaactatgtcaatggacccagtatctttgaactacaagcagcagtgaatcttgccttggatgatcaaacactgaaactcattttaattaagttctgttacttccaagagccagagtctctacctcatctcgtgaaaaaagctctcagggttttgcccacagttacttggagaggcttaaaatcagttcctcccaattctaggttctgggccaaaatgcgctaccacatgcctgtgaaaaactctcagggattcacgtggaaccagctcagaattacctctaggatttttcagtggaaaggactcagtagaacagaaaccactgggaggagctcccagcctaaggaatggtgataa

SEQ ID NO: 32 - DNA, 3818 bp, synthetic construct; identical to SEQ ID NO: 30
  source 1..3818: mol_type = other DNA

SEQ ID NO: 33 - DNA, 3728 bp, synthetic construct
  source 1..3728: mol_type = other DNA
ggaggaaaaactgtttcatacagaaggcgtggaggaaaaactgtttcatacagaaggcgtggaggaaaaactgtttcatacagaaggcgtactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtctggaagcggtgagggaagaggtagcctgctcacgtgcggtgatgtggaggagaaccctgggcctatgctctgtttgggctggatatttctttggcttgttgcaggagagcgaattaaaggatttaatatttcaggttgttccacaaaaaaactcctttggacatattctacaaggagtgaagaggaatttgtcttattttgtgatttaccagagccacagaaatcacatttctgccacagaaatcgactctcaccaaaacaagtccctgagcacctgcccttcatgggtagtaacgacctatctgatgtccaatggtaccaacaaccttcgaatggagatccattagaggacattaggaaaagctatcctcacatcattcaggacaaatgtacccttcactttttgaccccaggggtgaataattctgggtcatatatttgtagacccaagatgattaagagcccctatgatgtagcctgttgtgtcaagatgattttagaagttaagccccagacaaatgcatcctgtgagtattccgcatcacataagcaagacctacttcttgggagcactggctctatttcttgccccagtctcagctgccaaagtgatgcacaaagtccagcggtaacctggtacaagaatggaaaactcctctctgtggaaaggagcaaccgaatcgtagtggatgaagtttatgactatcaccagggcacatatgtatgtgattacactcagtcggatactgtgagttcgtggacagtcagagctgttgttcaagtgagaaccattgtgggagacactaaactcaaaccagatattctggatcctgtcgaggacacactggaagtagaacttggaaagcctttaactattagctgcaaagcacgatttggctttgaaagggtctttaaccctgtcataaaatggtacatcaaagattctgacctagagtgggaagtctcagtacctgaggcgaaaagtattaaatccactttaaaggatgaaatcattgagcgtaatatcatcttggaaaaagtcactcagcgtgatcttcgcaggaagtttgtttgctttgtccagaactccattggaaacacaacccagtccgtccaactgaaagaaaagagaggagtggtgctcctgtacatcctgcttggcaccatcgggaccctggtggccgtgctggcggcgagtgccctcctctacaggcactggattgaaatagtgctgctgtaccggacctaccagagcaaggatcagacgcttggggataaaaaggattttgatgctttcgtatcctatgcaaaatggagctcttttccaagtgaggccacttcatctctgagtgaagaacacttggccctgagcctatttcctgatgttttagaaaacaaatatggatatagcctgtgtttgcttgaaagagatgtggctccaggaggagtgtatgcagaagacattgtgagcattattaagagaagcagaagaggaatatttatcttgagccccaactatgtcaatggacccagtatctttgaactacaagcagcagtgaatcttgccttggatgatcaaacactgaaactcattttaattaagttctgttacttccaagagccagagtctctacctcatctcgtgaaaaaagctctcagggttttgcccacagttacttggagaggcttaaaatcagttcctcccaattctaggttctgggccaaaatgcgctaccacatgcctgtgaaaaactctcagggattcacgtggaaccagctcagaattacctctaggatttttcagtggaaaggactcagtagaacagaaaccactgggaggagctcccagcctaaggaatggtgataa

SEQ ID NO: 34 - protein, 21 aa, synthetic construct
MALPVTALLLPLALLLHAARP

SEQ ID NO: 35 - protein, 45 aa, synthetic construct
GTINVHDVETQFNQYKTEAASRYNLTISDVSVSDVPFPFSAQSGA

SEQ ID NO: 36 - protein, 35 aa, synthetic construct
QFNQYKTEAASRYNLTISDVSVSDVPFPFSAQSGA

SEQ ID NO: 37 - protein, 5 aa, synthetic construct
GYAMS

SEQ ID NO: 38 - protein, 17 aa, synthetic construct
TISSGGTYIYYPDSVKG

SEQ ID NO: 39 - protein, 12 aa, synthetic construct
LGGDNYYEYFDV

SEQ ID NO: 40 - protein, 15 aa, synthetic construct
RASKSVSTSGYSYMH

SEQ ID NO: 41 - protein, 7 aa, synthetic construct
LASNLES

SEQ ID NO: 42 - protein, 9 aa, synthetic construct
QHSRELPFT

SEQ ID NO: 43 - protein, 121 aa, synthetic construct
EVQLVESGGGLVKPGGSLRLSCAASGFTFSGYAMSWVRQAPGKGLEWVSTISSGGTYIYYPDSVKGRFTISRDNAKNSLYLQMNSLRAEDTAVYYCARLGGDNYYEYFDVWGKGTTVTVSS

SEQ ID NO: 44 - protein, 113 aa, synthetic construct
DIVLTQSPASLAVSPGQRATITCRASKSVSTSGYSYMHWYQQKPGQPPKLLIYLASNLESGVPARFSGSGSGTDFTLTINPVEANDTANYYCQHSRELPFTFGGGTKVEIKRT

SEQ ID NO: 45 - protein, 249 aa, synthetic construct
EVQLVESGGGLVKPGGSLRLSCAASGFTFSGYAMSWVRQAPGKGLEWVSTISSGGTYIYYPDSVKGRFTISRDNAKNSLYLQMNSLRAEDTAVYYCARLGGDNYYEYFDVWGKGTTVTVSSGGGGSGGGGSGGGGSDIVLTQSPASLAVSPGQRATITCRASKSVSTSGYSYMHWYQQKPGQPPKLLIYLASNLESGVPARFSGSGSGTDFTLTINPVEANDTANYYCQHSRELPFTFGGGTKVEIKRT

SEQ ID NO: 46 - protein, 5 aa, synthetic construct
RYGMS

SEQ ID NO: 47 - protein, 17 aa, synthetic construct
TISGGGTYIYYPDSVKG

SEQ ID NO: 48 - protein, 13 aa, synthetic construct
DNYGRNYDYGMDY

SEQ ID NO: 49 - protein, 10 aa, synthetic construct
SATSSVSYIH

SEQ ID NO: 50 - protein, 7 aa, synthetic construct
STSNLAS

SEQ ID NO: 51 - protein, 9 aa, synthetic construct
QQRSSSPFT

SEQ ID NO: 52 - protein, 122 aa, synthetic construct
EVQLVESGGGLVKPGGSLRLSCAASGFTFSRYGMSWVRQAPGKRLEWVSTISGGGTYIYYPDSVKGRFTISRDNAKNTLYLQMNSLRAEDTAVYYCTRDNYGRNYDYGMDYWGQGTLVTVSS

SEQ ID NO: 53 - protein, 106 aa, synthetic construct
EIVLTQSPATLSLSPGERATLTCSATSSVSYIHWYQQRPGQSPRLLIYSTSNLASGIPARFSGSGSGSDYTLTISSLEPEDFAVYYCQQRSSSPFTFGSGTKVEIK

SEQ ID NO: 54 - protein, 243 aa, synthetic construct
EVQLVESGGGLVKPGGSLRLSCAASGFTFSRYGMSWVRQAPGKRLEWVSTISGGGTYIYYPDSVKGRFTISRDNAKNTLYLQMNSLRAEDTAVYYCTRDNYGRNYDYGMDYWGQGTLVTVSSGGGGSGGGGSGGGGSEIVLTQSPATLSLSPGERATLTCSATSSVSYIHWYQQRPGQSPRLLIYSTSNLASGIPARFSGSGSGSDYTLTISSLEPEDFAVYYCQQRSSSPFTFGSGTKVEIK

SEQ ID NO: 55 - protein, 5 aa, synthetic construct
TYAMS

SEQ ID NO: 56 - protein, 16 aa, synthetic construct
SIGRAGSTYYSDSVKG

SEQ ID NO: 57 - protein, 12 aa, synthetic construct
GPIYNDYDEFAY

SEQ ID NO: 58 - protein, 17 aa, synthetic construct
KSSQSVLYSSNQKNYLA

SEQ ID NO: 59 - protein, 7 aa, synthetic construct
WASTRES

SEQ ID NO: 60 - protein, 8 aa, synthetic construct
HQYLSSLT

SEQ ID NO: 61 - protein, 120 aa, synthetic construct
QVQLVESGGGLVKPGGSLRLSCAASGFTFSTYAMSWIRQAPGKGLEWVASIGRAGSTYYSDSVKGRFTISRDNAKNSLYLQMNSLRAEDTAVYYCARGPIYNDYDEFAYWGQGTLVTVSS

SEQ ID NO: 62 - protein, 113 aa, synthetic construct
EIVLTQSPATLSLSPGERATLSCKSSQSVLYSSNQKNYLAWYQQKPGQAPRLLIYWASTRESGIPARFSGSGSGTDFTLTISSLEPEDFAVYYCHQYLSSLTFGGGTKVEIKR

SEQ ID NO: 63 - protein, 248 aa, synthetic construct
QVQLVESGGGLVKPGGSLRLSCAASGFTFSTYAMSWIRQAPGKGLEWVASIGRAGSTYYSDSVKGRFTISRDNAKNSLYLQMNSLRAEDTAVYYCARGPIYNDYDEFAYWGQGTLVTVSSGGGGSGGGGSGGGGSEIVLTQSPATLSLSPGERATLSCKSSQSVLYSSNQKNYLAWYQQKPGQAPRLLIYWASTRESGIPARFSGSGSGTDFTLTISSLEPEDFAVYYCHQYLSSLTFGGGTKVEIKR

SEQ ID NO: 64 - protein, 45 aa, synthetic construct
TTTPAPRPPTPAPTIASQPLSLRPEACRPAAGGAVHTRGLDFACD

SEQ ID NO: 65 - protein, 15 aa, synthetic construct
KHLCPSPLFPGPSKP

SEQ ID NO: 66 - protein, 24 aa, synthetic construct
IYIWAPLAGTCGVLLLSLVITLYC

SEQ ID NO: 67 - protein, 27 aa, synthetic construct
FWVLVVVGGVLACYSLLVTVAFIIFWV

SEQ ID NO: 68 - protein, 42 aa, synthetic construct
KRGRKKLLYIFKQPFMRPVQTTQEEDGCSCRFPEEEEGGCEL

SEQ ID NO: 69 - protein, 41 aa, synthetic construct
RSKRSRLLHSDYMNMTPRRPGPTRKHYQPYAPPRDFAAYRS

SEQ ID NO: 70 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 71 - protein, 112 aa, synthetic construct
RVKFSRSADAPAYKQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 72 - protein, 493 aa, synthetic construct
MALPVTALLLPLALLLHAARPEVQLVESGGGLVKPGGSLRLSCAASGFTFSGYAMSWVRQAPGKGLEWVSTISSGGTYIYYPDSVKGRFTISRDNAKNSLYLQMNSLRAEDTAVYYCARLGGDNYYEYFDVWGKGTTVTVSSGGGGSGGGGSGGGGSDIVLTQSPASLAVSPGQRATITCRASKSVSTSGYSYMHWYQQKPGQPPKLLIYLASNLESGVPARFSGSGSGTDFTLTINPVEANDTANYYCQHSRELPFTFGGGTKVEIKRTTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 73 - protein, 487 aa, synthetic construct
MALPVTALLLPLALLLHAARPEVQLVESGGGLVKPGGSLRLSCAASGFTFSRYGMSWVRQAPGKRLEWVSTISGGGTYIYYPDSVKGRFTISRDNAKNTLYLQMNSLRAEDTAVYYCTRDNYGRNYDYGMDYWGQGTLVTVSSGGGGSGGGGSGGGGSEIVLTQSPATLSLSPGERATLTCSATSSVSYIHWYQQRPGQSPRLLIYSTSNLASGIPARFSGSGSGSDYTLTISSLEPEDFAVYYCQQRSSSPFTFGSGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 74 - protein, 492 aa, synthetic construct
MALPVTALLLPLALLLHAARPQVQLVESGGGLVKPGGSLRLSCAASGFTFSTYAMSWIRQAPGKGLEWVASIGRAGSTYYSDSVKGRFTISRDNAKNSLYLQMNSLRAEDTAVYYCARGPIYNDYDEFAYWGQGTLVTVSSGGGGSGGGGSGGGGSEIVLTQSPATLSLSPGERATLSCKSSQSVLYSSNQKNYLAWYQQKPGQAPRLLIYWASTRESGIPARFSGSGSGTDFTLTISSLEPEDFAVYYCHQYLSSLTFGGGTKVEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 75 - protein, 465 aa, synthetic construct
MALPVTALLLPLALLLHAARPEVQLVESGGGLVKPGGSLRLSCAASGFTFSGYAMSWVRQAPGKGLEWVSTISSGGTYIYYPDSVKGRFTISRDNAKNSLYLQMNSLRAEDTAVYYCARLGGDNYYEYFDVWGKGTTVTVSSGGGGSGGGGSGGGGSDIVLTQSPASLAVSPGQRATITCRASKSVSTSGYSYMHWYQQKPGQPPKLLIYLASNLESGVPARFSGSGSGTDFTLTINPVEANDTANYYCQHSRELPFTFGGGTKVEIKRTKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 76 - protein, 459 aa, synthetic construct
MALPVTALLLPLALLLHAARPEVQLVESGGGLVKPGGSLRLSCAASGFTFSRYGMSWVRQAPGKRLEWVSTISGGGTYIYYPDSVKGRFTISRDNAKNTLYLQMNSLRAEDTAVYYCTRDNYGRNYDYGMDYWGQGTLVTVSSGGGGSGGGGSGGGGSEIVLTQSPATLSLSPGERATLTCSATSSVSYIHWYQQRPGQSPRLLIYSTSNLASGIPARFSGSGSGSDYTLTISSLEPEDFAVYYCQQRSSSPFTFGSGTKVEIKKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 77 - protein, 464 aa, synthetic construct
MALPVTALLLPLALLLHAARPQVQLVESGGGLVKPGGSLRLSCAASGFTFSTYAMSWIRQAPGKGLEWVASIGRAGSTYYSDSVKGRFTISRDNAKNSLYLQMNSLRAEDTAVYYCARGPIYNDYDEFAYWGQGTLVTVSSGGGGSGGGGSGGGGSEIVLTQSPATLSLSPGERATLSCKSSQSVLYSSNQKNYLAWYQQKPGQAPRLLIYWASTRESGIPARFSGSGSGTDFTLTISSLEPEDFAVYYCHQYLSSLTFGGGTKVEIKRKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 78 - protein, 493 aa, synthetic construct
MALPVTALLLPLALLLHAARPEVQLVESGGGLVKPGGSLRLSCAASGFTFSGYAMSWVRQAPGKGLEWVSTISSGGTYIYYPDSVKGRFTISRDNAKNSLYLQMNSLRAEDTAVYYCARLGGDNYYEYFDVWGKGTTVTVSSGGGGSGGGGSGGGGSDIVLTQSPASLAVSPGQRATITCRASKSVSTSGYSYMHWYQQKPGQPPKLLIYLASNLESGVPARFSGSGSGTDFTLTINPVEANDTANYYCQHSRELPFTFGGGTKVEIKRTTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 79 - protein, 487 aa, synthetic construct
MALPVTALLLPLALLLHAARPEVQLVESGGGLVKPGGSLRLSCAASGFTFSRYGMSWVRQAPGKRLEWVSTISGGGTYIYYPDSVKGRFTISRDNAKNTLYLQMNSLRAEDTAVYYCTRDNYGRNYDYGMDYWGQGTLVTVSSGGGGSGGGGSGGGGSEIVLTQSPATLSLSPGERATLTCSATSSVSYIHWYQQRPGQSPRLLIYSTSNLASGIPARFSGSGSGSDYTLTISSLEPEDFAVYYCQQRSSSPFTFGSGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 80 - protein, 492 aa, synthetic construct
MALPVTALLLPLALLLHAARPQVQLVESGGGLVKPGGSLRLSCAASGFTFSTYAMSWIRQAPGKGLEWVASIGRAGSTYYSDSVKGRFTISRDNAKNSLYLQMNSLRAEDTAVYYCARGPIYNDYDEFAYWGQGTLVTVSSGGGGSGGGGSGGGGSEIVLTQSPATLSLSPGERATLSCKSSQSVLYSSNQKNYLAWYQQKPGQAPRLLIYWASTRESGIPARFSGSGSGTDFTLTISSLEPEDFAVYYCHQYLSSLTFGGGTKVEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 81 - protein, 465 aa, synthetic construct
MALPVTALLLPLALLLHAARPEVQLVESGGGLVKPGGSLRLSCAASGFTFSGYAMSWVRQAPGKGLEWVSTISSGGTYIYYPDSVKGRFTISRDNAKNSLYLQMNSLRAEDTAVYYCARLGGDNYYEYFDVWGKGTTVTVSSGGGGSGGGGSGGGGSDIVLTQSPASLAVSPGQRATITCRASKSVSTSGYSYMHWYQQKPGQPPKLLIYLASNLESGVPARFSGSGSGTDFTLTINPVEANDTANYYCQHSRELPFTFGGGTKVEIKRTKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 82 - protein, 459 aa, synthetic construct
MALPVTALLLPLALLLHAARPEVQLVESGGGLVKPGGSLRLSCAASGFTFSRYGMSWVRQAPGKRLEWVSTISGGGTYIYYPDSVKGRFTISRDNAKNTLYLQMNSLRAEDTAVYYCTRDNYGRNYDYGMDYWGQGTLVTVSSGGGGSGGGGSGGGGSEIVLTQSPATLSLSPGERATLTCSATSSVSYIHWYQQRPGQSPRLLIYSTSNLASGIPARFSGSGSGSDYTLTISSLEPEDFAVYYCQQRSSSPFTFGSGTKVEIKKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 83 - protein, 464 aa, synthetic construct
MALPVTALLLPLALLLHAARPQVQLVESGGGLVKPGGSLRLSCAASGFTFSTYAMSWIRQAPGKGLEWVASIGRAGSTYYSDSVKGRFTISRDNAKNSLYLQMNSLRAEDTAVYYCARGPIYNDYDEFAYWGQGTLVTVSSGGGGSGGGGSGGGGSEIVLTQSPATLSLSPGERATLSCKSSQSVLYSSNQKNYLAWYQQKPGQAPRLLIYWASTRESGIPARFSGSGSGTDFTLTISSLEPEDFAVYYCHQYLSSLTFGGGTKVEIKRKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 84 - DNA, 1479 bp, synthetic construct
  source 1..1479: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtggctatgccatgagctgggtccgccaggctccagggaaggggctggagtgggtctcaaccattagtagtggcggaacctacatatactaccccgactcagtgaagggccgattcaccatctccagagacaacgccaagaactcactgtatctgcaaatgaacagcctgagagccgaggacacggccgtgtattactgtgcgagacttgggggggataattactacgaatacttcgatgtctggggcaaagggaccacggtcaccgtctcctccggcggtggcggatccggcggtggcggatccggcggtggcggatccgacattgtgctgacccagtctccagcctccttggccgtgtctccaggacagagggccaccatcacctgcagagccagtaagagtgtcagtaccagcggatactcctacatgcactggtatcagcagaaaccaggacaacctcctaaactcctgatttacctggcatccaatctggagagcggggtcccagccaggttcagcggcagtgggtctgggaccgatttcaccctcacaattaatcctgtggaagctaatgatactgcaaattattactgtcagcacagtagggagctgcctttcacattcggcggagggaccaaggtggagatcaaacgaactacaacaacccctgcccccagacctcctaccccagcccctacaattgccagccagcctctgagcctgaggcccgaggcttgtagacctgctgctggcggagccgtgcacaccagaggactggatttcgcctgcgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 85 - DNA, 1461 bp, synthetic construct
  source 1..1461: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtaggtatggcatgagctgggtccgccaggctccagggaagaggctggagtgggtctcaaccattagtggcggaggcacctacatatactacccagactcagtgaagggccgattcaccatctccagagacaacgccaagaacaccctgtatctgcaaatgaacagcctgagagccgaggacacggctgtgtattactgtaccagagataactatggccgcaactatgattatggcatggattattggggccagggcaccctggtgaccgtgagcagcggtggtggcggttccggcggtggcggttccggtggtggcggttccgaaattgtgttgacacagtctccagccaccctgtctttgtctccaggggaaagagccaccctcacctgcagcgccaccagcagtgttagctacatccactggtatcaacagaggcctggccagagccccaggctcctcatctatagcacctccaacctggccagcggcatcccagccaggttcagtggcagtgggtctgggagcgactacactctcaccatcagcagcctagagcctgaagattttgcagtttattactgtcagcagcgtagcagctcccctttcacctttggcagcggcaccaaagtggaaattaaaacaacaacccctgcccccagacctcctaccccagcccctacaattgccagccagcctctgagcctgaggcccgaggcttgtagacctgctgctggcggagccgtgcacaccagaggactggatttcgcctgcgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 86 - DNA, 1476 bp, synthetic construct
  source 1..1476: mol_type = other DNA
atggccttaccagtgaccgccttgctcctgccgctggccttgctgctccacgccgccaggccgcaggtgcagctggttgaatctggcggcggacttgtgaagcctggcggatctctgagactgagctgtgccgccagcggcttcacctttagcacatacgccatgagctggatcagacaggcccctggcaaaggcctggaatgggtggcgtctattggcagagccggcagcacctactacagcgactctgtgaagggcagattcaccatcagccgggacaacgccaagaacagcctgtacctgcagatgaactccctgagagccgaggacaccgccgtgtactattgtgccagaggacccatctacaacgactacgacgagttcgcctattggggccagggcacactggtcacagtttctagcggcggtggcggaagcggaggcggtggctccggtggcggaggcagcgaaattgtgctgacacagagccccgccacactgtcactttctccaggcgaaagagccacactgagctgcaagagcagccagagcgtgctgtactccagcaaccagaagaactacctggcctggtatcagcagaagcccggccaagctcctcggctgctgatctattgggccagcacaagagagagcggcatccctgccagattttctggcagcggctctggcaccgatttcaccctgaccataagcagcctggaacctgaggacttcgccgtgtattactgccaccagtacctgagcagcctgacctttggcggaggcaccaaggtggaaatcaagcggacaacaacccctgcccccagacctcctaccccagcccctacaattgccagccagcctctgagcctgaggcccgaggcttgtagacctgctgctggcggagccgtgcacaccagaggactggatttcgcctgcgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 87 - DNA, 1395 bp, synthetic construct
  source 1..1395: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtggctatgccatgagctgggtccgccaggctccagggaaggggctggagtgggtctcaaccattagtagtggcggaacctacatatactaccccgactcagtgaagggccgattcaccatctccagagacaacgccaagaactcactgtatctgcaaatgaacagcctgagagccgaggacacggccgtgtattactgtgcgagacttgggggggataattactacgaatacttcgatgtctggggcaaagggaccacggtcaccgtctcctccggcggtggcggatccggcggtggcggatccggcggtggcggatccgacattgtgctgacccagtctccagcctccttggccgtgtctccaggacagagggccaccatcacctgcagagccagtaagagtgtcagtaccagcggatactcctacatgcactggtatcagcagaaaccaggacaacctcctaaactcctgatttacctggcatccaatctggagagcggggtcccagccaggttcagcggcagtgggtctgggaccgatttcaccctcacaattaatcctgtggaagctaatgatactgcaaattattactgtcagcacagtagggagctgcctttcacattcggcggagggaccaaggtggagatcaaacgaactaagcacctgtgtccttctccactgttccccggccctagcaagcctttctgggtcctggtggtggtgggcggagtgctggcctgctacagcctgctggtgaccgtggcctttatcatcttctgggtgaggagtaagaggagcaggctcctgcacagtgactacatgaacatgactcctagaagacctgggcctaccagaaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 88 - DNA, 1377 bp, synthetic construct
  source 1..1377: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtaggtatggcatgagctgggtccgccaggctccagggaagaggctggagtgggtctcaaccattagtggcggaggcacctacatatactacccagactcagtgaagggccgattcaccatctccagagacaacgccaagaacaccctgtatctgcaaatgaacagcctgagagccgaggacacggctgtgtattactgtaccagagataactatggccgcaactatgattatggcatggattattggggccagggcaccctggtgaccgtgagcagcggtggtggcggttccggcggtggcggttccggtggtggcggttccgaaattgtgttgacacagtctccagccaccctgtctttgtctccaggggaaagagccaccctcacctgcagcgccaccagcagtgttagctacatccactggtatcaacagaggcctggccagagccccaggctcctcatctatagcacctccaacctggccagcggcatcccagccaggttcagtggcagtgggtctgggagcgactacactctcaccatcagcagcctagagcctgaagattttgcagtttattactgtcagcagcgtagcagctcccctttcacctttggcagcggcaccaaagtggaaattaaaaagcacctgtgtccttctccactgttccccggccctagcaagcctttctgggtcctggtggtggtgggcggagtgctggcctgctacagcctgctggtgaccgtggcctttatcatcttctgggtgaggagtaagaggagcaggctcctgcacagtgactacatgaacatgactcctagaagacctgggcctaccagaaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 89 - DNA, 1392 bp, synthetic construct
  source 1..1392: mol_type = other DNA
atggccttaccagtgaccgccttgctcctgccgctggccttgctgctccacgccgccaggccgcaggtgcagctggttgaatctggcggcggacttgtgaagcctggcggatctctgagactgagctgtgccgccagcggcttcacctttagcacatacgccatgagctggatcagacaggcccctggcaaaggcctggaatgggtggcgtctattggcagagccggcagcacctactacagcgactctgtgaagggcagattcaccatcagccgggacaacgccaagaacagcctgtacctgcagatgaactccctgagagccgaggacaccgccgtgtactattgtgccagaggacccatctacaacgactacgacgagttcgcctattggggccagggcacactggtcacagtttctagcggcggtggcggaagcggaggcggtggctccggtggcggaggcagcgaaattgtgctgacacagagccccgccacactgtcactttctccaggcgaaagagccacactgagctgcaagagcagccagagcgtgctgtactccagcaaccagaagaactacctggcctggtatcagcagaagcccggccaagctcctcggctgctgatctattgggccagcacaagagagagcggcatccctgccagattttctggcagcggctctggcaccgatttcaccctgaccataagcagcctggaacctgaggacttcgccgtgtattactgccaccagtacctgagcagcctgacctttggcggaggcaccaaggtggaaatcaagcggaagcacctgtgtccttctccactgttccccggccctagcaagcctttctgggtcctggtggtggtgggcggagtgctggcctgctacagcctgctggtgaccgtggcctttatcatcttctgggtgaggagtaagaggagcaggctcctgcacagtgactacatgaacatgactcctagaagacctgggcctaccagaaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 90 - DNA, 1479 bp, synthetic construct
  source 1..1479: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtggctatgccatgagctgggtccgccaggctccagggaaggggctggagtgggtctcaaccattagtagtggcggaacctacatatactaccccgactcagtgaagggccgattcaccatctccagagacaacgccaagaactcactgtatctgcaaatgaacagcctgagagccgaggacacggccgtgtattactgtgcgagacttgggggggataattactacgaatacttcgatgtctggggcaaagggaccacggtcaccgtctcctccggcggtggcggatccggcggtggcggatccggcggtggcggatccgacattgtgctgacccagtctccagcctccttggccgtgtctccaggacagagggccaccatcacctgcagagccagtaagagtgtcagtaccagcggatactcctacatgcactggtatcagcagaaaccaggacaacctcctaaactcctgatttacctggcatccaatctggagagcggggtcccagccaggttcagcggcagtgggtctgggaccgatttcaccctcacaattaatcctgtggaagctaatgatactgcaaattattactgtcagcacagtagggagctgcctttcacattcggcggagggaccaaggtggagatcaaacgaactacaacaacccctgcccccagacctcctaccccagcccctacaattgccagccagcctctgagcctgaggcccgaggcttgtagacctgctgctggcggagccgtgcacaccagaggactggatttcgcctgcgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgc

SEQ ID NO: 91 - DNA, 1461 bp, synthetic construct
  source 1..1461: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtaggtatggcatgagctgggtccgccaggctccagggaagaggctggagtgggtctcaaccattagtggcggaggcacctacatatactacccagactcagtgaagggccgattcaccatctccagagacaacgccaagaacaccctgtatctgcaaatgaacagcctgagagccgaggacacggctgtgtattactgtaccagagataactatggccgcaactatgattatggcatggattattggggccagggcaccctggtgaccgtgagcagcggtggtggcggttccggcggtggcggttccggtggtggcggttccgaaattgtgttgacacagtctccagccaccctgtctttgtctccaggggaaagagccaccctcacctgcagcgccaccagcagtgttagctacatccactggtatcaacagaggcctggccagagccccaggctcctcatctatagcacctccaacctggccagcggcatcccagccaggttcagtggcagtgggtctgggagcgactacactctcaccatcagcagcctagagcctgaagattttgcagtttattactgtcagcagcgtagcagctcccctttcacctttggcagcggcaccaaagtggaaattaaaacaacaacccctgcccccagacctcctaccccagcccctacaattgccagccagcctctgagcctgaggcccgaggcttgtagacctgctgctggcggagccgtgcacaccagaggactggatttcgcctgcgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgc

SEQ ID NO: 92 - DNA, 1476 bp, synthetic construct
  source 1..1476: mol_type = other DNA
atggccttaccagtgaccgccttgctcctgccgctggccttgctgctccacgccgccaggccgcaggtgcagctggttgaatctggcggcggacttgtgaagcctggcggatctctgagactgagctgtgccgccagcggcttcacctttagcacatacgccatgagctggatcagacaggcccctggcaaaggcctggaatgggtggcgtctattggcagagccggcagcacctactacagcgactctgtgaagggcagattcaccatcagccgggacaacgccaagaacagcctgtacctgcagatgaactccctgagagccgaggacaccgccgtgtactattgtgccagaggacccatctacaacgactacgacgagttcgcctattggggccagggcacactggtcacagtttctagcggcggtggcggaagcggaggcggtggctccggtggcggaggcagcgaaattgtgctgacacagagccccgccacactgtcactttctccaggcgaaagagccacactgagctgcaagagcagccagagcgtgctgtactccagcaaccagaagaactacctggcctggtatcagcagaagcccggccaagctcctcggctgctgatctattgggccagcacaagagagagcggcatccctgccagattttctggcagcggctctggcaccgatttcaccctgaccataagcagcctggaacctgaggacttcgccgtgtattactgccaccagtacctgagcagcctgacctttggcggaggcaccaaggtggaaatcaagcggacaacaacccctgcccccagacctcctaccccagcccctacaattgccagccagcctctgagcctgaggcccgaggcttgtagacctgctgctggcggagccgtgcacaccagaggactggatttcgcctgcgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgc

SEQ ID NO: 93 - DNA, 1395 bp, synthetic construct
  source 1..1395: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtggctatgccatgagctgggtccgccaggctccagggaaggggctggagtgggtctcaaccattagtagtggcggaacctacatatactaccccgactcagtgaagggccgattcaccatctccagagacaacgccaagaactcactgtatctgcaaatgaacagcctgagagccgaggacacggccgtgtattactgtgcgagacttgggggggataattactacgaatacttcgatgtctggggcaaagggaccacggtcaccgtctcctccggcggtggcggatccggcggtggcggatccggcggtggcggatccgacattgtgctgacccagtctccagcctccttggccgtgtctccaggacagagggccaccatcacctgcagagccagtaagagtgtcagtaccagcggatactcctacatgcactggtatcagcagaaaccaggacaacctcctaaactcctgatttacctggcatccaatctggagagcggggtcccagccaggttcagcggcagtgggtctgggaccgatttcaccctcacaattaatcctgtggaagctaatgatactgcaaattattactgtcagcacagtagggagctgcctttcacattcggcggagggaccaaggtggagatcaaacgaactaagcacctgtgtccttctccactgttccccggccctagcaagcctttctgggtcctggtggtggtgggcggagtgctggcctgctacagcctgctggtgaccgtggcctttatcatcttctgggtgaggagtaagaggagcaggctcctgcacagtgactacatgaacatgactcctagaagacctgggcctaccagaaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgc

SEQ ID NO: 94 - DNA, 1377 bp, synthetic construct
  source 1..1377: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtaggtatggcatgagctgggtccgccaggctccagggaagaggctggagtgggtctcaaccattagtggcggaggcacctacatatactacccagactcagtgaagggccgattcaccatctccagagacaacgccaagaacaccctgtatctgcaaatgaacagcctgagagccgaggacacggctgtgtattactgtaccagagataactatggccgcaactatgattatggcatggattattggggccagggcaccctggtgaccgtgagcagcggtggtggcggttccggcggtggcggttccggtggtggcggttccgaaattgtgttgacacagtctccagccaccctgtctttgtctccaggggaaagagccaccctcacctgcagcgccaccagcagtgttagctacatccactggtatcaacagaggcctggccagagccccaggctcctcatctatagcacctccaacctggccagcggcatcccagccaggttcagtggcagtgggtctgggagcgactacactctcaccatcagcagcctagagcctgaagattttgcagtttattactgtcagcagcgtagcagctcccctttcacctttggcagcggcaccaaagtggaaattaaaaagcacctgtgtccttctccactgttccccggccctagcaagcctttctgggtcctggtggtggtgggcggagtgctggcctgctacagcctgctggtgaccgtggcctttatcatcttctgggtgaggagtaagaggagcaggctcctgcacagtgactacatgaacatgactcctagaagacctgggcctaccagaaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgc

SEQ ID NO: 95 - DNA, 1391 bp, synthetic construct
  source 1..1391: mol_type = other DNA
atggccttaccagtgaccgccttgctcctgccgctggccttgctgctccacgccgccaggccgcaggtgcagctggttgaatctggcggcggacttgtgaagcctggcggatctctgagactgagctgtgccgccagcggcttcacctttagcacatacgccatgagctggatcagacaggcccctggcaaaggcctggaatgggtggcgtctattggcagagccggcagcacctactacagcgactctgtgaagggcagattcaccatcagccgggacaacgccaagaacagcctgtacctgcagatgaactccctgagagccgaggacaccgccgtgtactattgtgccagaggacccatctacaacgactacgacgagttcgcctattggggccagggcacactggtcacagtttctagcggcggtggcggaagcggaggcggtggctccggtggcggaggcagcgaaattgtgctgacacagagccccgccacactgtcactttctccaggcgaaagagccacactgagctgcaagagcagccagagcgtgctgtactccagcaaccagaagaactacctggcctggtatcagcagaagcccggccaagctcctcggctgctgatctattgggccagcacaagagagagcggcatccctgccagattttctggcagcggctctggcaccgatttcaccctgaccataagcagcctggaacctgaggacttcgccgtgtattactgccaccagtacctgagcagcctgacctttggcggaggcaccaaggtggaaatcaagcggaagcacctgtgtccttctccactgttccccggccctagcaagcctttctgggtcctggtggtggtgggcggagtgctggcctgctacagcctgctggtgaccgtggcctttatcatcttctgggtgaggagtaagaggagcaggctcctgcacagtgactacatgaacatgactcctagaagacctgggcctaccagaaagcattaccagccctatgccccaccacgcgacttcgcagcctatcgctccagagtgaagttcagcaggagcgcagacgccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgc

SEQ ID NO: 96 - DNA, 3469 bp, synthetic construct
  source 1..3469: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtggctatgccatgagctgggtccgccaggctccagggaaggggctggagtgggtctcaaccattagtagtggcggaacctacatatactaccccgactcagtgaagggccgattcaccatctccagagacaacgccaagaactcactgtatctgcaaatgaacagcctgagagccgaggacacggccgtgtattactgtgcgagacttgggggggataattactacgaatacttcgatgtctggggcaaagggaccacggtcaccgtctcctccggcggtggcggatccggcggtggcggatccggcggtggcggatccgacattgtgctgacccagtctccagcctccttggccgtgtctccaggacagagggccaccatcacctgcagagccagtaagagtgtcagtaccagcggatactcctacatgcactggtatcagcagaaaccaggacaacctcctaaactcctgatttacctggcatccaatctggagagcggggtcccagccaggttcagcggcagtgggtctgggaccgatttcaccctcacaattaatcctgtggaagctaatgatactgcaaattattactgtcagcacagtagggagctgcctttcacattcggcggagggaccaaggtggagatcaaacgaactacaacaacccctgcccccagacctcctaccccagcccctacaattgccagccagcctctgagcctgaggcccgaggcttgtagacctgctgctggcggagccgtgcacaccagaggactggatttcgcctgcgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataagtttaaactgccagaacatttctctggcctaactggccggtaccggcttcattttttccatttactgcagaggcttcattttttccatttactgcagaggcttcattttttccatttactgcagaggcttcattttttccatttactgcagaggcttcattttttccatttactgcagaggcttcattttttccatttactgcagaactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttgataa

SEQ ID NO: 97 - DNA, 3388 bp, synthetic construct
  source 1..3388: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtggctatgccatgagctgggtccgccaggctccagggaaggggctggagtgggtctcaaccattagtagtggcggaacctacatatactaccccgactcagtgaagggccgattcaccatctccagagacaacgccaagaactcactgtatctgcaaatgaacagcctgagagccgaggacacggccgtgtattactgtgcgagacttgggggggataattactacgaatacttcgatgtctggggcaaagggaccacggtcaccgtctcctccggcggtggcggatccggcggtggcggatccggcggtggcggatccgacattgtgctgacccagtctccagcctccttggccgtgtctccaggacagagggccaccatcacctgcagagccagtaagagtgtcagtaccagcggatactcctacatgcactggtatcagcagaaaccaggacaacctcctaaactcctgatttacctggcatccaatctggagagcggggtcccagccaggttcagcggcagtgggtctgggaccgatttcaccctcacaattaatcctgtggaagctaatgatactgcaaattattactgtcagcacagtagggagctgcctttcacattcggcggagggaccaaggtggagatcaaacgaactacaacaacccctgcccccagacctcctaccccagcccctacaattgccagccagcctctgagcctgaggcccgaggcttgtagacctgctgctggcggagccgtgcacaccagaggactggatttcgcctgcgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataagtttaaactgccagaacatttctctggcctaactggccggtaccggcttcattttttccatttactgcagaggcttcattttttccatttactgcagaggcttcattttttccatttactgcagaactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttgataa

SEQ ID NO: 98 - DNA, 3487 bp, synthetic construct
  source 1..3487: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtggctatgccatgagctgggtccgccaggctccagggaaggggctggagtgggtctcaaccattagtagtggcggaacctacatatactaccccgactcagtgaagggccgattcaccatctccagagacaacgccaagaactcactgtatctgcaaatgaacagcctgagagccgaggacacggccgtgtattactgtgcgagacttgggggggataattactacgaatacttcgatgtctggggcaaagggaccacggtcaccgtctcctccggcggtggcggatccggcggtggcggatccggcggtggcggatccgacattgtgctgacccagtctccagcctccttggccgtgtctccaggacagagggccaccatcacctgcagagccagtaagagtgtcagtaccagcggatactcctacatgcactggtatcagcagaaaccaggacaacctcctaaactcctgatttacctggcatccaatctggagagcggggtcccagccaggttcagcggcagtgggtctgggaccgatttcaccctcacaattaatcctgtggaagctaatgatactgcaaattattactgtcagcacagtagggagctgcctttcacattcggcggagggaccaaggtggagatcaaacgaactacaacaacccctgcccccagacctcctaccccagcccctacaattgccagccagcctctgagcctgaggcccgaggcttgtagacctgctgctggcggagccgtgcacaccagaggactggatttcgcctgcgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataagtttaaactgccagaacatttctctggcctaactggccggtaccggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttgataa

SEQ ID NO: 99 - DNA, 3397 bp, synthetic construct
  source 1..3397: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtggctatgccatgagctgggtccgccaggctccagggaaggggctggagtgggtctcaaccattagtagtggcggaacctacatatactaccccgactcagtgaagggccgattcaccatctccagagacaacgccaagaactcactgtatctgcaaatgaacagcctgagagccgaggacacggccgtgtattactgtgcgagacttgggggggataattactacgaatacttcgatgtctggggcaaagggaccacggtcaccgtctcctccggcggtggcggatccggcggtggcggatccggcggtggcggatccgacattgtgctgacccagtctccagcctccttggccgtgtctccaggacagagggccaccatcacctgcagagccagtaagagtgtcagtaccagcggatactcctacatgcactggtatcagcagaaaccaggacaacctcctaaactcctgatttacctggcatccaatctggagagcggggtcccagccaggttcagcggcagtgggtctgggaccgatttcaccctcacaattaatcctgtggaagctaatgatactgcaaattattactgtcagcacagtagggagctgcctttcacattcggcggagggaccaaggtggagatcaaacgaactacaacaacccctgcccccagacctcctaccccagcccctacaattgccagccagcctctgagcctgaggcccgaggcttgtagacctgctgctggcggagccgtgcacaccagaggactggatttcgcctgcgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataagtttaaactgccagaacatttctctggcctaactggccggtaccggaggaaaaactgtttcatacagaaggcgtggaggaaaaactgtttcatacagaaggcgtggaggaaaaactgtttcatacagaaggcgtactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttgataa

SEQ ID NO: 100 - DNA, 3385 bp, synthetic construct
  source 1..3385: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtggctatgccatgagctgggtccgccaggctccagggaaggggctggagtgggtctcaaccattagtagtggcggaacctacatatactaccccgactcagtgaagggccgattcaccatctccagagacaacgccaagaactcactgtatctgcaaatgaacagcctgagagccgaggacacggccgtgtattactgtgcgagacttgggggggataattactacgaatacttcgatgtctggggcaaagggaccacggtcaccgtctcctccggcggtggcggatccggcggtggcggatccggcggtggcggatccgacattgtgctgacccagtctccagcctccttggccgtgtctccaggacagagggccaccatcacctgcagagccagtaagagtgtcagtaccagcggatactcctacatgcactggtatcagcagaaaccaggacaacctcctaaactcctgatttacctggcatccaatctggagagcggggtcccagccaggttcagcggcagtgggtctgggaccgatttcaccctcacaattaatcctgtggaagctaatgatactgcaaattattactgtcagcacagtagggagctgcctttcacattcggcggagggaccaaggtggagatcaaacgaactaagcacctgtgtccttctccactgttccccggccctagcaagcctttctgggtcctggtggtggtgggcggagtgctggcctgctacagcctgctggtgaccgtggcctttatcatcttctgggtgaggagtaagaggagcaggctcctgcacagtgactacatgaacatgactcctagaagacctgggcctaccagaaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataagtttaaactgccagaacatttctctggcctaactggccggtaccggcttcattttttccatttactgcagaggcttcattttttccatttactgcagaggcttcattttttccatttactgcagaggcttcattttttccatttactgcagaggcttcattttttccatttactgcagaggcttcattttttccatttactgcagaactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttgataa

SEQ ID NO: 101 - DNA, 3304 bp, synthetic construct
  source 1..3304: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtggctatgccatgagctgggtccgccaggctccagggaaggggctggagtgggtctcaaccattagtagtggcggaacctacatatactaccccgactcagtgaagggccgattcaccatctccagagacaacgccaagaactcactgtatctgcaaatgaacagcctgagagccgaggacacggccgtgtattactgtgcgagacttgggggggataattactacgaatacttcgatgtctggggcaaagggaccacggtcaccgtctcctccggcggtggcggatccggcggtggcggatccggcggtggcggatccgacattgtgctgacccagtctccagcctccttggccgtgtctccaggacagagggccaccatcacctgcagagccagtaagagtgtcagtaccagcggatactcctacatgcactggtatcagcagaaaccaggacaacctcctaaactcctgatttacctggcatccaatctggagagcggggtcccagccaggttcagcggcagtgggtctgggaccgatttcaccctcacaattaatcctgtggaagctaatgatactgcaaattattactgtcagcacagtagggagctgcctttcacattcggcggagggaccaaggtggagatcaaacgaactaagcacctgtgtccttctccactgttccccggccctagcaagcctttctgggtcctggtggtggtgggcggagtgctggcctgctacagcctgctggtgaccgtggcctttatcatcttctgggtgaggagtaagaggagcaggctcctgcacagtgactacatgaacatgactcctagaagacctgggcctaccagaaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataagtttaaactgccagaacatttctctggcctaactggccggtaccggcttcattttttccatttactgcagaggcttcattttttccatttactgcagaggcttcattttttccatttactgcagaactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttgataa

SEQ ID NO: 102 - DNA, 3403 bp, synthetic construct
  source 1..3403: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtggctatgccatgagctgggtccgccaggctccagggaaggggctggagtgggtctcaaccattagtagtggcggaacctacatatactaccccgactcagtgaagggccgattcaccatctccagagacaacgccaagaactcactgtatctgcaaatgaacagcctgagagccgaggacacggccgtgtattactgtgcgagacttgggggggataattactacgaatacttcgatgtctggggcaaagggaccacggtcaccgtctcctccggcggtggcggatccggcggtggcggatccggcggtggcggatccgacattgtgctgacccagtctccagcctccttggccgtgtctccaggacagagggccaccatcacctgcagagccagtaagagtgtcagtaccagcggatactcctacatgcactggtatcagcagaaaccaggacaacctcctaaactcctgatttacctggcatccaatctggagagcggggtcccagccaggttcagcggcagtgggtctgggaccgatttcaccctcacaattaatcctgtggaagctaatgatactgcaaattattactgtcagcacagtagggagctgcctttcacattcggcggagggaccaaggtggagatcaaacgaactaagcacctgtgtccttctccactgttccccggccctagcaagcctttctgggtcctggtggtggtgggcggagtgctggcctgctacagcctgctggtgaccgtggcctttatcatcttctgggtgaggagtaagaggagcaggctcctgcacagtgactacatgaacatgactcctagaagacctgggcctaccagaaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataagtttaaactgccagaacatttctctggcctaactggccggtaccggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttgataa

SEQ ID NO: 103 - DNA, 3313 bp, synthetic construct
  source 1..3313: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtggctatgccatgagctgggtccgccaggctccagggaaggggctggagtgggtctcaaccattagtagtggcggaacctacatatactaccccgactcagtgaagggccgattcaccatctccagagacaacgccaagaactcactgtatctgcaaatgaacagcctgagagccgaggacacggccgtgtattactgtgcgagacttgggggggataattactacgaatacttcgatgtctggggcaaagggaccacggtcaccgtctcctccggcggtggcggatccggcggtggcggatccggcggtggcggatccgacattgtgctgacccagtctccagcctccttggccgtgtctccaggacagagggccaccatcacctgcagagccagtaagagtgtcagtaccagcggatactcctacatgcactggtatcagcagaaaccaggacaacctcctaaactcctgatttacctggcatccaatctggagagcggggtcccagccaggttcagcggcagtgggtctgggaccgatttcaccctcacaattaatcctgtggaagctaatgatactgcaaattattactgtcagcacagtagggagctgcctttcacattcggcggagggaccaaggtggagatcaaacgaactaagcacctgtgtccttctccactgttccccggccctagcaagcctttctgggtcctggtggtggtgggcggagtgctggcctgctacagcctgctggtgaccgtggcctttatcatcttctgggtgaggagtaagaggagcaggctcctgcacagtgactacatgaacatgactcctagaagacctgggcctaccagaaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataagtttaaactgccagaacatttctctggcctaactggccggtaccggaggaaaaactgtttcatacagaaggcgtggaggaaaaactgtttcatacagaaggcgtggaggaaaaactgtttcatacagaaggcgtactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttgataa

SEQ ID NO: 104 - DNA, 3385 bp, synthetic construct
  source 1..3385: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtggctatgccatgagctgggtccgccaggctccagggaaggggctggagtgggtctcaaccattagtagtggcggaacctacatatactaccccgactcagtgaagggccgattcaccatctccagagacaacgccaagaactcactgtatctgcaaatgaacagcctgagagccgaggacacggccgtgtattactgtgcgagacttgggggggataattactacgaatacttcgatgtctggggcaaagggaccacggtcaccgtctcctccggcggtggcggatccggcggtggcggatccggcggtggcggatccgacattgtgctgacccagtctccagcctccttggccgtgtctccaggacagagggccaccatcacctgcagagccagtaagagtgtcagtaccagcggatactcctacatgcactggtatcagcagaaaccaggacaacctcctaaactcctgatttacctggcatccaatctggagagcggggtcccagccaggttcagcggcagtgggtctgggaccgatttcaccctcacaattaatcctgtggaagctaatgatactgcaaattattactgtcagcacagtagggagctgcctttcacattcggcggagggaccaaggtggagatcaaacgaactaagcacctgtgtccttctccactgttccccggccctagcaagcctttctgggtcctggtggtggtgggcggagtgctggcctgctacagcctgctggtgaccgtggcctttatcatcttctgggtgaggagtaagaggagcaggctcctgcacagtgactacatgaacatgactcctagaagacctgggcctaccagaaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgctgataagtttaaactgccagaacattactctggcctaactggccggtaccggcttcattttttccatttactgcagaggcttcattttttccatttactgcagaggcttcattttttccatttactgcagaggcttcattttttccatttactgcagaggcttcattttttccatttactgcagaggcttcattttttccatttactgcagaactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttgataa

SEQ ID NO: 105 - DNA, 3304 bp, synthetic construct
  source 1..3304: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtggctatgccatgagctgggtccgccaggctccagggaaggggctggagtgggtctcaaccattagtagtggcggaacctacatatactaccccgactcagtgaagggccgattcaccatctccagagacaacgccaagaactcactgtatctgcaaatgaacagcctgagagccgaggacacggccgtgtattactgtgcgagacttgggggggataattactacgaatacttcgatgtctggggcaaagggaccacggtcaccgtctcctccggcggtggcggatccggcggtggcggatccggcggtggcggatccgacattgtgctgacccagtctccagcctccttggccgtgtctccaggacagagggccaccatcacctgcagagccagtaagagtgtcagtaccagcggatactcctacatgcactggtatcagcagaaaccaggacaacctcctaaactcctgatttacctggcatccaatctggagagcggggtcccagccaggttcagcggcagtgggtctgggaccgatttcaccctcacaattaatcctgtggaagctaatgatactgcaaattattactgtcagcacagtagggagctgcctttcacattcggcggagggaccaaggtggagatcaaacgaactaagcacctgtgtccttctccactgttccccggccctagcaagcctttctgggtcctggtggtggtgggcggagtgctggcctgctacagcctgctggtgaccgtggcctttatcatcttctgggtgaggagtaagaggagcaggctcctgcacagtgactacatgaacatgactcctagaagacctgggcctaccagaaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgctgataagtttaaactgccagaacattactctggcctaactggccggtaccggcttcattttttccatttactgcagaggcttcattttttccatttactgcagaggcttcattttttccatttactgcagaactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttgataa

SEQ ID NO: 106 - DNA, 3403 bp, synthetic construct
  source 1..3403: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtggctatgccatgagctgggtccgccaggctccagggaaggggctggagtgggtctcaaccattagtagtggcggaacctacatatactaccccgactcagtgaagggccgattcaccatctccagagacaacgccaagaactcactgtatctgcaaatgaacagcctgagagccgaggacacggccgtgtattactgtgcgagacttgggggggataattactacgaatacttcgatgtctggggcaaagggaccacggtcaccgtctcctccggcggtggcggatccggcggtggcggatccggcggtggcggatccgacattgtgctgacccagtctccagcctccttggccgtgtctccaggacagagggccaccatcacctgcagagccagtaagagtgtcagtaccagcggatactcctacatgcactggtatcagcagaaaccaggacaacctcctaaactcctgatttacctggcatccaatctggagagcggggtcccagccaggttcagcggcagtgggtctgggaccgatttcaccctcacaattaatcctgtggaagctaatgatactgcaaattattactgtcagcacagtagggagctgcctttcacattcggcggagggaccaaggtggagatcaaacgaactaagcacctgtgtccttctccactgttccccggccctagcaagcctttctgggtcctggtggtggtgggcggagtgctggcctgctacagcctgctggtgaccgtggcctttatcatcttctgggtgaggagtaagaggagcaggctcctgcacagtgactacatgaacatgactcctagaagacctgggcctaccagaaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgctgataagtttaaactgccagaacattactctggcctaactggccggtaccggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttgataa

SEQ ID NO: 107 - DNA, 3313 bp, synthetic construct
  source 1..3313: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtggctatgccatgagctgggtccgccaggctccagggaaggggctggagtgggtctcaaccattagtagtggcggaacctacatatactaccccgactcagtgaagggccgattcaccatctccagagacaacgccaagaactcactgtatctgcaaatgaacagcctgagagccgaggacacggccgtgtattactgtgcgagacttgggggggataattactacgaatacttcgatgtctggggcaaagggaccacggtcaccgtctcctccggcggtggcggatccggcggtggcggatccggcggtggcggatccgacattgtgctgacccagtctccagcctccttggccgtgtctccaggacagagggccaccatcacctgcagagccagtaagagtgtcagtaccagcggatactcctacatgcactggtatcagcagaaaccaggacaacctcctaaactcctgatttacctggcatccaatctggagagcggggtcccagccaggttcagcggcagtgggtctgggaccgatttcaccctcacaattaatcctgtggaagctaatgatactgcaaattattactgtcagcacagtagggagctgcctttcacattcggcggagggaccaaggtggagatcaaacgaactaagcacctgtgtccttctccactgttccccggccctagcaagcctttctgggtcctggtggtggtgggcggagtgctggcctgctacagcctgctggtgaccgtggcctttatcatcttctgggtgaggagtaagaggagcaggctcctgcacagtgactacatgaacatgactcctagaagacctgggcctaccagaaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgctgataagtttaaactgccagaacattactctggcctaactggccggtaccggaggaaaaactgtttcatacagaaggcgtggaggaaaaactgtttcatacagaaggcgtggaggaaaaactgtttcatacagaaggcgtactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttgataa

SEQ ID NO: 108 - DNA, 3947 bp, synthetic construct
  source 1..3947: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtaggtatggcatgagctgggtccgccaggctccagggaagaggctggagtgggtctcaaccattagtggcggaggcacctacatatactacccagactcagtgaagggccgattcaccatctccagagacaacgccaagaacaccctgtatctgcaaatgaacagcctgagagccgaggacacggctgtgtattactgtaccagagataactatggccgcaactatgattatggcatggattattggggccagggcaccctggtgaccgtgagcagcggtggtggcggttccggcggtggcggttccggtggtggcggttccgaaattgtgttgacacagtctccagccaccctgtctttgtctccaggggaaagagccaccctcacctgcagcgccaccagcagtgttagctacatccactggtatcaacagaggcctggccagagccccaggctcctcatctatagcacctccaacctggccagcggcatcccagccaggttcagtggcagtgggtctgggagcgactacactctcaccatcagcagcctagagcctgaagattttgcagtttattactgtcagcagcgtagcagctcccctttcacctttggcagcggcaccaaagtggaaattaaaacaacaacccctgcccccagacctcctaccccagcccctacaattgccagccagcctctgagcctgaggcccgaggcttgtagacctgctgctggcggagccgtgcacaccagaggactggatttcgcctgcgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataagtttaaactgccagaacatttctctggcctaactggccggtaccggcttcattttttccatttactgcagaggcttcattttttccatttactgcagaggcttcattttttccatttactgcagaggcttcattttttccatttactgcagaggcttcattttttccatttactgcagaggcttcattttttccatttactgcagaactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtctggaagcggtgagggaagaggtagcctgctcacgtgcggtgatgtggaggagaaccctgggcctatgctctgtttgggctggatatttctttggcttgttgcaggagagcgaattaaaggatttaatatttcaggttgttccacaaaaaaactcctttggacatattctacaaggagtgaagaggaatttgtcttattttgtgatttaccagagccacagaaatcacatttctgccacagaaatcgactctcaccaaaacaagtccctgagcacctgcccttcatgggtagtaacgacctatctgatgtccaatggtaccaacaaccttcgaatggagatccattagaggacattaggaaaagctatcctcacatcattcaggacaaatgtacccttcactttttgaccccaggggtgaataattctgggtcatatatttgtagacccaagatgattaagagcccctatgatgtagcctgttgtgtcaagatgattttagaag

SEQ ID NO: 109 - DNA, 3370 bp, synthetic construct
  source 1..3370: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtaggtatggcatgagctgggtccgccaggctccagggaagaggctggagtgggtctcaaccattagtggcggaggcacctacatatactacccagactcagtgaagggccgattcaccatctccagagacaacgccaagaacaccctgtatctgcaaatgaacagcctgagagccgaggacacggctgtgtattactgtaccagagataactatggccgcaactatgattatggcatggattattggggccagggcaccctggtgaccgtgagcagcggtggtggcggttccggcggtggcggttccggtggtggcggttccgaaattgtgttgacacagtctccagccaccctgtctttgtctccaggggaaagagccaccctcacctgcagcgccaccagcagtgttagctacatccactggtatcaacagaggcctggccagagccccaggctcctcatctatagcacctccaacctggccagcggcatcccagccaggttcagtggcagtgggtctgggagcgactacactctcaccatcagcagcctagagcctgaagattttgcagtttattactgtcagcagcgtagcagctcccctttcacctttggcagcggcaccaaagtggaaattaaaacaacaacccctgcccccagacctcctaccccagcccctacaattgccagccagcctctgagcctgaggcccgaggcttgtagacctgctgctggcggagccgtgcacaccagaggactggatttcgcctgcgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataagtttaaactgccagaacatttctctggcctaactggccggtaccggcttcattttttccatttactgcagaggcttcattttttccatttactgcagaggcttcattttttccatttactgcagaactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttgataa

SEQ ID NO: 110 - DNA, 3469 bp, synthetic construct
  source 1..3469: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtaggtatggcatgagctgggtccgccaggctccagggaagaggctggagtgggtctcaaccattagtggcggaggcacctacatatactacccagactcagtgaagggccgattcaccatctccagagacaacgccaagaacaccctgtatctgcaaatgaacagcctgagagccgaggacacggctgtgtattactgtaccagagataactatggccgcaactatgattatggcatggattattggggccagggcaccctggtgaccgtgagcagcggtggtggcggttccggcggtggcggttccggtggtggcggttccgaaattgtgttgacacagtctccagccaccctgtctttgtctccaggggaaagagccaccctcacctgcagcgccaccagcagtgttagctacatccactggtatcaacagaggcctggccagagccccaggctcctcatctatagcacctccaacctggccagcggcatcccagccaggttcagtggcagtgggtctgggagcgactacactctcaccatcagcagcctagagcctgaagattttgcagtttattactgtcagcagcgtagcagctcccctttcacctttggcagcggcaccaaagtggaaattaaaacaacaacccctgcccccagacctcctaccccagcccctacaattgccagccagcctctgagcctgaggcccgaggcttgtagacctgctgctggcggagccgtgcacaccagaggactggatttcgcctgcgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataagtttaaactgccagaacatttctctggcctaactggccggtaccggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttgataa

SEQ ID NO: 111 - DNA, 3379 bp, synthetic construct
  source 1..3379: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtaggtatggcatgagctgggtccgccaggctccagggaagaggctggagtgggtctcaaccattagtggcggaggcacctacatatactacccagactcagtgaagggccgattcaccatctccagagacaacgccaagaacaccctgtatctgcaaatgaacagcctgagagccgaggacacggctgtgtattactgtaccagagataactatggccgcaactatgattatggcatggattattggggccagggcaccctggtgaccgtgagcagcggtggtggcggttccggcggtggcggttccggtggtggcggttccgaaattgtgttgacacagtctccagccaccctgtctttgtctccaggggaaagagccaccctcacctgcagcgccaccagcagtgttagctacatccactggtatcaacagaggcctggccagagccccaggctcctcatctatagcacctccaacctggccagcggcatcccagccaggttcagtggcagtgggtctgggagcgactacactctcaccatcagcagcctagagcctgaagattttgcagtttattactgtcagcagcgtagcagctcccctttcacctttggcagcggcaccaaagtggaaattaaaacaacaacccctgcccccagacctcctaccccagcccctacaattgccagccagcctctgagcctgaggcccgaggcttgtagacctgctgctggcggagccgtgcacaccagaggactggatttcgcctgcgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataagtttaaactgccagaacatttctctggcctaactggccggtaccggaggaaaaactgtttcatacagaaggcgtggaggaaaaactgtttcatacagaaggcgtggaggaaaaactgtttcatacagaaggcgtactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttgataa

SEQ ID NO: 112 - DNA, 3367 bp, synthetic construct
  source 1..3367: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtaggtatggcatgagctgggtccgccaggctccagggaagaggctggagtgggtctcaaccattagtggcggaggcacctacatatactacccagactcagtgaagggccgattcaccatctccagagacaacgccaagaacaccctgtatctgcaaatgaacagcctgagagccgaggacacggctgtgtattactgtaccagagataactatggccgcaactatgattatggcatggattattggggccagggcaccctggtgaccgtgagcagcggtggtggcggttccggcggtggcggttccggtggtggcggttccgaaattgtgttgacacagtctccagccaccctgtctttgtctccaggggaaagagccaccctcacctgcagcgccaccagcagtgttagctacatccactggtatcaacagaggcctggccagagccccaggctcctcatctatagcacctccaacctggccagcggcatcccagccaggttcagtggcagtgggtctgggagcgactacactctcaccatcagcagcctagagcctgaagattttgcagtttattactgtcagcagcgtagcagctcccctttcacctttggcagcggcaccaaagtggaaattaaaaagcacctgtgtccttctccactgttccccggccctagcaagcctttctgggtcctggtggtggtgggcggagtgctggcctgctacagcctgctggtgaccgtggcctttatcatcttctgggtgaggagtaagaggagcaggctcctgcacagtgactacatgaacatgactcctagaagacctgggcctaccagaaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataagtttaaactgccagaacatttctctggcctaactggccggtaccggcttcattttttccatttactgcagaggcttcattttttccatttactgcagaggcttcattttttccatttactgcagaggcttcattttttccatttactgcagaggcttcattttttccatttactgcagaggcttcattttttccatttactgcagaactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttgataa

SEQ ID NO: 113 - DNA, 3286 bp, synthetic construct
  source 1..3286: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtaggtatggcatgagctgggtccgccaggctccagggaagaggctggagtgggtctcaaccattagtggcggaggcacctacatatactacccagactcagtgaagggccgattcaccatctccagagacaacgccaagaacaccctgtatctgcaaatgaacagcctgagagccgaggacacggctgtgtattactgtaccagagataactatggccgcaactatgattatggcatggattattggggccagggcaccctggtgaccgtgagcagcggtggtggcggttccggcggtggcggttccggtggtggcggttccgaaattgtgttgacacagtctccagccaccctgtctttgtctccaggggaaagagccaccctcacctgcagcgccaccagcagtgttagctacatccactggtatcaacagaggcctggccagagccccaggctcctcatctatagcacctccaacctggccagcggcatcccagccaggttcagtggcagtgggtctgggagcgactacactctcaccatcagcagcctagagcctgaagattttgcagtttattactgtcagcagcgtagcagctcccctttcacctttggcagcggcaccaaagtggaaattaaaaagcacctgtgtccttctccactgttccccggccctagcaagcctttctgggtcctggtggtggtgggcggagtgctggcctgctacagcctgctggtgaccgtggcctttatcatcttctgggtgaggagtaagaggagcaggctcctgcacagtgactacatgaacatgactcctagaagacctgggcctaccagaaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataagtttaaactgccagaacatttctctggcctaactggccggtaccggcttcattttttccatttactgcagaggcttcattttttccatttactgcagaggcttcattttttccatttactgcagaactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttgataa

SEQ ID NO: 114 - DNA, 3385 bp, synthetic construct
  source 1..3385: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtaggtatggcatgagctgggtccgccaggctccagggaagaggctggagtgggtctcaaccattagtggcggaggcacctacatatactacccagactcagtgaagggccgattcaccatctccagagacaacgccaagaacaccctgtatctgcaaatgaacagcctgagagccgaggacacggctgtgtattactgtaccagagataactatggccgcaactatgattatggcatggattattggggccagggcaccctggtgaccgtgagcagcggtggtggcggttccggcggtggcggttccggtggtggcggttccgaaattgtgttgacacagtctccagccaccctgtctttgtctccaggggaaagagccaccctcacctgcagcgccaccagcagtgttagctacatccactggtatcaacagaggcctggccagagccccaggctcctcatctatagcacctccaacctggccagcggcatcccagccaggttcagtggcagtgggtctgggagcgactacactctcaccatcagcagcctagagcctgaagattttgcagtttattactgtcagcagcgtagcagctcccctttcacctttggcagcggcaccaaagtggaaattaaaaagcacctgtgtccttctccactgttccccggccctagcaagcctttctgggtcctggtggtggtgggcggagtgctggcctgctacagcctgctggtgaccgtggcctttatcatcttctgggtgaggagtaagaggagcaggctcctgcacagtgactacatgaacatgactcctagaagacctgggcctaccagaaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataagtttaaactgccagaacatttctctggcctaactggccggtaccggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttgataa

SEQ ID NO: 115 - DNA, 3295 bp, synthetic construct
  source 1..3295: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtaggtatggcatgagctgggtccgccaggctccagggaagaggctggagtgggtctcaaccattagtggcggaggcacctacatatactacccagactcagtgaagggccgattcaccatctccagagacaacgccaagaacaccctgtatctgcaaatgaacagcctgagagccgaggacacggctgtgtattactgtaccagagataactatggccgcaactatgattatggcatggattattggggccagggcaccctggtgaccgtgagcagcggtggtggcggttccggcggtggcggttccggtggtggcggttccgaaattgtgttgacacagtctccagccaccctgtctttgtctccaggggaaagagccaccctcacctgcagcgccaccagcagtgttagctacatccactggtatcaacagaggcctggccagagccccaggctcctcatctatagcacctccaacctggccagcggcatcccagccaggttcagtggcagtgggtctgggagcgactacactctcaccatcagcagcctagagcctgaagattttgcagtttattactgtcagcagcgtagcagctcccctttcacctttggcagcggcaccaaagtggaaattaaaaagcacctgtgtccttctccactgttccccggccctagcaagcctttctgggtcctggtggtggtgggcggagtgctggcctgctacagcctgctggtgaccgtggcctttatcatcttctgggtgaggagtaagaggagcaggctcctgcacagtgactacatgaacatgactcctagaagacctgggcctaccagaaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataagtttaaactgccagaacatttctctggcctaactggccggtaccggaggaaaaactgtttcatacagaaggcgtggaggaaaaactgtttcatacagaaggcgtggaggaaaaactgtttcatacagaaggcgtactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttgataa

SEQ ID NO: 116 - DNA, 3367 bp, synthetic construct
  source 1..3367: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtaggtatggcatgagctgggtccgccaggctccagggaagaggctggagtgggtctcaaccattagtggcggaggcacctacatatactacccagactcagtgaagggccgattcaccatctccagagacaacgccaagaacaccctgtatctgcaaatgaacagcctgagagccgaggacacggctgtgtattactgtaccagagataactatggccgcaactatgattatggcatggattattggggccagggcaccctggtgaccgtgagcagcggtggtggcggttccggcggtggcggttccggtggtggcggttccgaaattgtgttgacacagtctccagccaccctgtctttgtctccaggggaaagagccaccctcacctgcagcgccaccagcagtgttagctacatccactggtatcaacagaggcctggccagagccccaggctcctcatctatagcacctccaacctggccagcggcatcccagccaggttcagtggcagtgggtctgggagcgactacactctcaccatcagcagcctagagcctgaagattttgcagtttattactgtcagcagcgtagcagctcccctttcacctttggcagcggcaccaaagtggaaattaaaaagcacctgtgtccttctccactgttccccggccctagcaagcctttctgggtcctggtggtggtgggcggagtgctggcctgctacagcctgctggtgaccgtggcctttatcatcttctgggtgaggagtaagaggagcaggctcctgcacagtgactacatgaacatgactcctagaagacctgggcctaccagaaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgctgataagtttaaactgccagaacattactctggcctaactggccggtaccggcttcattttttccatttactgcagaggcttcattttttccatttactgcagaggcttcattttttccatttactgcagaggcttcattttttccatttactgcagaggcttcattttttccatttactgcagaggcttcattttttccatttactgcagaactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttgataa

SEQ ID NO: 117 - DNA, 3286 bp, synthetic construct
  source 1..3286: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtaggtatggcatgagctgggtccgccaggctccagggaagaggctggagtgggtctcaaccattagtggcggaggcacctacatatactacccagactcagtgaagggccgattcaccatctccagagacaacgccaagaacaccctgtatctgcaaatgaacagcctgagagccgaggacacggctgtgtattactgtaccagagataactatggccgcaactatgattatggcatggattattggggccagggcaccctggtgaccgtgagcagcggtggtggcggttccggcggtggcggttccggtggtggcggttccgaaattgtgttgacacagtctccagccaccctgtctttgtctccaggggaaagagccaccctcacctgcagcgccaccagcagtgttagctacatccactggtatcaacagaggcctggccagagccccaggctcctcatctatagcacctccaacctggccagcggcatcccagccaggttcagtggcagtgggtctgggagcgactacactctcaccatcagcagcctagagcctgaagattttgcagtttattactgtcagcagcgtagcagctcccctttcacctttggcagcggcaccaaagtggaaattaaaaagcacctgtgtccttctccactgttccccggccctagcaagcctttctgggtcctggtggtggtgggcggagtgctggcctgctacagcctgctggtgaccgtggcctttatcatcttctgggtgaggagtaagaggagcaggctcctgcacagtgactacatgaacatgactcctagaagacctgggcctaccagaaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgctgataagtttaaactgccagaacattactctggcctaactggccggtaccggcttcattttttccatttactgcagaggcttcattttttccatttactgcagaggcttcattttttccatttactgcagaactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttgataa

SEQ ID NO: 118 - DNA, 3385 bp, synthetic construct
  source 1..3385: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtaggtatggcatgagctgggtccgccaggctccagggaagaggctggagtgggtctcaaccattagtggcggaggcacctacatatactacccagactcagtgaagggccgattcaccatctccagagacaacgccaagaacaccctgtatctgcaaatgaacagcctgagagccgaggacacggctgtgtattactgtaccagagataactatggccgcaactatgattatggcatggattattggggccagggcaccctggtgaccgtgagcagcggtggtggcggttccggcggtggcggttccggtggtggcggttccgaaattgtgttgacacagtctccagccaccctgtctttgtctccaggggaaagagccaccctcacctgcagcgccaccagcagtgttagctacatccactggtatcaacagaggcctggccagagccccaggctcctcatctatagcacctccaacctggccagcggcatcccagccaggttcagtggcagtgggtctgggagcgactacactctcaccatcagcagcctagagcctgaagattttgcagtttattactgtcagcagcgtagcagctcccctttcacctttggcagcggcaccaaagtggaaattaaaaagcacctgtgtccttctccactgttccccggccctagcaagcctttctgggtcctggtggtggtgggcggagtgctggcctgctacagcctgctggtgaccgtggcctttatcatcttctgggtgaggagtaagaggagcaggctcctgcacagtgactacatgaacatgactcctagaagacctgggcctaccagaaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgctgataagtttaaactgccagaacattactctggcctaactggccggtaccggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttgataa

SEQ ID NO: 119 - DNA, 3295 bp, synthetic construct
  source 1..3295: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtaggtatggcatgagctgggtccgccaggctccagggaagaggctggagtgggtctcaaccattagtggcggaggcacctacatatactacccagactcagtgaagggccgattcaccatctccagagacaacgccaagaacaccctgtatctgcaaatgaacagcctgagagccgaggacacggctgtgtattactgtaccagagataactatggccgcaactatgattatggcatggattattggggccagggcaccctggtgaccgtgagcagcggtggtggcggttccggcggtggcggttccggtggtggcggttccgaaattgtgttgacacagtctccagccaccctgtctttgtctccaggggaaagagccaccctcacctgcagcgccaccagcagtgttagctacatccactggtatcaacagaggcctggccagagccccaggctcctcatctatagcacctccaacctggccagcggcatcccagccaggttcagtggcagtgggtctgggagcgactacactctcaccatcagcagcctagagcctgaagattttgcagtttattactgtcagcagcgtagcagctcccctttcacctttggcagcggcaccaaagtggaaattaaaaagcacctgtgtccttctccactgttccccggccctagcaagcctttctgggtcctggtggtggtgggcggagtgctggcctgctacagcctgctggtgaccgtggcctttatcatcttctgggtgaggagtaagaggagcaggctcctgcacagtgactacatgaacatgactcctagaagacctgggcctaccagaaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgctgataagtttaaactgccagaacattactctggcctaactggccggtaccggaggaaaaactgtttcatacagaaggcgtggaggaaaaactgtttcatacagaaggcgtggaggaaaaactgtttcatacagaaggcgtactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttgataa

SEQ ID NO: 120 - DNA, 3466 bp, synthetic construct
  source 1..3466: mol_type = other DNA
atggccttaccagtgaccgccttgctcctgccgctggccttgctgctccacgccgccaggccgcaggtgcagctggttgaatctggcggcggacttgtgaagcctggcggatctctgagactgagctgtgccgccagcggcttcacctttagcacatacgccatgagctggatcagacaggcccctggcaaaggcctggaatgggtggcgtctattggcagagccggcagcacctactacagcgactctgtgaagggcagattcaccatcagccgggacaacgccaagaacagcctgtacctgcagatgaactccctgagagccgaggacaccgccgtgtactattgtgccagaggacccatctacaacgactacgacgagttcgcctattggggccagggcacactggtcacagtttctagcggcggtggcggaagcggaggcggtggctccggtggcggaggcagcgaaattgtgctgacacagagccccgccacactgtcactttctccaggcgaaagagccacactgagctgcaagagcagccagagcgtgctgtactccagcaaccagaagaactacctggcctggtatcagcagaagcccggccaagctcctcggctgctgatctattgggccagcacaagagagagcggcatccctgccagattttctggcagcggctctggcaccgatttcaccctgaccataagcagcctggaacctgaggacttcgccgtgtattactgccaccagtacctgagcagcctgacctttggcggaggcaccaaggtggaaatcaagcggacaacaacccctgcccccagacctcctaccccagcccctacaattgccagccagcctctgagcctgaggcccgaggcttgtagacctgctgctggcggagccgtgcacaccagaggactggatttcgcctgcgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataagtttaaactgccagaacatttctctggcctaactggccggtaccggcttcattttttccatttactgcagaggcttcattttttccatttactgcagaggcttcattttttccatttactgcagaggcttcattttttccatttactgcagaggcttcattttttccatttactgcagaggcttcattttttccatttactgcagaactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttgataa

SEQ ID NO: 121 - DNA, 3385 bp, synthetic construct
  source 1..3385: mol_type = other DNA
atggccttaccagtgaccgccttgctcctgccgctggccttgctgctccacgccgccaggccgcaggtgcagctggttgaatctggcggcggacttgtgaagcctggcggatctctgagactgagctgtgccgccagcggcttcacctttagcacatacgccatgagctggatcagacaggcccctggcaaaggcctggaatgggtggcgtctattggcagagccggcagcacctactacagcgactctgtgaagggcagattcaccatcagccgggacaacgccaagaacagcctgtacctgcagatgaactccctgagagccgaggacaccgccgtgtactattgtgccagaggacccatctacaacgactacgacgagttcgcctattggggccagggcacactggtcacagtttctagcggcggtggcggaagcggaggcggtggctccggtggcggaggcagcgaaattgtgctgacacagagccccgccacactgtcactttctccaggcgaaagagccacactgagctgcaagagcagccagagcgtgctgtactccagcaaccagaagaactacctggcctggtatcagcagaagcccggccaagctcctcggctgctgatctattgggccagcacaagagagagcggcatccctgccagattttctggcagcggctctggcaccgatttcaccctgaccataagcagcctggaacctgaggacttcgccgtgtattactgccaccagtacctgagcagcctgacctttggcggaggcaccaaggtggaaatcaagcggacaacaacccctgcccccagacctcctaccccagcccctacaattgccagccagcctctgagcctgaggcccgaggcttgtagacctgctgctggcggagccgtgcacaccagaggactggatttcgcctgcgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataagtttaaactgccagaacatttctctggcctaactggccggtaccggcttcattttttccatttactgcagaggcttcattttttccatttactgcagaggcttcattttttccatttactgcagaactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttgataa

SEQ ID NO: 122 - DNA, 3484 bp, synthetic construct
  source 1..3484: mol_type = other DNA
atggccttaccagtgaccgccttgctcctgccgctggccttgctgctccacgccgccaggccgcaggtgcagctggttgaatctggcggcggacttgtgaagcctggcggatctctgagactgagctgtgccgccagcggcttcacctttagcacatacgccatgagctggatcagacaggcccctggcaaaggcctggaatgggtggcgtctattggcagagccggcagcacctactacagcgactctgtgaagggcagattcaccatcagccgggacaacgccaagaacagcctgtacctgcagatgaactccctgagagccgaggacaccgccgtgtactattgtgccagaggacccatctacaacgactacgacgagttcgcctattggggccagggcacactggtcacagtttctagcggcggtggcggaagcggaggcggtggctccggtggcggaggcagcgaaattgtgctgacacagagccccgccacactgtcactttctccaggcgaaagagccacactgagctgcaagagcagccagagcgtgctgtactccagcaaccagaagaactacctggcctggtatcagcagaagcccggccaagctcctcggctgctgatctattgggccagcacaagagagagcggcatccctgccagattttctggcagcggctctggcaccgatttcaccctgaccataagcagcctggaacctgaggacttcgccgtgtattactgccaccagtacctgagcagcctgacctttggcggaggcaccaaggtggaaatcaagcggacaacaacccctgcccccagacctcctaccccagcccctacaattgccagccagcctctgagcctgaggcccgaggcttgtagacctgctgctggcggagccgtgcacaccagaggactggatttcgcctgcgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataagtttaaactgccagaacatttctctggcctaactggccggtaccggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttgataa

SEQ ID NO: 123 - DNA, 3394 bp, synthetic construct
  source 1..3394: mol_type = other DNA
atggccttaccagtgaccgccttgctcctgccgctggccttgctgctccacgccgccaggccgcaggtgcagctggttgaatctggcggcggacttgtgaagcctggcggatctctgagactgagctgtgccgccagcggcttcacctttagcacatacgccatgagctggatcagacaggcccctggcaaaggcctggaatgggtggcgtctattggcagagccggcagcacctactacagcgactctgtgaagggcagattcaccatcagccgggacaacgccaagaacagcctgtacctgcagatgaactccctgagagccgaggacaccgccgtgtactattgtgccagaggacccatctacaacgactacgacgagttcgcctattggggccagggcacactggtcacagtttctagcggcggtggcggaagcggaggcggtggctccggtggcggaggcagcgaaattgtgctgacacagagccccgccacactgtcactttctccaggcgaaagagccacactgagctgcaagagcagccagagcgtgctgtactccagcaaccagaagaactacctggcctggtatcagcagaagcccggccaagctcctcggctgctgatctattgggccagcacaagagagagcggcatccctgccagattttctggcagcggctctggcaccgatttcaccctgaccataagcagcctggaacctgaggacttcgccgtgtattactgccaccagtacctgagcagcctgacctttggcggaggcaccaaggtggaaatcaagcggacaacaacccctgcccccagacctcctaccccagcccctacaattgccagccagcctctgagcctgaggcccgaggcttgtagacctgctgctggcggagccgtgcacaccagaggactggatttcgcctgcgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataagtttaaactgccagaacatttctctggcctaactggccggtaccggaggaaaaactgtttcatacagaaggcgtggaggaaaaactgtttcatacagaaggcgtggaggaaaaactgtttcatacagaaggcgtactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttgataa

SEQ ID NO: 124 - DNA, 3382 bp, synthetic construct
  source 1..3382: mol_type = other DNA
atggccttaccagtgaccgccttgctcctgccgctggccttgctgctccacgccgccaggccgcaggtgcagctggttgaatctggcggcggacttgtgaagcctggcggatctctgagactgagctgtgccgccagcggcttcacctttagcacatacgccatgagctggatcagacaggcccctggcaaaggcctggaatgggtggcgtctattggcagagccggcagcacctactacagcgactctgtgaagggcagattcaccatcagccgggacaacgccaagaacagcctgtacctgcagatgaactccctgagagccgaggacaccgccgtgtactattgtgccagaggacccatctacaacgactacgacgagttcgcctattggggccagggcacactggtcacagtttctagcggcggtggcggaagcggaggcggtggctccggtggcggaggcagcgaaattgtgctgacacagagccccgccacactgtcactttctccaggcgaaagagccacactgagctgcaagagcagccagagcgtgctgtactccagcaaccagaagaactacctggcctggtatcagcagaagcccggccaagctcctcggctgctgatctattgggccagcacaagagagagcggcatccctgccagattttctggcagcggctctggcaccgatttcaccctgaccataagcagcctggaacctgaggacttcgccgtgtattactgccaccagtacctgagcagcctgacctttggcggaggcaccaaggtggaaatcaagcggaagcacctgtgtccttctccactgttccccggccctagcaagcctttctgggtcctggtggtggtgggcggagtgctggcctgctacagcctgctggtgaccgtggcctttatcatcttctgggtgaggagtaagaggagcaggctcctgcacagtgactacatgaacatgactcctagaagacctgggcctaccagaaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataagtttaaactgccagaacatttctctggcctaactggccggtaccggcttcattttttccatttactgcagaggcttcattttttccatttactgcagaggcttcattttttccatttactgcagaggcttcattttttccatttactgcagaggcttcattttttccatttactgcagaggcttcattttttccatttactgcagaactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttgataa

SEQ ID NO: 125 - DNA, 3301 bp, synthetic construct
  source 1..3301: mol_type = other DNA
atggccttaccagtgaccgccttgctcctgccgctggccttgctgctccacgccgccaggccgcaggtgcagctggttgaatctggcggcggacttgtgaagcctggcggatctctgagactgagctgtgccgccagcggcttcacctttagcacatacgccatgagctggatcagacaggcccctggcaaaggcctggaatgggtggcgtctattggcagagccggcagcacctactacagcgactctgtgaagggcagattcaccatcagccgggacaacgccaagaacagcctgtacctgcagatgaactccctgagagccgaggacaccgccgtgtactattgtgccagaggacccatctacaacgactacgacgagttcgcctattggggccagggcacactggtcacagtttctagcggcggtggcggaagcggaggcggtggctccggtggcggaggcagcgaaattgtgctgacacagagccccgccacactgtcactttctccaggcgaaagagccacactgagctgcaagagcagccagagcgtgctgtactccagcaaccagaagaactacctggcctggtatcagcagaagcccggccaagctcctcggctgctgatctattgggccagcacaagagagagcggcatccctgccagattttctggcagcggctctggcaccgatttcaccctgaccataagcagcctggaacctgaggacttcgccgtgtattactgccaccagtacctgagcagcctgacctttggcggaggcaccaaggtggaaatcaagcggaagcacctgtgtccttctccactgttccccggccctagcaagcctttctgggtcctggtggtggtgggcggagtgctggcctgctacagcctgctggtgaccgtggcctttatcatcttctgggtgaggagtaagaggagcaggctcctgcacagtgactacatgaacatgactcctagaagacctgggcctaccagaaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataagtttaaactgccagaacatttctctggcctaactggccggtaccggcttcattttttccatttactgcagaggcttcattttttccatttactgcagaggcttcattttttccatttactgcagaactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttgataa

SEQ ID NO: 126 - DNA, 3400 bp, synthetic construct
  source 1..3400: mol_type = other DNA
atggccttaccagtgaccgccttgctcctgccgctggccttgctgctccacgccgccaggccgcaggtgcagctggttgaatctggcggcggacttgtgaagcctggcggatctctgagactgagctgtgccgccagcggcttcacctttagcacatacgccatgagctggatcagacaggcccctggcaaaggcctggaatgggtggcgtctattggcagagccggcagcacctactacagcgactctgtgaagggcagattcaccatcagccgggacaacgccaagaacagcctgtacctgcagatgaactccctgagagccgaggacaccgccgtgtactattgtgccagaggacccatctacaacgactacgacgagttcgcctattggggccagggcacactggtcacagtttctagcggcggtggcggaagcggaggcggtggctccggtggcggaggcagcgaaattgtgctgacacagagccccgccacactgtcactttctccaggcgaaagagccacactgagctgcaagagcagccagagcgtgctgtactccagcaaccagaagaactacctggcctggtatcagcagaagcccggccaagctcctcggctgctgatctattgggccagcacaagagagagcggcatccctgccagattttctggcagcggctctggcaccgatttcaccctgaccataagcagcctggaacctgaggacttcgccgtgtattactgccaccagtacctgagcagcctgacctttggcggaggcaccaaggtggaaatcaagcggaagcacctgtgtccttctccactgttccccggccctagcaagcctttctgggtcctggtggtggtgggcggagtgctggcctgctacagcctgctggtgaccgtggcctttatcatcttctgggtgaggagtaagaggagcaggctcctgcacagtgactacatgaacatgactcctagaagacctgggcctaccagaaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataagtttaaactgccagaacatttctctggcctaactggccggtaccggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttgataa

SEQ ID NO: 127 - DNA, 3310 bp, synthetic construct
  source 1..3310: mol_type = other DNA
atggccttaccagtgaccgccttgctcctgccgctggccttgctgctccacgccgccaggccgcaggtgcagctggttgaatctggcggcggacttgtgaagcctggcggatctctgagactgagctgtgccgccagcggcttcacctttagcacatacgccatgagctggatcagacaggcccctggcaaaggcctggaatgggtggcgtctattggcagagccggcagcacctactacagcgactctgtgaagggcagattcaccatcagccgggacaacgccaagaacagcctgtacctgcagatgaactccctgagagccgaggacaccgccgtgtactattgtgccagaggacccatctacaacgactacgacgagttcgcctattggggccagggcacactggtcacagtttctagcggcggtggcggaagcggaggcggtggctccggtggcggaggcagcgaaattgtgctgacacagagccccgccacactgtcactttctccaggcgaaagagccacactgagctgcaagagcagccagagcgtgctgtactccagcaaccagaagaactacctggcctggtatcagcagaagcccggccaagctcctcggctgctgatctattgggccagcacaagagagagcggcatccctgccagattttctggcagcggctctggcaccgatttcaccctgaccataagcagcctggaacctgaggacttcgccgtgtattactgccaccagtacctgagcagcctgacctttggcggaggcaccaaggtggaaatcaagcggaagcacctgtgtccttctccactgttccccggccctagcaagcctttctgggtcctggtggtggtgggcggagtgctggcctgctacagcctgctggtgaccgtggcctttatcatcttctgggtgaggagtaagaggagcaggctcctgcacagtgactacatgaacatgactcctagaagacctgggcctaccagaaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataagtttaaactgccagaacatttctctggcctaactggccggtaccggaggaaaaactgtttcatacagaaggcgtggaggaaaaactgtttcatacagaaggcgtggaggaaaaactgtttcatacagaaggcgtactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttgataa

SEQ ID NO: 128 - DNA, 3382 bp, synthetic construct
  source 1..3382: mol_type = other DNA
atggccttaccagtgaccgccttgctcctgccgctggccttgctgctccacgccgccaggccgcaggtgcagctggttgaatctggcggcggacttgtgaagcctggcggatctctgagactgagctgtgccgccagcggcttcacctttagcacatacgccatgagctggatcagacaggcccctggcaaaggcctggaatgggtggcgtctattggcagagccggcagcacctactacagcgactctgtgaagggcagattcaccatcagccgggacaacgccaagaacagcctgtacctgcagatgaactccctgagagccgaggacaccgccgtgtactattgtgccagaggacccatctacaacgactacgacgagttcgcctattggggccagggcacactggtcacagtttctagcggcggtggcggaagcggaggcggtggctccggtggcggaggcagcgaaattgtgctgacacagagccccgccacactgtcactttctccaggcgaaagagccacactgagctgcaagagcagccagagcgtgctgtactccagcaaccagaagaactacctggcctggtatcagcagaagcccggccaagctcctcggctgctgatctattgggccagcacaagagagagcggcatccctgccagattttctggcagcggctctggcaccgatttcaccctgaccataagcagcctggaacctgaggacttcgccgtgtattactgccaccagtacctgagcagcctgacctttggcggaggcaccaaggtggaaatcaagcggaagcacctgtgtccttctccactgttccccggccctagcaagcctttctgggtcctggtggtggtgggcggagtgctggcctgctacagcctgctggtgaccgtggcctttatcatcttctgggtgaggagtaagaggagcaggctcctgcacagtgactacatgaacatgactcctagaagacctgggcctaccagaaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgctgataagtttaaactgccagaacattactctggcctaactggccggtaccggcttcattttttccatttactgcagaggcttcattttttccatttactgcagaggcttcattttttccatttactgcagaggcttcattttttccatttactgcagaggcttcattttttccatttactgcagaggcttcattttttccatttactgcagaactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttgataa

SEQ ID NO: 129 - DNA, 3301 bp, synthetic construct
  source 1..3301: mol_type = other DNA
atggccttaccagtgaccgccttgctcctgccgctggccttgctgctccacgccgccaggccgcaggtgcagctggttgaatctggcggcggacttgtgaagcctggcggatctctgagactgagctgtgccgccagcggcttcacctttagcacatacgccatgagctggatcagacaggcccctggcaaaggcctggaatgggtggcgtctattggcagagccggcagcacctactacagcgactctgtgaagggcagattcaccatcagccgggacaacgccaagaacagcctgtacctgcagatgaactccctgagagccgaggacaccgccgtgtactattgtgccagaggacccatctacaacgactacgacgagttcgcctattggggccagggcacactggtcacagtttctagcggcggtggcggaagcggaggcggtggctccggtggcggaggcagcgaaattgtgctgacacagagccccgccacactgtcactttctccaggcgaaagagccacactgagctgcaagagcagccagagcgtgctgtactccagcaaccagaagaactacctggcctggtatcagcagaagcccggccaagctcctcggctgctgatctattgggccagcacaagagagagcggcatccctgccagattttctggcagcggctctggcaccgatttcaccctgaccataagcagcctggaacctgaggacttcgccgtgtattactgccaccagtacctgagcagcctgacctttggcggaggcaccaaggtggaaatcaagcggaagcacctgtgtccttctccactgttccccggccctagcaagcctttctgggtcctggtggtggtgggcggagtgctggcctgctacagcctgctggtgaccgtggcctttatcatcttctgggtgaggagtaagaggagcaggctcctgcacagtgactacatgaacatgactcctagaagacctgggcctaccagaaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgctgataagtttaaactgccagaacattactctggcctaactggccggtaccggcttcattttttccatttactgcagaggcttcattttttccatttactgcagaggcttcattttttccatttactgcagaactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttgataa

SEQ ID NO: 130 - DNA, 3400 bp, synthetic construct
  source 1..3400: mol_type = other DNA
atggccttaccagtgaccgccttgctcctgccgctggccttgctgctccacgccgccaggccgcaggtgcagctggttgaatctggcggcggacttgtgaagcctggcggatctctgagactgagctgtgccgccagcggcttcacctttagcacatacgccatgagctggatcagacaggcccctggcaaaggcctggaatgggtggcgtctattggcagagccggcagcacctactacagcgactctgtgaagggcagattcaccatcagccgggacaacgccaagaacagcctgtacctgcagatgaactccctgagagccgaggacaccgccgtgtactattgtgccagaggacccatctacaacgactacgacgagttcgcctattggggccagggcacactggtcacagtttctagcggcggtggcggaagcggaggcggtggctccggtggcggaggcagcgaaattgtgctgacacagagccccgccacactgtcactttctccaggcgaaagagccacactgagctgcaagagcagccagagcgtgctgtactccagcaaccagaagaactacctggcctggtatcagcagaagcccggccaagctcctcggctgctgatctattgggccagcacaagagagagcggcatccctgccagattttctggcagcggctctggcaccgatttcaccctgaccataagcagcctggaacctgaggacttcgccgtgtattactgccaccagtacctgagcagcctgacctttggcggaggcaccaaggtggaaatcaagcggaagcacctgtgtccttctccactgttccccggccctagcaagcctttctgggtcctggtggtggtgggcggagtgctggcctgctacagcctgctggtgaccgtggcctttatcatcttctgggtgaggagtaagaggagcaggctcctgcacagtgactacatgaacatgactcctagaagacctgggcctaccagaaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgctgataagtttaaactgccagaacattactctggcctaactggccggtaccggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttgataa

SEQ ID NO: 131 - DNA, 3310 bp, synthetic construct
  source 1..3310: mol_type = other DNA
atggccttaccagtgaccgccttgctcctgccgctggccttgctgctccacgccgccaggccgcaggtgcagctggttgaatctggcggcggacttgtgaagcctggcggatctctgagactgagctgtgccgccagcggcttcacctttagcacatacgccatgagctggatcagacaggcccctggcaaaggcctggaatgggtggcgtctattggcagagccggcagcacctactacagcgactctgtgaagggcagattcaccatcagccgggacaacgccaagaacagcctgtacctgcagatgaactccctgagagccgaggacaccgccgtgtactattgtgccagaggacccatctacaacgactacgacgagttcgcctattggggccagggcacactggtcacagtttctagcggcggtggcggaagcggaggcggtggctccggtggcggaggcagcgaaattgtgctgacacagagccccgccacactgtcactttctccaggcgaaagagccacactgagctgcaagagcagccagagcgtgctgtactccagcaaccagaagaactacctggcctggtatcagcagaagcccggccaagctcctcggctgctgatctattgggccagcacaagagagagcggcatccctgccagattttctggcagcggctctggcaccgatttcaccctgaccataagcagcctggaacctgaggacttcgccgtgtattactgccaccagtacctgagcagcctgacctttggcggaggcaccaaggtggaaatcaagcggaagcacctgtgtccttctccactgttccccggccctagcaagcctttctgggtcctggtggtggtgggcggagtgctggcctgctacagcctgctggtgaccgtggcctttatcatcttctgggtgaggagtaagaggagcaggctcctgcacagtgactacatgaacatgactcctagaagacctgggcctaccagaaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgctgataagtttaaactgccagaacattactctggcctaactggccggtaccggaggaaaaactgtttcatacagaaggcgtggaggaaaaactgtttcatacagaaggcgtggaggaaaaactgtttcatacagaaggcgtactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttgataa

SEQ ID NO: 132 - DNA, 3947 bp, synthetic construct
  source 1..3947: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtggctatgccatgagctgggtccgccaggctccagggaaggggctggagtgggtctcaaccattagtagtggcggaacctacatatactaccccgactcagtgaagggccgattcaccatctccagagacaacgccaagaactcactgtatctgcaaatgaacagcctgagagccgaggacacggccgtgtattactgtgcgagacttgggggggataattactacgaatacttcgatgtctggggcaaagggaccacggtcaccgtctcctccggcggtggcggatccggcggtggcggatccggcggtggcggatccgacattgtgctgacccagtctccagcctccttggccgtgtctccaggacagagggccaccatcacctgcagagccagtaagagtgtcagtaccagcggatactcctacatgcactggtatcagcagaaaccaggacaacctcctaaactcctgatttacctggcatccaatctggagagcggggtcccagccaggttcagcggcagtgggtctgggaccgatttcaccctcacaattaatcctgtggaagctaatgatactgcaaattattactgtcagcacagtagggagctgcctttcacattcggcggagggaccaaggtggagatcaaacgaactacaacaacccctgcccccagacctcctaccccagcccctacaattgccagccagcctctgagcctgaggcccgaggcttgtagacctgctgctggcggagccgtgcacaccagaggactggatttcgcctgcgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataagtttaaactgccagaacatttctctggcctaactggccggtaccggcttcattttttccatttactgcagaggcttcattttttccatttactgcagaggcttcattttttccatttactgcagaggcttcattttttccatttactgcagaggcttcattttttccatttactgcagaggcttcattttttccatttactgcagaactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtctggaagcggtgagggaagaggtagcctgctcacgtgcggtgatgtggaggagaaccctgggcctatgctctgtttgggctggatatttctttggcttgttgcaggagagcgaattaaaggatttaatatttcaggttgttccacaaaaaaactcctttggacatattctacaaggagtgaagaggaatttgtcttattttgtgatttaccagagccacagaaatcacatttctgccacagaaatcgactctcaccaaaacaagtccctgagcacctgcccttcatgggtagtaacgacctatctgatgtccaatggtaccaacaaccttcgaatggagatccattagaggacattaggaaaagctatcctcacatcattcaggacaaatgtacccttcactttttgaccccaggggtgaataattctgggtcatatatttgtagacccaagatgattaagagcccctatgatgtagcctgttgtg

SEQ ID NO: 133 - DNA, 3947 bp, synthetic construct
  source 1..3947: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtggctatgccatgagctgggtccgccaggctccagggaaggggctggagtgggtctcaaccattagtagtggcggaacctacatatactaccccgactcagtgaagggccgattcaccatctccagagacaacgccaagaactcactgtatctgcaaatgaacagcctgagagccgaggacacggccgtgtattactgtgcgagacttgggggggataattactacgaatacttcgatgtctggggcaaagggaccacggtcaccgtctcctccggcggtggcggatccggcggtggcggatccggcggtggcggatccgacattgtgctgacccagtctccagcctccttggccgtgtctccaggacagagggccaccatcacctgcagagccagtaagagtgtcagtaccagcggatactcctacatgcactggtatcagcagaaaccaggacaacctcctaaactcctgatttacctggcatccaatctggagagcggggtcccagccaggttcagcggcagtgggtctgggaccgatttcaccctcacaattaatcctgtggaagctaatgatactgcaaattattactgtcagcacagtagggagctgcctttcacattcggcggagggaccaaggtggagatcaaacgaactacaacaacccctgcccccagacctcctaccccagcccctacaattgccagccagcctctgagcctgaggcccgaggcttgtagacctgctgctggcggagccgtgcacaccagaggactggatttcgcctgcgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataagtttaaactgccagaacatttctctggcctaactggccggtaccggcttcattttttccatttactgcagaggcttcattttttccatttactgcagaggcttcattttttccatttactgcagaactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtctggaagcggtgagggaagaggtagcctgctcacgtgcggtgatgtggaggagaaccctgggcctatgctctgtttgggctggatatttctttggcttgttgcaggagagcgaattaaaggatttaatatttcaggttgttccacaaaaaaactcctttggacatattctacaaggagtgaagaggaatttgtcttattttgtgatttaccagagccacagaaatcacatttctgccacagaaatcgactctcaccaaaacaagtccctgagcacctgcccttcatgggtagtaacgacctatctgatgtccaatggtaccaacaaccttcgaatggagatccattagaggacattaggaaaagctatcctcacatcattcaggacaaatgtacccttcactttttgaccccaggggtgaataattctgggtcatatatttgtagacccaagatgattaagagcccctatgatgtagcctgttgtgtcaagatgattttagaagttaagccccagacaaatgcatcctgtgagtattccgcatcacataagcaagacctacttcttg

SEQ ID NO: 134 - DNA, 3947 bp, synthetic construct
  source 1..3947: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtggctatgccatgagctgggtccgccaggctccagggaaggggctggagtgggtctcaaccattagtagtggcggaacctacatatactaccccgactcagtgaagggccgattcaccatctccagagacaacgccaagaactcactgtatctgcaaatgaacagcctgagagccgaggacacggccgtgtattactgtgcgagacttgggggggataattactacgaatacttcgatgtctggggcaaagggaccacggtcaccgtctcctccggcggtggcggatccggcggtggcggatccggcggtggcggatccgacattgtgctgacccagtctccagcctccttggccgtgtctccaggacagagggccaccatcacctgcagagccagtaagagtgtcagtaccagcggatactcctacatgcactggtatcagcagaaaccaggacaacctcctaaactcctgatttacctggcatccaatctggagagcggggtcccagccaggttcagcggcagtgggtctgggaccgatttcaccctcacaattaatcctgtggaagctaatgatactgcaaattattactgtcagcacagtagggagctgcctttcacattcggcggagggaccaaggtggagatcaaacgaactacaacaacccctgcccccagacctcctaccccagcccctacaattgccagccagcctctgagcctgaggcccgaggcttgtagacctgctgctggcggagccgtgcacaccagaggactggatttcgcctgcgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataagtttaaactgccagaacatttctctggcctaactggccggtaccggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtctggaagcggtgagggaagaggtagcctgctcacgtgcggtgatgtggaggagaaccctgggcctatgctctgtttgggctggatatttctttggcttgttgcaggagagcgaattaaaggatttaatatttcaggttgttccacaaaaaaactcctttggacatattctacaaggagtgaagaggaatttgtcttattttgtgatttaccagagccacagaaatcacatttctgccacagaaatcgactctcaccaaaacaagtccctgagcacctgcccttcatgggtagtaacgacctatctgatgtccaatggtaccaacaaccttcgaatggagatccattagaggacattaggaaaagctatcctcacatcattcaggacaaatgtacccttcactttttgaccccaggggtgaataattctgggtcatatatttgtagacccaagatgattaagagcccct

SEQ ID NO: 135 - DNA, 3947 bp, synthetic construct
  source 1..3947: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtggctatgccatgagctgggtccgccaggctccagggaaggggctggagtgggtctcaaccattagtagtggcggaacctacatatactaccccgactcagtgaagggccgattcaccatctccagagacaacgccaagaactcactgtatctgcaaatgaacagcctgagagccgaggacacggccgtgtattactgtgcgagacttgggggggataattactacgaatacttcgatgtctggggcaaagggaccacggtcaccgtctcctccggcggtggcggatccggcggtggcggatccggcggtggcggatccgacattgtgctgacccagtctccagcctccttggccgtgtctccaggacagagggccaccatcacctgcagagccagtaagagtgtcagtaccagcggatactcctacatgcactggtatcagcagaaaccaggacaacctcctaaactcctgatttacctggcatccaatctggagagcggggtcccagccaggttcagcggcagtgggtctgggaccgatttcaccctcacaattaatcctgtggaagctaatgatactgcaaattattactgtcagcacagtagggagctgcctttcacattcggcggagggaccaaggtggagatcaaacgaactacaacaacccctgcccccagacctcctaccccagcccctacaattgccagccagcctctgagcctgaggcccgaggcttgtagacctgctgctggcggagccgtgcacaccagaggactggatttcgcctgcgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataagtttaaactgccagaacatttctctggcctaactggccggtaccggaggaaaaactgtttcatacagaaggcgtggaggaaaaactgtttcatacagaaggcgtggaggaaaaactgtttcatacagaaggcgtactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtctggaagcggtgagggaagaggtagcctgctcacgtgcggtgatgtggaggagaaccctgggcctatgctctgtttgggctggatatttctttggcttgttgcaggagagcgaattaaaggatttaatatttcaggttgttccacaaaaaaactcctttggacatattctacaaggagtgaagaggaatttgtcttattttgtgatttaccagagccacagaaatcacatttctgccacagaaatcgactctcaccaaaacaagtccctgagcacctgcccttcatgggtagtaacgacctatctgatgtccaatggtaccaacaaccttcgaatggagatccattagaggacattaggaaaagctatcctcacatcattcaggacaaatgtacccttcactttttgaccccaggggtgaataattctgggtcatatatttgtagacccaagatgattaagagcccctatgatgtagcctgttgtgtcaagatgattttagaagttaagccccagacaaatgcatcctgtgagtattccgcatcacataagcaagacc

SEQ ID NO: 136 - DNA, 3947 bp, synthetic construct
  source 1..3947: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtggctatgccatgagctgggtccgccaggctccagggaaggggctggagtgggtctcaaccattagtagtggcggaacctacatatactaccccgactcagtgaagggccgattcaccatctccagagacaacgccaagaactcactgtatctgcaaatgaacagcctgagagccgaggacacggccgtgtattactgtgcgagacttgggggggataattactacgaatacttcgatgtctggggcaaagggaccacggtcaccgtctcctccggcggtggcggatccggcggtggcggatccggcggtggcggatccgacattgtgctgacccagtctccagcctccttggccgtgtctccaggacagagggccaccatcacctgcagagccagtaagagtgtcagtaccagcggatactcctacatgcactggtatcagcagaaaccaggacaacctcctaaactcctgatttacctggcatccaatctggagagcggggtcccagccaggttcagcggcagtgggtctgggaccgatttcaccctcacaattaatcctgtggaagctaatgatactgcaaattattactgtcagcacagtagggagctgcctttcacattcggcggagggaccaaggtggagatcaaacgaactaagcacctgtgtccttctccactgttccccggccctagcaagcctttctgggtcctggtggtggtgggcggagtgctggcctgctacagcctgctggtgaccgtggcctttatcatcttctgggtgaggagtaagaggagcaggctcctgcacagtgactacatgaacatgactcctagaagacctgggcctaccagaaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataagtttaaactgccagaacatttctctggcctaactggccggtaccggcttcattttttccatttactgcagaggcttcattttttccatttactgcagaggcttcattttttccatttactgcagaggcttcattttttccatttactgcagaggcttcattttttccatttactgcagaggcttcattttttccatttactgcagaactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtctggaagcggtgagggaagaggtagcctgctcacgtgcggtgatgtggaggagaaccctgggcctatgctctgtttgggctggatatttctttggcttgttgcaggagagcgaattaaaggatttaatatttcaggttgttccacaaaaaaactcctttggacatattctacaaggagtgaagaggaatttgtcttattttgtgatttaccagagccacagaaatcacatttctgccacagaaatcgactctcaccaaaacaagtccctgagcacctgcccttcatgggtagtaacgacctatctgatgtccaatggtaccaacaaccttcgaatggagatccattagaggacattaggaaaagctatcctcacatcattcaggacaaatgtacccttcactttttgaccccaggggtgaataattctgggtcatatatttgtagacccaagatgattaagagcccctatgatgtagcctgttgtgtcaagatgattttagaagttaagccccagacaaatgcatcctgtgagtattccgcatcacataagcaagacctacttcttggga

SEQ ID NO: 137 - DNA, 3947 bp, synthetic construct
  source 1..3947: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtggctatgccatgagctgggtccgccaggctccagggaaggggctggagtgggtctcaaccattagtagtggcggaacctacatatactaccccgactcagtgaagggccgattcaccatctccagagacaacgccaagaactcactgtatctgcaaatgaacagcctgagagccgaggacacggccgtgtattactgtgcgagacttgggggggataattactacgaatacttcgatgtctggggcaaagggaccacggtcaccgtctcctccggcggtggcggatccggcggtggcggatccggcggtggcggatccgacattgtgctgacccagtctccagcctccttggccgtgtctccaggacagagggccaccatcacctgcagagccagtaagagtgtcagtaccagcggatactcctacatgcactggtatcagcagaaaccaggacaacctcctaaactcctgatttacctggcatccaatctggagagcggggtcccagccaggttcagcggcagtgggtctgggaccgatttcaccctcacaattaatcctgtggaagctaatgatactgcaaattattactgtcagcacagtagggagctgcctttcacattcggcggagggaccaaggtggagatcaaacgaactaagcacctgtgtccttctccactgttccccggccctagcaagcctttctgggtcctggtggtggtgggcggagtgctggcctgctacagcctgctggtgaccgtggcctttatcatcttctgggtgaggagtaagaggagcaggctcctgcacagtgactacatgaacatgactcctagaagacctgggcctaccagaaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataagtttaaactgccagaacatttctctggcctaactggccggtaccggcttcattttttccatttactgcagaggcttcattttttccatttactgcagaggcttcattttttccatttactgcagaactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtctggaagcggtgagggaagaggtagcctgctcacgtgcggtgatgtggaggagaaccctgggcctatgctctgtttgggctggatatttctttggcttgttgcaggagagcgaattaaaggatttaatatttcaggttgttccacaaaaaaactcctttggacatattctacaaggagtgaagaggaatttgtcttattttgtgatttaccagagccacagaaatcacatttctgccacagaaatcgactctcaccaaaacaagtccctgagcacctgcccttcatgggtagtaacgacctatctgatgtccaatggtaccaacaaccttcgaatggagatccattagaggacattaggaaaagctatcctcacatcattcaggacaaatgtacccttcactttttgaccccaggggtgaataattctgggtcatatatttgtagacccaagatgattaagagcccctatgatgtagcctgttgtgtcaagatgattttagaagttaagccccagacaaatgcatcctgtgagtattccgcatcacataagcaagacctacttcttgggagcactggctctatttcttgccccagtctcagctgccaaagtgatgcacaaagtccagcggtaacctggtacaagaatggaa

SEQ ID NO: 138 - DNA, 3947 bp, synthetic construct
  source 1..3947: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtggctatgccatgagctgggtccgccaggctccagggaaggggctggagtgggtctcaaccattagtagtggcggaacctacatatactaccccgactcagtgaagggccgattcaccatctccagagacaacgccaagaactcactgtatctgcaaatgaacagcctgagagccgaggacacggccgtgtattactgtgcgagacttgggggggataattactacgaatacttcgatgtctggggcaaagggaccacggtcaccgtctcctccggcggtggcggatccggcggtggcggatccggcggtggcggatccgacattgtgctgacccagtctccagcctccttggccgtgtctccaggacagagggccaccatcacctgcagagccagtaagagtgtcagtaccagcggatactcctacatgcactggtatcagcagaaaccaggacaacctcctaaactcctgatttacctggcatccaatctggagagcggggtcccagccaggttcagcggcagtgggtctgggaccgatttcaccctcacaattaatcctgtggaagctaatgatactgcaaattattactgtcagcacagtagggagctgcctttcacattcggcggagggaccaaggtggagatcaaacgaactaagcacctgtgtccttctccactgttccccggccctagcaagcctttctgggtcctggtggtggtgggcggagtgctggcctgctacagcctgctggtgaccgtggcctttatcatcttctgggtgaggagtaagaggagcaggctcctgcacagtgactacatgaacatgactcctagaagacctgggcctaccagaaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataagtttaaactgccagaacatttctctggcctaactggccggtaccggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtctggaagcggtgagggaagaggtagcctgctcacgtgcggtgatgtggaggagaaccctgggcctatgctctgtttgggctggatatttctttggcttgttgcaggagagcgaattaaaggatttaatatttcaggttgttccacaaaaaaactcctttggacatattctacaaggagtgaagaggaatttgtcttattttgtgatttaccagagccacagaaatcacatttctgccacagaaatcgactctcaccaaaacaagtccctgagcacctgcccttcatgggtagtaacgacctatctgatgtccaatggtaccaacaaccttcgaatggagatccattagaggacattaggaaaagctatcctcacatcattcaggacaaatgtacccttcactttttgaccccaggggtgaataattctgggtcatatatttgtagacccaagatgattaagagcccctatgatgtagcctgttgtgtcaagatgattttagaagttaagccccagacaaatgcatcctgtgagtattccgcatcacataagc

SEQ ID NO: 139 - DNA, 3947 bp, synthetic construct
  source 1..3947: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtggctatgccatgagctgggtccgccaggctccagggaaggggctggagtgggtctcaaccattagtagtggcggaacctacatatactaccccgactcagtgaagggccgattcaccatctccagagacaacgccaagaactcactgtatctgcaaatgaacagcctgagagccgaggacacggccgtgtattactgtgcgagacttgggggggataattactacgaatacttcgatgtctggggcaaagggaccacggtcaccgtctcctccggcggtggcggatccggcggtggcggatccggcggtggcggatccgacattgtgctgacccagtctccagcctccttggccgtgtctccaggacagagggccaccatcacctgcagagccagtaagagtgtcagtaccagcggatactcctacatgcactggtatcagcagaaaccaggacaacctcctaaactcctgatttacctggcatccaatctggagagcggggtcccagccaggttcagcggcagtgggtctgggaccgatttcaccctcacaattaatcctgtggaagctaatgatactgcaaattattactgtcagcacagtagggagctgcctttcacattcggcggagggaccaaggtggagatcaaacgaactaagcacctgtgtccttctccactgttccccggccctagcaagcctttctgggtcctggtggtggtgggcggagtgctggcctgctacagcctgctggtgaccgtggcctttatcatcttctgggtgaggagtaagaggagcaggctcctgcacagtgactacatgaacatgactcctagaagacctgggcctaccagaaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataagtttaaactgccagaacatttctctggcctaactggccggtaccggaggaaaaactgtttcatacagaaggcgtggaggaaaaactgtttcatacagaaggcgtggaggaaaaactgtttcatacagaaggcgtactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtctggaagcggtgagggaagaggtagcctgctcacgtgcggtgatgtggaggagaaccctgggcctatgctctgtttgggctggatatttctttggcttgttgcaggagagcgaattaaaggatttaatatttcaggttgttccacaaaaaaactcctttggacatattctacaaggagtgaagaggaatttgtcttattttgtgatttaccagagccacagaaatcacatttctgccacagaaatcgactctcaccaaaacaagtccctgagcacctgcccttcatgggtagtaacgacctatctgatgtccaatggtaccaacaaccttcgaatggagatccattagaggacattaggaaaagctatcctcacatcattcaggacaaatgtacccttcactttttgaccccaggggtgaataattctgggtcatatatttgtagacccaagatgattaagagcccctatgatgtagcctgttgtgtcaagatgattttagaagttaagccccagacaaatgcatcctgtgagtattccgcatcacataagcaagacctacttcttgggagcactggctctatttcttgccccagtctcagctgccaaagtgatgcacaaagtccagcggtaacctggtaca

SEQ ID NO: 140 - DNA, 3947 bp, synthetic construct
  source 1..3947: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtggctatgccatgagctgggtccgccaggctccagggaaggggctggagtgggtctcaaccattagtagtggcggaacctacatatactaccccgactcagtgaagggccgattcaccatctccagagacaacgccaagaactcactgtatctgcaaatgaacagcctgagagccgaggacacggccgtgtattactgtgcgagacttgggggggataattactacgaatacttcgatgtctggggcaaagggaccacggtcaccgtctcctccggcggtggcggatccggcggtggcggatccggcggtggcggatccgacattgtgctgacccagtctccagcctccttggccgtgtctccaggacagagggccaccatcacctgcagagccagtaagagtgtcagtaccagcggatactcctacatgcactggtatcagcagaaaccaggacaacctcctaaactcctgatttacctggcatccaatctggagagcggggtcccagccaggttcagcggcagtgggtctgggaccgatttcaccctcacaattaatcctgtggaagctaatgatactgcaaattattactgtcagcacagtagggagctgcctttcacattcggcggagggaccaaggtggagatcaaacgaactaagcacctgtgtccttctccactgttccccggccctagcaagcctttctgggtcctggtggtggtgggcggagtgctggcctgctacagcctgctggtgaccgtggcctttatcatcttctgggtgaggagtaagaggagcaggctcctgcacagtgactacatgaacatgactcctagaagacctgggcctaccagaaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgctgataagtttaaactgccagaacattactctggcctaactggccggtaccggcttcattttttccatttactgcagaggcttcattttttccatttactgcagaggcttcattttttccatttactgcagaggcttcattttttccatttactgcagaggcttcattttttccatttactgcagaggcttcattttttccatttactgcagaactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtctggaagcggtgagggaagaggtagcctgctcacgtgcggtgatgtggaggagaaccctgggcctatgctctgtttgggctggatatttctttggcttgttgcaggagagcgaattaaaggatttaatatttcaggttgttccacaaaaaaactcctttggacatattctacaaggagtgaagaggaatttgtcttattttgtgatttaccagagccacagaaatcacatttctgccacagaaatcgactctcaccaaaacaagtccctgagcacctgcccttcatgggtagtaacgacctatctgatgtccaatggtaccaacaaccttcgaatggagatccattagaggacattaggaaaagctatcctcacatcattcaggacaaatgtacccttcactttttgaccccaggggtgaataattctgggtcatatatttgtagacccaagatgattaagagcccctatgatgtagcctgttgtgtcaagatgattttagaagttaagccccagacaaatgcatcctgtgagtattccgcatcacataagcaagacctacttcttggga

SEQ ID NO: 141 - DNA, 3947 bp, synthetic construct
  source 1..3947: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtggctatgccatgagctgggtccgccaggctccagggaaggggctggagtgggtctcaaccattagtagtggcggaacctacatatactaccccgactcagtgaagggccgattcaccatctccagagacaacgccaagaactcactgtatctgcaaatgaacagcctgagagccgaggacacggccgtgtattactgtgcgagacttgggggggataattactacgaatacttcgatgtctggggcaaagggaccacggtcaccgtctcctccggcggtggcggatccggcggtggcggatccggcggtggcggatccgacattgtgctgacccagtctccagcctccttggccgtgtctccaggacagagggccaccatcacctgcagagccagtaagagtgtcagtaccagcggatactcctacatgcactggtatcagcagaaaccaggacaacctcctaaactcctgatttacctggcatccaatctggagagcggggtcccagccaggttcagcggcagtgggtctgggaccgatttcaccctcacaattaatcctgtggaagctaatgatactgcaaattattactgtcagcacagtagggagctgcctttcacattcggcggagggaccaaggtggagatcaaacgaactaagcacctgtgtccttctccactgttccccggccctagcaagcctttctgggtcctggtggtggtgggcggagtgctggcctgctacagcctgctggtgaccgtggcctttatcatcttctgggtgaggagtaagaggagcaggctcctgcacagtgactacatgaacatgactcctagaagacctgggcctaccagaaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgctgataagtttaaactgccagaacattactctggcctaactggccggtaccggcttcattttttccatttactgcagaggcttcattttttccatttactgcagaggcttcattttttccatttactgcagaactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtctggaagcggtgagggaagaggtagcctgctcacgtgcggtgatgtggaggagaaccctgggcctatgctctgtttgggctggatatttctttggcttgttgcaggagagcgaattaaaggatttaatatttcaggttgttccacaaaaaaactcctttggacatattctacaaggagtgaagaggaatttgtcttattttgtgatttaccagagccacagaaatcacatttctgccacagaaatcgactctcaccaaaacaagtccctgagcacctgcccttcatgggtagtaacgacctatctgatgtccaatggtaccaacaaccttcgaatggagatccattagaggacattaggaaaagctatcctcacatcattcaggacaaatgtacccttcactttttgaccccaggggtgaataattctgggtcatatatttgtagacccaagatgattaagagcccctatgatgtagcctgttgtgtcaagatgattttagaagttaagccccagacaaatgcatcctgtgagtattccgcatcacataagcaagacctacttcttgggagcactggctctatttcttgccccagtctcagctgccaaagtgatgcacaaagtccagcggtaacctggtacaagaatggaa

SEQ ID NO: 142 - DNA, 3947 bp, synthetic construct
  source 1..3947: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtggctatgccatgagctgggtccgccaggctccagggaaggggctggagtgggtctcaaccattagtagtggcggaacctacatatactaccccgactcagtgaagggccgattcaccatctccagagacaacgccaagaactcactgtatctgcaaatgaacagcctgagagccgaggacacggccgtgtattactgtgcgagacttgggggggataattactacgaatacttcgatgtctggggcaaagggaccacggtcaccgtctcctccggcggtggcggatccggcggtggcggatccggcggtggcggatccgacattgtgctgacccagtctccagcctccttggccgtgtctccaggacagagggccaccatcacctgcagagccagtaagagtgtcagtaccagcggatactcctacatgcactggtatcagcagaaaccaggacaacctcctaaactcctgatttacctggcatccaatctggagagcggggtcccagccaggttcagcggcagtgggtctgggaccgatttcaccctcacaattaatcctgtggaagctaatgatactgcaaattattactgtcagcacagtagggagctgcctttcacattcggcggagggaccaaggtggagatcaaacgaactaagcacctgtgtccttctccactgttccccggccctagcaagcctttctgggtcctggtggtggtgggcggagtgctggcctgctacagcctgctggtgaccgtggcctttatcatcttctgggtgaggagtaagaggagcaggctcctgcacagtgactacatgaacatgactcctagaagacctgggcctaccagaaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgctgataagtttaaactgccagaacattactctggcctaactggccggtaccggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtctggaagcggtgagggaagaggtagcctgctcacgtgcggtgatgtggaggagaaccctgggcctatgctctgtttgggctggatatttctttggcttgttgcaggagagcgaattaaaggatttaatatttcaggttgttccacaaaaaaactcctttggacatattctacaaggagtgaagaggaatttgtcttattttgtgatttaccagagccacagaaatcacatttctgccacagaaatcgactctcaccaaaacaagtccctgagcacctgcccttcatgggtagtaacgacctatctgatgtccaatggtaccaacaaccttcgaatggagatccattagaggacattaggaaaagctatcctcacatcattcaggacaaatgtacccttcactttttgaccccaggggtgaataattctgggtcatatatttgtagacccaagatgattaagagcccctatgatgtagcctgttgtgtcaagatgattttagaagttaagccccagacaaatgcatcctgtgagtattccgcatcacataagc

SEQ ID NO: 143 - DNA, 3947 bp, synthetic construct
  source 1..3947: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtggctatgccatgagctgggtccgccaggctccagggaaggggctggagtgggtctcaaccattagtagtggcggaacctacatatactaccccgactcagtgaagggccgattcaccatctccagagacaacgccaagaactcactgtatctgcaaatgaacagcctgagagccgaggacacggccgtgtattactgtgcgagacttgggggggataattactacgaatacttcgatgtctggggcaaagggaccacggtcaccgtctcctccggcggtggcggatccggcggtggcggatccggcggtggcggatccgacattgtgctgacccagtctccagcctccttggccgtgtctccaggacagagggccaccatcacctgcagagccagtaagagtgtcagtaccagcggatactcctacatgcactggtatcagcagaaaccaggacaacctcctaaactcctgatttacctggcatccaatctggagagcggggtcccagccaggttcagcggcagtgggtctgggaccgatttcaccctcacaattaatcctgtggaagctaatgatactgcaaattattactgtcagcacagtagggagctgcctttcacattcggcggagggaccaaggtggagatcaaacgaactaagcacctgtgtccttctccactgttccccggccctagcaagcctttctgggtcctggtggtggtgggcggagtgctggcctgctacagcctgctggtgaccgtggcctttatcatcttctgggtgaggagtaagaggagcaggctcctgcacagtgactacatgaacatgactcctagaagacctgggcctaccagaaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgctgataagtttaaactgccagaacattactctggcctaactggccggtaccggaggaaaaactgtttcatacagaaggcgtggaggaaaaactgtttcatacagaaggcgtggaggaaaaactgtttcatacagaaggcgtactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtctggaagcggtgagggaagaggtagcctgctcacgtgcggtgatgtggaggagaaccctgggcctatgctctgtttgggctggatatttctttggcttgttgcaggagagcgaattaaaggatttaatatttcaggttgttccacaaaaaaactcctttggacatattctacaaggagtgaagaggaatttgtcttattttgtgatttaccagagccacagaaatcacatttctgccacagaaatcgactctcaccaaaacaagtccctgagcacctgcccttcatgggtagtaacgacctatctgatgtccaatggtaccaacaaccttcgaatggagatccattagaggacattaggaaaagctatcctcacatcattcaggacaaatgtacccttcactttttgaccccaggggtgaataattctgggtcatatatttgtagacccaagatgattaagagcccctatgatgtagcctgttgtgtcaagatgattttagaagttaagccccagacaaatgcatcctgtgagtattccgcatcacataagcaagacctacttcttgggagcactggctctatttcttgccccagtctcagctgccaaagtgatgcacaaagtccagcggtaacctggtaca

SEQ ID NO: 144 - DNA, 3947 bp, synthetic construct; identical to SEQ ID NO: 108
  source 1..3947: mol_type = other DNA

SEQ ID NO: 145 - DNA, 3947 bp, synthetic construct
  source 1..3947: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtaggtatggcatgagctgggtccgccaggctccagggaagaggctggagtgggtctcaaccattagtggcggaggcacctacatatactacccagactcagtgaagggccgattcaccatctccagagacaacgccaagaacaccctgtatctgcaaatgaacagcctgagagccgaggacacggctgtgtattactgtaccagagataactatggccgcaactatgattatggcatggattattggggccagggcaccctggtgaccgtgagcagcggtggtggcggttccggcggtggcggttccggtggtggcggttccgaaattgtgttgacacagtctccagccaccctgtctttgtctccaggggaaagagccaccctcacctgcagcgccaccagcagtgttagctacatccactggtatcaacagaggcctggccagagccccaggctcctcatctatagcacctccaacctggccagcggcatcccagccaggttcagtggcagtgggtctgggagcgactacactctcaccatcagcagcctagagcctgaagattttgcagtttattactgtcagcagcgtagcagctcccctttcacctttggcagcggcaccaaagtggaaattaaaacaacaacccctgcccccagacctcctaccccagcccctacaattgccagccagcctctgagcctgaggcccgaggcttgtagacctgctgctggcggagccgtgcacaccagaggactggatttcgcctgcgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataagtttaaactgccagaacatttctctggcctaactggccggtaccggcttcattttttccatttactgcagaggcttcattttttccatttactgcagaggcttcattttttccatttactgcagaactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtctggaagcggtgagggaagaggtagcctgctcacgtgcggtgatgtggaggagaaccctgggcctatgctctgtttgggctggatatttctttggcttgttgcaggagagcgaattaaaggatttaatatttcaggttgttccacaaaaaaactcctttggacatattctacaaggagtgaagaggaatttgtcttattttgtgatttaccagagccacagaaatcacatttctgccacagaaatcgactctcaccaaaacaagtccctgagcacctgcccttcatgggtagtaacgacctatctgatgtccaatggtaccaacaaccttcgaatggagatccattagaggacattaggaaaagctatcctcacatcattcaggacaaatgtacccttcactttttgaccccaggggtgaataattctgggtcatatatttgtagacccaagatgattaagagcccctatgatgtagcctgttgtgtcaagatgattttagaagttaagccccagacaaatgcatcctgtgagtattccgcatcacataagcaagacctacttcttgggagcactggctctattt

SEQ ID NO: 146 - DNA, 3947 bp, synthetic construct
  source 1..3947: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtaggtatggcatgagctgggtccgccaggctccagggaagaggctggagtgggtctcaaccattagtggcggaggcacctacatatactacccagactcagtgaagggccgattcaccatctccagagacaacgccaagaacaccctgtatctgcaaatgaacagcctgagagccgaggacacggctgtgtattactgtaccagagataactatggccgcaactatgattatggcatggattattggggccagggcaccctggtgaccgtgagcagcggtggtggcggttccggcggtggcggttccggtggtggcggttccgaaattgtgttgacacagtctccagccaccctgtctttgtctccaggggaaagagccaccctcacctgcagcgccaccagcagtgttagctacatccactggtatcaacagaggcctggccagagccccaggctcctcatctatagcacctccaacctggccagcggcatcccagccaggttcagtggcagtgggtctgggagcgactacactctcaccatcagcagcctagagcctgaagattttgcagtttattactgtcagcagcgtagcagctcccctttcacctttggcagcggcaccaaagtggaaattaaaacaacaacccctgcccccagacctcctaccccagcccctacaattgccagccagcctctgagcctgaggcccgaggcttgtagacctgctgctggcggagccgtgcacaccagaggactggatttcgcctgcgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataagtttaaactgccagaacatttctctggcctaactggccggtaccggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtctggaagcggtgagggaagaggtagcctgctcacgtgcggtgatgtggaggagaaccctgggcctatgctctgtttgggctggatatttctttggcttgttgcaggagagcgaattaaaggatttaatatttcaggttgttccacaaaaaaactcctttggacatattctacaaggagtgaagaggaatttgtcttattttgtgatttaccagagccacagaaatcacatttctgccacagaaatcgactctcaccaaaacaagtccctgagcacctgcccttcatgggtagtaacgacctatctgatgtccaatggtaccaacaaccttcgaatggagatccattagaggacattaggaaaagctatcctcacatcattcaggacaaatgtacccttcactttttgaccccaggggtgaataattctgggtcatatatttgtagacccaagatgattaagagcccctatgatgtagcctgttgtg

SEQ ID NO: 147 - DNA, 3947 bp, synthetic construct
  source 1..3947: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtaggtatggcatgagctgggtccgccaggctccagggaagaggctggagtgggtctcaaccattagtggcggaggcacctacatatactacccagactcagtgaagggccgattcaccatctccagagacaacgccaagaacaccctgtatctgcaaatgaacagcctgagagccgaggacacggctgtgtattactgtaccagagataactatggccgcaactatgattatggcatggattattggggccagggcaccctggtgaccgtgagcagcggtggtggcggttccggcggtggcggttccggtggtggcggttccgaaattgtgttgacacagtctccagccaccctgtctttgtctccaggggaaagagccaccctcacctgcagcgccaccagcagtgttagctacatccactggtatcaacagaggcctggccagagccccaggctcctcatctatagcacctccaacctggccagcggcatcccagccaggttcagtggcagtgggtctgggagcgactacactctcaccatcagcagcctagagcctgaagattttgcagtttattactgtcagcagcgtagcagctcccctttcacctttggcagcggcaccaaagtggaaattaaaacaacaacccctgcccccagacctcctaccccagcccctacaattgccagccagcctctgagcctgaggcccgaggcttgtagacctgctgctggcggagccgtgcacaccagaggactggatttcgcctgcgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataagtttaaactgccagaacatttctctggcctaactggccggtaccggaggaaaaactgtttcatacagaaggcgtggaggaaaaactgtttcatacagaaggcgtggaggaaaaactgtttcatacagaaggcgtactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtctggaagcggtgagggaagaggtagcctgctcacgtgcggtgatgtggaggagaaccctgggcctatgctctgtttgggctggatatttctttggcttgttgcaggagagcgaattaaaggatttaatatttcaggttgttccacaaaaaaactcctttggacatattctacaaggagtgaagaggaatttgtcttattttgtgatttaccagagccacagaaatcacatttctgccacagaaatcgactctcaccaaaacaagtccctgagcacctgcccttcatgggtagtaacgacctatctgatgtccaatggtaccaacaaccttcgaatggagatccattagaggacattaggaaaagctatcctcacatcattcaggacaaatgtacccttcactttttgaccccaggggtgaataattctgggtcatatatttgtagacccaagatgattaagagcccctatgatgtagcctgttgtgtcaagatgattttagaagttaagccccagacaaatgcatcctgtgagtattccgcatcacataagcaagacctacttcttgggagcactg

SEQ ID NO: 148 - DNA, 3947 bp, synthetic construct
  source 1..3947: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtaggtatggcatgagctgggtccgccaggctccagggaagaggctggagtgggtctcaaccattagtggcggaggcacctacatatactacccagactcagtgaagggccgattcaccatctccagagacaacgccaagaacaccctgtatctgcaaatgaacagcctgagagccgaggacacggctgtgtattactgtaccagagataactatggccgcaactatgattatggcatggattattggggccagggcaccctggtgaccgtgagcagcggtggtggcggttccggcggtggcggttccggtggtggcggttccgaaattgtgttgacacagtctccagccaccctgtctttgtctccaggggaaagagccaccctcacctgcagcgccaccagcagtgttagctacatccactggtatcaacagaggcctggccagagccccaggctcctcatctatagcacctccaacctggccagcggcatcccagccaggttcagtggcagtgggtctgggagcgactacactctcaccatcagcagcctagagcctgaagattttgcagtttattactgtcagcagcgtagcagctcccctttcacctttggcagcggcaccaaagtggaaattaaaaagcacctgtgtccttctccactgttccccggccctagcaagcctttctgggtcctggtggtggtgggcggagtgctggcctgctacagcctgctggtgaccgtggcctttatcatcttctgggtgaggagtaagaggagcaggctcctgcacagtgactacatgaacatgactcctagaagacctgggcctaccagaaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataagtttaaactgccagaacatttctctggcctaactggccggtaccggcttcattttttccatttactgcagaggcttcattttttccatttactgcagaggcttcattttttccatttactgcagaggcttcattttttccatttactgcagaggcttcattttttccatttactgcagaggcttcattttttccatttactgcagaactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtctggaagcggtgagggaagaggtagcctgctcacgtgcggtgatgtggaggagaaccctgggcctatgctctgtttgggctggatatttctttggcttgttgcaggagagcgaattaaaggatttaatatttcaggttgttccacaaaaaaactcctttggacatattctacaaggagtgaagaggaatttgtcttattttgtgatttaccagagccacagaaatcacatttctgccacagaaatcgactctcaccaaaacaagtccctgagcacctgcccttcatgggtagtaacgacctatctgatgtccaatggtaccaacaaccttcgaatggagatccattagaggacattaggaaaagctatcctcacatcattcaggacaaatgtacccttcactttttgaccccaggggtgaataattctgggtcatatatttgtagacccaagatgattaagagcccctatgatgtagcctgttgtgtcaagatgattttagaagttaagccccagacaaatgcatcctgtgagtattccgcatcacataagcaagacctacttcttgggagcactggctctatttctt

SEQ ID NO: 149 - DNA, 3947 bp, synthetic construct
  source 1..3947: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtaggtatggcatgagctgggtccgccaggctccagggaagaggctggagtgggtctcaaccattagtggcggaggcacctacatatactacccagactcagtgaagggccgattcaccatctccagagacaacgccaagaacaccctgtatctgcaaatgaacagcctgagagccgaggacacggctgtgtattactgtaccagagataactatggccgcaactatgattatggcatggattattggggccagggcaccctggtgaccgtgagcagcggtggtggcggttccggcggtggcggttccggtggtggcggttccgaaattgtgttgacacagtctccagccaccctgtctttgtctccaggggaaagagccaccctcacctgcagcgccaccagcagtgttagctacatccactggtatcaacagaggcctggccagagccccaggctcctcatctatagcacctccaacctggccagcggcatcccagccaggttcagtggcagtgggtctgggagcgactacactctcaccatcagcagcctagagcctgaagattttgcagtttattactgtcagcagcgtagcagctcccctttcacctttggcagcggcaccaaagtggaaattaaaaagcacctgtgtccttctccactgttccccggccctagcaagcctttctgggtcctggtggtggtgggcggagtgctggcctgctacagcctgctggtgaccgtggcctttatcatcttctgggtgaggagtaagaggagcaggctcctgcacagtgactacatgaacatgactcctagaagacctgggcctaccagaaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataagtttaaactgccagaacatttctctggcctaactggccggtaccggcttcattttttccatttactgcagaggcttcattttttccatttactgcagaggcttcattttttccatttactgcagaactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtctggaagcggtgagggaagaggtagcctgctcacgtgcggtgatgtggaggagaaccctgggcctatgctctgtttgggctggatatttctttggcttgttgcaggagagcgaattaaaggatttaatatttcaggttgttccacaaaaaaactcctttggacatattctacaaggagtgaagaggaatttgtcttattttgtgatttaccagagccacagaaatcacatttctgccacagaaatcgactctcaccaaaacaagtccctgagcacctgcccttcatgggtagtaacgacctatctgatgtccaatggtaccaacaaccttcgaatggagatccattagaggacattaggaaaagctatcctcacatcattcaggacaaatgtacccttcactttttgaccccaggggtgaataattctgggtcatatatttgtagacccaagatgattaagagcccctatgatgtagcctgttgtgtcaagatgattttagaagttaagccccagacaaatgcatcctgtgagtattccgcatcacataagcaagacctacttcttgggagcactggctctatttcttgccccagtctcagctgccaaagtgatgcacaaagtccagcggtaacctggtacaagaatggaaaactcctctctgtggaaa

SEQ ID NO: 150 - DNA, 3947 bp, synthetic construct
  source 1..3947: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtaggtatggcatgagctgggtccgccaggctccagggaagaggctggagtgggtctcaaccattagtggcggaggcacctacatatactacccagactcagtgaagggccgattcaccatctccagagacaacgccaagaacaccctgtatctgcaaatgaacagcctgagagccgaggacacggctgtgtattactgtaccagagataactatggccgcaactatgattatggcatggattattggggccagggcaccctggtgaccgtgagcagcggtggtggcggttccggcggtggcggttccggtggtggcggttccgaaattgtgttgacacagtctccagccaccctgtctttgtctccaggggaaagagccaccctcacctgcagcgccaccagcagtgttagctacatccactggtatcaacagaggcctggccagagccccaggctcctcatctatagcacctccaacctggccagcggcatcccagccaggttcagtggcagtgggtctgggagcgactacactctcaccatcagcagcctagagcctgaagattttgcagtttattactgtcagcagcgtagcagctcccctttcacctttggcagcggcaccaaagtggaaattaaaaagcacctgtgtccttctccactgttccccggccctagcaagcctttctgggtcctggtggtggtgggcggagtgctggcctgctacagcctgctggtgaccgtggcctttatcatcttctgggtgaggagtaagaggagcaggctcctgcacagtgactacatgaacatgactcctagaagacctgggcctaccagaaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataagtttaaactgccagaacatttctctggcctaactggccggtaccggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtctggaagcggtgagggaagaggtagcctgctcacgtgcggtgatgtggaggagaaccctgggcctatgctctgtttgggctggatatttctttggcttgttgcaggagagcgaattaaaggatttaatatttcaggttgttccacaaaaaaactcctttggacatattctacaaggagtgaagaggaatttgtcttattttgtgatttaccagagccacagaaatcacatttctgccacagaaatcgactctcaccaaaacaagtccctgagcacctgcccttcatgggtagtaacgacctatctgatgtccaatggtaccaacaaccttcgaatggagatccattagaggacattaggaaaagctatcctcacatcattcaggacaaatgtacccttcactttttgaccccaggggtgaataattctgggtcatatatttgtagacccaagatgattaagagcccctatgatgtagcctgttgtgtcaagatgattttagaagttaagccccagacaaatgcatcctgtgagtattccgcatcacataagcaagacctacttcttggga

SEQ ID NO: 151 - DNA, 3947 bp, synthetic construct
  source 1..3947: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtaggtatggcatgagctgggtccgccaggctccagggaagaggctggagtgggtctcaaccattagtggcggaggcacctacatatactacccagactcagtgaagggccgattcaccatctccagagacaacgccaagaacaccctgtatctgcaaatgaacagcctgagagccgaggacacggctgtgtattactgtaccagagataactatggccgcaactatgattatggcatggattattggggccagggcaccctggtgaccgtgagcagcggtggtggcggttccggcggtggcggttccggtggtggcggttccgaaattgtgttgacacagtctccagccaccctgtctttgtctccaggggaaagagccaccctcacctgcagcgccaccagcagtgttagctacatccactggtatcaacagaggcctggccagagccccaggctcctcatctatagcacctccaacctggccagcggcatcccagccaggttcagtggcagtgggtctgggagcgactacactctcaccatcagcagcctagagcctgaagattttgcagtttattactgtcagcagcgtagcagctcccctttcacctttggcagcggcaccaaagtggaaattaaaaagcacctgtgtccttctccactgttccccggccctagcaagcctttctgggtcctggtggtggtgggcggagtgctggcctgctacagcctgctggtgaccgtggcctttatcatcttctgggtgaggagtaagaggagcaggctcctgcacagtgactacatgaacatgactcctagaagacctgggcctaccagaaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataagtttaaactgccagaacatttctctggcctaactggccggtaccggaggaaaaactgtttcatacagaaggcgtggaggaaaaactgtttcatacagaaggcgtggaggaaaaactgtttcatacagaaggcgtactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtctggaagcggtgagggaagaggtagcctgctcacgtgcggtgatgtggaggagaaccctgggcctatgctctgtttgggctggatatttctttggcttgttgcaggagagcgaattaaaggatttaatatttcaggttgttccacaaaaaaactcctttggacatattctacaaggagtgaagaggaatttgtcttattttgtgatttaccagagccacagaaatcacatttctgccacagaaatcgactctcaccaaaacaagtccctgagcacctgcccttcatgggtagtaacgacctatctgatgtccaatggtaccaacaaccttcgaatggagatccattagaggacattaggaaaagctatcctcacatcattcaggacaaatgtacccttcactttttgaccccaggggtgaataattctgggtcatatatttgtagacccaagatgattaagagcccctatgatgtagcctgttgtgtcaagatgattttagaagttaagccccagacaaatgcatcctgtgagtattccgcatcacataagcaagacctacttcttgggagcactggctctatttcttgccccagtctcagctgccaaagtgatgcacaaagtccagcggtaacctggtacaagaatggaaaactcctct

SEQ ID NO: 152 - DNA, 3947 bp, synthetic construct
  source 1..3947: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtaggtatggcatgagctgggtccgccaggctccagggaagaggctggagtgggtctcaaccattagtggcggaggcacctacatatactacccagactcagtgaagggccgattcaccatctccagagacaacgccaagaacaccctgtatctgcaaatgaacagcctgagagccgaggacacggctgtgtattactgtaccagagataactatggccgcaactatgattatggcatggattattggggccagggcaccctggtgaccgtgagcagcggtggtggcggttccggcggtggcggttccggtggtggcggttccgaaattgtgttgacacagtctccagccaccctgtctttgtctccaggggaaagagccaccctcacctgcagcgccaccagcagtgttagctacatccactggtatcaacagaggcctggccagagccccaggctcctcatctatagcacctccaacctggccagcggcatcccagccaggttcagtggcagtgggtctgggagcgactacactctcaccatcagcagcctagagcctgaagattttgcagtttattactgtcagcagcgtagcagctcccctttcacctttggcagcggcaccaaagtggaaattaaaaagcacctgtgtccttctccactgttccccggccctagcaagcctttctgggtcctggtggtggtgggcggagtgctggcctgctacagcctgctggtgaccgtggcctttatcatcttctgggtgaggagtaagaggagcaggctcctgcacagtgactacatgaacatgactcctagaagacctgggcctaccagaaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgctgataagtttaaactgccagaacattactctggcctaactggccggtaccggcttcattttttccatttactgcagaggcttcattttttccatttactgcagaggcttcattttttccatttactgcagaggcttcattttttccatttactgcagaggcttcattttttccatttactgcagaggcttcattttttccatttactgcagaactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtctggaagcggtgagggaagaggtagcctgctcacgtgcggtgatgtggaggagaaccctgggcctatgctctgtttgggctggatatttctttggcttgttgcaggagagcgaattaaaggatttaatatttcaggttgttccacaaaaaaactcctttggacatattctacaaggagtgaagaggaatttgtcttattttgtgatttaccagagccacagaaatcacatttctgccacagaaatcgactctcaccaaaacaagtccctgagcacctgcccttcatgggtagtaacgacctatctgatgtccaatggtaccaacaaccttcgaatggagatccattagaggacattaggaaaagctatcctcacatcattcaggacaaatgtacccttcactttttgaccccaggggtgaataattctgggtcatatatttgtagacccaagatgattaagagcccctatgatgtagcctgttgtgtcaagatgattttagaagttaagccccagacaaatgcatcctgtgagtattccgcatcacataagcaagacctacttcttgggagcactggctctatttctt

SEQ ID NO: 153 - DNA, 3947 bp, synthetic construct
  source 1..3947: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtaggtatggcatgagctgggtccgccaggctccagggaagaggctggagtgggtctcaaccattagtggcggaggcacctacatatactacccagactcagtgaagggccgattcaccatctccagagacaacgccaagaacaccctgtatctgcaaatgaacagcctgagagccgaggacacggctgtgtattactgtaccagagataactatggccgcaactatgattatggcatggattattggggccagggcaccctggtgaccgtgagcagcggtggtggcggttccggcggtggcggttccggtggtggcggttccgaaattgtgttgacacagtctccagccaccctgtctttgtctccaggggaaagagccaccctcacctgcagcgccaccagcagtgttagctacatccactggtatcaacagaggcctggccagagccccaggctcctcatctatagcacctccaacctggccagcggcatcccagccaggttcagtggcagtgggtctgggagcgactacactctcaccatcagcagcctagagcctgaagattttgcagtttattactgtcagcagcgtagcagctcccctttcacctttggcagcggcaccaaagtggaaattaaaaagcacctgtgtccttctccactgttccccggccctagcaagcctttctgggtcctggtggtggtgggcggagtgctggcctgctacagcctgctggtgaccgtggcctttatcatcttctgggtgaggagtaagaggagcaggctcctgcacagtgactacatgaacatgactcctagaagacctgggcctaccagaaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgctgataagtttaaactgccagaacattactctggcctaactggccggtaccggcttcattttttccatttactgcagaggcttcattttttccatttactgcagaggcttcattttttccatttactgcagaactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtctggaagcggtgagggaagaggtagcctgctcacgtgcggtgatgtggaggagaaccctgggcctatgctctgtttgggctggatatttctttggcttgttgcaggagagcgaattaaaggatttaatatttcaggttgttccacaaaaaaactcctttggacatattctacaaggagtgaagaggaatttgtcttattttgtgatttaccagagccacagaaatcacatttctgccacagaaatcgactctcaccaaaacaagtccctgagcacctgcccttcatgggtagtaacgacctatctgatgtccaatggtaccaacaaccttcgaatggagatccattagaggacattaggaaaagctatcctcacatcattcaggacaaatgtacccttcactttttgaccccaggggtgaataattctgggtcatatatttgtagacccaagatgattaagagcccctatgatgtagcctgttgtgtcaagatgattttagaagttaagccccagacaaatgcatcctgtgagtattccgcatcacataagcaagacctacttcttgggagcactggctctatttcttgccccagtctcagctgccaaagtgatgcacaaagtccagcggtaacctggtacaagaatggaaaactcctctctgtggaaa

SEQ ID NO: 154 - DNA, 3947 bp, synthetic construct
  source 1..3947: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtaggtatggcatgagctgggtccgccaggctccagggaagaggctggagtgggtctcaaccattagtggcggaggcacctacatatactacccagactcagtgaagggccgattcaccatctccagagacaacgccaagaacaccctgtatctgcaaatgaacagcctgagagccgaggacacggctgtgtattactgtaccagagataactatggccgcaactatgattatggcatggattattggggccagggcaccctggtgaccgtgagcagcggtggtggcggttccggcggtggcggttccggtggtggcggttccgaaattgtgttgacacagtctccagccaccctgtctttgtctccaggggaaagagccaccctcacctgcagcgccaccagcagtgttagctacatccactggtatcaacagaggcctggccagagccccaggctcctcatctatagcacctccaacctggccagcggcatcccagccaggttcagtggcagtgggtctgggagcgactacactctcaccatcagcagcctagagcctgaagattttgcagtttattactgtcagcagcgtagcagctcccctttcacctttggcagcggcaccaaagtggaaattaaaaagcacctgtgtccttctccactgttccccggccctagcaagcctttctgggtcctggtggtggtgggcggagtgctggcctgctacagcctgctggtgaccgtggcctttatcatcttctgggtgaggagtaagaggagcaggctcctgcacagtgactacatgaacatgactcctagaagacctgggcctaccagaaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgctgataagtttaaactgccagaacattactctggcctaactggccggtaccggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtctggaagcggtgagggaagaggtagcctgctcacgtgcggtgatgtggaggagaaccctgggcctatgctctgtttgggctggatatttctttggcttgttgcaggagagcgaattaaaggatttaatatttcaggttgttccacaaaaaaactcctttggacatattctacaaggagtgaagaggaatttgtcttattttgtgatttaccagagccacagaaatcacatttctgccacagaaatcgactctcaccaaaacaagtccctgagcacctgcccttcatgggtagtaacgacctatctgatgtccaatggtaccaacaaccttcgaatggagatccattagaggacattaggaaaagctatcctcacatcattcaggacaaatgtacccttcactttttgaccccaggggtgaataattctgggtcatatatttgtagacccaagatgattaagagcccctatgatgtagcctgttgtgtcaagatgattttagaagttaagccccagacaaatgcatcctgtgagtattccgcatcacataagcaagacctacttcttggga

SEQ ID NO: 155 - DNA, 3947 bp, synthetic construct
  source 1..3947: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtaggtatggcatgagctgggtccgccaggctccagggaagaggctggagtgggtctcaaccattagtggcggaggcacctacatatactacccagactcagtgaagggccgattcaccatctccagagacaacgccaagaacaccctgtatctgcaaatgaacagcctgagagccgaggacacggctgtgtattactgtaccagagataactatggccgcaactatgattatggcatggattattggggccagggcaccctggtgaccgtgagcagcggtggtggcggttccggcggtggcggttccggtggtggcggttccgaaattgtgttgacacagtctccagccaccctgtctttgtctccaggggaaagagccaccctcacctgcagcgccaccagcagtgttagctacatccactggtatcaacagaggcctggccagagccccaggctcctcatctatagcacctccaacctggccagcggcatcccagccaggttcagtggcagtgggtctgggagcgactacactctcaccatcagcagcctagagcctgaagattttgcagtttattactgtcagcagcgtagcagctcccctttcacctttggcagcggcaccaaagtggaaattaaaaagcacctgtgtccttctccactgttccccggccctagcaagcctttctgggtcctggtggtggtgggcggagtgctggcctgctacagcctgctggtgaccgtggcctttatcatcttctgggtgaggagtaagaggagcaggctcctgcacagtgactacatgaacatgactcctagaagacctgggcctaccagaaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgctgataagtttaaactgccagaacattactctggcctaactggccggtaccggaggaaaaactgtttcatacagaaggcgtggaggaaaaactgtttcatacagaaggcgtggaggaaaaactgtttcatacagaaggcgtactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtctggaagcggtgagggaagaggtagcctgctcacgtgcggtgatgtggaggagaaccctgggcctatgctctgtttgggctggatatttctttggcttgttgcaggagagcgaattaaaggatttaatatttcaggttgttccacaaaaaaactcctttggacatattctacaaggagtgaagaggaatttgtcttattttgtgatttaccagagccacagaaatcacatttctgccacagaaatcgactctcaccaaaacaagtccctgagcacctgcccttcatgggtagtaacgacctatctgatgtccaatggtaccaacaaccttcgaatggagatccattagaggacattaggaaaagctatcctcacatcattcaggacaaatgtacccttcactttttgaccccaggggtgaataattctgggtcatatatttgtagacccaagatgattaagagcccctatgatgtagcctgttgtgtcaagatgattttagaagttaagccccagacaaatgcatcctgtgagtattccgcatcacataagcaagacctacttcttgggagcactggctctatttcttgccccagtctcagctgccaaagtgatgcacaaagtccagcggtaacctggtacaagaatggaaaactcctct

SEQ ID NO: 156 - DNA, 3947 bp, synthetic construct
  source 1..3947: mol_type = other DNA
atggccttaccagtgaccgccttgctcctgccgctggccttgctgctccacgccgccaggccgcaggtgcagctggttgaatctggcggcggacttgtgaagcctggcggatctctgagactgagctgtgccgccagcggcttcacctttagcacatacgccatgagctggatcagacaggcccctggcaaaggcctggaatgggtggcgtctattggcagagccggcagcacctactacagcgactctgtgaagggcagattcaccatcagccgggacaacgccaagaacagcctgtacctgcagatgaactccctgagagccgaggacaccgccgtgtactattgtgccagaggacccatctacaacgactacgacgagttcgcctattggggccagggcacactggtcacagtttctagcggcggtggcggaagcggaggcggtggctccggtggcggaggcagcgaaattgtgctgacacagagccccgccacactgtcactttctccaggcgaaagagccacactgagctgcaagagcagccagagcgtgctgtactccagcaaccagaagaactacctggcctggtatcagcagaagcccggccaagctcctcggctgctgatctattgggccagcacaagagagagcggcatccctgccagattttctggcagcggctctggcaccgatttcaccctgaccataagcagcctggaacctgaggacttcgccgtgtattactgccaccagtacctgagcagcctgacctttggcggaggcaccaaggtggaaatcaagcggacaacaacccctgcccccagacctcctaccccagcccctacaattgccagccagcctctgagcctgaggcccgaggcttgtagacctgctgctggcggagccgtgcacaccagaggactggatttcgcctgcgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataagtttaaactgccagaacatttctctggcctaactggccggtaccggcttcattttttccatttactgcagaggcttcattttttccatttactgcagaggcttcattttttccatttactgcagaggcttcattttttccatttactgcagaggcttcattttttccatttactgcagaggcttcattttttccatttactgcagaactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtctggaagcggtgagggaagaggtagcctgctcacgtgcggtgatgtggaggagaaccctgggcctatgctctgtttgggctggatatttctttggcttgttgcaggagagcgaattaaaggatttaatatttcaggttgttccacaaaaaaactcctttggacatattctacaaggagtgaagaggaatttgtcttattttgtgatttaccagagccacagaaatcacatttctgccacagaaatcgactctcaccaaaacaagtccctgagcacctgcccttcatgggtagtaacgacctatctgatgtccaatggtaccaacaaccttcgaatggagatccattagaggacattaggaaaagctatcctcacatcattcaggacaaatgtacccttcactttttgaccccaggggtgaataattctgggtcatatatttgtagacccaagatgattaagagcccctatgatgtagcctgttgtgtca

SEQ ID NO: 157 - DNA, 3947 bp, synthetic construct
  source 1..3947: mol_type = other DNA
atggccttaccagtgaccgccttgctcctgccgctggccttgctgctccacgccgccaggccgcaggtgcagctggttgaatctggcggcggacttgtgaagcctggcggatctctgagactgagctgtgccgccagcggcttcacctttagcacatacgccatgagctggatcagacaggcccctggcaaaggcctggaatgggtggcgtctattggcagagccggcagcacctactacagcgactctgtgaagggcagattcaccatcagccgggacaacgccaagaacagcctgtacctgcagatgaactccctgagagccgaggacaccgccgtgtactattgtgccagaggacccatctacaacgactacgacgagttcgcctattggggccagggcacactggtcacagtttctagcggcggtggcggaagcggaggcggtggctccggtggcggaggcagcgaaattgtgctgacacagagccccgccacactgtcactttctccaggcgaaagagccacactgagctgcaagagcagccagagcgtgctgtactccagcaaccagaagaactacctggcctggtatcagcagaagcccggccaagctcctcggctgctgatctattgggccagcacaagagagagcggcatccctgccagattttctggcagcggctctggcaccgatttcaccctgaccataagcagcctggaacctgaggacttcgccgtgtattactgccaccagtacctgagcagcctgacctttggcggaggcaccaaggtggaaatcaagcggacaacaacccctgcccccagacctcctaccccagcccctacaattgccagccagcctctgagcctgaggcccgaggcttgtagacctgctgctggcggagccgtgcacaccagaggactggatttcgcctgcgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataagtttaaactgccagaacatttctctggcctaactggccggtaccggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtctggaagcggtgagggaagaggtagcctgctcacgtgcggtgatgtggaggagaaccctgggcctatgctctgtttgggctggatatttctttggcttgttgcaggagagcgaattaaaggatttaatatttcaggttgttccacaaaaaaactcctttggacatattctacaaggagtgaagaggaatttgtcttattttgtgatttaccagagccacagaaatcacatttctgccacagaaatcgactctcaccaaaacaagtccctgagcacctgcccttcatgggtagtaacgacctatctgatgtccaatggtaccaacaaccttcgaatggagatccattagaggacattaggaaaagctatcctcacatcattcaggacaaatgtacccttcactttttgaccccaggggtgaataattctgggtcatatatttgtagacccaagatgattaagagcccctatg

SEQ ID NO: 158 - DNA, 3947 bp, synthetic construct; identical to SEQ ID NO: 157
  source 1..3947: mol_type = other DNA

SEQ ID NO: 159 - DNA, 3947 bp, synthetic construct
  source 1..3947: mol_type = other DNA
atggccttaccagtgaccgccttgctcctgccgctggccttgctgctccacgccgccaggccgcaggtgcagctggttgaatctggcggcggacttgtgaagcctggcggatctctgagactgagctgtgccgccagcggcttcacctttagcacatacgccatgagctggatcagacaggcccctggcaaaggcctggaatgggtggcgtctattggcagagccggcagcacctactacagcgactctgtgaagggcagattcaccatcagccgggacaacgccaagaacagcctgtacctgcagatgaactccctgagagccgaggacaccgccgtgtactattgtgccagaggacccatctacaacgactacgacgagttcgcctattggggccagggcacactggtcacagtttctagcggcggtggcggaagcggaggcggtggctccggtggcggaggcagcgaaattgtgctgacacagagccccgccacactgtcactttctccaggcgaaagagccacactgagctgcaagagcagccagagcgtgctgtactccagcaaccagaagaactacctggcctggtatcagcagaagcccggccaagctcctcggctgctgatctattgggccagcacaagagagagcggcatccctgccagattttctggcagcggctctggcaccgatttcaccctgaccataagcagcctggaacctgaggacttcgccgtgtattactgccaccagtacctgagcagcctgacctttggcggaggcaccaaggtggaaatcaagcggacaacaacccctgcccccagacctcctaccccagcccctacaattgccagccagcctctgagcctgaggcccgaggcttgtagacctgctgctggcggagccgtgcacaccagaggactggatttcgcctgcgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataagtttaaactgccagaacatttctctggcctaactggccggtaccggaggaaaaactgtttcatacagaaggcgtggaggaaaaactgtttcatacagaaggcgtggaggaaaaactgtttcatacagaaggcgtactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtctggaagcggtgagggaagaggtagcctgctcacgtgcggtgatgtggaggagaaccctgggcctatgctctgtttgggctggatatttctttggcttgttgcaggagagcgaattaaaggatttaatatttcaggttgttccacaaaaaaactcctttggacatattctacaaggagtgaagaggaatttgtcttattttgtgatttaccagagccacagaaatcacatttctgccacagaaatcgactctcaccaaaacaagtccctgagcacctgcccttcatgggtagtaacgacctatctgatgtccaatggtaccaacaaccttcgaatggagatccattagaggacattaggaaaagctatcctcacatcattcaggacaaatgtacccttcactttttgaccccaggggtgaataattctgggtcatatatttgtagacccaagatgattaagagcccctatgatgtagcctgttgtgtcaagatgattttagaagttaagccccagacaaatgcatcctgtgagtattccgcatcacataagcaagacctac

SEQ ID NO: 160 - DNA, 3947 bp, synthetic construct
  source 1..3947: mol_type = other DNA
atggccttaccagtgaccgccttgctcctgccgctggccttgctgctccacgccgccaggccgcaggtgcagctggttgaatctggcggcggacttgtgaagcctggcggatctctgagactgagctgtgccgccagcggcttcacctttagcacatacgccatgagctggatcagacaggcccctggcaaaggcctggaatgggtggcgtctattggcagagccggcagcacctactacagcgactctgtgaagggcagattcaccatcagccgggacaacgccaagaacagcctgtacctgcagatgaactccctgagagccgaggacaccgccgtgtactattgtgccagaggacccatctacaacgactacgacgagttcgcctattggggccagggcacactggtcacagtttctagcggcggtggcggaagcggaggcggtggctccggtggcggaggcagcgaaattgtgctgacacagagccccgccacactgtcactttctccaggcgaaagagccacactgagctgcaagagcagccagagcgtgctgtactccagcaaccagaagaactacctggcctggtatcagcagaagcccggccaagctcctcggctgctgatctattgggccagcacaagagagagcggcatccctgccagattttctggcagcggctctggcaccgatttcaccctgaccataagcagcctggaacctgaggacttcgccgtgtattactgccaccagtacctgagcagcctgacctttggcggaggcaccaaggtggaaatcaagcggaagcacctgtgtccttctccactgttccccggccctagcaagcctttctgggtcctggtggtggtgggcggagtgctggcctgctacagcctgctggtgaccgtggcctttatcatcttctgggtgaggagtaagaggagcaggctcctgcacagtgactacatgaacatgactcctagaagacctgggcctaccagaaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataagtttaaactgccagaacatttctctggcctaactggccggtaccggcttcattttttccatttactgcagaggcttcattttttccatttactgcagaggcttcattttttccatttactgcagaggcttcattttttccatttactgcagaggcttcattttttccatttactgcagaggcttcattttttccatttactgcagaactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtctggaagcggtgagggaagaggtagcctgctcacgtgcggtgatgtggaggagaaccctgggcctatgctctgtttgggctggatatttctttggcttgttgcaggagagcgaattaaaggatttaatatttcaggttgttccacaaaaaaactcctttggacatattctacaaggagtgaagaggaatttgtcttattttgtgatttaccagagccacagaaatcacatttctgccacagaaatcgactctcaccaaaacaagtccctgagcacctgcccttcatgggtagtaacgacctatctgatgtccaatggtaccaacaaccttcgaatggagatccattagaggacattaggaaaagctatcctcacatcattcaggacaaatgtacccttcactttttgaccccaggggtgaataattctgggtcatatatttgtagacccaagatgattaagagcccctatgatgtagcctgttgtgtcaagatgattttagaagttaagccccagacaaatgcatcctgtgagtattccgcatcacataagcaagacctacttcttgggagca

SEQ ID NO: 161 - DNA, 3947 bp, synthetic construct
  source 1..3947: mol_type = other DNA
atggccttaccagtgaccgccttgctcctgccgctggccttgctgctccacgccgccaggccgcaggtgcagctggttgaatctggcggcggacttgtgaagcctggcggatctctgagactgagctgtgccgccagcggcttcacctttagcacatacgccatgagctggatcagacaggcccctggcaaaggcctggaatgggtggcgtctattggcagagccggcagcacctactacagcgactctgtgaagggcagattcaccatcagccgggacaacgccaagaacagcctgtacctgcagatgaactccctgagagccgaggacaccgccgtgtactattgtgccagaggacccatctacaacgactacgacgagttcgcctattggggccagggcacactggtcacagtttctagcggcggtggcggaagcggaggcggtggctccggtggcggaggcagcgaaattgtgctgacacagagccccgccacactgtcactttctccaggcgaaagagccacactgagctgcaagagcagccagagcgtgctgtactccagcaaccagaagaactacctggcctggtatcagcagaagcccggccaagctcctcggctgctgatctattgggccagcacaagagagagcggcatccctgccagattttctggcagcggctctggcaccgatttcaccctgaccataagcagcctggaacctgaggacttcgccgtgtattactgccaccagtacctgagcagcctgacctttggcggaggcaccaaggtggaaatcaagcggaagcacctgtgtccttctccactgttccccggccctagcaagcctttctgggtcctggtggtggtgggcggagtgctggcctgctacagcctgctggtgaccgtggcctttatcatcttctgggtgaggagtaagaggagcaggctcctgcacagtgactacatgaacatgactcctagaagacctgggcctaccagaaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataagtttaaactgccagaacatttctctggcctaactggccggtaccggcttcattttttccatttactgcagaggcttcattttttccatttactgcagaggcttcattttttccatttactgcagaactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtctggaagcggtgagggaagaggtagcctgctcacgtgcggtgatgtggaggagaaccctgggcctatgctctgtttgggctggatatttctttggcttgttgcaggagagcgaattaaaggatttaatatttcaggttgttccacaaaaaaactcctttggacatattctacaaggagtgaagaggaatttgtcttattttgtgatttaccagagccacagaaatcacatttctgccacagaaatcgactctcaccaaaacaagtccctgagcacctgcccttcatgggtagtaacgacctatctgatgtccaatggtaccaacaaccttcgaatggagatccattagaggacattaggaaaagctatcctcacatcattcaggacaaatgtacccttcactttttgaccccaggggtgaataattctgggtcatatatttgtagacccaagatgattaagagcccctatgatgtagcctgttgtgtcaagatgattttagaagttaagccccagacaaatgcatcctgtgagtattccgcatcacataagcaagacctacttcttgggagcactggctctatttcttgccccagtctcagctgccaaagtgatgcacaaagtccagcggtaacctggtacaagaatggaaaac

SEQ ID NO: 162 - DNA, 3947 bp, synthetic construct
  source 1..3947: mol_type = other DNA
atggccttaccagtgaccgccttgctcctgccgctggccttgctgctccacgccgccaggccgcaggtgcagctggttgaatctggcggcggacttgtgaagcctggcggatctctgagactgagctgtgccgccagcggcttcacctttagcacatacgccatgagctggatcagacaggcccctggcaaaggcctggaatgggtggcgtctattggcagagccggcagcacctactacagcgactctgtgaagggcagattcaccatcagccgggacaacgccaagaacagcctgtacctgcagatgaactccctgagagccgaggacaccgccgtgtactattgtgccagaggacccatctacaacgactacgacgagttcgcctattggggccagggcacactggtcacagtttctagcggcggtggcggaagcggaggcggtggctccggtggcggaggcagcgaaattgtgctgacacagagccccgccacactgtcactttctccaggcgaaagagccacactgagctgcaagagcagccagagcgtgctgtactccagcaaccagaagaactacctggcctggtatcagcagaagcccggccaagctcctcggctgctgatctattgggccagcacaagagagagcggcatccctgccagattttctggcagcggctctggcaccgatttcaccctgaccataagcagcctggaacctgaggacttcgccgtgtattactgccaccagtacctgagcagcctgacctttggcggaggcaccaaggtggaaatcaagcggaagcacctgtgtccttctccactgttccccggccctagcaagcctttctgggtcctggtggtggtgggcggagtgctggcctgctacagcctgctggtgaccgtggcctttatcatcttctgggtgaggagtaagaggagcaggctcctgcacagtgactacatgaacatgactcctagaagacctgggcctaccagaaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataagtttaaactgccagaacatttctctggcctaactggccggtaccggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtctggaagcggtgagggaagaggtagcctgctcacgtgcggtgatgtggaggagaaccctgggcctatgctctgtttgggctggatatttctttggcttgttgcaggagagcgaattaaaggatttaatatttcaggttgttccacaaaaaaactcctttggacatattctacaaggagtgaagaggaatttgtcttattttgtgatttaccagagccacagaaatcacatttctgccacagaaatcgactctcaccaaaacaagtccctgagcacctgcccttcatgggtagtaacgacctatctgatgtccaatggtaccaacaaccttcgaatggagatccattagaggacattaggaaaagctatcctcacatcattcaggacaaatgtacccttcactttttgaccccaggggtgaataattctgggtcatatatttgtagacccaagatgattaagagcccctatgatgtagcctgttgtgtcaagatgattttagaagttaagccccagacaaatgcatcctgtgagtattccgcatcacataagcaag

SEQ ID NO: 163 - DNA, 3947 bp, synthetic construct
  source 1..3947: mol_type = other DNA
atggccttaccagtgaccgccttgctcctgccgctggccttgctgctccacgccgccaggccgcaggtgcagctggttgaatctggcggcggacttgtgaagcctggcggatctctgagactgagctgtgccgccagcggcttcacctttagcacatacgccatgagctggatcagacaggcccctggcaaaggcctggaatgggtggcgtctattggcagagccggcagcacctactacagcgactctgtgaagggcagattcaccatcagccgggacaacgccaagaacagcctgtacctgcagatgaactccctgagagccgaggacaccgccgtgtactattgtgccagaggacccatctacaacgactacgacgagttcgcctattggggccagggcacactggtcacagtttctagcggcggtggcggaagcggaggcggtggctccggtggcggaggcagcgaaattgtgctgacacagagccccgccacactgtcactttctccaggcgaaagagccacactgagctgcaagagcagccagagcgtgctgtactccagcaaccagaagaactacctggcctggtatcagcagaagcccggccaagctcctcggctgctgatctattgggccagcacaagagagagcggcatccctgccagattttctggcagcggctctggcaccgatttcaccctgaccataagcagcctggaacctgaggacttcgccgtgtattactgccaccagtacctgagcagcctgacctttggcggaggcaccaaggtggaaatcaagcggaagcacctgtgtccttctccactgttccccggccctagcaagcctttctgggtcctggtggtggtgggcggagtgctggcctgctacagcctgctggtgaccgtggcctttatcatcttctgggtgaggagtaagaggagcaggctcctgcacagtgactacatgaacatgactcctagaagacctgggcctaccagaaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataagtttaaactgccagaacatttctctggcctaactggccggtaccggaggaaaaactgtttcatacagaaggcgtggaggaaaaactgtttcatacagaaggcgtggaggaaaaactgtttcatacagaaggcgtactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtctggaagcggtgagggaagaggtagcctgctcacgtgcggtgatgtggaggagaaccctgggcctatgctctgtttgggctggatatttctttggcttgttgcaggagagcgaattaaaggatttaatatttcaggttgttccacaaaaaaactcctttggacatattctacaaggagtgaagaggaatttgtcttattttgtgatttaccagagccacagaaatcacatttctgccacagaaatcgactctcaccaaaacaagtccctgagcacctgcccttcatgggtagtaacgacctatctgatgtccaatggtaccaacaaccttcgaatggagatccattagaggacattaggaaaagctatcctcacatcattcaggacaaatgtacccttcactttttgaccccaggggtgaataattctgggtcatatatttgtagacccaagatgattaagagcccctatgatgtagcctgttgtgtcaagatgattttagaagttaagccccagacaaatgcatcctgtgagtattccgcatcacataagcaagacctacttcttgggagcactggctctatttcttgccccagtctcagctgccaaagtgatgcacaaagtccagcggtaacctggtacaaga

SEQ ID NO: 164 - DNA, 3947 bp, synthetic construct
  source 1..3947: mol_type = other DNA
atggccttaccagtgaccgccttgctcctgccgctggccttgctgctccacgccgccaggccgcaggtgcagctggttgaatctggcggcggacttgtgaagcctggcggatctctgagactgagctgtgccgccagcggcttcacctttagcacatacgccatgagctggatcagacaggcccctggcaaaggcctggaatgggtggcgtctattggcagagccggcagcacctactacagcgactctgtgaagggcagattcaccatcagccgggacaacgccaagaacagcctgtacctgcagatgaactccctgagagccgaggacaccgccgtgtactattgtgccagaggacccatctacaacgactacgacgagttcgcctattggggccagggcacactggtcacagtttctagcggcggtggcggaagcggaggcggtggctccggtggcggaggcagcgaaattgtgctgacacagagccccgccacactgtcactttctccaggcgaaagagccacactgagctgcaagagcagccagagcgtgctgtactccagcaaccagaagaactacctggcctggtatcagcagaagcccggccaagctcctcggctgctgatctattgggccagcacaagagagagcggcatccctgccagattttctggcagcggctctggcaccgatttcaccctgaccataagcagcctggaacctgaggacttcgccgtgtattactgccaccagtacctgagcagcctgacctttggcggaggcaccaaggtggaaatcaagcggaagcacctgtgtccttctccactgttccccggccctagcaagcctttctgggtcctggtggtggtgggcggagtgctggcctgctacagcctgctggtgaccgtggcctttatcatcttctgggtgaggagtaagaggagcaggctcctgcacagtgactacatgaacatgactcctagaagacctgggcctaccagaaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgctgataagtttaaactgccagaacattactctggcctaactggccggtaccggcttcattttttccatttactgcagaggcttcattttttccatttactgcagaggcttcattttttccatttactgcagaggcttcattttttccatttactgcagaggcttcattttttccatttactgcagaggcttcattttttccatttactgcagaactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtctggaagcggtgagggaagaggtagcctgctcacgtgcggtgatgtggaggagaaccctgggcctatgctctgtttgggctggatatttctttggcttgttgcaggagagcgaattaaaggatttaatatttcaggttgttccacaaaaaaactcctttggacatattctacaaggagtgaagaggaatttgtcttattttgtgatttaccagagccacagaaatcacatttctgccacagaaatcgactctcaccaaaacaagtccctgagcacctgcccttcatgggtagtaacgacctatctgatgtccaatggtaccaacaaccttcgaatggagatccattagaggacattaggaaaagctatcctcacatcattcaggacaaatgtacccttcactttttgaccccaggggtgaataattctgggtcatatatttgtagacccaagatgattaagagcccctatgatgtagcctgttgtgtcaagatgattttagaagttaagccccagacaaatgcatcctgtgagtattccgcatcacataagcaagacctacttcttgggagca

SEQ ID NO: 165 - DNA, 3947 bp, synthetic construct
  source 1..3947: mol_type = other DNA
atggccttaccagtgaccgccttgctcctgccgctggccttgctgctccacgccgccaggccgcaggtgcagctggttgaatctggcggcggacttgtgaagcctggcggatctctgagactgagctgtgccgccagcggcttcacctttagcacatacgccatgagctggatcagacaggcccctggcaaaggcctggaatgggtggcgtctattggcagagccggcagcacctactacagcgactctgtgaagggcagattcaccatcagccgggacaacgccaagaacagcctgtacctgcagatgaactccctgagagccgaggacaccgccgtgtactattgtgccagaggacccatctacaacgactacgacgagttcgcctattggggccagggcacactggtcacagtttctagcggcggtggcggaagcggaggcggtggctccggtggcggaggcagcgaaattgtgctgacacagagccccgccacactgtcactttctccaggcgaaagagccacactgagctgcaagagcagccagagcgtgctgtactccagcaaccagaagaactacctggcctggtatcagcagaagcccggccaagctcctcggctgctgatctattgggccagcacaagagagagcggcatccctgccagattttctggcagcggctctggcaccgatttcaccctgaccataagcagcctggaacctgaggacttcgccgtgtattactgccaccagtacctgagcagcctgacctttggcggaggcaccaaggtggaaatcaagcggaagcacctgtgtccttctccactgttccccggccctagcaagcctttctgggtcctggtggtggtgggcggagtgctggcctgctacagcctgctggtgaccgtggcctttatcatcttctgggtgaggagtaagaggagcaggctcctgcacagtgactacatgaacatgactcctagaagacctgggcctaccagaaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgctgataagtttaaactgccagaacattactctggcctaactggccggtaccggcttcattttttccatttactgcagaggcttcattttttccatttactgcagaggcttcattttttccatttactgcagaactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtctggaagcggtgagggaagaggtagcctgctcacgtgcggtgatgtggaggagaaccctgggcctatgctctgtttgggctggatatttctttggcttgttgcaggagagcgaattaaaggatttaatatttcaggttgttccacaaaaaaactcctttggacatattctacaaggagtgaagaggaatttgtcttattttgtgatttaccagagccacagaaatcacatttctgccacagaaatcgactctcaccaaaacaagtccctgagcacctgcccttcatgggtagtaacgacctatctgatgtccaatggtaccaacaaccttcgaatggagatccattagaggacattaggaaaagctatcctcacatcattcaggacaaatgtacccttcactttttgaccccaggggtgaataattctgggtcatatatttgtagacccaagatgattaagagcccctatgatgtagcctgttgtgtcaagatgattttagaagttaagccccagacaaatgcatcctgtgagtattccgcatcacataagcaagacctacttcttgggagcactggctctatttcttgccccagtctcagctgccaaagtgatgcacaaagtccagcggtaacctggtacaagaatggaaaac

SEQ ID NO: 166 - DNA, 3947 bp, synthetic construct
  source 1..3947: mol_type = other DNA
atggccttaccagtgaccgccttgctcctgccgctggccttgctgctccacgccgccaggccgcaggtgcagctggttgaatctggcggcggacttgtgaagcctggcggatctctgagactgagctgtgccgccagcggcttcacctttagcacatacgccatgagctggatcagacaggcccctggcaaaggcctggaatgggtggcgtctattggcagagccggcagcacctactacagcgactctgtgaagggcagattcaccatcagccgggacaacgccaagaacagcctgtacctgcagatgaactccctgagagccgaggacaccgccgtgtactattgtgccagaggacccatctacaacgactacgacgagttcgcctattggggccagggcacactggtcacagtttctagcggcggtggcggaagcggaggcggtggctccggtggcggaggcagcgaaattgtgctgacacagagccccgccacactgtcactttctccaggcgaaagagccacactgagctgcaagagcagccagagcgtgctgtactccagcaaccagaagaactacctggcctggtatcagcagaagcccggccaagctcctcggctgctgatctattgggccagcacaagagagagcggcatccctgccagattttctggcagcggctctggcaccgatttcaccctgaccataagcagcctggaacctgaggacttcgccgtgtattactgccaccagtacctgagcagcctgacctttggcggaggcaccaaggtggaaatcaagcggaagcacctgtgtccttctccactgttccccggccctagcaagcctttctgggtcctggtggtggtgggcggagtgctggcctgctacagcctgctggtgaccgtggcctttatcatcttctgggtgaggagtaagaggagcaggctcctgcacagtgactacatgaacatgactcctagaagacctgggcctaccagaaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgctgataagtttaaactgccagaacattactctggcctaactggccggtaccggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtctggaagcggtgagggaagaggtagcctgctcacgtgcggtgatgtggaggagaaccctgggcctatgctctgtttgggctggatatttctttggcttgttgcaggagagcgaattaaaggatttaatatttcaggttgttccacaaaaaaactcctttggacatattctacaaggagtgaagaggaatttgtcttattttgtgatttaccagagccacagaaatcacatttctgccacagaaatcgactctcaccaaaacaagtccctgagcacctgcccttcatgggtagtaacgacctatctgatgtccaatggtaccaacaaccttcgaatggagatccattagaggacattaggaaaagctatcctcacatcattcaggacaaatgtacccttcactttttgaccccaggggtgaataattctgggtcatatatttgtagacccaagatgattaagagcccctatgatgtagcctgttgtgtcaagatgattttagaagttaagccccagacaaatgcatcctgtgagtattccgcatcacataagcaag

SEQ ID NO: 167 - DNA, 3947 bp, synthetic construct
  source 1..3947: mol_type = other DNA
atggccttaccagtgaccgccttgctcctgccgctggccttgctgctccacgccgccaggccgcaggtgcagctggttgaatctggcggcggacttgtgaagcctggcggatctctgagactgagctgtgccgccagcggcttcacctttagcacatacgccatgagctggatcagacaggcccctggcaaaggcctggaatgggtggcgtctattggcagagccggcagcacctactacagcgactctgtgaagggcagattcaccatcagccgggacaacgccaagaacagcctgtacctgcagatgaactccctgagagccgaggacaccgccgtgtactattgtgccagaggacccatctacaacgactacgacgagttcgcctattggggccagggcacactggtcacagtttctagcggcggtggcggaagcggaggcggtggctccggtggcggaggcagcgaaattgtgctgacacagagccccgccacactgtcactttctccaggcgaaagagccacactgagctgcaagagcagccagagcgtgctgtactccagcaaccagaagaactacctggcctggtatcagcagaagcccggccaagctcctcggctgctgatctattgggccagcacaagagagagcggcatccctgccagattttctggcagcggctctggcaccgatttcaccctgaccataagcagcctggaacctgaggacttcgccgtgtattactgccaccagtacctgagcagcctgacctttggcggaggcaccaaggtggaaatcaagcggaagcacctgtgtccttctccactgttccccggccctagcaagcctttctgggtcctggtggtggtgggcggagtgctggcctgctacagcctgctggtgaccgtggcctttatcatcttctgggtgaggagtaagaggagcaggctcctgcacagtgactacatgaacatgactcctagaagacctgggcctaccagaaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgctgataagtttaaactgccagaacattactctggcctaactggccggtaccggaggaaaaactgtttcatacagaaggcgtggaggaaaaactgtttcatacagaaggcgtggaggaaaaactgtttcatacagaaggcgtactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtctggaagcggtgagggaagaggtagcctgctcacgtgcggtgatgtggaggagaaccctgggcctatgctctgtttgggctggatatttctttggcttgttgcaggagagcgaattaaaggatttaatatttcaggttgttccacaaaaaaactcctttggacatattctacaaggagtgaagaggaatttgtcttattttgtgatttaccagagccacagaaatcacatttctgccacagaaatcgactctcaccaaaacaagtccctgagcacctgcccttcatgggtagtaacgacctatctgatgtccaatggtaccaacaaccttcgaatggagatccattagaggacattaggaaaagctatcctcacatcattcaggacaaatgtacccttcactttttgaccccaggggtgaataattctgggtcatatatttgtagacccaagatgattaagagcccctatgatgtagcctgttgtgtcaagatgattttagaagttaagccccagacaaatgcatcctgtgagtattccgcatcacataagcaagacctacttcttgggagcactggctctatttcttgccccagtctcagctgccaaagtgatgcacaaagtccagcggtaacctggtacaaga

SEQ ID NO: 168 - DNA, 3469 bp, synthetic construct
  source 1..3469: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtggctatgccatgagctgggtccgccaggctccagggaaggggctggagtgggtctcaaccattagtagtggcggaacctacatatactaccccgactcagtgaagggccgattcaccatctccagagacaacgccaagaactcactgtatctgcaaatgaacagcctgagagccgaggacacggccgtgtattactgtgcgagacttgggggggataattactacgaatacttcgatgtctggggcaaagggaccacggtcaccgtctcctccggcggtggcggatccggcggtggcggatccggcggtggcggatccgacattgtgctgacccagtctccagcctccttggccgtgtctccaggacagagggccaccatcacctgcagagccagtaagagtgtcagtaccagcggatactcctacatgcactggtatcagcagaaaccaggacaacctcctaaactcctgatttacctggcatccaatctggagagcggggtcccagccaggttcagcggcagtgggtctgggaccgatttcaccctcacaattaatcctgtggaagctaatgatactgcaaattattactgtcagcacagtagggagctgcctttcacattcggcggagggaccaaggtggagatcaaacgaactacaacaacccctgcccccagacctcctaccccagcccctacaattgccagccagcctctgagcctgaggcccgaggcttgtagacctgctgctggcggagccgtgcacaccagaggactggatttcgcctgcgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgctgataagtttaaactgccagaacatttctctggcctaactggccggtaccggcttcattttttccatttactgcagaggcttcattttttccatttactgcagaggcttcattttttccatttactgcagaggcttcattttttccatttactgcagaggcttcattttttccatttactgcagaggcttcattttttccatttactgcagaactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttgataa

SEQ ID NO: 169 - DNA, 3388 bp, synthetic construct
  source 1..3388: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtggctatgccatgagctgggtccgccaggctccagggaaggggctggagtgggtctcaaccattagtagtggcggaacctacatatactaccccgactcagtgaagggccgattcaccatctccagagacaacgccaagaactcactgtatctgcaaatgaacagcctgagagccgaggacacggccgtgtattactgtgcgagacttgggggggataattactacgaatacttcgatgtctggggcaaagggaccacggtcaccgtctcctccggcggtggcggatccggcggtggcggatccggcggtggcggatccgacattgtgctgacccagtctccagcctccttggccgtgtctccaggacagagggccaccatcacctgcagagccagtaagagtgtcagtaccagcggatactcctacatgcactggtatcagcagaaaccaggacaacctcctaaactcctgatttacctggcatccaatctggagagcggggtcccagccaggttcagcggcagtgggtctgggaccgatttcaccctcacaattaatcctgtggaagctaatgatactgcaaattattactgtcagcacagtagggagctgcctttcacattcggcggagggaccaaggtggagatcaaacgaactacaacaacccctgcccccagacctcctaccccagcccctacaattgccagccagcctctgagcctgaggcccgaggcttgtagacctgctgctggcggagccgtgcacaccagaggactggatttcgcctgcgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgctgataagtttaaactgccagaacatttctctggcctaactggccggtaccggcttcattttttccatttactgcagaggcttcattttttccatttactgcagaggcttcattttttccatttactgcagaactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttgataa

SEQ ID NO: 170 - DNA, 3487 bp, synthetic construct
  source 1..3487: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtggctatgccatgagctgggtccgccaggctccagggaaggggctggagtgggtctcaaccattagtagtggcggaacctacatatactaccccgactcagtgaagggccgattcaccatctccagagacaacgccaagaactcactgtatctgcaaatgaacagcctgagagccgaggacacggccgtgtattactgtgcgagacttgggggggataattactacgaatacttcgatgtctggggcaaagggaccacggtcaccgtctcctccggcggtggcggatccggcggtggcggatccggcggtggcggatccgacattgtgctgacccagtctccagcctccttggccgtgtctccaggacagagggccaccatcacctgcagagccagtaagagtgtcagtaccagcggatactcctacatgcactggtatcagcagaaaccaggacaacctcctaaactcctgatttacctggcatccaatctggagagcggggtcccagccaggttcagcggcagtgggtctgggaccgatttcaccctcacaattaatcctgtggaagctaatgatactgcaaattattactgtcagcacagtagggagctgcctttcacattcggcggagggaccaaggtggagatcaaacgaactacaacaacccctgcccccagacctcctaccccagcccctacaattgccagccagcctctgagcctgaggcccgaggcttgtagacctgctgctggcggagccgtgcacaccagaggactggatttcgcctgcgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgctgataagtttaaactgccagaacatttctctggcctaactggccggtaccggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttgataa

SEQ ID NO: 171 - DNA, 3397 bp, synthetic construct
  source 1..3397: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtggctatgccatgagctgggtccgccaggctccagggaaggggctggagtgggtctcaaccattagtagtggcggaacctacatatactaccccgactcagtgaagggccgattcaccatctccagagacaacgccaagaactcactgtatctgcaaatgaacagcctgagagccgaggacacggccgtgtattactgtgcgagacttgggggggataattactacgaatacttcgatgtctggggcaaagggaccacggtcaccgtctcctccggcggtggcggatccggcggtggcggatccggcggtggcggatccgacattgtgctgacccagtctccagcctccttggccgtgtctccaggacagagggccaccatcacctgcagagccagtaagagtgtcagtaccagcggatactcctacatgcactggtatcagcagaaaccaggacaacctcctaaactcctgatttacctggcatccaatctggagagcggggtcccagccaggttcagcggcagtgggtctgggaccgatttcaccctcacaattaatcctgtggaagctaatgatactgcaaattattactgtcagcacagtagggagctgcctttcacattcggcggagggaccaaggtggagatcaaacgaactacaacaacccctgcccccagacctcctaccccagcccctacaattgccagccagcctctgagcctgaggcccgaggcttgtagacctgctgctggcggagccgtgcacaccagaggactggatttcgcctgcgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgctgataagtttaaactgccagaacatttctctggcctaactggccggtaccggaggaaaaactgtttcatacagaaggcgtggaggaaaaactgtttcatacagaaggcgtggaggaaaaactgtttcatacagaaggcgtactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttgataa

SEQ ID NO: 172 - DNA, 3451 bp, synthetic construct
  source 1..3451: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtaggtatggcatgagctgggtccgccaggctccagggaagaggctggagtgggtctcaaccattagtggcggaggcacctacatatactacccagactcagtgaagggccgattcaccatctccagagacaacgccaagaacaccctgtatctgcaaatgaacagcctgagagccgaggacacggctgtgtattactgtaccagagataactatggccgcaactatgattatggcatggattattggggccagggcaccctggtgaccgtgagcagcggtggtggcggttccggcggtggcggttccggtggtggcggttccgaaattgtgttgacacagtctccagccaccctgtctttgtctccaggggaaagagccaccctcacctgcagcgccaccagcagtgttagctacatccactggtatcaacagaggcctggccagagccccaggctcctcatctatagcacctccaacctggccagcggcatcccagccaggttcagtggcagtgggtctgggagcgactacactctcaccatcagcagcctagagcctgaagattttgcagtttattactgtcagcagcgtagcagctcccctttcacctttggcagcggcaccaaagtggaaattaaaacaacaacccctgcccccagacctcctaccccagcccctacaattgccagccagcctctgagcctgaggcccgaggcttgtagacctgctgctggcggagccgtgcacaccagaggactggatttcgcctgcgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgctgataagtttaaactgccagaacatttctctggcctaactggccggtaccggcttcattttttccatttactgcagaggcttcattttttccatttactgcagaggcttcattttttccatttactgcagaggcttcattttttccatttactgcagaggcttcattttttccatttactgcagaggcttcattttttccatttactgcagaactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttgataa

SEQ ID NO: 173 - DNA, 3370 bp, synthetic construct
  source 1..3370: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtaggtatggcatgagctgggtccgccaggctccagggaagaggctggagtgggtctcaaccattagtggcggaggcacctacatatactacccagactcagtgaagggccgattcaccatctccagagacaacgccaagaacaccctgtatctgcaaatgaacagcctgagagccgaggacacggctgtgtattactgtaccagagataactatggccgcaactatgattatggcatggattattggggccagggcaccctggtgaccgtgagcagcggtggtggcggttccggcggtggcggttccggtggtggcggttccgaaattgtgttgacacagtctccagccaccctgtctttgtctccaggggaaagagccaccctcacctgcagcgccaccagcagtgttagctacatccactggtatcaacagaggcctggccagagccccaggctcctcatctatagcacctccaacctggccagcggcatcccagccaggttcagtggcagtgggtctgggagcgactacactctcaccatcagcagcctagagcctgaagattttgcagtttattactgtcagcagcgtagcagctcccctttcacctttggcagcggcaccaaagtggaaattaaaacaacaacccctgcccccagacctcctaccccagcccctacaattgccagccagcctctgagcctgaggcccgaggcttgtagacctgctgctggcggagccgtgcacaccagaggactggatttcgcctgcgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgctgataagtttaaactgccagaacatttctctggcctaactggccggtaccggcttcattttttccatttactgcagaggcttcattttttccatttactgcagaggcttcattttttccatttactgcagaactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttgataa

SEQ ID NO: 174 - DNA, 3469 bp, synthetic construct
  source 1..3469: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtaggtatggcatgagctgggtccgccaggctccagggaagaggctggagtgggtctcaaccattagtggcggaggcacctacatatactacccagactcagtgaagggccgattcaccatctccagagacaacgccaagaacaccctgtatctgcaaatgaacagcctgagagccgaggacacggctgtgtattactgtaccagagataactatggccgcaactatgattatggcatggattattggggccagggcaccctggtgaccgtgagcagcggtggtggcggttccggcggtggcggttccggtggtggcggttccgaaattgtgttgacacagtctccagccaccctgtctttgtctccaggggaaagagccaccctcacctgcagcgccaccagcagtgttagctacatccactggtatcaacagaggcctggccagagccccaggctcctcatctatagcacctccaacctggccagcggcatcccagccaggttcagtggcagtgggtctgggagcgactacactctcaccatcagcagcctagagcctgaagattttgcagtttattactgtcagcagcgtagcagctcccctttcacctttggcagcggcaccaaagtggaaattaaaacaacaacccctgcccccagacctcctaccccagcccctacaattgccagccagcctctgagcctgaggcccgaggcttgtagacctgctgctggcggagccgtgcacaccagaggactggatttcgcctgcgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgctgataagtttaaactgccagaacatttctctggcctaactggccggtaccggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttgataa

SEQ ID NO: 175 - DNA, 3379 bp, synthetic construct
  source 1..3379: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtaggtatggcatgagctgggtccgccaggctccagggaagaggctggagtgggtctcaaccattagtggcggaggcacctacatatactacccagactcagtgaagggccgattcaccatctccagagacaacgccaagaacaccctgtatctgcaaatgaacagcctgagagccgaggacacggctgtgtattactgtaccagagataactatggccgcaactatgattatggcatggattattggggccagggcaccctggtgaccgtgagcagcggtggtggcggttccggcggtggcggttccggtggtggcggttccgaaattgtgttgacacagtctccagccaccctgtctttgtctccaggggaaagagccaccctcacctgcagcgccaccagcagtgttagctacatccactggtatcaacagaggcctggccagagccccaggctcctcatctatagcacctccaacctggccagcggcatcccagccaggttcagtggcagtgggtctgggagcgactacactctcaccatcagcagcctagagcctgaagattttgcagtttattactgtcagcagcgtagcagctcccctttcacctttggcagcggcaccaaagtggaaattaaaacaacaacccctgcccccagacctcctaccccagcccctacaattgccagccagcctctgagcctgaggcccgaggcttgtagacctgctgctggcggagccgtgcacaccagaggactggatttcgcctgcgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgctgataagtttaaactgccagaacatttctctggcctaactggccggtaccggaggaaaaactgtttcatacagaaggcgtggaggaaaaactgtttcatacagaaggcgtggaggaaaaactgtttcatacagaaggcgtactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttgataa

SEQ ID NO: 176 - DNA, 5329 bp, synthetic construct
  source 1..5329: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtggctatgccatgagctgggtccgccaggctccagggaaggggctggagtgggtctcaaccattagtagtggcggaacctacatatactaccccgactcagtgaagggccgattcaccatctccagagacaacgccaagaactcactgtatctgcaaatgaacagcctgagagccgaggacacggccgtgtattactgtgcgagacttgggggggataattactacgaatacttcgatgtctggggcaaagggaccacggtcaccgtctcctccggcggtggcggatccggcggtggcggatccggcggtggcggatccgacattgtgctgacccagtctccagcctccttggccgtgtctccaggacagagggccaccatcacctgcagagccagtaagagtgtcagtaccagcggatactcctacatgcactggtatcagcagaaaccaggacaacctcctaaactcctgatttacctggcatccaatctggagagcggggtcccagccaggttcagcggcagtgggtctgggaccgatttcaccctcacaattaatcctgtggaagctaatgatactgcaaattattactgtcagcacagtagggagctgcctttcacattcggcggagggaccaaggtggagatcaaacgaactacaacaacccctgcccccagacctcctaccccagcccctacaattgccagccagcctctgagcctgaggcccgaggcttgtagacctgctgctggcggagccgtgcacaccagaggactggatttcgcctgcgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgctgataagtttaaactgccagaacatttctctggcctaactggccggtaccggcttcattttttccatttactgcagaggcttcattttttccatttactgcagaggcttcattttttccatttactgcagaggcttcattttttccatttactgcagaggcttcattttttccatttactgcagaggcttcattttttccatttactgcagaactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtctggaagcggtgagggaagaggtagcctgctcacgtgcggtgatgtggaggagaaccctgggcctatgctctgtttgggctggatatttctttggcttgttgcaggagagcgaattaaaggatttaatatttcaggttgttccacaaaaaaactcctttggacatattctacaaggagtgaagaggaatttgtcttattttgtgatttaccagagccacagaaatcacatttctgccacagaaatcgactctcaccaaaacaagtccctgagcacctgcccttcatgggtagtaacgacctatctgatgtccaatggtaccaacaaccttcgaatggagatccattagaggacattaggaaaagctatcctcacatcattcaggacaaatgtacccttcactttttgaccccaggggtgaataattctgggtcatatatttgtagacccaagatgattaagagcccctatgatgtagcctgttgtgtcaagatgattttagaagttaagccccagacaaatgcatcctgtgagtattccgcatcacataagcaagacctacttcttgggagcactggctctatttcttgccccagtctcagctgccaaagtgatgcacaaagtccagcggtaacctggtacaagaatggaaaactcctctctgtggaaaggagcaaccgaatcgtagtggatgaagtttatgactatcaccagggcacatatgtatgtgattacactcagtcggatactgtgagttcgtggacagtcagagctgttgttcaagtgagaaccattgtgggagacactaaactcaaaccagatattctggatcctgtcgaggacacactggaagtagaacttggaaagcctttaactattagctgcaaagcacgatttggctttgaaagggtctttaaccctgtcataaaatggtacatcaaagattctgacctagagtgggaagtctcagtacctgaggcgaaaagtattaaatccactttaaaggatgaaatcattgagcgtaatatcatcttggaaaaagtcactcagcgtgatcttcgcaggaagtttgtttgctttgtccagaactccattggaaacacaacccagtccgtccaactgaaagaaaagagaggagtggtgctcctgtacatcctgcttggcaccatcgggaccctggtggccgtgctggcggcgagtgccctcctctacaggcactggattgaaatagtgctgctgtaccggacctaccagagcaaggatcagacgcttggggataaaaaggattttgatgctttcgtatcctatgcaaaatggagctcttttccaagtgaggccacttcatctctgagtgaagaacacttggccctgagcctatttcctgatgttttagaaaacaaatatggatatagcctgtgtttgcttgaaagagatgtggctccaggaggagtgtatgcagaagacattgtgagcattattaagagaagcagaagaggaatatttatcttgagccccaactatgtcaatggacccagtatctttgaactacaagcagcagtgaatcttgccttggatgatcaaacactgaaactcattttaattaagttctgttacttccaagagccagagtctctacctcatctcgtgaaaaaagctctcagggttttgcccacagttacttggagaggcttaaaatcagttcctcccaattctaggttctgggccaaaatgcgctaccacatgcctgtgaaaaactctcagggattcacgtggaaccagctcagaattacctctaggatttttcagtggaaaggactcagtagaacagaaaccactgggaggagctcccagcctaaggaatggtgataa

SEQ ID NO: 177 - DNA, 5248 bp, synthetic construct
  source 1..5248: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtggctatgccatgagctgggtccgccaggctccagggaaggggctggagtgggtctcaaccattagtagtggcggaacctacatatactaccccgactcagtgaagggccgattcaccatctccagagacaacgccaagaactcactgtatctgcaaatgaacagcctgagagccgaggacacggccgtgtattactgtgcgagacttgggggggataattactacgaatacttcgatgtctggggcaaagggaccacggtcaccgtctcctccggcggtggcggatccggcggtggcggatccggcggtggcggatccgacattgtgctgacccagtctccagcctccttggccgtgtctccaggacagagggccaccatcacctgcagagccagtaagagtgtcagtaccagcggatactcctacatgcactggtatcagcagaaaccaggacaacctcctaaactcctgatttacctggcatccaatctggagagcggggtcccagccaggttcagcggcagtgggtctgggaccgatttcaccctcacaattaatcctgtggaagctaatgatactgcaaattattactgtcagcacagtagggagctgcctttcacattcggcggagggaccaaggtggagatcaaacgaactacaacaacccctgcccccagacctcctaccccagcccctacaattgccagccagcctctgagcctgaggcccgaggcttgtagacctgctgctggcggagccgtgcacaccagaggactggatttcgcctgcgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgctgataagtttaaactgccagaacatttctctggcctaactggccggtaccggcttcattttttccatttactgcagaggcttcattttttccatttactgcagaggcttcattttttccatttactgcagaactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtctggaagcggtgagggaagaggtagcctgctcacgtgcggtgatgtggaggagaaccctgggcctatgctctgtttgggctggatatttctttggcttgttgcaggagagcgaattaaaggatttaatatttcaggttgttccacaaaaaaactcctttggacatattctacaaggagtgaagaggaatttgtcttattttgtgatttaccagagccacagaaatcacatttctgccacagaaatcgactctcaccaaaacaagtccctgagcacctgcccttcatgggtagtaacgacctatctgatgtccaatggtaccaacaaccttcgaatggagatccattagaggacattaggaaaagctatcctcacatcattcaggacaaatgtacccttcactttttgaccccaggggtgaataattctgggtcatatatttgtagacccaagatgattaagagcccctatgatgtagcctgttgtgtcaagatgattttagaagttaagccccagacaaatgcatcctgtgagtattccgcatcacataagcaagacctacttcttgggagcactggctctatttcttgccccagtctcagctgccaaagtgatgcacaaagtccagcggtaacctggtacaagaatggaaaactcctctctgtggaaaggagcaaccgaatcgtagtggatgaagtttatgactatcaccagggcacatatgtatgtgattacactcagtcggatactgtgagttcgtggacagtcagagctgttgttcaagtgagaaccattgtgggagacactaaactcaaaccagatattctggatcctgtcgaggacacactggaagtagaacttggaaagcctttaactattagctgcaaagcacgatttggctttgaaagggtctttaaccctgtcataaaatggtacatcaaagattctgacctagagtgggaagtctcagtacctgaggcgaaaagtattaaatccactttaaaggatgaaatcattgagcgtaatatcatcttggaaaaagtcactcagcgtgatcttcgcaggaagtttgtttgctttgtccagaactccattggaaacacaacccagtccgtccaactgaaagaaaagagaggagtggtgctcctgtacatcctgcttggcaccatcgggaccctggtggccgtgctggcggcgagtgccctcctctacaggcactggattgaaatagtgctgctgtaccggacctaccagagcaaggatcagacgcttggggataaaaaggattttgatgctttcgtatcctatgcaaaatggagctcttttccaagtgaggccacttcatctctgagtgaagaacacttggccctgagcctatttcctgatgttttagaaaacaaatatggatatagcctgtgtttgcttgaaagagatgtggctccaggaggagtgtatgcagaagacattgtgagcattattaagagaagcagaagaggaatatttatcttgagccccaactatgtcaatggacccagtatctttgaactacaagcagcagtgaatcttgccttggatgatcaaacactgaaactcattttaattaagttctgttacttccaagagccagagtctctacctcatctcgtgaaaaaagctctcagggttttgcccacagttacttggagaggcttaaaatcagttcctcccaattctaggttctgggccaaaatgcgctaccacatgcctgtgaaaaactctcagggattcacgtggaaccagctcagaattacctctaggatttttcagtggaaaggactcagtagaacagaaaccactgggaggagctcccagcctaaggaatggtgataa

SEQ ID NO: 178 - DNA, 5347 bp, synthetic construct
  source 1..5347: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtggctatgccatgagctgggtccgccaggctccagggaaggggctggagtgggtctcaaccattagtagtggcggaacctacatatactaccccgactcagtgaagggccgattcaccatctccagagacaacgccaagaactcactgtatctgcaaatgaacagcctgagagccgaggacacggccgtgtattactgtgcgagacttgggggggataattactacgaatacttcgatgtctggggcaaagggaccacggtcaccgtctcctccggcggtggcggatccggcggtggcggatccggcggtggcggatccgacattgtgctgacccagtctccagcctccttggccgtgtctccaggacagagggccaccatcacctgcagagccagtaagagtgtcagtaccagcggatactcctacatgcactggtatcagcagaaaccaggacaacctcctaaactcctgatttacctggcatccaatctggagagcggggtcccagccaggttcagcggcagtgggtctgggaccgatttcaccctcacaattaatcctgtggaagctaatgatactgcaaattattactgtcagcacagtagggagctgcctttcacattcggcggagggaccaaggtggagatcaaacgaactacaacaacccctgcccccagacctcctaccccagcccctacaattgccagccagcctctgagcctgaggcccgaggcttgtagacctgctgctggcggagccgtgcacaccagaggactggatttcgcctgcgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgctgataagtttaaactgccagaacatttctctggcctaactggccggtaccggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtctggaagcggtgagggaagaggtagcctgctcacgtgcggtgatgtggaggagaaccctgggcctatgctctgtttgggctggatatttctttggcttgttgcaggagagcgaattaaaggatttaatatttcaggttgttccacaaaaaaactcctttggacatattctacaaggagtgaagaggaatttgtcttattttgtgatttaccagagccacagaaatcacatttctgccacagaaatcgactctcaccaaaacaagtccctgagcacctgcccttcatgggtagtaacgacctatctgatgtccaatggtaccaacaaccttcgaatggagatccattagaggacattaggaaaagctatcctcacatcattcaggacaaatgtacccttcactttttgaccccaggggtgaataattctgggtcatatatttgtagacccaagatgattaagagcccctatgatgtagcctgttgtgtcaagatgattttagaagttaagccccagacaaatgcatcctgtgagtattccgcatcacataagcaagacctacttcttgggagcactggctctatttcttgccccagtctcagctgccaaagtgatgcacaaagtccagcggtaacctggtacaagaatggaaaactcctctctgtggaaaggagcaaccgaatcgtagtggatgaagtttatgactatcaccagggcacatatgtatgtgattacactcagtcggatactgtgagttcgtggacagtcagagctgttgttcaagtgagaaccattgtgggagacactaaactcaaaccagatattctggatcctgtcgaggacacactggaagtagaacttggaaagcctttaactattagctgcaaagcacgatttggctttgaaagggtctttaaccctgtcataaaatggtacatcaaagattctgacctagagtgggaagtctcagtacctgaggcgaaaagtattaaatccactttaaaggatgaaatcattgagcgtaatatcatcttggaaaaagtcactcagcgtgatcttcgcaggaagtttgtttgctttgtccagaactccattggaaacacaacccagtccgtccaactgaaagaaaagagaggagtggtgctcctgtacatcctgcttggcaccatcgggaccctggtggccgtgctggcggcgagtgccctcctctacaggcactggattgaaatagtgctgctgtaccggacctaccagagcaaggatcagacgcttggggataaaaaggattttgatgctttcgtatcctatgcaaaatggagctcttttccaagtgaggccacttcatctctgagtgaagaacacttggccctgagcctatttcctgatgttttagaaaacaaatatggatatagcctgtgtttgcttgaaagagatgtggctccaggaggagtgtatgcagaagacattgtgagcattattaagagaagcagaagaggaatatttatcttgagccccaactatgtcaatggacccagtatctttgaactacaagcagcagtgaatcttgccttggatgatcaaacactgaaactcattttaattaagttctgttacttccaagagccagagtctctacctcatctcgtgaaaaaagctctcagggttttgcccacagttacttggagaggcttaaaatcagttcctcccaattctaggttctgggccaaaatgcgctaccacatgcctgtgaaaaactctcagggattcacgtggaaccagctcagaattacctctaggatttttcagtggaaaggactcagtagaacagaaaccactgggaggagctcccagcctaaggaatggtgataa

SEQ ID NO: 179 - DNA, 5257 bp, synthetic construct
  source 1..5257: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtggctatgccatgagctgggtccgccaggctccagggaaggggctggagtgggtctcaaccattagtagtggcggaacctacatatactaccccgactcagtgaagggccgattcaccatctccagagacaacgccaagaactcactgtatctgcaaatgaacagcctgagagccgaggacacggccgtgtattactgtgcgagacttgggggggataattactacgaatacttcgatgtctggggcaaagggaccacggtcaccgtctcctccggcggtggcggatccggcggtggcggatccggcggtggcggatccgacattgtgctgacccagtctccagcctccttggccgtgtctccaggacagagggccaccatcacctgcagagccagtaagagtgtcagtaccagcggatactcctacatgcactggtatcagcagaaaccaggacaacctcctaaactcctgatttacctggcatccaatctggagagcggggtcccagccaggttcagcggcagtgggtctgggaccgatttcaccctcacaattaatcctgtggaagctaatgatactgcaaattattactgtcagcacagtagggagctgcctttcacattcggcggagggaccaaggtggagatcaaacgaactacaacaacccctgcccccagacctcctaccccagcccctacaattgccagccagcctctgagcctgaggcccgaggcttgtagacctgctgctggcggagccgtgcacaccagaggactggatttcgcctgcgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgctgataagtttaaactgccagaacatttctctggcctaactggccggtaccggaggaaaaactgtttcatacagaaggcgtggaggaaaaactgtttcatacagaaggcgtggaggaaaaactgtttcatacagaaggcgtactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtctggaagcggtgagggaagaggtagcctgctcacgtgcggtgatgtggaggagaaccctgggcctatgctctgtttgggctggatatttctttggcttgttgcaggagagcgaattaaaggatttaatatttcaggttgttccacaaaaaaactcctttggacatattctacaaggagtgaagaggaatttgtcttattttgtgatttaccagagccacagaaatcacatttctgccacagaaatcgactctcaccaaaacaagtccctgagcacctgcccttcatgggtagtaacgacctatctgatgtccaatggtaccaacaaccttcgaatggagatccattagaggacattaggaaaagctatcctcacatcattcaggacaaatgtacccttcactttttgaccccaggggtgaataattctgggtcatatatttgtagacccaagatgattaagagcccctatgatgtagcctgttgtgtcaagatgattttagaagttaagccccagacaaatgcatcctgtgagtattccgcatcacataagcaagacctacttcttgggagcactggctctatttcttgccccagtctcagctgccaaagtgatgcacaaagtccagcggtaacctggtacaagaatggaaaactcctctctgtggaaaggagcaaccgaatcgtagtggatgaagtttatgactatcaccagggcacatatgtatgtgattacactcagtcggatactgtgagttcgtggacagtcagagctgttgttcaagtgagaaccattgtgggagacactaaactcaaaccagatattctggatcctgtcgaggacacactggaagtagaacttggaaagcctttaactattagctgcaaagcacgatttggctttgaaagggtctttaaccctgtcataaaatggtacatcaaagattctgacctagagtgggaagtctcagtacctgaggcgaaaagtattaaatccactttaaaggatgaaatcattgagcgtaatatcatcttggaaaaagtcactcagcgtgatcttcgcaggaagtttgtttgctttgtccagaactccattggaaacacaacccagtccgtccaactgaaagaaaagagaggagtggtgctcctgtacatcctgcttggcaccatcgggaccctggtggccgtgctggcggcgagtgccctcctctacaggcactggattgaaatagtgctgctgtaccggacctaccagagcaaggatcagacgcttggggataaaaaggattttgatgctttcgtatcctatgcaaaatggagctcttttccaagtgaggccacttcatctctgagtgaagaacacttggccctgagcctatttcctgatgttttagaaaacaaatatggatatagcctgtgtttgcttgaaagagatgtggctccaggaggagtgtatgcagaagacattgtgagcattattaagagaagcagaagaggaatatttatcttgagccccaactatgtcaatggacccagtatctttgaactacaagcagcagtgaatcttgccttggatgatcaaacactgaaactcattttaattaagttctgttacttccaagagccagagtctctacctcatctcgtgaaaaaagctctcagggttttgcccacagttacttggagaggcttaaaatcagttcctcccaattctaggttctgggccaaaatgcgctaccacatgcctgtgaaaaactctcagggattcacgtggaaccagctcagaattacctctaggatttttcagtggaaaggactcagtagaacagaaaccactgggaggagctcccagcctaaggaatggtgataa

SEQ ID NO: 180 - DNA, 5311 bp, synthetic construct
  source 1..5311: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtaggtatggcatgagctgggtccgccaggctccagggaagaggctggagtgggtctcaaccattagtggcggaggcacctacatatactacccagactcagtgaagggccgattcaccatctccagagacaacgccaagaacaccctgtatctgcaaatgaacagcctgagagccgaggacacggctgtgtattactgtaccagagataactatggccgcaactatgattatggcatggattattggggccagggcaccctggtgaccgtgagcagcggtggtggcggttccggcggtggcggttccggtggtggcggttccgaaattgtgttgacacagtctccagccaccctgtctttgtctccaggggaaagagccaccctcacctgcagcgccaccagcagtgttagctacatccactggtatcaacagaggcctggccagagccccaggctcctcatctatagcacctccaacctggccagcggcatcccagccaggttcagtggcagtgggtctgggagcgactacactctcaccatcagcagcctagagcctgaagattttgcagtttattactgtcagcagcgtagcagctcccctttcacctttggcagcggcaccaaagtggaaattaaaacaacaacccctgcccccagacctcctaccccagcccctacaattgccagccagcctctgagcctgaggcccgaggcttgtagacctgctgctggcggagccgtgcacaccagaggactggatttcgcctgcgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgctgataagtttaaactgccagaacatttctctggcctaactggccggtaccggcttcattttttccatttactgcagaggcttcattttttccatttactgcagaggcttcattttttccatttactgcagaggcttcattttttccatttactgcagaggcttcattttttccatttactgcagaggcttcattttttccatttactgcagaactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtctggaagcggtgagggaagaggtagcctgctcacgtgcggtgatgtggaggagaaccctgggcctatgctctgtttgggctggatatttctttggcttgttgcaggagagcgaattaaaggatttaatatttcaggttgttccacaaaaaaactcctttggacatattctacaaggagtgaagaggaatttgtcttattttgtgatttaccagagccacagaaatcacatttctgccacagaaatcgactctcaccaaaacaagtccctgagcacctgcccttcatgggtagtaacgacctatctgatgtccaatggtaccaacaaccttcgaatggagatccattagaggacattaggaaaagctatcctcacatcattcaggacaaatgtacccttcactttttgaccccaggggtgaataattctgggtcatatatttgtagacccaagatgattaagagcccctatgatgtagcctgttgtgtcaagatgattttagaagttaagccccagacaaatgcatcctgtgagtattccgcatcacataagcaagacctacttcttgggagcactggctctatttcttgccccagtctcagctgccaaagtgatgcacaaagtccagcggtaacctggtacaagaatggaaaactcctctctgtggaaaggagcaaccgaatcgtagtggatgaagtttatgactatcaccagggcacatatgtatgtgattacactcagtcggatactgtgagttcgtggacagtcagagctgttgttcaagtgagaaccattgtgggagacactaaactcaaaccagatattctggatcctgtcgaggacacactggaagtagaacttggaaagcctttaactattagctgcaaagcacgatttggctttgaaagggtctttaaccctgtcataaaatggtacatcaaagattctgacctagagtgggaagtctcagtacctgaggcgaaaagtattaaatccactttaaaggatgaaatcattgagcgtaatatcatcttggaaaaagtcactcagcgtgatcttcgcaggaagtttgtttgctttgtccagaactccattggaaacacaacccagtccgtccaactgaaagaaaagagaggagtggtgctcctgtacatcctgcttggcaccatcgggaccctggtggccgtgctggcggcgagtgccctcctctacaggcactggattgaaatagtgctgctgtaccggacctaccagagcaaggatcagacgcttggggataaaaaggattttgatgctttcgtatcctatgcaaaatggagctcttttccaagtgaggccacttcatctctgagtgaagaacacttggccctgagcctatttcctgatgttttagaaaacaaatatggatatagcctgtgtttgcttgaaagagatgtggctccaggaggagtgtatgcagaagacattgtgagcattattaagagaagcagaagaggaatatttatcttgagccccaactatgtcaatggacccagtatctttgaactacaagcagcagtgaatcttgccttggatgatcaaacactgaaactcattttaattaagttctgttacttccaagagccagagtctctacctcatctcgtgaaaaaagctctcagggttttgcccacagttacttggagaggcttaaaatcagttcctcccaattctaggttctgggccaaaatgcgctaccacatgcctgtgaaaaactctcagggattcacgtggaaccagctcagaattacctctaggatttttcagtggaaaggactcagtagaacagaaaccactgggaggagctcccagcctaaggaatggtgataa

SEQ ID NO: 181 - DNA, 5230 bp, synthetic construct
  source 1..5230: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtaggtatggcatgagctgggtccgccaggctccagggaagaggctggagtgggtctcaaccattagtggcggaggcacctacatatactacccagactcagtgaagggccgattcaccatctccagagacaacgccaagaacaccctgtatctgcaaatgaacagcctgagagccgaggacacggctgtgtattactgtaccagagataactatggccgcaactatgattatggcatggattattggggccagggcaccctggtgaccgtgagcagcggtggtggcggttccggcggtggcggttccggtggtggcggttccgaaattgtgttgacacagtctccagccaccctgtctttgtctccaggggaaagagccaccctcacctgcagcgccaccagcagtgttagctacatccactggtatcaacagaggcctggccagagccccaggctcctcatctatagcacctccaacctggccagcggcatcccagccaggttcagtggcagtgggtctgggagcgactacactctcaccatcagcagcctagagcctgaagattttgcagtttattactgtcagcagcgtagcagctcccctttcacctttggcagcggcaccaaagtggaaattaaaacaacaacccctgcccccagacctcctaccccagcccctacaattgccagccagcctctgagcctgaggcccgaggcttgtagacctgctgctggcggagccgtgcacaccagaggactggatttcgcctgcgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgctgataagtttaaactgccagaacatttctctggcctaactggccggtaccggcttcattttttccatttactgcagaggcttcattttttccatttactgcagaggcttcattttttccatttactgcagaactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtctggaagcggtgagggaagaggtagcctgctcacgtgcggtgatgtggaggagaaccctgggcctatgctctgtttgggctggatatttctttggcttgttgcaggagagcgaattaaaggatttaatatttcaggttgttccacaaaaaaactcctttggacatattctacaaggagtgaagaggaatttgtcttattttgtgatttaccagagccacagaaatcacatttctgccacagaaatcgactctcaccaaaacaagtccctgagcacctgcccttcatgggtagtaacgacctatctgatgtccaatggtaccaacaaccttcgaatggagatccattagaggacattaggaaaagctatcctcacatcattcaggacaaatgtacccttcactttttgaccccaggggtgaataattctgggtcatatatttgtagacccaagatgattaagagcccctatgatgtagcctgttgtgtcaagatgattttagaagttaagccccagacaaatgcatcctgtgagtattccgcatcacataagcaagacctacttcttgggagcactggctctatttcttgccccagtctcagctgccaaagtgatgcacaaagtccagcggtaacctggtacaagaatggaaaactcctctctgtggaaaggagcaaccgaatcgtagtggatgaagtttatgactatcaccagggcacatatgtatgtgattacactcagtcggatactgtgagttcgtggacagtcagagctgttgttcaagtgagaaccattgtgggagacactaaactcaaaccagatattctggatcctgtcgaggacacactggaagtagaacttggaaagcctttaactattagctgcaaagcacgatttggctttgaaagggtctttaaccctgtcataaaatggtacatcaaagattctgacctagagtgggaagtctcagtacctgaggcgaaaagtattaaatccactttaaaggatgaaatcattgagcgtaatatcatcttggaaaaagtcactcagcgtgatcttcgcaggaagtttgtttgctttgtccagaactccattggaaacacaacccagtccgtccaactgaaagaaaagagaggagtggtgctcctgtacatcctgcttggcaccatcgggaccctggtggccgtgctggcggcgagtgccctcctctacaggcactggattgaaatagtgctgctgtaccggacctaccagagcaaggatcagacgcttggggataaaaaggattttgatgctttcgtatcctatgcaaaatggagctcttttccaagtgaggccacttcatctctgagtgaagaacacttggccctgagcctatttcctgatgttttagaaaacaaatatggatatagcctgtgtttgcttgaaagagatgtggctccaggaggagtgtatgcagaagacattgtgagcattattaagagaagcagaagaggaatatttatcttgagccccaactatgtcaatggacccagtatctttgaactacaagcagcagtgaatcttgccttggatgatcaaacactgaaactcattttaattaagttctgttacttccaagagccagagtctctacctcatctcgtgaaaaaagctctcagggttttgcccacagttacttggagaggcttaaaatcagttcctcccaattctaggttctgggccaaaatgcgctaccacatgcctgtgaaaaactctcagggattcacgtggaaccagctcagaattacctctaggatttttcagtggaaaggactcagtagaacagaaaccactgggaggagctcccagcctaaggaatggtgataa

SEQ ID NO: 182 - DNA, 5329 bp, synthetic construct
  source 1..5329: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtaggtatggcatgagctgggtccgccaggctccagggaagaggctggagtgggtctcaaccattagtggcggaggcacctacatatactacccagactcagtgaagggccgattcaccatctccagagacaacgccaagaacaccctgtatctgcaaatgaacagcctgagagccgaggacacggctgtgtattactgtaccagagataactatggccgcaactatgattatggcatggattattggggccagggcaccctggtgaccgtgagcagcggtggtggcggttccggcggtggcggttccggtggtggcggttccgaaattgtgttgacacagtctccagccaccctgtctttgtctccaggggaaagagccaccctcacctgcagcgccaccagcagtgttagctacatccactggtatcaacagaggcctggccagagccccaggctcctcatctatagcacctccaacctggccagcggcatcccagccaggttcagtggcagtgggtctgggagcgactacactctcaccatcagcagcctagagcctgaagattttgcagtttattactgtcagcagcgtagcagctcccctttcacctttggcagcggcaccaaagtggaaattaaaacaacaacccctgcccccagacctcctaccccagcccctacaattgccagccagcctctgagcctgaggcccgaggcttgtagacctgctgctggcggagccgtgcacaccagaggactggatttcgcctgcgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgctgataagtttaaactgccagaacatttctctggcctaactggccggtaccggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtctggaagcggtgagggaagaggtagcctgctcacgtgcggtgatgtggaggagaaccctgggcctatgctctgtttgggctggatatttctttggcttgttgcaggagagcgaattaaaggatttaatatttcaggttgttccacaaaaaaactcctttggacatattctacaaggagtgaagaggaatttgtcttattttgtgatttaccagagccacagaaatcacatttctgccacagaaatcgactctcaccaaaacaagtccctgagcacctgcccttcatgggtagtaacgacctatctgatgtccaatggtaccaacaaccttcgaatggagatccattagaggacattaggaaaagctatcctcacatcattcaggacaaatgtacccttcactttttgaccccaggggtgaataattctgggtcatatatttgtagacccaagatgattaagagcccctatgatgtagcctgttgtgtcaagatgattttagaagttaagccccagacaaatgcatcctgtgagtattccgcatcacataagcaagacctacttcttgggagcactggctctatttcttgccccagtctcagctgccaaagtgatgcacaaagtccagcggtaacctggtacaagaatggaaaactcctctctgtggaaaggagcaaccgaatcgtagtggatgaagtttatgactatcaccagggcacatatgtatgtgattacactcagtcggatactgtgagttcgtggacagtcagagctgttgttcaagtgagaaccattgtgggagacactaaactcaaaccagatattctggatcctgtcgaggacacactggaagtagaacttggaaagcctttaactattagctgcaaagcacgatttggctttgaaagggtctttaaccctgtcataaaatggtacatcaaagattctgacctagagtgggaagtctcagtacctgaggcgaaaagtattaaatccactttaaaggatgaaatcattgagcgtaatatcatcttggaaaaagtcactcagcgtgatcttcgcaggaagtttgtttgctttgtccagaactccattggaaacacaacccagtccgtccaactgaaagaaaagagaggagtggtgctcctgtacatcctgcttggcaccatcgggaccctggtggccgtgctggcggcgagtgccctcctctacaggcactggattgaaatagtgctgctgtaccggacctaccagagcaaggatcagacgcttggggataaaaaggattttgatgctttcgtatcctatgcaaaatggagctcttttccaagtgaggccacttcatctctgagtgaagaacacttggccctgagcctatttcctgatgttttagaaaacaaatatggatatagcctgtgtttgcttgaaagagatgtggctccaggaggagtgtatgcagaagacattgtgagcattattaagagaagcagaagaggaatatttatcttgagccccaactatgtcaatggacccagtatctttgaactacaagcagcagtgaatcttgccttggatgatcaaacactgaaactcattttaattaagttctgttacttccaagagccagagtctctacctcatctcgtgaaaaaagctctcagggttttgcccacagttacttggagaggcttaaaatcagttcctcccaattctaggttctgggccaaaatgcgctaccacatgcctgtgaaaaactctcagggattcacgtggaaccagctcagaattacctctaggatttttcagtggaaaggactcagtagaacagaaaccactgggaggagctcccagcctaaggaatggtgataa

SEQ ID NO: 183 - DNA, 5239 bp, synthetic construct
  source 1..5239: mol_type = other DNA
atggccttaccagtgaccgccttgctcctgccgctggccttgctgctccacgccgccaggccggaggtgcagctggtggagtctgggggaggcctggtcaagcctggggggtccctgagactctcctgtgcagcctctggattcaccttcagtaggtatggcatgagctgggtccgccaggctccagggaagaggctggagtgggtctcaaccattagtggcggaggcacctacatatactacccagactcagtgaagggccgattcaccatctccagagacaacgccaagaacaccctgtatctgcaaatgaacagcctgagagccgaggacacggctgtgtattactgtaccagagataactatggccgcaactatgattatggcatggattattggggccagggcaccctggtgaccgtgagcagcggtggtggcggttccggcggtggcggttccggtggtggcggttccgaaattgtgttgacacagtctccagccaccctgtctttgtctccaggggaaagagccaccctcacctgcagcgccaccagcagtgttagctacatccactggtatcaacagaggcctggccagagccccaggctcctcatctatagcacctccaacctggccagcggcatcccagccaggttcagtggcagtgggtctgggagcgactacactctcaccatcagcagcctagagcctgaagattttgcagtttattactgtcagcagcgtagcagctcccctttcacctttggcagcggcaccaaagtggaaattaaaacaacaacccctgcccccagacctcctaccccagcccctacaattgccagccagcctctgagcctgaggcccgaggcttgtagacctgctgctggcggagccgtgcacaccagaggactggatttcgcctgcgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgctgataagtttaaactgccagaacatttctctggcctaactggccggtaccggaggaaaaactgtttcatacagaaggcgtggaggaaaaactgtttcatacagaaggcgtggaggaaaaactgtttcatacagaaggcgtactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtctggaagcggtgagggaagaggtagcctgctcacgtgcggtgatgtggaggagaaccctgggcctatgctctgtttgggctggatatttctttggcttgttgcaggagagcgaattaaaggatttaatatttcaggttgttccacaaaaaaactcctttggacatattctacaaggagtgaagaggaatttgtcttattttgtgatttaccagagccacagaaatcacatttctgccacagaaatcgactctcaccaaaacaagtccctgagcacctgcccttcatgggtagtaacgacctatctgatgtccaatggtaccaacaaccttcgaatggagatccattagaggacattaggaaaagctatcctcacatcattcaggacaaatgtacccttcactttttgaccccaggggtgaataattctgggtcatatatttgtagacccaagatgattaagagcccctatgatgtagcctgttgtgtcaagatgattttagaagttaagccccagacaaatgcatcctgtgagtattccgcatcacataagcaagacctacttcttgggagcactggctctatttcttgccccagtctcagctgccaaagtgatgcacaaagtccagcggtaacctggtacaagaatggaaaactcctctctgtggaaaggagcaaccgaatcgtagtggatgaagtttatgactatcaccagggcacatatgtatgtgattacactcagtcggatactgtgagttcgtggacagtcagagctgttgttcaagtgagaaccattgtgggagacactaaactcaaaccagatattctggatcctgtcgaggacacactggaagtagaacttggaaagcctttaactattagctgcaaagcacgatttggctttgaaagggtctttaaccctgtcataaaatggtacatcaaagattctgacctagagtgggaagtctcagtacctgaggcgaaaagtattaaatccactttaaaggatgaaatcattgagcgtaatatcatcttggaaaaagtcactcagcgtgatcttcgcaggaagtttgtttgctttgtccagaactccattggaaacacaacccagtccgtccaactgaaagaaaagagaggagtggtgctcctgtacatcctgcttggcaccatcgggaccctggtggccgtgctggcggcgagtgccctcctctacaggcactggattgaaatagtgctgctgtaccggacctaccagagcaaggatcagacgcttggggataaaaaggattttgatgctttcgtatcctatgcaaaatggagctcttttccaagtgaggccacttcatctctgagtgaagaacacttggccctgagcctatttcctgatgttttagaaaacaaatatggatatagcctgtgtttgcttgaaagagatgtggctccaggaggagtgtatgcagaagacattgtgagcattattaagagaagcagaagaggaatatttatcttgagccccaactatgtcaatggacccagtatctttgaactacaagcagcagtgaatcttgccttggatgatcaaacactgaaactcattttaattaagttctgttacttccaagagccagagtctctacctcatctcgtgaaaaaagctctcagggttttgcccacagttacttggagaggcttaaaatcagttcctcccaattctaggttctgggccaaaatgcgctaccacatgcctgtgaaaaactctcagggattcacgtggaaccagctcagaattacctctaggatttttcagtggaaaggactcagtagaacagaaaccactgggaggagctcccagcctaaggaatggtgataa

SEQ ID NO: 184 - DNA, 3466 bp, synthetic construct
  source 1..3466: mol_type = other DNA
atggccttaccagtgaccgccttgctcctgccgctggccttgctgctccacgccgccaggccgcaggtgcagctggttgaatctggcggcggacttgtgaagcctggcggatctctgagactgagctgtgccgccagcggcttcacctttagcacatacgccatgagctggatcagacaggcccctggcaaaggcctggaatgggtggcgtctattggcagagccggcagcacctactacagcgactctgtgaagggcagattcaccatcagccgggacaacgccaagaacagcctgtacctgcagatgaactccctgagagccgaggacaccgccgtgtactattgtgccagaggacccatctacaacgactacgacgagttcgcctattggggccagggcacactggtcacagtttctagcggcggtggcggaagcggaggcggtggctccggtggcggaggcagcgaaattgtgctgacacagagccccgccacactgtcactttctccaggcgaaagagccacactgagctgcaagagcagccagagcgtgctgtactccagcaaccagaagaactacctggcctggtatcagcagaagcccggccaagctcctcggctgctgatctattgggccagcacaagagagagcggcatccctgccagattttctggcagcggctctggcaccgatttcaccctgaccataagcagcctggaacctgaggacttcgccgtgtattactgccaccagtacctgagcagcctgacctttggcggaggcaccaaggtggaaatcaagcggacaacaacccctgcccccagacctcctaccccagcccctacaattgccagccagcctctgagcctgaggcccgaggcttgtagacctgctgctggcggagccgtgcacaccagaggactggatttcgcctgcgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgctgataagtttaaactgccagaacatttctctggcctaactggccggtaccggcttcattttttccatttactgcagaggcttcattttttccatttactgcagaggcttcattttttccatttactgcagaggcttcattttttccatttactgcagaggcttcattttttccatttactgcagaggcttcattttttccatttactgcagaactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttgataa

SEQ ID NO: 185 - DNA, 3385 bp, synthetic construct
  source 1..3385: mol_type = other DNA
atggccttaccagtgaccgccttgctcctgccgctggccttgctgctccacgccgccaggccgcaggtgcagctggttgaatctggcggcggacttgtgaagcctggcggatctctgagactgagctgtgccgccagcggcttcacctttagcacatacgccatgagctggatcagacaggcccctggcaaaggcctggaatgggtggcgtctattggcagagccggcagcacctactacagcgactctgtgaagggcagattcaccatcagccgggacaacgccaagaacagcctgtacctgcagatgaactccctgagagccgaggacaccgccgtgtactattgtgccagaggacccatctacaacgactacgacgagttcgcctattggggccagggcacactggtcacagtttctagcggcggtggcggaagcggaggcggtggctccggtggcggaggcagcgaaattgtgctgacacagagccccgccacactgtcactttctccaggcgaaagagccacactgagctgcaagagcagccagagcgtgctgtactccagcaaccagaagaactacctggcctggtatcagcagaagcccggccaagctcctcggctgctgatctattgggccagcacaagagagagcggcatccctgccagattttctggcagcggctctggcaccgatttcaccctgaccataagcagcctggaacctgaggacttcgccgtgtattactgccaccagtacctgagcagcctgacctttggcggaggcaccaaggtggaaatcaagcggacaacaacccctgcccccagacctcctaccccagcccctacaattgccagccagcctctgagcctgaggcccgaggcttgtagacctgctgctggcggagccgtgcacaccagaggactggatttcgcctgcgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgctgataagtttaaactgccagaacatttctctggcctaactggccggtaccggcttcattttttccatttactgcagaggcttcattttttccatttactgcagaggcttcattttttccatttactgcagaactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttgataa

SEQ ID NO: 186 - DNA, 3484 bp, synthetic construct
  source 1..3484: mol_type = other DNA
atggccttaccagtgaccgccttgctcctgccgctggccttgctgctccacgccgccaggccgcaggtgcagctggttgaatctggcggcggacttgtgaagcctggcggatctctgagactgagctgtgccgccagcggcttcacctttagcacatacgccatgagctggatcagacaggcccctggcaaaggcctggaatgggtggcgtctattggcagagccggcagcacctactacagcgactctgtgaagggcagattcaccatcagccgggacaacgccaagaacagcctgtacctgcagatgaactccctgagagccgaggacaccgccgtgtactattgtgccagaggacccatctacaacgactacgacgagttcgcctattggggccagggcacactggtcacagtttctagcggcggtggcggaagcggaggcggtggctccggtggcggaggcagcgaaattgtgctgacacagagccccgccacactgtcactttctccaggcgaaagagccacactgagctgcaagagcagccagagcgtgctgtactccagcaaccagaagaactacctggcctggtatcagcagaagcccggccaagctcctcggctgctgatctattgggccagcacaagagagagcggcatccctgccagattttctggcagcggctctggcaccgatttcaccctgaccataagcagcctggaacctgaggacttcgccgtgtattactgccaccagtacctgagcagcctgacctttggcggaggcaccaaggtggaaatcaagcggacaacaacccctgcccccagacctcctaccccagcccctacaattgccagccagcctctgagcctgaggcccgaggcttgtagacctgctgctggcggagccgtgcacaccagaggactggatttcgcctgcgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgctgataagtttaaactgccagaacatttctctggcctaactggccggtaccggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttgataa

SEQ ID NO: 187 - DNA, 5326 bp, synthetic construct
  source 1..5326: mol_type = other DNA
atggccttaccagtgaccgccttgctcctgccgctggccttgctgctccacgccgccaggccgcaggtgcagctggttgaatctggcggcggacttgtgaagcctggcggatctctgagactgagctgtgccgccagcggcttcacctttagcacatacgccatgagctggatcagacaggcccctggcaaaggcctggaatgggtggcgtctattggcagagccggcagcacctactacagcgactctgtgaagggcagattcaccatcagccgggacaacgccaagaacagcctgtacctgcagatgaactccctgagagccgaggacaccgccgtgtactattgtgccagaggacccatctacaacgactacgacgagttcgcctattggggccagggcacactggtcacagtttctagcggcggtggcggaagcggaggcggtggctccggtggcggaggcagcgaaattgtgctgacacagagccccgccacactgtcactttctccaggcgaaagagccacactgagctgcaagagcagccagagcgtgctgtactccagcaaccagaagaactacctggcctggtatcagcagaagcccggccaagctcctcggctgctgatctattgggccagcacaagagagagcggcatccctgccagattttctggcagcggctctggcaccgatttcaccctgaccataagcagcctggaacctgaggacttcgccgtgtattactgccaccagtacctgagcagcctgacctttggcggaggcaccaaggtggaaatcaagcggacaacaacccctgcccccagacctcctaccccagcccctacaattgccagccagcctctgagcctgaggcccgaggcttgtagacctgctgctggcggagccgtgcacaccagaggactggatttcgcctgcgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgctgataagtttaaactgccagaacatttctctggcctaactggccggtaccggcttcattttttccatttactgcagaggcttcattttttccatttactgcagaggcttcattttttccatttactgcagaggcttcattttttccatttactgcagaggcttcattttttccatttactgcagaggcttcattttttccatttactgcagaactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtctggaagcggtgagggaagaggtagcctgctcacgtgcggtgatgtggaggagaaccctgggcctatgctctgtttgggctggatatttctttggcttgttgcaggagagcgaattaaaggatttaatatttcaggttgttccacaaaaaaactcctttggacatattctacaaggagtgaagaggaatttgtcttattttgtgatttaccagagccacagaaatcacatttctgccacagaaatcgactctcaccaaaacaagtccctgagcacctgcccttcatgggtagtaacgacctatctgatgtccaatggtaccaacaaccttcgaatggagatccattagaggacattaggaaaagctatcctcacatcattcaggacaaatgtacccttcactttttgaccccaggggtgaataattctgggtcatatatttgtagacccaagatgattaagagcccctatgatgtagcctgttgtgtcaagatgattttagaagttaagccccagacaaatgcatcctgtgagtattccgcatcacataagcaagacctacttcttgggagcactggctctatttcttgccccagtctcagctgccaaagtgatgcacaaagtccagcggtaacctggtacaagaatggaaaactcctctctgtggaaaggagcaaccgaatcgtagtggatgaagtttatgactatcaccagggcacatatgtatgtgattacactcagtcggatactgtgagttcgtggacagtcagagctgttgttcaagtgagaaccattgtgggagacactaaactcaaaccagatattctggatcctgtcgaggacacactggaagtagaacttggaaagcctttaactattagctgcaaagcacgatttggctttgaaagggtctttaaccctgtcataaaatggtacatcaaagattctgacctagagtgggaagtctcagtacctgaggcgaaaagtattaaatccactttaaaggatgaaatcattgagcgtaatatcatcttggaaaaagtcactcagcgtgatcttcgcaggaagtttgtttgctttgtccagaactccattggaaacacaacccagtccgtccaactgaaagaaaagagaggagtggtgctcctgtacatcctgcttggcaccatcgggaccctggtggccgtgctggcggcgagtgccctcctctacaggcactggattgaaatagtgctgctgtaccggacctaccagagcaaggatcagacgcttggggataaaaaggattttgatgctttcgtatcctatgcaaaatggagctcttttccaagtgaggccacttcatctctgagtgaagaacacttggccctgagcctatttcctgatgttttagaaaacaaatatggatatagcctgtgtttgcttgaaagagatgtggctccaggaggagtgtatgcagaagacattgtgagcattattaagagaagcagaagaggaatatttatcttgagccccaactatgtcaatggacccagtatctttgaactacaagcagcagtgaatcttgccttggatgatcaaacactgaaactcattttaattaagttctgttacttccaagagccagagtctctacctcatctcgtgaaaaaagctctcagggttttgcccacagttacttggagaggcttaaaatcagttcctcccaattctaggttctgggccaaaatgcgctaccacatgcctgtgaaaaactctcagggattcacgtggaaccagctcagaattacctctaggatttttcagtggaaaggactcagtagaacagaaaccactgggaggagctcccagcctaaggaatggtgataa

SEQ ID NO: 188 - DNA, 5245 bp, synthetic construct
  source 1..5245: mol_type = other DNA
atggccttaccagtgaccgccttgctcctgccgctggccttgctgctccacgccgccaggccgcaggtgcagctggttgaatctggcggcggacttgtgaagcctggcggatctctgagactgagctgtgccgccagcggcttcacctttagcacatacgccatgagctggatcagacaggcccctggcaaaggcctggaatgggtggcgtctattggcagagccggcagcacctactacagcgactctgtgaagggcagattcaccatcagccgggacaacgccaagaacagcctgtacctgcagatgaactccctgagagccgaggacaccgccgtgtactattgtgccagaggacccatctacaacgactacgacgagttcgcctattggggccagggcacactggtcacagtttctagcggcggtggcggaagcggaggcggtggctccggtggcggaggcagcgaaattgtgctgacacagagccccgccacactgtcactttctccaggcgaaagagccacactgagctgcaagagcagccagagcgtgctgtactccagcaaccagaagaactacctggcctggtatcagcagaagcccggccaagctcctcggctgctgatctattgggccagcacaagagagagcggcatccctgccagattttctggcagcggctctggcaccgatttcaccctgaccataagcagcctggaacctgaggacttcgccgtgtattactgccaccagtacctgagcagcctgacctttggcggaggcaccaaggtggaaatcaagcggacaacaacccctgcccccagacctcctaccccagcccctacaattgccagccagcctctgagcctgaggcccgaggcttgtagacctgctgctggcggagccgtgcacaccagaggactggatttcgcctgcgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgctgataagtttaaactgccagaacatttctctggcctaactggccggtaccggcttcattttttccatttactgcagaggcttcattttttccatttactgcagaggcttcattttttccatttactgcagaactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtctggaagcggtgagggaagaggtagcctgctcacgtgcggtgatgtggaggagaaccctgggcctatgctctgtttgggctggatatttctttggcttgttgcaggagagcgaattaaaggatttaatatttcaggttgttccacaaaaaaactcctttggacatattctacaaggagtgaagaggaatttgtcttattttgtgatttaccagagccacagaaatcacatttctgccacagaaatcgactctcaccaaaacaagtccctgagcacctgcccttcatgggtagtaacgacctatctgatgtccaatggtaccaacaaccttcgaatggagatccattagaggacattaggaaaagctatcctcacatcattcaggacaaatgtacccttcactttttgaccccaggggtgaataattctgggtcatatatttgtagacccaagatgattaagagcccctatgatgtagcctgttgtgtcaagatgattttagaagttaagccccagacaaatgcatcctgtgagtattccgcatcacataagcaagacctacttcttgggagcactggctctatttcttgccccagtctcagctgccaaagtgatgcacaaagtccagcggtaacctggtacaagaatggaaaactcctctctgtggaaaggagcaaccgaatcgtagtggatgaagtttatgactatcaccagggcacatatgtatgtgattacactcagtcggatactgtgagttcgtggacagtcagagctgttgttcaagtgagaaccattgtgggagacactaaactcaaaccagatattctggatcctgtcgaggacacactggaagtagaacttggaaagcctttaactattagctgcaaagcacgatttggctttgaaagggtctttaaccctgtcataaaatggtacatcaaagattctgacctagagtgggaagtctcagtacctgaggcgaaaagtattaaatccactttaaaggatgaaatcattgagcgtaatatcatcttggaaaaagtcactcagcgtgatcttcgcaggaagtttgtttgctttgtccagaactccattggaaacacaacccagtccgtccaactgaaagaaaagagaggagtggtgctcctgtacatcctgcttggcaccatcgggaccctggtggccgtgctggcggcgagtgccctcctctacaggcactggattgaaatagtgctgctgtaccggacctaccagagcaaggatcagacgcttggggataaaaaggattttgatgctttcgtatcctatgcaaaatggagctcttttccaagtgaggccacttcatctctgagtgaagaacacttggccctgagcctatttcctgatgttttagaaaacaaatatggatatagcctgtgtttgcttgaaagagatgtggctccaggaggagtgtatgcagaagacattgtgagcattattaagagaagcagaagaggaatatttatcttgagccccaactatgtcaatggacccagtatctttgaactacaagcagcagtgaatcttgccttggatgatcaaacactgaaactcattttaattaagttctgttacttccaagagccagagtctctacctcatctcgtgaaaaaagctctcagggttttgcccacagttacttggagaggcttaaaatcagttcctcccaattctaggttctgggccaaaatgcgctaccacatgcctgtgaaaaactctcagggattcacgtggaaccagctcagaattacctctaggatttttcagtggaaaggactcagtagaacagaaaccactgggaggagctcccagcctaaggaatggtgataa

SEQ ID NO: 189 - DNA, 5344 bp, synthetic construct
  source 1..5344: mol_type = other DNA
atggccttaccagtgaccgccttgctcctgccgctggccttgctgctccacgccgccaggccgcaggtgcagctggttgaatctggcggcggacttgtgaagcctggcggatctctgagactgagctgtgccgccagcggcttcacctttagcacatacgccatgagctggatcagacaggcccctggcaaaggcctggaatgggtggcgtctattggcagagccggcagcacctactacagcgactctgtgaagggcagattcaccatcagccgggacaacgccaagaacagcctgtacctgcagatgaactccctgagagccgaggacaccgccgtgtactattgtgccagaggacccatctacaacgactacgacgagttcgcctattggggccagggcacactggtcacagtttctagcggcggtggcggaagcggaggcggtggctccggtggcggaggcagcgaaattgtgctgacacagagccccgccacactgtcactttctccaggcgaaagagccacactgagctgcaagagcagccagagcgtgctgtactccagcaaccagaagaactacctggcctggtatcagcagaagcccggccaagctcctcggctgctgatctattgggccagcacaagagagagcggcatccctgccagattttctggcagcggctctggcaccgatttcaccctgaccataagcagcctggaacctgaggacttcgccgtgtattactgccaccagtacctgagcagcctgacctttggcggaggcaccaaggtggaaatcaagcggacaacaacccctgcccccagacctcctaccccagcccctacaattgccagccagcctctgagcctgaggcccgaggcttgtagacctgctgctggcggagccgtgcacaccagaggactggatttcgcctgcgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgctgataagtttaaactgccagaacatttctctggcctaactggccggtaccggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtctggaagcggtgagggaagaggtagcctgctcacgtgcggtgatgtggaggagaaccctgggcctatgctctgtttgggctggatatttctttggcttgttgcaggagagcgaattaaaggatttaatatttcaggttgttccacaaaaaaactcctttggacatattctacaaggagtgaagaggaatttgtcttattttgtgatttaccagagccacagaaatcacatttctgccacagaaatcgactctcaccaaaacaagtccctgagcacctgcccttcatgggtagtaacgacctatctgatgtccaatggtaccaacaaccttcgaatggagatccattagaggacattaggaaaagctatcctcacatcattcaggacaaatgtacccttcactttttgaccccaggggtgaataattctgggtcatatatttgtagacccaagatgattaagagcccctatgatgtagcctgttgtgtcaagatgattttagaagttaagccccagacaaatgcatcctgtgagtattccgcatcacataagcaagacctacttcttgggagcactggctctatttcttgccccagtctcagctgccaaagtgatgcacaaagtccagcggtaacctggtacaagaatggaaaactcctctctgtggaaaggagcaaccgaatcgtagtggatgaagtttatgactatcaccagggcacatatgtatgtgattacactcagtcggatactgtgagttcgtggacagtcagagctgttgttcaagtgagaaccattgtgggagacactaaactcaaaccagatattctggatcctgtcgaggacacactggaagtagaacttggaaagcctttaactattagctgcaaagcacgatttggctttgaaagggtctttaaccctgtcataaaatggtacatcaaagattctgacctagagtgggaagtctcagtacctgaggcgaaaagtattaaatccactttaaaggatgaaatcattgagcgtaatatcatcttggaaaaagtcactcagcgtgatcttcgcaggaagtttgtttgctttgtccagaactccattggaaacacaacccagtccgtccaactgaaagaaaagagaggagtggtgctcctgtacatcctgcttggcaccatcgggaccctggtggccgtgctggcggcgagtgccctcctctacaggcactggattgaaatagtgctgctgtaccggacctaccagagcaaggatcagacgcttggggataaaaaggattttgatgctttcgtatcctatgcaaaatggagctcttttccaagtgaggccacttcatctctgagtgaagaacacttggccctgagcctatttcctgatgttttagaaaacaaatatggatatagcctgtgtttgcttgaaagagatgtggctccaggaggagtgtatgcagaagacattgtgagcattattaagagaagcagaagaggaatatttatcttgagccccaactatgtcaatggacccagtatctttgaactacaagcagcagtgaatcttgccttggatgatcaaacactgaaactcattttaattaagttctgttacttccaagagccagagtctctacctcatctcgtgaaaaaagctctcagggttttgcccacagttacttggagaggcttaaaatcagttcctcccaattctaggttctgggccaaaatgcgctaccacatgcctgtgaaaaactctcagggattcacgtggaaccagctcagaattacctctaggatttttcagtggaaaggactcagtagaacagaaaccactgggaggagctcccagcctaaggaatggtgataa

SEQ ID NO: 190 - DNA, 5254 bp, synthetic construct
  source 1..5254: mol_type = other DNA
atggccttaccagtgaccgccttgctcctgccgctggccttgctgctccacgccgccaggccgcaggtgcagctggttgaatctggcggcggacttgtgaagcctggcggatctctgagactgagctgtgccgccagcggcttcacctttagcacatacgccatgagctggatcagacaggcccctggcaaaggcctggaatgggtggcgtctattggcagagccggcagcacctactacagcgactctgtgaagggcagattcaccatcagccgggacaacgccaagaacagcctgtacctgcagatgaactccctgagagccgaggacaccgccgtgtactattgtgccagaggacccatctacaacgactacgacgagttcgcctattggggccagggcacactggtcacagtttctagcggcggtggcggaagcggaggcggtggctccggtggcggaggcagcgaaattgtgctgacacagagccccgccacactgtcactttctccaggcgaaagagccacactgagctgcaagagcagccagagcgtgctgtactccagcaaccagaagaactacctggcctggtatcagcagaagcccggccaagctcctcggctgctgatctattgggccagcacaagagagagcggcatccctgccagattttctggcagcggctctggcaccgatttcaccctgaccataagcagcctggaacctgaggacttcgccgtgtattactgccaccagtacctgagcagcctgacctttggcggaggcaccaaggtggaaatcaagcggacaacaacccctgcccccagacctcctaccccagcccctacaattgccagccagcctctgagcctgaggcccgaggcttgtagacctgctgctggcggagccgtgcacaccagaggactggatttcgcctgcgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgctgataagtttaaactgccagaacatttctctggcctaactggccggtaccggaggaaaaactgtttcatacagaaggcgtggaggaaaaactgtttcatacagaaggcgtggaggaaaaactgtttcatacagaaggcgtactagttaggcgtgtacggtgggaggcctatataagcagagctcgtttagtgaaccgtcagatcgcctggagacgccatccacgctgttttgacctccatagaagacaccgggaccgatccagcctcgagagacccaatgctagccaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttccagcagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtctggaagcggtgagggaagaggtagcctgctcacgtgcggtgatgtggaggagaaccctgggcctatgctctgtttgggctggatatttctttggcttgttgcaggagagcgaattaaaggatttaatatttcaggttgttccacaaaaaaactcctttggacatattctacaaggagtgaagaggaatttgtcttattttgtgatttaccagagccacagaaatcacatttctgccacagaaatcgactctcaccaaaacaagtccctgagcacctgcccttcatgggtagtaacgacctatctgatgtccaatggtaccaacaaccttcgaatggagatccattagaggacattaggaaaagctatcctcacatcattcaggacaaatgtacccttcactttttgaccccaggggtgaataattctgggtcatatatttgtagacccaagatgattaagagcccctatgatgtagcctgttgtgtcaagatgattttagaagttaagccccagacaaatgcatcctgtgagtattccgcatcacataagcaagacctacttcttgggagcactggctctatttcttgccccagtctcagctgccaaagtgatgcacaaagtccagcggtaacctggtacaagaatggaaaactcctctctgtggaaaggagcaaccgaatcgtagtggatgaagtttatgactatcaccagggcacatatgtatgtgattacactcagtcggatactgtgagttcgtggacagtcagagctgttgttcaagtgagaaccattgtgggagacactaaactcaaaccagatattctggatcctgtcgaggacacactggaagtagaacttggaaagcctttaactattagctgcaaagcacgatttggctttgaaagggtctttaaccctgtcataaaatggtacatcaaagattctgacctagagtgggaagtctcagtacctgaggcgaaaagtattaaatccactttaaaggatgaaatcattgagcgtaatatcatcttggaaaaagtcactcagcgtgatcttcgcaggaagtttgtttgctttgtccagaactccattggaaacacaacccagtccgtccaactgaaagaaaagagaggagtggtgctcctgtacatcctgcttggcaccatcgggaccctggtggccgtgctggcggcgagtgccctcctctacaggcactggattgaaatagtgctgctgtaccggacctaccagagcaaggatcagacgcttggggataaaaaggattttgatgctttcgtatcctatgcaaaatggagctcttttccaagtgaggccacttcatctctgagtgaagaacacttggccctgagcctatttcctgatgttttagaaaacaaatatggatatagcctgtgtttgcttgaaagagatgtggctccaggaggagtgtatgcagaagacattgtgagcattattaagagaagcagaagaggaatatttatcttgagccccaactatgtcaatggacccagtatctttgaactacaagcagcagtgaatcttgccttggatgatcaaacactgaaactcattttaattaagttctgttacttccaagagccagagtctctacctcatctcgtgaaaaaagctctcagggttttgcccacagttacttggagaggcttaaaatcagttcctcccaattctaggttctgggccaaaatgcgctaccacatgcctgtgaaaaactctcagggattcacgtggaaccagctcagaattacctctaggatttttcagtggaaaggactcagtagaacagaaaccactgggaggagctcccagcctaaggaatggtgataa